(19) United States
(12) Patent Application Publication
(10) Pub. No.: US 2024/0124444 A1
(43) Pub. Date: Apr. 18, 2024

(54) Heterocyclic Compounds and Their Use for Parasitic Diseases

(71) Applicant: Celgene Corporation, Summit, NJ (US)

(72) Inventors: Dale E. Robinson, San Diego, CA (US); Natalie Hawryluk, San Diego, CA (US); Stacie Canan, La Jolla, CA (US)

(73) Assignee: Celgene Corporation, Summit, NJ (US)

(21) Appl. No.: 18/267,226

(22) PCT Filed: Dec. 27, 2021
(86) PCT No.: PCT/US2021/065208

§ 371 Date: Jun. 14, 2023

Related U.S. Application Data
(60) Provisional application No. 63/131,084, filed on Dec. 28, 2020.

(51) Int. Cl. C07D 471/04

(52) U.S. Cl. CPC C07D 471/04

(57) ABSTRACT

Provided herein are compounds of Formula (I) and pharmaceutically acceptable salts, tautomers, isotopologues, or stereoisomers thereof, wherein R₁, R₂, R₃, R₄ and R₅ are as defined herein (referred to herein as “Heterocyclic Compounds”), compositions comprising an effective amount of a Heterocyclic Compound, as well as methods of preventing and/or treating cryptosporidiosis and/or inhibiting a parasite or parasitic activity.

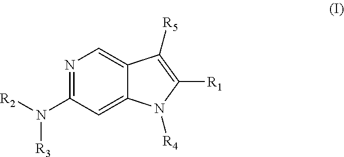

### CROSS-REFERENCE TO RELATED APPLICATIONS

This application claims benefit of priority of U.S. Provisional Application No. 63/131,084, filed Dec. 28, 2020, which is incorporated by reference herein in its entirety for any purpose.

### FIELD

Disclosed herein are Heterocyclic Compounds for preventing and/or treating cryptosporidiosis and/or inhibiting a parasite or parasitic activity. Also provided herein are Heterocyclic Compounds for use in such methods. Also disclosed herein are pharmaceutical compositions comprising Heterocyclic Compounds.

### BACKGROUND

A parasite is an organism that lives on or in a host organism and gets its food from or at the expense of its host. Cryptosporidiosis is an illness caused by tiny, one-celled parasites called Cryptosporidium. There are many species of Cryptosporidium that infect animals, some of which also infect humans. Cryptosporidium parvum and Cryptosporidium hominis are the most prevalent specifies causing disease in humans. Cryptosporidium parvum is also risk for young livestock, especially cows. Cryptosporidium is protected by an outer shell that allows it to survive outside the body for long periods of time and at varying temperature. It also makes Cryptosporidium resistant to many disinfectants.

Cryptosporidium can be spread for example by drinking or swimming in contaminated water, eating uncooked, contaminated food, and even by just touching one's mouth have having been in contact with a contaminated surface, object, person or animal. Cryptosporidium is a leading cause of waterborne disease among humans in the United States.

Symptoms of cryptosporidiosis include watery diarrhea, stomach cramps or pain, dehydration, nausea, vomiting, fever and weight loss. People with weakened immune systems may develop serious, chronic, and sometime fatal illness. Treatment of cryptosporidiosis focuses primarily on relieving one's symptoms and improving one's immune response. Nitazoxanide can be used to treat cryptosporidiosis but has to be administered multiple times daily and may take up to 5 days for symptoms such as diarrhea to resolve. Moreover, it was not shown to be superior to placebo for treating diarrhea caused by Cryptosporidium in HIV-infected or immunodeficient patients. Thus, alternative, and more effective, treatments for cryptosporidiosis and the inhibition of parasites and parasitic activity are needed.

Citation or identification of any reference in this application is not to be construed as an admission that the reference is prior art to the present application.

### SUMMARY

Provided herein are compounds of Formula I:

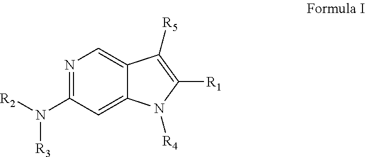

and pharmaceutically acceptable salts, tautomers, isotopologues, or stereoisomers thereof, wherein R₁, R₂, R₃, R₄, and R₅ are as defined herein (in some instances referred to herein as “Heterocyclic Compounds”).

In one aspect, provided herein are compounds as described in the instant disclosure, such as, for example, a compound of Formula I, Formula II, or Formula III, a compound from Table 1, and pharmaceutically acceptable salts, tautomers, isotopologues, or stereoisomers thereof.

In one aspect, provided herein are pharmaceutical compositions comprising an effective amount of a Heterocyclic Compound, as described herein, and a pharmaceutically acceptable carrier, excipient or vehicle. In some embodiments the pharmaceutical composition is suitable for oral, parenteral, mucosal, transdermal or topical administration.

In one aspect, provided herein are methods of treating cryptosporidiosis in a subject in need thereof. In another aspect, provided herein are uses of Heterocyclic Compounds for treating or preventing cryptosporidiosis, comprising administering to a subject affected by cryptosporidiosis an effective amount of a Heterocyclic Compound as described herein.

In one aspect, provided herein are methods of inhibiting a parasite or parasitic activity in a subject in need thereof. In another aspect, provided herein are uses of Heterocyclic Compounds for inhibiting a parasite or parasitic activity, comprising administering to a subject affected a parasite or parasitic activity an effective amount of a Heterocyclic Compound as described herein. In one aspect, the parasite is Cryptosporidium parvum. In another aspect, the parasite is Cryptosporidium hominis.

In certain embodiments, the methods described herein includes administering a therapeutically effective amount of a compound of Formula I, Formula II, Formula III, a compound from Table 1, or a pharmaceutically acceptable salt, tautomer, isotopologue, or stereoisomer thereof, to a subject.

In one aspect, provided herein is a Heterocyclic Compound for use as a medicament. In a particular embodiment, provided herein is a Heterocyclic Compound for use in a method for the treatment or prevention of cryptosporidiosis, the method comprising administering to a subject an effective amount of the Heterocyclic Compound. In a particular embodiment, provided herein is a Heterocyclic Compound for use in a method of inhibiting a parasite or parasitic activity, the method comprising administering to a subject an effective amount of the Heterocyclic Compound. In a particular embodiment, provided herein is a Heterocyclic Compound for use in a method of inhibiting Cryptosporidium parvum, the method comprising administering to a subject an effective amount of the Heterocyclic Compound. In a particular embodiment, provided herein is a Heterocyclic Compound for use in a method of inhibiting Cryptosporidium hominis, the method comprising administering to a subject an effective amount of the Heterocyclic Compound.

The present embodiments can be understood more fully by reference to the detailed description and examples, which are intended to exemplify non-limiting embodiments.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of” Consequently, the term “consisting of” can be used in place of the terms “comprising” and “including” to provide for more specific embodiments of the invention.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein and unless otherwise specified, an “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl,-n-butyl, -n-pentyl and -n-hexyl; while saturated branched alkyls include -isopropyl, -sec-butyl,-isobutyl, -tert-butyl, -isopentyl, -neopentyl, tert-pentyl, -2-methylpentyl, -3-methylpentyl, -4-methylpentyl, -2,3-dimethylbutyl and the like. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy; oxo (═O); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂.

As used herein and unless otherwise specified, a “cycloalkyl” group is a saturated, or partially saturated cyclic alkyl group of from 3 to 10 carbon atoms having a single cyclic ring or multiple condensed or bridged rings which can be optionally substituted. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

As used herein and unless otherwise specified, an “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryl groups include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

As used herein and unless otherwise specified, a “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indol-2-onyl), isoindolin-1-onyl, azaindolyl, pyrrolopyridyl (e.g., 1H-pyrrolo[2,3-b]pyridyl), indazolyl, benzimidazolyl (e.g., 1H-benzo[d]imidazolyl), azabenzimidazolyl, imidazopyridyl (e.g., 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl, 3,4-dihydroisoquinolin-1(2H)-onyl, tetrahydroquinolinyl, quinoxalinyl, and quinazolinyl groups. A heteroaryl group can be substituted or unsubstituted.

As used herein and unless otherwise specified, a “heterocyclyl” is a non-aromatic cycloalkyl in which one to four of the ring carbon atoms are independently replaced with a heteroatom. Suitable heteroatoms include oxygen, sulfur and nitrogen. In some embodiments, heterocyclyl groups include 3 to 10 ring members, whereas other such groups have 3 to 5, 3 to 6, or 4 to 9 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass partially saturated and saturated ring systems. The phrase heterocyclyl includes fused ring species, including those comprising fused aromatic and non-aromatic groups, such as, for example, 2,3-dihydrobenzo[1,4]dioxinyl, and benzo[1,3]dioxolyl. The phrase also includes bridged polycyclic ring systems containing a heteroatom such as, but not limited to, quinuclidyl.

As used herein and unless otherwise specified, a “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopropylmethyl, cyclobutylmethyl, cyclopentylmethyl, cyclohexylmethyl, cyclopropylethyl, cyclobutylethyl, cyclopentylethyl, cyclohexylethyl, cyclopentylpropyl, cyclohexylpropyl and the like.

As used herein and unless otherwise specified, an “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and aralkyl groups wherein the aryl group is fused to a cycloalkyl group such as indan-4-yl ethyl.

As used herein and unless otherwise specified, a “heterocyclylalkyl” group is a radical of the formula: -alkyl-heterocyclyl, wherein alkyl and heterocyclyl are defined above. A “heteroarylalkyl” group is a radical of the formula: -alkyl-heteroaryl, wherein alkyl and heteroaryl are defined above. A “heterocycloalkylalkyl” group is a radical of the formula: -alkyl-heterocycloalkyl, wherein alkyl and heterocycloalkyl are defined above. Substituted heterocyclylalkyl groups may be substituted at the alkyl, the heterocyclyl, or both the alkyl and the heterocyclyl portions of the group. Representative heterocylylalkyl and heteroarylalkyl groups include but are not limited to morpholin-4-yl ethyl, morpholin-4-yl propyl, furan-2-yl methyl, furan-3-yl methyl, pyridin-3-yl methyl, tetrahydrofuran-2-yl ethyl, and indol-2-yl propyl.

As used herein and unless otherwise specified, a “halogen” is fluorine, chlorine, bromine or iodine.

As used herein and unless otherwise specified, a “hydroxyalkyl” group is an alkyl group as described above substituted with one or more hydroxy groups.

As used herein and unless otherwise specified, an “alkoxy” group is —O-(alkyl), wherein alkyl is defined above. An “alkylthio” group is —S-(alkyl), wherein alkyl is defined above.

As used herein and unless otherwise specified, an “alkoxyalkyl” group is -(alkyl)-O-(alkyl), wherein alkyl is defined above.

As used herein and unless otherwise specified, a “cycloalkyloxy” group is —O-(cycloalkyl), wherein cycloalkyl is defined above.

As used herein and unless otherwise specified, an “aryloxy” group is —O-(aryl), wherein aryl is defined above.

As used herein and unless otherwise specified, a “heterocyclyloxy” group is —O-(heterocyclyl), wherein heterocyclyl is defined above. A “heteroaryloxy” group is —O-(heteroaryl), wherein heteroaryl is defined above. A “heterocycloalkyloxy” group is —O-(heterocycloalkyl), wherein heterocycloalkyl is defined above.

As used herein and unless otherwise specified, an “amino” group is a radical of the formula: —NH₂, —NH(R^{#}), or —N(R^{#})₂, wherein each R^{#} is independently an alkyl, cycloalkyl, cycloalkylalkyl, aryl, aralkyl, heterocyclyl, heteroaryl, heteroarylalkyl or heterocycloalkylalkyl group defined above, each of which is independently substituted or unsubstituted.

In one embodiment, an “amino” group is an “alkylamino” group, which is a radical of the formula: —NH-alkyl or —N(alkyl)₂, wherein each alkyl is independently defined above. The term “cycloalkylamino”, “arylamino”, “heterocyclylamino”, “heteroarylamino”, “heterocycloalkylamino”, or the like, mirrors the above description for “alkylamino” where the term “alkyl” is replaced with “cycloalkyl”, “aryl”, “heterocyclyl”, “heteroaryl”, “heterocycloalkyl”, or the like, respectively.

As used herein and unless otherwise specified, a “carboxy” group is a radical of the formula: —C(O)OH.

As used herein and unless otherwise specified, an “acyl” group is a radical of the formula: —C(O)(R^{#}), wherein R^{#} is defined above. A “formyl” group is a radical of the formula: —C(O)H.

As used herein and unless otherwise specified, an “amido” group is a radical of the formula: —C(O)—NH₂, —C(O)—NH(R^{#}), —C(O)—N(R^{#})₂, —NH—C(O)H, —NH—C(O)—(R^{#}), —N(R^{#})—C(O)H, or —N(R^{#})—C(O)—(R^{#}), wherein each R^{#} is independently defined above.

In one embodiment, an “amido” group is an “aminocarbonyl” group, which is a radical of the formula: —C(O)—NH₂, —C(O)—NH(R^{#}), —C(O)—N(R^{#})₂, wherein each R^{#} is independently defined above.

In one embodiment, an “amido” group is an “acylamino” group, which is a radical of the formula: —NH—C(O)H, —NH—C(O)—(R^{#}), —N(R^{#})—C(O)H, or —N(R^{#})—C(O)—(R^{#}), wherein each R^{#} is independently defined above.

As used herein and unless otherwise specified, a “sulfonylamino” group is a radical of the formula: —NHSO₂(R^{#}) or —N(R^{#})SO₂(R^{#}), wherein each R^{#} is defined above.

As used herein and unless otherwise specified, an “ester” group is a radical of the formula: —C(O)—O—(R^{#}) or —O—C(O)—(R^{#}), wherein R^{#} is defined above.

In one embodiment, an “ester” group is an “alkoxycarbonyl” group, which is a radical of the formula: —C(O)—O-(alkyl), wherein alkyl is defined above. The term “cycloalkyloxycarbonyl”, “aryloxycarbonyl”, “heterocyclyloxycarbonyl”, “heteroaryloxycarbonyl”, “heterocycloalkyloxycarbonyl”, or the like, mirrors the above description for “alkoxycarbonyl” where the term “alkoxy” is replaced with “cycloalkyloxy”, “aryloxy”, “heterocyclyloxy”, “heteroaryloxy”, “heterocycloalkyloxy”, or the like, respectively.

As used herein and unless otherwise specified, a “carbamate” group is a radical of the formula: —O—C(O)—NH₂, —O—C(O)—NH(R^{#}), —O—C(O)—N(R^{#})₂, —NH—C(O)—O—(R^{#}), or —N(R^{#})—C(O)—O—(R^{#}), wherein each R^{#} is independently defined above.

As used herein and unless otherwise specified, a “urea” group is a radical of the formula: —NH(CO)NH₂, —NHC(O)NH(R^{#}), —NHC(O)N(R^{#})₂, —N(R^{#})C(O)NH₂, —N(R^{#})C(O)NH(R^{#}), or —N(R^{#})C(O)N(R^{#})₂, wherein each R^{#} is independently defined above.

As used herein and unless otherwise specified, a “sulfinyl” group is a radical of the formula: —S(O)R^{#}, wherein R^{#} is defined above.

As used herein and unless otherwise specified, a “sulfonyl” group is a radical of the formula: —S(O)₂R^{#}, wherein R^{#} is defined above.

As used herein and unless otherwise specified, an “aminosulfonyl” group is a radical of the formula: —SO₂NH₂, —SO₂NH(R^{#}), or —SO₂N(R^{#})₂, wherein each R^{#} is independently defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen; alkyl, alkenyl, alkynyl, cycloalkyl, aryl, heterocyclyl, heteroaryl, heterocycloalky, cycloalkylalkyl, aralkyl, heterocyclylalkyl, heteroarylalkyl, heterocycloalkyalkyl, optionally further substituted; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy; oxo (═O); oxide (e.g., a nitrogen atom substituted with an oxide is called N-oxide); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂.

As used herein, the term “Heterocyclic Compound” includes compounds of Formula I, Formula II, Formula III, and Table 1 provided herein as well as pharmaceutically acceptable salts, tautomers, isotopologues, or stereoisomers thereof. For example, the term “Heterocyclic Compound” includes deuterated compounds of Formula I, Formula II, Formula III, and Table 1. In one embodiment, a “Heterocyclic Compound” is a compound set forth in Table 1.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The Heterocyclic Compounds can have chiral centers and can occur as racemates, individual enantiomers or diastereomers, and mixtures thereof. All such isomeric forms are included within the embodiments disclosed herein, including mixtures thereof.

The use of stereomerically pure forms of such Heterocyclic Compounds, as well as the use of mixtures of those forms, are encompassed by the embodiments disclosed herein. For example, mixtures comprising equal or unequal amounts of the enantiomers of a particular Heterocyclic Compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the Heterocyclic Compounds can include E and Z isomers, or a mixture thereof, and cis and trans isomers or a mixture thereof. In certain embodiments, the Heterocyclic Compounds are isolated as either the E or Z isomer. In other embodiments, the Heterocyclic Compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

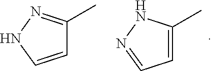

As readily understood by one skilled in the art, a wide variety of functional groups and other structures may exhibit tautomerism and all tautomers of the compounds described herein are within the scope of the present disclosure.

It should also be noted the compounds described herein can contain unnatural proportions of atomic isotopes at least one of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the sompounds described herein, whether radioactive or not, are intended to be encompassed within the scope of the embodiments provided herein. In some embodiments, there are provided isotopologues of the compounds described herein, for example, the isotopologues are carbon-13, or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It should be noted that if there is a discrepancy between a depicted structure and a name for that structure, the depicted structure is to be accorded more weight.

As used herein, “inhibit” and “inhibition” mean that a specified response of a designated activity is comparatively decreased in the presence of a Heterocyclic Compound. Inhibition of a parasite or parasitic activity, for example activity wherein the parasite is Cryptosporidium parvum, can be determined by the assays described herein.

“Treating” or “treatment” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder, disease or condition is cryptosporidiosis.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder, disease or condition is cryptosporidiosis.

The term “effective amount” in connection with a Heterocyclic Compound means an amount capable of treating or preventing a disorder, disease or condition, or symptoms thereof, disclosed herein. In one embodiment, the disorder, disease or condition is a parasitic infection.

The term “subject” or “patient” includes humans and other primates as well as domesticated and semi-domesticated animals including, but not limited to, poultry, honeybees, cows, sheep, cattle, goats, pigs, horses, dogs, cats, rabbits, rats, mice and the like. The term “poultry” encompasses all types of domestic fowl, including, but not limited to chickens, turkey, ducks, geese, the ratite group of birds and game birds. In certain embodiments, the subject is a human. In certain embodiments, the subject is a dog. In certain embodiments, the subject is a cat. In certain embodiments, the subject is a livestock. In certain embodiments, the subject is a cow. In certain embodiments, the subject is a sheep. In another embodiment, the subject is a goat.

The term “combination” or administration “in combination” includes administration as a mixture, simultaneous administration using separate formulations, and consecutive administration in any order.

The term “cryptosporidiosis” as used herein refers to a disease caused by microscopic parasites called Cryptosporidium. There are numerous species of these parasites, including, but not limited to, Cryptosporidium parvum and Cryptosporidium hominis.

Unless otherwise defined, the technical and scientific terms used herein have the same meaning as commonly understood by one of ordinary skill in the art to which this application belongs.

Surprisingly, it was found that compounds of Formula I, Formula II and Formula III and of Table 1 disclosed herein are effective in the treatment of cryptosporidiosis as well as in the inhibition of parasites or parasitic activity related to Cryptosporidium. In vitro results demonstrated that compounds of Formula I, Formula II and Formula III and of Table 1 disclosed herein are effective against C. parvum. Therefore, the compounds disclosed herein have the potential to be potent anti-parasite drugs.

### COMPOUNDS

Provided herein are compounds having the following Formula I:

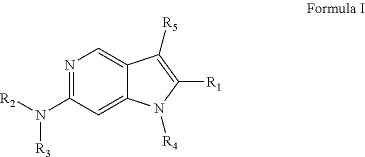

and pharmaceutically acceptable salts, tautomers, isotopologues, and stereoisomers thereof, wherein:
R₁ is chosen from H, substituted or unsubstituted C₁-C₄ alkyl, substituted or unsubstituted 3-6 membered cycloalkyl, substituted or unsubstituted 6-membered heterocyclyl, substituted or unsubstituted 6-membered aryl, substituted or unsubstituted 5-6 membered heteroaryl, and —CN;
R₂ is chosen from H and —CH₃;
R₃ is chosen from H, substituted or unsubstituted C₁-C₄ alkyl, substituted or unsubstituted 4-6 membered cycloalkyl, substituted or unsubstituted 4-9 membered heterocyclyl, substituted or unsubstituted 6-membered aryl, substituted or unsubstituted 5-membered heteroaryl, —CH₂-cyclopropyl, —C(═O)—R₆, —C(═O)CH₂-R₇, —C(═O)—O—R₈, —C(═O)NR₉-R₁₀, —C(═N)R₁₁, and —S(═O)₂-R₁₂;
R₄ is chosen from H and substituted or unsubstituted C₁-C₄ alkyl;
R₅ group chosen from H and substituted or unsubstituted C₁-C₄ alkyl;
R₆ is chosen from substituted or unsubstituted C₁-C₄ alkyl, substituted or unsubstituted 3-7 membered cycloalkyl, substituted or unsubstituted 5-6 membered heterocyclyl, substituted or unsubstituted 6 membered aryl, and substituted or unsubstituted 5-9 membered heteroaryl;
R₇ is chosen from substituted or unsubstituted 5 membered cycloalkyl, substituted or unsubstituted 4-6 membered heterocyclyl, and —N(CH₃)₂;
R₈ is chosen from substituted or unsubstituted C₁ alkyl, substituted or unsubstituted 5-6 membered cycloalkyl, and substituted or unsubstituted 5 membered heterocyclyl;
R₉ and R₁₀ are independently chosen from H, substituted or unsubstituted C₁ alkyl, substituted or unsubstituted 5-6 membered cycloalkyl, or R₉ and R₁₀ are taken together with the N to which they are attached and form a substituted or unsubstituted 5 membered heterocyclyl;
R₁₁ is a substituted or unsubstituted 3 membered cycloalkyl; and
R₁₂ is a substituted or unsubstituted 3 membered cycloalkyl; and
wherein when R₁ is not a substituted or unsubstituted 5-6 membered heteroaryl, then R₃ is chosen from substituted or unsubstituted 4-9 membered heterocyclyl, substituted or unsubstituted 6-membered aryl, substituted or unsubstituted 5-membered heteroaryl, —C(═O)—R₆, —C(═O)—O—R₈, —C(═O)NR₉-R₁₀,—S(═O)₂-R₁₂.

In some embodiments, R₁ is chosen from substituted or unsubstituted C₁-C₄ cycloalkyl, substituted or unsubstituted 6 membered aryl, substituted or unsubstituted 5-6 membered heteroaryl, —CF₃, and —CN.

In certain embodiments, R₁ is chosen from a substituted or unsubstituted 4-6 membered cycloalkyl, substituted or unsubstituted 6 membered aryl, and substituted or unsubstituted 5 membered heteroaryl.

In some embodiments, R₃ is chosen from substituted or unsubstituted C₁-C₄ alkyl, substituted or unsubstituted 4-6 membered cycloalkyl, substituted or unsubstituted 4-9 membered heterocyclyl, substituted or unsubstituted 6 membered aryl, substituted or unsubstituted 5 membered heteroaryl, —CH₂-cyclopropyl, and —C(═O)—R₆; and R₆ is chosen from substituted or unsubstituted 3-7 membered cycloalkyl, and substituted or unsubstituted 5-9 membered heteroaryl.

In certain embodiments, R₃ is chosen from substituted or unsubstituted C₁-C₄ alkyl, substituted or unsubstituted 4 membered cycloalkyl, substituted or unsubstituted 5-6 membered heterocyclyl, substituted or unsubstituted 6 membered aryl, substituted or unsubstituted 5 membered heteroaryl, and —CH₂-cyclopropyl.

In some embodiments, R₃ is =C(═O)—R₆; and wherein R₆ is chosen from substituted or unsubstituted C₁-C₄ alkyl, substituted or unsubstituted 3-7 membered cycloalkyl, substituted or unsubstituted 5-6 membered heterocyclyl, substituted or unsubstituted 6 membered aryl, and substituted or unsubstituted 5-9 membered heteroaryl.

In certain embodiments, R₂ is H.

In one embodiment, the compound is a compound of Formula II.

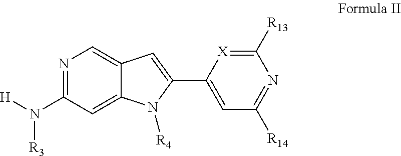

and pharmaceutically acceptable salts, tautomers, isotopologues, and stereoisomers thereof, wherein:
X is chosen from N or CH;
R₃ is chosen from substituted or unsubstituted C₁-C₄ alkyl, substituted or unsubstituted 4 membered cycloalkyl, substituted or unsubstituted 5-6 membered heterocyclyl, substituted or unsubstituted 6 membered aryl, substituted or unsubstituted 5 membered heteroaryl, —CH₂-cyclopropyl, and —C(═O)—RC;
R₄ is chosen from H and —CH₃;
R₆ is chosen from substituted or unsubstituted 3 membered cycloalkyl and substituted or unsubstituted 5 membered heteroaryl;
R₁₃ and R₁₄ are each independently chosen from H, substituted or unsubstituted C₁-C₄ alkyl, substituted or unsubstituted C₁-C₂ alkoxyl, substituted or unsubstituted amino, substituted or unsubstituted 5 membered heterocyclyl, and —CN.

In some embodiments, R₃ is chosen from substituted or unsubstituted 6 membered heterocyclyl and —C(═O)—R₆.

In certain embodiments, R₁₃ and R₁₄ are independently chosen from H, substituted or unsubstituted C₁ alkyl, and substituted or unsubstituted amino.

In other embodiments, the compound is a compound of Formula III:

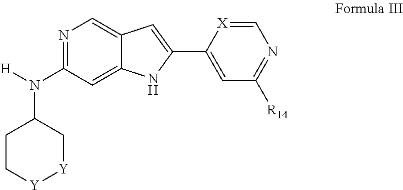

and pharmaceutically acceptable salts, tautomers, isotopologues, and stereoisomers thereof, wherein:
X is chosen from N or CH;
R₁₄ is chosen from H, substituted or unsubstituted C₁-C₂ alkyl, substituted or unsubstituted C₁-C₂ alkoxyl, and substituted or unsubstituted amino; and each Y is independently chosen from CH₂ and O.

In some embodiments, R₁₄ is chosen from substituted or unsubstituted C₁ alkyl and substituted or unsubstituted amino.

In certain embodiments, R₁₄ is —NH(CH₂)—CF₃.

In some embodiments, the compound is chosen from N-(tetrahydro-2H-pyran-4-yl)-2-(2-(2,2,2-trifluoroethylamino)pyrimidin-4-yl)-1H-pyrrolo[3,2-c]pyridin-6-amine;
N-(3-methyl-5-(1H-1,2,4-triazol-1-yl)phenyl)-2-(2-(2,2,2-trifluoroethylamino)pyrimidin-4-yl)-1H-pyrrolo[3,2-c]pyridin-6-amine;
N-(3-methyl-2-(2-methylpyridin-4-yl)-1H-pyrrolo[3,2-c]pyridin-6-yl)cyclopropanecarboxamide;
1-methyl-N-(2-(2-(2,2,2-trifluoroethylamino)pyrimidin-4-yl)-1H-pyrrolo[3,2-c]pyridin-6-yl)-1H-pyrazole-4-carboxamide;
N-(2-(2-methylpyridin-4-yl)-1H-pyrrolo[3,2-c]pyridin-6-yl)bicyclo[3.1.0]hexane-6-carboxamide;
6-(phenylamino)-1H-pyrrolo[3,2-c]pyridine-2-carbonitrile;
1,3-dimethyl-N-(2-(2-(2,2,2-trifluoroethylamino)pyrimidin-4-yl)-1H-pyrrolo[3,2-c]pyridin-6-yl)-1H-pyrazole-4-carboxamide;
N-(2-(2-(cyclopropylamino)pyridin-4-yl)-1H-pyrrolo[3,2-c]pyridin-6-yl)cyclopropanecarboxamide;
N-(2-(2-methylpyridin-4-yl)-1H-pyrrolo[3,2-c]pyridin-6-yl)cyclopropanecarboxamide;
(1s,3s)-N-(2-(2-ethoxypyrimidin-4-yl)-1H-pyrrolo[3,2-c]pyridin-6-yl)-3-hydroxycyclobutanecarboxamide;
N-(2-(trifluoromethyl)-1H-pyrrolo[3,2-c]pyridin-6-yl)cyclopropanecarboxamide;
2-(2-methylpyridin-4-yl)-N-(tetrahydro-2H-pyran-4-yl)-1H-pyrrolo[3,2-c]pyridin-6-amine;
(R)-1-methyl-N-(2-(2-(1,1,1-trifluoropropan-2-ylamino)pyrimidin-4-yl)-1H-pyrrolo[3,2-c]pyridin-6-yl)-1H-pyrazole-4-carboxamide;
N-(2-(2-(2,2,2-trifluoroethoxy)pyrimidin-4-yl)-1H-pyrrolo[3,2-c]pyridin-6-yl)cyclopropanecarboxamide;
6-(3-morpholinophenylamino)-1H-pyrrolo[3,2-c]pyridine-2-carbonitrile;
6-(3-(tetrahydro-2H-pyran-4-yl)phenylamino)-1H-pyrrolo[3,2-c]pyridine-2-carbonitrile;
N-(2-(2-methoxypyrimidin-4-yl)-1-methyl-1H-pyrrolo[3,2-c]pyridin-6-yl)cyclopropanecarboxamide;
N-(2-(2-(2-methoxyethylamino)pyridin-4-yl)-1H-pyrrolo[3,2-c]pyridin-6-yl)cyclopropanecarboxamide;
2-methyl-N-(2-(2-methylpyridin-4-yl)-1H-pyrrolo[3,2-c]pyridin-6-yl)cyclopropanecarboxamide;
N-(3-methoxyphenyl)-2-(1H-pyrazol-4-yl)-1H-pyrrolo[3,2-c]pyridin-6-amine;
N-(2-(2-methoxypyrimidin-4-yl)-1H-pyrrolo[3,2-c]pyridin-6-yl)cyclopropanecarboxamide;
(1r,3r)-3-hydroxy-N-(2-(2-methoxypyrimidin-4-yl)-1-methyl-1H-pyrrolo[3,2-c]pyridin-6-yl)cyclobutanecarboxamide;
2-methyl-5-(2-(pyridin-4-yl)-1H-pyrrolo[3,2-c]pyridin-6-ylamino)isoindolin-1-one;
2-(oxazol-5-yl)-N-(3-(tetrahydro-2H-pyran-4-yl)phenyl)-1H-pyrrolo[3,2-c]pyridin-6-amine;
N-(2-(oxazol-5-yl)-1H-pyrrolo[3,2-c]pyridin-6-yl)cyclopropanecarboxamide;
N-(2-(2,6-difluoropyridin-4-yl)-1H-pyrrolo[3,2-c]pyridin-6-yl)cyclopropanecarboxamide;
2-(oxazol-5-yl)-N-(tetrahydro-2H-pyran-4-yl)-1H-pyrrolo[3,2-c]pyridin-6-amine;
N-(1-methyl-2-(1H-pyrazol-4-yl)-1H-pyrrolo[3,2-c]pyridin-6-yl)cyclopropanecarboxamide;
N-(2-(2-methyloxazol-5-yl)-1H-pyrrolo[3,2-c]pyridin-6-yl)cyclopropanecarboxamide;
N-(2-(2-cyanopyridin-4-yl)-1H-pyrrolo[3,2-c]pyridin-6-yl)cyclopropanecarboxamide;
(R)-2-(2-methylpyridin-4-yl)-N-(tetrahydrofuran-3-yl)-1H-pyrrolo[3,2-c]pyridin-6-amine;
2-(pyridin-4-yl)-N-(3-(tetrahydro-2H-pyran-4-yl)phenyl)-1H-pyrrolo[3,2-c]pyridin-6-amine;
2-(2-methylpyridin-4-yl)-N-(4-methyltetrahydro-2H-pyran-4-yl)-1H-pyrrolo[3,2-c]pyridin-6-amine;
N-(2-(2-ethoxypyrimidin-4-yl)-1H-pyrrolo[3,2-c]pyridin-6-yl)cyclopropanecarboxamide;
N-(2-(2-methylpyridin-4-yl)-1H-pyrrolo[3,2-c]pyridin-6-yl)bicyclo[4.1.0]heptane-7-carboxamide;
N-(2-(2-(trifluoromethyl)pyrimidin-4-yl)-1H-pyrrolo[3,2-c]pyridin-6-yl)cyclopropanecarboxamide;
N-(2-(pyridin-3-yl)-1H-pyrrolo[3,2-c]pyridin-6-yl)cyclopropanecarboxamide;
2-(6-methylpyrimidin-4-yl)-N-(tetrahydro-2H-pyran-4-yl)-1H-pyrrolo[3,2-c]pyridin-6-amine;
1-methyl-2-(6-methylpyrimidin-4-yl)-N-(tetrahydro-2H-pyran-4-yl)-1H-pyrrolo[3,2-c]pyridin-6-amine;
2-(2-methoxypyrimidin-4-yl)-N-(1-(tetrahydro-2H-pyran-4-yl)-1H-pyrazol-4-yl)-1H-pyrrolo[3,2-c]pyridin-6-amine;
N-(2-m-tolyl-1H-pyrrolo[3,2-c]pyridin-6-yl)cyclopropanecarboxamide;
N-(2-(5-methyl-1H-pyrazol-4-yl)-1H-pyrrolo[3,2-c]pyridin-6-yl)cyclopropanecarboxamide; (S)-tetrahydrofuran-3-yl 2-(2-methylpyridin-4-yl)-1H-pyrrolo[3,2-c]pyridin-6-ylcarbamate;
1-methyl-N-(1-methyl-2-(2-(2,2,2-trifluoroethylamino)pyrimidin-4-yl)-1H-pyrrolo[3,2-c]pyridin-6-yl)-1H-pyrazole-4-carboxamide;
(1R,2R)—N-(2-(2-methoxypyrimidin-4-yl)-1H-pyrrolo[3,2-c]pyridin-6-yl)-2-methylcyclopropanecarboxamide;
N-(1-methyl-2-(6-methylpyrimidin-4-yl)-1H-pyrrolo[3,2-c]pyridin-6-yl)cyclopropanecarboxamide;
N-(2-(thiazol-5-yl)-1H-pyrrolo[3,2-c]pyridin-6-yl)cyclopropanecarboxamide;
N-(2-(6-methylpyrimidin-4-yl)-1H-pyrrolo[3,2-c]pyridin-6-yl)cyclopropanecarboxamide;
2-(2-methylpyrimidin-4-yl)-N-(tetrahydro-2H-pyran-4-yl)-1H-pyrrolo[3,2-c]pyridin-6-amine;
N-(2-(2-methylpyrimidin-4-yl)-1H-pyrrolo[3,2-c]pyridin-6-yl)cyclopropanecarboxamide;
N-(1-methyl-2-(1H-1,2,4-triazol-3-yl)-1H-pyrrolo[3,2-c]pyridin-6-yl)cyclopropanecarboxamide; (S)-2-(2-methylpyridin-4-yl)-N-(tetrahydrofuran-3-yl)-1H-pyrrolo[3,2-c]pyridin-6-amine;
N-(2-(3,3-difluorocyclobutyl)-1H-pyrrolo[3,2-c]pyridin-6-yl)cyclopropanecarboxamide;
4-(6-(3-(tetrahydro-2H-pyran-4-yl)phenylamino)-1H-pyrrolo[3,2-c]pyridin-2-yl)picolinonitrile;
N-(1-(2-hydroxyethyl)-2-(6-methylpyrimidin-4-yl)-1H-pyrrolo[3,2-c]pyridin-6-yl)cyclopropanecarboxamide;
4-(6-(1-methyl-1H-pyrazol-4-ylamino)-1H-pyrrolo[3,2-c]pyridin-2-yl)picolinonitrile;
N-(1-methyl-2-(1-methyl-1H-1,2,4-triazol-3-yl)-1H-pyrrolo[3,2-c]pyridin-6-yl)cyclopropanecarboxamide;
N-(2-(2-ethoxypyrimidin-4-yl)-1H-pyrrolo[3,2-c]pyridin-6-yl)bicyclo[3.1.0]hexane-6-carboxamide;
1-methyl-2-(2-methylpyridin-4-yl)-N-(tetrahydro-2H-pyran-4-yl)-1H-pyrrolo[3,2-c]pyridin-6-amine;
N-(2-(1-methyl-1H-imidazol-4-yl)-1H-pyrrolo[3,2-c]pyridin-6-yl)cyclopropanecarboxamide;
N-(2-(2-cyclopropyloxazol-5-yl)-1H-pyrrolo[3,2-c]pyridin-6-yl)cyclopropanecarboxamide;
2-(2-methoxypyrimidin-4-yl)-N-(tetrahydro-2H-pyran-4-yl)-1H-pyrrolo[3,2-c]pyridin-6-amine;
(1r,3r)-3-hydroxy-N-(2-(2-methoxypyrimidin-4-yl)-1H-pyrrolo[3,2-c]pyridin-6-yl)cyclobutanecarboxamide;
1-methyl-N-(2-(oxazol-5-yl)-1H-pyrrolo[3,2-c]pyridin-6-yl)-1H-pyrazole-4-carboxamide;
N-(2-(1-methyl-1H-1,2,4-triazol-3-yl)-1H-pyrrolo[3,2-c]pyridin-6-yl)cyclopropanecarboxamide;
N-(3-(1H-1,2,4-triazol-3-yl)phenyl)-2-(2-methylpyridin-4-yl)-1H-pyrrolo[3,2-c]pyridin-6-amine;
N-(cyclopropylmethyl)-2-(2-methylpyridin-4-yl)-1H-pyrrolo[3,2-c]pyridin-6-amine;
N-(2-(pyridin-4-yl)-1H-pyrrolo[3,2-c]pyridin-6-yl)tetrahydro-2H-pyran-4-carboxamide;
N-(1-methyl-2-(6-methylpyridin-2-yl)-1H-pyrrolo[3,2-c]pyridin-6-yl)cyclopropanecarboxamide;
1-methyl-2-(1-methyl-1H-pyrazol-4-yl)-N-phenyl-1H-pyrrolo[3,2-c]pyridin-6-amine;
N-(tetrahydro-2H-pyran-4-yl)-2-(trifluoromethyl)-1H-pyrrolo[3,2-c]pyridin-6-amine; 2-(pyridin-3-yl)-N-(tetrahydro-2H-pyran-4-yl)-1H-pyrrolo[3,2-c]pyridin-6-amine;
N-(1-methyl-2-(4-methylpyridin-2-yl)-1H-pyrrolo[3,2-c]pyridin-6-yl)cyclopropanecarboxamide;
N-(1-methyl-2-(oxazol-5-yl)-1H-pyrrolo[3,2-c]pyridin-6-yl)cyclopropanecarboxamide;
N-(1-(2-aminoethyl)-2-(6-methylpyrimidin-4-yl)-1H-pyrrolo[3,2-c]pyridin-6-yl)cyclopropanecarboxamide;
N-(2-(trifluoromethyl)-1H-pyrrolo[3,2-c]pyridin-6-yl)cyclobutanecarboxamide;
N-(1-methyl-2-(trifluoromethyl)-1H-pyrrolo[3,2-c]pyridin-6-yl)cyclopropanecarboxamide;
N-(2-(pyridin-4-yl)-1H-pyrrolo[3,2-c]pyridin-6-yl)isobutyramide;
4-(6-(3-(4H-1,2,4-triazol-3-yl)phenylamino)-1H-pyrrolo[3,2-c]pyridin-2-yl)picolinonitrile;
N-(1-(2-hydroxyethyl)-2-(2-(2,2,2-trifluoroethylamino)pyrimidin-4-yl)-1H-pyrrolo[3,2-c]pyridin-6-yl)-1-methyl-1H-pyrazole-4-carboxamide;
2-(2-methylpyridin-4-yl)-1H-pyrrolo[3,2-c]pyridin-6-amine;
N-(2-(1H-imidazol-5-yl)-1H-pyrrolo[3,2-c]pyridin-6-yl)-1-methyl-1H-pyrazole-4-carboxamide;
N-(1-methyl-2-(2-methylpyridin-4-yl)-1H-pyrrolo[3,2-c]pyridin-6-yl)cyclopropanecarboxamide;
1-cyclopentyl-N-(2-(2-methylpyridin-4-yl)-1H-pyrrolo[3,2-c]pyridin-6-yl)-1H-pyrazole-4-carboxamide;
N-(2-(2-((1r,4r)-4-hydroxycyclohexyloxy)pyrimidin-4-yl)-1H-pyrrolo[3,2-c]pyridin-6-yl)-1-methyl-1H-pyrazole-4-carboxamide;
N-methyl-N-(2-(2-methylpyridin-4-yl)-1H-pyrrolo[3,2-c]pyridin-6-yl)cyclopropanecarboxamide;
N-(2-(2-methoxypyrimidin-4-yl)-1-methyl-1H-pyrrolo[3,2-c]pyridin-6-yl)-2-(oxetan-3-yl)acetamide;
N-(2-cyclopropyl-1H-pyrrolo[3,2-c]pyridin-6-yl)-1-methyl-1H-pyrazole-4-carboxamide;
N-(2-(3-hydroxypropyl)-1H-pyrrolo[3,2-c]pyridin-6-yl)-1-methyl-1H-pyrazole-4-carboxamide;
1-methyl-N-(2-(1-methyl-1H-imidazol-5-yl)-1H-pyrrolo[3,2-c]pyridin-6-yl)-1H-pyrazole-4-carboxamide;
2-(oxetan-3-yl)-N-(2-(2-(trifluoromethyl)pyrimidin-4-yl)-1H-pyrrolo[3,2-c]pyridin-6-yl)acetamide;
(1r,3r)-3-hydroxy-N-(2-(2-(trifluoromethyl)pyrimidin-4-yl)-1H-pyrrolo[3,2-c]pyridin-6-yl)cyclobutanecarboxamide;
N-(1-(2-hydroxyethyl)-2-(2-methylpyridin-4-yl)-1H-pyrrolo[3,2-c]pyridin-6-yl)cyclopropanecarboxamide;
N-(2-(tetrahydro-2H-pyran-4-yl)-1H-pyrrolo[3,2-c]pyridin-6-yl)cyclopropanecarboxamide;
1-methyl-N-(2-(2-methylpyridin-4-yl)-1H-pyrrolo[3,2-c]pyridin-6-yl)cyclopropanecarboxamide;
N-(2-(2-methylpyridin-4-yl)-1H-pyrrolo[3,2-c]pyridin-6-yl)cyclopropanecarboximidamide;
N-(2-(2-methylpyridin-4-yl)-1H-pyrrolo[3,2-c]pyridin-6-yl)cyclopropanesulfonamide;
N-(2-(6-methylpyrimidin-4-yl)-1H-pyrrolo[3,2-c]pyridin-6-yl)cyclobutanecarboxamide;
N-(2-(2-methylpyrimidin-4-yl)-1H-pyrrolo[3,2-c]pyridin-6-yl)cyclobutanecarboxamide;
N-(1-methyl-2-(2-methylpyrimidin-4-yl)-1H-pyrrolo[3,2-c]pyridin-6-yl)cyclopropanecarboxamide;
N-(2-(2-methylthiazol-5-yl)-1H-pyrrolo[3,2-c]pyridin-6-yl)cyclopropanecarboxamide;
N-(1-methyl-2-(1-methyl-1H-pyrazol-4-yl)-1H-pyrrolo[3,2-c]pyridin-6-yl)cyclopropanecarboxamide;
N-(2-(2-(trifluoromethyl)thiazol-5-yl)-1H-pyrrolo[3,2-c]pyridin-6-yl)cyclopropanecarboxamide;
N-(2-(4-(trifluoromethyl)thiazol-2-yl)-1H-pyrrolo[3,2-c]pyridin-6-yl)cyclopropanecarboxamide;
N-(2-(trifluoromethyl)-1H-pyrrolo[3,2-c]pyridin-6-yl)tetrahydro-2H-pyran-4-carboxamide;
N-(2-(trifluoromethyl)-1H-pyrrolo[3,2-c]pyridin-6-yl)cyclopropanesulfonamide; methyl 2-(trifluoromethyl)-1H-pyrrolo[3,2-c]pyridin-6-ylcarbamate;
1,1-dimethyl-3-(2-(trifluoromethyl)-1H-pyrrolo[3,2-c]pyridin-6-yl)urea;
1-methyl-N-(2-(trifluoromethyl)-1H-pyrrolo[3,2-c]pyridin-6-yl)piperidine-4-carboxamide;
N-(2-(2-(trifluoromethyl)oxazol-5-yl)-1H-pyrrolo[3,2-c]pyridin-6-yl)cyclopropanecarboxamide;
N-(2-(trifluoromethyl)-1H-pyrrolo[3,2-c]pyridin-6-yl)cyclopentanecarboxamide;
N-(1-methylpiperidin-4-yl)-2-(2-methylpyridin-4-yl)-1H-pyrrolo[3,2-c]pyridin-6-amine;
2-(2-methylpyridin-4-yl)-N-(oxetan-3-yl)-1H-pyrrolo[3,2-c]pyridin-6-amine;
N-methyl-2-(2-methylpyridin-4-yl)-N-(tetrahydro-2H-pyran-4-yl)-1H-pyrrolo[3,2-c]pyridin-6-amine;
N-(3-methyl-1H-pyrrolo[3,2-c]pyridin-6-yl)cyclopropanecarboxamide;
N-(1-(2-hydroxyethyl)-2-(trifluoromethyl)-1H-pyrrolo[3,2-c]pyridin-6-yl)cyclopropanecarboxamide;
1-methyl-N-(1-methylpiperidin-4-yl)-2-(6-methylpyrimidin-4-yl)-1H-pyrrolo[3,2-c]pyridin-6-amine;
N-(1-methyl-2-(6-methylpyrimidin-4-yl)-1H-pyrrolo[3,2-c]pyridin-6-yl)isobutyramide;
N-(1-methyl-2-(6-methylpyrimidin-4-yl)-1H-pyrrolo[3,2-c]pyridin-6-yl)acetamide;
1-methyl-2-(6-methylpyrimidin-4-yl)-N-(piperidin-4-yl)-1H-pyrrolo[3,2-c]pyridin-6-amine;
2-cyclobutyl-N-(tetrahydro-2H-pyran-4-yl)-1H-pyrrolo[3,2-c]pyridin-6-amine;
N-(1-ethyl-2-(6-methylpyrimidin-4-yl)-1H-pyrrolo[3,2-c]pyridin-6-yl)cyclopropanecarboxamide;
2-(1-methyl-1H-imidazol-4-yl)-N-(tetrahydro-2H-pyran-4-yl)-1H-pyrrolo[3,2-c]pyridin-6-amine;
N-(1-(2-(dimethylamino)ethyl)-2-(6-methylpyrimidin-4-yl)-1H-pyrrolo[3,2-c]pyridin-6-yl)cyclopropanecarboxamide;
1-methyl-2-(6-methylpyrimidin-4-yl)-1H-pyrrolo[3,2-c]pyridin-6-amine; N,1-dimethyl-2-(6-methylpyrimidin-4-yl)-1H-pyrrolo[3,2-c]pyridin-6-amine;
2-(dimethylamino)-N-(1-methyl-2-(6-methylpyrimidin-4-yl)-1H-pyrrolo[3,2-c]pyridin-6-yl)acetamide;
N-(1-methyl-2-(6-methylpyrimidin-4-yl)-1H-pyrrolo[3,2-c]pyridin-6-yl)-2-(pyrrolidin-1-yl)acetamide;
N-(1-methyl-2-(pyridin-2-yl)-1H-pyrrolo[3,2-c]pyridin-6-yl)cyclopropanecarboxamide;
N-(1-methyl-2-(6-(trifluoromethyl)pyrimidin-4-yl)-1H-pyrrolo[3,2-c]pyridin-6-yl)cyclopropanecarboxamide;
N-(2-(2-(cyclopropylmethylamino)pyrimidin-4-yl)-1-methyl-1H-pyrrolo[3,2-c]pyridin-6-yl)-1-isopropyl-1H-pyrazole-4-carboxamide;
N-(2-(2-(cyclopropylamino)pyrimidin-4-yl)-1-methyl-1H-pyrrolo[3,2-c]pyridin-6-yl)-1-isopropyl-1H-pyrazole-4-carboxamide;
1-methyl-N-(1-methyl-2-(2-(3,3,3-trifluoropropylamino)pyrimidin-4-yl)-1H-pyrrolo[3,2-c]pyridin-6-yl)-1H-pyrazole-4-carboxamide;
3-methyl-N-(2-(pyridin-4-yl)-1H-pyrrolo[3,2-c]pyridin-6-yl)butanamide;
(R)—N-(2-(pyridin-4-yl)-1H-pyrrolo[3,2-c]pyridin-6-yl)tetrahydrofuran-3-carboxamide; (S)-1,3-dimethyl-N-(2-(2-(1,1,1-trifluoropropan-2-yloxy)pyrimidin-4-yl)-1H-pyrrolo[3,2-c]pyridin-6-yl)-1H-pyrazole-4-carboxamide;
N-(2-(pyridin-4-yl)-1H-pyrrolo[3,2-c]pyridin-6-yl)cyclopentanecarboxamide;
1-methyl-2-(pyridin-4-yl)-N-(1-(tetrahydro-2H-pyran-4-yl)-1H-pyrazol-4-yl)-1H-pyrrolo[3,2-c]pyridin-6-amine;
N-(2-(2-ethoxypyrimidin-4-yl)-1H-pyrrolo[3,2-c]pyridin-6-yl)-2-(oxetan-3-yl)acetamide;
N-(3-methoxy-5-(1H-1,2,4-triazol-1-yl)phenyl)-2-(pyridin-4-yl)-1H-pyrrolo[3,2-c]pyridin-6-amine;
N-(2-(2-(1,1-difluoropropan-2-yloxy)pyrimidin-4-yl)-1H-pyrrolo[3,2-c]pyridin-6-yl)-1-methyl-1H-pyrazole-4-carboxamide;
(R)—N-(1-methyl-2-(2-(2,2,2-trifluoroethylamino)pyrimidin-4-yl)-1H-pyrrolo[3,2-c]pyridin-6-yl)-1-(tetrahydrofuran-3-yl)-1H-pyrazole-4-carboxamide;
N-(2-(pyridin-4-yl)-1H-pyrrolo[3,2-c]pyridin-6-yl)-2-(tetrahydro-2H-pyran-4-yl)acetamide;
N-(2-(2-(2,2-difluoroethylamino)pyrimidin-4-yl)-1-methyl-1H-pyrrolo[3,2-c]pyridin-6-yl)-1-methyl-1H-pyrazole-4-carboxamide;
6-(3-(1H-1,2,4-triazol-3-yl)phenylamino)-1H-pyrrolo[3,2-c]pyridine-2-carbonitrile;
N-(2-(2-(2-fluoroethoxy)pyrimidin-4-yl)-1H-pyrrolo[3,2-c]pyridin-6-yl)-1-isopropyl-1H-pyrazole-4-carboxamide;
2-(oxazol-5-yl)-N-(1-(tetrahydro-2H-pyran-4-yl)-1H-pyrazol-4-yl)-1H-pyrrolo[3,2-c]pyridin-6-amine;
6-(3-methoxy-5-(tetrahydro-2H-pyran-4-yl)phenylamino)-1H-pyrrolo[3,2-c]pyridine-2-carbonitrile;
1-isopropyl-N-(2-(2-(1,1,1-trifluoropropan-2-yloxy)pyrimidin-4-yl)-1H-pyrrolo[3,2-c]pyridin-6-yl)-1H-pyrazole-4-carboxamide;
N-(3-(1H-1,2,4-triazol-3-yl)phenyl)-2-(2-amino-6-methylpyridin-4-yl)-1H-pyrrolo[3,2-c]pyridin-6-amine;
6-(1-(tetrahydro-2H-pyran-4-yl)-1H-pyrazol-4-ylamino)-1H-pyrrolo[3,2-c]pyridine-2-carbonitrile;
(R)-tetrahydrofuran-3-yl 2-(2-methylpyridin-4-yl)-1H-pyrrolo[3,2-c]pyridin-6-ylcarbamate;
(1R,2R)—N-(2-(2-ethoxypyrimidin-4-yl)-1H-pyrrolo[3,2-c]pyridin-6-yl)-2-methylcyclopropanecarboxamide;
N-(2-(pyridin-4-yl)-1H-pyrrolo[3,2-c]pyridin-6-yl)-1-(tetrahydro-2H-pyran-4-yl)-1H-pyrazole-4-carboxamide;
N-(2-(2-methylpyridin-4-yl)-1H-pyrrolo[3,2-c]pyridin-6-yl)tetrahydro-2H-pyran-3-carboxamide;
(S)—N-(2-(2-methoxypyrimidin-4-yl)-1H-pyrrolo[3,2-c]pyridin-6-yl)-1-((tetrahydrofuran-3-yl)methyl)-1H-pyrazole-4-carboxamide;
(S)—N-(2-(pyridin-4-yl)-1H-pyrrolo[3,2-c]pyridin-6-yl)tetrahydrofuran-3-carboxamide;
N-(2-(2-ethoxypyrimidin-4-yl)-1H-pyrrolo[3,2-c]pyridin-6-yl)bicyclo[4.1.0]heptane-7-carboxamide;
N-(2-(2-(cyclopropylmethylamino)pyrimidin-4-yl)-1-methyl-1H-pyrrolo[3,2-c]pyridin-6-yl)-1-methyl-1H-pyrazole-4-carboxamide;
N-(2-(2-methylpyridin-4-yl)-1H-pyrrolo[3,2-c]pyridin-6-yl)isobutyramide;
(1r,3r)-3-methoxy-N-(2-(2-methylpyridin-4-yl)-1H-pyrrolo[3,2-c]pyridin-6-yl)cyclobutanecarboxamide;
1-isopropyl-N-(2-(pyridin-4-yl)-1H-pyrrolo[3,2-c]pyridin-6-yl)-1H-pyrazole-4-carboxamide;
(1s,3s)-3-hydroxy-N-(2-(2-methoxypyrimidin-4-yl)-1-methyl-1H-pyrrolo[3,2-c]pyridin-6-yl)cyclobutanecarboxamide;
6-(1-methyl-1H-pyrazol-4-ylamino)-1H-pyrrolo[3,2-c]pyridine-2-carbonitrile;
N-(2-(2-methylpyridin-4-yl)-1H-pyrrolo[3,2-c]pyridin-6-yl)-2-(oxetan-3-yl)acetamide;
1,5-dimethyl-N-(2-(pyridin-4-yl)-1H-pyrrolo[3,2-c]pyridin-6-yl)-1H-pyrazole-4-carboxamide;
(1s,3s)-3-hydroxy-N-(2-(2-methoxypyrimidin-4-yl)-1H-pyrrolo[3,2-c]pyridin-6-yl)cyclobutanecarboxamide;
N-(1-isopropyl-5-methyl-1H-pyrazol-3-yl)-2-(pyridin-4-yl)-1H-pyrrolo[3,2-c]pyridin-6-amine;
N-(2-(2-(2,2-difluoroethylamino)pyrimidin-4-yl)-1-methyl-1H-pyrrolo[3,2-c]pyridin-6-yl)-1-isopropyl-1H-pyrazole-4-carboxamide;
cyclopentyl 2-(2-methylpyridin-4-yl)-1H-pyrrolo[3,2-c]pyridin-6-ylcarbamate;
(S)—N-(2-(2-(3-fluoropropoxy)pyrimidin-4-yl)-1H-pyrrolo[3,2-c]pyridin-6-yl)-1-((tetrahydrofuran-2-yl)methyl)-1H-pyrazole-4-carboxamide;
1-isopropyl-N-(2-(2-(2,2,2-trifluoroethylamino)pyrimidin-4-yl)-1H-pyrrolo[3,2-c]pyridin-6-yl)-1H-pyrazole-4-carboxamide;
N-(2-(2-(2,2-difluoroethoxy)pyrimidin-4-yl)-1H-pyrrolo[3,2-c]pyridin-6-yl)-1-isopropyl-1H-pyrazole-4-carboxamide;
methyl 2-(2-methylpyridin-4-yl)-1H-pyrrolo[3,2-c]pyridin-6-ylcarbamate;
1-isopropyl-N-(2-(2-(3,3,3-trifluoropropoxy)pyrimidin-4-yl)-1H-pyrrolo[3,2-c]pyridin-6-yl)-1H-pyrazole-4-carboxamide;
N-(2-(2-methoxypyrimidin-4-yl)-1H-pyrrolo[3,2-c]pyridin-6-yl)bicyclo[3.1.0]hexane-6-carboxamide;
1-isopropyl-N-(2-(2-methoxypyrimidin-4-yl)-1H-pyrrolo[3,2-c]pyridin-6-yl)-1H-pyrazole-4-carboxamide;
1-isopropyl-N-(1-methyl-2-(2-(3,3,3-trifluoropropylamino)pyrimidin-4-yl)-1H-pyrrolo[3,2-c]pyridin-6-yl)-1H-pyrazole-4-carboxamide;
(S)—N-(2-(2-methoxypyrimidin-4-yl)-1H-pyrrolo[3,2-c]pyridin-6-yl)-5-methyl-1-((tetrahydrofuran-3-yl)methyl)-1H-pyrazole-4-carboxamide;
N-(2-cyano-1H-pyrrolo[3,2-c]pyridin-6-yl)cyclopropanecarboxamide;
N-(2-(pyridin-4-yl)-1H-pyrrolo[3,2-c]pyridin-6-yl)acetamide;
(S)—N-(2-(2-methylpyridin-4-yl)-1H-pyrrolo[3,2-c]pyridin-6-yl)tetrahydrofuran-2-carboxamide;
1-cyclopentyl-N-(2-(2-methoxypyrimidin-4-yl)-1H-pyrrolo[3,2-c]pyridin-6-yl)-1H-pyrazole-4-carboxamide;
(R)—N-(2-(2-methoxypyrimidin-4-yl)-1H-pyrrolo[3,2-c]pyridin-6-yl)-1-((tetrahydrofuran-3-yl)methyl)-1H-pyrazole-4-carboxamide;
1-methyl-N-(2-(2-methylpyrimidin-4-yl)-1H-pyrrolo[3,2-c]pyridin-6-yl)-1H-pyrazole-4-carboxamide;
1-isopropyl-N-(2-(2-(2,2,2-trifluoroethoxy)pyrimidin-4-yl)-1H-pyrrolo[3,2-c]pyridin-6-yl)-1H-pyrazole-4-carboxamide;
(1r,3r)-3-hydroxy-N-(2-(2-methylpyridin-4-yl)-1H-pyrrolo[3,2-c]pyridin-6-yl)cyclobutanecarboxamide;
N-(2-(2-methoxypyrimidin-4-yl)-1-methyl-1H-pyrrolo[3,2-c]pyridin-6-yl)-4-(tetrahydro-2H-pyran-4-yloxy)benzamide;
N-phenyl-2-(pyridin-3-yl)-1H-pyrrolo[3,2-c]pyridin-6-amine;
N-methyl-4-(2-(2-methylpyridin-4-yl)-1H-pyrrolo[3,2-c]pyridin-6-ylamino)benzamide;
(1s,3s)-3-methoxy-N-(2-(2-methylpyridin-4-yl)-1H-pyrrolo[3,2-c]pyridin-6-yl)cyclobutanecarboxamide;
N-(1-(2-methoxyethyl)-1H-pyrazol-4-yl)-2-(pyridin-4-yl)-1H-pyrrolo[3,2-c]pyridin-6-amine;
1-methyl-2-(1-methyl-1H-pyrazol-4-yl)-N-(4-(methylthio)phenyl)-1H-pyrrolo[3,2-c]pyridin-6-amine;
(1s,3s)-3-hydroxy-N-(2-(2-methylpyridin-4-yl)-1H-pyrrolo[3,2-c]pyridin-6-yl)cyclobutanecarboxamide;
1-methyl-N-(2-(pyridin-4-yl)-1H-pyrrolo[3,2-c]pyridin-6-yl)-1H-indazol-6-amine;
(S)—N-(2-(2-methoxypyrimidin-4-yl)-1H-pyrrolo[3,2-c]pyridin-6-yl)-5-methyl-1-((tetrahydrofuran-2-yl)methyl)-1H-pyrazole-4-carboxamide;
N-(3-methoxyphenyl)-2-(1-methyl-1H-pyrazol-4-yl)-1H-pyrrolo[3,2-c]pyridin-6-amine;
N-(2-(pyridin-3-yl)-1H-pyrrolo[3,2-c]pyridin-6-yl)acetamide;
N-(2-(2-(trifluoromethyl)pyrimidin-4-yl)-1H-pyrrolo[3,2-c]pyridin-6-yl)bicyclo[3.1.0]hexane-6-carboxamide;
1-methyl-N-(1-methyl-2-(2-(trifluoromethyl)pyrimidin-4-yl)-1H-pyrrolo[3,2-c]pyridin-6-yl)-1H-pyrazole-4-carboxamide;
N-(2-(1H-1,2,4-triazol-3-yl)-1H-pyrrolo[3,2-c]pyridin-6-yl)cyclopropanecarboxamide;
3-hydroxy-3-methyl-N-(2-(2-methylpyridin-4-yl)-1H-pyrrolo[3,2-c]pyridin-6-yl)cyclobutanecarboxamide;
N-(4-methoxyphenyl)-1-methyl-2-(1-methyl-1H-pyrazol-4-yl)-1H-pyrrolo[3,2-c]pyridin-6-amine;
N-(3-methoxyphenyl)-2-(1-methyl-1H-imidazol-5-yl)-1H-pyrrolo[3,2-c]pyridin-6-amine;
1-cyclobutyl-N-(2-(2-methylpyridin-4-yl)-1H-pyrrolo[3,2-c]pyridin-6-yl)-1H-pyrazole-4-carboxamide;
4-methoxycyclohexyl 2-(2-methylpyridin-4-yl)-1H-pyrrolo[3,2-c]pyridin-6-ylcarbamate;
2-cyclopentyl-N-(2-(pyridin-4-yl)-1H-pyrrolo[3,2-c]pyridin-6-yl)acetamide;
6-(3-(tetrahydro-2H-pyran-4-yl)-5-(1H-1,2,4-triazol-3-yl)phenylamino)-1H-pyrrolo[3,2-c]pyridine-2-carbonitrile;
1-cyclopropyl-3-(2-(2-methylpyridin-4-yl)-1H-pyrrolo[3,2-c]pyridin-6-yl)urea;
N-(2-(1-methyl-1H-pyrazol-4-yl)-1H-pyrrolo[3,2-c]pyridin-6-yl)cyclopropanecarboxamide;
N-(2-(2-methoxypyrimidin-4-yl)-1-methyl-1H-pyrrolo[3,2-c]pyridin-6-yl)bicyclo[4.1.0]heptane-7-carboxamide;
(R)—N-(2-(2-methylpyridin-4-yl)-1H-pyrrolo[3,2-c]pyridin-6-yl)tetrahydrofuran-2-carboxamide;
1-methyl-N-(2-(2-(3,3,3-trifluoropropoxy)pyrimidin-4-yl)-1H-pyrrolo[3,2-c]pyridin-6-yl)-1H-pyrazole-4-carboxamide;
1-methyl-3-(2-(2-methylpyridin-4-yl)-1H-pyrrolo[3,2-c]pyridin-6-yl)urea;
2-(4-(6-(3-(1H-1,2,4-triazol-3-yl)phenylamino)-1H-pyrrolo[3,2-c]pyridin-2-yl)pyridin-2-yl)propan-2-ol;
1-((1r,4r)-4-hydroxycyclohexyl)-N-(2-(2-(3,3,3-trifluoropropoxy)pyrimidin-4-yl)-1H-pyrrolo[3,2-c]pyridin-6-yl)-1H-pyrazole-4-carboxamide;
N-(2-(pyridin-4-yl)-1H-pyrrolo[3,2-c]pyridin-6-yl)cyclohexanecarboxamide;
3-(difluoromethyl)-1-methyl-N-(2-(2-methylpyridin-4-yl)-1H-pyrrolo[3,2-c]pyridin-6-yl)-1H-pyrazole-5-carboxamide;
1-((1r,4r)-4-hydroxycyclohexyl)-N-(2-(2-methoxypyrimidin-4-yl)-1H-pyrrolo[3,2-c]pyridin-6-yl)-1H-pyrazole-4-carboxamide;
N-(2-(2-methoxypyrimidin-4-yl)-1H-pyrrolo[3,2-c]pyridin-6-yl)-1-methyl-1H-indazole-4-carboxamide;
3,5-dimethyl-N-(2-(2-methylpyridin-4-yl)-1H-pyrrolo[3,2-c]pyridin-6-yl)isoxazole-4-carboxamide;
2-(1-methyl-1H-pyrazol-4-yl)-N-(4-(trifluoromethyl)phenyl)-1H-pyrrolo[3,2-c]pyridin-6-amine;
1-methyl-2-(1-methyl-1H-pyrazol-4-yl)-N-(tetrahydro-2H-pyran-4-yl)-1H-pyrrolo[3,2-c]pyridin-6-amine;
1-methyl-N-(2-(2-methylpyridin-4-yl)-1H-pyrrolo[3,2-c]pyridin-6-yl)-1H-pyrazole-4-carboxamide;
N-(2-(2-(2-fluoroethoxy)pyrimidin-4-yl)-1H-pyrrolo[3,2-c]pyridin-6-yl)-1-(tetrahydro-2H-pyran-4-yl)-1H-pyrazole-4-carboxamide;
1,3-dimethyl-N-(2-(2-(2,2,2-trifluoroethoxy)pyrimidin-4-yl)-1H-pyrrolo[3,2-c]pyridin-6-yl)-1H-pyrazole-4-carboxamide;
N-(2-(2-methoxypyrimidin-4-yl)-1H-pyrrolo[3,2-c]pyridin-6-yl)-2-(oxetan-3-yl)acetamide;
N-((1s,4s)-4-methoxycyclohexyl)-2-(pyridin-3-yl)-1H-pyrrolo[3,2-c]pyridin-6-amine;
N-(4-fluorophenyl)-2-(1-methyl-1H-imidazol-5-yl)-1H-pyrrolo[3,2-c]pyridin-6-amine;
N-(2-(2-ethoxypyrimidin-4-yl)-1H-pyrrolo[3,2-c]pyridin-6-yl)-1-(2-hydroxyethyl)-5-methyl-1H-pyrazole-4-carboxamide;
(R)—N-(2-(2-methoxypyrimidin-4-yl)-1H-pyrrolo[3,2-c]pyridin-6-yl)-1-((tetrahydrofuran-2-yl)methyl)-1H-pyrazole-4-carboxamide;
1-isopropyl-N-(2-(2-(methyl(2,2,2-trifluoroethyl)amino)pyrimidin-4-yl)-1H-pyrrolo[3,2-c]pyridin-6-yl)-1H-pyrazole-4-carboxamide;
N-(2-(2-ethoxypyrimidin-4-yl)-1H-pyrrolo[3,2-c]pyridin-6-yl)-1-(2-hydroxyethyl)-3-methyl-1H-pyrazole-4-carboxamide;
(S)—N-(2-(2-(2,2-difluoroethoxy)pyrimidin-4-yl)-1H-pyrrolo[3,2-c]pyridin-6-yl)-1-((tetrahydrofuran-2-yl)methyl)-1H-pyrazole-4-carboxamide;
N-(4-fluorophenyl)-1-methyl-2-(1-methyl-1H-pyrazol-4-yl)-1H-pyrrolo[3,2-c]pyridin-6-amine;
N-(2-(2-methylpyridin-4-yl)-1H-pyrrolo[3,2-c]pyridin-6-yl)-1-(tetrahydrofuran-3-yl)-1H-pyrazole-4-carboxamide;
N-(2-(2-methylpyridin-4-yl)-1H-pyrrolo[3,2-c]pyridin-6-yl)-1H-indazole-7-carboxamide;
1-methyl-N-(2-(pyridin-4-yl)-1H-pyrrolo[3,2-c]pyridin-6-yl)-1H-pyrazole-3-carboxamide;
N-(2-(2-cyanopyridin-4-yl)-1H-pyrrolo[3,2-c]pyridin-6-yl)-1-methyl-1H-pyrazole-4-carboxamide;
1,3-dimethyl-N-(1-methyl-2-(2-(2,2,2-trifluoroethylamino)pyrimidin-4-yl)-1H-pyrrolo[3,2-c]pyridin-6-yl)-1H-pyrazole-4-carboxamide;
2-(1-isopropyl-1H-pyrazol-4-yl)-1-methyl-N-(tetrahydro-2H-pyran-4-yl)-1H-pyrrolo[3,2-c]pyridin-6-amine;
1-ethyl-3-methyl-N-(2-(2-(2,2,2-trifluoroethoxy)pyrimidin-4-yl)-1H-pyrrolo[3,2-c]pyridin-6-yl)-1H-pyrazole-4-carboxamide;
N-(2-(2-ethoxypyrimidin-4-yl)-1H-pyrrolo[3,2-c]pyridin-6-yl)-1H-indazole-7-carboxamide;
(1r,3r)-N-(2-(2-ethoxypyrimidin-4-yl)-1H-pyrrolo[3,2-c]pyridin-6-yl)-3-hydroxy-3-(trifluoromethyl)cyclobutanecarboxamide;
N-(2-cyano-1-methyl-1H-pyrrolo[3,2-c]pyridin-6-yl)acetamide;
(R)—N-(2-(2-methoxypyrimidin-4-yl)-1H-pyrrolo[3,2-c]pyridin-6-yl)-3-methyl-1-((tetrahydrofuran-3-yl)methyl)-1H-pyrazole-4-carboxamide;
(1s,3s)-3-hydroxy-N-(2-(2-(trifluoromethyl)pyrimidin-4-yl)-1H-pyrrolo[3,2-c]pyridin-6-yl)cyclobutanecarboxamide;
N-(2-(6-fluoropyridin-3-yl)-1H-pyrrolo[3,2-c]pyridin-6-yl)cyclopropanecarboxamide;
N-(2-(2-ethoxypyrimidin-4-yl)-1H-pyrrolo[3,2-c]pyridin-6-yl)-3-methoxy-4-(2-methoxyethoxy)benzamide;
N-(2-(2-methoxypyrimidin-4-yl)-1H-pyrrolo[3,2-c]pyridin-6-yl)-1,3-dimethyl-1H-pyrazole-4-carboxamide;
(1s,4s)-4-(2-(pyridin-3-yl)-1H-pyrrolo[3,2-c]pyridin-6-ylamino)cyclohexanol;
3-methoxy-4-(2-methoxyethoxy)-N-(2-(2-methoxypyrimidin-4-yl)-1H-pyrrolo[3,2-c]pyridin-6-yl)benzamide;
(1r,3r)-N-(2-(2-ethoxypyrimidin-4-yl)-1H-pyrrolo[3,2-c]pyridin-6-yl)-3-hydroxycyclobutanecarboxamide;
1-methyl-N-(2-(6-(trifluoromethyl)pyrimidin-4-yl)-1H-pyrrolo[3,2-c]pyridin-6-yl)-1H-pyrazole-4-carboxamide;
N-(2-(1-methylpiperidin-4-yl)-1H-pyrrolo[3,2-c]pyridin-6-yl)cyclopropanecarboxamide;
N-(2-(2-methoxypyrimidin-4-yl)-1-methyl-1H-pyrrolo[3,2-c]pyridin-6-yl)-1-methyl-1H-pyrazole-4-carboxamide; N,1-dimethyl-N-(2-(2-(2,2,2-trifluoroethylamino)pyrimidin-4-yl)-1H-pyrrolo[3,2-c]pyridin-6-yl)-1H-pyrazole-4-carboxamide;
N-(4-methoxy-2-methylphenyl)-1-methyl-2-(1-methyl-1H-pyrazol-4-yl)-1H-pyrrolo[3,2-c]pyridin-6-amine;
1-methyl-N-(2-(tetrahydro-2H-pyran-4-yl)-1H-pyrrolo[3,2-c]pyridin-6-yl)-1H-pyrazole-4-carboxamide;
N-(2-(methoxymethyl)-1H-pyrrolo[3,2-c]pyridin-6-yl)-1-methyl-1H-pyrazole-4-carboxamide;
N-(2-(2-methylpyridin-4-yl)-1H-pyrrolo[3,2-c]pyridin-6-yl)pyrrolidine-1-carboxamide;
(1S,2S)-2-methyl-N-(2-(2-(trifluoromethyl)pyrimidin-4-yl)-1H-pyrrolo[3,2-c]pyridin-6-yl)cyclopropanecarboxamide;
(R)-2-hydroxy-N-(2-(2-methylpyridin-4-yl)-1H-pyrrolo[3,2-c]pyridin-6-yl)propanamide;
N-(2-(1H-1,2,4-triazol-3-yl)-1H-pyrrolo[3,2-c]pyridin-6-yl)-1-methyl-1H-pyrazole-4-carboxamide;
N-(1-methyl-2-phenyl-1H-pyrrolo[3,2-c]pyridin-6-yl)tetrahydro-2H-pyran-4-carboxamide;
1-methyl-2-(pyridin-3-yl)-N-(1-(tetrahydro-2H-pyran-4-yl)-1H-pyrazol-4-yl)-1H-pyrrolo[3,2-c]pyridin-6-amine;
1-methyl-N-(2-(2-methyloxazol-5-yl)-1H-pyrrolo[3,2-c]pyridin-6-yl)-1H-pyrazole-4-carboxamide;
N-(2-(5-fluoropyridin-2-yl)-1H-pyrrolo[3,2-c]pyridin-6-yl)cyclopropanecarboxamide;
N-(2-(2-methoxypyrimidin-4-yl)-1H-pyrrolo[3,2-c]pyridin-6-yl)-1H-indazole-4-carboxamide;
N-(2-(2-ethoxypyrimidin-4-yl)-1H-pyrrolo[3,2-c]pyridin-6-yl)-1-((1r,3r)-3-hydroxycyclobutyl)-1H-pyrazole-4-carboxamide; N,2-diphenyl-1H-pyrrolo[3,2-c]pyridin-6-amine;
N-(3-(1H-1,2,4-triazol-3-yl)phenyl)-2-(pyridin-3-yl)-1H-pyrrolo[3,2-c]pyridin-6-amine;
N-isobutyl-2-(pyridin-3-yl)-1H-pyrrolo[3,2-c]pyridin-6-amine;
N-((1r,4r)-4-methoxycyclohexyl)-2-(pyridin-3-yl)-1H-pyrrolo[3,2-c]pyridin-6-amine;
N-(3-(1H-1,2,4-triazol-3-yl)phenyl)-1-methyl-2-(pyridin-3-yl)-1H-pyrrolo[3,2-c]pyridin-6-amine;
N-(2-(pyridin-3-yl)-1H-pyrrolo[3,2-c]pyridin-6-yl)cyclopentanecarboxamide;
N-(2-(pyridin-4-yl)-1H-pyrrolo[3,2-c]pyridin-6-yl)benzamide;
1,3-dimethyl-N-(2-(pyridin-4-yl)-1H-pyrrolo[3,2-c]pyridin-6-yl)-1H-pyrazole-4-carboxamide;
N-(2-cyano-1-methyl-1H-pyrrolo[3,2-c]pyridin-6-yl)-1-methyl-1H-pyrazole-4-carboxamide;
N-(2-cyano-1H-pyrrolo[3,2-c]pyridin-6-yl)-1-methyl-1H-pyrazole-4-carboxamide;
1,4-dimethyl-N-(2-(pyridin-4-yl)-1H-pyrrolo[3,2-c]pyridin-6-yl)-1H-pyrazole-3-carboxamide;
1,5-dimethyl-N-(2-(pyridin-4-yl)-1H-pyrrolo[3,2-c]pyridin-6-yl)-1H-pyrazole-3-carboxamide;
N-(2-(3-fluoro-2-methylpyridin-4-yl)-1H-pyrrolo[3,2-c]pyridin-6-yl)-1-methyl-1H-pyrazole-4-carboxamide;
1-methyl-N-(2-(2-(trifluoromethyl)pyridin-4-yl)-1H-pyrrolo[3,2-c]pyridin-6-yl)-1H-pyrazole-4-carboxamide;
1-methyl-N-(2-methyl-1H-pyrrolo[3,2-c]pyridin-6-yl)-1H-pyrazole-4-carboxamide;
N-(2-(2-methoxypyridin-4-yl)-1H-pyrrolo[3,2-c]pyridin-6-yl)-1-methyl-1H-pyrazole-4-carboxamide;
1-methyl-N-(2-(2-(trifluoromethyl)pyrimidin-4-yl)-1H-pyrrolo[3,2-c]pyridin-6-yl)-1H-pyrazole-4-carboxamide;
1-methyl-N-(2-(2-methylpyridin-4-yl)-1H-pyrrolo[3,2-c]pyridin-6-yl)-1H-indazole-4-carboxamide;
N-(2-(2-methylpyridin-4-yl)-1H-pyrrolo[3,2-c]pyridin-6-yl)-1H-indazole-4-carboxamide;
5-methyl-N-(2-(2-methylpyridin-4-yl)-1H-pyrrolo[3,2-c]pyridin-6-yl)oxazole-4-carboxamide;
1,1-dimethyl-3-(2-(2-methylpyridin-4-yl)-1H-pyrrolo[3,2-c]pyridin-6-yl)urea;
N-(2-(2-methoxypyrimidin-4-yl)-1H-pyrrolo[3,2-c]pyridin-6-yl)-1,5-dimethyl-1H-pyrazole-3-carboxamide;
N-(2-(2-ethoxypyrimidin-4-yl)-1H-pyrrolo[3,2-c]pyridin-6-yl)-1-methyl-1H-pyrazole-4-carboxamide;
1-methyl-N-(2-(2-methylpyridin-4-yl)-1H-pyrrolo[3,2-c]pyridin-6-yl)-5-(trifluoromethyl)-1H-pyrazole-3-carboxamide;
3-methoxy-4-(2-methoxyethoxy)-N-(2-(2-methylpyridin-4-yl)-1H-pyrrolo[3,2-c]pyridin-6-yl)benzamide;
3,4-dimethoxy-N-(2-(2-methylpyridin-4-yl)-1H-pyrrolo[3,2-c]pyridin-6-yl)benzamide;
N-(2-(2-methoxypyrimidin-4-yl)-1H-pyrrolo[3,2-c]pyridin-6-yl)pivalamide;
N-(2-(2-methylpyridin-4-yl)-1H-pyrrolo[3,2-c]pyridin-6-yl)bicyclo[1.1.1]pentane-1-carboxamide;
1-methyl-N-(2-(4-(trifluoromethyl)pyridin-2-yl)-1H-pyrrolo[3,2-c]pyridin-6-yl)-1H-pyrazole-4-carboxamide;
N-(2-(4-cyanopyridin-2-yl)-1H-pyrrolo[3,2-c]pyridin-6-yl)-1-methyl-1H-pyrazole-4-carboxamide;
N-(2-(2-methylpyridin-4-yl)-1H-pyrrolo[3,2-c]pyridin-6-yl)pivalamide;
N-(1-methyl-2-(2-(trifluoromethyl)pyrimidin-4-yl)-1H-pyrrolo[3,2-c]pyridin-6-yl)cyclopropanecarboxamide;
N-(2-(5-cyanopyrazin-2-yl)-1H-pyrrolo[3,2-c]pyridin-6-yl)-1-methyl-1H-pyrazole-4-carboxamide;
1-methyl-N-(2-(5-(trifluoromethyl)pyrazin-2-yl)-1H-pyrrolo[3,2-c]pyridin-6-yl)-1H-pyrazole-4-carboxamide;
1,5-dimethyl-N-(2-(2-(2,2,2-trifluoroethylamino)pyridin-4-yl)-1H-pyrrolo[3,2-c]pyridin-6-yl)-1H-pyrazole-3-carboxamide;
1-methyl-N-(2-(6-(trifluoromethyl)pyridin-3-yl)-1H-pyrrolo[3,2-c]pyridin-6-yl)-1H-pyrazole-4-carboxamide;
(S)-2-hydroxy-N-(2-(2-methylpyridin-4-yl)-1H-pyrrolo[3,2-c]pyridin-6-yl)propanamide;
1-methyl-N-(2-(2-(2,2,2-trifluoroethoxy)pyrimidin-4-yl)-1H-pyrrolo[3,2-c]pyridin-6-yl)-1H-pyrazole-4-carboxamide;
1-isopropyl-N-(2-(2-methoxypyrimidin-4-yl)-1-methyl-1H-pyrrolo[3,2-c]pyridin-6-yl)-1H-pyrazole-4-carboxamide;
N-(2-(2-(2,2-difluoroethoxy)pyrimidin-4-yl)-1H-pyrrolo[3,2-c]pyridin-6-yl)-1-methyl-1H-pyrazole-4-carboxamide;
N-(2-(2-isobutoxypyrimidin-4-yl)-1H-pyrrolo[3,2-c]pyridin-6-yl)-1-methyl-1H-pyrazole-4-carboxamide;
1-methyl-N-(2-(2-propoxypyrimidin-4-yl)-1H-pyrrolo[3,2-c]pyridin-6-yl)-1H-pyrazole-4-carboxamide;
N-(2-(2-(cyclobutylmethoxy)pyrimidin-4-yl)-1H-pyrrolo[3,2-c]pyridin-6-yl)-1-methyl-1H-pyrazole-4-carboxamide;
(R)-1-methyl-N-(2-(2-(1,1,1-trifluoropropan-2-yloxy)pyrimidin-4-yl)-1H-pyrrolo[3,2-c]pyridin-6-yl)-1H-pyrazole-4-carboxamide;
N-(2-(2-methoxy-6-methylpyrimidin-4-yl)-1H-pyrrolo[3,2-c]pyridin-6-yl)-1-methyl-1H-pyrazole-4-carboxamide;
N-(2-(2-ethoxypyrimidin-4-yl)-1-isobutyl-1H-pyrrolo[3,2-c]pyridin-6-yl)-1-methyl-1H-pyrazole-4-carboxamide;
N-(2-(2-(2,2-difluoroethoxy)pyrimidin-4-yl)-1H-pyrrolo[3,2-c]pyridin-6-yl)-1,3-dimethyl-1H-pyrazole-4-carboxamide;
N-(2-(2-(2,2-difluoroethoxy)pyrimidin-4-yl)-1H-pyrrolo[3,2-c]pyridin-6-yl)-1-ethyl-3-methyl-1H-pyrazole-4-carboxamide;
1-methyl-N-(2-(2-(neopentyloxy)pyrimidin-4-yl)-1H-pyrrolo[3,2-c]pyridin-6-yl)-1H-pyrazole-4-carboxamide;
N-(2-(2-methoxypyrimidin-4-yl)-1H-pyrrolo[3,2-c]pyridin-6-yl)-4-morpholinobenzamide;
N-(2-(2-ethoxypyrimidin-4-yl)-1H-pyrrolo[3,2-c]pyridin-6-yl)-3,4-dimethoxybenzamide;
(S)—N-(2-(2-ethoxypyrimidin-4-yl)-1H-pyrrolo[3,2-c]pyridin-6-yl)-3-methyl-1-((tetrahydrofuran-2-yl)methyl)-1H-pyrazole-4-carboxamide;
N-(2-(2-(2,2-difluoroethoxy)pyrimidin-4-yl)-1H-pyrrolo[3,2-c]pyridin-6-yl)-1-((1s,4s)-4-hydroxycyclohexyl)-1H-pyrazole-4-carboxamide;
N-(2-(2-(3-hydroxycyclobutoxy)pyrimidin-4-yl)-1H-pyrrolo[3,2-c]pyridin-6-yl)-1-methyl-1H-pyrazole-4-carboxamide;
N-(2-(2-((1s,4s)-4-hydroxycyclohexyloxy)pyrimidin-4-yl)-1H-pyrrolo[3,2-c]pyridin-6-yl)-1-methyl-1H-pyrazole-4-carboxamide;
(S)—N-(2-(2-methoxypyrimidin-4-yl)-1H-pyrrolo[3,2-c]pyridin-6-yl)-3-methyl-1-((tetrahydrofuran-3-yl)methyl)-1H-pyrazole-4-carboxamide;
N-(2-methyl-1H-pyrrolo[3,2-c]pyridin-6-yl)cyclopropanecarboxamide;
1-methyl-N-(2-(6-methylpyrimidin-4-yl)-1H-pyrrolo[3,2-c]pyridin-6-yl)-1H-pyrazole-4-carboxamide;
N-(1-isopropyl-2-(2-methylpyridin-4-yl)-1H-pyrrolo[3,2-c]pyridin-6-yl)cyclopropanecarboxamide;
N-(2-(1H-imidazol-5-yl)-1H-pyrrolo[3,2-c]pyridin-6-yl)cyclopropanecarboxamide;
1-methyl-N-(2-(1-methyl-1H-pyrazol-4-yl)-1H-pyrrolo[3,2-c]pyridin-6-yl)-1H-pyrazole-4-carboxamide;
1-methyl-N-(2-(pyridin-3-yl)-1H-pyrrolo[3,2-c]pyridin-6-yl)-1H-pyrazole-4-carboxamide;
N-(1-(2-(dimethylamino)ethyl)-2-(2-methylpyridin-4-yl)-1H-pyrrolo[3,2-c]pyridin-6-yl)cyclopropanecarboxamide;
N-(2-(5-fluoropyridin-2-yl)-1H-pyrrolo[3,2-c]pyridin-6-yl)-1-methyl-1H-pyrazole-4-carboxamide;
N-(2-(2-methyloxazol-4-yl)-1H-pyrrolo[3,2-c]pyridin-6-yl)cyclopropanecarboxamide;
1-methyl-N-(2-(1-methylpiperidin-4-yl)-1H-pyrrolo[3,2-c]pyridin-6-yl)-1H-pyrazole-4-carboxamide;
N-(3-methyl-5-(1H-1,2,4-triazol-1-yl)phenyl)-2-(pyridin-3-yl)-1H-pyrrolo[3,2-c]pyridin-6-amine;
N-(1-isopropyl-2-(2-(2,2,2-trifluoroethylamino)pyrimidin-4-yl)-1H-pyrrolo[3,2-c]pyridin-6-yl)-1-methyl-1H-pyrazole-4-carboxamide;
4-(6-(3-methyl-5-(1H-1,2,4-triazol-1-yl)phenylamino)-1H-pyrrolo[3,2-c]pyridin-2-yl)picolinonitrile;
N-(2-(4-fluoro-3-methylphenyl)-1H-pyrrolo[3,2-c]pyridin-6-yl)cyclopropanecarboxamide;
1-methyl-N-(2-(trifluoromethyl)-1H-pyrrolo[3,2-c]pyridin-6-yl)-1H-pyrazole-4-carboxamide;
N-(1-methyl-2-(1-methyl-1H-imidazol-4-yl)-1H-pyrrolo[3,2-c]pyridin-6-yl)cyclopropanecarboxamide;
N-(1-(2-hydroxyethyl)-2-(2-methoxypyrimidin-4-yl)-1H-pyrrolo[3,2-c]pyridin-6-yl)cyclopropanecarboxamide;
N-(1-ethyl-2-(2-methoxypyrimidin-4-yl)-1H-pyrrolo[3,2-c]pyridin-6-yl)cyclopropanecarboxamide;
N-(1-methyl-2-(1-methyl-1H-pyrazol-3-yl)-1H-pyrrolo[3,2-c]pyridin-6-yl)cyclopropanecarboxamide;
N-(1-methyl-2-m-tolyl-1H-pyrrolo[3,2-c]pyridin-6-yl)cyclopropanecarboxamide;
N-(1-methyl-2-(5-methylpyridin-2-yl)-1H-pyrrolo[3,2-c]pyridin-6-yl)cyclopropanecarboxamide;
1-methyl-2-(1-methyl-1H-imidazol-4-yl)-N-(tetrahydro-2H-pyran-4-yl)-1H-pyrrolo[3,2-c]pyridin-6-amine;
2-(pyridin-2-yl)-N-(tetrahydro-2H-pyran-4-yl)-1H-pyrrolo[3,2-c]pyridin-6-amine;
2-(4-methylpyridin-2-yl)-N-(tetrahydro-2H-pyran-4-yl)-1H-pyrrolo[3,2-c]pyridin-6-amine;
N-(tetrahydro-2H-pyran-4-yl)-2-p-tolyl-1H-pyrrolo[3,2-c]pyridin-6-amine;
N-(tetrahydro-2H-pyran-4-yl)-2-m-tolyl-1H-pyrrolo[3,2-c]pyridin-6-amine;
2-(pyridin-4-yl)-N-(tetrahydro-2H-pyran-4-yl)-1H-pyrrolo[3,2-c]pyridin-6-amine;
2-(1-methyl-1H-pyrazol-4-yl)-N-(tetrahydro-2H-pyran-4-yl)-1H-pyrrolo[3,2-c]pyridin-6-amine;
(S)-2-(2-methylpyridin-4-yl)-N-(tetrahydro-2H-pyran-3-yl)-1H-pyrrolo[3,2-c]pyridin-6-amine;
(R)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-o-tolyl-1H-pyrrolo[3,2-c]pyridin-6-amine;
1-methyl-N-(tetrahydro-2H-pyran-4-yl)-2-(2-(2,2,2-trifluoroethylamino)pyrimidin-4-yl)-1H-pyrrolo[3,2-c]pyridin-6-amine;
2-isopropyl-N-(tetrahydro-2H-pyran-4-yl)-1H-pyrrolo[3,2-c]pyridin-6-amine;
N-(tetrahydro-2H-pyran-4-yl)-2-(6-(2,2,2-trifluoroethylamino)pyrimidin-4-yl)-1H-pyrrolo[3,2-c]pyridin-6-amine;
2-ethyl-N-(tetrahydro-2H-pyran-4-yl)-1H-pyrrolo[3,2-c]pyridin-6-amine;
3-methyl-2-(2-methylpyridin-4-yl)-N-(tetrahydro-2H-pyran-4-yl)-1H-pyrrolo[3,2-c]pyridin-6-amine;
N-(tetrahydro-2H-pyran-4-yl)-2-(2-(2,2,2-trifluoroethylamino)pyridin-4-yl)-1H-pyrrolo[3,2-c]pyridin-6-amine;
2-cyclohexyl-N-(tetrahydro-2H-pyran-4-yl)-1H-pyrrolo[3,2-c]pyridin-6-amine;
1-methyl-N-(tetrahydro-2H-pyran-4-yl)-2-(6-(2,2,2-trifluoroethylamino)pyrimidin-4-yl)-1H-pyrrolo[3,2-c]pyridin-6-amine;
(R)—N-(tetrahydrofuran-3-yl)-2-(2-(2,2,2-trifluoroethylamino)pyrimidin-4-yl)-1H-pyrrolo[3,2-c]pyridin-6-amine;
(S)-3-methyl-2-(2-methylpyridin-4-yl)-N-(tetrahydrofuran-3-yl)-1H-pyrrolo[3,2-c]pyridin-6-amine;
(R)-3-methyl-2-(2-methylpyridin-4-yl)-N-(tetrahydrofuran-3-yl)-1H-pyrrolo[3,2-c]pyridin-6-amine;
N-cyclohexyl-3-methyl-2-(2-methylpyridin-4-yl)-1H-pyrrolo[3,2-c]pyridin-6-amine;
2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
1-methyl-2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2-(pyrrolidin-1-yl)pyrimidin-4-yl)-N-(tetrahydro-2H-pyran-4-yl)-1H-pyrrolo[3,2-c]pyridin-6-amine;
N-cyclobutyl-2-(2-(2,2,2-trifluoroethylamino)pyrimidin-4-yl)-1H-pyrrolo[3,2-c]pyridin-6-amine;
(S)—N-(tetrahydrofuran-3-yl)-2-(2-(2,2,2-trifluoroethylamino)pyrimidin-4-yl)-1H-pyrrolo[3,2-c]pyridin-6-amine;
2-(2-methylpyridin-4-yl)-N-(tetrahydro-2H-pyran-4-yl)-3-(trifluoromethyl)-1H-pyrrolo[3,2-c]pyridin-6-amine;
N-(tetrahydro-2H-pyran-4-yl)-3-(trifluoromethyl)-1H-pyrrolo[3,2-c]pyridin-6-amine;
2-(2-ethoxypyrimidin-4-yl)-N-(tetrahydro-2H-pyran-4-yl)-1H-pyrrolo[3,2-c]pyridin-6-amine;
1-methyl-2-(2-methylpyrimidin-4-yl)-N-(tetrahydro-2H-pyran-4-yl)-1H-pyrrolo[3,2-c]pyridin-6-amine;
(S)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(2,2,2-trifluoroethoxy)pyrimidin-4-yl)-1H-pyrrolo[3,2-c]pyridin-6-amine;
2-(2-(methyl(2,2,2-trifluoroethyl)amino)pyridin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(2-(2,2,2-trifluoroethylamino)pyridin-4-yl)-1H-pyrrolo[3,2-c]pyridin-6-amine;
N-isopropyl-1-methyl-2-(2-(2,2,2-trifluoroethylamino)pyrimidin-4-yl)-1H-pyrrolo[3,2-c]pyridin-6-amine;
N-tert-butyl-1-methyl-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(3,3,3-trifluoropropyl)pyrimidin-4-yl)-1H-pyrrolo[3,2-c]pyridin-6-amine;
(R)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
2-(2-ethylpyrimidin-4-yl)-1-methyl-N-(tetrahydro-2H-pyran-4-yl)-1H-pyrrolo[3,2-c]pyridin-6-amine;
2-(6-methoxypyrimidin-4-yl)-1-methyl-N-(tetrahydro-2H-pyran-4-yl)-1H-pyrrolo[3,2-c]pyridin-6-amine;
2-(6-ethylpyrimidin-4-yl)-1-methyl-N-(tetrahydro-2H-pyran-4-yl)-1H-pyrrolo[3,2-c]pyridin-6-amine; N,1-dimethyl-2-(2-(2,2,2-trifluoroethylamino)pyrimidin-4-yl)-1H-pyrrolo[3,2-c]pyridin-6-amine;
2-(2-isopropylpyrimidin-4-yl)-1-methyl-N-(tetrahydro-2H-pyran-4-yl)-1H-pyrrolo[3,2-c]pyridin-6-amine;
1-methyl-N-(tetrahydro-2H-pyran-4-yl)-2-(6-(trifluoromethyl)pyrimidin-4-yl)-1H-pyrrolo[3,2-c]pyridin-6-amine;
2-(2,6-dimethylpyrimidin-4-yl)-N-(tetrahydro-2H-pyran-4-yl)-1H-pyrrolo[3,2-c]pyridin-6-amine;
2-(6-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(6-isopropylpyrimidin-4-yl)-1-methyl-N-(tetrahydro-2H-pyran-4-yl)-1H-pyrrolo[3,2-c]pyridin-6-amine;
1-methyl-N-(tetrahydro-2H-pyran-4-yl)-2-(2-(trifluoromethyl)pyrimidin-4-yl)-1H-pyrrolo[3,2-c]pyridin-6-amine;
   and pharmaceutically acceptable salts, tautomers, isotopologues, or stereoisomers thereof.

In certain embodiments, the compound is chosen from 2-(2-methylpyridin-4-yl)-N-(tetrahydro-2H-pyran-4-yl)-1H-pyrrolo[3,2-c]pyridin-6-amine;
2-(6-methylpyrimidin-4-yl)-N-(tetrahydro-2H-pyran-4-yl)-1H-pyrrolo[3,2-c]pyridin-6-amine;
1-methyl-2-(6-methylpyrimidin-4-yl)-N-(tetrahydro-2H-pyran-4-yl)-1H-pyrrolo[3,2-c]pyridin-6-amine;
(R)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-(2-(2,2,2-trifluoroethylamino)pyridin-4-yl)-1H-pyrrolo[3,2-c]pyridin-6-amine;
N-(2-(2-methylpyridin-4-yl)-1H-pyrrolo[3,2-c]pyridin-6-yl)cyclopropanecarboxamide;
(R)-1-methyl-N-(2-(2-(1,1,1-trifluoropropan-2-ylamino)pyrimidin-4-yl)-1H-pyrrolo[3,2-c]pyridin-6-yl)-1H-pyrazole-4-carboxamide;
N-(cyclopropylmethyl)-2-(2-methylpyridin-4-yl)-1H-pyrrolo[3,2-c]pyridin-6-amine;
4-(6-(1-methyl-1H-pyrazol-4-ylamino)-1H-pyrrolo[3,2-c]pyridin-2-yl)picolinonitrile N-(tetrahydro-2H-pyran-4-yl)-2-(2-(2,2,2-trifluoroethylamino)pyrimidin-4-yl)-1H-pyrrolo[3,2-c]pyridin-6-amine;
4-(6-(3-(tetrahydro-2H-pyran-4-yl)phenylamino)-1H-pyrrolo[3,2-c]pyridin-2-yl)picolinonitrile;
N-(3-methyl-5-(1H-1,2,4-triazol-1-yl)phenyl)-2-(2-(2,2,2-trifluoroethylamino)pyrimidin-4-yl)-1H-pyrrolo[3,2-c]pyridin-6-amine;
(R)-2-(2-methylpyridin-4-yl)-N-(tetrahydrofuran-3-yl)-1H-pyrrolo[3,2-c]pyridin-6-amine;
2-(2-methoxypyrimidin-4-yl)-N-(tetrahydro-2H-pyran-4-yl)-1H-pyrrolo[3,2-c]pyridin-6-amine;
1-methyl-2-(2-methylpyridin-4-yl)-N-(tetrahydro-2H-pyran-4-yl)-1H-pyrrolo[3,2-c]pyridin-6-amine;
2-(2-methylpyrimidin-4-yl)-N-(tetrahydro-2H-pyran-4-yl)-1H-pyrrolo[3,2-c]pyridin-6-amine;
2-(2-methylpyridin-4-yl)-N-(4-methyltetrahydro-2H-pyran-4-yl)-1H-pyrrolo[3,2-c]pyridin-6-amine;
(S)-2-(2-methylpyridin-4-yl)-N-(tetrahydrofuran-3-yl)-1H-pyrrolo[3,2-c]pyridin-6-amine;
(S)-2-(2-methylpyridin-4-yl)-N-(tetrahydrofuran-3-yl)-1H-pyrrolo[3,2-c]pyridin-6-amine;
2-(pyridin-4-yl)-N-(tetrahydro-2H-pyran-4-yl)-1H-pyrrolo[3,2-c]pyridin-6-amine;
(S)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-o-tolyl-1H-pyrrolo[3,2-c]pyridin-6-amine;
1-methyl-N-(tetrahydro-2H-pyran-4-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(6-(2,2,2-trifluoroethylamino)pyrimidin-4-yl)-1H-pyrrolo[3,2-c]pyridin-6-amine;
2-cyclohexyl-N-(tetrahydro-2H-pyran-4-yl)-1H-pyrrolo[3,2-c]pyridin-6-amine;
1-methyl-N-(tetrahydro-2H-pyran-4-yl)-2-(6-(2,2,2-trifluoroethylamino)pyrimidin-4-yl)-1H-pyrrolo[3,2-c]pyridin-6-amine;
(R)—N-(tetrahydrofuran-3-yl)-2-(2-(2,2,2-trifluoroethylamino)pyrimidin-4-yl)-1H-pyrrolo[3,2-c]pyridin-6-amine;
2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
1-methyl-2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2-(pyrrolidin-1-yl)pyrimidin-4-yl)-N-(tetrahydro-2H-pyran-4-yl)-1H-pyrrolo[3,2-c]pyridin-6-amine;
N-cyclobutyl-2-(2-(2,2,2-trifluoroethylamino)pyrimidin-4-yl)-1H-pyrrolo[3,2-c]pyridin-6-amine;
(S)—N-(tetrahydrofuran-3-yl)-2-(2-(2,2,2-trifluoroethylamino)pyrimidin-4-yl)-1H-pyrrolo[3,2-c]pyridin-6-amine;
2-(2-ethoxypyrimidin-4-yl)-N-(tetrahydro-2H-pyran-4-yl)-1H-pyrrolo[3,2-c]pyridin-6-amine;
1-methyl-2-(2-methylpyrimidin-4-yl)-N-(tetrahydro-2H-pyran-4-yl)-1H-pyrrolo[3,2-c]pyridin-6-amine;
(S)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(2,2,2-trifluoroethoxy)pyrimidin-4-yl)-1H-pyrrolo[3,2-c]pyridin-6-amine;
2-(2-(methyl(2,2,2-trifluoroethyl)amino)pyridin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(2-(2,2,2-trifluoroethylamino)pyridin-4-yl)-1H-pyrrolo[3,2-c]pyridin-6-amine;
N-isopropyl-1-methyl-2-(2-(2,2,2-trifluoroethylamino)pyrimidin-4-yl)-1H-pyrrolo[3,2-c]pyridin-6-amine;
N-tert-butyl-1-methyl-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(3,3,3-trifluoropropyl)pyrimidin-4-yl)-1H-pyrrolo[3,2-c]pyridin-6-amine;
(R)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
2-(6-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2,6-dimethylpyrimidin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(6-(trifluoromethyl)pyrimidin-4-yl)-1H-pyrrolo[3,2-c]pyridin-6-amine;
2-(2-isopropylpyrimidin-4-yl)-1-methyl-N-(tetrahydro-2H-pyran-4-yl)-1H-pyrrolo[3,2-c]pyridin-6-amine; N,1-dimethyl-2-(2-(2,2,2-trifluoroethylamino)pyrimidin-4-yl)-1H-pyrrolo[3,2-c]pyridin-6-amine;
2-(6-ethylpyrimidin-4-yl)-1-methyl-N-(tetrahydro-2H-pyran-4-yl)-1H-pyrrolo[3,2-c]pyridin-6-amine;
2-(6-methoxypyrimidin-4-yl)-1-methyl-N-(tetrahydro-2H-pyran-4-yl)-1H-pyrrolo[3,2-c]pyridin-6-amine;
2-(2-ethylpyrimidin-4-yl)-1-methyl-N-(tetrahydro-2H-pyran-4-yl)-1H-pyrrolo[3,2-c]pyridin-6-amine; and and pharmaceutically acceptable salts, tautomers, isotopologues, or stereoisomers thereof.

In one embodiment, the compound is chosen from 2-(2-methylpyridin-4-yl)-N-(tetrahydro-2H-pyran-4-yl)-1H-pyrrolo[3,2-c]pyridin-6-amine;
2-(6-methylpyrimidin-4-yl)-N-(tetrahydro-2H-pyran-4-yl)-1H-pyrrolo[3,2-c]pyridin-6-amine;
1-methyl-2-(6-methylpyrimidin-4-yl)-N-(tetrahydro-2H-pyran-4-yl)-1H-pyrrolo[3,2-c]pyridin-6-amine;
(R)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-(2-(2,2,2-trifluoroethylamino)pyridin-4-yl)-1H-pyrrolo[3,2-c]pyridin-6-amine;
N-(2-(2-methylpyridin-4-yl)-1H-pyrrolo[3,2-c]pyridin-6-yl)cyclopropanecarboxamide;
(R)-1-methyl-N-(2-(2-(1,1,1-trifluoropropan-2-ylamino)pyrimidin-4-yl)-1H-pyrrolo[3,2-c]pyridin-6-yl)-1H-pyrazole-4-carboxamide;
   and pharmaceutically acceptable salts, tautomers, isotopologues, or stereoisomers thereof.

In another embodiment, the compound is chosen from
N-(cyclopropylmethyl)-2-(2-methylpyridin-4-yl)-1H-pyrrolo[3,2-c]pyridin-6-amine;
4-(6-(1-methyl-1H-pyrazol-4-ylamino)-1H-pyrrolo[3,2-c]pyridin-2-yl)picolinonitrile;
N-(tetrahydro-2H-pyran-4-yl)-2-(2-(2,2,2-trifluoroethylamino)pyrimidin-4-yl)-1H-pyrrolo[3,2-c]pyridin-6-amine;
4-(6-(3-(tetrahydro-2H-pyran-4-yl)phenylamino)-1H-pyrrolo[3,2-c]pyridin-2-yl)picolinonitrile;
N-(3-methyl-5-(1H-1,2,4-triazol-1-yl)phenyl)-2-(2-(2,2,2-trifluoroethylamino)pyrimidin-4-yl)-1H-pyrrolo[3,2-c]pyridin-6-amine;
(R)-2-(2-methylpyridin-4-yl)-N-(tetrahydrofuran-3-yl)-1H-pyrrolo[3,2-c]pyridin-6-amine;
2-(2-methoxypyrimidin-4-yl)-N-(tetrahydro-2H-pyran-4-yl)-1H-pyrrolo[3,2-c]pyridin-6-amine;
1-methyl-2-(2-methylpyridin-4-yl)-N-(tetrahydro-2H-pyran-4-yl)-1H-pyrrolo[3,2-c]pyridin-6-amine;
2-(2-methylpyrimidin-4-yl)-N-(tetrahydro-2H-pyran-4-yl)-1H-pyrrolo[3,2-c]pyridin-6-amine;
2-(2-methylpyridin-4-yl)-N-(4-methyltetrahydro-2H-pyran-4-yl)-1H-pyrrolo[3,2-c]pyridin-6-amine;
(S)-2-(2-methylpyridin-4-yl)-N-(tetrahydrofuran-3-yl)-1H-pyrrolo[3,2-c]pyridin-6-amine;
(S)-2-(2-methylpyridin-4-yl)-N-(tetrahydrofuran-3-yl)-1H-pyrrolo[3,2-c]pyridin-6-amine;
2-(pyridin-4-yl)-N-(tetrahydro-2H-pyran-4-yl)-1H-pyrrolo[3,2-c]pyridin-6-amine;
(S)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-o-tolyl-1H-pyrrolo[3,2-c]pyridin-6-amine;
1-methyl-N-(tetrahydro-2H-pyran-4-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(6-(2,2,2-trifluoroethylamino)pyrimidin-4-yl)-1H-pyrrolo[3,2-c]pyridin-6-amine;
2-cyclohexyl-N-(tetrahydro-2H-pyran-4-yl)-1H-pyrrolo[3,2-c]pyridin-6-amine;
1-methyl-N-(tetrahydro-2H-pyran-4-yl)-2-(6-(2,2,2-trifluoroethylamino)pyrimidin-4-yl)-1H-pyrrolo[3,2-c]pyridin-6-amine;
(R)—N-(tetrahydrofuran-3-yl)-2-(2-(2,2,2-trifluoroethylamino)pyrimidin-4-yl)-1H-pyrrolo[3,2-c]pyridin-6-amine;
2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
1-methyl-2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2-(pyrrolidin-1-yl)pyrimidin-4-yl)-N-(tetrahydro-2H-pyran-4-yl)-1H-pyrrolo[3,2-c]pyridin-6-amine;
N-cyclobutyl-2-(2-(2,2,2-trifluoroethylamino)pyrimidin-4-yl)-1H-pyrrolo[3,2-c]pyridin-6-amine;
(S)—N-(tetrahydrofuran-3-yl)-2-(2-(2,2,2-trifluoroethylamino)pyrimidin-4-yl)-1H-pyrrolo[3,2-c]pyridin-6-amine;
2-(2-ethoxypyrimidin-4-yl)-N-(tetrahydro-2H-pyran-4-yl)-1H-pyrrolo[3,2-c]pyridin-6-amine;
1-methyl-2-(2-methylpyrimidin-4-yl)-N-(tetrahydro-2H-pyran-4-yl)-1H-pyrrolo[3,2-c]pyridin-6-amine;
(S)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(2,2,2-trifluoroethoxy)pyrimidin-4-yl)-1H-pyrrolo[3,2-c]pyridin-6-amine;
2-(2-(methyl(2,2,2-trifluoroethyl)amino)pyridin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(2-(2,2,2-trifluoroethylamino)pyridin-4-yl)-1H-pyrrolo[3,2-c]pyridin-6-amine;
N-isopropyl-1-methyl-2-(2-(2,2,2-trifluoroethylamino)pyrimidin-4-yl)-1H-pyrrolo[3,2-c]pyridin-6-amine;
N-tert-butyl-1-methyl-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(3,3,3-trifluoropropyl)pyrimidin-4-yl)-1H-pyrrolo[3,2-c]pyridin-6-amine;
(R)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
2-(6-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2,6-dimethylpyrimidin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(6-(trifluoromethyl)pyrimidin-4-yl)-1H-pyrrolo[3,2-c]pyridin-6-amine;
2-(2-isopropylpyrimidin-4-yl)-1-methyl-N-(tetrahydro-2H-pyran-4-yl)-1H-pyrrolo[3,2-c]pyridin-6-amine; N,1-dimethyl-2-(2-(2,2,2-trifluoroethylamino)pyrimidin-4-yl)-1H-pyrrolo[3,2-c]pyridin-6-amine;
2-(6-ethylpyrimidin-4-yl)-1-methyl-N-(tetrahydro-2H-pyran-4-yl)-1H-pyrrolo[3,2-c]pyridin-6-amine;
2-(6-methoxypyrimidin-4-yl)-1-methyl-N-(tetrahydro-2H-pyran-4-yl)-1H-pyrrolo[3,2-c]pyridin-6-amine;
2-(2-ethylpyrimidin-4-yl)-1-methyl-N-(tetrahydro-2H-pyran-4-yl)-1H-pyrrolo[3,2-c]pyridin-6-amine;
   and pharmaceutically acceptable salts, tautomers, isotopologues, or stereoisomers thereof

Further embodiments provided herein include combinations of at least one of the particular embodiments set forth above.

Representative compounds of Formula I, Formula II and Formula III are set forth in Table 1.

### METHODS OF USE

The Heterocyclic Compounds, including compounds of Formula I, Formula II, Formula III and Table 1, have utility as pharmaceuticals to treat, prevent or improve conditions in animals and humans. The Heterocyclic Compounds provided herein have utility for use in the treatment or prevention of diseases, disorders or conditions disclosed herein. The Heterocyclic Compounds provided herein have utility for use in the inhibition of certain activity disclosed herein.

In one aspect, provided herein is a method of treating cryptosporidiosis. In certain embodiments, a compound as described herein is used in human medical therapy, particularly in the treatment of cryptosporidiosis. In certain embodiments, a compound as provided herein is used in animal medical therapy, particularly in the treatment of cryptosporidiosis. In certain embodiments, the method includes administering a therapeutically effective amount of a compound as described to a subject having a disease caused by cryptosporidiosis.

In one embodiment, provided herein is a method for the treatment or prevention of cryptosporidiosis, the method comprising administering to a subject in need thereof an effective amount of a Heterocyclic Compound.

In one aspect, provided herein is a method of inhibiting a parasite or parasitic activity. In certain embodiments, a compound as described herein is used in human medical therapy, particularly in inhibiting a parasite or parasitic activity. In certain embodiments, a compound as provided herein is used in animal medical therapy, particularly in inhibiting a parasite or parasitic activity. In certain embodiments, the method includes administering a therapeutically effective amount of a compound as described to a subject for inhibiting a parasite or parasitic activity.

In one embodiment, provided herein is a method for inhibiting a parasite or parasitic activity in a subject, the methods comprising administering to a subject in need thereof an effective amount of a Heterocyclic Compound. In certain embodiments, the parasite is Cryptosporidium parvum. In certain embodiments, the parasite is Cryptosporidium hominis.

In certain aspects, the present methods comprise a step of administering a Heterocyclic Compound to a subject. In certain embodiments, the methods comprise administering a Heterocyclic Compound to a subject for no more than fourteen (14) days. In certain embodiments, the methods comprise administering a Heterocyclic Compound to a subject for no more than seven (7) days.

In certain embodiments, the subject is in need of treatment for cryptosporidiosis. In certain embodiments, the subject has cryptosporidiosis. In certain embodiments, the subject is an animal. In certain embodiments, the subject is a cow. In certain embodiments, the subject is a domestic animal. In certain embodiments, the subject is a dog.

In certain embodiments, the subject is in need of inhibition of a parasite or parasitic activity. In certain embodiments, the subject has a parasite In certain embodiments, the subject is an animal. In certain embodiments, the subject is a cow. In certain embodiments, the subject is a domestic animal. In certain embodiments, the subject is a dog.

As discussed herein, compounds provided herein are useful for treating and preventing certain diseases and disorders, as well as inhibiting a parasite or parasitic activity, in humans and animals. In certain embodiments, a Heterocyclic Compound is used to treat cryptosporidiosis. In other embodiments, a Heterocyclic Compound is used to inhibit a parasite or parasitic activity. In certain embodiments, treatment or prevention of cryptosporidiosis can be affected by administering a Heterocyclic Compound, either alone or in combination with another active agent as part of a combination therapy. In certain embodiments, the inhibition of a parasite or parasitic activity can be affected by administering a Heterocyclic Compound, either alone or in combination with another active agent as part of a combination therapy. The term “combination” as in the phrase “in combination with another active agent” includes co-administration of a first agent and a second agent, which for example may be dissolved or intermixed in the same pharmaceutically acceptable carrier, or administration of a first agent, followed by the second agent, or administration of the second agent, followed by the first agent. The present methods and compositions, therefore, include methods of combination therapeutic treatment and combination pharmaceutical compositions. The term “combination therapy” refers to the administration of two or more therapeutic substances, such as a compound described herein and another drug (e.g., an anti-parasitic agent such as nitazoxanide and azithromycin, an anti-motility agent such as loperamide and its derivatives). The other drug(s) may be administered concomitant with, prior to, or following the administration of a Heterocyclic Compound.

In one embodiment, provided is a method for the treatment or prevention of cryptosporidiosis, the methods comprising administering to a subject an effective amount of a Heterocyclic Compound in combination with one or more anti-parasitic agent. In one embodiment, the treatment of cryptosporidiosis comprises administration of one or more anti-parasitic agents such as nitazoxanide and azithromycin. In one embodiment, the treatment of cryptosporidiosis comprises administration of one or more an anti-motility agent such as loperamide and its derivatives.

In one embodiment, provided is a method for the inhibition of a parasite or parasitic activity, the methods comprising administering to a subject an effective amount of a Heterocyclic Compound in combination with one or more anti-parasitic agent. In one embodiment, the inhibition of a parasite or parasitic activity comprises administration of one or more anti-parasitic agents such as nitazoxanide and azithromycin. In one embodiment, the treatment of cryptosporidiosis comprises administration of one or more an anti-motility agent such as loperamide and its derivatives.

### PHARMACEUTICAL COMPOSITIONS AND ROUTES OF ADMINISTRATION

Provided herein are pharmaceutical compositions comprising an effective amount of a Heterocyclic Compound, as described herein, and a pharmaceutically acceptable carrier, excipient or vehicle.

The Heterocyclic Compounds can be administered to a subject enterally (for example, orally, rectally), topically, or parenterally (for example, intravenously, intramuscularly, subcutaneously), in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrrolidone or aluminum stearate), a dispersing agent (e.g., hydroxypropylmethylcellulose), a diluent (e.g., water), a cosolvent (e.g., propylene glocyl/glycofurol), a buffer, a copolymer (e.g., poly(lactic-co-glycolic acid, i.e. PLGA), and base wax (e.g., cocoa butter, white petrolatum or polyethylene glycol). The effective amount of the Heterocyclic Compound in the pharmaceutical composition may be at a level that will exercise the desired effect; for example, about 0.005 mg/kg of a subject's body weight to about 20 mg/kg of a subject's body weight in unit dosage for both oral and parenteral administration.

The dose of a Heterocyclic Compound to be administered to a subject is rather widely variable and can be subject to the judgment of a health-care practitioner or veterinarian. In general, the Heterocyclic Compound can be administered one to four times a day in a dose of about 0.5 mg/kg of a subject's body weight to about 20 mg/kg of a subject's body weight in a subject, but the above dosage may be properly varied depending on the age, body weight and medical condition of the subject and the type of administration. In one embodiment, the dose is about 0.1 mg/kg of a subject's body weight to about 3 mg/kg of a subject's body weight, about 0.5 mg/kg of a subject's body weight to about 2 mg/kg of a subject's body weight, about 1 mg/kg of a subject's body weight to about 2 mg/kg of a subject's body weight or about 1.5 mg/kg of a subject's body weight to about 2 mg/kg of a subject's body weight. In one embodiment, the dose is about 1 mg/kg of a subject's body weight to about 3 mg/kg of a subject's body weight. In one embodiment, the dose is about 0.5 mg/kg of a subject's body weight to about 1 mg/kg of a subject's body weight. In one embodiment, the dose is about 1 mg/kg of a subject's body weight to about 2 mg/kg of a subject's body weight. In one embodiment, the dose is about 1.5, 1.6, 1.7, 1.8, 1.9, 2.0, 2.1, 2.2, 2.3, 2.4, 2.5, 2.6, 2.7, 2.8, 2.9, 3.0 mg/kg of a subject's body weight. In one embodiment, one dose is given per day. In any given case, the amount of the Heterocyclic Compound administered will depend on such factors as the solubility of the active component, the formulation used and the route of administration. In one embodiment, application of a topical concentration provides intracellular exposures or concentrations of about 0.01-10 μM.

In another embodiment, provided herein are methods for the treatment or prevention of a disease or disorder and/or inhibition of a parasite or parasitic activity comprising the administration of about 1 mg/day to about 1200 mg/day of a Heterocyclic Compound to a subject affected by parasitic infections. In another embodiment, provided herein are methods for the treatment or prevention of a disease or disorder and/or inhibition of a parasite or parasitic activity comprising the administration of about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of a Heterocyclic Compound to a subject affected by parasitic infections. In one embodiment, the methods for the treatment of a disease or disorder and/or inhibition of a parasite or parasitic activity comprise the administration of about 0.375 mg/day to about 750 mg/day of a Heterocyclic Compound to a subject affected by parasitic infections. In one embodiment, the methods for the treatment of a disease or disorder and/or inhibition of a parasite or parasitic activity comprise the administration of about 0.75 mg/day to about 375 mg/day of a Heterocyclic Compound to a subject affected by parasitic infections. In one embodiment, the methods for the treatment of a disease or disorder and/or inhibition of a parasite or parasitic activity comprise the administration of about 3.75 mg/day to about 75 mg/day of a Heterocyclic Compound to a subject affected by parasitic infections. In one embodiment, the methods for the treatment of a disease or disorder and/or inhibition of a parasite or parasitic activity comprise the administration of about 7.5 mg/day to about 55 mg/day of a Heterocyclic Compound to a subject affected by parasitic infections. In one embodiment, the methods for the treatment of a disease or disorder and/or inhibition of a parasite or parasitic activity comprise the administration of about 18 mg/day to about 37 mg/day of a Heterocyclic Compound to a subject affected by parasitic infections.

In another embodiment, provided herein are unit dosage formulations that comprise between about 1 mg and 200 mg, about 35 mg and about 1400 mg, about 125 mg and about 1000 mg, about 250 mg and about 1000 mg, or about 500 mg and about 1000 mg of a Heterocyclic Compound. In one embodiment, the unit dosage formulations comprise between about 1 mg and 200 mg of a Heterocyclic Compound. In one embodiment, the unit dosage formulations comprise between about 35 mg and about 1400 mg of a Heterocyclic Compound. In one embodiment, the unit dosage formulations comprise between about 125 mg and about 1000 mg of a Heterocyclic Compound. In one embodiment, the unit dosage formulations comprise between about 250 mg and about 1000 mg of a Heterocyclic Compound. In one embodiment, the unit dosage formulations comprise between about 500 mg and about 1000 mg of a Heterocyclic Compound.

In a particular embodiment, provided herein are unit dosage formulations comprising about 100 mg or 400 mg of a Heterocyclic Compound.

In another embodiment, provided herein are unit dosage formulations that comprise 1 mg, 5 mg, 10 mg, 15 mg, 20 mg, 30 mg, 35 mg, 40 mg, 50 mg, 70 mg, 100 mg, 125 mg, 130, mg, 140 mg, 175 mg, 200 mg, 250 mg, 280 mg, 350 mg, 500 mg, 560 mg, 700 mg, 750 mg, 1000 mg or 1400 mg of a Heterocyclic Compound. In one embodiment, the unit dosage formulations comprise 1 mg of a Heterocyclic Compound. In one embodiment, the unit dosage formulations comprise 5 mg of a Heterocyclic Compound. In one embodiment, the unit dosage formulations comprise 10 mg of a Heterocyclic Compound. In one embodiment, the unit dosage formulations comprise 15 mg of a Heterocyclic Compound. In one embodiment, the unit dosage formulations comprise 20 mg of a Heterocyclic Compound. In one embodiment, the unit dosage formulations comprise 25 mg of a Heterocyclic Compound. In one embodiment, the unit dosage formulations comprise 30 mg of a Heterocyclic Compound. In one embodiment, the unit dosage formulations comprise 35 mg of a Heterocyclic Compound. In one embodiment, the unit dosage formulations comprise 40 mg of a Heterocyclic Compound. In one embodiment, the unit dosage formulations comprise 50 mg of a Heterocyclic Compound. In one embodiment, the unit dosage formulations comprise 70 mg of a Heterocyclic Compound. In one embodiment, the unit dosage formulations comprise 100 mg of a Heterocyclic Compound. In one embodiment, the unit dosage formulations comprise 125 mg of a Heterocyclic Compound. In one embodiment, the unit dosage formulations comprise 130 mg of a Heterocyclic Compound. In one embodiment, the unit dosage formulations comprise 140 mg of a Heterocyclic Compound. In one embodiment the unit dosage formulations comprise 175 mg of a Heterocyclic Compound. In one embodiment, the unit dosage formulations comprise 200 mg of a Heterocyclic Compound. In one embodiment, the unit dosage formulations comprise 250 mg of a Heterocyclic Compound. In one embodiment, the unit dosage formulations comprise 280 mg of a Heterocyclic Compound. In one embodiment, the unit dosage formulations comprise 350 mg of a Heterocyclic Compound. In one embodiment, the unit dosage formulations comprise 500 mg of a Heterocyclic Compound. In one embodiment, the unit dosage formulations comprise 560 mg of a Heterocyclic Compound. In one embodiment, the unit dosage formulations comprise 700 mg of a Heterocyclic Compound. In one embodiment, the unit dosage formulations comprise 750 mg of a Heterocyclic Compound. In one embodiment, the unit dosage formulations comprise 1000 mg of a Heterocyclic Compound. In one embodiment, the unit dosage formulations comprise 1400 mg of a Heterocyclic Compound.

A Heterocyclic Compound can be administered once, twice, three, four or more times daily. In a particular embodiment, doses of 600 mg or less are administered as a once daily dose and doses of more than 600 mg are administered twice daily in an amount equal to one half of the total daily dose.

A Heterocyclic Compound can be administered orally for reasons of convenience. In one embodiment, when administered orally, a Heterocyclic Compound is administered with a meal and water. In another embodiment, the Heterocyclic Compound is dispersed in water or consumable liquid (e.g., apple juice, orange juice, or nutritional drink) and administered orally as a suspension.

The Heterocyclic Compound can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin, or by local ocular (i.e., subconjunctival, intravitreal, retrobulbar, intracameral). The mode of administration is left to the discretion of the health-care practitioner or veterinarian and can depend in-part upon the site of the medical condition.

In one embodiment, provided herein are capsules containing a Heterocyclic Compound without an additional carrier, excipient or vehicle.

In another embodiment, provided herein are compositions comprising an effective amount of a Heterocyclic Compound and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories, suspensions, gels, intra-ruminal devices (e.g., for prolonged prophylaxis or controlled release), implants, topical pour-ons, transdermal delivery gels, spot-ons, implants (including devices, gels, liquids (e.g., PLGA), and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a Heterocyclic Compound with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the Heterocyclic Compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a Heterocyclic Compound as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the Heterocyclic Compound can be delayed or prolonged by proper formulation. For example, a slowly soluble pellet of the Heterocyclic Compound can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the Heterocyclic Compound in oily or emulsified vehicles, or adding amounts of PLGA, that allow it to disperse slowly in the serum.

### Examples

The following Examples are presented by way of illustration, not limitation. Compounds are named using the automatic name generating tool provided in Chemdraw Ultra 17.0 (Cambridgesoft), which generates systematic names for chemical structures, with support for the Cahn-Ingold-Prelog rules for stereochemistry. One skilled in the art can modify the procedures set forth in the illustrative examples to arrive at the desired products.

Abbreviations used:

| | |
|---|---|
| DCM | Dichloromethane |
| DMF | N,N-Dimethylformamide |
| DMSO | Dimethylsulfoxide |
| ESI | Electrospray ionization |
| EtOAc | Ethyl acetate |
| H2O | Water |
| HPLC | High performance liquid chromatography |
| LCMS | Liquid chromatography mass spectrometry |
| MeCN or ACN | Acetonitrile |
| MeOH | Methanol |
| MS | Mass spectrometry |
| NMR | Nuclear magnetic resonance |
| Pd/C | Palladium (0) on carbon |
| THF | Tetrahydrofuran |
| TLC | Thin layer chromatography |
| Xantphos | 4,5-Bis(diphenylphosphino)-9,9-dimethylxanthene |

### COMPOUND SYNTHESIS

### Example 1: 2-(2-Methylpyridin-4-yl)-N-(tetrahydro-2H-pyran-4-yl)-1H-pyrrolo[3,2-c]pyridin-6-amine

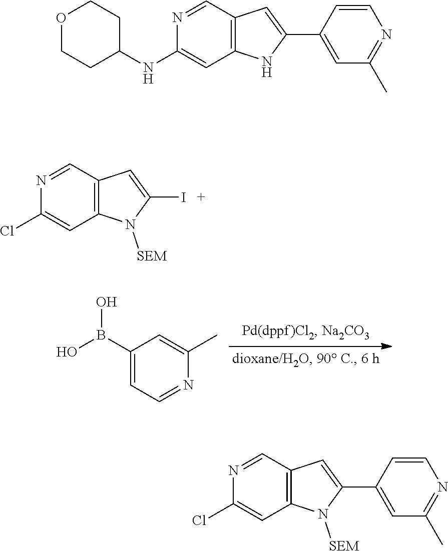

6-Chloro-2-(2-methylpyridin-4-yl)-1-((2-(trimethylsilyl)ethoxy)methyl)-1H-pyrrolo[3,2-c]pyridine. To a solution of 6-chloro-2-iodo-1-((2-(trimethylsilyl)ethoxy)methyl)-1H-pyrrolo[3,2-c]pyridine (1.5 g, 3.67 mmol) and (2-methyl-4-pyridyl)boronic acid (653.33 mg, 4.77 mmol) in dioxane (20 mL) were added a solution of sodium carbonate (777.93 mg, 7.34 mmol) in water (2 mL) followed by [1,1′-bis(diphenylphosphino)ferrocene]dichloropalladium(ii) (134.26 mg, 018 mmol) under nitrogen. The mixture was stirred at 90° C. for 3 h under nitrogen. LCMS indicated 6-chloro-2-iodo-1-((2-(trimethylsilyl)ethoxy)methyl)-1H-pyrrolo[3,2-c]pyridine remained (32%) and a peak (37%) with desired mass was detected. Then (2-methyl-4-pyridyl)boronic acid (301.54 mg, 2.20 mmol), [1,1′-bis (diphenylphosphino)ferrocene]dichloropalladium(ii) (67.13 mg, 0.92 mmol) and sodium carbonate (388.96 mg, 3.67 mmol) were added to the mixture, the mixture was stirred at 90° C. for 3 h under nitrogen. LCMS indicated 6-chloro-2-iodo-1-((2-(trimethylsilyl)ethoxy)methyl)-1H-pyrrolo[3,2-c]pyridine was consumed and a peak (42%) with desired mass was detected. The mixture was filtered with a pad of Celite, the filtrate was concentrated under vacuum to give a residue. The residue was purified by flash silica gel chromatography (ISCO®; 20 g SepaFlash® Silica Flash Column, Eluent of 0˜30% Ethyl acetate/Petroleum ether gradient @ 35 mL/min) to give 6-chloro-2-(2-methylpyridin-4-yl)-1-((2-(trimethylsilyl)ethoxy)methyl)-1H-pyrrolo[3,2-c]pyridine (1.05 g, 2.81 mmol, 76.51% yield) as a yellow oil. ¹H NMR (400 MHz, DMSO-d₆) δ=8.72 (d, J=0.6 Hz, 1H), 8.57 (d, J=5.0 Hz, 1H), 7.92 (s, 1H), 7.58 (s, 1H), 7.53 (dd, J=1.2, 5.1 Hz, 1H), 7.04 (d, J=0.6 Hz, 1H), 5.62 (s, 2H), 3.48-3.44 (m, 2H), 2.55 (s, 3H), 0.80-0.76 (m, 2H), −0.12-0.14 (m, 9H).

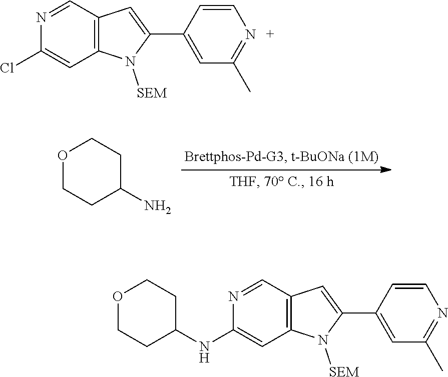

2-(2-Methylpyridin-4-yl)-N-(tetrahydro-2H-pyran-4-yl)-1-((2-(trimethylsilyl)ethoxy)methyl)-1H-pyrrolo[3,2-c]pyridin-6-amine. To a solution of 6-chloro-2-(2-methylpyridin-4-yl)-1-((2-(trimethylsilyl)ethoxy)methyl)-1H-pyrrolo[3,2-c]pyridine (900 mg, 2.41 mmol) and tetrahydro-2H-pyran-4-amine (730.30 mg, 7.22 mmol) in THF (15 mL) was added Sodium tert-Butoxide (1 M, 4.81 mL) and Methanesulfonato(2-dicyclohexylphosphino-3,6-dimethoxy-2,4,6-tri-i-propyl-1,1-biphenyl)(2-amino-1,1-biphenyl-2-yl)palladium(II) (218.17 mg, 0.24 mmoL) under nitrogen, the mixture was stirred at 70° C. for 16 h under nitrogen atmosphere. LCMS indicated 6-chloro-2-(2-methylpyridin-4-yl)-1-((2-(trimethylsilyl) ethoxy)methyl)-1H-pyrrolo[3,2-c]pyridine was consumed and a peak (47%) with desired mass was detected. The mixture was filtered with a pad of Celite, the filtrate was concentrated under vacuum to give a residue. The residue was purified by column chromatography (100-200 mesh silica gel, Petroleum ether/Ethyl acetate=5/1, 0/1). The eluent was concentrated under vacuum to give a crude product. To the crude product in methanol (1 mL) and dichloromethane (10 mL) was added SiliaMetS Thiol (0.5 g), the suspension was stirred for 24 h, then filtered with a pad of Celite. The filtrated was concentrated under vacuum to give 2-(2-methylpyridin-4-yl)-N-(tetrahydro-2H-pyran-4-yl)-1-((2-(trimethylsilyl)ethoxy)methyl)-1H-pyrrolo[3,2-c]pyridin-6-amine (960 mg, 2.19 mmol, 90.94% yield) as a yellow oil. ¹H NMR (400 MHz, DMSO-d₆) δ 8.50 (d, J=5.1 Hz, 1H), 8.36 (d, J=0.6 Hz, 1H), 7.51 (s, 1H), 7.45 (dd, J=1.3, 5.1 Hz, 1H), 6.78 (s, 1H), 6.56 (s, 1H), 6.07 (d, J=8.1 Hz, 1H), 5.41 (s, 2H), 3.89-3.86 (m, 2H), 3.51-3.42 (m, 4H), 1.92-1.88 (m, 2H), 1.48-1.45 (m, 2H), 0.85-0.81 (m, 2H), −0.05-0.12 (m, 9H).

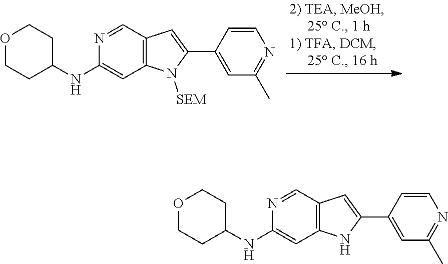 [SMILES: Cc1cc(-c2cc3cnc(NC4CCOCC4)cc3n2COCC[Si](C)(C)C)ccn1 ; Cc1cc(-c2cc3cnc(NC4CCOCC4)cc3[nH]2)ccn1]

2-(2-Methylpyridin-4-yl)-N-(tetrahydro-2H-pyran-4-yl)-1H-pyrrolo[3,2-c]pyridin-6-amine. To a solution of 2-(2-methylpyridin-4-yl)-N-(tetrahydro-2H-pyran-4-yl)-1-((2-(trimethylsilyl)ethoxy)methyl)-1H-pyrrolo[3,2-c]pyridin-6-amine (960 mg, 2.19 mmol) in dichloromethane (10 mL) was added trifluoroacetic acid (15.40 g, 135.06 mmol, 10 mL), the mixture was stirred at 25° C. for 16 h. LCMS indicated 2-(2-methylpyridin-4-yl)-N-(tetrahydro-2H-pyran-4-yl)-1-((2-(trimethylsilyl)ethoxy)methyl)-1H-pyrrolo[3,2-c]pyridin-6-amine remained (4%) and a peak (28%) with desired mass. The mixture was concentrated under vacuum to give a residue. To the residue in methanol (10 mL) was added triethylamine (7.27 g, 71.85 mmol, 10 mL), the mixture was stirred at 25° C. for 2 h. LCMS indicated 2-(2-methylpyridin-4-yl)-N-(tetrahydro-2H-pyran-4-yl)-1-((2-(trimethylsilyl)ethoxy)methyl)-1H-pyrrolo[3,2-c]pyridin-6-amine was consumed and a peak(76%) with desired mass was detected. The mixture was concentrated under reduced pressure to give a residue. The residue was purified by prep-HPLC (column: Phenomenex luna C18 150*40 mm*15 um; mobile phase: [water (0.225% FA)-ACN];B %: 1%-17%,10 min) then dried by lyophilization to give 2-(2-methylpyridin-4-yl)-N-(tetrahydro-2H-pyran-4-yl)-1H-pyrrolo[3,2-c]pyridin-6-amine (228.37 mg, 0.74 mmol, 33.80% yield, 99.9% purity) as a yellow solid. ¹H NMR (400 MHz, DMSO-d₆) δ 12.59 (s, 1H), 8.69 (d, J=5.6 Hz, 1H), 8.64 (s, 1H), 8.14 (s, 0.23H), 7.99 (s, 1H), 7.91 (d, J=5.6 Hz, 1H), 7.77 (s, 1H), 7.48 (s, 1H), 6.80 (s, 1H), 3.93-3.90 (m, 2H), 3.83-3.82 (m, 1H), 3.48-3.42 (m, 2H), 2.63 (s, 3H), 1.96-1.93 (m, 2H), 1.53-1.50 (m, 2H). MS (ESI): m/z 309.1 [M+1]⁺.

### Example 2: 2-(6-Methylpyrimidin-4-yl)-N-(tetrahydro-2H-pyran-4-yl)-1H-pyrrolo[3,2-c]pyridin-6-amine

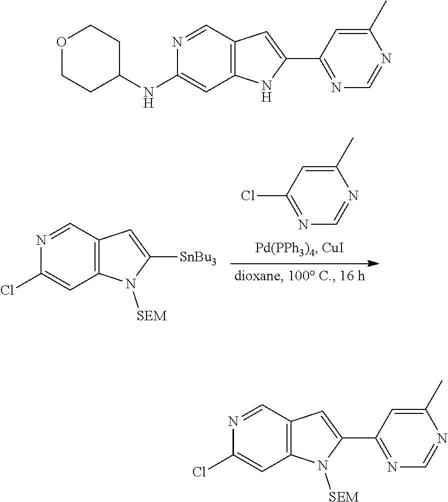

6-Chloro-2-(6-methylpyrimidin-4-yl)-1-((2-(trimethylsilyl)ethoxy)methyl)-1h-pyrrolo[3,2-c]pyridine. To a solution of 4-chloro-6-methylpyrimidine (340 mg, 2.64 mmol) and 6-chloro-2-(tributylstannyl)-1-((2-(trimethylsilyl)ethoxy)methyl)-1H-pyrrolo[3,2-c]pyridine (1 g, 1.75 mmol) in 1,4-Dioxane (10 mL) was added cuprous iodide (66.6 mg, 0.3500 mmol) and tetrakis(triphenylphosphine)palladium(0) (202.06 mg, 0.1700 mmol). The reaction mixture was stirred at 100° C. for 16 h under nitrogen. LCMS showed 6-chloro-2-(tributylstannyl)-1-((2-(trimethylsilyl)ethoxy)methyl)-1H-pyrrolo[3,2-c]pyridine was consumed completely and a peak (10.10%) with desired mass. The mixture was cooled and concentrated in reduced pressure. The residue was poured into water (40 mL). The aqueous phase was extracted with ethyl acetate (40 mL×3). The combined organic phase was washed with brine (40 mL×2), dried over anhydrous sodium sulfate, filtered and concentrated under vacuum. The residue was purified by silica gel chromatography (200-300 mesh silica gel, Petroleum ether/Ethyl acetate=100/1, 1/1, Rf=0.6, Petroleum ether/Ethyl acetate=1/1), to give 6-chloro-2-(6-methylpyrimidin-4-yl)-1-((2-(trimethylsilyl)ethoxy)methyl)-1H-pyrrolo[3,2-c]pyridine (300 mg, 0.6017 mmol, 34.41% yield) as a yellow solid. LCMS (ESI): m/z 375.1 [M+1]⁺.

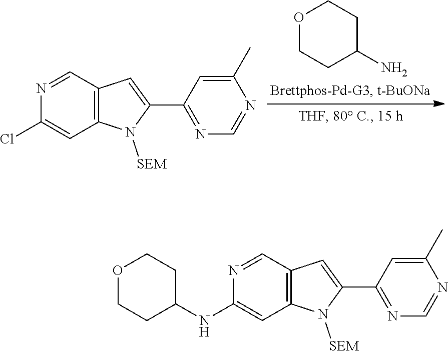 [SMILES: Cc1cc(-c2cc3cnc(Cl)cc3n2COCC[Si](C)(C)C)ncn1 ; NC1CCOCC1 ; Cc1cc(-c2cc3cnc(NC4CCOCC4)cc3n2COCC[Si](C)(C)C)ncn1]

2-(6-methylpyrimidin-4-yl)-N-(tetrahydro-2H-pyran-4-yl)-1-((2-(trimethylsilyl)ethoxy)methyl)-1H-pyrrolo[3,2-c]pyridin-6-amine. To a solution of tetrahydro-2H-pyran-4-amine (242.8 mg, 2.4 mmol) and 6-chloro-2-(6-methylpyrimidin-4-yl)-1-((2-(trimethylsilyl)ethoxy)methyl)-1H-pyrrolo[3,2-c]pyridine (300. mg, 0.8000 mmol) in THF (10 mL) was added methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (145.06 mg, 0.1600 mmol) and sodium tert-butoxide (1.6 mL, 1.6 mmol, 1 M in THF), the reaction mixture was stirred at 80° C. for 15 h under nitrogen. LCMS showed 6-chloro-2-(6-methylpyrimidin-4-yl)-1-((2-(trimethylsilyl)ethoxy)methyl)-1H-pyrrolo[3,2-c]pyridine was consumed completely and a peak (27.8%) with desired mass. The mixture was cooled and concentrated in reduced pressure. The residue was poured into water (40 mL). The aqueous phase was extracted with ethyl acetate (40 mL×3). The combined organic phase was washed with brine (30 mL×2), dried over anhydrous sodium sulfate, filtered and concentrated under vacuum. The residue was purified by silica gel chromatography (200-300 mesh silica gel, Petroleum ether/Ethyl acetate=100/1, 1/1), rf=0.4, Petroleum ether/Ethyl acetate=1/1) to give 2-(6-methylpyrimidin-4-yl)-N-(tetrahydro-2H-pyran-4-yl)-1-((2-(trimethylsilyl)ethoxy)methyl)-1H-pyrrolo[3,2-c]pyridin-6-amine (280 mg, 0.5153 mmol, 64.396% yield) as a yellow solid. LCMS (ESI): m/z 440.2 [M+1]⁺.

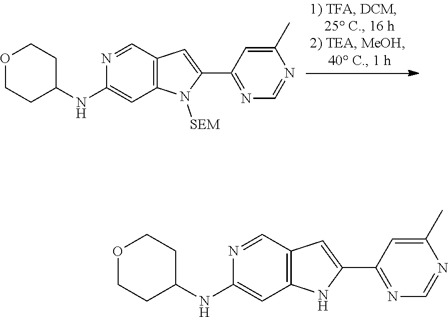 [SMILES: Cc1cc(-c2cc3cnc(NC4CCOCC4)cc3n2COCC[Si](C)(C)C)ncn1 ; Cc1cc(-c2cc3cnc(NC4CCOCC4)cc3[nH]2)ncn1]

2-(6-Methylpyrimidin-4-yl)-N-tetrahydropyran-4-yl-1H-pyrrolo[3,2-c]pyridin-6-amine. To a solution of 2-(6-methylpyrimidin-4-yl)-N-(tetrahydro-2H-pyran-4-yl)-1-((2-(trimethylsilyl)ethoxy)methyl)-1H-pyrrolo[3,2-c]pyridin-6-amine (280. mg, 0.6400 mmol) in dichloromethane (5 mL) was added trifluoroacetic acid (5. mL, 0.6400 mmol), the reaction mixture was stirred at 25° C. for 16 h. LCMS showed 2-(6-methylpyrimidin-4-yl)-N-(tetrahydro-2H-pyran-4-yl)-1-((2-(trimethylsilyl)ethoxy)methyl)-1H-pyrrolo[3,2-c]pyridin-6-amine was consumed completely and a peak (70%) with mass of (2-(6-methylpyrimidin-4-yl)-6-((tetrahydro-2H-pyran-4-yl)amino)-1H-pyrrolo[3,2-c]pyridin-1-yl)methanol. The mixture was concentrated in reduced pressure. To the residue in methanol (5 mL) was added triethylamine (5 mL, 35.87 mmol), the reaction mixture was stirred at 40° C. for another 1 h. LCMS showed (2-(6-methylpyrimidin-4-yl)-6-((tetrahydro-2H-pyran-4-yl)amino)-1H-pyrrolo[3,2-c]pyridin-1-yl)methanol was consumed completely and a peak (74.5%) with desired mass. The mixture was concentrated in reduced pressure to give a residue. The residue was purified by prep-HPLC (column Phenomenex luna C18 150*25 mm*10 um; mobile phase: [water (0.225% FA)-ACN]; B %: 4%-34%, 9 min) followed by lyophilization to give 2-(6-methylpyrimidin-4-yl)-N-(tetrahydro-2H-pyran-4-yl)-1H-pyrrolo[3,2-c]pyridin-6-amine (26.61 mg, 0.0833 mmol, 13.073% yield, formic acid) as a yellow solid. ¹H NMR (400 MHz, DMSO-d₆) δ 11.44 (s, 1H), 8.99 (s, 1H), 8.43 (s, 1H), 8.18 (s, 1H), 7.84 (s, 1H), 7.29 (s, 1H), 6.40 (s, 1H), 6.10 (s, 1H), 3.89-3.87 (m, 3H), 3.46-3.39 (m, 2H), 2.49 (s, 3H), 1.90-1.83 (m, 2H), 1.50-1.37 (m, 2H). LCMS (ESI): m/z 310.2 [M+1]⁺.

### Example 3: 1-Methyl-2-(6-methylpyrimidin-4-yl)-N-(tetrahydro-2H-pyran-4-yl)-1H-pyrrolo[3,2-c]pyridin-6-amine

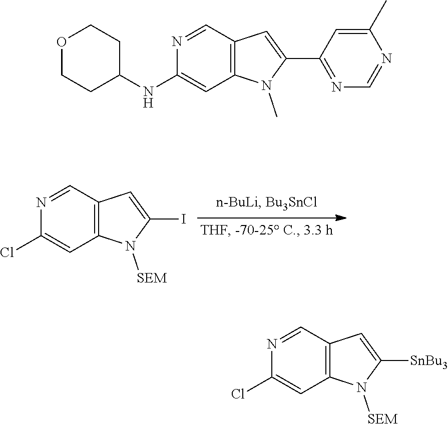

6-Chloro-2-(tributylstannyl)-1-((2-(trimethylsilyl)ethoxy)methyl)-1H-pyrrolo[3,2-c]pyridine. To a mixture of 6-chloro-2-iodo-1-((2-(trimethylsilyl)ethoxy)methyl)-1H-pyrrolo[3,2-c]pyridine (20. g, 48.93 mmol) in THF (300 mL) was added Butyllithium (29.36 mL, 73.4 mmol, 2.5 M) at −70° C. over 10 min, the mixture was stirred at this temperature for 10 min, and then tributyl(chloro)stannane (17.13 mL, 68.5 mmol) was added drop wise at -70° C. The resulting mixture was stirred at 25° C. for 3 h. TLC (Petroleum ether:Ethyl acetate=5:1) indicated starting material was consumed and one major new spot with lower polarity. Rf SM=0.4, Rf DP=0.6. The mixture was poured into water (500 mL). The aqueous phase was extracted with ethyl acetate (300 mL×3). The combined organic phase was washed with brine (150 mL×2), dried over anhydrous sodium sulfate, filtered and concentrated under vacuum. The residue was purified by silica gel chromatography (100-200 mesh silica gel, Petroleum ether/Ethyl acetate=1/0, 15/1) to give 6-chloro-2-(tributylstannyl)-1-((2-(trimethylsilyl)ethoxy)methyl)-1H-pyrrolo[3,2-c]pyridine (23.7 g, 41.44 mmol, 84.69% yield) as a yellow oil. LCMS (ESI): m/z 573.0 [M+1]⁺. ¹H NMR (400 MHz, DMSO-d₆) δ 8.57 (s, 1H), 7.78 (s, 1H), 6.76 (s, 1H), 5.49 (s, 2H), 3.42-3.38 (m, 2H), 1.62-1.51 (m, 6H), 1.31-1.29 (m, 6H), 1.17-1.15 (m, 6H), 0.89-0.82 (m, 9H), -0.08-0.09 (m, 9H).

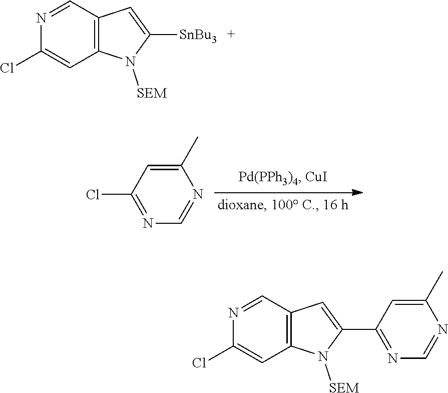

6-Chloro-2-(6-methylpyrimidin-4-yl)-1-((2-(trimethylsilyl)ethoxy)methyl)-1H-pyrrolo[3,2-c]pyridine. To a mixture of 6-chloro-2-(tributylstannyl)-1-((2-(trimethylsilyl)ethoxy)methyl)-1H-pyrrolo[3,2-c]pyridine (20. g, 34.97 mmol) in 1,4-Dioxane (200 mL) was added 4-chloro-6-methylpyrimidine (4.5 g, 34.97 mmol), cuprous iodide (0.67 g, 3.5 mmol) and tetrakis[triphenylphosphine]palladium(0) (4.04 g, 3.5 mmol). The mixture was stirred at 100° C. for 16 h under nitrogen atmosphere. LCMS showed 6-chloro-2-(tributylstannyl)-1-((2-(trimethylsilyl)ethoxy)methyl)-1H-pyrrolo[3,2-c]pyridine was consumed completely and a peak (62%) with desired mass. The mixture was concentrated under reduced pressure to give a residue. The residue was purified by flash silica gel chromatography (Biotage, 220 g SepaFlash® Silica Flash Column, Eluent of 10˜60% Ethyl acetate/Petroleum ether gradient @ 80 mL/min) to give 6-chloro-2-(6-methylpyrimidin-4-yl)-1-((2-(trimethylsilyl)ethoxy)methyl)-1H-pyrrolo[3,2-c]pyridine (11.4 g, 30.41 mmol, 86.94% yield) as a yellow oil. LCMS (ESI): m/z 375.1 [M+1]⁺. ¹H NMR (400 MHz, DMSO-d₆) δ 9.13 (s, 1H), 8.79 (s, 1H), 7.98 (s, 1H), 7.90 (s, 1H), 7.51 (s, 1H), 6.18 (s, 2H), 3.33-3.31 (m, 2H), 2.54 (s, 3H), 0.69-0.65 (m, 2H), −0.24-0.25 (m, 9H).

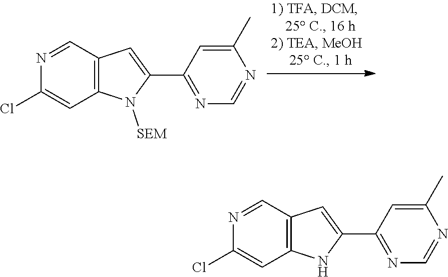 [SMILES: Cc1cc(-c2cc3cnc(Cl)cc3n2COCC[Si](C)(C)C)ncn1 ; Cc1cc(-c2cc3cnc(Cl)cc3[nH]2)ncn1]

6-Chloro-2-(6-methylpyrimidin-4-yl)-1H-pyrrolo[3,2-c]pyridine. To a mixture of 2-[[6-chloro-2-(6-methylpyrimidin-4-yl)pyrrolo[3,2-c]pyridin-1-yl]methoxy]ethyl-trimethyl-silane (11.4 g, 30.4 mmol) in dichloromethane (100 mL) was added Trifluoroacetic acid (100. mL, 30.4 mmol). The mixture was stirred at 25° C. for 16 h. LCMS showed 2-[[6-chloro-2-(6-methylpyrimidin-4-yl) pyrrolo[3,2-c]pyridin-1-yl]methoxy]ethyl-trimethyl-silane was consumed completely and a peak (73%) with desired mass. The mixture was concentrated under reduced pressure to give a residue. To the residue in Methanol (100 mL) was added triethylamine (100. mL, 30.4 mmol), the mixture was stirred at 25° C. for 1 h. The mixture was concentrated under reduced pressure to give a residue. TLC (Petroleum ether:Ethyl acetate=0:1) indicated starting material was consumed and one major new spot with larger polarity and lower polarity. The mixture was poured into ammonium chloride (100 mL). The aqueous phase was extracted with ethyl acetate (50 mL). The solid precipitated from ethyl acetate/water. The solid was collected and triturated with ethyl acetate (30 mL), the suspension was filtered and the filter cake was dried under vacuum. The filter cake was triturated with methanol (30 mL) to give 6-chloro-2-(6-methylpyrimidin-4-yl)-1H-pyrrolo[3,2-c]pyridine (6.5 g, 26.57 mmol, 87.37% yield) as a yellows solid. LCMS (ESI): m/z 245.0 [M+1]⁺. ¹H NMR (400 MHz, DMSO-d₆) δ 9.08 (s, 1H), 8.73 (s, 1H), 8.08 (s, 1H), 7.53 (s, 1H), 7.45 (s, 1H), 2.52 (s, 3H).

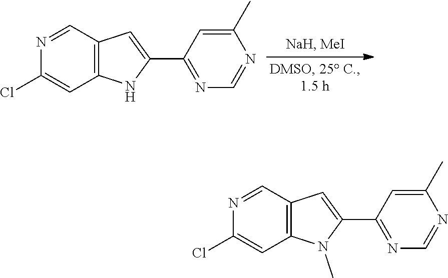 [SMILES: Cc1cc(-c2cc3cnc(Cl)cc3[nH]2)ncn1 ; Cc1cc(-c2cc3cnc(Cl)cc3n2C)ncn1]

6-Chloro-1-methyl-2-(6-methylpyrimidin-4-yl)-1H-pyrrolo[3,2-c]pyridine. To a solution of 6-chloro-2-(6-methylpyrimidin-4-yl)-1H-pyrrolo[3,2-c]pyridine (2. g, 8.17 mmol) in dimethyl sulfoxide (60 mL) was added Sodium Hydride (980.87 mg, 24.52 mmol, 60% purity) at 25° C. and then the mixture was stirred at 25° C. for 0.5 h, then methyl iodide (0.31 mL, 4.9 mmol) was added in the mixture at 25° C., the mixture was stirred at 25° C. for 1 h. LCMS showed 6-chloro-2-(6-methylpyrimidin-4-yl)-1H-pyrrolo[3,2-c]pyridine was consumed completely and a peak (71%) with desired mass. The residue was poured into ammonium chloride (200 mL). The aqueous phase was extracted with ethyl acetate (100 mL×3). The combined organic phase was washed with brine (50 mL×2), dried over anhydrous sodium sulfate, filtered and concentrated under vacuum. The residue was purified by prep-HPLC (column: Unisil 3-100 C18 Ultra 150*50 mm*3 um; mobile phase: [water(0.225% FA)-ACN]; B %: 19%-49%, 10 min) followed by lyophilization to give 6-chloro-1-methyl-2-(6-methylpyrimidin-4-yl)-1H-pyrrolo[3,2-c]pyridine (800 mg, 3.09 mmol, 37.83% yield) as yellow solid. LCMS (ESI): m/z 259.0 [M+1]⁺.

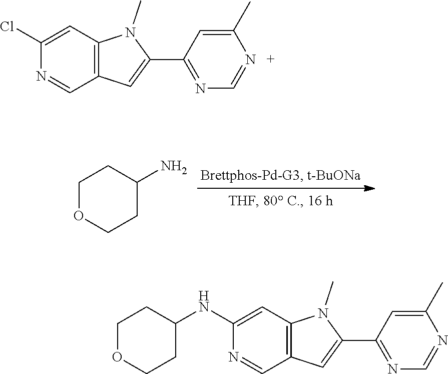

1-Methyl-2-(6-methylpyrimidin-4-yl)-N-(tetrahydro-2H-pyran-4-yl)-1H-pyrrolo[3,2-c]pyridin-6-amine. To a mixture of 6-chloro-1-methyl-2-(6-methylpyrimidin-4-yl)-1H-pyrrolo[3,2-c]pyridine (150. mg, 0.58 mmol) and tetrahydro-2H-pyran-4-amine (175.94 mg, 1.74 mmol) in tetrahydrofuran (1 mL) was added Methanesulfonato (2-dicyclohexylphosphino-3,6-dimethoxy-2,4,6-tri-i-propyl-1,1-biphenyl)(2-amino-1,1-biphenyl-2-yl)palladium(II) (52.56 mg, 0.06 mmol) and Sodium tert-Butoxide (1.16 mL, 1.16 mmol, 1 M in THF). The mixture was stirred at 80° C. for 16 h under nitrogen. LCMS showed 6-chloro-1-methyl-2-(6-methylpyrimidin-4-yl)-1H-pyrrolo[3,2-c]pyridine was consumed completely and a peak (56%) with desired mass. The mixture was filtered with a pad of silica gel (200-300 mesh silica gel) and washed with acetyl acetate (50 mL), the filtrate was concentrated under vacuum to give a residue. The residue was purified by prep-HPLC (column: Unisil 3-100 C18 Ultra 150*50 mm*3 um; mobile phase: [water(0.225% FA)-ACN]; B %: 10%-30%, 10 min) followed by lyophilization to give 1-methyl-2-(6-methylpyrimidin-4-yl)-N-(tetrahydro-2H-pyran-4-yl)-1H-pyrrolo[3,2-c]pyridin-6-amine (114.97 mg, 0.36 mmol, 61.26% yield, formic acid) as a yellow solid. Additional 2.48 mg for delivery. LCMS (ESI): m/z 324.3 [M+1]⁺. ¹H NMR (400 MHz, DMSO-d₆) δ 9.03 (s, 1H), 8.43 (s, 1H), 8.16 (s, 0.5H),7.84 (s, 1H), 7.22 (s, 1H), 6.39 (s, 1H), 6.14-6.12 (m, 1H), 3.97 (s, 3H), 3.90-3.88 (m, 3H), 3.47-3.41 (m, 2H), 2.50 (s, 3H), 1.93-1.90 (m, 2H), 1.51-1.44 (m, 2H).

### Example 4: (R)-2-(2-Methylpyridin-4-yl)-N-(tetrahydro-2h-pyran-3-yl)-1H-pyrrolo[3,2-c]pyridin-6-amine

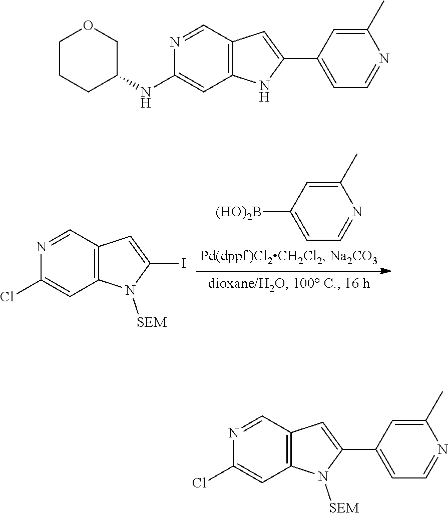

6-Chloro-2-(2-methylpyridin-4-yl)-1-((2-(trimethylsilyl)ethoxy)methyl)-1H-pyrrolo[3,2-c]pyridine. A mixture of 6-chloro-2-iodo-1-((2-(trimethylsilyl)ethoxy)methyl)-1H-pyrrolo[3,2-c]pyridine (2 g, 4.89 mmol), (2-methylpyridin-4-yl)boronic acid (670.08 mg, 4.89 mmol), 1,1′-bis(diphenylphosphino)ferrocene-palladium(ii)dichloride dichloromethane complex (399.59 mg, 0.489 mmol), sodium carbonate (1.04 g, 9.79 mmol) in 1,4-dioxane (30 mL) and water (3 mL) was degassed and purged with nitrogen for 3 times, and then the mixture was stirred at 100° C. for 16 h under nitrogen atmosphere. LCMS showed 6-chloro-2-iodo-1-((2-(trimethylsilyl)ethoxy)methyl)-1H-pyrrolo[3,2-c]pyridine was consumed completely and one main peak with desired mass. The mixture was diluted with ethyl acetate (50 mL) and filtrated, the filtrate was concentrated to afford the residue. The residue was purified by flash silica gel chromatography (ISCO®; 80 g SepaFlash® Silica Flash Column, Eluent of 0˜80% Ethyl acetate/Petroleum ether gradient @ 50 mL/min) to give 6-chloro-2-(2-methylpyridin-4-yl)-1-((2-(trimethylsilyl)ethoxy)methyl)-1H-pyrrolo[3,2-c]pyridine(1.28 g, 3.31 mmol, 67.64% yield) as a brown solid. ¹H NMR (400 MHz, CDCl₃) δ 8.72 (s, 1H), 8.64-8.62 (m, 1H), 7.45-7.44 (m, 2H), 7.40-7.38 (m, 1H), 6.78 (s, 1H), 5.41 (s, 2H), 3.59-3.55 (m, 2H), 2.66 (s, 3H), 0.96-0.92 (m, 2H), −0.01 (s, 9H).

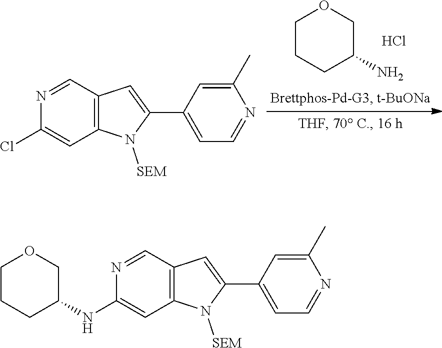

(R)-2-(2-Methylpyridin-4-yl)-N-(tetrahydro-2H-pyran-3-yl)-1-((2-(trimethylsilyl)ethoxy)methyl)-1H-pyrrolo[3,2-c]pyridin-6-amine. A mixture of 6-chloro-2-(2-methylpyridin-4-yl)-1-((2-(trimethylsilyl)ethoxy)methyl) -1H-pyrrolo[3,2-c]pyridine (400 mg, 1.07 mmol), (R)-tetrahydro-2H-pyran-3-amine hydrochloride (147.19 mg, 1.07 mmol), Methanesulfonato(2-dicyclohexylphosphino-3,6-dimethoxy-2,4,6-tri-i-propyl-1,1-biphenyl)(2-amino-1,1-biphenyl-2-yl)palladium(II) (96.96 mg, 0.107 mmol), sodium 2-methylpropan-2-olate (308.39 mg, 3.21 mmol) in THF (10 mL) was degassed and purged with nitrogen for 3 times, and then the mixture was stirred at 70° C. for 16 h under nitrogen atmosphere. LCMS showed 6-chloro-2-(2-methylpyridin-4-yl)-1-((2-(trimethylsilyl)ethoxy)methyl)-1H-pyrrolo[3,2-c]pyridine was consumed completely and one main peak with desired mass. The mixture was diluted with ethyl acetate (50 mL) and filtrated, the filtrate was concentrated to afford residue. The residue was purified by flash silica gel chromatography (ISCO®; 25 g SepaFlash® Silica Flash Column, Eluent of 0˜10% methanol/Ethyl acetate @ 25 mL/min) to give (R)-2-(2-methylpyridin-4-yl)-N-(tetrahydro-2H-pyran-3-yl)-1-((2-(trimethylsilyl)ethoxy)methyl)-1H-pyrrolo[3,2-c]pyridin-6-amine(358 mg, 0.66 mol, 72.49% yield) as a yellow gum. MS (ESI): m/z 439.1 [M+1]⁺.

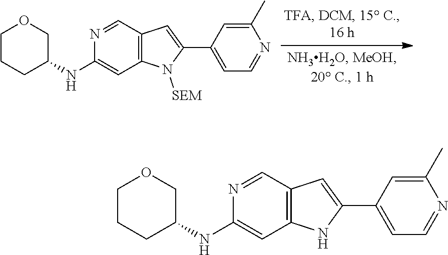 [SMILES: Cc1cc(-c2cc3cnc(N[C@@H]4CCCOC4)cc3n2COCC[Si](C)(C)C)ccn1 ; Cc1cc(-c2cc3cnc(N[C@@H]4CCCOC4)cc3[nH]2)ccn1]

(R)-2-(2-Methylpyridin-4-yl)-N-(tetrahydro-2H-pyran-3-yl)-1H-pyrrolo[3,2-c]pyridin-6-amine. A mixture of(R)-2-(2-methylpyridin-4-yl)-N-(tetrahydro-2H-pyran-3-yl)-1-((2-(trimethylsilyl)ethoxy)methyl)-1H-pyrrolo[3,2-c]pyridin-6-amine (358 mg, 0.82 mmol) in dichloromethane (2 mL) and 2,2,2-trifluoroacetic acid (2 mL) was stirred at 15° C. for 16 h. LCMS showed (R)-2-(2-methylpyridin-4-yl)-N-(tetrahydro-2H-pyran-3-yl)-1-((2-(trimethylsilyl)ethoxy)methyl)-1H-pyrrolo[3,2-c]pyridin-6-amine was consumed completely and one main peak with the mass of intermediate. To the mixture in methanol (5 mL) was added ammonium hydroxide (5 mL), stirred for 1 h then diluted with water (20 mL) and extracted with dichloromethane (20 mL×3), the combined organic layer was dried over sodium sulfate and concentrated to afford residue. The residue was purified by prep-HPLC (column: YMC-Actus Triart C18 150*30 mm*5 um; mobile phase: [water (0.05% ammonia hydroxide v/v)-ACN]; B %: 32%-52%,10 min) followed by lyophilization to give (R)-2-(2-methylpyridin-4-yl)-N-(tetrahydro-2H-pyran -3-yl)-1H-pyrrolo[3,2-c]pyridin-6-amine (122.59 mg, 0.39 mmol, 47% yield) as a yellow solid. ¹H NMR (400 MHz, DMSO-d₆) δ 11.31 (s, 1H), 8.43 (d, J=5.3 Hz, 1H), 8.35 (s, 1H), 7.61 (s, 1H), 7.53-7.52 (m, 1H), 7.05 (d, J=1.0 Hz, 1H), 6.39 (s, 1H), 5.94 (d, J=8.0 Hz, 1H), 3.93-3.90 (m, 1H), 3.82-3.76 (m, 1H), 3.75-3.72 (m, 1H), 3.36-3.30 (m, 1H), 3.11-3.07 (m, 1H), 2.50 (s, 3H), 1.97-1.95 (m, 1H), 1.77-1.66 (m, 1H), 1.64-1.42 (m, 2H). MS (ESI): m/z 309.1 [M+1]⁺.

### Example 5: N-(Tetrahydro-2H-pyran-4-yl)-2-(2-((2,2,2-trifluoroethyl)amino)pyridin-4-yl)-1H-pyrrolo[3,2-c]pyridin-6-amine

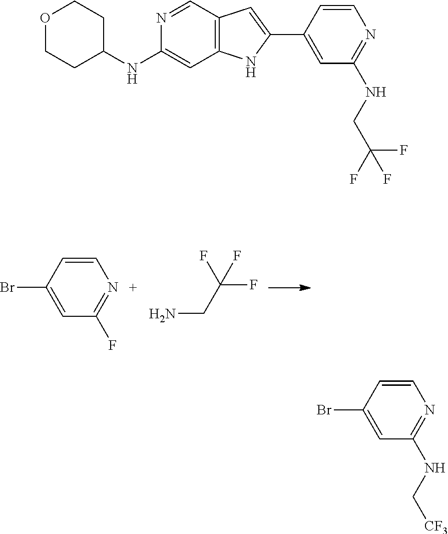

4-Bromo-N-(2,2,2-trifluoroethyl)pyridin-2-amine. To a mixture of 4-bromo-2-fluoropyridine (2000. mg, 11.36 mmol) in dimethylsulfoxide (30 mL) was added 2,2,2-trifluoroethanamine (13.04 mL, 227.29 mmol). The mixture was stirred at 150° C. for 4 h under microwave. LCMS showed 4-bromo-2-fluoropyridine was consumed completely and a peak (60%) with desired mass. The residue was poured into water (20 mL). The aqueous phase was extracted with ethyl acetate (30 mL×3). The combined organic phase was washed with brine (20 mL×2), dried over anhydrous sodium sulfate, filtered and concentrated under vacuum. The residue was purified by flash silica gel chromatography (Biotage, 24 g SepaFlash® Silica Flash Column, Eluent of 0-10% Ethyl acetate/Petroleum ether gradient @ 40 mL/min) to give 4-bromo-N-(2,2,2-trifluoroethyl) pyridin-2-amine (1000 mg, 3.921 mmol, 34.502% yield) as a white solid. H NMR (400 MHz, DMSO-d₆) δ=7.91 (d, J=5.4 Hz, 1H), 7.39-7.36 (t, J=6.5 Hz, 1H), 6.86 (d, J=1.6 Hz, 1H), 6.81 (dd, J=1.7, 5.5 Hz, 1H), 4.21-4.12 (m, 2H).

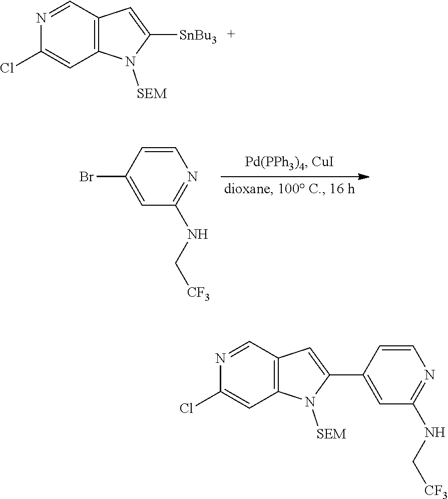

4-(6-Chloro-1-((2-(trimethylsilyl)ethoxy)methyl)-1H-pyrrolo[3,2-c]pyridin-2-yl)-N-(2,2,2-trifluoroethyl)pyridin-2-amine. To a mixture of 6-chloro-2-(tributylstannyl)-1-((2-(trimethylsilyl)ethoxy)methyl) -1H-pyrrolo[3,2-c]pyridine (1. g, 1.75 mmol) and 4-bromo-N-(2,2,2-trifluoroethyl) pyridin-2-amine (445.96 mg, 1.75 mmol) in 1,4-Dioxane (10 mL) was added cuprous iodide (33.3 mg, 0.1700 mmol) and tetrakis[triphenylphosphine]palladium(0) (202.06 mg, 0.1700 mmol). The mixture was stirred at 100° C. for 16 h under nitrogen atmosphere. LCMS showed 6-chloro-2-(tributylstannyl)-1-((2-(trimethylsilyl) ethoxy)methyl)-1H-pyrrolo[3,2-c]pyridine was consumed completely and a peak (45%) with desired mass. The mixture was concentrated under reduced pressure to give a residue. The residue was purified by flash silica gel chromatography (Biotage, 24 g SepaFlash® Silica Flash Column, Eluent of 0˜21% Ethyl acetate/Petroleum ether gradient @ 35 mL/min) to give 4-(6-chloro-1-((2-(trimethylsilyl)ethoxy)methyl)-1H-pyrrolo[3,2-c]pyridin-2-yl)-N-(2,2,2-trifluoroethyl)pyridin-2-amine (500 mg, 1.0942 mmol, 62.575% yield) as a white solid. MS (ESI): m/z 457.1 [M+1]⁺.

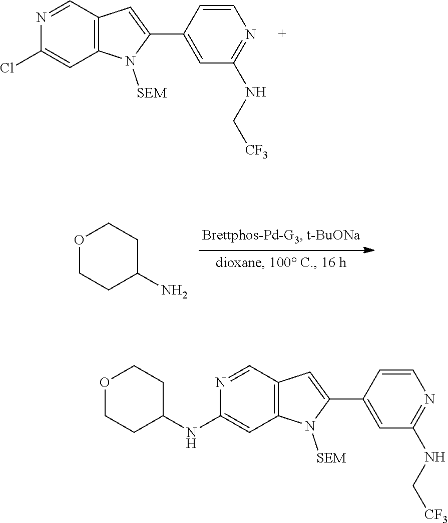

N-(Tetrahydro-2H-pyran-4-yl)-2-(2-((2,2,2-trifluoroethyl)amino)pyridin-4-yl)-1-((2-(trimethylsilyl)ethoxy)methyl)-1H-pyrrolo[3,2-c]pyridin-6-amine. To a mixture of 4-(6-chloro-1-((2-(trimethylsilyl)ethoxy)methyl)-1H-pyrrolo[3,2-c]pyridin-2-yl)-N-(2,2,2-trifluoroethyl)pyridin-2-amine (200 mg, 0.44 mmol) and tetrahydro-2H-pyran-4-amine (132.81 mg, 1.31 mmol) in THE (2 mL) was added Sodium tert-Butoxide (0.44 mL, 0.88 mmol) and Methanesulfonato(2-dicyclohexylphosphino-3,6-dimethoxy-2,4,6-tri-i-propyl-1,1-biphenyl)(2-amino-1,1-biphenyl-2-yl)palladium(II) (39.68 mg, 0.04 mmol). The mixture was stirred at 80° C. for 16 h under nitrogen. LCMS showed 4-(6-chloro-1-((2-(trimethylsilyl)ethoxy)methyl)-1H-pyrrolo[3,2-c]pyridin-2-yl)-N-(2,2,2-trifluoroethyl)pyridin-2-amine was consumed completely and a peak (66%) with desired mass. The mixture was concentrated under reduced pressure to give a residue. The residue was purified by flash silica gel chromatography (Biotage, 12 g SepaFlash® Silica Flash Column, Eluent of 0˜80% Ethyl acetate/Petroleum ether gradient @ 30 mL/min) to give N-(tetrahydro-2H-pyran-4-yl)-2-(2-((2,2,2-trifluoroethyl)amino)pyridin-4-yl)-1-((2-(trimethylsilyl)ethoxy)methyl)-1H-pyrrolo[3,2-c]pyridin-6-amine (150 mg, 0.29 mmol, 65.70% yield) as a yellow solid. MS (ESI): m/z 522.3 [M+1]⁺.

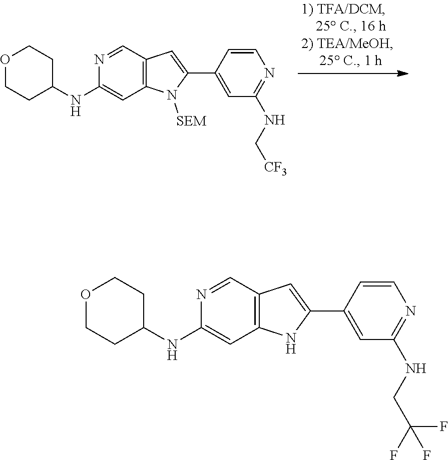 [SMILES: C[Si](C)(C)CCOCn1c(-c2ccnc(NCC(F)(F)F)c2)cc2cnc(NC3CCOCC3)cc21 ; FC(F)(F)CNc1cc(-c2cc3cnc(NC4CCOCC4)cc3[nH]2)ccn1]

N-(Tetrahydro-2H-pyran-4-yl)-2-(2-((2,2,2-trifluoroethyl)amino)pyridin-4-yl)-1H-pyrrolo[3,2-c]pyridin-6-amine. To a mixture of N-(tetrahydro-2H-pyran-4-yl)-2-(2-((2,2,2-trifluoroethyl)amino)pyridin-4-yl)-1-((2-(trimethylsilyl)ethoxy)methyl)-1H-pyrrolo[3,2-c]pyridin-6-amine (150. mg, 0.2900 mmol) in dichloromethane (2 mL) was added Trifluoroacetic acid (2. mL, 0.2900 mmol). The mixture was stirred at 25° C. for 16 h. LCMS showed N-(tetrahydro-2H-pyran-4-yl)-2-(2-((2,2,2-trifluoroethyl)amino)pyridin-4-yl)-1-((2-(trimethylsilyl)ethoxy)methyl)-1H-pyrrolo[3,2-c]pyridin-6-amine was consumed completely and a peak (83%) with the mass of intermediate. The mixture was concentrated under reduced pressure to give a residue. To the residue in Methanol (2 mL) was added triethylamine (2. mL, 0.2900 mmol), the mixture was stirred at 25° C. for 1 h. LCMS showed a peak (94%) with desired mass. The mixture was concentrated under reduced pressure to give a residue. The residue was purified by prep-HPLC (column: Unisil 3-100 C18 Ultra 150*50 mm*3 um; mobile phase: [water (0.225% FA)-ACN]; B %: 5%-35%, 10 min) followed by lyophilization to give N-(tetrahydro-2H-pyran-4-yl)-2-(2-((2,2,2-trifluoroethyl)amino)pyridin-4-yl)-1H-pyrrolo[3,2-c]pyridin-6-amine (102.58 mg, 0.26 mmol, 90.24% yield) (formic acid) as a yellow solid. MS (ESI): m/z 392.3 [M+1]⁺; ¹H NMR (400 MHz, DMSO-d₆) δ=13.37-13.28 (m, 1H), 12.23 (s, 1H), 8.53 (s, 1H), 8.15 (s, 1H), 7.54 (d, J=6.8 Hz, 1H), 7.35 (t, J=6.5 Hz, 1H), 7.12 (d, J=1.3 Hz, 1H), 7.07 (dd, J=1.4, 5.4 Hz, 1H), 7.00 (s, 1H), 6.76 (s, 1H), 4.23-4.21 (m, 2H), 3.93-3.90 (m, 2H), 3.80-3.79 (m, 1H), 3.47-3.41 (m, 2H), 1.95-1.92 (m, 2H), 1.54-1.45 (m, 2H).

### Example 6: N-(2-(2-Methylpyridin-4-yl)-1H-pyrrolo[3,2-c]pyridin-6-yl)cyclopropanecarboxamide

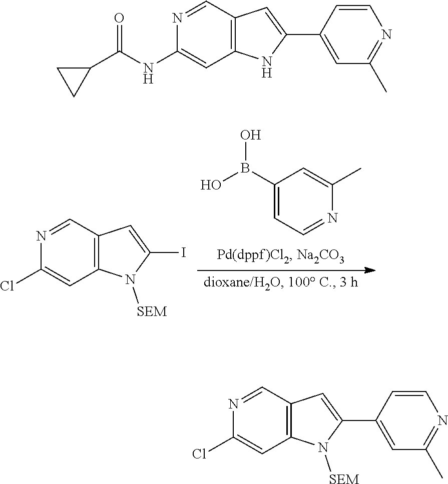

6-Chloro-2-(2-methylpyridin-4-yl)-1-((2-(trimethylsilyl)ethoxy)methyl)-1H-pyrrolo[3,2-c]pyridine. To a mixture of 6-chloro-2-iodo-1-((2-(trimethylsilyl)ethoxy)methyl)-1H-pyrrolo[3,2-c]pyridine (150 g, 366.98 mmol) and (2-methylpyridin-4-yl)boronic acid (52.77 g, 385.33 mmol, 1.05 eq) in dioxane (2000 mL) were added a solution of sodium carbonate (77.79 g, 733.97 mmol) in water (200 mL) followed by [1,1-bis(diphenylphosphino)ferrocene]dichloropall, adium(II) (13.43 g, 18.35 mmol) under nitrogen. The mixture was stirred at 100° C. for 3 h under nitrogen. TLC (Petroleum ether. Ethyl acetate=2:1) showed most of 2-[(6-chloro-2-iodo-pyrrolo[3,2-c]pyridin-1-yl)methoxy]ethyl-trimethyl-silane was consumed and two new spots. The mixture was cooled to 40° C. and concentrated in reduced pressure. The residue was poured into water (500 mL). The aqueous phase was extracted with ethyl acetate (300 mL×3). The combined organic phase was washed with brine (200 mL), dried with anhydrous sodium sulfate, filtered and concentrated in vacuum. The residue was purified by silica gel chromatography (1000 mesh silica gel, Petroleum ether/Ethyl acetate=10/1, 3/1, 1/1) to give 6-chloro-2-(2-methylpyridin-4-yl)-1-((2-(trimethylsilyl)ethoxy)methyl)-1H-pyrrolo[3,2-c]pyridine (70 g, 181.57 mmol, 49.48% yield, 97% purity) as brown solid and recovered 6-chloro-2-iodo-1-((2-(trimethylsilyl)ethoxy)methyl)-1H-pyrrolo[3,2-c]pyridine (59 g, 144.35 mmol, 39.33% yield, 100% purity) as yellow solid. ¹H NMR (400 MHz, CDCl₃) 8.73-8.68 (m, 1H), 8.62 (d, J=5.1 Hz, 1H), 7.47-7.42 (m, 2H), 7.38 (d, J=5.0 Hz, 1H), 6.77 (s, 1H), 5.40 (s, 2H), 3.60-3.52 (m, 2H), 2.65 (s, 3H), 0.97-0.89 (m, 2H), −0.02 (s, 9H).

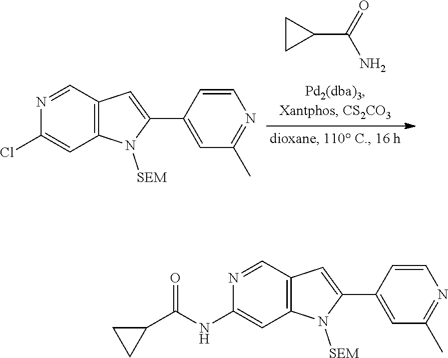 [SMILES: NC(=O)C1CC1 ; Cc1cc(-c2cc3cnc(Cl)cc3n2COCC[Si](C)(C)C)ccn1 ; Cc1cc(-c2cc3cnc(NC(=O)C4CC4)cc3n2COCC[Si](C)(C)C)ccn1]

N-(2-(2-Methylpyridin-4-yl)-1-((2-(trimethylsilyl)ethoxy)methyl)-1H-pyrrolo[3,2-c]pyridin-6-yl)cyclopropanecarboxamide. To a solution of 6-chloro-2-(2-methylpyridin-4-yl)-1-((2-(trimethylsilyl)ethoxy)methyl)-1H-pyrrolo[3,2-c]pyridine (65 g, 173.82 mmol), Cesium carbonate (169.90 g, 521.46 mmol) and cyclopropane carboxamide (29.59 g, 347.64 mmol) in dioxane (700 mL) were added tris(dibenzylideneacetone)dipalladium (11.94 g, 13.04 mmol) and Xantphos 4,5-bis(diphenylphosphino)-9,9-dimethylxanthene (7.54 g, 13.04 mmol) under nitrogen. The mixture was stirred at 110° C. for 16 hr under nitrogen. TLC (Petroleum ether:Ethyl acetate=1:1) showed 6-chloro-2-(2-methylpyridin-4-yl)-1-((2-(trimethylsilyl)ethoxy)methyl)-1H-pyrrolo[3,2-c]pyridine was consumed and two new spots. The reaction mixture was cooled and diluted with 500 mL of ethyl acetate (combined another batch of 6 crude product). The resulting mixture was filtered and the filtrate was concentrated. The residue was purified by silica gel chromatography (1000 mesh silica gel, Petroleum ether/Ethyl acetate=3/1, 1/1, 1/2) to give 90 g of crude product. 90 g of crude product was triturated with 100 mL of methanol to give 24 g of pure product and 66 g of crude product. 66 g of crude product was triturated with 60 mL of methanol to give 24 g of pure product and 42 g of crude product. 42 g of crude product was triturated with 40 mL of methanol to give 10 g of pure product and 32 g of crude product. Three batches of product were combined to give N-(2-(2-methylpyridin-4-yl)-1-((2-(trimethylsilyl)ethoxy)methyl)-1H-pyrrolo[3,2-c]pyridin-6-yl)cyclopropanecarboxamide (58 g, 137.25 mmol, 73.79% yield, 100% purity) as off-white solid. 32 g of crude product was purified by prep-HPLC (column: Phenomenex luna C18 (250*70 mm, 10 um);mobile phase: [water(0.225% FA)-ACN];B %: 28%-58%,19 min) to give N-(2-(2-methylpyridin-4-yl)-1-((2-(trimethylsilyl)ethoxy)methyl)-1H-pyrrolo[3,2-c]pyridin-6-yl)cyclopropanecarboxamide (12 g, 26.41 mmol, 14.20% yield, 93% purity) as yellow solid. ¹H NMR (400 MHz, CDCl₃) 9.05 (s, 1H), 8.61 (d, J=0.6 Hz, 1H), 8.59 (d, J=5.1 Hz, 1H), 8.47 (s, 1H), 7.49 (s, 1H), 7.46-7.41 (m, 1H), 6.76 (s, 1H), 5.44 (s, 2H), 3.70-3.62 (m, 2H), 2.63 (s, 3H), 1.67-1.58 (m, 1H), 1.17-1.09 (m, 2H), 0.99-0.93 (m, 2H), 0.92-0.85 (m, 2H), -0.03 (s, 9H).

Batch 1: To a solution of N-(2-(2-methylpyridin-4-yl)-1-((2-(trimethylsilyl)ethoxy)methyl)-1H-pyrrolo[3,2-c]pyridin-6-yl)cyclopropanecarboxamide (64 g, 151.45 mmol) in dichloromethane (500 mL) and MeOH (50 mL) was added SiliaMetS Thiol (25 g)(SiliaMetS Thiol CAS #1189056-65-2). The mixture was stirred at 25° C. for 6 h. The mixture was filtered. To the filtrate was added SiliaMetS Thiol (25 g) and the mixture was stirred at 25° C. for 16 h. The mixture was filtered. To the filtrate was added SiliaMetS Thiol (25 g) and the mixture was stirred at 25° C. for 48 h. The mixture was filtered and the filtrate was concentrated under vacuum to give N-(2-(2-methylpyridin-4-yl)-1-((2-(trimethylsilyl)ethoxy)methyl)-1H-pyrrolo[3,2-c]pyridin-6-yl)cyclopropanecarboxamide (60 g, 141.98 mmol, 93.75% yield, 100% purity) as off-white solid.

Batch 2: To a solution of N-(2-(2-methylpyridin-4-yl)-1-((2-(trimethylsilyl)ethoxy)methyl)-1H-pyrrolo[3,2-c]pyridin-6-yl)cyclopropanecarboxamide (12 g, 26.41 mmol) in dichloromethane (100 mL) and MeOH (10 mL) was added SiliaMetS Thiol (5 g)(SiliaMetS Thiol CAS #1189056-65-2). The mixture was stirred at 25° C. for 16 h. The mixture was filtered. To the filtrate was added SiliaMetS Thiol (5 g) and the mixture was stirred at 25° C. for 48 h. The mixture was filtered. To the filtrate was added SiliaMetS Thiol (5 g) and the mixture was stirred at 25° C. for 4 h. The mixture was filtered and the filtrate was concentrated under vacuum to give N-(2-(2-methylpyridin-4-yl)-1-((2-(trimethylsilyl)ethoxy)methyl)-1H-pyrrolo[3,2-c]pyridin-6-yl)cyclopropanecarboxamide (9.6 g, 22.72 mmol, 86.02% yield, 100% purity) as off-white solid.

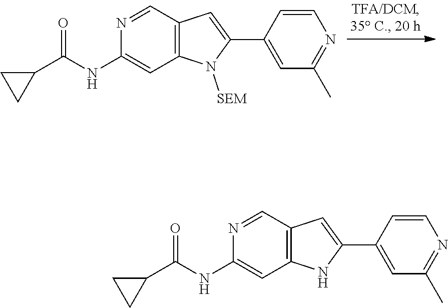 [SMILES: Cc1cc(-c2cc3cnc(NC(=O)C4CC4)cc3n2COCC[Si](C)(C)C)ccn1 ; Cc1cc(-c2cc3cnc(NC(=O)C4CC4)cc3[nH]2)ccn1]

N-(2-(2-Methylpyridin-4-yl)-1H-pyrrolo[3,2-c]pyridin-6-yl)cyclopropanecarboxamide. Batch 1: To a solution of N-(2-(2-methylpyridin-4-yl)-1-((2-(trimethylsilyl)ethoxy)methyl)-1H-pyrrolo[3,2-c]pyridin-6-yl)cyclopropanecarboxamide (9.6 g, 22.72 mmol) in dichloromethane (30 mL) was added trifluoroacetic acid (30 mL) at 0° C. The mixture was stirred at 25° C. for 20 h. LCMS showed material was consumed, the desired mass and the mass of N-(1-(hydroxymethyl)-2-(2-methylpyridin-4-yl)-1H-pyrrolo[3,2-c]pyridin-6-yl)cyclopropanecarboxamide. The mixture was concentrated under vacuum. The residue was dissolved with methanol (60 mL) and the solution was adjusted to pH=9 with triethylamine. The resulting mixture was stirred at 40° C. for 1 h. LCMS showed one peak with desired mass. White solid was precipitated. The mixture was filter and the filter cake was concentrated under vacuum. The filter cake was collected to give N-(2-(2-methylpyridin-4-yl)-1H-pyrrolo[3,2-c]pyridin-6-yl)cyclopropanecarboxamide (5.7 g, 19.50 mmol, 85.83% yield) as white solid.

Batch 2: To a solution of N-(2-(2-methylpyridin-4-yl)-1-((2-(trimethylsilyl)ethoxy)methyl)-1H-pyrrolo[3,2-c]pyridin-6-yl)cyclopropanecarboxamide (60.00 g, 141.98 mmol) in dichloromethane (200 mL) was added trifluoroacetic acid (200 mL) at 0° C. The mixture was stirred at 35° C. for 20 h. LCMS showed material was consumed, the desired mass and the mass of N-(1-(hydroxymethyl)-2-(2-methylpyridin-4-yl)-1H-pyrrolo[3,2-c]pyridin-6-yl) cyclopropane carboxamide. The mixture was concentrated under vacuum. The residue was dissolved with methanol (60 mL) and the solution was adjusted to pH=9 with triethylamine. The resulting mixture was stirred at 40° C. for 1 h. LCMS showed one peak with desired mass. White solid was precipitated. The mixture was filter and the filter cake was concentrated under vacuum to give N-(2-(2-methylpyridin-4-yl)-1H-pyrrolo[3,2-c]pyridin-6-yl)cyclopropanecarboxamide (40 g, 136.83 mmol, 96.37% yield) as white solid. HNMR contain methanol. The filtrate was concentrated under vacuum. The residue was poured into saturated sodium bicarbonate (300 mL). The aqueous phase was extracted with dichloromethane (200 mL×3). The combined organic phase was dried with anhydrous sodium sulfate, filtered and concentrated in vacuum to give N-(2-(2-methylpyridin-4-yl)-1H-pyrrolo[3,2-c]pyridin-6-yl)cyclopropanecarboxamide (24 g, crude) as yellow solid.

For purification: a solution of N-(2-(2-methylpyridin-4-yl)-1H-pyrrolo[3,2-c]pyridin-6-yl)cyclopropanecarboxamide (45.7 g, 156.33 mmol) in methanol (700 mL) at 70° C. for 1 h to get a yellow solution. The solution was cooled to room temperature slowly in the oil bath. White solid formed. The suspension was filtered and the filter cake was dried under vacuum, but HNMR contain methanol. The solid was purified by recrystallization by ethanol to give N-(2-(2-methylpyridin-4-yl)-1H-pyrrolo[3,2-c]pyridin-6-yl)cyclopropanecarbox amide (23.71 g, 81.02 mmol, 51.83% yield, 99.9% purity) as white solid. The mother liquid was concentrated and recrystallization by ethanol again to give N-[2-(2-methyl-4-pyridyl)-1H-pyrrolo[3,2-c]pyridin-6-yl]cyclopropanecarboxamide (11.11 g, 37.97 mmol, 24.29% yield, 99.9% purity) as white solid. MS (ESI): m/z 293.1 [M+1]⁺; ¹H NMR (400 MHz, DMSO-d₆) 11.99 (br s, 1H), 10.66 (s, 1H), 8.61 (s, 1H), 8.49 (d, J=5.3 Hz, 1H), 8.20 (s, 1H), 7.69 (s, 1H), 7.60 (d, J=4.5 Hz, 1H), 7.22 (s, 1H), 2.52 (s, 3H), 2.13-1.94 (m, 1H), 0.90-0.69 (m, 4H).

WXWCGHCR-001-14-B2: ¹H NMR (400 MHz, DMSO-d₆) 12.00 (s, 1H), 10.68 (s, 1H), 8.61 (s, 1H), 8.49 (d, J=5.3 Hz, 1H), 8.19 (s, 1H), 7.70 (s, 1H), 7.64-7.57 (m, 1H), 7.23 (s, 1H), 2.52 (s, 3H), 2.08-1.98 (m, 1H), 0.88-0.73 (m, 4H).

### Example 7: (R)-1-Methyl-N-(2-(2-((1,1,1-trifluoropropan-2-yl)amino)pyrimidin-4-yl)-1H-pyrrolo[3,2-c]pyridin-6-yl)-1H-pyrazole-4-carboxamide

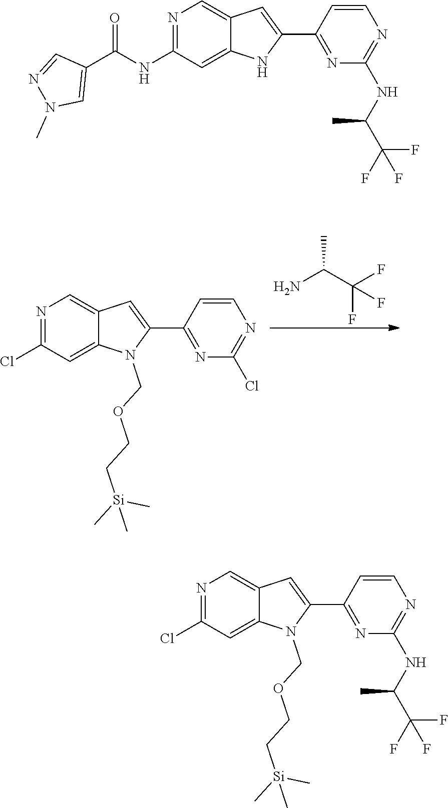

(R)-4-(6-Chloro-1-((2-(trimethylsilyl)ethoxy)methyl)-1H-pyrrolo[3,2-c]pyridin-2-yl)-N-(1,1,1-trifluoropropan-2-yl)pyrimidin-2-amine. 6-chloro-2-(2-chloropyrimidin-4-yl)-1-((2-(trimethylsilyl)ethoxy)methyl)-1H-pyrrolo[3,2-c]pyridine (500 mg, 1.265 mmol) and (R)-1,1,1-trifluoropropan-2-amine (2500 mg, 22.11 mmol) were combined in 2-Propanol (10 mL) and allowed to stir at 80° C. overnight in a 30 mL capped vial. 1.5 g additional of the amine was added then placed in microwave at 150° C. for 2 h. Additional 1.5 g of the amine was added and heated in the microwave for 2 additional hour at 150° C. Additional 0.5 g of the amine was added again and heated in the microwave for 2 additional h at 150° C. The reaction mixture was concentrated under reduced pressure to give an oil. The oil was purified on a Biotage SNAP silica column to afford (R)-4-(6-chloro-1-((2-(trimethylsilyl)ethoxy)methyl)-1H-pyrrolo[3,2-c]pyridin-2-yl)-N-(1,1,1-trifluoropropan-2-yl)pyrimidin-2-amine (254 mg, 0.538 mmol, 42.6% yield).

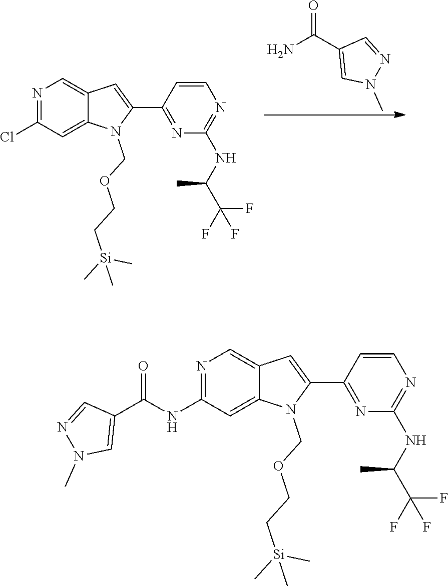

(R)-1-Methyl-N-(2-(2-((1,1,1-trifluoropropan-2-yl)amino)pyrimidin-4-yl)-1-((2-(trimethylsilyl)ethoxy)methyl)-1H-pyrrolo[3,2-c]pyridin-6-yl)-1H-pyrazole-4-carboxamide. (R)-4-(6-chloro-1-((2-(trimethylsilyl)ethoxy)methyl)-1H-pyrrolo[3,2-c]pyridin-2-yl)-N-(1,1,1-trifluoropropan-2-yl)pyrimidin-2-amine (254 mg, 0.538 mmol), 1-methyl-1H-pyrazole-4-carboxamide (94 mg, 0.753 mmol), tris(dibenzylideneacetone)dipalladium(0) (99 mg, 0.108 mmol), and xantphos (62.3 mg, 0.108 mmol) were combined in Dioxane (3.191 mL) and heated to 95° C. overnight in a 16 mL screw-capped vial. The reaction mixture was diluted with ethyl acetate and filtered through a pad of Celite. The filtrate was concentrated and purified on a Biotage SNAP silica column to afford (R)-1-methyl-N-(2-(2-((1,1,1-trifluoropropan-2-yl)amino)pyrimidin-4-yl)-1-((2-(trimethylsilyl)ethoxy)methyl)-1H-pyrrolo[3,2-c]pyridin-6-yl)-1H-pyrazole-4-carboxamide (181 mg, 0.323 mmol, 60.0% yield).

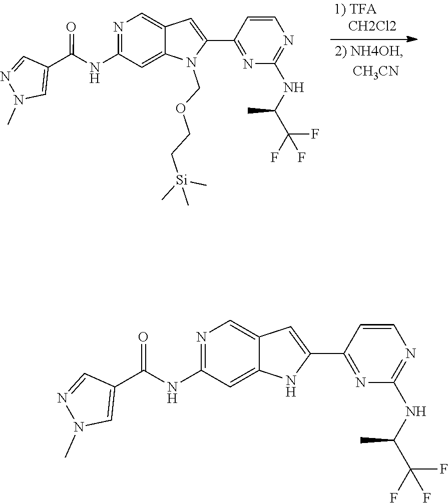 [SMILES: C[C@@H](Nc1nccc(-c2cc3cnc(NC(=O)c4cnn(C)c4)cc3n2COCC[Si](C)(C)C)n1)C(F)(F)F ; C[C@@H](Nc1nccc(-c2cc3cnc(NC(=O)c4cnn(C)c4)cc3[nH]2)n1)C(F)(F)F]

(R)-1-Methyl-N-(2-(2-((1,1,1-trifluoropropan-2-yl)amino)pyrimidin-4-yl)-1H-pyrrolo[3,2-c]pyridin-6-yl)-1H-pyrazole-4-carboxamide. (R)-1-methyl-N-(2-(2-((1,1,1-trifluoropropan-2-yl)amino)pyrimidin-4-yl)-1-((2-(trimethylsilyl)ethoxy)methyl)-1H-pyrrolo[3,2-c]pyridin-6-yl)-1H-pyrazole-4-carboxamide (181 mg, 0.323 mmol) was dissolved in Dichloromethane (10 mL) and to this mixture was added TFA (10 ml, 130 mmol). The resulting mix was stirred capped but vented at ambient temperature overnight. The reaction mixture was concentrated to an oil under reduced pressure and redissolved in acetonitrile (10 mL). Saturated aqueous solution of ammonium hydroxide (6 ml, 154 mmol) was added and let mix at ambient temperature. After stirring at ambient temperature for 1 h, the solids were filtered, rinsed well with water, and then dried in a vacuum oven for a few hours at 45° C. to afford (R)-1-methyl-N-(2-(2-((1,1,1-trifluoropropan-2-yl)amino)pyrimidin-4-yl)-1H-pyrrolo[3,2-c]pyridin-6-yl)-1H-pyrazole-4-carboxamide (130.7 mg, 0.304 mmol, 94% yield). 1H NMR (400 MHz, DMSO-d₆) δ ppm 11.86-12.01 (m, 1H), 10.38 (s, 1H), 8.66-8.75 (m, 1H), 8.43-8.47 (m, 1H), 8.34-8.42 (m, 2H), 8.11-8.19 (m, 1H), 7.67-7.81 (m, 1H), 7.36-7.42 (m, 1H), 7.23-7.30 (m, 1 H), 5.36-5.61 (m, 1H), 3.88 (s, 3H), 1.33-1.41 (m, 3H).

### Example 8: N-(Cyclopropylmethyl)-2-(2-methylpyridin-4-yl)-1H-pyrrolo[3,2-c]pyridin-6-amine

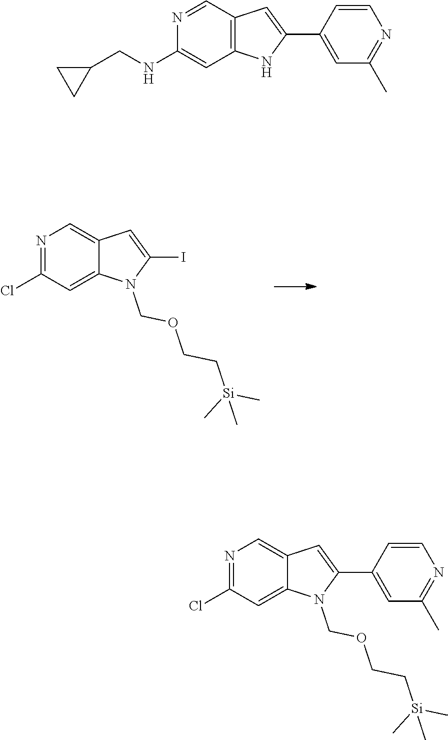 [SMILES: Cc1cc(-c2cc3cnc(NCC4CC4)cc3[nH]2)ccn1 ; C[Si](C)(C)CCOCn1c(I)cc2cnc(Cl)cc21 ; Cc1cc(-c2cc3cnc(Cl)cc3n2COCC[Si](C)(C)C)ccn1]

2-[[6-Chloro-2-(2-methyl-4-pyridyl)pyrrolo[3,2-c]pyridin-1-yl]methoxy]ethyl-trimethyl-silane. To 2-[(6-chloro-2-iodo-pyrrolo[3,2-c]pyridin-1-yl)methoxy]ethyl-trimethyl-silane (4000. mg, 9.79 mmol), 2-methylpyridine-4-boronic acid (1340.12 mg, 9.79 mmol), Dichloro 1,1′-bis(diphenylphosphino)ferrocene palladium (II) (1201.72 mg, 1.47 mmol) and Sodium carbonate (4227.78 mg, 39.14 mmol) was added 1,4-Dioxane (18 mL) and Water (6 mL). It's flushed by N2 then sealed and stirred at 85° C. for 16h. It's worked up (50mlEAX3/50 ml water). the EA layers were combined and concentrated then purified by Biotage silica (0-100% EA/Hex) to get 2-[[6-chloro-2-(2-methyl-4-pyridyl)pyrrolo[3,2-c]pyridin-1-yl]methoxy]ethyl-trimethyl-silane (1400 mg, 3.7438 mmol, 38.256% yield).

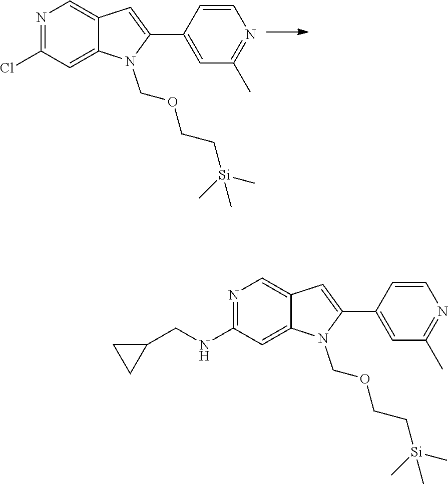 [SMILES: Cc1cc(-c2cc3cnc(Cl)cc3n2COCC[Si](C)(C)C)ccn1 ; Cc1cc(-c2cc3cnc(NCC4CC4)cc3n2COCC[Si](C)(C)C)ccn1]

N-(Cyclopropylmethyl)-2-(2-methyl-4-pyridyl)-1-(2-trimethylsilylethoxymethyl)pyrrolo[3,2-c]pyridin-6-amine. To the mixture of 2-[[6-chloro-2-(2-methyl-4-pyridyl)pyrrolo[3,2-c]pyridin-1-yl]methoxy]ethyl-trimethyl-silane (200. mg, 0.5300 mmol), BrettPhos Pd G1Methyl-t-Butyl Ether Adduct (64.08 mg, 0.0800 mmol), RUPHOS (37.44 mg, 0.0800 mmol), Cesium carbonate (350.67 mg, 1.07 mmol), and Cyclopropylmethanamine (0.09 mL, 1.07 mmol) was added 1,4-Dioxane (5 mL). It's stirred at 100° C. for 16h. It's purified by Biotage silica (0-100% EA/Hex) to get N-(cyclopropylmethyl)-2-(2-methyl-4-pyridyl)-1-(2-trimethylsilylethoxymethyl)pyrrolo[3,2-c]pyridin-6-amine (60 mg, 0.1468 mmol, 27.455% yield).

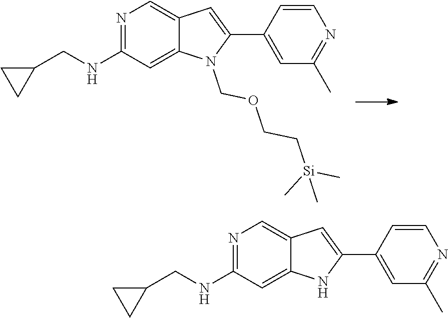

N-(Cyclopropylmethyl)-2-(2-methylpyridin-4-yl)-1H-pyrrolo[3,2-c]pyridin-6-amine. To N-(cyclopropylmethyl)-2-(2-methyl-4-pyridyl)-1-(2-trimethylsilylethoxymethyl)pyrrolo[3,2-c]pyridin-6-amine (60. mg, 0.1500 mmol) in DCM (5 mL) was added Trifluoroacetic acid (3. mL, 39.18 mmol). It's stirred at r.t for 16h. It's concentrated and dissolved in 5 ml MeOH and 7M Ammonia (0.2 mL, 1.4 mmol) was added. It's stirred at rt for 6h. It's concentrated and purified by Biotage C18 (0-20% ACN/0.1% FA in water). The right fractions were combined and passed through Strata strong Cation column. The Strata strong cation column was washed by 30 ml 5% NH4OH in MeOH. The MeOH solution was concentrated to get N-(cyclopropylmethyl)-2-(2-methyl-4-pyridyl)-1H-pyrrolo[3,2-c]pyridin-6-amine (6 mg, 0.0216 mmol, 14.68% yield).

### Example 9: 4-(6-((1-Methyl-1h-pyrazol-4-yl)amino)-1h-pyrrolo[3,2-c]pyridin-2-yl)picolinonitrile

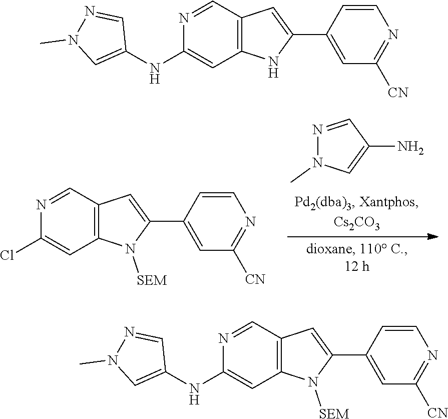

4-(6-((1-Methyl-1H-pyrazol-4-yl)amino)-1-((2-(trimethylsilyl)ethoxy)methyl)-1H-pyrrolo[3,2-c]pyridin-2-yl)picolinonitrile. To a solution of 4-(6-chloro-1-((2-(trimethylsilyl)ethoxy)methyl)-1H-pyrrolo[3,2-c]pyridin-2-yl)picolinonitrile (200 mg, 0.52 mmol) and 1-methyl-1H-pyrazol-4-amine (126.15 mg, 1.30 mmol) in dioxane (4 mL) was added cesium carbonate (338.57 mg, 1.04 mmol), Tris(dibenzylideneacetone)dipalladium (47.58 mg, 0.05 mmol) and 4,5-Bis(diphenylphosphino)-9,9-dimethylxanthene (60.13 mg, 0.10 mmol). The mixture was stirred under nitrogen atmosphere at 110° C. for 12 h. LCMS showed the reaction was completed and the desired mass was detected. The reaction mixture was filtered and the filtrate was concentrated to give the crude product. The crude product was purified by prep-TLC (silicon dioxide, Ethyl acetate:methanol=10:1) to give 4-(6-((1-methyl-1H-pyrazol-4-yl)amino)-1-((2-(trimethylsilyl)ethoxy)methyl)-1H-pyrrolo[3,2-c]pyridin-2-yl)picolinonitrile (150 mg, 0.34 mmol, 64.79% yield) as a yellow solid. MS (ESI): m/z 446.1 [M+1]⁺.

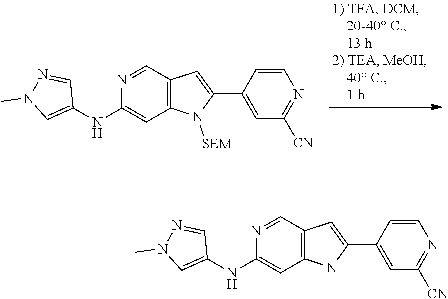

4-(6-((1-Methyl-1H-pyrazol-4-yl)amino)-1H-pyrrolo[3,2-c]pyridin-2-yl)picolinonitrile. The mixture of 4-(6-((1-methyl-1H-pyrazol-4-yl)amino)-1-((2-(trimethylsilyl)ethoxy)methyl)-1H-pyrrolo[3,2-c]pyridin-2-yl)picolinonitrile (150 mg, 336.63 mol) in dichloromethane (1.5 mL) and trifluoroacetic acid (1.5 mL) was stirred at 40° C. for 1 h. LCMS showed the staring material remained and the desired mass was detected. The reaction mixture was stirred at 20° C. for another 12 h. LCMS showed 4-(6-((1-methyl-1H-pyrazol-4-yl)amino)-1-((2-(trimethylsilyl)ethoxy)methyl)-1H-pyrrolo[3,2-c]pyridin-2-yl)picolinonitrile was consumed completely. The mixture was concentrated under reduced pressure to give the residue. The residue was added methanol (1.5 mL) and triethylamine (373.68 mg, 3.69 mmol, 0.5 mL). The mixture was stirred at 40° C. for another 1 h. LCMS showed the reaction was completed and the desired mass was detected. The reaction mixture was concentrated to give the crude product. The crude product was purified by prep-HPLC (formic acid condition, column: Phenomenex Luna C18 100*30 mm*5 um; mobile phase: [water(0.2% FA)-ACN];B %: 5%-30%, 10 min) to give 4-(6-((1-methyl-1H-pyrazol-4-yl)amino)-1H-pyrrolo[3,2-c]pyridin-2-yl)picolinonitrile(53.54 mg, 0.14 mmol, 41.73% yield, 94.81% purity, formic acid) as a yellow solid. ¹H NMR (400 MHz, DMSO-d₆) 11.82 (br s, 1H), 8.76 (d, J=5.1 Hz, 1H), 8.57-8.52 (m, 2H), 8.44 (d, J=1.1 Hz, 1H), 8.13 (s, 0.3H), 8.04 (dd, J=1.8, 5.3 Hz, 1H), 7.88 (s, 1H), 7.46-7.38 (m, 2H), 6.62 (s, 1H), 3.82 (s, 3H). MS (ESI): m/z 316.2 [M+1]⁺.

### Example 10: N-(Tetrahydro-2H-pyran-4-yl)-2-(2-((2,2,2-trifluoroethyl)amino)pyrimidin-4-yl)-1H-pyrrolo[3,2-c]pyridin-6-amine

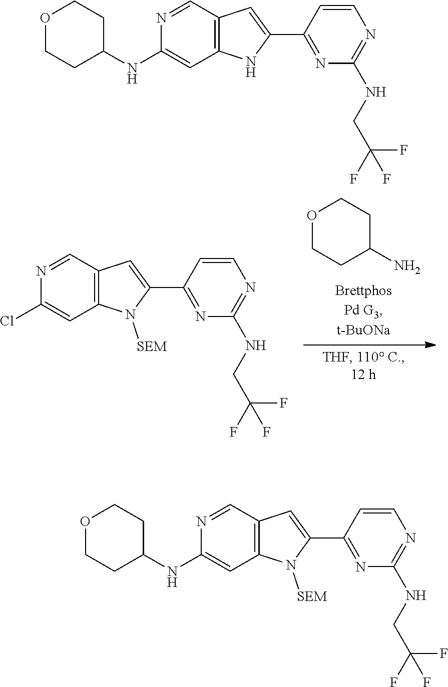

N-(Tetrahydro-2H-pyran-4-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine. This reaction was paralleled for two batches: To a solution of 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine (100 mg, 218.36 μmol) and tetrahydro-2H-pyran-4-amine (33.13 mg, 327.55 μmol) in THF (5 mL) was added sodiumtert-butoxide (2 M, 272.95 μL) and [2-(2-aminophenyl)phenyl]-methylsulfonyloxy-palladium; dicyclohexyl-[3,6-dimethoxy-2-(2,4,6-triisopropylphenyl)phenyl]phosphane (19.79 mg, 21.84 μmol). The mixture was stirred under argon atmosphere at 110° C. for 12 h. LCMS showed the reaction was complete and the desired mass was detected. The two batches reaction mixture was concentrated together to give the crude product. The crude product was purified by prep-TLC (Petroleum ether:Ethyl acetate=0:1) to give N-(tetrahydro-2H-pyran-4-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine (210 mg, 401.81 μmol, 92.00% yield) as a green solid. MS (ESI): m/z 523.2 [M+1]⁺.

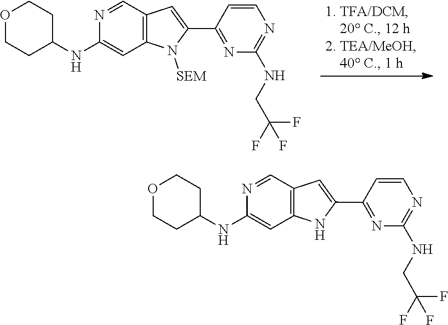

N-(Tetrahydro-2H-pyran-4-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine. The mixture of N-(tetrahydro-2H-pyran-4-yl)-2-(2-((2,2,2-trifluoroethyl)amino)pyrimidin -4-yl)-1-((2-(trimethylsilyl)ethoxy)methyl)-1H-pyrrolo[3,2-c]pyridin-6-amine (210 mg, 401.81 μmol) in dichloromethane (2 mL) and trifluoroacetic acid (2 mL) was stirred at 25° C. for 12 h. LCMS showed N-(tetrahydro-2H-pyran-4-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine was consumed completely. The mixture was concentrated under reduced pressure to remove dichloromethane. To the residue was added methanol (2 mL) and triethylamine (363.50 mg, 3.59 mmol, 500 μL). The mixture was stirred at 40° C. for another 1 h. LCMS showed the reaction was complete and the desired mass was detected. The reaction mixture was concentrated to give the crude product. The crude product purified by prep-HPLC (FA condition, column: Phenomenex Synergi C18 150*25*10 um; mobile phase: [water(0.2% FA)-ACN]; B %: 10%-30%, 10 min), then it was purified again by prep-HPLC (FA condition, column: Phenomenex Synergi C18 150*25*10 um; mobile phase: [water(0.2% FA)-ACN]; B %: 15%-35%, 8 min), then it was purified again by prep-HPLC (neutral condition, column: Waters Xbridge BEH C18 100*25 mm*5 um; mobile phase: [water(10 mM NH₄HCO₃)-ACN]; B %: 20%-60%, 8 min) to give N-(tetrahydro-2H-pyran-4-yl)-2-(2-((2,2,2-trifluoroethyl)amino)pyrimidin-4-yl)-1H-pyrrolo[3,2-c]pyridin-6-amine (33.45 mg, 84.98 μmol, 21.15% yield, 99% purity) as a yellow solid. ¹H NMR (400 MHz, DMSO-d₆) 11.20 (s, 1H), 8.40 (s, 1H), 8.30 (d, J=5.0 Hz, 1H), 7.64 (s, 1H), 7.20 (s, 1H), 7.17 (d, J=5.3 Hz, 1H), 6.38 (s, 1H), 6.09 (d, J=7.9 Hz, 1H), 4.48-4.27 (s, 2H), 3.94-3.76 (m, 3H), 3.45-3.37 (m, 2H), 1.92-1.87 (m, 2H), 1.53-1.36 (m, 2H). MS (ESI): m/z 393.3 [M+1]⁺.

### Example 11: 4-(6-((3-(Tetrahydro-2h-pyran-4-yl)phenyl)amino)-1H-pyrrolo[3,2-c]pyridin-2-yl)picolinonitrile

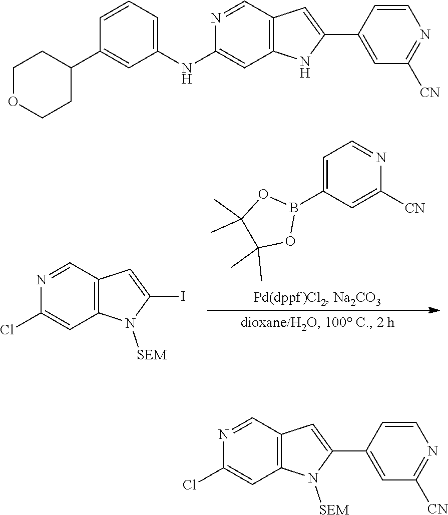 [SMILES: N#Cc1cc(-c2cc3cnc(Nc4cccc(C5CCOCC5)c4)cc3[nH]2)ccn1 ; C[Si](C)(C)CCOCn1c(I)cc2cnc(Cl)cc21 ; CC1(C)OB(c2ccnc(C#N)c2)OC1(C)C ; C[Si](C)(C)CCOCn1c(-c2ccnc(C#N)c2)cc2cnc(Cl)cc21]

4-(6-Chloro-1-((2-(trimethylsilyl)ethoxy)methyl)-1H-pyrrolo[3,2-c]pyridin-2-yl)picolinonitrile. To a solution of 4-(4,4,5,5-tetramethyl-1,3,2-dioxaborolan-2-yl)picolinonitrile (185.75 mg, 0.8 mmol) and 6-chloro-2-iodo-1-((2-(trimethylsilyl)ethoxy)methyl)-1H-pyrrolo[3,2-c]pyridine (300.00 mg, 0.74 mmol) in dioxane (5 mL) and water (0.5 mL) was added [1,1-Bis(diphenylphosphino)ferrocene]dichloropalladium(II) (53.71 mg, 0.07 mmol) and sodium carbonate (194.48 mg, 1.83 mmol). The mixture was stirred at 100° C. for 2 h under nitrogen atmosphere. TLC (Petroleum ether:Ethyl acetate=1:1) showed 6-chloro-2-iodo-1-((2-(trimethylsilyl)ethoxy)methyl)-1H-pyrrolo[3,2-c]pyridine was consumed completely and some new spots was formed. The reaction mixture was concentrated to give the crude product. The crude product was purified by prep-TLC (Petroleum ether:Ethyl acetate=1:1) to give 4-(6-chloro-1-((2-(trimethylsilyl)ethoxy)methyl)-1H-pyrrolo[3,2-c]pyridin-2-yl)picolinonitrile (140 mg, 0.36 mmol, 49.55% yield) as a yellow solid. MS (ESI): m/z 385.1 [M+1]⁺.

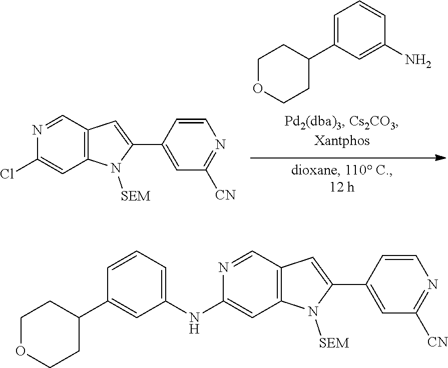 [SMILES: Nc1cccc(C2CCOCC2)c1 ; C[Si](C)(C)CCOCn1c(-c2ccnc(C#N)c2)cc2cnc(Cl)cc21 ; C[Si](C)(C)CCOCn1c(-c2ccnc(C#N)c2)cc2cnc(Nc3cccc(C4CCOCC4)c3)cc21]

-(6-((3-(Tetrahydro-2H-pyran-4-yl)phenyl)amino)-1-((2-(trimethylsilyl)ethoxy)methyl)-1H-pyrrolo[3,2-c]pyridin-2-yl)picolinonitrile. To a solution of 4-(6-chloro-1-((2-(trimethylsilyl)ethoxy)methyl)-1H-pyrrolo[3,2-c]pyridin-2-yl)picolinonitrile (200 mg, 0.52 mmol) and 3-(tetrahydro-2H-pyran-4-yl)aniline (138.13 mg, 0.78 mmol) in dioxane (5 mL) was added cesium carbonate (338.57 mg, 1.04 mmol) and 4,5-Bis(diphenylphosphino)-9,9-dimethylxanthene (60.13 mg, 0.10 mmol), Tris(dibenzylideneacetone)dipalladium (47.58 mg, 0.052 mmol). The mixture was stirred under nitrogen atmosphere at 110° C. for 12 h. LCMS showed the reaction was completed and the desired mass was detected. The reaction mixture was concentrated to give the crude product. The crude product was purified by prep-TLC (Petroleum ether:Ethyl acetate=1:1) to give 4-(6-((3-(tetrahydro-2H-pyran-4-yl)phenyl)amino)-1-((2-(trimethylsilyl)ethoxy)methyl)-1H-pyrrolo[3,2-c]pyridin-2-yl)picolinonitrile (210 mg, 0.40 mmol, 76.88% yield) as yellow oil. MS (ESI): m/z 526.2 [M+1]⁺.

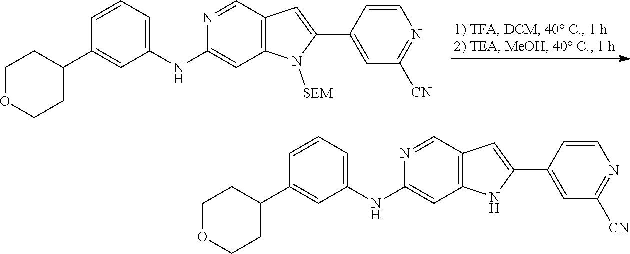

4-(6-((3-(Tetrahydro-2H-pyran-4-yl)phenyl)amino)-1H-pyrrolo[3,2-c]pyridin-2-yl)picolinonitrile. The mixture of 4-(6-((3-(tetrahydro-2H-pyran-4-yl)phenyl)amino)-1-((2-(trimethylsilyl) ethoxy)methyl)-1H-pyrrolo[3,2-c]pyridin-2-yl)picolinonitrile (210 mg, 0.40 mmol) in dichloromethane (2 mL) and trifluoroacetic acid (2 mL) was stirred at 40° C. for 1 h. LCMS showed little 4-(6-((3-(tetrahydro-2H-pyran-4-yl)phenyl)amino)-1-((2-(trimethylsilyl) ethoxy)methyl)-1H-pyrrolo[3,2-c]pyridin-2-yl)picolinonitrile remained. The mixture was concentrated under reduced pressure to remove dichloromethane. The residue was added methanol (2 mL) and triethylamine (404.21 mg, 3.99 mmol, 0.56 mL). The mixture was stirred at 40° C. for another 1 h. LCMS showed the reaction was completed and the desired mass was detected. The reaction mixture was concentrated to give the crude product. The crude product purified by prep-HPLC (formic acid condition, column: Phenomenex Luna C18 200*40 mm*10 um; mobile phase: [water(0.2% FA)-ACN]; B %: 10%-40%, 10 min) to give 4-(6-((3-(tetrahydro-2H-pyran-4-yl)phenyl)amino)-1H-pyrrolo[3,2-c]pyridin-2-yl) picolinonitrile (57.54 mg, 0.13 mmol, 31.52% yield, 99% purity, formic acid) as a yellow solid. ¹H NMR (400 MHz, DMSO-d₆) 11.77 (s, 1H), 8.80-8.73 (m, 2H), 8.60 (s, 1H), 8.45 (d, J=1.1 Hz, 1H), 8.13 (s, 0.3H), 8.06 (dd, J=1.8, 5.3 Hz, 1H), 7.48-7.37 (m, 3H), 7.18 (t, J=7.8 Hz, 1H), 6.85 (s, 1H), 6.75 (d, J=7.8 Hz, 1H), 3.99-3.90 (m, 2H), 3.47-3.34 (m, 2H), 2.77-2.68 (m, 1H), 1.76-1.60 (m, 4H). MS (ESI): m/z 396.3 [M+1]⁺.

### Example 12: N-(3-Methyl-5-(1H-1,2,4-triazol-1-yl)phenyl)-2-(2-((2,2,2-trifluoroethyl)amino)pyrimidin-4-yl)-1H-pyrrolo[3,2-c]pyridin-6-amine

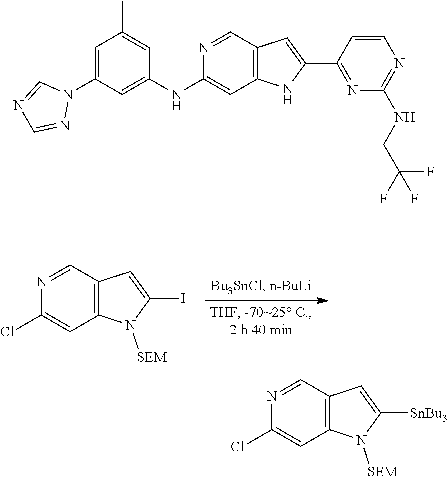

6-Chloro-2-(tributylstannyl)-1-((2-(trimethylsilyl)ethoxy)methyl)-1H-pyrrolo[3,2-c]pyridine. To a mixture of 6-chloro-2-iodo-1-((2-(trimethylsilyl)ethoxy)methyl)-1H-pyrrolo[3,2-c]pyridine (3 g, 7.34 mmol) in THF (45 mL) was added butyllithium (2.5 M, 4.40 mL) dropwise at −70° C., then the mixture was stirred at −70° C. for 10 min. Then tributylchlorostannane (3.34 g, 10.28 mmol, 2.76 mL) was added dropwise at −70° C. The resulting mixture was stirred at 25° C. for 2.5 h. TLC (Petroleum ether:Ethyl acetate=10:1) showed 6-chloro-2-iodo-1-((2-(trimethylsilyl)ethoxy)methyl)-1H-pyrrolo[3,2-c]pyridine was consumed completely and some new spots was formed. The reaction mixture was quenched by saturated ammonium chloride (50 mL), and extracted with ethyl acetate (50×3 mL). The combined organic layers were dried over anhydrous sodium sulfate, filtered, and concentrated under reduced pressure to give a residue. The residue was purified by column chromatography (100-200 mesh silica gel, Petroleum ether:Ethyl acetate=1:0 to 1:0) to give 6-chloro-2-(tributylstannyl)-1-((2-(trimethylsilyl)ethoxy)methyl)-1H-pyrrolo[3,2-c]pyridine (2.87 g, 5.02 mmol, 68.37% yield) as yellow oil. MS (ESI): m/z 573.1 [M+1]⁺.

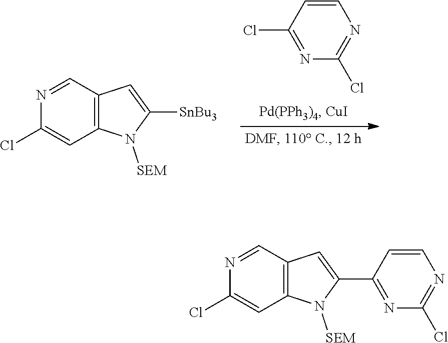

6-Chloro-2-(2-chloropyrimidin-4-yl)-1-((2-(trimethylsilyl)ethoxy)methyl)-1H-pyrrolo[3,2-c]pyridine. To a solution of 6-chloro-2-(tributylstannyl)-1-((2-(trimethylsilyl)ethoxy)methyl)-1H-pyrrolo[3,2-c]pyridine (2.87 g, 5.02 mmol) and 2,4-dichloropyrimidin (822.41 mg, 5.52 mmol) in DMF (40 mL) was added copper iodide (95.58 mg, 0.5 mmol) and Tetrakis(triphenylphosphine) (289.96 mg, 0.025 mmol). The mixture was stirred at 110° C. for 12 h under nitrogen atmosphere. LCMS showed the reaction was completed and the desired mass was detected. The reaction mixture was diluted with water (40 mL) and extracted with ethyl acetate (40 mL×3). The combined organic layers were washed with brine (50 mL×2), dried over anhydrous sodium sulfate, filtered and concentrated under reduced pressure to give a residue. The residue was purified by column chromatography (100-200 mesh silica gel, Petroleum ether:Ethyl acetate=8:1 to 1:1) to give 6-chloro-2-(2-chloropyrimidin-4-yl)-1-((2-(trimethylsilyl)ethoxy)methyl)-1H-pyrrolo[3,2-c]pyridine (1.35 g, 3.41 mmol, 68.04% yield) as a yellow solid. MS (ESI): m/z 395.0 [M+1]⁺. H NMR (400 MHz, CDCl₃) 8.78 (s, 1H), 8.68 (d, J=5.4 Hz, 1H), 7.70 (d, J=5.3 Hz, 2H), 7.54 (s, 1H), 6.07 (s, 2H), 3.62-3.58 (m, 2H), 0.92-0.88 (m, 2H), -0.08 (s, 9H).

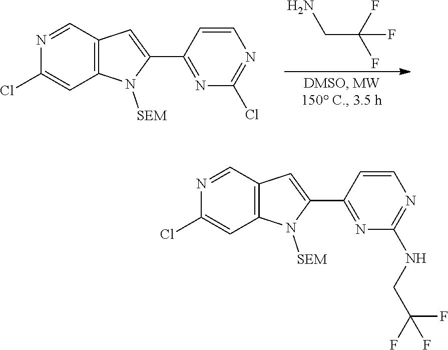 [SMILES: C[Si](C)(C)CCOCn1c(-c2ccnc(Cl)n2)cc2cnc(Cl)cc21 ; NCC(F)(F)F ; C[Si](C)(C)CCOCn1c(-c2ccnc(NCC(F)(F)F)n2)cc2cnc(Cl)cc21]

4-(6-Chloro-1-((2-(trimethylsilyl)ethoxy)methyl)-1H-pyrrolo[3,2-c]pyridin-2-yl)-N-(2,2,2-trifluoroethyl)pyrimidin-2-amine. This reaction was paralleled for four batches: The mixture of 6-chloro-2-(2-chloropyrimidin-4-yl)-1-((2-(trimethylsilyl)ethoxy)methyl)-1H-pyrrolo[3,2-c]pyridine (175 mg, 0.44 mmol) and 2,2,2-trifluoroethanamine (1.65 g, 16.70 mmol, 1.31 mL) in DMSO (3 mL) was stirred at 150° C. for 3.5 h under microwave. LCMS showed the reaction was completed and the desired mass was detected. The four batches reaction mixture was added to water (20 mL) together, extracted with ethyl acetate (15 mL×3). The organic layer was dried over anhydrous sodium sulfate, concentrated under vacuum to give the crude product. The crude product was purified by prep-TLC (Petroleum ether:Ethyl acetate=1:1) to give 4-(6-chloro-1-((2-(trimethylsilyl)ethoxy)methyl)-1H-pyrrolo[3,2-c]pyridine-2-yl)-N-(2,2,2-trifluoroethyl)pyrimidin-2-amine (690 mg, 1.51 mmol, 85.10% yield) as a yellow solid. MS (ESI): m/z 458.1 [M+1]⁺.

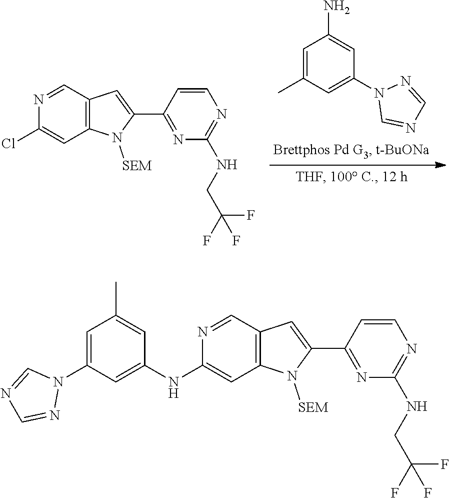 [SMILES: C[Si](C)(C)CCOCn1c(-c2ccnc(NCC(F)(F)F)n2)cc2cnc(Cl)cc21 ; Cc1cc(N)cc(-n2cncn2)c1 ; Cc1cc(Nc2cc3c(cn2)cc(-c2ccnc(NCC(F)(F)F)n2)n3COCC[Si](C)(C)C)cc(-n2cncn2)c1]

N-(3-Methyl-5-(1H-1,2,4-triazol-1-yl)phenyl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine. This reaction was paralleled for two batches: To a solution of 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine (100 mg, 0.22 mmol) and 3-methyl-5-(1H-1,2,4-triazol-1-yl)aniline (57.06 mg, 0.33 mmol) in THF (5 mL) was added sodiumtert-butoxide (2 M, 0.27 mL) and [2-(2-aminophenyl)phenyl]-methylsulfonyloxy-palladium; dicyclohexyl-[3,6-dimethoxy-2-(2,4,6-triisopropylphenyl)phenyl]phosphane (19.79 mg, 0.02 mmol). The mixture was stirred under argon atmosphere at 100° C. for 12 h. LCMS showed 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine was consumed completely and the desired mass was detected. The two batches reaction mixture was concentrated together to give the crude product. The crude product was purified by prep-TLC (Petroleum ether:Ethyl acetate=0:1) to give N-(3-methyl-5-(1H-1,2,4-triazol-1-yl)phenyl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine(240 mg, 0.4 mmol, 92.25% yield) as a yellow solid. MS (ESI): m/z 569.2 [M+1]⁺.

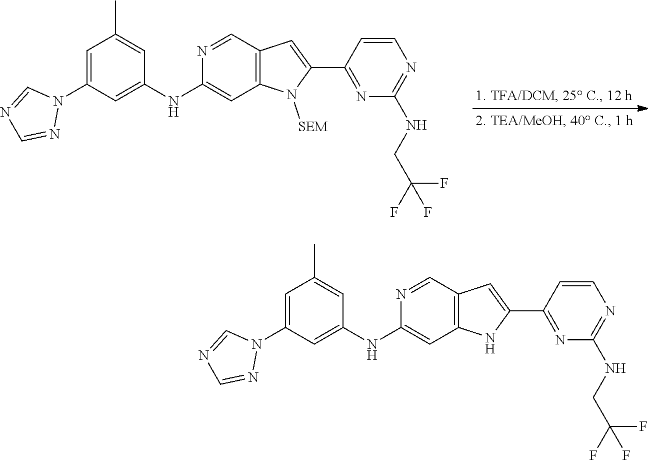 [SMILES: Cc1cc(Nc2cc3c(cn2)cc(-c2ccnc(NCC(F)(F)F)n2)n3COCC[Si](C)(C)C)cc(-n2cncn2)c1 ; Cc1cc(Nc2cc3[nH]c(-c4ccnc(NCC(F)(F)F)n4)cc3cn2)cc(-n2cncn2)c1]

N-(3-Methyl-5-(1H-1,2,4-triazol-1-yl)phenyl)-2-(2-((2,2,2-trifluoroethyl)amino)pyrimidin-4-yl)-1H-pyrrolo[3,2-c]pyridin-6-amine. To a mixture of N-(3-methyl-5-(1H-1,2,4-triazol-1-yl)phenyl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine (240 mg, 0.4 mmol) in dichloromethane (2 mL) and trifluoroacetic acid (2 mL) was stirred at 25° C. for 12 h. LCMS showed N-(3-methyl-5-(1H-1,2,4-triazol-1-yl)phenyl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine was consumed completely. The mixture was concentrated under reduced pressure to remove dichloromethane. To the residue was added methanol (2 mL) and triethylamine (363.50 mg, 3.59 mmol, 0.5 mL). The mixture was stirred at 40° C. for another 1 h. LCMS showed the reaction was complete and the desired mass was detected. The reaction mixture was concentrated to give the crude product. The crude product purified by prep-HPLC (formic acid condition, column: Phenomenex Synergi C18 150*25*10 um; mobile phase: [water(0.2% FA)-ACN]; B %: 10%-35%, 10 min) to give N-(3-methyl-5-(1H-1,2,4-triazol-1-yl)phenyl)-2-(2-((2,2,2-trifluoroethyl)amino)pyrimidin-4-yl)-1H-pyrrolo[3,2-c]pyridin-6-amine (93.24 mg, 0.18 mmol, 44.25% yield, 98% purity, formic acid) as a yellow solid. ¹H NMR (400 MHz, DMSO-d₆) 11.65 (s, 1H), 9.21 (s, 1H), 9.12 (s, 1H), 8.66 (s, 1H), 8.38 (d, J=5.0 Hz, 1H), 8.22 (s, 1H), 8.13 (s, 1H), 7.94 (s, 1H), 7.73 (s, 1H), 7.38 (s, 2H), 7.27 (d, J=5.1 Hz, 1H), 7.18 (s, 1H), 6.98 (s, 1H), 4.50-4.25 (m, 2H), 2.36 (s, 3H). MS (ESI): m/z 466.2 [M+1]⁺.

### Example 13: (R)-2-(2-Methylpyridin-4-yl)-N-(tetrahydrofuran-3-yl)-1H-pyrrolo[3,2-c]pyridin-6-amine

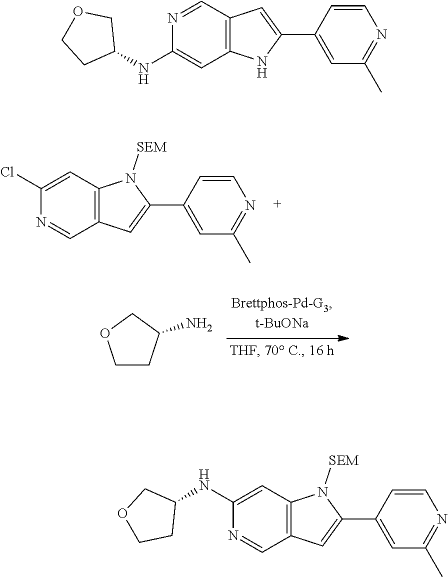 [SMILES: Cc1cc(-c2cc3cnc(N[C@@H]4CCOC4)cc3[nH]2)ccn1 ; Cc1cc(-c2cc3cnc(Cl)cc3n2COCC[Si](C)(C)C)ccn1 ; N[C@@H]1CCOC1 ; Cc1cc(-c2cc3cnc(N[C@@H]4CCOC4)cc3n2COCC[Si](C)(C)C)ccn1]

(R)-2-(2-Methylpyridin-4-yl)-N-(tetrahydrofuran-3-yl)-1-((2-(trimethylsilyl)ethoxy)methyl)-1H-pyrrolo[3,2-c]pyridin-6-amine. To a solution of 6-chloro-2-(2-methylpyridin-4-yl)-1-((2-(trimethylsilyl)ethoxy)methyl)-1H-pyrrolo[3,2-c]pyridine (300 mg, 0.80 mmol) and (R)-tetrahydrofuran-3-amine (209.68 mg, 2.41 mmol) in THF (10 mL) was added Methanesulfonato(2-dicyclohexylphosphino-3,6-dimethoxy-2,4,6-tri-I-propyl-1,1-biphenyl)(2-amino-1,1-biphenyl-2-yl)palladium(II) (72.72 mg, 0.08 mmol) and Sodium tert-Butoxide (1 M, 1.60 mL) under nitrogen, the mixture was stirred at 70° C. for 16 h under nitrogen atmosphere. LCMS indicated 6-chloro-2-(2-methylpyridin-4-yl)-1-((2-(trimethylsilyl)ethoxy)methyl)-1H-pyrrolo[3,2-c]pyridine was consumed and a peak (74%) with desired mass was detected. The mixture was filtered with a pad of Celite, the filtrate was concentrated under vacuum to give a residue. The residue was purified by flash silica gel chromatography (ISCO®; 24 g SepaFlash® Silica Flash Column, Eluent of 0˜100% Ethyl acetate/Petroleum ether gradient, then Ethyl acetate. Methanol=10:1 @ 35 mL/min) to give (R)-2-(2-methylpyridin-4-yl)-N-(tetrahydrofuran-3-yl)-1-((2-(trimethylsilyl)ethoxy)methyl)-1H-pyrrolo[3,2-c]pyridin-6-amine (350 mg, crude) as a yellow solid.

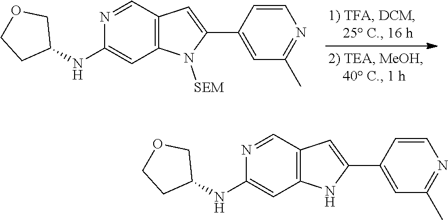 [SMILES: Cc1cc(-c2cc3cnc(N[C@@H]4CCOC4)cc3n2COCC[Si](C)(C)C)ccn1 ; Cc1cc(-c2cc3cnc(N[C@@H]4CCOC4)cc3[nH]2)ccn1]

(R)-2-(2-Methylpyridin-4-yl)-N-(tetrahydrofuran-3-yl)-1H-pyrrolo[3,2-c]pyridin-6-amine. To a solution of (R)-2-(2-methylpyridin-4-yl)-N-(tetrahydrofuran-3-yl)-1-((2-(trimethylsilyl)ethoxy)methyl)-1H-pyrrolo[3,2-c]pyridin-6-amine (350 mg, crude) in dichloromethane (5 mL) was added Trifluoroacetic acid (7.70 g, 67.53 mmol, 5 mL), the mixture was stirred at 25° C. for 16 h. LCMS indicated (R)-2-(2-methylpyridin-4-yl)-N-(tetrahydrofuran-3-yl)-1-((2-(trimethylsilyl)ethoxy)methyl)-1H-pyrrolo[3,2-c]pyridin-6-amine was consumed and a peak (30%) with desired was detected. The mixture was concentrated under vacuum to give a residue. To the residue in methanol (5 mL) was added triethylamine (3.64 g, 35.92 mmol, 5 mL), the mixture was stirred at 40° C. for 1 h. LCMS indicated (R)-2-(2-methylpyridin-4-yl)-N-(tetrahydrofuran-3-yl)-1-((2-(trimethylsilyl)ethoxy)methyl)-1H-pyrrolo[3,2-c]pyridin-6-amine was consumed and a peak (87%) with desired was detected. The mixture was concentrated under reduced pressure to give a residue. The residue was purified by prep-HPLC (column: Unisil 3-100 C18 Ultra 150*50 mm*3 um; mobile phase: [water(0.225% FA)-ACN];B %: 1%-20%,10 min) followed by lyophilization to give (R)-2-(2-methylpyridin-4-yl)-N-(tetrahydrofuran-3-yl)-1H-pyrrolo[3,2-c]pyridin-6-amine (202.86 mg, 557.79 umol,67.67% yield, 93.59% purity, formic acid) as a yellow solid. ¹H NMR (400 MHz, DMSO-d₆) δ 12.53 (s, 1H), 8.67-8.66 (m, 2H), 7.96 (s, 1H), 7.86 (d, J=5.3 Hz, 1H), 7.47 (s, 1H), 6.74 (s, 1H), 4.34-4.33 (m, 1H), 3.92-3.88 (m, 2H), 3.80-3.78 (m, 1H), 3.69-3.68 (m, 1H), 2.61 (s, 3H), 2.32-2.27 (m, 1H), 1.90-1.88 (m, 1H). LCMS (ESI): m/z 295.1 [M+1]⁺.

### Example 14: 2-(2-Methoxypyrimidin-4-yl)-N-(tetrahydro-2H-pyran-4-yl)-1H-pyrrolo[3,2-c]pyridin-6-amine

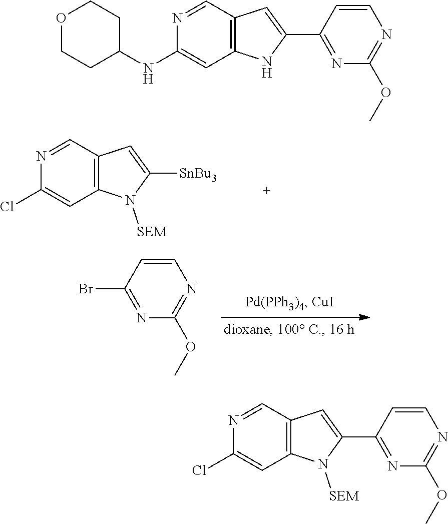

6-Chloro-2-(2-methoxypyrimidin-4-yl)-1-((2-(trimethylsilyl)ethoxy)methyl)-1H-pyrrolo[3,2-c]pyridine. To a solution of 6-chloro-2-(tributylstannyl)-1-((2-(trimethylsilyl)ethoxy)methyl) -1H-pyrrolo[3,2-c]pyridine (1 g, 1.75 mmol) and 4-bromo-2-methoxypyrimidine (379.16 mg, 2.63 mmol) in 1,4-Dioxane (10 mL) were added tetrakis[triphenylphosphine]palladium(0) (202.06 mg, 0.17 mmol) and cuprous iodide (33.30 mg, 0.17 mmol) under nitrogen, the mixture was stirred at 100° C. for 16 h under nitrogen atmosphere. LCMS indicated 6-chloro-2-(tributylstannyl)-1-((2-(trimethylsilyl) ethoxy)methyl)-1H-pyrrolo[3,2-c]pyridine was consumed and a peak with desired mass. The mixture was filtered with a pad of Celite, the filtrate was concentrated under vacuum to give a residue. The residue was purified by flash silica gel chromatography (ISCO® 24 g SepaFlash® Silica Flash Column, Eluent of 0-55% Ethyl acetate/Petroleum ether gradient @ 35 mL/min) to give 6-chloro-2-(2-methoxypyrimidin-4-yl)-1-((2-(trimethylsilyl)ethoxy)methyl)-1H-pyrrolo[3,2-c]pyridine (280 mg, 0.72 mmol, 40.96% yield) as a yellow oil. LCMS (ESI): m/z 391.2 [M+1]⁺.

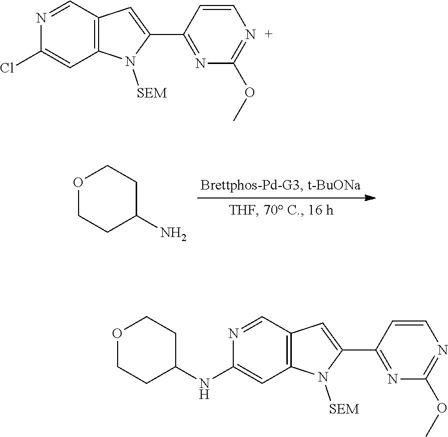

2-(2-Methoxypyrimidin-4-yl)-N-(tetrahydro-2H-pyran-4-yl)-1-((2-(trimethylsilyl)ethoxy)methyl)-1H-pyrrolo[3,2-c]pyridin-6-amine. To a solution of 6-chloro-2-(2-methoxypyrimidin-4-yl)-1-((2-(trimethylsilyl) ethoxy)methyl)-1H-pyrrolo[3,2-c]pyridine (280 mg, 0.72 mmol) and tetrahydropyran-4-amine (217.34 mg, 2.15 mmol) in tetrahydrofuran (10 mL) were added dimethoxy-2,4,6-tri-i-propyl-1,1-biphenyl)(2-amino-1,1-biphenyl-2-yl)palladium(II)(64.93 mg, 0.072 mmol) and Sodium tert-Butoxide (137.66 mg, 1.43 mmol, in THF) under nitrogen, the mixture was stirred at 70° C. for 16 h under nitrogen atmosphere. LCMS indicated 6-chloro-2-(2-methoxypyrimidin-4-yl)-1-((2-(trimethylsilyl)ethoxy)methyl)-1H-pyrrolo[3,2-c]pyridine was consumed and a peak with desired mass was detected. The mixture was filtered with a pad of Celite, the filtrate was concentrated under vacuum to give a residue. The residue was purified by column chromatography (100-200 mesh silica gel, Petroleum ether/Ethyl acetate=5/1, 0/1) to give 2-(2-methoxypyrimidin-4-yl)-N-(tetrahydro-2H-pyran-4-yl)-1-((2-(trimethylsilyl)ethoxy)methyl)-1H-pyrrolo[3,2-c]pyridin-6-amine (230 mg, 0.51 mmol, 70.48% yield) as a yellow oil. LCMS (ESI): m/z 456.3 [M+1]⁺, ¹H NMR (DMSO-d₆, 400 MHz) δ 8.60-8.54 (m, 1H), 8.44 (s, 1H), 7.59 (d, J=5.4 Hz, 1H), 7.39-7.37 (m, 1H), 6.53 (s, 1H), 6.26 (d, J=8.0 Hz, 1H), 6.05-6.03 (m, 2H), 3.95 (s, 3H), 3.90-3.85 (m, 3H), 3.42-3.37 (m, 4H), 1.91-1.88 (m, 2H), 1.49-1.41 (m, 2H), 0.75-0.70 (m, 2H), -0.212 (s, 9H).

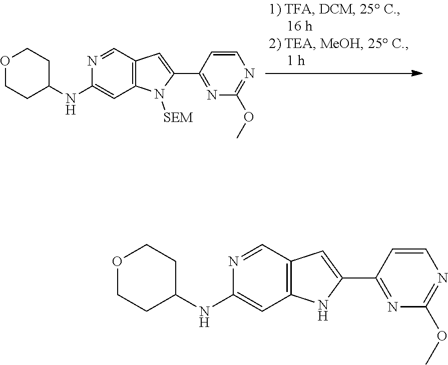 [SMILES: COc1nccc(-c2cc3cnc(NC4CCOCC4)cc3n2COCC[Si](C)(C)C)n1 ; COc1nccc(-c2cc3cnc(NC4CCOCC4)cc3[nH]2)n1]

2-(2-Methoxypyrimidin-4-yl)-N-(tetrahydro-2H-pyran-4-yl)-1H-pyrrolo[3,2-c]pyridin-6-amine. To a solution of 2-(2-methoxypyrimidin-4-yl)-N-tetrahydropyran-4-yl-1-(2-trimethylsilylethoxymethyl)pyrrolo[3,2-c]pyridin-6-amine (230 mg, 0.50 mmol) in dichloromethane (5 mL) was added Trifluoroacetic acid (5. mL, 0.5000 mmol), the mixture was stirred at 25° C. for 16 h. LCMS indicated 2-(2-methoxypyrimidin-4-yl)-N-tetrahydropyran-4-yl-1-(2-trimethylsilylethoxymethyl)pyrrolo[3,2-c]pyridin-6-amine was consumed and a peak with desired mass of intermediate. The mixture was concentrated under vacuum to give a residue. To the residue in Methanol (5 mL) was added triethylamine (5. mL, 0.5000 mmol), the mixture was stirred at 25° C. for 1 h. LCMS indicated 2-(2-methoxypyrimidin-4-yl)-N-tetrahydropyran-4-yl-1-(2-trimethylsilylethoxymethyl)pyrrolo[3,2-c]pyridin-6-amine was consumed and a peak with desired mass. The mixture was concentrated under reduced pressure to give a residue. The residue was purified by prep-HPLC (column: Unisil 3-100 C18 Ultra 150*50 mm*3 um; mobile phase: [water(0.225% FA)-ACN];B %: 10%-30%, 100 min) and dried by lyophilization to give 2-(2-methoxypyrimidin-4-yl)-N-(tetrahydro-2H-pyran-4-yl)-1H-pyrrolo[3,2-c]pyridin-6-amine (62.27 mg, 0.19 mmol, 37.76% yield, formic acid) as a yellow solid. LCMS (ESI): m/z 326.3 [M+1]⁺, ¹H NMR (DMSO-d₆, 400 MHz) δ 12.03 (s, 1H), 8.65 (d, J=5.3 Hz, 1H), 8.59 (s, 1H), 8.14 (s, 0.27H), 7.70 (d, J=5.3 Hz, 1H), 7.49 (d, J=1.0 Hz, 1H), 7.32 (s, 1H), 6.71 (s, 1H), 4.02 (s, 3H), 3.91-3.90 (m, 2H), 3.80-3.89 (m, 1H), 3.46-3.36 (m, 2H), 1.95-1.91 (m, 2H), 1.55-1.47 (m, 2H).

### Example 15: 1-Methyl-2-(2-methylpyridin-4-yl)-N-(tetrahydro-2H-pyran-4-yl)-1H-pyrrolo[3,2-c]pyridin-6-amine

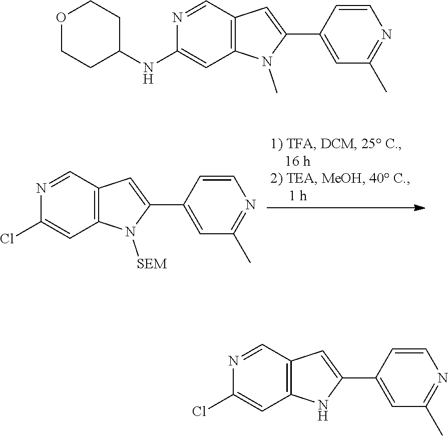 [SMILES: Cc1cc(-c2cc3cnc(NC4CCOCC4)cc3n2C)ccn1 ; Cc1cc(-c2cc3cnc(Cl)cc3n2COCC[Si](C)(C)C)ccn1 ; Cc1cc(-c2cc3cnc(Cl)cc3[nH]2)ccn1]

6-Chloro-2-(2-methylpyridin-4-yl)-1H-pyrrolo[3,2-c]pyridine. To a solution of 6-chloro-2-(2-methylpyridin-4-yl)-1-((2-(trimethylsilyl)ethoxy)methyl)-1H-pyrrolo[3,2-c]pyridine (1. g, 2.67 mmol) in dichloromethane (10 mL) was added trifluoroacetic acid (10. mL, 2.67 mmol). The reaction mixture was stirred at 25° C. for 12 h. LCMS showed a peak (83.2%) with desired mass and a peak (8.2%) with mass of (6-chloro-2-(2-methylpyridin-4-yl)-1H-pyrrolo[3,2-c]pyridin-1-yl)methanol. The reaction mixture was concentrated under reduced pressure to give a residue. To the residue in methanol (5 mL) was added triethylamine (5 mL, 35.87 mmol), the mixture was stirred at 40° C. for 1 h, LCMS showed (6-chloro-2-(2-methylpyridin -4-yl)-1H-pyrrolo[3,2-c]pyridin-1-yl)methanol was consumed completely and a main peak with desired mass. The mixture was concentrated in reduced pressure. The residue was poured into water (40 mL). The aqueous phase was extracted with ethyl acetate (40 mL×3). The combined organic phase was washed with brine (40 mL×2), dried over anhydrous sodium sulfate, filtered and concentrated under vacuum. The residue was purified by silica gel chromatography (200-300 mesh silica gel, Petroleum ether/Ethyl acetate=100/1, 2/1, Rf=0.5, Petroleum ether/Ethyl acetate=1/1) to give 6-chloro-2-(2-methylpyridin-4-yl)-1H-pyrrolo[3,2-c]pyridine (500 mg, 1.982 mmol, 74.11% yield) as a yellow solid.

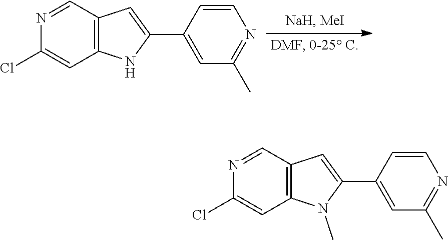 [SMILES: Cc1cc(-c2cc3cnc(Cl)cc3[nH]2)ccn1 ; Cc1cc(-c2cc3cnc(Cl)cc3n2C)ccn1]

6-Chloro-1-methyl-2-(2-methylpyridin-4-yl)-1H-pyrrolo[3,2-c]pyridine. To a solution of 6-chloro-2-(2-methylpyridin-4-yl)-1H-pyrrolo[3,2-c]pyridine (450. mg, 1.85 mmol) in DMF (30 mL) was added hydrogen sodium (147.73 mg, 3.69 mmol, 60% purity) at 0° C., the reaction mixture was stirred at 0° C. for 0.5 h. After that, the reaction mixture was added iodomethane (1.3 g, 9.16 mmol), the reaction mixture was stirred at 0° C. for another 1 h, and then reacted for 2 h at 25° C. LCMS showed 6-chloro-2-(2-methyl-4-pyridyl)-1H-pyrrolo[3,2-c]pyridine was consumed completely and a peak (97%) with desired mass. The mixture was poured into water (40 mL). The aqueous phase was extracted with ethyl acetate (40 mL×3). The combined organic phase was washed with brine (40 mL×2), dried over anhydrous sodium sulfate, filtered and concentrated under vacuum. The residue was purified by silica gel chromatography (200-300 mesh silica gel, Petroleum ether/Ethyl acetate=100/1, 1/1) to give 6-chloro-1-methyl-2-(2-methylpyridin-4-yl)-1H-pyrrolo[3,2-c]pyridine (300 mg, 1.1547 mmol, 62.533% yield) as a yellow solid.

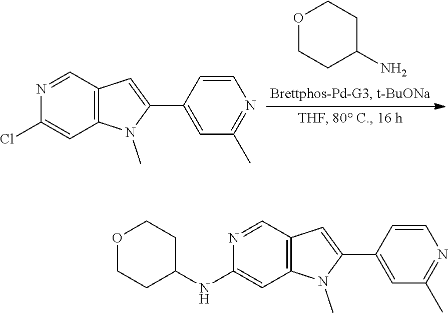 [SMILES: NC1CCOCC1 ; Cc1cc(-c2cc3cnc(Cl)cc3n2C)ccn1 ; Cc1cc(-c2cc3cnc(NC4CCOCC4)cc3n2C)ccn1]

1-Methyl-2-(2-methylpyridin-4-yl)-N-(tetrahydro-2H-pyran-4-yl)-1H-pyrrolo[3,2-c]pyridin-6-amine. To a solution of 6-chloro-1-methyl-2-(2-methylpyridin-4-yl)-1H-pyrrolo[3,2-c]pyridine (150. mg, 0.5800 mmol) and tetrahydro-2H-pyran-4-amine (176.62 mg, 1.75 mmol) in 1,4-Dioxane (4 mL) was added sodium tert-butoxide (1.16 mL, 1.16 mmol, 1 M in THF) and methanesulfonato(2-dicyclohexylphosphino-3,6-dimethoxy-2′,4′,6′-tri-i-propyl-1,1′-biphenyl)(2′-amino-1,1′-biphenyl-2-yl)palladium (II) (105.52 mg, 0.1200 mmol). The reaction mixture was stirred at 80° C. and reacted for 12 h under nitrogen. LCMS showed 6-chloro-1-methyl-2-(2-methylpyridin-4-yl)-1H-pyrrolo[3,2-c]pyridine was consumed completely and a peak (65.5%) with desired mass. The mixture was cooled and concentrated in reduced pressure. The residue was poured into water (40 mL). The aqueous phase was extracted with ethyl acetate (40 mL×3). The combined organic phase was washed with brine (40 mL×2), dried over anhydrous sodium sulfate, filtered and concentrated under vacuum. The residue was purified by prep-HPLC (Phenomenex Synergi C18 150*25*10 um; mobile phase: [water(0.225% FA)-ACN]; B %: 0%-15%, 10 min) followed by lyophilization to give 1-methyl-2-(2-methylpyridin-4-yl)-N-(tetrahydro-2H-pyran-4-yl)-1H-pyrrolo[3,2-c]pyridin-6-amine (96.26 mg, 0.2983 mmol, 51.248% yield, formic acid) as a yellow solid. Additional 2.34 mg for delivery, ¹H NMR (400 MHz, DMSO-d₆) δ=8.51 (d, J=5.3 Hz, 1H), 8.36 (s, 1H), 8.17 (s, 1H), 7.45 (s, 1H), 7.38 (dd, J₁=5.1, J₂=1.3 Hz, 1H), 6.68 (s, 1H), 6.41 (s, 1H), 6.07-5.88 (m, 1H), 3.91-3.85 (m, 3H), 3.65 (s, 3H), 3.47-3.41 (m, 2H), 2.54 (s, 3H), 1.93-1.86 (m, 2H), 1.51-1.37 (m, 2H). LCMS (ESI): m/z 323.1 [M+1]⁺.

### Example 16: 2-(2-Methylpyrimidin-4-yl)-N-(tetrahydro-2H-pyran-4-yl)-1H-pyrrolo[3,2-c]pyridin-6-amine

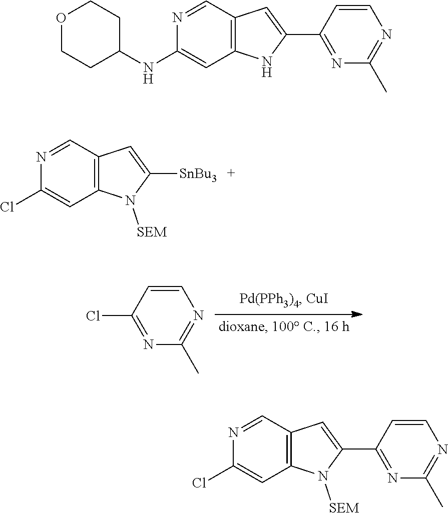

6-Chloro-2-(2-methylpyrimidin-4-yl)-1-((2-(trimethylsilyl)ethoxy)methyl)-1H-pyrrolo[3,2-c]pyridine. To a solution of 6-chloro-2-(tributylstannyl)-1-((2-(trimethylsilyl)ethoxy)methyl) -1H-pyrrolo[3,2-c]pyridine (1 g, 1.75 mmol) and 4-chloro-2-methyl-pyrimidine (337.2 mg, 2.63 mmol) in 1,4-Dioxane (10 mL) were added tetrakis[triphenylphosphine]palladium(0) (202.06 mg, 0.17 mmol) and cuprous iodide (33.30 mg, 0.17 mmol) under nitrogen, the mixture was stirred at 100° C. for 16 h under nitrogen atmosphere. LCMS indicated 6-chloro-2-(tributylstannyl)-1-((2-(trimethylsilyl)ethoxy)methyl)-1H-pyrrolo[3,2-c]pyridine was consumed and a peak with desired mass was detected. The mixture was filtered with a pad of Celite, the filtrate was concentrated under vacuum to give a residue. The residue was purified by flash silica gel chromatography (ISCO®; 24 g SepaFlash® Silica Flash Column, Eluent of 0˜55% Ethyl acetate/Petroleum ether gradient @ 35 mL/min) to give 6-chloro-2-(2-methylpyrimidin-4-yl)-1-((2-(trimethylsilyl)ethoxy)methyl)-1H-pyrrolo[3,2-c]pyridine (240 mg, 0.64 mmol, 36.61% yield) as a yellow oil. LCMS (ESI): m/z 375.2 [M+1]⁺.

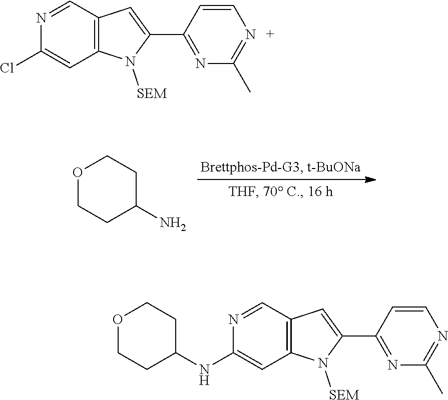

2-(2-Methylpyrimidin-4-yl)-N-(tetrahydro-2H-pyran-4-yl)-1-((2-(trimethylsilyl)ethoxy)methyl)-1H-pyrrolo[3,2-c]pyridin-6-amine. To a solution of 6-chloro-2-(2-methylpyrimidin-4-yl)-1-((2-(trimethylsilyl)ethoxy)methyl)-1H-pyrrolo[3,2-c]pyridine (240. mg, 0.64 mmol) and tetrahydropyran-4-amine (194.24 mg, 1.92 mmol) in tetrahydrofuran (5 mL) were added Sodium tert-Butoxide (1.28 mL, 1.28 mmol, 1M in THF) and Methanesulfonato(2-dicyclohexylphosphino-3,6-dimethoxy-2,4,6-tri-i-propyl-1,1-biphenyl)(2-amino-1,1-biphenyl-2-yl)palladium(II) (58.03 mg, 0.06 mmol) under nitrogen, the mixture was stirred at 70° C. for 16 h under nitrogen atmosphere. LCMS indicated 6-chloro-2-(2-methylpyrimidin-4-yl)-1-((2-(trimethylsilyl)ethoxy)methyl)-1H-pyrrolo[3,2-c]pyridine was consumed and a peak with desired mass was detected. The mixture was concentrated under reduced pressure to give a residue. The residue was purified by flash silica gel chromatography (ISCO®; 24 g SepaFlash® Silica Flash Column, Eluent of 0˜100% Ethyl acetate/Petroleum ether radient @ 35 mL/min) to give 2-(2-methylpyrimidin-4-yl)-N-(tetrahydro-2H-pyran-4-yl)-1-((2-(trimethylsilyl)ethoxy)methyl)-1H-pyrrolo[3,2-c]pyridin-6-amine (230 mg, 0.52 mmol, 81.73% yield) was obtained as a yellow solid. LCMS (ESI): m/z 440.3 [M+1]⁺.

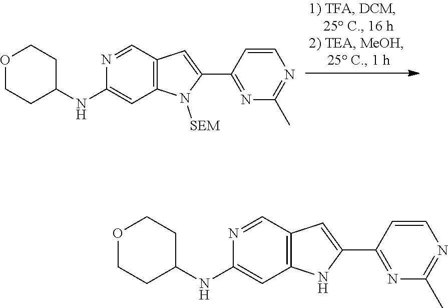 [SMILES: Cc1nccc(-c2cc3cnc(NC4CCOCC4)cc3n2COCC[Si](C)(C)C)n1 ; Cc1nccc(-c2cc3cnc(NC4CCOCC4)cc3[nH]2)n1]

2-(2-Methylpyrimidin-4-yl)-N-(tetrahydro-2H-pyran-4-yl)-1H-pyrrolo[3,2-c]pyridin-6-amine. To a solution of 2-(2-methylpyrimidin-4-yl)-N-(tetrahydro-2H-pyran-4-yl)-1-((2-(trimethylsilyl)ethoxy)methyl)-1H-pyrrolo[3,2-c]pyridin-6-amine (230. mg, 0.52 mmol) in dichloromethane (5 mL)was added Trifluoroacetic acid (5. mL, 0.52 mmol), the mixture was stirred at 25° C. for 16 h. LCMS indicated 2-(2-methylpyrimidin-4-yl)-N-(tetrahydro-2H-pyran-4-yl)-1-((2-(trimethylsilyl)ethoxy)methyl)-1H-pyrrolo[3,2-c]pyridin-6-amine was consumed and a peak with the mass of intermediate. The mixture was concentrated under vacuum to give a residue. The residue in Methanol (5 mL) was added triethylamine (5 mL, 0.50 mmol), the mixture was stirred at 25° C. for 1 h. LCMS indicated a main peak with desired mass. The mixture was concentrated under reduced pressure to give a residue. The residue was purified by prep-HPLC(column: Unisil 3-100 C18 Ultra 150*50 mm*3 um; mobile phase: [water(0.225% FA)-ACN];B %: 3%-33%,10 min) then dried by lyophilization to give 2-(2-methylpyrimidin-4-yl)-N-(tetrahydro-2H-pyran-4-yl)-1H-pyrrolo[3,2-c]pyridin-6-amine (2.11 mg, 0.0066 mmol, 1.27% yield) and 2-(2-methylpyrimidin-4-yl)-N-(tetrahydro-2H-pyran-4-yl)-1H-pyrrolo[3,2-c]pyridin-6-amine (117.53 mg, 0.37 mmol, 70.51% yield, formic acid) as a yellow solid. LCMS (ESI): m/z 310.2 [M+1]⁺. ¹H NMR (400 MHz, DMSO-d₆) δ 11.86 (s, 1H), 8.72 (d, J=5.4 Hz, 1H), 8.54 (s, 1H), 7.84 (d, J=5.3 Hz, 1H), 7.43 (s, 1H), 7.04 (s, 1H), 6.66 (s, 1H), 3.91-3.89 (m, 2H), 3.88-3.87 (m, 1H), 3.46-3.40 (m, 2H), 2.69 (s, 3H), 1.93-1.90 (m, 2H), 1.50-1.46 (m, 2H).

### Example 17: 2-(2-Methylpyridin-4-yl)-N-(4-methyltetrahydro-2H-pyran-4-yl)-1H-pyrrolo[3,2-c]pyridin-6-amine

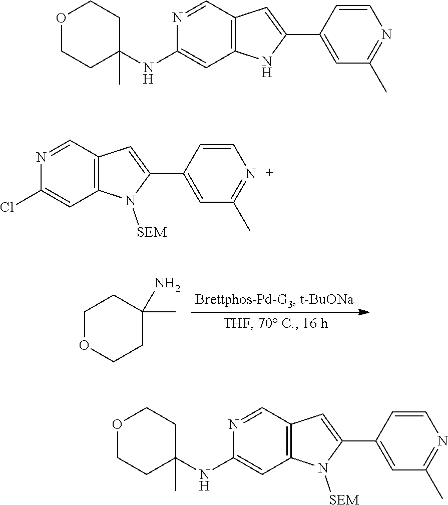

2-(2-Methylpyridin-4-yl)-N-(4-methyltetrahydro-2H-pyran-4-yl)-1H-pyrrolo[3,2-c]pyridin-6-amine. To a mixture of 6-chloro-2-(2-methylpyridin-4-yl)-1-((2-(trimethylsilyl)ethoxy)methyl)-1H-pyrrolo[3,2-c]pyridine (300 mg, 0.80 mmol) and 4-methyltetrahydro-2H-pyran-4-amine (277.18 mg, 2.41 mmol) in THF (5 mL) were added methanesulfonato(2-dicyclohexylphosphino-3,6-dimethoxy -2,4,6-tri-I-propyl-1,1-biphenyl)(2-amino-1,1-biphenyl-2-yl)palladium(II) (72.72 mg, 0.08 mmol) and sodium tert-butoxide (1.6 mL, 1.6 mmol, 1 M in THF). The mixture was stirred at 70° C. for 16 h under nitrogen atmosphere. LCMS showed 6-chloro-2-(2-methylpyridin-4-yl)-1-((2-(trimethylsilyl)ethoxy)methyl)-1H-pyrrolo[3,2-c]pyridine was consumed completely and a peak (35%) with desired mass. The mixture was concentrated under reduced pressure to give a residue. The residue was purified by flash silica gel chromatography (Biotage, 24 g SepaFlash® Silica Flash Column, Eluent of 0˜100% Ethyl acetate/Petroleum ether gradient @ 35 mL/min, TLC (Petroleum ether:Ethyl acetate=0:1) Rf SM=0.7 Rf DP=0.4) to give 2-(2-methylpyridin-4-yl)-N-(4-methyltetrahydro-2H-pyran-4-yl)-1-((2-(trimethylsilyl)ethoxy)methyl)-1H-pyrrolo[3,2-c]pyridin-6-amine (110 mg, 0.24 mmol, 30.29% yield) was obtained as a yellow oil. MS (ESI): m/z 453.0 [M+1]⁺; ¹H NMR (400 MHz, DMSO-d₆) δ=8.50 (d, J=5.1 Hz, 1H), 8.35 (s, 1H), 7.51 (s, 1H), 7.45-7.44 (m, 1H), 6.77 (s, 1H), 6.67 (s, 1H), 5.80 (s, 1H), 5.37 (s, 2H), 3.65-3.56 (m, 4H), 3.51-3.47 (m, 2H), 2.52 (s, 3H), 2.23-2.19 (m, 2H), 1.66-1.59 (m, 2H), 1.43 (s, 3H), 0.86-0.82 (m, 2H), −0.06-0.09 (m, 9H).

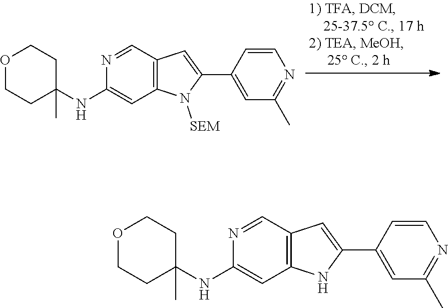 [SMILES: Cc1cc(-c2cc3cnc(NC4(C)CCOCC4)cc3n2COCC[Si](C)(C)C)ccn1 ; Cc1cc(-c2cc3cnc(NC4(C)CCOCC4)cc3[nH]2)ccn1]

2-(2-Methylpyridin-4-yl)-N-(4-methyltetrahydro-2H-pyran-4-yl)-1H-pyrrolo[3,2-c]pyridin-6-amine. To a mixture of 2-(2-methylpyridin-4-yl)-N-(4-methyltetrahydro-2H-pyran-4-yl)-1-((2-(trimethylsilyl)ethoxy)methyl)-1H-pyrrolo[3,2-c]pyridin-6-amine (110. mg, 0.24 mmol) in dichloromethane (2 mL) was added Trifluoroacetic acid (2. mL, 0.24 mmol). The mixture was stirred at 25° C. for 16 h. LCMS showed 2-(2-methylpyridin-4-yl)-N-(4-methyltetrahydro-2H-pyran-4-yl)-1-((2-(trimethylsilyl)ethoxy)methyl)-1H-pyrrolo[3,2-c]pyridin-6-amine remained (5%) and a peak (25%) with desired mass. The mixture was stirred at 37.5° C. for 1 h. TLC (Dichloromethane:Methanol=10:1) showed 2-(2-methylpyridin-4-yl)-N-(4-methyltetrahydro-2H-pyran-4-yl)-1-((2-(trimethylsilyl)ethoxy)methyl)-1H-pyrrolo[3,2-c]pyridin-6-amine was consumed and a new peak (Rf=0.3). The mixture was concentrated under reduced pressure to give a residue. To the residue in Methanol (2 mL) was added triethylamine (2. mL, 0.2400 mmol). The mixture was stirred at 25° C. for 2 h. LCMS showed 2-(2-methylpyridin-4-yl)-N-(4-methyltetrahydro-2H-pyran-4-yl)-1-((2-(trimethylsilyl)ethoxy)methyl)-1H-pyrrolo[3,2-c]pyridin-6-amine was consumed completely and a peak (88%) with desired mass. The mixture was concentrated under reduced pressure to give a residue, which was purified by prep-HPLC (column: Unisil 3-100 C18 Ultra 150*50 mm*3 um; mobile phase: [water (0.225% FA)-ACN]; B %: 1%-20%, 10 min) followed by lyophilization to give 2-(2-methylpyridin-4-yl)-N-(4-methyltetrahydro -2H-pyran-4-yl)-1H-pyrrolo[3,2-c]pyridin-6-amine (43.42 mg, 0.134 mmol, 54.92% yield, formic acid) as a yellow solid. MS (ESI): m/z 323.1 [M+1]⁺; ¹H NMR (400 MHz, DMSO-d₆) δ 12.54 (s, 1H), 8.65 (m, 1H), 8.64 (d, J=5.6 Hz, 1H), 7.92 (s, 1H), 7.83 (d, J=5.3 Hz, 1H), 7.46 (s, 1H), 7.08-7.01 (m, 1H), 6.82 (s, 1H), 3.65-3.63 (m, 4H), 2.60 (s, 3H), 2.04-2.00 (m 2H), 1.77-1.70 (m, 2H), 1.47 (s, 3H).

### Example 18: (S)-2-(2-Methylpyridin-4-yl)-N-(tetrahydrofuran-3-yl)-1-((2-(trimethylsilyl)ethoxy)methyl)-1H-pyrrolo[3,2-c]pyridin-6-amine

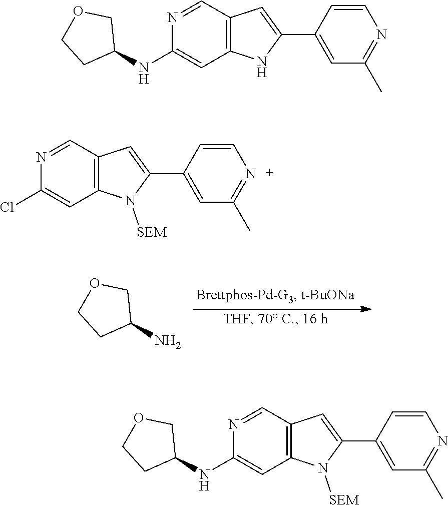 [SMILES: Cc1cc(-c2cc3cnc(N[C@H]4CCOC4)cc3[nH]2)ccn1 ; Cc1cc(-c2cc3cnc(Cl)cc3n2COCC[Si](C)(C)C)ccn1 ; N[C@H]1CCOC1 ; Cc1cc(-c2cc3cnc(N[C@H]4CCOC4)cc3n2COCC[Si](C)(C)C)ccn1]

(S)-2-(2-Methylpyridin-4-yl)-N-(tetrahydrofuran-3-yl)-1-((2-(trimethylsilyl)ethoxy)methyl)-1H-pyrrolo[3,2-c]pyridin-6-amine. To a mixture of 6-chloro-2-(2-methylpyridin-4-yl)-1-((2-(trimethylsilyl)ethoxy)methyl)-1H-pyrrolo[3,2-c]pyridine (300 mg, 0.80 mmol) and (S)-tetrahydrofuran-3-amine (209.68 mg, 2.41 mmol) in THF (5 mL) was added methanesulfonato(2-dicyclohexylphosphino-3,6-dimethoxy-2,4,6-tri-I-propyl-1,1-biphenyl)(2-amino-1,1-biphenyl-2-yl)palladium(II) (72.72 mg, 0.08 mmol) and sodium tert-butoxide (1.6 mL, 1.6 mmol, 1M in THF). The mixture was stirred at 70° C. for 16 h under nitrogen atmosphere. LCMS 6-chloro-2-(2-methylpyridin-4-yl) -1-((2-(trimethylsilyl)ethoxy)methyl)-1H-pyrrolo[3,2-c]pyridine was consumed completely and a peak (77%) with desired mass. The mixture was concentrated under reduced pressure to give a residue. The residue was purified by flash silica gel chromatography (Biotage, 24 g SepaFlash® Silica Flash Column, Eluent of 0˜100% Ethyl acetate/Petroleum ether, Ethyl acetate:Methanol=10:1 gradient @ 35 mL/min). TLC (Ethyl acetate:Methanol=10:1) Rf SM=0.8 Rf DP=0.4) to give (S)-2-(2-methylpyridin-4-yl)-N-(tetrahydrofuran-3-yl)-1-((2-(trimethylsilyl)ethoxy)methyl)-1H-pyrrolo[3,2-c]pyridin-6-amine (330 mg, 0.78 mmol, 96.88% yield) as a yellow oil. MS (ESI): m/z 425.0 [M+1]⁺.

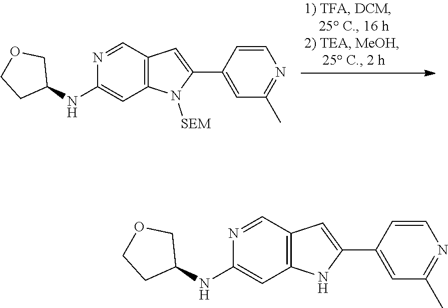 [SMILES: Cc1cc(-c2cc3cnc(N[C@H]4CCOC4)cc3n2COCC[Si](C)(C)C)ccn1 ; Cc1cc(-c2cc3cnc(N[C@H]4CCOC4)cc3[nH]2)ccn1]

(S)-2-(2-Methylpyridin-4-yl)-N-(tetrahydrofuran-3-yl)-1H-pyrrolo[3,2-c]pyridin-6-amine. To a mixture of (S)-2-(2-methylpyridin-4-yl)-N-(tetrahydrofuran-3-yl)-1-((2-(trimethylsilyl)ethoxy)methyl)-1H-pyrrolo[3,2-c]pyridin-6-amine (330. mg, 0.78 mmol) in dichloromethane (5 mL) was added trifluoroacetic acid (5. mL, 0.78 mmol). The mixture was stirred at 25° C. for 16 h. LCMS showed (S)-2-(2-methylpyridin-4-yl)-N-(tetrahydrofuran-3-yl)-1-((2-(trimethylsilyl)ethoxy)methyl)-1H-pyrrolo[3,2-c]pyridin-6-amine remained and a peak (23%) with desired mass. The mixture was concentrated under reduced pressure to give a residue. The residue in Methanol (5 mL) was added triethylamine (5. mL, 0.78 mmol) was stirred at 25° C. for 2 h. LCMS showed (S)-2-(2-methylpyridin-4-yl)-N-(tetrahydrofuran-3-yl)-1-((2-(trimethylsilyl)ethoxy)methyl)-1H-pyrrolo[3,2-c]pyridin-6-amine was consumed completely and a peak (94%) with desired mass. The mixture was concentrated under reduced pressure to give a residue. The residue was purified by prep-HPLC (column: Unisil 3-100 C18 Ultra 150*50 mm*3 um; mobile phase: [water (0.225% FA)-ACN]; B %: 1%-20%, 10 min) followed by lyophilization to give (S)-2-(2-methylpyridin-4-yl)-N-(tetrahydrofuran-3-yl)-1H-pyrrolo[3,2-c]pyridine-6-amine (207.36 mg, 0.69 mmol, 88.83% yield, formic acid) as a yellow solid. MS (ESI): m/z 295.1 [M+1]⁺; ¹H NMR (400 MHz, DMSO-d₆) δ 12.50 (s, 1H), 8.68 (d, J=5.7 Hz, 1H), 8.65 (s, 1H), 7.95 (s, 1H), 7.86 (d, J=5.1 Hz, 1H), 7.80 (d, J=2.6 Hz, 1H), 7.47 (s, 1H), 6.74 (s, 1H), 4.34-4.33 (m, 1H), 3.91-3.88 (m, 2H), 3.80-3.78 (m, 1H), 3.69-3.68 (m, 1H), 2.62 (s, 3H), 2.33-2.29 (m, 1H), 1.89-1.88 (m, 1H).

### Example 19: 2-(Oxazol-5-yl)-N-(tetrahydro-2H-pyran-4-yl)-1H-pyrrolo[3,2-c]pyridin-6-amine

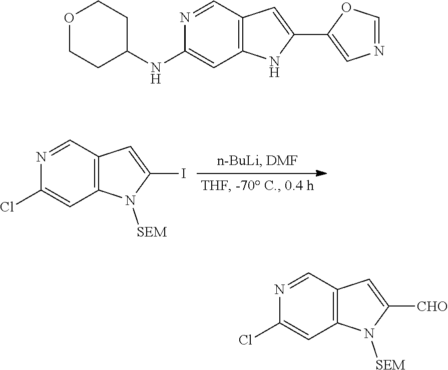 [SMILES: c1ncc(-c2cc3cnc(NC4CCOCC4)cc3[nH]2)o1 ; C[Si](C)(C)CCOCn1c(I)cc2cnc(Cl)cc21 ; C[Si](C)(C)CCOCn1c(C=O)cc2cnc(Cl)cc21]

6-Chloro-1-((2-(trimethylsilyl)ethoxy)methyl)-1H-pyrrolo[3,2-c]pyridine-2-carbaldehyde. To a solution of 6-chloro-2-iodo-1-((2-(trimethylsilyl)ethoxy)methyl)-1H-pyrrolo[3,2-c]pyridine (2. g, 4.89 mmol) in THF(20 mL) was added Butyllithium (2.94 mL, 7.34 mmol, 2.5 M) dropwise at −70° C. The mixture was stirred at −70° C. for 10 min. Then DMF (1.07 g, 14.68 mmol) was added. The mixture stirred at −70° C. for 30 min. TLC (Petroleum ether:Ethyl acetate=3:1) showed 2-[(6-chloro-2-iodo-pyrrolo[3,2-c]pyridin-1-yl) methoxy]ethyl-trimethylsilane was consumed and one main spot with lower polarity The mixture was poured into water (50 mL). The aqueous phase was extracted with ethyl acetate (50 mL×3). The combined organic phase was washed with brine (30 mL×2), dried over anhydrous sodium sulfate, filtered and concentrated under vacuum. The residue was purified by flash silica gel chromatography (Biotage, 40 g SepaFlash® Silica Flash Column, Eluent of 0-20% Ethyl acetate/Petroleum ether gradient @ 40 mL/min) to give 6-chloro-1-((2-(trimethylsilyl)ethoxy)methyl)-1H-pyrrolo[3,2-c]pyridine-2-carbaldehyde (1000 mg, 3.217 mmol, 65.746% yield) as a white solid. MS (ESI): m/z 311.0 [M+1]⁺.

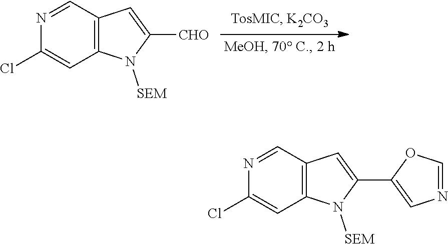 [SMILES: C[Si](C)(C)CCOCn1c(C=O)cc2cnc(Cl)cc21 ; C[Si](C)(C)CCOCn1c(-c2cnco2)cc2cnc(Cl)cc21]

5-(6-Chloro-1-((2-(trimethylsilyl)ethoxy)methyl)-1H-pyrrolo[3,2-c]pyridin-2-yl)oxazole. To a solution of 6-chloro-1-((2-(trimethylsilyl)ethoxy)methyl)-1H-pyrrolo[3,2-c]pyridine-2-carbaldehyde (970. mg, 3.12 mmol) and 1-(isocyanomethylsulfonyl)-4-methyl-benzene (913.86 mg, 4.68 mmol) in Methanol (20 mL) was added potassium carbonate (862.56 mg, 6.24 mmol). The mixture was stirred at 70° C. for 2 h. LCMS showed 6-chloro-1-((2-(trimethylsilyl)ethoxy)methyl)-1H-pyrrolo[3,2-c]pyridine-2-carbaldehyde was consumed completely and a peak (82%) with desired mass. The mixture was concentrated under reduced pressure to give a residue. The residue was purified by flash silica gel chromatography (Biotage, 24 g SepaFlash® Silica Flash Column, Eluent of 0˜37% Ethyl acetate/Petroleum ether gradient @ 35 mL/min) to give 5-(6-chloro-1-((2-(trimethylsilyl)ethoxy)methyl)-1H-pyrrolo[3,2-c]pyridin-2-yl)oxazole (1.05 g, 3.00 mmol, 96.17% yield) as a white solid. ¹H NMR (400 MHz, DMSO-d₆) δ 8.71 (s, 1H), 8.61 (s, 1H), 7.96 (s, 1H), 7.68 (s, 1H), 7.12 (s, 1H), 5.76 (s, 2H), 3.51-3.47 (t, J=7.9 Hz, 2H), 0.81-0.76 (m, 2H), -0.14-0.16 (m, 9H). LCMS (ESI): m/z 350.0 [M+1]⁺.

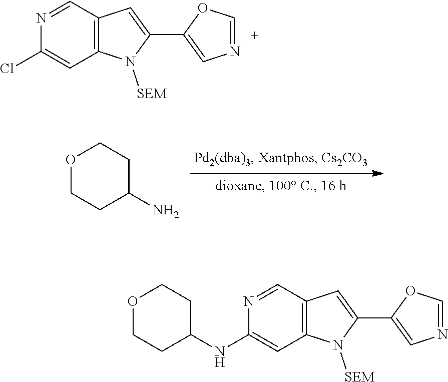

2-(Oxazol-5-yl)-N-(tetrahydro-2H-pyran-4-yl)-1-((2-(trimethylsilyl)ethoxy)methyl)-1H-pyrrolo[3,2-c]pyridin-6-amine. To a mixture of 5-(6-chloro-1-((2-(trimethylsilyl)ethoxy)methyl)-1H-pyrrolo[3,2-c]pyridin-2-yl)oxazole (0.6 g, 1.71 mmol) and tetrahydropyran-4-amine (346.91 mg, 3.43 mmol) in 1,4-Dioxane (5 mL) was added (5-diphenylphosphinyl-9,9-dimethylxanthen-4-yl)-diphenylphosphine (198.45 mg, 0.34 mmol), tris(dibenzylideneacetone)dipalladium(0) (314.06 mg, 0.34 mmol) and cesium carbonate (1676.17 mg, 5.14 mmol). The mixture was stirred at 100° C. for 16 h under nitrogen atmosphere. LCMS showed 5-(6-chloro-1-((2-(trimethylsilyl)ethoxy)methyl)-1H-pyrrolo[3,2-c]pyridin-2-yl)oxazole was consumed completely and a peak with desired mass. The mixture was concentrated under reduced pressure to give a residue. The residue was purified by flash silica gel chromatography (Biotage, 24 g SepaFlash® Silica Flash Column, Eluent of 0˜80% Ethyl acetate/Petroleum ether gradient @ 30 mL/min) to give 2-(oxazol-5-yl)-N-(tetrahydro-2H-pyran-4-yl)-1-((2-(trimethylsilyl)ethoxy)methyl)-1H-pyrrolo[3,2-c]pyridin-6-amine (80 mg, 0.19 mmol, 11.25% yield) as a yellow oil. MS (ESI): m/z 415.1 [M+1]⁺.

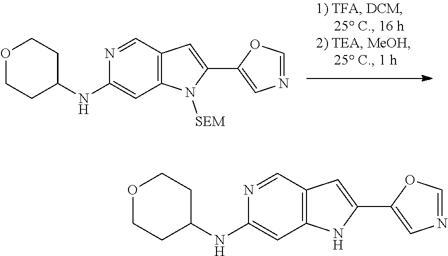 [SMILES: C[Si](C)(C)CCOCn1c(-c2cnco2)cc2cnc(NC3CCOCC3)cc21 ; c1ncc(-c2cc3cnc(NC4CCOCC4)cc3[nH]2)o1]

N-(2-(Trifluoromethyl)-1H-imidazo[4,5-c]pyridin-6-yl)cyclopropanecarboxamide. To a mixture of 2-(oxazol-5-yl)-N-(tetrahydro-2H-pyran-4-yl)-1-((2-(trimethylsilyl) ethoxy)methyl)-1H-pyrrolo[3,2-c]pyridin-6-amine (160. mg, 0.3900 mmol) in dichloromethane (2 mL) was added Trifluoroacetic acid (2. mL, 0.3900 mmol). The mixture was stirred at 25° C. for 16 h. TLC (Petroleum ether:Methanol=10:1) indicated starting material was consumed and two new spot. The mixture was concentrated under reduced pressure to give a residue. Then to the residue in Methanol (2 mL) was added triethylamine (2. mL, 0.3900 mmol), the mixture was stirred at 25° C. for 1 h. LCMS showed a peak with desired mass. The residue was purified by prep-HPLC (column: Unisil 3-100 C18 Ultra 150*50 mm*3 um; mobile phase: [water (0.225% FA)-ACN]; B %: 1%-30%, 10 min) followed by lyophilization to give 2-(oxazol-5-yl)-N-(tetrahydro-2H-pyran-4-yl)-1H-pyrrolo[3,2-c]pyridin-6-amine (13.31 mg, 0.0457 mmol, 11.851% yield, formic acid) as a yellow solid. MS (ESI): m/z 285.1 [M+1]⁺; ¹H NMR (400 MHz, DMSO-d₆) δ 12.03-12.00 (m, 1H), 8.54 (s, 1H), 8.43 (s, 1H), 8.13 (s, 0.6H), 7.63 (s, 1H), 7.07-6.98 (m, 1H), 6.83 (s, 1H), 6.63 (s, 1H), 3.83-3.81 (m, 2H), 3.84-3.77 (m, 1H), 3.46-3.40 (m, 2H), 1.93-1.90 (m, 2H), 1.49-1.42 (m, 2H).

### Example 20: 2-(3,3-Difluorocyclobutyl)-N-(tetrahydro-2H-pyran-4-yl)-1H-pyrrolo[3,2-c]pyridin-6-amine

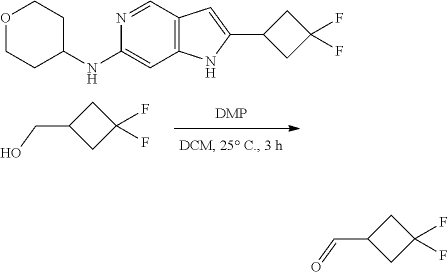

3,3-Difluorocyclobutanecarbaldehyde. To a solution of (3,3-difluorocyclobutyl)methanol (2. g, 16.38 mmol) in dichloromethane (10 mL) was added Dess-Martin periodinane (1,1,1-Tris(acetyloxy)-1,1-dihydro-1,2-benziodoxol-3-(1H)-one) (8.34 g, 19.65 mmol), the reaction mixture was stirred at 25° C. for 3 h. H NMR showed the desired product 3,3-difluorocyclobutanecarbaldehyde was detected. The mixture was poured into saturated sodium sulfite solution (40 mL). The aqueous phase was extracted with dichloromethane (20 mL×3). The combined organic phase was washed with saturated sodium bicarbonate solution (40 mL×2), dried over anhydrous sodium sulfate, filtered to give 3,3-difluorocyclobutanecarbaldehyde (1.96 g, 16.32 mmol, 99.64% yield) in 60 mL dichloromethane as a yellow liquid which was used to next step directly. ¹H NMR (400 MHz, CDCl₃) δ 9.69 (s, 1H), 2.99-2.92 (m, 1H), 2.76-2.70 (m, 4H).

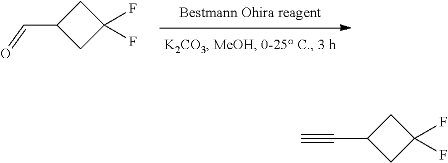

3-Ethynyl-1,1-difluorocyclobutane. To a solution of 3,3-difluorocyclobutanecarbaldehyde (1.96 g, 16.32 mmol) and 1-diazo-1-dimethoxyphosphoryl-propan-2-one (4. g, 20.82 mmol) in methanol (10 mL) at 0° C. was added potassium carbonate (9. g, 65.12 mmol). the reaction mixture was stirred at 0° C. for 2 h, then the reaction mixture was stirred at 25° C. for 1 h. H NMR showed the desired product 3-ethynyl-1,1-difluoro-cyclobutane was detected. The mixture was poured into water (40 mL). The aqueous phase was extracted with n-pentane (20 mL×3). The combined organic phase was washed with brine (30 mL×2), dried over anhydrous sodium sulfate, filtered to give 3-ethynyl-1,1-difluoro-cyclobutane (1.89 g, 16.278 mmol, 99.742% yield) in 60 mL n-pentane as a colorless liquid which was used to next step directly.

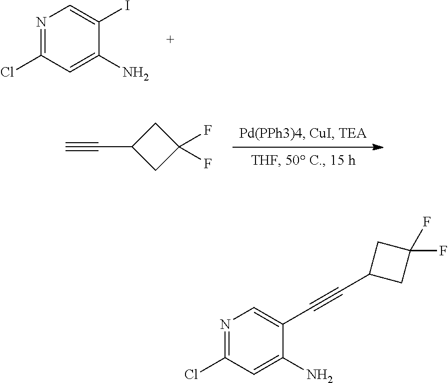

2-Chloro-5-((3,3-difluorocyclobutyl)ethynyl)pyridin-4-amine. To a solution of 2-chloro-5-iodo-pyridin-4-amine (1. g, 3.93 mmol), 3-ethynyl-1,1-difluoro-cyclobutane (2. g, 17.23 mmol) and triethylamine (3969.2 mg, 39.3 mmol) in THE (5 mL) was added cuprous iodide (75. mg, 0.3900 mmol) and tetrakis[triphenylphosphine]palladium (454.3 mg, 0.3900 mmol), the reaction mixture was stirred at 50° C. for 15 h under nitrogen. LCMS showed a peak (34.4%) with desired mass. The mixture was cooled and concentrated in reduced pressure. The residue was poured into water (40 mL). The aqueous phase was extracted with ethyl acetate (40 mL×3). The combined organic phase was washed with brine (40 mL×2), dried over anhydrous sodium sulfate, filtered and concentrated under vacuum. The residue was purified by silica gel chromatography (200-300 mesh silica gel, Petroleum ether/Ethyl acetate=100/1, 1/1, Rf=0.4, Petroleum ether/Ethyl acetate=3/1) to give 2-chloro-5-((3,3-difluorocyclobutyl)ethynyl)pyridin-4-amine (900 mg, 2.2996 mmol, 58.516% yield) as a yellow oil. MS (ESI): m/z 242.1 [M+1]⁺.

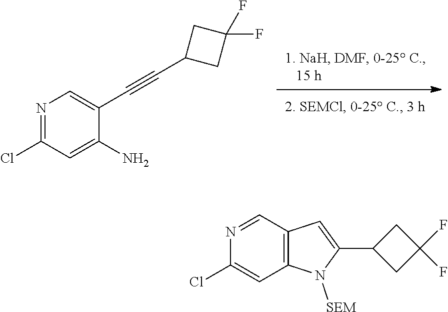 [SMILES: Nc1cc(Cl)ncc1C#CC1CC(F)(F)C1 ; C[Si](C)(C)CCOCn1c(C2CC(F)(F)C2)cc2cnc(Cl)cc21]

6-Chloro-2-(3,3-difluorocyclobutyl)-1-((2-(trimethylsilyl)ethoxy)methyl)-1H-pyrrolo[3,2-c]pyridine. To a solution of 2-chloro-5-((3,3-difluorocyclobutyl)ethynyl)pyridin-4-amine (900. mg, 3.71 mmol) in DMF (5 mL) at 0° C. was added hydrogen sodium (445.09 mg, 11.13 mmol, 60% purity), the reaction mixture was stirred at 25° C. for 15 h, after that (2-(chloromethoxy)ethyl)trimethylsilane (1236.74 mg, 7.42 mmol) was added, the reaction mixture was stirred at 25° C. for another 3 h. LCMS showed 2-chloro-5-((3,3-difluorocyclobutyl)ethynyl)pyridin-4-amine was consumed completely and a peak (66.8%) with desired mass. The mixture was poured into water (40 mL). The aqueous phase was extracted with ethyl acetate (40 mL×3). The combined organic phase was washed with brine (40 mL×2), dried over anhydrous sodium sulfate, filtered and concentrated under vacuum. The residue was purified by silica gel chromatography (200-300 mesh silica gel, Petroleum ether/Ethyl acetate=100/1, 3/1, Rf=0.6, Petroleum ether/Ethyl acetate=3/1) to give 6-chloro-2-(3,3-difluorocyclobutyl)-1-((2-(trimethylsilyl)ethoxy)methyl)-1H-pyrrolo[3,2-c]pyridine (500 mg, 0.8715 mmol, 23.497% yield) as a yellow oil. MS (ESI): m/z 373.2 [M+1]⁺.

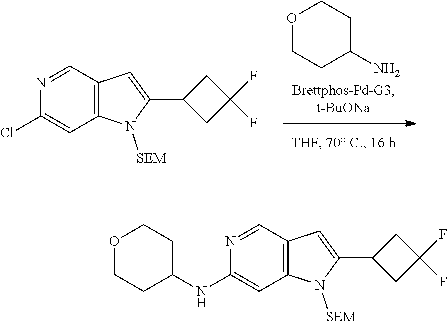

2-(3,3-Difluorocyclobutyl)-N-(tetrahydro-2H-pyran-4-yl)-1-((2-(trimethylsilyl)ethoxy)methyl)-1H-pyrrolo[3,2-c]pyridin-6-amine. To a solution of 6-chloro-2-(3,3-difluorocyclobutyl)-1-((2-(trimethylsilyl)ethoxy)methyl)-1H-pyrrolo[3,2-c]pyridine (200. mg, 0.5400 mmol) and tetrahydro-2H-pyran-4-amine (160. mg, 1.58 mmol) in THF (5 mL) was added sodium tert-butoxide (1.5 mL, 1.5 mmol, 1 M in THF) and methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (100. mg, 0.1100 mmol), the reaction mixture was stirred at 70° C. for 15 h under nitrogen. LCMS showed the material was consumed completely and a peak (68.4%) with desired mass. The mixture was cooled and concentrated in reduced pressure. The residue was poured into water (40 mL). The aqueous phase was extracted with ethyl acetate (40 mL×3). The combined organic phase was washed with brine (40 mL×2), dried over anhydrous sodium sulfate, filtered and concentrated under vacuum. Then purified by prep-TLC (Petroleum ether/Ethyl acetate=1/1, RF=0.1) to give 2-(3,3-difluorocyclobutyl)-N-tetrahydropyran-4-yl-1-(2-trimethylsilylethoxymethyl)pyrrolo[3,2-c]pyridin-6-amine (180 mg, 0.3665 mmol, 68.336% yield as a yellow solid. LCMS (ESI): m/z 438.2 [M+1]⁺.

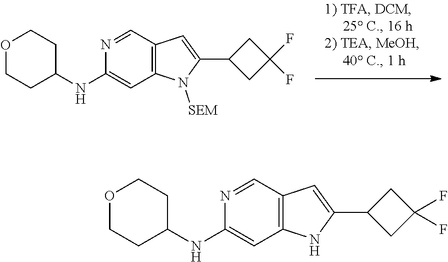 [SMILES: C[Si](C)(C)CCOCn1c(C2CC(F)(F)C2)cc2cnc(NC3CCOCC3)cc21 ; FC1(F)CC(c2cc3cnc(NC4CCOCC4)cc3[nH]2)C1]

2-(3,3-Difluorocyclobutyl)-N-(tetrahydro-2H-pyran-4-yl)-1H-pyrrolo[3,2-c]pyridin-6-amine. To a solution of 2-(3,3-difluorocyclobutyl)-N-tetrahydropyran-4-yl-1-(2-trimethylsilylethoxymethyl)pyrrolo[3,2-c]pyridin-6-amine (180. mg, 0.4100 mmol) in dichloromethane (5 mL) was added trifluoroacetic acid (5. mL, 0.4100 mmol), the reaction mixture was stirred at 25° C. for 15 h. LCMS showed 2-(3,3-difluorocyclobutyl)-N-tetrahydropyran-4-yl-1-(2-trimethylsilylethoxymethyl)pyrrolo[3,2-c]pyridin-6-amine was consumed completely and a peak (74.3%) with mass of 2-(3,3-difluorocyclobutyl)-6-((tetrahydro-2H-pyran-4-yl)amino)-1H-pyrrolo[3,2-c]pyridin-1-ol, the mixture was concentrated in reduced pressure. To the residue in methanol (5 mL) was added triethylamine (5. mL, 35.87 mmol), the reaction mixture was stirred at 40° C. for another 1 h. LCMS showed a peak (84%) with desired mass. The reaction mixture was concentrated in reduced pressure. The residue was purified by prep-HPLC (column: Phenomenex Synergi C18 150*25 mm*10 um; mobile phase: [water (0.225% FA)-ACN]; B %: 12%-42%, 10 min) followed by lyophilization to give 2-(3,3-difluorocyclobutyl)-N-(tetrahydro-2H-pyran-4-yl)-1H-pyrrolo[3,2-c]pyridin-6-amine (15.21 mg, 0.0494 mmol, 12.019% yield, formic acid) as a yellow solid. ¹H NMR (400 MHz, DMSO-d₆) δ 11.18-11.10 (m, 1H), 8.24 (s, 1H), 8.14 (s, 0.5H), 6.47 (s, 1H), 6.36-6.33 (m, H), 6.28 (s, 1H), 3.80-3.79 (m, 2H), 3.78-3.77 (m, 1H), 3.42-3.41 (m, 2H), 3.02-2.98 (m, 3H), 2.79-2.77 (m, 2H), 1.91-1.87 (m, 2H), 1.45-1.41 (m, 2H). MS (ESI): m/z 308.3 [M+1]⁺.

### Example 21: 2-(Pyridin-4-yl)-N-(tetrahydro-2H-pyran-4-yl)-1H-pyrrolo[3,2-c]pyridin-6-amine

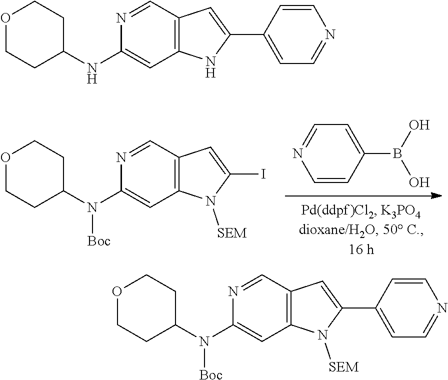

tert-Butyl(2-(pyridin-4-yl)-1-((2-(trimethylsilyl)ethoxy)methyl)-1H-pyrrolo[3,2-c]pyridin-6-yl)(tetrahydro-2H-pyran-4-yl)carbamate. To a solution of tert-butyl (2-iodo-1-((2-(trimethylsilyl)ethoxy)methyl)-1H-pyrrolo[3,2-c]pyridin-6-yl)(tetrahydro-2H-pyran-4-yl)carbamate (350 mg, 0.61 mmol), 4-pyridylboronic acid (225.03 mg, 1.83 mmol,) and potassium phosphate (388.60 mg, 1.83 mmol) in 1-4dioxane (8 mL) and water (0.4 mL) was added 1,1′-bis(diphenylphosphino)ferrocene-palladium(ii)dichloride dichloromethane complex(49.84 mg, 0.061 mmol). The mixture was stirred at 50° C. for 16 h. LCMS showed (34%) of tert-butylN-[2-iodo-1-(2-trimethylsilylethoxymethyl)pyrrolo[3,2-c]pyridin-6-yl]-N-tetrahydropyran-4-yl-carbamate was remained. Several new peaks were shown on LCMS and ˜35% of desired compound was detected. The reaction mixture was filtered and concentrated under reduced pressure to give a residue. The residue was purified by flash silica gel chromatography (ISCO®; 20 g SepaFlash® Silica Flash Column, Eluent of 0˜100% Ethyl acetate/Petroleum ether gradient @ 35 mL/min) to give tert-butyl (2-(pyridin-4-yl)-1-((2-(trimethylsilyl)ethoxy)methyl) -1H-pyrrolo[3,2-c]pyridin-6-yl)(tetrahydro-2H-pyran-4-yl)carbamate (139 mg, 0.26 mmol, 42.18% yield, 97.17% purity) as a yellow oil. ¹H NMR (400 MHz, MeOD-d₄) δ=8.81-8.80 (m, 1H), 8.70-8.68 (m, 2H), 7.84-7.83 (m, 2H), 7.54 (s, 1H), 7.01 (s, 1H), 5.66 (s, 2H), 4.38-4.35 (m, 1H), 3.94-3.90 (m, 2H), 3.59-3.56 (m, 2H), 3.51-3.45 (m, 2H), 1.95-1.91 (m, 2H), 1.60-1.54 (m, 2H), 1.39 (s, 9H), 0.90-0.86 (m, 2H), -0.07 (s, 9H).

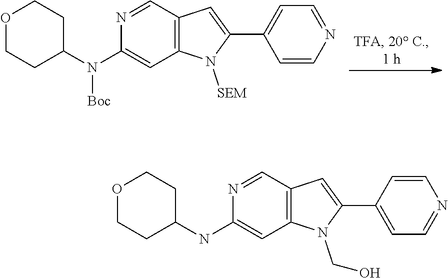 [SMILES: CC(C)(C)OC(=O)N(c1cc2c(cn1)cc(-c1ccncc1)n2COCC[Si](C)(C)C)C1CCOCC1 ; OCn1c(-c2ccncc2)cc2cnc(NC3CCOCC3)cc21]

(2-(Pyridin-4-yl)-6-((tetrahydro-2H-pyran-4-yl)amino)-1H-pyrrolo[3,2-c]pyridin-1-yl)methanol. To a solution of tert-butyl(2-(pyridin-4-yl)-1-((2-(trimethylsilyl)ethoxy)methyl) -1H-pyrrolo[3,2-c]pyridin-6-yl)(tetrahydro-2H-pyran-4-yl)carbamate (139 mg, 0.26 mmol,) in trifluoroacetic acid (2 mL) was stirred at 20° C. for 1 h. LCMS showed 7% of tert-butyl(2-(pyridin-4-yl)-1-((2-(trimethylsilyl)ethoxy)methyl)-1H-pyrrolo[3,2-c]pyridin-6-yl)(tetrahydro-2H-pyran-4-yl)carbamate was remained and one main peak with desired m/z or desired mass was detected. The reaction mixture was concentrated under reduced pressure to give a residue. The crude product (2-(pyridin-4-yl)-6-((tetrahydro-2H-pyran-4-yl)amino)-1H-pyrrolo[3,2-c]pyridin-1-yl)methanol (90 mg, 0.25 mmol, 94.27% yield, 90% purity) as a yellow oil and was used into the next step without further purification.

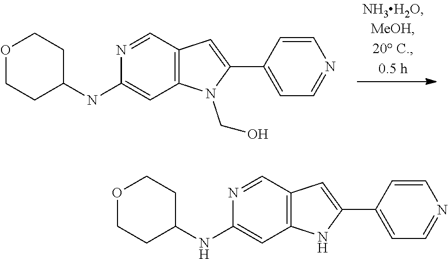

2-(Pyridin-4-yl)-N-(tetrahydro-2H-pyran-4-yl)-1H-pyrrolo[3,2-c]pyridin-6-amine. To a solution of (2-(pyridin-4-yl)-6-((tetrahydro-2H-pyran-4-yl)amino)-1H-pyrrolo[3,2-c]pyridin-1-yl)methanol (90 mg, crude) in methanol (2 mL) was added ammonium hydroxide (4 mL). The mixture was stirred at 20° C. for 0.5 h. LCMS showed (2-(pyridin-4-yl)-6-((tetrahydro-2H-pyran-4-yl)amino)-1H-pyrrolo[3,2-c]pyridine-1-yl)methanol was consumed completely and one main peak with desired m/z or desired mass was detected. The mixture concentrated under reduced pressure to remove solvent. The residue was purified by prep-HPLC (column: Boston Prime C18 150*30 mm*5 um; mobile phase: [water (0.05% ammonia hydroxide v/v)-ACN]; B %: 20%-60%, 7 min) followed by lyophilization to give 2-(pyridin-4-yl)-N-(tetrahydro-2H-pyran-4-yl)-1H-pyrrolo[3,2-c]pyridin-6-amine (42.2 mg, 0.14 mmol, 51.31% yield, 99.29% purity) as a yellow solid. ¹H NMR (400 MHz, DMSO-d₆) δ 11.53 (s, 1H), 8.60-8.58 (m, 2H), 8.40 (s, 1H), 7.75-7.74 (m, 2H), 7.14 (s, 1H), 6.44 (s, 1H), 6.28 (s, 1H), 3.90-3.86 (m, 2H), 3.86-3.80 (m, 1H), 3.45-3.32 (m, 2H), 1.93-1.89 (m, 2H), 1.48-1.43 (m, 2H); MS (ESI) m/z 295.1 [M+1]⁺.

### Example 22: (S)-2-(2-Methylpyridin-4-yl)-N-(tetrahydro-2H-pyran-3-yl)-1H-pyrrolo[3,2-c]pyridin-6-amine

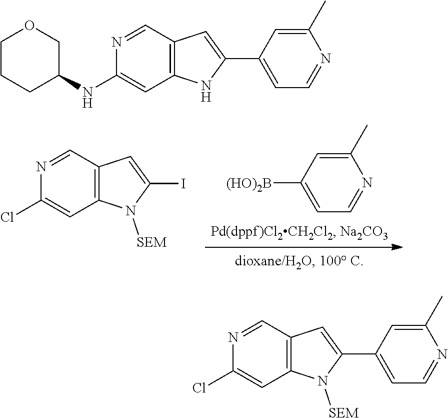

6-Chloro-2-(2-methylpyridin-4-yl)-1-((2-(trimethylsilyl)ethoxy)methyl)-1H-pyrrolo[3,2-c]pyridine. A mixture of 6-chloro-2-iodo-1-((2-(trimethylsilyl)ethoxy)methyl)-1H-pyrrolo[3,2-c]pyridine (2 g, 4.89 mmol), (2-methylpyridin-4-yl)boronic acid (670.08 mg, 4.89 mmol), 1,1′-bis(diphenylphosphino)ferrocene-palladium(ii)dichloride dichloromethane complex (399.59 mg, 0.489 mmol), sodium carbonate (1.04 g, 9.79 mmol) in 1,4-dioxane (30 mL) and water (3 mL) was degassed and purged with nitrogen for 3 times, and then the mixture was stirred at 100° C. for 16 h under nitrogen atmosphere. LCMS showed 6-chloro-2-iodo-1-((2-(trimethylsilyl)ethoxy)methyl)-1H-pyrrolo[3,2-c]pyridine was consumed completely and one main peak with desired mass was detected. The mixture was added ethyl acetate (50 mL) and filtrated, the filtrate was concentrated to afford residue. The residue was purified by flash silica gel chromatography (ISCO®; 80 g SepaFlash® Silica Flash Column, Eluent of 0˜80% Ethyl acetate/Petroleum ether gradient @ 50 mL/min) to give 6-chloro-2-(2-methylpyridin-4-yl)-1-((2-(trimethylsilyl)ethoxy)methyl)-1H-pyrrolo[3,2-c]pyridine(1.28 g, 3.31 mmol, 67.64% yield) as a brown solid. ¹H NMR (400 MHz, CDCl₃) δ 8.72 (s, 1H), 8.64-8.62 (m, 1H), 7.45-7.44 (m, 2H), 7.40-7.38 (m, 1H), 6.78 (s, 1H), 5.41 (s, 2H), 3.59-3.55 (m, 2H), 2.66 (s, 3H), 0.96-0.92 (m, 2H), −0.01 (s, 9H).

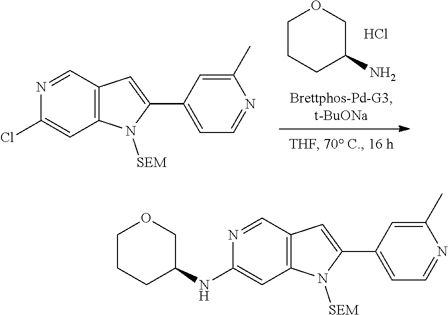

(S)-2-(2-Methylpyridin-4-yl)-N-(tetrahydro-2H-pyran-3-yl)-1-((2-(trimethylsilyl)ethoxy)methyl)-1H-pyrrolo[3,2-c]pyridin-6-amine. A mixture of 6-chloro-2-(2-methylpyridin-4-yl)-1-((2-(trimethylsilyl)ethoxy)methyl)-1H-pyrrolo[3,2-c]pyridine (400 mg, 1.07 mmol), (S)-tetrahydro-2H-pyran-3-amine hydrochloride (147.19 mg, 1.07 mmol), Methanesulfonato(2-dicyclohexylphosphino-3,6-dimethoxy-2,4,6-tri-i-propyl-1,1-biphenyl)(2-amino-1,1-biphenyl-2-yl)palladium(II) (96.96 mg, 0.106 mmol), sodium 2-methylpropan-2-olate (308.39 mg, 3.21 mmol) in THE (10 mL) was degassed and purged with nitrogen for 3 times, and then the mixture was stirred at 80° C. for 16 h under nitrogen atmosphere. LCMS showed 6-chloro-2-(2-methylpyridin-4-yl)-1-((2-(trimethylsilyl)ethoxy)methyl)-1H-pyrrolo[3,2-c]pyridine was consumed completely and one main peak with desired mass was detected. The mixture was diluted with ethyl acetate (50 mL) and filtrated, the filtrate was concentrated to afford residue. The residue was purified by flash silica gel chromatography (ISCO®; 25 g SepaFlash® Silica Flash Column, Eluent of 0˜10% methanol/Ethyl acetate @ 25 mL/min) to give (S)-2-(2-methylpyridin-4-yl)-N-(tetrahydro-2H-pyran-3-yl)-1-((2-(trimethylsilyl)ethoxy)methyl)-1H-pyrrolo[3,2-c]pyridin-6-amine(300 mg, 0.663 mmol, 62.02% yield) as a yellow gum. ¹HNMR (400 MHz, CDCl₃) δ 8.57-8.56 (m, 1H), 8.46 (d, J=1.0 Hz, 1H), 7.42 (s, 1H), 7.37-7.36 (m, 1H), 6.65 (s, 1H), 6.40 (s, 1H), 5.34 (s, 2H), 4.67-6.64 (m, 1H), 3.77-3.74 (m, 4H), 3.59-3.55 (m, 2H), 3.41-3.39 (m, 1H), 2.63 (s, 3H), 1.88-1.84 (m, 2H), 1.77-1.62 (m, 2H), 0.96-0.92 (m, 2H), -0.01 (s, 9H).

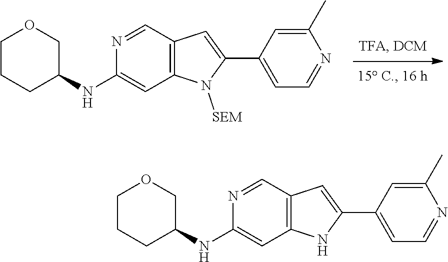 [SMILES: Cc1cc(-c2cc3cnc(N[C@H]4CCCOC4)cc3n2COCC[Si](C)(C)C)ccn1 ; Cc1cc(-c2cc3cnc(N[C@H]4CCCOC4)cc3[nH]2)ccn1]

(S)-2-(2-Methylpyridin-4-yl)-N-(tetrahydro-2H-pyran-3-yl)-1H-pyrrolo[3,2-c]pyridin-6-amine. To a solution of (S)-2-(2-methylpyridin-4-yl)-N-(tetrahydro-2H-pyran-3-yl)-1-((2-(trimethylsilyl)ethoxy)methyl)-1H-pyrrolo[3,2-c]pyridin-6-amine (300 mg, 0.683 mmol) in dichloromethane (2 mL) was added 2,2,2-trifluoroacetic acid (2 mL), the mixture was stirred at 15° C. for 16 h. LCMS showed (S)-2-(2-methylpyridin-4-yl)-N-(tetrahydro-2H-pyran-3-yl)-1-((2-(trimethylsilyl)ethoxy)methyl)-1H-pyrrolo[3,2-c]pyridin-6-amine was consumed completely and one main peak with desired mass was detected. The mixture was added ammonia hydrate (5 mL), stirred for 1 h then diluted with water (20 mL) and extracted with dichloromethane (20 mL×3), the combined organic layer was dried over sodium sulfate and concentrated to afford residue. The residue was purified by prep-HPLC (column: YMC-Actus Triart C18 150*30 mm*5 um; mobile phase: [water (0.05% ammonia hydroxide v/v)-ACN];B %: 32%-52%,10 min), then dried by lyophilization to give (S)-2-(2-methylpyridin-4-yl)-N-(tetrahydro-2H-pyran-3-yl)-1H-pyrrolo[3,2-c]pyridin-6-amine (88.8 mg, 0.285 mmol, 41.62% yield, 98.85% purity) as a yellow solid. ¹H NMR (400 MHz, DMSO-d₆) δ 11.31 (s, 1H), 8.43 (d, J=5.3 Hz, 1H), 8.35 (s, 1H), 7.61 (s, 1H), 7.53 (dd, J=1.4, 5.2 Hz, 1H), 7.05 (d, J=1.0 Hz, 1H), 6.39 (s, 1H), 5.95 (d, J=7.9 Hz, 1H), 3.93-3.92 (m, 1H), 3.86-3.78 (m, 1H), 3.75-3.72 (m, 1H), 3.363.32 (m, 1H), 3.11-3.09 (m, 1H), 2.51 (s, 3H), 2.06-1.90 (m, 1H), 1.79-1.66 (m, 1H), 1.65-1.41 (m, 2H). MS (ESI) m/z 309.1 [M+1]⁺.

### Example 23: N-(Tetrahydro-2H-pyran-4-yl)-2-(o-tolyl)-1H-pyrrolo[3,2-c]pyridin-6-amine

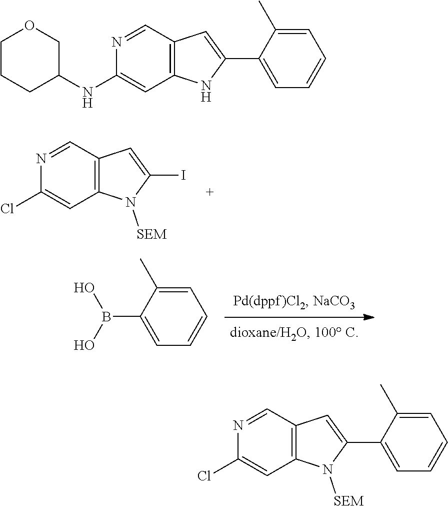

6-Chloro-2-(o-tolyl)-1-((2-(trimethylsilyl)ethoxy)methyl)-1H-pyrrolo[3,2-c]pyridine. To a mixture of 6-chloro-2-iodo-1-((2-(trimethylsilyl)ethoxy)methyl)-1H-pyrrolo[3,2-c]pyridine (0.8 g, 1.96 mmol) and o-tolylboronic acid (319.33 mg, 2.35 mmol) in 1,4-Dioxane (10 mL) and Water (1 mL) was added [1,1′-bis(diphenylphosphino) ferrocene]dichloropalladium(ii) (143.21 mg, 0.20 mmol) and sodium carbonate (414.89 mg, 3.91 mmol). The mixture was stirred at 100° C. for 16 h under nitrogen atmosphere. LCMS showed 2-[(6-chloro-2-iodo-pyrrolo[3,2-c]pyridin-1-yl)methoxy]ethyl-trimethyl-silane was consumed completely and a peak (68%) with desired mass. The mixture was concentrated under reduced pressure to give a residue. The residue was purified by silica gel chromatography (100-200 mesh silica gel, Petroleum ether/Ethyl acetate=0/1, 20/1) to give 6-chloro-2-(o-tolyl)-1-((2-(trimethylsilyl)ethoxy)methyl)-1H-pyrrolo[3,2-c]pyridine (570 mg, 1.53 mmol, 78.09% yield) as a yellow oil. MS (ESI): m/z 373.0 [M+1]⁺.

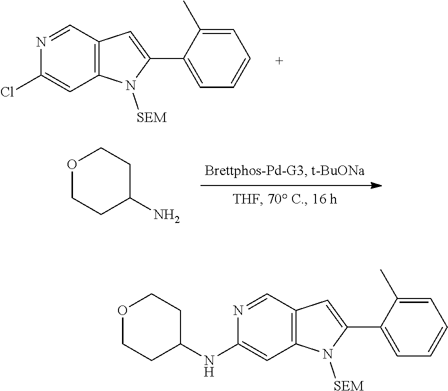 [SMILES: Cc1ccccc1-c1cc2cnc(Cl)cc2n1COCC[Si](C)(C)C ; NC1CCOCC1 ; Cc1ccccc1-c1cc2cnc(NC3CCOCC3)cc2n1COCC[Si](C)(C)C]

N-(Tetrahydro-2H-pyran-4-yl)-2-(o-tolyl)-1-((2-(trimethylsilyl)ethoxy)methyl)-1H-pyrrolo[3,2-c]pyridin-6-amine. To a mixture of 6-chloro-2-(o-tolyl)-1-((2-(trimethylsilyl)ethoxy)methyl) -1H-pyrrolo[3,2-c]pyridine (570. mg, 1.53 mmol) and tetrahydro-2H-pyran-4-amine (309.18 mg, 3.06 mmol) in THE (10 mL)was added Sodium tert-Butoxide (293.74 mg, 3.06 mmol) and Methanesulfonato(2-dicyclohexylphosphino-3,6-dimethoxy-2,4,6-tri-i-propyl-1,1-biphenyl)(2-amino-1,1-biphenyl-2-yl)palladium(II) (138.54 mg, 0.15 mmol). The mixture was stirred at 80° C. for 16 h under nitrogen atmosphere. LCMS showed 6-chloro-2-(o-tolyl)-1-((2-(trimethylsilyl)ethoxy)methyl)-1H-pyrrolo[3,2-c]pyridine was consumed completely and a peak (71%) with desired mass. The mixture was concentrated under reduced pressure to give a residue. The residue was purified by silica gel chromatography (100-200 mesh silica gel, Petroleum ether/Ethyl acetate=20/1, 1/1) to give N-(tetrahydro-2H-pyran-4-yl)-2-(o-tolyl)-1-((2-(trimethylsilyl)ethoxy)methyl)-1H-pyrrolo[3,2-c]pyridin-6-amine (600 mg, 1.371 mmol, 89.704% yield) as a yellow oil. MS (ESI): m/z 438.3 [M+1]⁺.

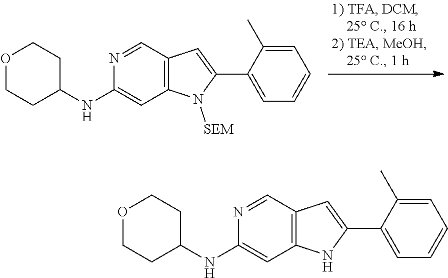 [SMILES: Cc1ccccc1-c1cc2cnc(NC3CCOCC3)cc2n1COCC[Si](C)(C)C ; Cc1ccccc1-c1cc2cnc(NC3CCOCC3)cc2[nH]1]

N-(Tetrahydro-2H-pyran-4-yl)-2-(o-tolyl)-1H-pyrrolo[3,2-c]pyridin-6-amine. To a mixture of N-(tetrahydro-2H-pyran-4-yl)-2-(o-tolyl)-1-((2-(trimethylsilyl) ethoxy)methyl)-1H-pyrrolo[3,2-c]pyridin-6-amine (600 mg, 1.37 mmol) in chloromethane (5 mL) was added Trifluoroacetic acid (5. mL, 1.37 mmol). The mixture was stirred at 25° C. for 16 h. LCMS showed N-(tetrahydro-2H-pyran-4-yl)-2-(o-tolyl)-1-((2-(trimethylsilyl)ethoxy)methyl)-1H-pyrrolo[3,2-c]pyridin-6-amine was consumed completely and the mass of intermediate. The mixture was concentrated under reduced pressure to give a residue. To the residue in Methanol (5 mL) was added triethylamine (5. mL, 1.37 mmol), the mixture was stirred at 25° C. for 1 h. LCMS showed N-(tetrahydro-2H-pyran-4-yl)-2-(o-tolyl)-1-((2-(trimethylsilyl)ethoxy)methyl)-1H-pyrrolo[3,2-c]pyridin-6-amine was consumed completely and a peak (67%) with desired mass. The mixture was concentrated under reduced pressure to give a residue. The residue was purified by prep-HPLC (Boston Prime C18 150*25 mm*5 um; mobile phase: [water (0.05% ammonia hydroxide v/v)-ACN]; B %: 43%-65%, 9 min) followed by lyophilization to give N-(tetrahydro-2H-pyran-4-yl)-2-(o-tolyl) -1H-pyrrolo[3,2-c]pyridin-6-amine (90.16 mg, 0.29 mmol, 21.22% yield) as a yellow solid. Additional 2.45 mg for delivery. ¹H NMR (400 MHz, DMSO-d₆) δ=10.92 (s, 1H), 8.31 (s, 1H), 7.50 (dd, J=1.5, 7.5 Hz, 1H), 7.30-7.25 (m, 3H), 6.44 (d, J=1.2 Hz, 1H), 6.37 (s, 1H), 5.78 (d, J=8.1 Hz, 1H), 3.89-3.86 (m, 3H), 3.44-3.39 (m, 2H), 2.45 (s, 3H), 1.91-1.88 (m, 2H), 1.47-1.40 (m, 2H). MS (ESI): m/z 308.3 [M+1]⁺.

### Example 24: 1-Methyl-N-(tetrahydro-2H-pyran-4-yl)-2-(2-((2,2,2-trifluoroethyl)amino)pyrimidin-4-yl)-1H-pyrrolo[3,2-c]pyridin-6-amine

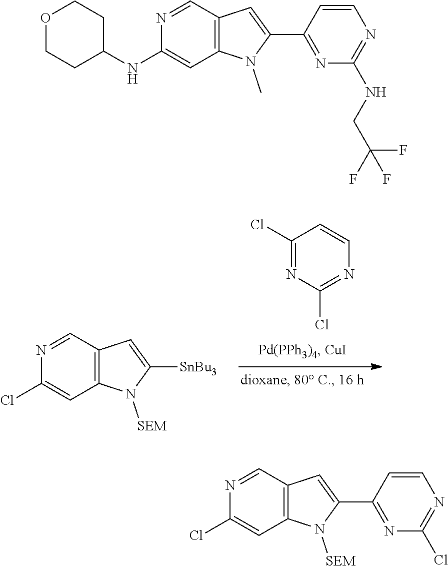

6-Chloro-2-(2-chloropyrimidin-4-yl)-1-((2-(trimethylsilyl)ethoxy)methyl)-1H-pyrrolo[3,2-c]pyridine. To a solution of 6-chloro-2-(tributylstannyl)-1-((2-(trimethylsilyl)ethoxy)methyl)-1H-pyrrolo[3,2-c]pyridine (4 g, 6.99 mmol) and 2,4-dichloropyrimidine (1042 mg, 6.99 mmol) in 1,4-Dioxane (40 mL) were added tetrakis[triphenylphosphine]palladium(0) (808 mg, 0.70 mmol) and copper iodide (133 mg, 0.70 mmol) under nitrogen, the mixture was stirred at 80° C. for 16 h. LCMS 6-chloro-2-(tributylstannyl)-1-((2-(trimethylsilyl)ethoxy)methyl)-1H-pyrrolo[3,2-c]pyridine was consumed completely and a peak (58%) with desired mass. The mixture was combined with pervious page (1 g, crude) and concentrated under vacuum. The residue was purified by flash silica gel chromatography (Biotage, 40 g SepaFlash® Silica Flash Column, Eluent of 30˜60% Ethyl acetate/Petroleum ether gradient @ 40 mL/min) to give 6-chloro-2-(2-chloropyrimidin-4-yl)-1-((2-(trimethylsilyl)ethoxy)methyl)-1H-pyrrolo[3,2-c]pyridine (2 g, 4.45 mmol, 63.6% yield) as a brown oil. ¹H NMR (400 MHz, DMSO-d₆) δ 8.87 (d, J=5.3 Hz, 1H), 8.83 (d, J=0.8 Hz, 1H), 8.16 (d, J=5.4 Hz, 1H), 7.93 (s, 1H), 7.73 (d, J=0.6 Hz, 1H), 6.12 (s, 2H), 3.44 (t, J=7.8 Hz, 2H), 0.74 (t, J=7.8 Hz, 2H), -0.20 (s, 9H); MS (ESI): m/z 394.9 [M+1]⁺.

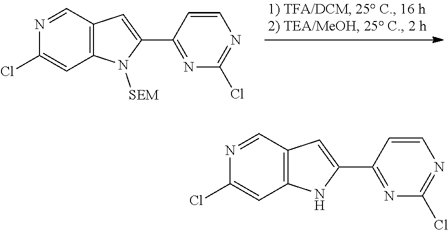 [SMILES: C[Si](C)(C)CCOCn1c(-c2ccnc(Cl)n2)cc2cnc(Cl)cc21 ; Clc1cc2[nH]c(-c3ccnc(Cl)n3)cc2cn1]

6-Chloro-2-(2-chloropyrimidin-4-yl)-1H-pyrrolo[3,2-c]pyridine. To a solution of 6-chloro-2-(2-chloropyrimidin-4-yl)-1-((2-(trimethylsilyl)ethoxy)methyl)-1H-pyrrolo[3,2-c]pyridine (400. mg, 1.01 mmol) in dichloromethane (10 mL) was added trifluoroacetic acid (10. mL, 1.01 mmol), the mixture was stirred at 25° C. for 16 h. LCMS showed 6-chloro-2-(2-chloropyrimidin-4-yl)-1-((2-(trimethylsilyl)ethoxy)methyl)-1H-pyrrolo[3,2-c]pyridine was consumed completely and a peak (76%) with the mass of intermediate. The mixture was concentrated under oil pump. To the residue in methanol (5 mL) was added triethylamine (5. mL), the mixture was stirred at 25° C. for 2 h. LCMS showed a peak (72%) with desired mass. The mixture was concentrated under oil pump. The residue was triturated with ethyl acetate (10 mL) and stirred for 1 h. The suspension was filtered and the filter cake was washed with methanol (5 mL) and the filter cake was concentrated under vacuum. The filtrated was concentrated under vacuum, and purified by flash silica gel chromatography (Biotage, 12 g SepaFlash® Silica Flash Column, Eluent of 30˜100% Ethyl acetate/Petroleum ether gradient @30 mL/min), then combined with the filter cake to give 6-chloro-2-(2-chloropyrimidin-4-yl)-1H-pyrrolo[3,2-c]pyridine (200 mg, 0.6262 mmol, 61.892% yield) as a yellow solid. ¹H NMR (400 MHz, DMSO-d₆) δ 12.46 (s, 1H), 8.83-8.79 (m, 2H), 8.17 (d, J=5.3 Hz, 1H), 7.70 (d, J=1.1 Hz, 1H), 7.47 (s, 1H); MS (ESI): m/z 264.8 [M+1]⁺.

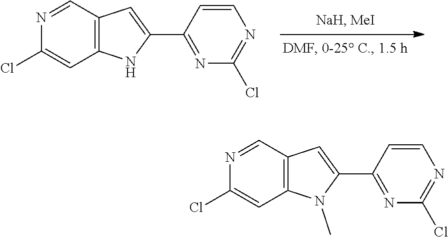 [SMILES: Clc1cc2[nH]c(-c3ccnc(Cl)n3)cc2cn1 ; Cn1c(-c2ccnc(Cl)n2)cc2cnc(Cl)cc21]

6-Chloro-2-(2-chloropyrimidin-4-yl)-1-methyl-1H-pyrrolo[3,2-c]pyridine. To a solution of 6-chloro-2-(2-chloropyrimidin-4-yl)-1H-pyrrolo[3,2-c]pyridine (200. mg, 0.7500 mmol) in DMF (10 mL) was added sodium hydride (60.35 mg, 1.51 mmol) at 0° C., the mixture was stirred at 25° C. for 0.5 h. Then iodomethane (160.63 mg, 1.13 mmol) was added to the mixture at 0° C., and stirred at 25° C. for 1 h. LCMS showed 6-chloro-2-(2-chloropyrimidin-4-yl)-1H-pyrrolo[3,2-c]pyridine remained (6%) and a peak (49%) with desired mass. The mixture was poured into saturated ammonium chloride aqueous (30 mL). The aqueous phase was extracted with ethyl acetate (20 mL×2). The combined organic phase was washed with brine (10 mL×2), dried over anhydrous sodium sulfate, filtered and concentrated under vacuum. The residue was purified by prep-TLC (Petroleum ether:Ethyl acetate=0:1) to give 6-chloro-2-(2-chloropyrimidin-4-yl)-1-methyl-1H-pyrrolo[3,2-c]pyridine (100 mg, 0.2719 mmol, 36.044% yield) as a yellow solid. MS (ESI): m/z 279.1 [M+1]⁺.

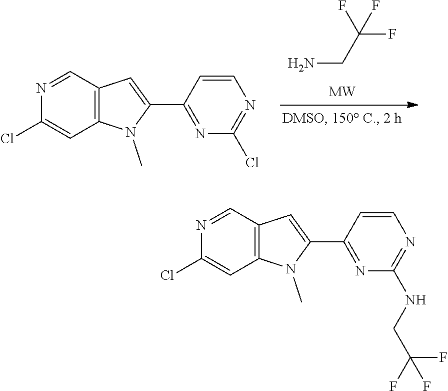

4-(6-Chloro-1-methyl-1H-pyrrolo[3,2-c]pyridin-2-yl)-N-(2,2,2-trifluoroethyl)pyrimidin-2-amine. To a solution of 6-chloro-2-(2-chloropyrimidin-4-yl)-1-methyl-1H-pyrrolo[3,2-c]pyridine (100. mg, 0.3600 mmol) in anhydride DMSO (3 mL) was added 2,2,2-trifluoroethanamine (0.61 mL, 10.75 mmol) under nitrogen, the mixture was stirred at 150° C. for 2 h under microwave. LCMS showed 6-chloro-2-(2-chloropyrimidin-4-yl)-1-methyl-1H-pyrrolo[3,2-c]pyridine was consumed completely and a peak (72%) with desired mass. The mixture was cooled and poured into water (50 mL). The aqueous phase was extracted with ethyl acetate (30 mL×2). The combined organic phase was washed with brine (10 mL×2), dried over anhydrous sodium sulfate, filtered and concentrated under vacuum. The residue was purified by flash silica gel chromatography (Biotage, 4 g SepaFlash® Silica Flash Column, Eluent of 20˜60% Ethyl acetate/Petroleum ether gradient @ 30 mL/min) to give 4-(6-chloro-1-methyl-1H-pyrrolo[3,2-c]pyridin-2-yl)-N-(2,2,2-trifluoroethyl)pyrimidin-2-amine (100 mg, 0.1943 mmol, 54.236% yield) as a yellow solid. MS (ESI): m/z 341.9 [M+1]⁺.

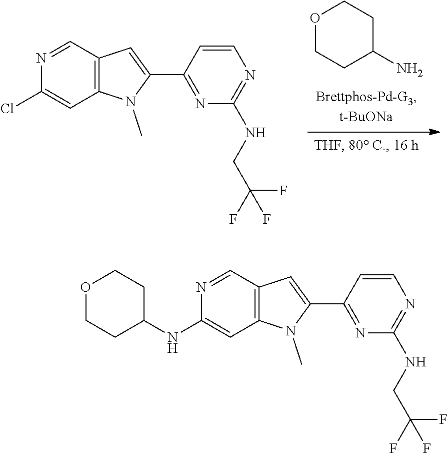

1-Methyl-N-(tetrahydro-2H-pyran-4-yl)-2-(2-((2,2,2-trifluoroethyl)amino)pyrimidin-4-yl)-1H-pyrrolo[3,2-c]pyridin-6-amine. To a solution of 4-(6-chloro-1-methyl-1H-pyrrolo[3,2-c]pyridin-2-yl)-N-(2,2,2-trifluoroethyl)pyrimidin-2-amine (100 mg, 0.29 mmol) and tetrahydropyran-4-amine (88.8 mg, 0.88 mmol) in THE (3 mL) were added Sodium tert-Butoxide (0.29 mL, 0.59 mmol, 2 M in THF) and Methanesulfonato(2-dicyclohexylphosphino-3,6-dimethoxy-2,4,6-tri-i-propyl-1,1-biphenyl)(2-amino-1,1-biphenyl-2-yl)palladium(II) (26.53 mg, 0.0300 mmol) under nitrogen, the mixture was stirred at 80° C. for 16 h. LCMS showed 4-(6-chloro-1-methyl-1H-pyrrolo[3,2-c]pyridin-2-yl)-N-(2,2,2-trifluoroethyl)pyrimidin-2-amine was consumed completely and a peak (58%) with desired mass. The mixture was diluted with ethyl acetate (100 mL) and filtered with a pad of silica gel (100-200 mesh), then washed with dichloromethane:methanol=10:1 (100 ml), the filtrate was concentrated under vacuum. The residue was purified by prep-HPLC (column: Phenomenex luna C18 150*25 mm*10 um; mobile phase: [water (0.225% FA) -ACN]; B %: 8%-38%, 10 min), then dried by lyophilization to give 1-methyl-N-(tetrahydro-2H-pyran-4-yl)-2-(2-((2,2,2-trifluoroethyl)amino)pyrimidin-4-yl)-1H-pyrrolo[3,2-c]pyridin-6-amine (41.02 mg, 0.0987 mmol, 33.7% yield, formic acid) as a yellow solid. ¹H NMR (400 MHz, DMSO-d₆) δ 8.40 (s, 1H), 8.32 (d, J=5.3 Hz, 1H), 8.19 (s, 0.5H), 7.81 (t, J=6.6 Hz, 1H), 7.17-7.16 (m, 2H), 6.38 (s, 1H), 6.09 (d, J=8.3 Hz, 1H), 4.20-4.18 (m, 2H), 3.97 (s, 3H), 3.90-3.87 (m, 3H), 3.46-3.40 (m, 2H), 1.92-1.89 (m, 2H), 1.50-1.43 (m, 2H); MS (ESI): m/z 407.1 [M+1]⁺.

### Example 25: N-(Tetrahydro-2H-pyran-4-yl)-2-(6-((2,2,2-trifluoroethyl)amino)pyrimidin-4-yl)-1H-pyrrolo[3,2-c]pyridin-6-amine

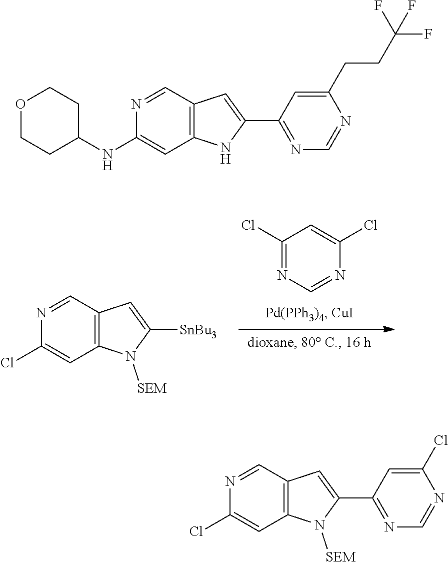

6-Chloro-2-(6-chloropyrimidin-4-yl)-1-((2-(trimethylsilyl)ethoxy)methyl)-1H-pyrrolo[3,2-c]pyridine. To a solution of 6-chloro-2-(tributylstannyl)-1-((2-(trimethylsilyl)ethoxy)methyl)-1H-pyrrolo[3,2-c]pyridine (3. g, 5.25 mmol) and 4,6-dichloropyrimidine (781.51 mg, 5.25 mmol) in 1,4-Dioxane (20 mL) were added tetrakis[triphenylphosphine]palladium(0) (606.18 mg, 0.5200 mmol) and copper iodide (99.91 mg, 0.5200 mmol) under nitrogen, the mixture was stirred at 80° C. for 16 h under nitrogen. LCMS showed 6-chloro-2-(tributylstannyl)-1-((2-(trimethylsilyl)ethoxy)methyl)-1H-pyrrolo[3,2-c]pyridine was consumed completely and a peak (38%) with desired mass. The mixture was concentrated under vacuum. The residue was purified by flash silica gel chromatography (Biotage, 25 g SepaFlash® Silica Flash Column, Eluent of 20˜50% Ethyl acetate/Petroleum ether gradient @ 60 mL/min) to give 6-chloro-2-(6-chloropyrimidin-4-yl)-1-((2-(trimethylsilyl)ethoxy)methyl)-1H-pyrrolo[3,2-c]pyridine (1.1 g, 2.7823 mmol, 53.038% yield) as a brown solid. ¹H NMR (400 MHz, DMSO-d₆) δ 9.14 (d, J=1.0 Hz, 1H), 8.81 (s, 1H), 8.33 (d, J=1.1 Hz, 1H), 7.93 (s, 1H), 7.71 (s, 1H), 6.15 (s, 2H), 3.38-3.36 (m, 2H), 0.71-0.67 (m, 2H), -0.23 (s, 9H); MS (ESI): m/z 394.9 [M+1]⁺.

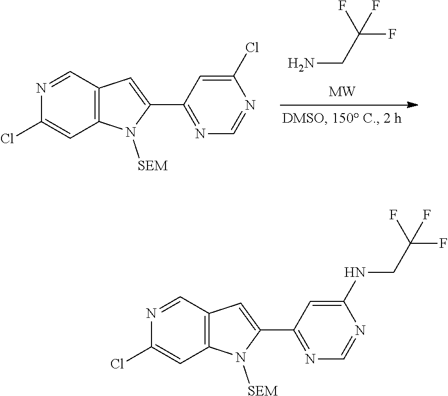

6-(6-Chloro-1-((2-(trimethylsilyl)ethoxy)methyl)-1H-pyrrolo[3,2-c]pyridin-2-yl)-N-(2,2,2-trifluoroethyl)pyrimidin-4-amine. To a solution of 6-chloro-2-(6-chloropyrimidin-4-yl)-1-((2-(trimethylsilyl)ethoxy)methyl)-1H-pyrrolo[3,2-c]pyridine (400. mg, 1.01 mmol) in DMSO (3 mL) was added 2,2,2-trifluoroethanamine (1.16 mL, 20.23 mmol) under nitrogen, the mixture was stirred at 150° C. for 2 h under microwave. LCMS showed 6-chloro-2-(6-chloropyrimidin-4-yl)-1-((2-(trimethylsilyl)ethoxy)methyl)-1H-pyrrolo[3,2-c]pyridine was consumed completely and a peak (37%) with desired mass. The mixture was cooled and poured into water (50 mL). The aqueous phase was extracted with ethyl acetate (30 mL×2). The combined organic phase was washed with brine (30 mL×2), dried over anhydrous sodium sulfate, filtered and concentrated under vacuum. The residue was purified by flash silica gel chromatography (Biotage, 4 g SepaFlash® Silica Flash Column, Eluent of 10˜60% Ethyl acetate/Petroleum ether gradient @ 20 mL/min) to give 6-(6-chloro-1-((2-(trimethylsilyl)ethoxy)methyl)-1H-pyrrolo[3,2-c]pyridin-2-yl)-N-(2,2,2-trifluoroethyl)pyrimidin-4-amine (280 mg, 0.6114 mmol, 60.433% yield) as a white solid. ¹H NMR (400 MHz, DMSO-d₆) δ 8.75 (s, 1H), 8.63 (s, 1H), 8.20 (s, 1H), 7.85 (s, 1H), 7.22 (s, 1H), 7.09 (s, 1H), 6.10 (s, 2H), 4.31-4.25 (m, 2H), 3.32-3.28 (m, 2H), 0.68-0.64 (m, 2H), -0.20-0.25 (m, 9H); MS (ESI): m/z 458.1 [M+1]⁺.

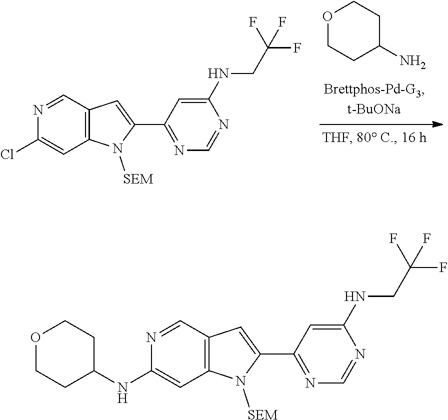

N-(Tetrahydro-2H-pyran-4-yl)-2-(6-((2,2,2-trifluoroethyl)amino)pyrimidin-4-yl)-1-((2-(trimethylsilyl)ethoxy)methyl)-1H-pyrrolo[3,2-c]pyridin-6-amine. To a solution of 6-(6-chloro-1-((2-(trimethylsilyl)ethoxy)methyl)-1H-pyrrolo[3,2-c]pyridin-2-yl)-N-(2,2,2-trifluoroethyl)pyrimidin-4-amine (280. mg, 0.6100 mmol) and tetrahydropyran-4-amine (185.54 mg, 1.83 mmol) in THF (5 mL) were added Methanesulfonato(2-dicyclohexylphosphino-3,6-dimethoxy-2,4,6-tri-i-propyl-1,1-biphenyl)(2-amino-1,1-biphenyl-2-yl)palladium(II) (55.43 mg, 0.0600 mmol) and Sodium tert-Butoxide (0.61 mL, 1.22 mmol, 2 M in THF) under nitrogen, the mixture was stirred at 80° C. for 16 h. LCMS showed 6-[6-chloro-1-(2-trimethylsilylethoxymethyl)pyrrolo[3,2-c]pyridin-2-yl]-N-(2,2,2-trifluoroethyl)pyrimidin-4-amine was consumed completely and a peak (57%) with desired mass. The mixture was concentrated under vacuum. The residue was purified by flash silica gel chromatography (Biotage, 4 g SepaFlash® Silica Flash Column, Eluent of 30˜50% Ethyl acetate/Petroleum ether gradient @ 20 mL/min) to give N-(tetrahydro-2H-pyran-4-yl)-2-(6-((2,2,2-trifluoroethyl)amino)pyrimidin-4-yl)-1-((2-(trimethylsilyl)ethoxy)methyl)-1H-pyrrolo[3,2-c]pyridin-6-amine (220 mg, 0.4117 mmol, 67.332% yield) as a yellow solid. ¹H NMR (400 MHz, DMSO-d₆) δ 8.56 (s, 1H), 8.40 (s, 1H), 8.03 (s, 1H), 7.00-6.97 (m, 2H), 6.52 (s, 1H), 6.14 (d, J=8.0 Hz, 1H), 5.94 (s, 2H), 4.31-4.22 (m, 2H), 3.89-3.86 (m, 3H), 3.45-3.42 (m, 2H), 3.33-3.28 (m, 2H), 1.91-1.88 (m, 2H), 1.50-1.41 (m, 2H), 0.71-0.67 (m, 2H), -0.20-0.22 (m, 9H); MS (ESI): m/z 523.2 [M+1]⁺.

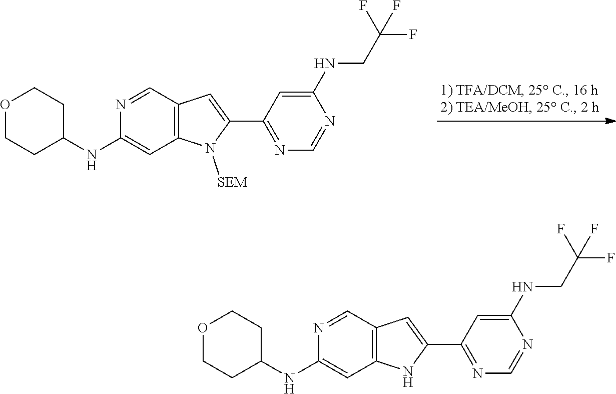 [SMILES: C[Si](C)(C)CCOCn1c(-c2cc(NCC(F)(F)F)ncn2)cc2cnc(NC3CCOCC3)cc21 ; FC(F)(F)CNc1cc(-c2cc3cnc(NC4CCOCC4)cc3[nH]2)ncn1]

N-(Tetrahydro-2H-pyran-4-yl)-2-(6-((2,2,2-trifluoroethyl)amino)pyrimidin-4-yl)-1H-pyrrolo[3,2-c]pyridin-6-amine. To a solution of N-(tetrahydro-2H-pyran-4-yl)-2-(6-((2,2,2-trifluoroethyl)amino)pyrimidin-4-yl)-1-((2-(trimethylsilyl)ethoxy)methyl)-1H-pyrrolo[3,2-c]pyridin-6-amine (220 mg, 0.42 mmol) in dichloromethane (5 mL) was added trifluoroacetic acid (5 mL, 0.42 mmol), the mixture was stirred at 25° C. for 16 h. LCMS showed N-(tetrahydro-2H-pyran-4-yl)-2-(6-((2,2,2-trifluoroethyl)amino)pyrimidin-4-yl)-1-((2-(trimethylsilyl)ethoxy)methyl)-1H-pyrrolo[3,2-c]pyridin-6-amine was consumed completely and a peak (86%) with desired mass. The mixture was concentrated under oil pump. To the residue in methanol (5 mL) was added triethylamine (5 mL, 0.42 mmol), the mixture was stirred at 25° C. for 2 h. LCMS showed a peak (90%) with desired mass. The mixture was concentrated under vacuum. The residue was purified by prep-HPLC (column: Shim-pack C18 150*25*10 um; mobile phase: [water (0.225% FA) -ACN]; B %: 12%-32%, 10 min), then dried by lyophilization. H NMR indicated impurities, then the crude product was purified by prep-HPLC (column: Unisil 3-100 C18 Ultra 150*50 mm*3 um; mobile phase: [water (0.225% FA)-ACN]; B %: 13%-33%, 10 min), then dried by lyophilization to give N-tetrahydropyran-4-yl-2-[6-(2,2,2-trifluoroethylamino)pyrimidin-4-yl]-1H-pyrrolo[3,2-c]pyridin-6-amine (71.05 mg, 0.1794 mmol, 42.63% yield) as a yellow solid. ¹H NMR (400 MHz, MeOD) δ 8.57 (d, J=1.1 Hz, 1H), 8.40 (d, J=0.8 Hz, 1H), 7.22 (s, 1H), 7.05 (s, 1H), 6.71 (s, 1H), 4.25-4.22 (qm, 2H), 4.03-4.00 (m, 2H), 3.77-3.62 (m, 1H), 3.61-3.55 (m, 2H), 2.06-2.02 (m, 2H), 1.64-1.60 (m, 2H); MS (ESI): m/z 393.3 [M+1]⁺.

### Example 26: 2-Cyclohexyl-N-(tetrahydro-2H-pyran-4-yl)-1H-pyrrolo[3,2-c]pyridin-6-amine

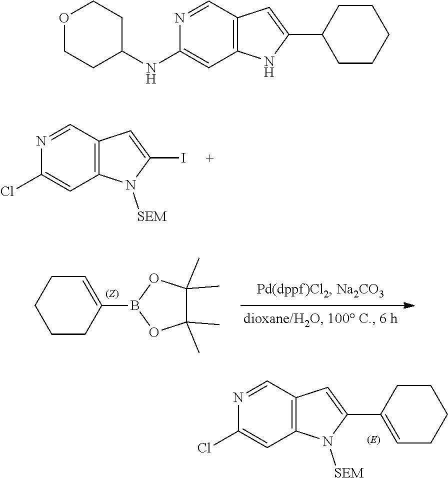 [SMILES: c1nc(NC2CCOCC2)cc2[nH]c(C3CCCCC3)cc12 ; C[Si](C)(C)CCOCn1c(I)cc2cnc(Cl)cc21 ; CC1(C)OB(C2=CCCCC2)OC1(C)C ; C[Si](C)(C)CCOCn1c(C2=CCCCC2)cc2cnc(Cl)cc21]

6-Chloro-2-(cyclohex-1-en-1-yl)-1-((2-(trimethylsilyl)ethoxy)methyl)-1H-pyrrolo[3,2-c]pyridine. To a mixture of 6-chloro-2-iodo-1-((2-(trimethylsilyl)ethoxy)methyl)-1H-pyrrolo[3,2-c]pyridine (500 mg, 1.22 mmol) and 2-(cyclohex-1-en-1-yl)-4,4,5,5-tetramethyl-1,3,2-dioxaborolane (284.9 mg, 1.47 mmol) in 1,4-Dioxane (5 mL) and Water (0.50 mL) was added [1,1′-bis(diphenylphosphino)ferrocene]dichloropalladium(ii) (89.51 mg, 0.12 mmol) and sodium carbonate (259.31 mg, 2.45 mmol). The mixture was stirred at 100° C. for 16 h under nitrogen atmosphere. LCMS showed 6-chloro-2-iodo-1-((2-(trimethylsilyl)ethoxy)methyl)-1H-pyrrolo[3,2-c]pyridine was consumed completely and a peak (46%) with desired mass. The mixture was concentrated under reduced pressure to give a residue. The residue was purified by flash silica gel chromatography (Biotage, 12 g SepaFlash® Silica Flash Column, Eluent of 0˜17% Ethyl acetate/Petroleum ether gradient @ 35 mL/min) to give 6-chloro-2-(cyclohex-1-en-1-yl)-1-((2-(trimethylsilyl)ethoxy)methyl)-1H-pyrrolo[3,2-c]pyridine (200 mg, 0.55 mmol, 45.04% yield) as a yellow oil. MS (ESI): m/z 363.1 [M+1]⁺.

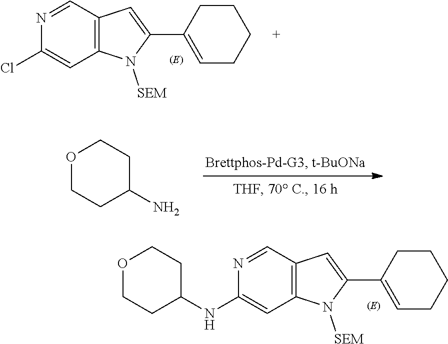

2-(Cyclohex-1-en-1-yl)-N-(tetrahydro-2H-pyran-4-yl)-1-((2-(trimethylsilyl)ethoxy)methyl)-1H-pyrrolo[3,2-c]pyridin-6-amine. To a mixture of 6-chloro-2-(cyclohex-1-en-1-yl)-1-((2-(trimethylsilyl)ethoxy)methyl) -1H-pyrrolo[3,2-c]pyridine (190 mg, 0.52 mmol) and tetrahydro-2H-pyran-4-amine (158.84 mg, 1.57 mmol) in THF (2 mL) was added Methanesulfonato (2-dicyclohexylphosphino-3,6-dimethoxy-2,4,6-tri-i-propyl-1,1-biphenyl)(2-amino-1,1-biphenyl-2-yl)palladium(II) (47.45 mg, 0.05 mmol) and Sodium tert-Butoxide (1.05 mL, 1.05 mmol). The mixture was stirred at 70° C. for 16 h under nitrogen atmosphere. LCMS showed 6-chloro-2-(cyclohex-1-en-1-yl)-1-((2-(trimethylsilyl)ethoxy)methyl)-1H-pyrrolo[3,2-c]pyridine was consumed completely and a peak (84%) with desired mass. The mixture was concentrated under reduced pressure to give a residue. The residue was purified by flash silica gel chromatography (Biotage, 12 g SepaFlash® Silica Flash Column, Eluent of 0˜₄₀% Ethyl acetate/Petroleum ether gradient @ 30 mL/min) to give 2-(cyclohex-1-en-1-yl)-N-(tetrahydro-2H-pyran-4-yl)-1-((2-(trimethylsilyl)ethoxy)methyl)-1H-pyrrolo[3,2-c]pyridin-6-amine (200 mg, 0.47 mmol, 89.34% yield) as a yellow oil. LCMS (ESI): m/z 428.3 [M+1]⁺.

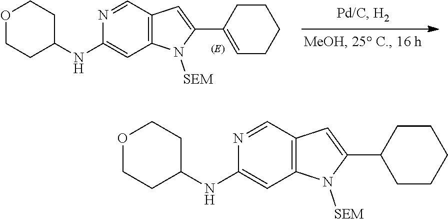 [SMILES: C[Si](C)(C)CCOCn1c(C2=CCCCC2)cc2cnc(NC3CCOCC3)cc21 ; C[Si](C)(C)CCOCn1c(C2CCCCC2)cc2cnc(NC3CCOCC3)cc21]

2-Cyclohexyl-N-(tetrahydro-2H-pyran-4-yl)-1-((2-(trimethylsilyl)ethoxy)methyl)-1H-pyrrolo[3,2-c]pyridin-6-amine. To a solution of 2-(cyclohex-1-en-1-yl)-N-(tetrahydro-2H-pyran-4-yl)-1-((2-(trimethylsilyl)ethoxy)methyl)-1H-pyrrolo[3,2-c]pyridin-6-amine (200 mg, 0.47 mmol) in Methanol (2 mL) was added palladium/carbon (497.7 mg, 0.47 mmol) (10% purity) and under nitrogen, then the mixture was stirred at 25° C. for 16 h under hydrogen (15 Psi). LCMS showed 2-(cyclohex-1-en-1-yl)-N-(tetrahydro-2H-pyran-4-yl)-1-((2-(trimethylsilyl)ethoxy)methyl)-1H-pyrrolo[3,2-c]pyridin-6-amine was consumed completely and a peak (95%) with desired mass. The mixture was filtered with a pad of silica gel (100-200 mesh) and Celite, washed with methanol (20 mL), the filtrate was concentrated under vacuum to give 2-cyclohexyl-N-(tetrahydro-2H-pyran-4-yl)-1-((2-(trimethylsilyl) ethoxy)methyl)-1H-pyrrolo[3,2-c]pyridin-6-amine (200 mg, 0.47 mmol, 99.53% yield) as a green oil. MS (ESI): m/z 430.3 [M+1]⁺.

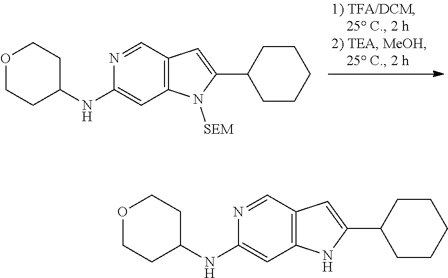 [SMILES: C[Si](C)(C)CCOCn1c(C2CCCCC2)cc2cnc(NC3CCOCC3)cc21 ; c1nc(NC2CCOCC2)cc2[nH]c(C3CCCCC3)cc12]

2-Cyclohexyl-N-(tetrahydro-2H-pyran-4-yl)-1H-pyrrolo[3,2-c]pyridin-6-amine. To a mixture of 2-cyclohexyl-N-(tetrahydro-2H-pyran-4-yl)-1-((2-(trimethylsilyl) ethoxy)methyl)-1H-pyrrolo[3,2-c]pyridin-6-amine (200. mg, 0.47 mmol) in dichloromethane (2 mL) was added Trifluoroacetic acid (2 mL, 0.47 mmol). The mixture was stirred at 25° C. for 2 h. LCMS showed 2-cyclohexyl-N-(tetrahydro-2H-pyran-4-yl)-1-((2-(trimethylsilyl)ethoxy)methyl)-1H-pyrrolo[3,2-c]pyridin-6-amine was consumed completely and a peak (59%) with desired mass. The mixture was concentrated under reduced pressure to give a residue. To the residue in Methanol (2 mL) was added triethylamine (2 mL, 0.47 mmol), the mixture was stirred at 25° C. for 2 h. LCMS showed 2-cyclohexyl-N-(tetrahydro-2H-pyran-4-yl)-1-((2-(trimethylsilyl)ethoxy)methyl)-1H-pyrrolo[3,2-c]pyridin-6-amine was consumed completely and a peak (50%) with desired mass. The residue was purified by prep-HPLC (column: Phenomenex Synergi C18 150*25 mm*10 um; mobile phase: [water(0.1% TFA)-ACN]; B %: 23%-54%, 10 min) followed by lyophilization to give 2-cyclohexyl-N-(tetrahydro-2H-pyran-4-yl)-1H-pyrrolo[3,2-c]pyridin-6-amine (18.09 mg, 0.053 mmol, 11.37% yield) as a off-white solid. ¹H NMR (400 MHz, DMSO-d₆) δ 10.52 (s, 1H), 8.12 (s, 1H), 6.27 (s, 1H), 5.93-5.92 (m, 1H), 5.55 (d, J=8.3 Hz, 1H), 3.87-3.85 (m, 2H), 3.85-3.83 (m, 1H), 3.42-3.37 (m, 2H), 2.63-2.54 (m, 1H), 1.96-1.95 (m, 2H), 1.88-1.85 (m, 2H), 1.78-1.77 (m, 2H), 1.69-1.67 (m, 1H), 1.41-1.36 (m, 6H), 1.25-1.16 (m, 1H), MS (ESI): m/z 300.3 [M+1]⁺.

### Example 27: 1-Methyl-N-(tetrahydro-2H-pyran-4-yl)-2-(6-((2,2,2-trifluoroethyl)amino)pyrimidin-4-yl)-1H-pyrrolo[3,2-c]pyridin-6-amine

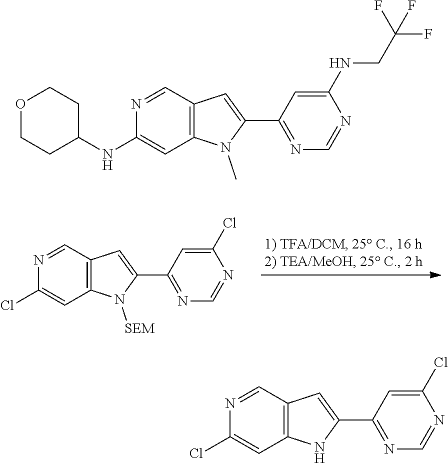

6-Chloro-2-(6-chloropyrimidin-4-yl)-1H-pyrrolo[3,2-c]pyridine. To a solution of 6-chloro-2-(6-chloropyrimidin-4-yl)-1-((2-(trimethylsilyl)ethoxy)methyl)-1H-pyrrolo[3,2-c]pyridine (500 mg, 1.26 mmol) in dichloromethane (10 mL) was added Trifluoroacetic acid (10 mL, 1.26 mmol), the mixture was stirred at 25° C. for 16 h. LCMS showed 6-chloro-2-(6-chloropyrimidin-4-yl)-1-((2-(trimethylsilyl)ethoxy)methyl)-1H-pyrrolo[3,2-c]pyridine was consumed completely and a peak (58%) with desired mass. The mixture was concentrated under oil pump. To the residue in Methanol (10 mL) was added triethylamine (10. mL, 1.26 mmol), then stirred at 25° C. for 2 h. LCMS showed 6-chloro-2-(6-chloropyrimidin-4-yl)-1-((2-(trimethylsilyl)ethoxy)methyl)-1H-pyrrolo[3,2-c]pyridine was consumed completely and a peak (60%) with desired mass. The mixture was concentrated under vacuum. The residue was triturated with methanol (10 mL) and stirred for 2 h. The suspension was filtered and filtered cake was dried under vacuum to 6-chloro-2-(6-chloropyrimidin-4-yl)-1H-pyrrolo[3,2-c]pyridine (300 mg, 1.1316 mmol, 89.482% yield) as a yellow solid. ¹H NMR (400 MHz, DMSO-d₆) δ 9.11 (d, J=1.0 Hz, 1H), 8.79 (d, J=0.6 Hz, 1H), 8.40 (d, J=1.1 Hz, 1H), 7.74 (s, 1H), 7.46 (s, 1H). MS (ESI). m/z 264.8 [M+1]⁺.

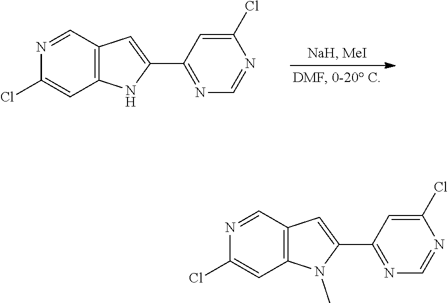 [SMILES: Clc1cc(-c2cc3cnc(Cl)cc3[nH]2)ncn1 ; Cn1c(-c2cc(Cl)ncn2)cc2cnc(Cl)cc21]

6-Chloro-2-(6-chloropyrimidin-4-yl)-1-methyl-1H-pyrrolo[3,2-c]pyridine. To a solution of 6-chloro-2-(6-chloropyrimidin-4-yl)-1H-pyrrolo[3,2-c]pyridine (300. mg, 1.13 mmol) in DMF (5 mL) was added sodium hydride (90.53 mg, 2.26 mmol) at 0° C. in portions, the mixture was stirred at 25° C. for 0.5 h. Then iodomethane (240.94 mg, 1.7 mmol) was added to the mixture at 0° C., the mixture was stirred at 25° C. for 1 h. TLC (Petroleum ether:Ethyl acetate=2:1) indicated starting material was consumed and one major new spot with lower polarity. LCMS showed 6-chloro-2-(6-chloropyrimidin-4-yl)-1H-pyrrolo[3,2-c]pyridine was consumed completely and a peak (62%) with desired mass. The mixture was poured into saturated ammonium chloride aqueous (50 mL). The solid was filtered. The filtered cake was dried under oil pump. LCMS showed the purity was not enough. The residue was purified by flash silica gel chromatography (Biotage, 4 g SepaFlash® Silica Flash Column, Eluent of 10˜30% Ethyl acetate/Petroleum ether gradient @ 20 mL/min) to give 6-chloro-2-(6-chloropyrimidin-4-yl)-1-methyl-1H-pyrrolo[3,2-c]pyridine (150 mg, 0.4439 mmol, 39.226% yield) as a white solid. ¹H NMR (400 MHz, DMSO-d₆) δ 9.13 (s, 1H), 8.76 (s, 1H), 8.30 (s, 1H), 7.80 (s, 1H), 7.62 (s, 1H), 4.10 (s, 3H). MS (ESI): m/z 274.9 [M+1]⁺.

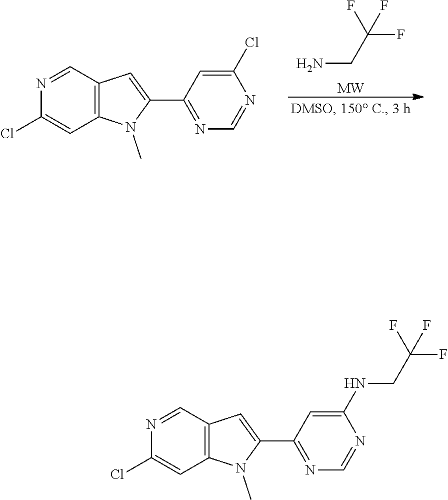

6-(6-chloro-1-methyl-1H-pyrrolo[3,2-c]pyridin-2-yl)-N-(2,2,2-trifluoroethyl)pyrimidin-4-amine. To a solution of 6-chloro-2-(6-chloropyrimidin-4-yl)-1-methyl-1H-pyrrolo[3,2-c]pyridine (150. mg, 0.5400 mmol) in DMSO (3 mL) was added 2,2,2-trifluoroethanamine (0.84 mL, 10.75 mmol) under nitrogen, the mixture was stirred at 150° C. for 2 h under microwave. LCMS showed 6-chloro-2-(6-chloropyrimidin-4-yl)-1-methyl-1H-pyrrolo[3,2-c]pyridine was consumed completely and a peak (78%) with desired mass. The mixture was cooled and poured into water (30 mL). The aqueous phase was extracted with ethyl acetate (20 mL×2). The combined organic phase was washed with brine (10 mL×2), dried over anhydrous sodium sulfate, filtered and concentrated under vacuum. The residue was purified by flash silica gel chromatography (Biotage, 4 g SepaFlash® Silica Flash Column, Eluent of 30˜60% Ethyl acetate/Petroleum ether gradient @ 20 mL/min) to give 6-(6-chloro-1-methyl-1H-pyrrolo[3,2-c]pyridin-2-yl)-N-(2,2,2-trifluoroethyl)pyrimidin-4-amine (110 mg, 0.3219 mmol, 59.899% yield) as a white solid. ¹H NMR (400 MHz, DMSO-d₆) δ 8.72 (s, 1H), 8.65 (s, 1H), 8.18 (s, 1H), 7.75 (s, 1H), 7.15-7.09 (m, 2H), 4.35-4.27 (m, 2H), 4.02 (s, 3H). MS (ESI): m/z 341.9 [M+1]⁺.

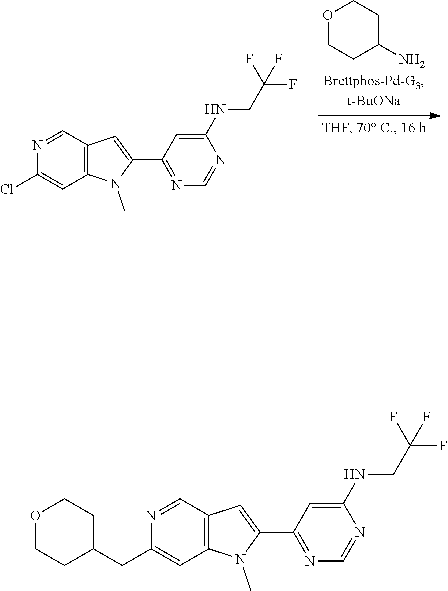 [SMILES: Cn1c(-c2cc(NCC(F)(F)F)ncn2)cc2cnc(Cl)cc21 ; NC1CCOCC1 ; Cn1c(-c2cc(NCC(F)(F)F)ncn2)cc2cnc(CC3CCOCC3)cc21]

1-Methyl-N-(tetrahydro-2H-pyran-4-yl)-2-(6-((2,2,2-trifluoroethyl)amino)pyrimidin-4-yl)-1H-pyrrolo[3,2-c]pyridin-6-amine. To a solution of 6-(6-chloro-1-methyl-1H-pyrrolo[3,2-c]pyridin-2-yl)-N-(2,2,2-trifluoroethyl)pyrimidin-4-amine (110. mg, 0.3200 mmol) and tetrahydropyran-4-amine (97.68 mg, 0.9700 mmol) in THF (3 mL) were added Sodium tert-Butoxide (0.32 mL, 0.6400 mmol) and Methanesulfonato(2-dicyclohexylphosphino-3,6-dimethoxy-2,4,6-tri-i-propyl-1,1-biphenyl)(2-amino-1,1-biphenyl-2-yl)palladium(II) (29.18 mg, 0.0300 mmol) under nitrogen, the mixture was stirred at 80° C. for 16 h. LCMS showed 6-(6-chloro-1-methyl-1H-pyrrolo[3,2-c]pyridin-2-yl)-N-(2,2,2-trifluoroethyl)pyrimidin-4-amine remained (33%) and a peak (36%) with desired mass. Then tetrahydropyran-4-amine (97.68 mg, 0.9700 mmol), Methanesulfonato(2-dicyclohexylphosphino-3,6-dimethoxy-2,4,6-tri-i-propyl-1,1-biphenyl)(2-amino-1,1-biphenyl-2-yl)palladium(II) (29.18 mg, 0.0300 mmol) and Sodium tert-Butoxide (0.32 mL, 0.6400 mmol) was added to the mixture under nitrogen and the mixture was stirred at 80° C. for 6 h. LCMS showed 6-(6-chloro-1-methyl-1H-pyrrolo[3,2-c]pyridin-2-yl)-N-(2,2,2-trifluoroethyl)pyrimidin-4-amine was consumed completely and a peak (40%) with desired mass. The mixture was filtered with a pad of silica gel (100-200 mesh), washed with dichloromethane:methanol=10:1 (100 mL), the filtrate was concentrated under vacuum. The residue was purified by prep-HPLC (column: Phenomenex Luna C18 150*25 mm*l0 um; mobile phase: [water (0.225% FA) -ACN]; B %: 9-39%, 10 min), then dried by lyophilization to give 1-methyl-N-tetrahydropyran-4-yl-2-[6-(2,2,2-trifluoroethylamino)pyrimidin-4-yl]pyrrolo[3,2-c]pyridin-6-amine (68.44 mg, 0.1664 mmol, 51.688% yield) as a yellow solid. ¹H NMR (400 MHz, MeOD) δ 8.61 (d, J=1.1 Hz, 1H), 8.38-8.37 (m, 2H), 6.98-6.96 (m, 2H), 6.70 (s, 1H), 4.26 (q, J=9.2 Hz, 2H), 4.02-4.00 (m, 2H), 3.90 (s, 3H), 3.89-3.82 (m, 1H), 3.63-3.60 (m, 2H), 2.07-2.03 (m, 2H), 1.66-1.59 (m, 2H); MS (ESI): m/z 407.1 [M+1]⁺.

### Example 28: (R)—N-(Tetrahydrofuran-3-yl)-2-(2-((2,2,2-trifluoroethyl)amino)pyrimidin-4-yl)-1H-pyrrolo[3,2-c]pyridin-6-amine

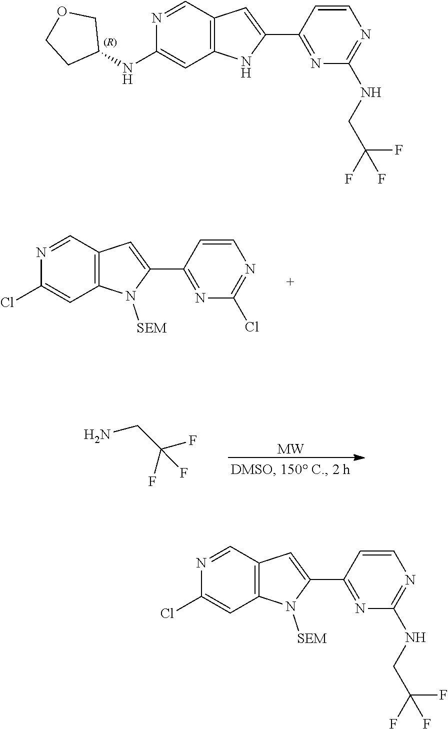

4-(6-Chloro-1-((2-(trimethylsilyl)ethoxy)methyl)-1H-pyrrolo[3,2-c]pyridin-2-yl)-N-(2,2,2-trifluoroethyl)pyrimidin-2-amine. To a solution of 6-chloro-2-(2-chloropyrimidin-4-yl)-1-((2-(trimethylsilyl)ethoxy)methyl)-1H-pyrrolo[3,2-c]pyridine (800. mg, 2.02 mmol) in DMSO (4 mL) was added 2,2,2-trifluoroethanamine (3.18 mL, 40.47 mmol) under nitrogen, the mixture was stirred at 150° C. for 2 h under microwave. LCMS showed 6-chloro-2-(2-chloropyrimidin-4-yl)-1-((2-(trimethylsilyl)ethoxy)methyl)-1H-pyrrolo[3,2-c]pyridine was consumed completely and a peak (44%) with desired mass. The mixture was poured into water (50 mL). The aqueous phase was extracted with ethyl acetate (40 mL×3). The combined organic phase was washed with brine (40 mL×5), dried over anhydrous sodium sulfate, filtered and concentrated under vacuum. The residue was purified by silica gel chromatography (200-300 mesh silica gel, Petroleum ether/Ethyl acetate=100/1, 3/1, Petroleum ether/Ethyl acetate=1/1, Rf=0.6) to give 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine (650 mg, 1.0503 mmol, 51.907% yield) as a yellow solid. MS (ESI): m/z 458.2 [M+1]⁺.

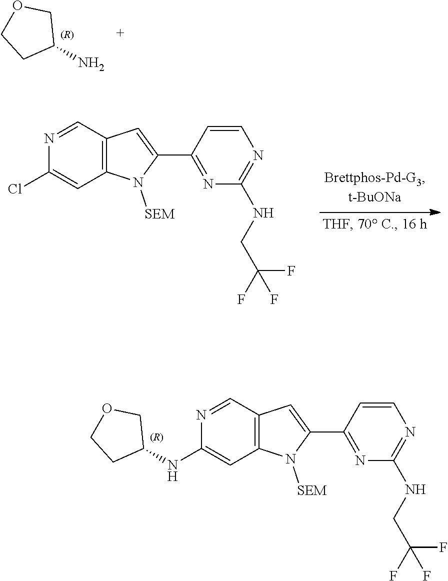

(R)—N-(Tetrahydrofuran-3-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine. To a solution of 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine (100. mg, 0.2200 mmol) in THF (2 mL) was added sodium tert-butoxide (0.22 mL, 0.4400 mmol, 2M in THF), methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (14. mg, 0.0200 mmol) and rac-(3R)-tetrahydrofuran-3-amine (57.07 mg, 0.6600 mmol), the reaction mixture as stirred at 70° C. for 16 h under nitrogen. LCMS showed 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine was consumed completely and a peak (61%) with desired mass. The mixture was cooled and filtered with a pad of Celite, the filtrate was concentrated under vacuum to give a residue. The residue was purified by silica gel chromatography (200-300 mesh silica gel, Petroleum ether/Ethyl acetate=100/1, 0/1, Petroleum ether/Ethyl acetate=0/1, Rf=0.3) to give (R)—N-(tetrahydrofuran-3-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine (80 mg, 0.1537 mmol, 70.375% yield) as yellow solid. MS (ESI): m/z 509.4 [M+1]⁺.

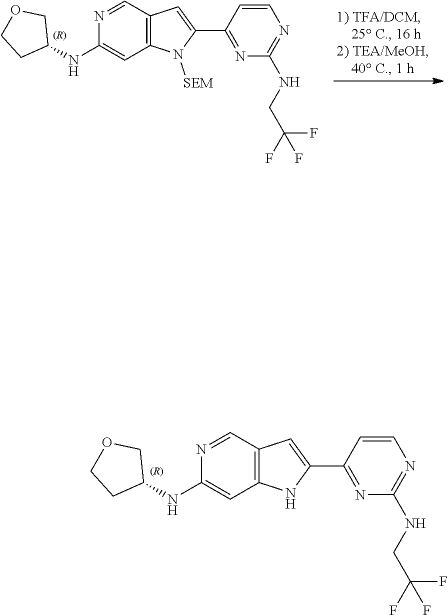

(R)—N-(Tetrahydrofuran-3-yl)-2-(2-((2,2,2-trifluoroethyl)amino)pyrimidin-4-yl)-1H-pyrrolo[3,2-c]pyridin-6-amine. To a solution of (R)—N-(tetrahydrofuran-3-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine (80. mg, 0.1600 mmol) in dichloromethane (2 mL) was added trifluoroacetic acid (2. mL, 26.12 mmol), the reaction mixture was stirred at 25° C. for 16 h. LCMS showed (R)—N-(tetrahydrofuran-3-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine was consumed completely, the mixture was concentrated under reduced pressure. To the residue in methanol (2 mL) was added triethylamine (2. mL, 14.35 mmol), the reaction mixture was stirred at 40° C. for 1 h. LCMS showed a peak (87%) with desired mass. The reaction mixture was concentrated in reduced pressure. The residue was purified by prep-HPLC (column Phenomenex luna C18 150*25 mm*10 um; mobile phase: [water(0.225% FA)-ACN]; B %: 4%-34%, 9 min) followed by lyophilization to give (R)—N-(tetrahydrofuran-3-yl)-2-(2-((2,2,2-trifluoroethyl)amino)pyrimidin-4-yl)-1H-pyrrolo[3,2-c]pyridin-6-amine (30.46 mg, 0.0774 mmol, 49.187% yield, formic acid) as a yellow solid. ¹H NMR (400 MHz, DMSO-d₆) δ 11.71-11.65 (m, 1H), 8.54 (s, 1H), 8.40 (d, J=4.9 Hz, 1H), 7.79-7.74 (m, 1H), 7.35 (s, 1H), 7.27 (d, J=5.1 Hz, 1H), 7.05-7.02 (m, 1H), 6.59 (s, 1H), 4.40-4.30 (m, 3H), 3.92-3.87 (m, 2H), 3.77-3.76 (m, 1H), 3.66-3.65 (m, 1H), 2.22-2.22 (m, 1H), 1.88-1.87 (m, 1H). MS (ESI): m/z 379.2 [M+1]⁺.

### Example 29: 2-(2-(Methyl(2,2,2-trifluoroethyl)amino)pyrimidin-4-yl)-N-(tetrahydro-2H-pyran-4-yl)-1h-pyrrolo[3,2-c]pyridin-6-amine

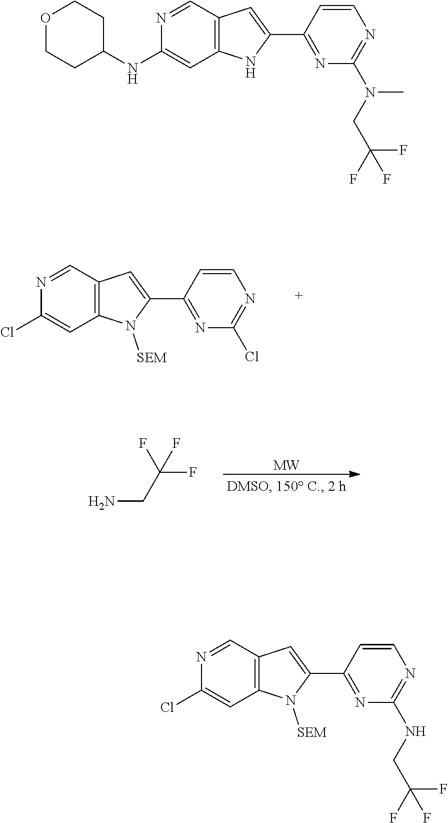

4-(6-Chloro-1-((2-(trimethylsilyl)ethoxy)methyl)-1H-pyrrolo[3,2-c]pyridin-2-yl)-N-(2,2,2-trifluoroethyl)pyrimidin-2-amine. To a solution of 6-chloro-2-(2-chloropyrimidin-4-yl)-1-((2-(trimethylsilyl)ethoxy)methyl)-1H-pyrrolo[3,2-c]pyridine (1000 mg, 2.53 mmol) in DMSO (3 mL) was added 2,2,2-trifluoroethanamine (7516.69 mg, 75.88 mmol), the mixture was stirred at 150° C. for 2 h under microwave. LCMS showed 6-chloro-2-(2-chloropyrimidin-4-yl)-1-((2-(trimethylsilyl)ethoxy)methyl)-1H-pyrrolo[3,2-c]pyridine was consumed completely and a peak (66%) with desired mass. The mixture was cooled and poured into water (40 mL). The aqueous phase was extracted with ethyl acetate (30 mL×3). The combined organic phase was washed with brine (15 mL×2), dried over anhydrous sodium sulfate, filtered and concentrated under vacuum. The residue was purified by flash silica gel chromatography (Biotage, 24 g SepaFlash® Silica Flash Column, Eluent of 0˜33% Ethyl acetate/Petroleum ether gradient @ 45 mL/min) to give 4-(6-chloro-1-((2-(trimethylsilyl)ethoxy)methyl) -1H-pyrrolo[3,2-c]pyridin-2-yl)-N-(2,2,2-trifluoroethyl)pyrimidin-2-amine (770 mg, 1.68 mmol, 66.4% yield) as a white solid. ¹H NMR (400 MHz, DMSO-d₆) δ 8.76 (s, 1H), 8.47 (d, J=5.1 Hz, 1H), 8.02 (t, J=6.7 Hz, 1H), 7.90 (s, 1H), 7.45 (s, 1H), 7.29 (d, J=5.1 Hz, 1H), 6.32-6.12 (m, 2H), 4.25-4.16 (m, 2H), 3.38 (d, J=8.0 Hz, 2H), 0.69-0.57 (m, 2H), -0.24 (s, 9H), MS (ESI): m/z 458.2 [M+1]⁺.

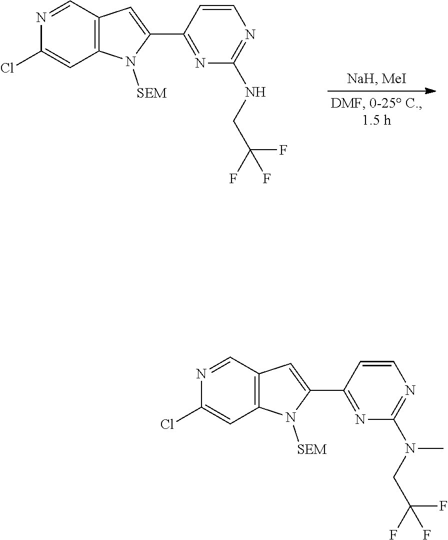

4-(6-Chloro-1-((2-(trimethylsilyl)ethoxy)methyl)-1H-pyrrolo[3,2-c]pyridin-2-yl)-N-methyl-N-(2,2,2-trifluoroethyl)pyrimidin-2-amine. To a mixture of 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine (120. mg, 0.26 mmol) in DMF (4 mL) was added Sodium Hydride (15.72 mg, 0.39 mmol) at 0° C., the mixture was stirred at 0° C. for 0.5 h. Then methyl iodide (55.79 mg, 0.39 mmol) was added in the mixture at 0° C. The mixture was stirred at 25° C. for 1 h. TLC (Petroleum ether:Ethyl acetate=2:1) indicated starting material was consumed and one major new spot with lower polarity. The residue was poured into ammonium chloride (30 mL). The aqueous phase was extracted with ethyl acetate (30 mL×3). The combined organic phase was washed with brine (20 mL×2), dried over anhydrous sodium sulfate, filtered and concentrated under vacuum. The residue was purified by silica gel chromatography (100-200 mesh silica gel, Petroleum ether/Ethyl acetate=0/1, 3/1) to give 4-(6-chloro-1-((2-(trimethylsilyl)ethoxy)methyl)-1H-pyrrolo[3,2-c]pyridin-2-yl)-N-methyl-N-(2,2,2-trifluoroethyl)pyrimidin-2-amine (110 mg, 0.23 mmol, 88.94% yield) as a yellow oil. MS (ESI): m/z 472.3 [M+1]⁺.

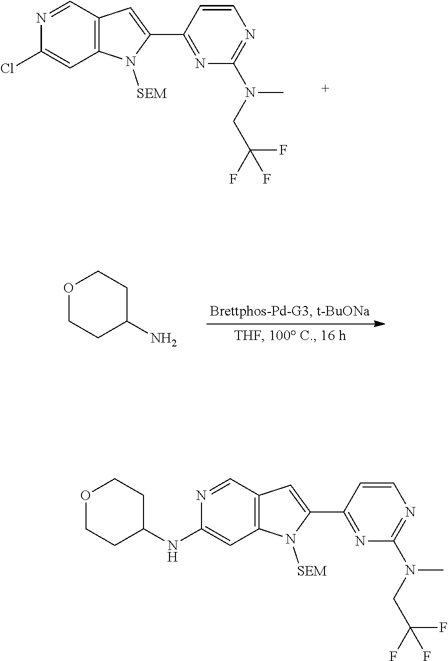 [SMILES: CN(CC(F)(F)F)c1nccc(-c2cc3cnc(Cl)cc3n2COCC[Si](C)(C)C)n1 ; NC1CCOCC1 ; CN(CC(F)(F)F)c1nccc(-c2cc3cnc(NC4CCOCC4)cc3n2COCC[Si](C)(C)C)n1]

2-(2-(Methyl(2,2,2-trifluoroethyl)amino)pyrimidin-4-yl)-N-(tetrahydro-2H-pyran-4-yl)-1-((2-(trimethylsilyl)ethoxy)methyl)-1H-pyrrolo[3,2-c]pyridin-6-amine. To a mixture of 4-(6-chloro-1-((2-(trimethylsilyl)ethoxy)methyl)-1H-pyrrolo[3,2-c]pyridin-2-yl)-N-methyl-N-(2,2,2-trifluoroethyl)pyrimidin-2-amine (110. mg, 0.23 mmol) and tetrahydro-2H-pyran-4-amine (47.15 mg, 0.47 mmol) in THF (1 mL) was added Methanesulfonato(2-dicyclohexylphosphino-3,6-dimethoxy-2,4,6-tri-I-propyl-1,1-biphenyl)(2-amino-1,1-biphenyl-2-yl)palladium(II) (21.13 mg, 0.02 mmol) and Sodium tert-Butoxide (0.47 mL, 0.47 mmol). The mixture was stirred at 70° C. for 16 h under nitrogen atmosphere. LCMS showed 4-[6-chloro-1-(2-trimethylsilylethoxymethyl)pyrrolo[3,2-c]pyridin-2-yl]-N-methyl-N-(2,2,2-trifluoroethyl)pyrimidin-2-amine was consumed completely and a peak (76%) with desired mass. The mixture was concentrated under reduced pressure to give a residue. The residue was purified by flash silica gel chromatography (Biotage, 24 g SepaFlash® Silica Flash Column, Eluent of 0˜40% Ethyl acetate/Petroleum ether gradient @ 30 mL/min) to give 2-(2-(methyl(2,2,2-trifluoroethyl)amino)pyrimidin-4-yl)-N-(tetrahydro-2H-pyran-4-yl)-1-((2-(trimethylsilyl)ethoxy)methyl)-1H-pyrrolo[3,2-c]pyridin-6-amine (120 mg, 0.22 mmol, 95.94% yield) as a yellow oil. MS (ESI): m/z 537.3 [M+1]⁺.

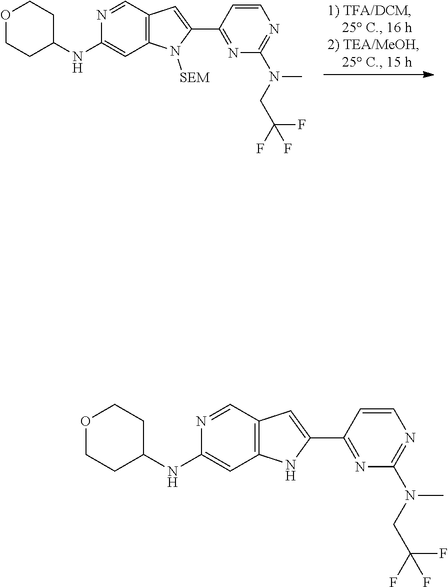

2-(2-(Methyl(2,2,2-trifluoroethyl)amino)pyrimidin-4-yl)-N-(tetrahydro-2H-pyran-4-yl)-1H-pyrrolo[3,2-c]pyridin-6-amine. To a mixture of 2-(2-(methyl(2,2,2-trifluoroethyl)amino)pyrimidin-4-yl)-N-(tetrahydro-2H-pyran-4-yl)-1-((2-(trimethylsilyl)ethoxy)methyl)-1H-pyrrolo[3,2-c]pyridin-6-amine (120 mg, 0.22 mmol) in DCM (2 mL) was added Trifluoroacetic acid (2 mL, 0.22 mmol). The mixture was stirred at 25° C. for 16 h. LCMS showed 2-(2-(methyl(2,2,2-trifluoroethyl)amino)pyrimidin-4-yl)-N-(tetrahydro-2H-pyran-4-yl)-1-((2-(trimethylsilyl)ethoxy)methyl)-1H-pyrrolo[3,2-c]pyridin-6-amine was consumed completely and a peak (92%) with the mass of intermediate. The mixture was concentrated under reduced pressure to give a residue. Then to the residue in Methanol (2 mL) was added triethylamine (2 mL, 0.22 mmol), the mixture was stirred at 25° C. for 1 h. LCMS showed a peak (95%) with desired mass. The mixture was concentrated under reduced pressure to give a residue. The residue was purified by prep-HPLC (column: Unisil 3-100 C18 Ultra 150*50 mm*3 um; mobile phase: [water (0.225% FA)-ACN]; B %: 13%-43%, 10 min) followed by lyophilization to give 2-(2-(methyl(2,2,2-trifluoroethyl)amino)pyrimidin-4-yl)-N-(tetrahydro-2H-pyran-4-yl)-1H-pyrrolo[3,2-c]pyridin-6-amine (38.01 mg, 0.09 mmol, 40.45% yield, formic acid) as a yellow solid. ¹H NMR (400 MHz, DMSO-d₆) δ 11.46 (s, 1H), 8.48 (s, 1H), 8.42 (d, J=5.3 Hz, 1H), 8.13 (s, 0.6H), 7.32 (s, 1H), 7.27 (d, J=5.1 Hz, 1H), 6.61-6.45 (m, 2H), 4.88-4.54 (m, 2H), 3.94-3.86 (m, 2H), 3.85-3.75 (m, 1H), 3.45-3.39 (m, 2H), 3.31-3.23 (m, 3H), 1.93-1.90 (m, 2H), 1.55-1.38 (m, 2H); MS (ESI): m/z 407.2 [M+1]⁺.

### Example 30: 1-Methyl-2-(2-(methyl(2,2,2-trifluoroethyl)amino)pyrimidin-4-yl)-N-(tetrahydro-2h-pyran-4-yl)-1H-pyrrolo[3,2-c]pyridin-6-amine

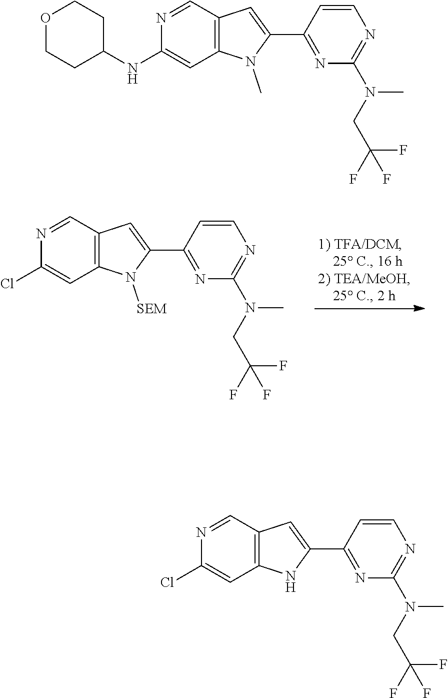

N-(1-Methyl-2-(6-methylpyrimidin-4-yl)-1H-pyrrolo[3,2-c]pyridin-6-yl)acetamide. To a mixture of 4-(6-chloro-1-((2-(trimethylsilyl)ethoxy)methyl)-1H-pyrrolo[3,2-c]pyridin-2-yl)-N-methyl-N-(2,2,2-trifluoroethyl)pyrimidin-2-amine (270 mg, 0.57 mmol) in DCM (2 mL) was added Trifluoroacetic acid (2 mL, 0.57 mmol). The mixture was stirred at 25° C. for 16 h. LCMS showed 4-(6-chloro-1-((2-(trimethylsilyl)ethoxy)methyl)-1H-pyrrolo[3,2-c]pyridin-2-yl)-N-methyl-N-(2,2,2-trifluoroethyl)pyrimidin-2-amine was consumed completely and a peak (94%) with the mass of intermediate. The mixture was concentrated under reduced pressure to give a residue. Then the residue in Methanol (2 mL) was added triethylamine (2 mL, 0.57 mmol), the mixture was stirred at 25° C. for 1 h. LCMS showed a peak (94%) with desired mass. The mixture was concentrated under reduced pressure to give a residue. The residue was purified by silica gel chromatography (100-200 mesh silica gel, Petroleum ether/Ethyl acetate=0/1, 1/0) to give 4-(6-chloro-1H-pyrrolo[3,2-c]pyridin-2-yl)-N-methyl-N-(2,2,2-trifluoroethyl)pyrimidin-2-amine (190 mg, 0.57 mmol, 97.20% yield) as a yellow oil. MS (ESI): m/z 342.2 [M+1]⁺.

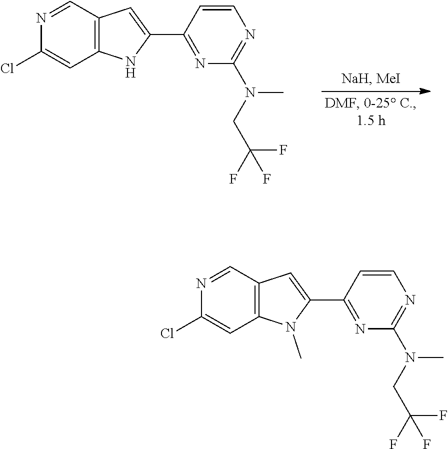 [SMILES: CN(CC(F)(F)F)c1nccc(-c2cc3cnc(Cl)cc3[nH]2)n1 ; CN(CC(F)(F)F)c1nccc(-c2cc3cnc(Cl)cc3n2C)n1]

4-(6-Chloro-1-methyl-1H-pyrrolo[3,2-c]pyridin-2-yl)-N-methyl-N-(2,2,2-trifluoroethyl)pyrimidin-2-amine. To a mixture of 4-(6-chloro-1H-pyrrolo[3,2-c]pyridin-2-yl)-N-methyl-N-(2,2,2-trifluoroethyl)pyrimidin-2-amine (190 mg, 0.56 mmol) in DMF (2 mL) was added Sodium Hydride (33 mg, 0.83 mmol) at 0° C., the mixture was stirred at 25° C. for 0.5 h. Then methyl iodide (0.05 mL, 0.83 mmol) was added in the mixture at 0° C., the mixture was stirred at 25° C. for 1 h. TLC (Petroleum ether:Ethyl acetate=1:1) indicated starting material was consumed and one major new spot with lower polarity. The residue was poured into ammonium chloride (30 mL). The aqueous phase was extracted with ethyl acetate (20 mL×3). The combined organic phase was washed with brine (10 mL×2), dried over anhydrous sodium sulfate, filtered and concentrated under vacuum. The residue was purified by silica gel chromatography (100-200 mesh silica gel, Petroleum ether/Ethyl acetate=0/1, 2/1) to give 4-(6-chloro-1-methyl-1H-pyrrolo[3,2-c]pyridin-2-yl)-N-methyl-N-(2,2,2-trifluoroethyl)pyrimidin-2-amine (180 mg, 0.51 mmol, 91.0% yield) as a yellow oil. MS (ESI): m/z 356.1 [M+1]⁺.

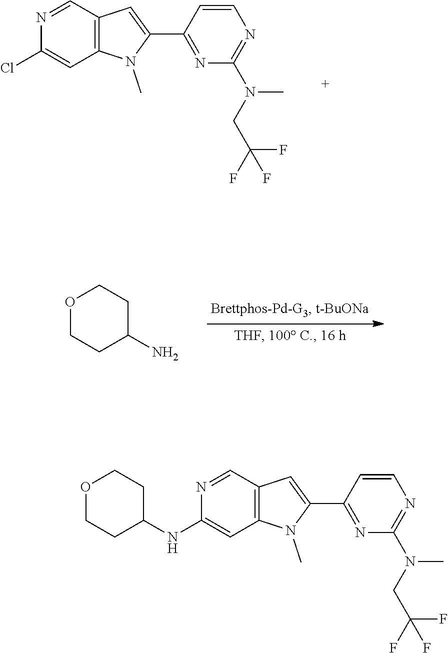 [SMILES: CN(CC(F)(F)F)c1nccc(-c2cc3cnc(Cl)cc3n2C)n1 ; NC1CCOCC1 ; CN(CC(F)(F)F)c1nccc(-c2cc3cnc(NC4CCOCC4)cc3n2C)n1]

1-Methyl-2-(2-(methyl(2,2,2-trifluoroethyl)amino)pyrimidin-4-yl)-N-(tetrahydro-2H-pyran-4-yl)-1H-pyrrolo[3,2-c]pyridin-6-amine. To a mixture of 4-(6-chloro-1-methyl-1H-pyrrolo[3,2-c]pyridin-2-yl)-N-methyl-N-(2,2,2-trifluoroethyl)pyrimidin-2-amine (80 mg, 0.22 mmol) and tetrahydro-2H-pyran-4-amine (68 mg, 0.67 mmol) in THF (1 mL) was added methanesulfonato(2-dicyclohexylphosphino-3,6-dimethoxy-2,4,6-tri-i-propyl-1,1-biphenyl)(2-amino-1,1-biphenyl-2-yl)palladium(II) (20.39 mg, 0.02 mmol) and Sodium tert-Butoxide (0.22 mL, 0.45 mmol). The mixture was stirred at 70° C. for 16 h. LCMS showed 4-(6-chloro-1-methyl-1H-pyrrolo[3,2-c]pyridin-2-yl)-N-methyl-N-(2,2,2-trifluoroethyl)pyrimidin-2-amine was consumed completely and a peak (71%) with desired mass. The residue was purified by silica gel chromatography (100-200 mesh silica gel, Petroleum ether/Ethyl acetate=0/1, 1/0) followed by prep-HPLC (column: Unisil 3-100 C18 Ultra 150*50 mm*3 um; mobile phase: [water (0.225% FA)-ACN]; B %: 15%-45%, 10 min) followed by lyophilization to give 1-methyl-2-(2-(methyl(2,2,2-trifluoroethyl)amino)pyrimidin-4-yl)-N-(tetrahydro-2H-pyran-4-yl)-1H-pyrrolo[3,2-c]pyridin-6-amine (45.61 mg, 0.11 mmol, 47.7% yield, formic acid) as a yellow solid. ¹H NMR (400 MHz, DMSO-d₆) δ=8.44-8.38 (m, 2H), 8.15 (s, 1H), 7.27-7.18 (m, 2H), 6.39 (s, 1H), 6.12 (d, J=7.9 Hz, 1H), 4.57 (q, J=9.5 Hz, 2H), 4.04-3.93 (m, 3H), 3.91-3.81 (m, 3H), 3.45-3.42 (m, 2H), 3.25 (s, 3H), 1.92-1.88 (m, 2H), 1.53-1.38 (m, 2H); MS (ESI): m/z 421.2 [M+1]⁺.

### Example 31: 2-(2-(Pyrrolidin-1-yl)pyrimidin-4-yl)-N-(tetrahydro-2H-pyran-4-yl)-1H-pyrrolo[3,2-c]pyridin-6-amine

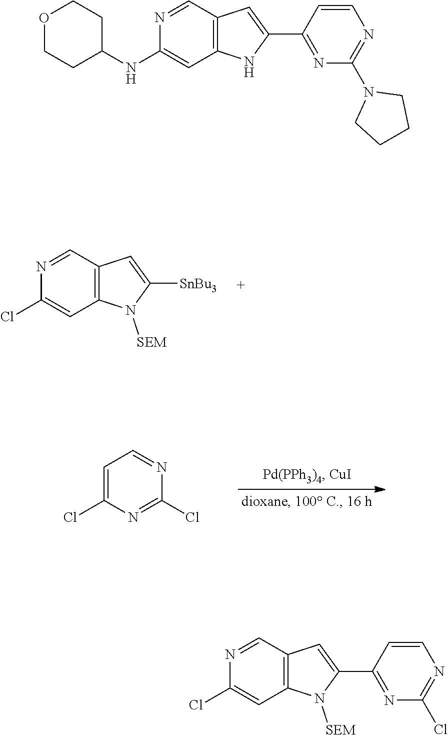

6-Chloro-2-(2-chloropyrimidin-4-yl)-1-((2-(trimethylsilyl)ethoxy)methyl)-1H-pyrrolo[3,2-c]pyridine. To a solution of 6-chloro-2-(tributylstannyl)-1-((2-(trimethylsilyl)ethoxy)methyl)-1H-pyrrolo[3,2-c]pyridine (5. g, 8.74 mmol) and 2,4-dichloropyrimidine (1302.52 mg, 8.74 mmol) in 1,4-Dioxane (40 mL) was added cuprous iodide (333.02 mg, 1.75 mmol) and tetrakis[triphenylphosphine]palladium (1010.3 mg, 0.8700 mmol), the reaction mixture was stirred at 100° C. for 16 h under nitrogen. LCMS showed 6-chloro-2-(tributylstannyl)-1-((2-(trimethylsilyl)ethoxy)methyl)-1H-pyrrolo[3,2-c]pyridine was consumed completely and a peak (42%) with desired mass. The residue was purified by silica gel chromatography (200-300 mesh silica gel, Petroleum ether/Ethyl acetate=100/1, 3/1, Petroleum ether/Ethyl acetate=3/1, Rf=0.3) to give 6-chloro-2-(2-chloropyrimidin-4-yl)-1-((2-(trimethylsilyl)ethoxy)methyl)-1H-pyrrolo[3,2-c]pyridine (1.8 g, 3.6423 mmol, 41.659% yield) as a yellow solid. ¹H NMR (400 MHz, DMSO-d₆) δ 9.07 (d, J=5.4 Hz, 1H), 9.03-9.02 (m, 1H), 8.37 (d, J=5.3 Hz, 1H), 8.12 (s, 1H), 7.92 (d, J=0.6 Hz, 1H), 6.32 (s, 2H), 3.68-3.59 (m, 2H), 0.96-0.92 (m, 2H), 0.00 (s, 9H). MS (ESI): m/z 395.9 [M+1]⁺.

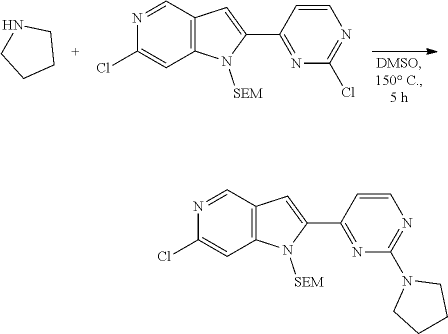 [SMILES: C1CCNC1 ; C[Si](C)(C)CCOCn1c(-c2ccnc(Cl)n2)cc2cnc(Cl)cc21 ; C[Si](C)(C)CCOCn1c(-c2ccnc(N3CCCC3)n2)cc2cnc(Cl)cc21]

6-Chloro-2-(2-(pyrrolidin-1-yl)pyrimidin-4-yl)-1-((2-(trimethylsilyl)ethoxy)methyl)-1H-pyrrolo[3,2-c]pyridine. To a solution of 6-chloro-2-(2-chloropyrimidin-4-yl)-1-((2-(trimethylsilyl)ethoxy)methyl)-1H-pyrrolo[3,2-c]pyridine (250. mg, 0.6300 mmol) in DMSO (2 mL) was added pyrrolidine (500. mg, 7.03 mmol), the reaction mixture was stirred at 150° C. for 5 h under nitrogen. LCMS showed a peak (58%) with desired mass. The mixture was poured into water (50 mL). The aqueous phase was extracted with ethyl acetate (30 mL×3). The combined organic phase was washed with brine (40 mL×6), dried over anhydrous sodium sulfate, filtered and concentrated under vacuum. The residue was purified by silica gel chromatography (200-300 mesh silica gel, Petroleum ether/Ethyl acetate=100/1, 3/1, Petroleum ether/Ethyl acetate=1/1, Rf=0.6) to give 6-chloro-2-(2-(pyrrolidin-1-yl)pyrimidin-4-yl)-1-((2-(trimethylsilyl)ethoxy)methyl)-1H-pyrrolo[3,2-c]pyridine (150 mg, 0.3174 mmol, 50.199% yield) as a yellow oil. MS (ESI): m/z 430.4 [M+1]⁺.

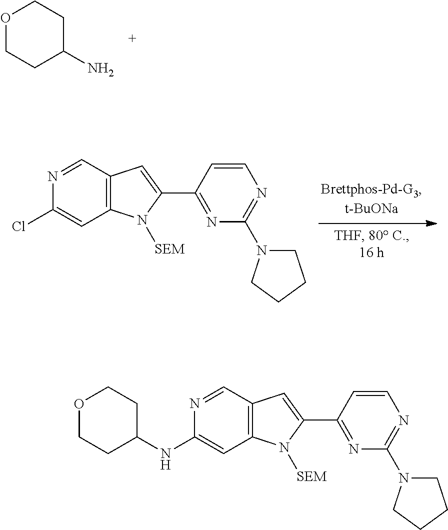 [SMILES: NC1CCOCC1 ; C[Si](C)(C)CCOCn1c(-c2ccnc(N3CCCC3)n2)cc2cnc(Cl)cc21 ; C[Si](C)(C)CCOCn1c(-c2ccnc(N3CCCC3)n2)cc2cnc(NC3CCOCC3)cc21]

2-(2-(Pyrrolidin-1-yl)pyrimidin-4-yl)-N-(tetrahydro-2H-pyran-4-yl)-1-((2-(trimethylsilyl)ethoxy)methyl)-1H-pyrrolo[3,2-c]pyridin-6-amine. To a solution of 6-chloro-2-(2-(pyrrolidin-1-yl)pyrimidin-4-yl)-1-((2-(trimethylsilyl)ethoxy)methyl)-1H-pyrrolo[3,2-c]pyridine (150. mg, 0.3500 mmol) and tetrahydropyran-4-amine (105.85 mg, 1.05 mmol) in THF (1 mL) was added sodium tert-butoxide (0.35 mL, 0.7000 mmol, 2M in THF) and methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (29.8 mg, 0.0300 mmol), the reaction mixture was stirred at 80° C. for 16 h under nitrogen. LCMS showed 6-chloro-2-(2-(pyrrolidin-1-yl)pyrimidin-4-yl)-1-((2-(trimethylsilyl)ethoxy)methyl)-1H-pyrrolo[3,2-c]pyridine was consumed completely and a peak (76%) with desired mass. The mixture was cooled and filtered with a pad of Celite, the filtrate was concentrated under vacuum to give a residue. The residue was purified by silica gel chromatography (200-300 mesh silica gel, Petroleum ether/Ethyl acetate=100/1, 1/1, Petroleum ether/Ethyl acetate=1/1, Rf=0.4) to give 2-(2-(pyrrolidin-1-yl)pyrimidin-4-yl)-N-(tetrahydro-2H-pyran-4-yl)-1-((2-(trimethylsilyl)ethoxy)methyl)-1H-pyrrolo[3,2-c]pyridin-6-amine (150 mg, 0.2987 mmol, 85.622% yield) as a yellow oil. MS (ESI): m/z 495.4 [M+1]⁺.

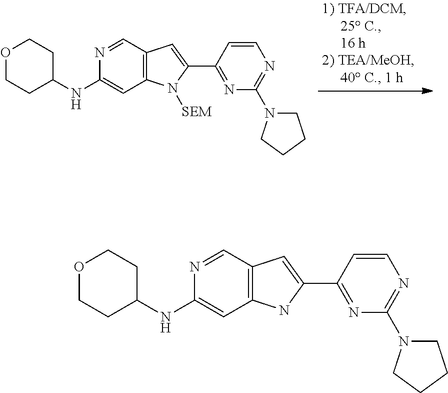 [SMILES: C[Si](C)(C)CCOCn1c(-c2ccnc(N3CCCC3)n2)cc2cnc(NC3CCOCC3)cc21 ; c1cc(-c2cc3cnc(NC4CCOCC4)cc3[nH]2)nc(N2CCCC2)n1]

2-(2-(Pyrrolidin-1-yl)pyrimidin-4-yl)-N-(tetrahydro-2H-pyran-4-yl)-1H-pyrrolo[3,2-c]pyridin-6-amine. To a solution of 2-(2-(pyrrolidin-1-yl)pyrimidin-4-yl)-N-(tetrahydro-2H-pyran-4-yl)-1-((2-(trimethylsilyl)ethoxy)methyl)-1H-pyrrolo[3,2-c]pyridin-6-amine (150. mg, 0.3000 mmol) in dichloromethane (2 mL) was added trifluoroacetic acid (2. mL, 14.35 mmol), the reaction mixture was stirred at 25° C. for 16 h. LCMS showed 2-(2-(pyrrolidin-1-yl)pyrimidin-4-yl)-N-(tetrahydro-2H-pyran-4-yl)-1-((2-(trimethylsilyl)ethoxy)methyl)-1H-pyrrolo[3,2-c]pyridin-6-amine was consumed completely and a peak (93%) with desired mass. the reaction mixture was concentrated under reduced pressure, the residue in methanol (2 mL) was added triethylamine (2. mL, 14.35 mmol), the reaction mixture was stirred at 40° C. for 1 h. LCMS showed a peak (67%) with desired mass. The mixture was concentrated under reduced pressure. The residue was purified by prep-HPLC (column Phenomenex luna C18 150*25 mm*10 um; mobile phase: [water (0.225% FA)-ACN]; B %: 8%-28%, 10 min) followed by lyophilization to give 2-(2-(pyrrolidin-1-yl)pyrimidin-4-yl)-N-(tetrahydro-2H-pyran-4-yl)-1H-pyrrolo[3,2-c]pyridin-6-amine (85.91 mg, 0.23 mmol, 76.1% yield, formic acid) as a yellow solid. ¹H NMR (400 MHz, DMSO-d₆) δ 11.53 (s, 1H), 8.61 (s, 1H), 8.48 (d, J=5.1 Hz, 1H), 7.40 (d, J=1.0 Hz, 1H), 7.23 (d, J=5.1 Hz, 1H), 6.84-6.73 (m, 2H), 4.06-4.03 (m, 2H), 3.94-3.93 (m, 1H), 3.59-3.57 (m, 4H), 3.59-3.56 (m, 2H), 2.15-2.04 (m, 6H), 1.69-1.54 (m, 2H). MS (ESI): m/z 365.3 [M+1]⁺.

### Example 32: N-Cyclobutyl-2-(2-((2,2,2-trifluoroethyl)amino)pyrimidin-4-yl)-1H-pyrrolo[3,2-c]pyridin-6-amine

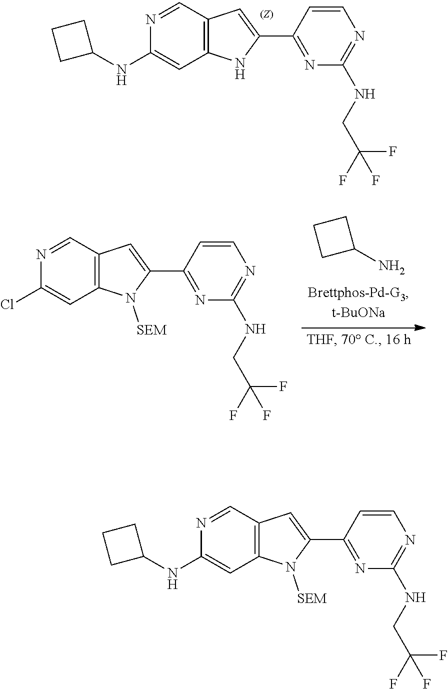

N-Cyclobutyl-2-(2-((2,2,2-trifluoroethyl)amino)pyrimidin-4-yl)-1-((2-(trimethylsilyl) ethoxy)methyl)-1H-pyrrolo[3,2-c]pyridin-6-amine. To a solution of 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine (100 mg, 0.22 mmol) in THE (2 mL) was added sodium tert-butoxide (0.22 mL, 0.44 mmol, 2 M in THF), methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (19.79 mg, 0.0200 mmol) and cyclobutanamine (46 mg, 0.66 mmol), the reaction mixture was stirred at 70° C. for 16 h under nitrogen. LCMS showed 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine was consumed completely and a peak (55%) with desired mass. The mixture was cooled and filtered with a pad of celite, the filtrate was concentrated under vacuum to give a residue, which was purified by silica gel chromatography (200-300 mesh silica gel, Petroleum ether/Ethyl acetate=100/1, 0/1, Petroleum ether/Ethyl acetate=0/1, RF=0.4) to give N-cyclobutyl-2-(2-((2,2,2-trifluoroethyl)amino)pyrimidin-4-yl)-1-((2-(trimethylsilyl)ethoxy)methyl)-1H-pyrrolo[3,2-c]pyridin-6-amine (80 mg, 0.16 mmol, 72.2% yield) as a yellow solid. MS (ESI): m/z 493.1 [M+1]⁺.

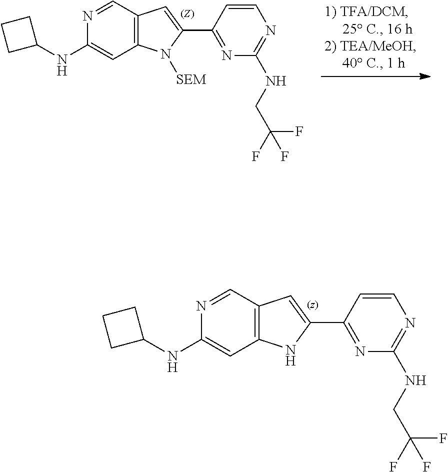 [SMILES: C[Si](C)(C)CCOCn1c(-c2ccnc(NCC(F)(F)F)n2)cc2cnc(NC3CCC3)cc21 ; FC(F)(F)CNc1nccc(-c2cc3cnc(NC4CCC4)cc3[nH]2)n1]

N-Cyclobutyl-2-(2-((2,2,2-trifluoroethyl)amino)pyrimidin-4-yl)-1H-pyrrolo[3,2-c]pyridin-6-amine. To a solution of N-cyclobutyl-2-(2-((2,2,2-trifluoroethyl)amino)pyrimidin-4-yl)-1-((2-(trimethylsilyl)ethoxy)methyl)-1H-pyrrolo[3,2-c]pyridin-6-amine (80. mg, 0.1600 mmol) in dichloromethane (2 mL) was added trifluoroacetic acid (2 mL, 26.31 mmol), the reaction mixture was stirred at 25° C. for 16 h. LCMS showed N-cyclobutyl-2-(2-((2,2,2-trifluoroethyl)amino)pyrimidin-4-yl)-1-((2-(trimethylsilyl)ethoxy)methyl)-1H-pyrrolo[3,2-c]pyridin-6-amine was consumed completely. The mixture was concentrated under reduced pressure. To the residue in methanol (2 mL) was added triethylamine (2. mL, 14.35 mmol), the reaction mixture was stirred at 40° C. for 1 h. LCMS showed a peak (88%) with desired mass. The reaction mixture was concentrated under reduced pressure. The residue was purified by prep-HPLC (column Phenomenex luna C18 150*25 mm*10 um; mobile phase: [water (0.225% FA)-ACN]; B %: 4%-34%, 9 min) followed by lyophilization to give crude. The crude was purified by prep-HPLC (Phenomenex Gemini-NX C18 75*30 mm*3 um; mobile phase: [water (0.05% ammonia hydroxide v/v)-ACN]; B %: 30%-60%, 7 min) followed by lyophilization to give N-cyclobutyl-2-(2-((2,2,2-trifluoroethyl)amino)pyrimidin-4-yl)-1H-pyrrolo[3,2-c]pyridin-6-amine (11.79 mg, 0.032 mmol, 19.7% yield) as a yellow solid. ¹H NMR (400 MHz, MeOD) δ 8.38 (d, J=0.9 Hz, 1H), 8.27 (d, J=5.4 Hz, 1H), 7.22 (d, J=0.8 Hz, 1H), 7.17 (d, J=5.3 Hz, 1H), 6.39 (s, 1H), 4.43-4.28 (m, 2H), 4.12-4.08 (m, 1H), 2.50-2.48 (m, 2H), 2.98-1.86 (m, 4H); MS (ESI): m/z 363.3 [M+1]⁺.

### Example 33: (S)—N-(Tetrahydrofuran-3-yl)-2-(2-((2,2,2-trifluoroethyl)amino)pyrimidin-4-yl)-1H-pyrrolo[3,2-c]pyridin-6-amine

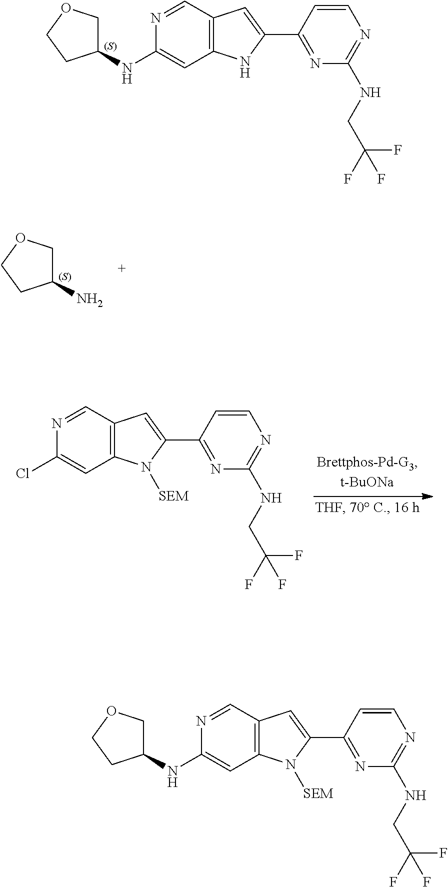

(S)—N-(Tetrahydrofuran-3-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine. To a solution of 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine (100 mg, 0.2200 mmol) in THF (2 mL) was added sodium tert-butoxide (0.22 mL, 0.44 mmol, 2 M in THF), methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (14 mg, 0.020 mmol) and rac-(3S)-tetrahydrofuran-3-amine (57 mg, 0.66 mmol), the reaction mixture was stirred at 70° C. for 16 h under nitrogen. LCMS showed 4-(6-chloro-1-((2-(trimethylsilyl)ethoxy)methyl)-1H-pyrrolo[3,2-c]pyridin-2-yl)-N-(2,2,2-trifluoroethyl)pyrimidin-2-amine was consumed completely and a peak (61%) with desired mass. The mixture was cooled and filtered with a pad of celite, the filtrate was concentrated under vacuum to give a residue, which was purified by silica gel chromatography (200-300 mesh silica gel, Petroleum ether/Ethyl acetate=100/1, 0/1, Petroleum ether/Ethyl acetate=0/1, Rf=0.4) to give (S)—N-(tetrahydrofuran-3-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine (95 mg, 0.1842 mmol, 84.38% yield) as a yellow solid. MS (ESI): m/z 509.1 [M+1]⁺.

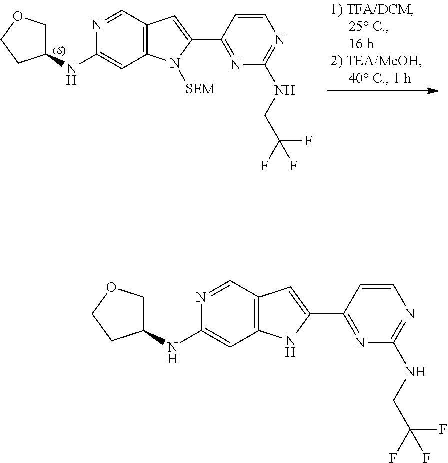

(S)—N-(Tetrahydrofuran-3-yl)-2-(2-((2,2,2-trifluoroethyl)amino)pyrimidin-4-yl)-1H-pyrrolo[3,2-c]pyridin-6-amine. To a solution of (S)—N-(tetrahydrofuran-3-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine (95 mg, 0.19 mmol) in dichloromethane (2 mL) was added trifluoroacetic acid (2 mL, 26.14 mmol), the reaction mixture was stirred at 25° C. for 16 h. LCMS showed (S)—N-(tetrahydrofuran-3-yl)-2-(2-((2,2,2-trifluoroethyl)amino)pyrimidin-4-yl)-1-((2-(trimethylsilyl)ethoxy)methyl)-1H-pyrrolo[3,2-c]pyridin-6-amine was consumed completely and a peak (89%) with desired mass. The reaction mixture was concentrated under reduced pressure. To the residue in methanol (2 mL) was added triethylamine (2 mL, 14.35 mmol), the reaction mixture was stirred at 40° C. for 1 h. LCMS showed a peak (97%) with desired mass. The residue was concentrated under reduced pressure. The residue was purified by prep-HPLC (column Phenomenex luna C18 150*25 mm*10 um; mobile phase: [water (0.225% FA)-ACN]; B %: 12%-32%, 10 min) followed by lyophilization to give (S)—N-(tetrahydrofuran-3-yl)-2-(2-((2,2,25trifluoroethyl)amino)pyrimidin-4-yl)-1H-pyrrolo[3,2-c]pyridin-6-amine (58.01 mg, 0.15 mmol, 79.5% yield) as a yellow solid. ¹H NMR (400 MHz, DMSO-d₆) δ 11.67 (s, 1H), 8.53 (s, 1H), 8.39 (d, J=5.0 Hz, 1H), 7.77-7.69 (m, 1H), 7.34 (s, 1H), 7.26 (d, J=5.1 Hz, 1H), 7.10-6.92 (m, 1H), 6.58 (s, 1H), 4.39-4.30 (m, 3H), 3.93-3.87 (m, 2H), 3.78-3.77 (m, 1H), 3.64-3.63 (m, 1H), 2.27-2.25 (m, 1H), 1.90-1.87 (m, 1H). MS (ESI): m/z 379.2 [M+1]⁺.

### Example 34: 2-(2-Ethoxypyrimidin-4-yl)-N-(tetrahydro-2H-pyran-4-yl)-1H-pyrrolo[3,2-c]pyridin-6-amine

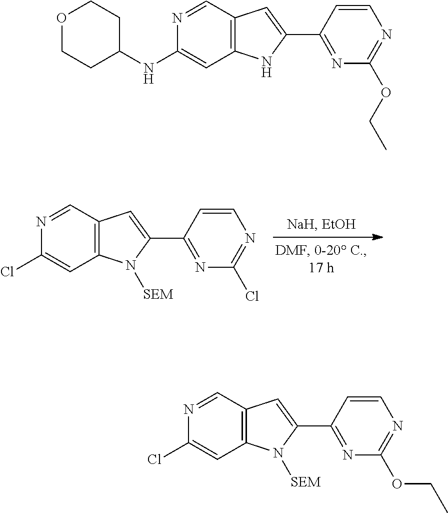 [SMILES: CCOc1nccc(-c2cc3cnc(NC4CCOCC4)cc3[nH]2)n1 ; C[Si](C)(C)CCOCn1c(-c2ccnc(Cl)n2)cc2cnc(Cl)cc21 ; CCOc1nccc(-c2cc3cnc(Cl)cc3n2COCC[Si](C)(C)C)n1]

6-Chloro-2-(2-ethoxypyrimidin-4-yl)-1-((2-(trimethylsilyl)ethoxy)methyl)-1H-pyrrolo[3,2-c]pyridine. To a solution of ethanol (52.4 mg, 1.14 mmol) in DMF (10 mL) was added sodium hydride (54.63 mg, 2.28 mmol) and stirred at 25° C. for 1 h. Then 6-chloro-2-(2-chloropyrimidin-4-yl)-1-((2-(trimethylsilyl)ethoxy)methyl)-1H-pyrrolo[3,2-c]pyridine (300 mg, 0.76 mmol) was added to the mixture. The mixture was stirred at 25° C. for 16 h. LCMS indicated that starting material was consumed and one major peak with desired mass. The residue was poured into water (5 mL) and extracted with ethyl acetate (20 mL×2). The combined organic phase was washed with brine (5 mL×2), dried over anhydrous sodium sulfate, filtered and concentrated under vacuum. The residue obtained was purified by silica gel chromatography (200-300 mesh silica gel, petroleum ether/ethyl acetate=10:1/3:1) to give 6-chloro-2-(2-ethoxypyrimidin-4-yl)-1-((2-(trimethylsilyl)ethoxy)methyl)-1H-pyrrolo[3,2-c]pyridine (200 mg, 0.47 mmol, 62.1% yield) as a yellow oil. ¹H NMR (400 MHz, DMSO-d₆) δ=8.79 (s, 1H), 8.69 (d, J=5.3 Hz, 1H), 7.95 (s, 10H), 7.93 (s, 1H), 7.70 (d, J=5.3 Hz, 1H), 7.59 (s, 1H), 6.21-6.16 (m, 2H), 4.42 (q, J=7.0 Hz, 2H), 1.38 (t, J=7.0 Hz, 4H), 0.69 (t, J=7.8 Hz, 2H), -0.24 (s, 9H); MS (ESI): m/z 405.0 [M+1]⁺.

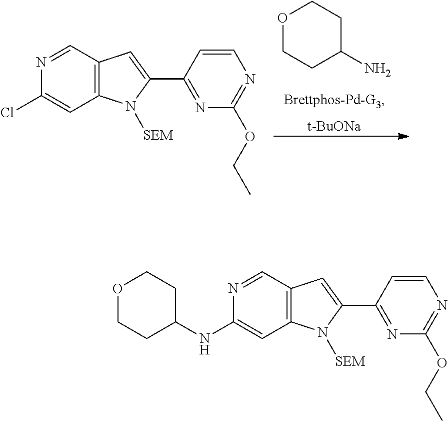

N-(2-(2-(Trifluoromethyl)pyridin-4-yl)-1-((2-(trimethylsilyl)ethoxy)methyl)-1H-pyrrolo[3,2-c]pyridin-6-yl)cyclopropanecarboxamide. To a solution of 6-chloro-2-(2-ethoxypyrimidin-4-yl)-1-((2-(trimethylsilyl)ethoxy)methyl)-1H-pyrrolo[3,2-c]pyridine (200 mg, 0.4900 mmol) and tetrahydropyran-4-amine (149 mg, 1.48 mmol) in anhydrous THE (2 mL) were added methanesulfonato(2-dicyclohexylphosphino-3,6-dimethoxy-2, 4,6-tri-i-propyl-1,1-biphenyl)(2-amino-1,1-biphenyl-2-yl)palladium(II) (45 mg, 0.050 mmol) and sodium tert-butoxide (0.49 mL, 0.99 mmol) (2 M in THF) under nitrogen, the mixture was stirred at 70° C. for 16 h. LCMS showed 6-chloro-2-(2-ethoxypyrimidin-4-yl)-1-((2-(trimethylsilyl)ethoxy)methyl)-1H-pyrrolo[3,2-c]pyridine was consumed completely and a peak with desired mass. The mixture was concentrated under vacuum. The residue obtained was purified by flash silica gel chromatography (Biotage, 12 g SepaFlash® Silica Flash Column, Eluent of 30˜70% Ethyl acetate/Petroleum ether gradient @ 20 mL/min) to give N-(2-(2-(trifluoromethyl)pyridin-4-yl)-1-((2-(trimethylsilyl)ethoxy)methyl)-1H-pyrrolo[3,2-c]pyridin-6-yl)cyclopropanecarboxamide (200 mg, 0.42 mmol, 86.1% yield) as a brown oil. ¹H NMR (400 MHz, CDCl₃) δ=8.54-8.45 (m, 2H), 7.30 (d, J=5.3 Hz, 1H), 7.09 (d, J=0.6 Hz, 1H), 6.38 (s, 1H), 6.08 (s, 2H), 4.51-4.45 (m, 2H), 4.07-3.93 (m, 3H), 3.86-3.75 (m, 1H), 3.63-3.56 (m, 2H), 3.48 (dd, J=7.6, 8.5 Hz, 2H), 2.15-2.07 (m, 2H), 1.64-1.59 (m, 2H), 1.48 (t, J=7.1 Hz, 3H), 0.85-0.80 (m, 2H), -0.12 (s, 9H); MS (ESI): m/z 470.3 [M+1]⁺.

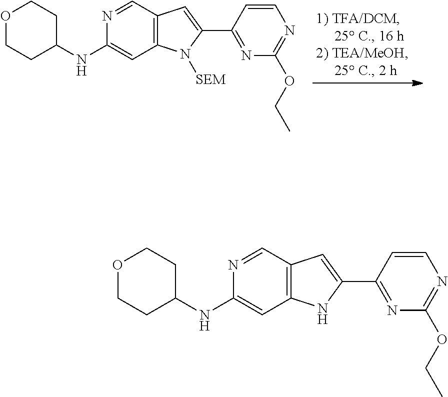 [SMILES: CCOc1nccc(-c2cc3cnc(NC4CCOCC4)cc3n2COCC[Si](C)(C)C)n1 ; CCOc1nccc(-c2cc3cnc(NC4CCOCC4)cc3[nH]2)n1]

2-(2-Ethoxypyrimidin-4-yl)-N-(tetrahydro-2H-pyran-4-yl)-1H-pyrrolo[3,2-c]pyridin-6-amine. To a solution of N-(2-(2-(trifluoromethyl)pyridin-4-yl)-1-((2-(trimethylsilyl)ethoxy)methyl)-1H-pyrrolo[3,2-c]pyridin-6-yl)cyclopropanecarboxamide (197.1 mg, 0.42 mmol) in dichloromethane (10 mL) was added trifluoroacetic acid (10 mL, 130.59 mmol) and stirred at 25° C. for 16 h. LCMS showed N-(2-(2-(trifluoromethyl)pyridin-4-yl)-1-((2-(trimethylsilyl)ethoxy)methyl)-1H-pyrrolo[3,2-c]pyridin-6-yl)cyclopropanecarboxamide was consumed completely and a major peak with desired mass. The mixture was concentrated under vacuum. To the residue obtained in methanol (10 mL), was added triethylamine (10.0 mL, 71.75 mmol). The solution was stirred at 25° C. for 1 h. LCMS showed N-(2-(2-(trifluoromethyl)pyridin-4-yl)-1-((2-(trimethylsilyl)ethoxy)methyl)-1H-pyrrolo[3,2-c]pyridin-6-yl)cyclopropanecarboxamide was consumed completely and a major peak with desired mass. The mixture was concentrated under vacuum to give a residue, which was purified by prep-HPLC (column: Shim-pack C18 150*25*10 um; mobile phase: water (0.225% FA)-ACN]; B %: 11%-44%, 11 min), then dried by lyophilization to give 2-(2-ethoxypyrimidin-4-yl)-N-(tetrahydro-2H-pyran-4-yl)-1H-pyrrolo[3,2-c]pyridin-6-amine (68.1 mg, 0.20 mmol, 47.7% yield) as a yellow solid. ¹H NMR (400 MHz, DMSO-d₆) δ 11.79-11.74 (m, 1H), 8.59 (d, J=5.1 Hz, 1H), 8.52 (s, 1H), 7.63 (d, J=5.3 Hz, 1H), 7.42 (d, J=1.1 Hz, 1H), 6.90-6.77 (m, 1H), 6.62-6.54 (m, 1H), 4.45 (q, J=7.1 Hz, 2H), 3.94-3.86 (m, 2H), 3.85-3.74 (m, 1H), 3.49-3.41 (m, 2H), 1.98-1.84 (m, 2H), 1.54-1.41 (m, 2H), 1.37 (t, J=7.1 Hz, 3H); MS (ESI): m/z 340.2 [M+1]⁺.

### Example 35: 1-Methyl-2-(2-methylpyrimidin-4-yl)-N-tetrahydropyran-4-yl-pyrrolo[3,2-c]pyridin-6-amine

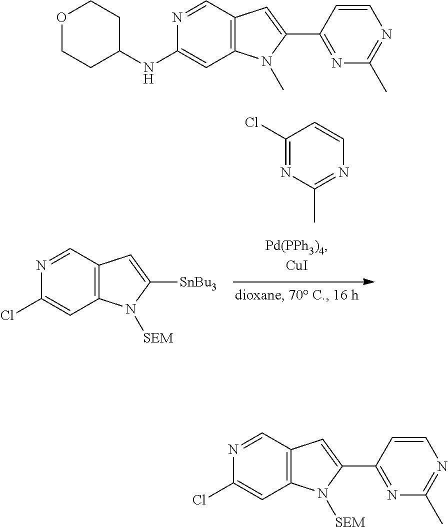

2-[[6-Chloro-2-(2-methylpyrimidin-4-yl)pyrrolo[3,2-c]pyridin-1-yl]methoxy]ethyl-trimethyl-silane. To a mixture of 2-[(6-chloro-2-tributylstannyl-pyrrolo[3,2-c]pyridin-1-yl)methoxy]ethyl -trimethyl-silane (1.5 g, 2.62 mmol) in 1,4-Dioxane (15 mL) was added 4-chloro-2-methyl-pyrimidine (0.67 g, 5.25 mmol), cuprous iodide (49.95 mg, 0.26 mmol) and tetrakis[triphenylphosphine]palladium(0) (303.09 mg, 0.26 mmol). The mixture was stirred at 70° C. for 16 h under nitrogen. LCMS showed a peak (42%) with desired mass. The reaction mixture was filtered and the filtrate was concentrated. The residue was purified by flash silica gel chromatography (20 g SepaFlash® Silica Flash Column, Eluent of 30˜40% Ethyl acetate/Petroleum ether gradient) to give 2-[[6-chloro-2-(2-methylpyrimidin-4-yl)pyrrolo[3,2-c]pyridin-1-yl]methoxy]ethyl-trimethyl-silane (700 mg, 1.83 mmol, 69.97% yield) as a yellow solid. MS (ESI): m/z 375.1 M+1]⁺.

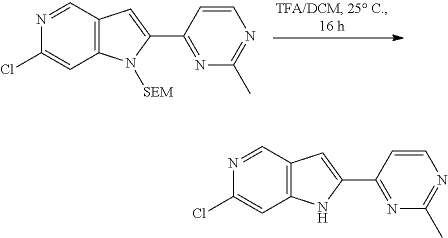 [SMILES: Cc1nccc(-c2cc3cnc(Cl)cc3n2COCC[Si](C)(C)C)n1 ; Cc1nccc(-c2cc3cnc(Cl)cc3[nH]2)n1]

6-Chloro-2-(2-methylpyrimidin-4-yl)-1H-pyrrolo[3,2-c]pyridine. To a mixture of 2-[[6-chloro-2-(2-methylpyrimidin-4-yl)pyrrolo[3,2-c]pyridin-1-yl]methoxy]ethyl-trimethyl-silane (700 mg, 1.87 mmol) in dichloromethane (10 mL) was added trifluoroacetic acid (10. mL, 129.8 mmol), The mixture was stirred at 25° C. for 16 h. LCMS showed a peak (75%) with desired mass. The reaction mixture was concentrated under reduced pressure. The residue obtained was dilute with methanol (10 mL) and adjusted to pH=9 with triethylamine. The reaction mixture was concentrated under reduced pressure to give a crude product. The crude product was triturated with methanol (5 mL) for 3 h to give 6-chloro-2-(2-methylpyrimidin-4-yl)-1H-pyrrolo[3,2-c]pyridine (500 mg, crude) as a yellow solid. MS (ESI): m/z 375.1 M+1]⁺.

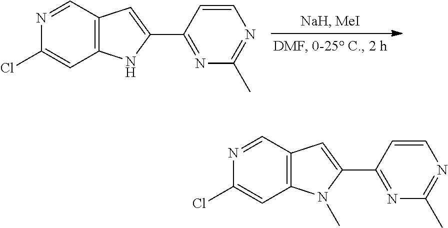

6-Chloro-1-methyl-2-(2-methylpyrimidin-4-yl)pyrrolo[3,2-c]pyridine. To a mixture of 6-chloro-2-(2-methylpyrimidin-4-yl)-1H-pyrrolo[3,2-c]pyridine (500 mg, 2.04 mmol) in DMF (6 mL) was added sodium hydride (73 mg, 3.07 mmol) at 0° C. The mixture was stirred at 25° C. for 0.5 h. Iodomethane (0.25 mL, 4.09 mmol) was added at 0° C. The mixture was stirred at 25° C. for 1.5 h. LCMS showed a peak (92%) with desired mass. The mixture was poured into cold saturated ammonium chloride (100 mL). The aqueous phase was extracted with ethyl acetate (100 mL×3). The combined organic phase was washed with brine (50 mL×4), dried over anhydrous sodium sulfate, filtered and concentrated under vacuum. The residue was purified by flash silica gel chromatography (20 g SepaFlash® Silica Flash Column, Eluent of 60˜80% Ethyl acetate/Petroleum ether gradient) to give 6-chloro-1-methyl-2-(2-methylpyrimidin-4-yl)pyrrolo[3,2-c]pyridine (400 mg, 1.52 mmol, 74.3% yield) as a yellow solid. MS (ESI): m/z 259.1 M+1]⁺.

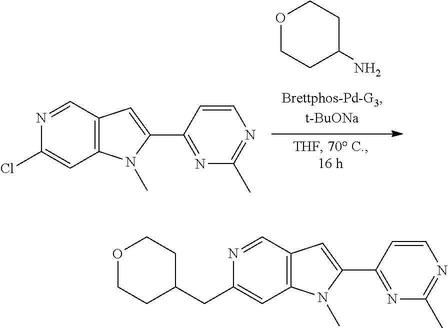

1-Methyl-2-(2-methylpyrimidin-4-yl)-N-tetrahydropyran-4-yl-pyrrolo[3,2-c]pyridin-6-amine. To a mixture of 6-chloro-1-methyl-2-(2-methylpyrimidin-4-yl)pyrrolo[3,2-c]pyridine (400 mg, 1.55 mmol) in tetrahydrofuran (7 mL) was added tetrahydropyran-4-amine (469 mg, 4.64 mmol), Methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (280 mg, 0.31 mmol) and Sodium tert-butoxide (1.55 mL, 3.09 mmol). The mixture was stirred at 70° C. for 16 h under nitrogen. LCMS showed a peak (48%) with desired mass. The reaction mixture was filtered and the filtrate was concentrated. The residue was purified by prep-HPLC (column: Phenomenex Luna C18 150*40 mm*15 um; mobile phase: [water (0.225% FA)-ACN]; B %: 1%-25%, 10 min) followed by lyophilization to give 1-methyl-2-(2-methylpyrimidin-4-yl)-N-tetrahydropyran-4-yl-pyrrolo[3,2-c]pyridin-6-amine (224.91 mg, 0.69 mmol, 44.8% yield, formic acid) as a yellow solid. MS (ESI): m/z 324.2 M+1]⁺; ¹H NMR (400 MHz, DMSO-d₆) δ=8.63 (d, J=5.4 Hz, 1H), 8.42 (s, 1H), 8.16 (s, 1H), 7.71 (d, J=5.4 Hz, 1H), 7.23 (s, 1H), 6.38 (s, 1H), 6.13 (d, J=7.6 Hz, 1H), 3.98 (s, 3H), 3.92-3.85 (m, 3H), 3.46-3.40 (m, 2H), 2.65 (s, 3H), 1.96-1.86 (m, 2H), 1.52-1.38 (m, 2H).

### Example 36: 1-Methyl-N-[rac-(3s)-tetrahydropyran-3-yl]-2-[2-(2,2,2-trifluoroethylamino)pyrimidin-4-yl]pyrrolo[3,2-c]pyridin-6-amine

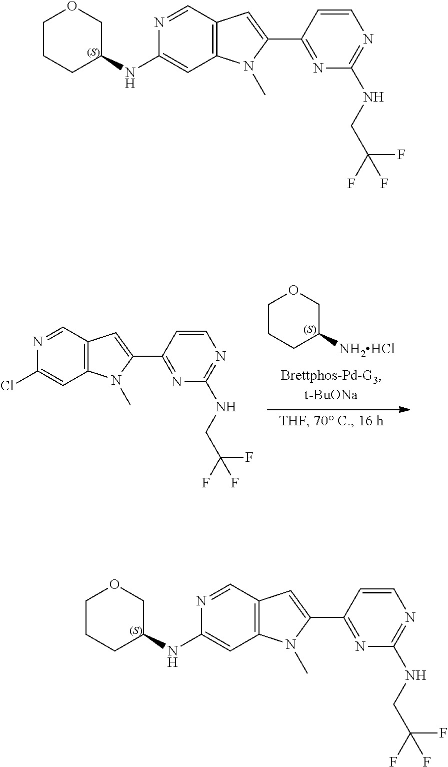

1-Methyl-N-[rac-(3S)-tetrahydropyran-3-yl]-2-[2-(2,2,2-trifluoroethylamino)pyrimidin-4-yl]pyrrolo[3,2-c]pyridin-6-amine. To 4-(6-chloro-1-methyl-pyrrolo[3,2-c]pyridin-2-yl)-N-(2,2,2-trifluoroethyl)pyrimidin-2-amine (300 mg, 0.88 mmol) in tetrahydrofuran (5 mL) was added rac-(3S)-tetrahydropyran-3-amine; hydrochloride (362 mg, 2.63 mmol), methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (159 mg, 0.18 mmol), and sodium tert-butoxide (0.88 mL, 1.76 mmol, 1M). The mixture was stirred at 70° C. for 16 h under nitrogen. LCMS showed a peak (40%) with desired mass. The reaction mixture was filtered and the filtrate was concentrated. The residue was purified by prep-HPLC (column: Phenomenex Luna C18 150*40 mm*15 um; mobile phase: [water (0.225% FA)-ACN]; B %: 10%-40%, 10 min) followed by lyophilization to give 1-methyl-N-[rac-(3S)-tetrahydropyran-3-yl]-2-[2-(2,2,2-trifluoroethylamino)pyrimidin-4-yl]pyrrolo[3,2-c]pyridin-6-amine (103.41 mg, 0.25 mmol, 28.5% yield, formic acid) as a yellow solid. ¹H NMR (400 MHz, DMSO-d₆) δ 8.39 (s, 1H), 8.33 (d, J=5.3 Hz, 1H), 8.17 (s, 0.6H), 7.81 (t, J=6.7 Hz, 1H), 7.18-7.13 (m, 2H), 6.41 (s, 1H), 6.03 (d, J=8.3 Hz, 1H), 4.23-4.12 (m, 2H), 3.96 (s, 3H), 3.91-3.72 (m, 1H), 3.75-3.55 (m, 1H), 3.77-3.72 (m, 1H), 3.36-3.32 (m, 1H), 3.14-3.10 (m, 1H), 2.03-1.94 (m, 1H), 1.75-1.48 (m, 3H); MS (ESI): m/z 407.1 [M+1]⁺.

### Example 37: N-(Tetrahydro-2H-pyran-4-yl)-2-(2-(2,2,2-trifluoroethoxy)pyrimidin-4-yl)-1H-pyrrolo[3,2-c]pyridin-6-amine

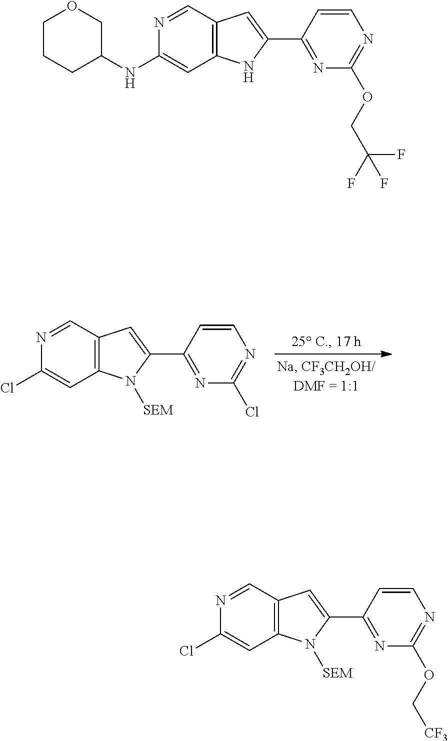 [SMILES: FC(F)(F)COc1nccc(-c2cc3cnc(NC4CCCOC4)cc3[nH]2)n1 ; C[Si](C)(C)CCOCn1c(-c2ccnc(Cl)n2)cc2cnc(Cl)cc21 ; C[Si](C)(C)CCOCn1c(-c2ccnc(OCC(F)(F)F)n2)cc2cnc(Cl)cc21]

6-Chloro-2-(2-(2,2,2-trifluoroethoxy)pyrimidin-4-yl)-1-((2-(trimethylsilyl)ethoxy)methyl)-1H-pyrrolo[3,2-c]pyridine. To a solution of 2, 2,2-trifluoroethanol (3 mL) in DMF (3 mL) was added sodium (48.56 mg, 2.02 mmol) and stirred at 25° C. for 1 h. Then 6-chloro-2-(2-chloropyrimidin-4-yl)-1-((2-(trimethylsilyl)ethoxy)methyl)-1H-pyrrolo[3,2-c]pyridine (400. mg, 1.01 mmol) was added to the mixture. The mixture was stirred at 25° C. for 16 h. LCMS indicated starting material was consumed and one major peak with desired mass. The residue was poured into water (5 mL). The aqueous phase was extracted with ethyl acetate (20 mL×2). The combined organic phase was washed with brine (5 mL×2), dried over anhydrous sodium sulfate, filtered and concentrated under vacuum. The residue was purified by silica gel chromatography (200-300 mesh silica gel, petroleum ether/ethyl acetate=10:1/3:1) to give 6-chloro-2-(2-(2,2,2-trifluoroethoxy)pyrimidin-4-yl)-1-((2-(trimethylsilyl)ethoxy)methyl)-1H-pyrrolo[3,2-c]pyridine (200 mg, 0.4716 mmol, 62.156% yield) as a yellow oil. ¹H NMR (400 MHz, DMSO-d₆) δ=8.80 (s, 1H), 8.77 (d, J=5.1 Hz, 1H), 7.95 (s, 1H), 7.85 (d, J=5.3 Hz, 1H), 7.66 (s, 1H), 6.12 (s, 2H), 5.17-5.08 (m, 2H), 3.44-3.39 (m, 2H), 0.71 (t, J=7.8 Hz, 2H), -0.22 (s, 9H), LCMS (ESI): m/z 459.1 [M+H]+.

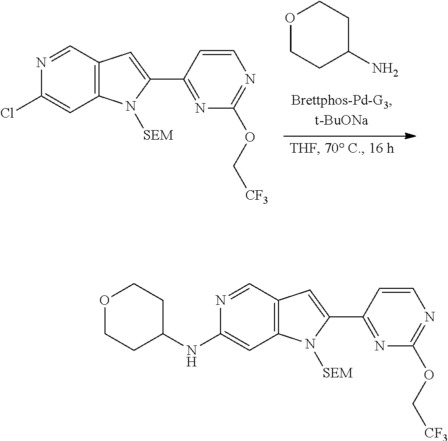 [SMILES: C[Si](C)(C)CCOCn1c(-c2ccnc(OCC(F)(F)F)n2)cc2cnc(Cl)cc21 ; NC1CCOCC1 ; C[Si](C)(C)CCOCn1c(-c2ccnc(OCC(F)(F)F)n2)cc2cnc(NC3CCOCC3)cc21]

N-(Tetrahydro-2H-pyran-4-yl)-2-(2-(2,2,2-trifluoroethoxy)pyrimidin-4-yl)-1-((2-(trimethylsilyl)ethoxy)methyl)-1H-pyrrolo[3,2-c]pyridin-6-amine. To a solution of tetrahydropyran-4-amine (132.24 mg, 1.31 mmol) and 6-chloro-2-(2-(2,2,2-trifluoroethoxy)pyrimidin-4-yl)-1-((2-(trimethylsilyl)ethoxy)methyl)-1H-pyrrolo[3,2-c]pyridine (300. mg, 0.6500 mmol) in THF (5 mL) was added sodium tert-butoxide (0.65 mL, 1.31 mmol), and methanesulfonato(2-dicyclohexylphosphino-3,6-dimethoxy-2, 4,6-tri-i-propyl-1,1-biphenyl)(2-amino-1,1-biphenyl-2-yl)palladium(II) (59.86 mg, 0.0700 mmol), the reaction mixture was stirred at 70° C. for 16 h under nitrogen. LCMS showed 6-chloro-2-(2-(2,2,2-trifluoroethoxy)pyrimidin-4-yl)-1-((2-(trimethylsilyl)ethoxy)methyl)-1H-pyrrolo[3,2-c]pyridine was consumed completely and a major peak with desired mass. The mixture was cooled and filtered with a pad of Celite, the filtrate was concentrated under vacuum to give a residue. The residue was purified by silica gel chromatography (200-300 mesh silica gel, petroleum ether/ethyl acetate=100/1, 2/1, Rf=0.5 petroleum ether/ethyl acetate=1/1) to give N-(tetrahydro-2H-pyran-4-yl)-2-(2-(2,2,2-trifluoroethoxy)pyrimidin-4-yl)-1-((2-(trimethylsilyl)ethoxy)methyl)-1H-pyrrolo[3,2-c]pyridin-6-amine (200 mg, 0.4192 mmol, 78.002% yield) as a yellow oil. LCMS (ESI): m/z 524.2 [M+1]⁺.

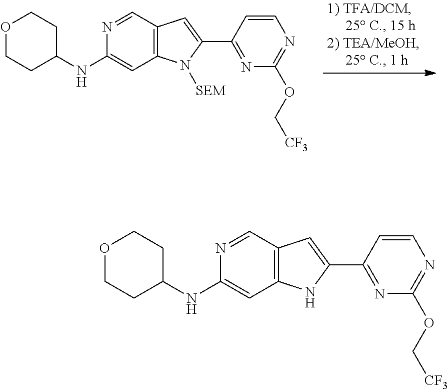 [SMILES: C[Si](C)(C)CCOCn1c(-c2ccnc(OCC(F)(F)F)n2)cc2cnc(NC3CCOCC3)cc21 ; FC(F)(F)COc1nccc(-c2cc3cnc(NC4CCOCC4)cc3[nH]2)n1]

2-(2-Ethoxypyrimidin-4-yl)-N-(tetrahydro-2H-pyran-4-yl)-1H-pyrrolo[3,2-c]pyridin-6-amine. To a solution of N-(tetrahydro-2H-pyran-4-yl)-2-(2-(2,2,2-trifluoroethoxy)pyrimidin-4-yl)-1-((2-(trimethylsilyl)ethoxy)methyl)-1H-pyrrolo[3,2-c]pyridin-6-amine (200. mg, 0.3800 mmol) in dichloromethane (10 mL) was added trifluoroacetic acid (10.0 mL, 130.59 mmol) and stirred at 25° C. for 16 h. LCMS showed N-(tetrahydro-2H-pyran-4-yl)-2-(2-(2,2,2-trifluoroethoxy)pyrimidin-4-yl)-1-((2-(trimethylsilyl)ethoxy)methyl)-1H-pyrrolo[3,2-c]pyridin-6-amine was consumed completely and a major peak with desired mass. The mixture was concentrated under vacuum The residue was dissolvent in methanol (10 mL), the triethylamine (10. mL, 71.75 mmol) was added to the solution, stirred at 25° C. for 1 h. LCMS showed N-(tetrahydro-2H-pyran-4-yl)-2-(2-(2,2,2-trifluoroethoxy)pyrimidin-4-yl)-1-((2-(trimethylsilyl)ethoxy)methyl)-1H-pyrrolo[3,2-c]pyridin-6-amine was consumed completely and a major peak with desired mass. The mixture was concentrated under vacuum to give a residue. The residue was purified by prep-HPLC (column: Phenomenex luna C18 150*25 mm*10 um; mobile phase: water(0.225% FA)-ACN];B %: 18%-48%, 11 min), then dried by lyophilization to give 2-(2-ethoxypyrimidin-4-yl)-N-(tetrahydro-2H-pyran-4-yl)-1H-pyrrolo[3,2-c]pyridin-6-amine (98.99 mg, 0.2479 mmol, 64.897% yield) as a yellow solid. Additional 2.27 mg for delivery. ¹H NMR (400 MHz, DMSO-d₆) δ=11.97 (br s, 1H), 8.69 (d, J=5.3 Hz, 1H), 8.58 (s, 1H), 7.83 (d, J=5.3 Hz, 1H), 7.55 (s, 1H), 7.17-6.98 (m, 1H), 6.64 (s, 1H), 5.20 (q, J=9.1 Hz, 2H), 3.95-3.87 (m, 2H), 3.85-3.75 (m, 1H), 3.46-3.41 (m, 2H), 1.93-1.90 (m, 2H), 1.54-1.42 (m, 2H). LCMS (ESI): m/z 394.2 [M+1]⁺.

### Example 38: 2-(2-(Methyl(2,2,2-trifluoroethyl)amino)pyridin-4-yl)-N-(tetrahydro-2H-pyran-4-yl)-1H-pyrrolo[3,2-c]pyridin-6-amine

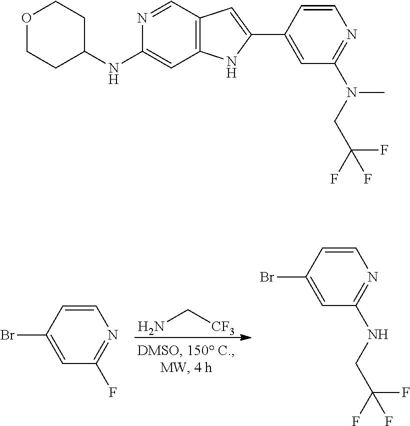 [SMILES: CN(CC(F)(F)F)c1cc(-c2cc3cnc(NC4CCOCC4)cc3[nH]2)ccn1 ; Fc1cc(Br)ccn1 ; NCC(F)(F)F ; FC(F)(F)CNc1cc(Br)ccn1]

4-Bromo-N-(2,2,2-trifluoroethyl)pyridin-2-amine. The reaction was paralleled for three batches and 2 g for each: To a mixture of 4-bromo-2-fluoropyridine (6. g, 34.09 mmol) in DMSO (90 mL) was added 2,2,2-trifluoroethanamine (39.13 mL, 681.86 mmol). The mixture was stirred at 150° C. for 4 h under microwave. LCMS showed 4-bromo-2-fluoropyridine was consumed completely and a peak (60%) with desired mass. The residue was poured into water (20 mL). The aqueous phase was extracted with ethyl acetate (30 mL×3). The combined organic phase was washed with brine (20 mL×2), dried over anhydrous sodium sulfate, filtered and concentrated under vacuum. The residue was purified by flash silica gel chromatography (Biotage, 40 g SepaFlash® Silica Flash Column, eluent of 0˜5% ethyl acetate/petroleum ether gradient @ 60 mL/min) to give 4-bromo-N-(2,2,2-trifluoroethyl)pyridin-2-amine (3 g, 11.763 mmol, 34.502% yield) as a white solid. LCMS (ESI): m/z=255.0 [M+1]⁺.

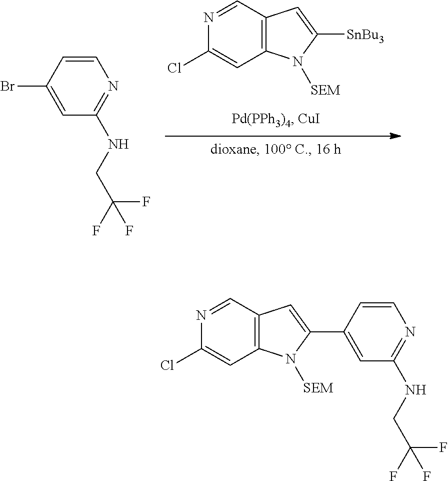

4-(6-Chloro-1-((2-(trimethylsilyl)ethoxy)methyl)-1H-pyrrolo[3,2-c]pyridin-2-yl)-N-(2,2,2-trifluoroethyl)pyridin-2-amine. To a mixture of 4-bromo-N-(2,2,2-trifluoroethyl)pyridin-2-amine (1. g, 3.92 mmol) and 6-chloro-2-(tributylstannyl)-1-((2-(trimethylsilyl)ethoxy)methyl)-1H-pyrrolo[3,2-c]pyridine (2.24 g, 3.92 mmol) in 1,4-dioxane (10 mL) was added copper(l) iodide (1.49 g, 7.84 mmol) and tetrakis(triphenylphosphine) palladium(0) (453.11 mg, 0.3900 mmol). The mixture was stirred at 100° C. for 16 h under nitrogen atmosphere. LCMS showed 6-chloro-2-(tributylstannyl)-1-((2-(trimethylsilyl)ethoxy)methyl)-1H-pyrrolo[3,2-c]pyridine was consumed completely and a peak (45%) with desired mass. The mixture was concentrated under reduced pressure to give a residue. The residue was purified by prep-HPLC (column: Phenomenex luna Cis 150*40 mm*15 um; mobile phase: [water(0.225% FA) -ACN];B %: 57%-87%, 10 min), then dried by lyophilization to give 4-(6-chloro-1-((2-(trimethylsilyl)ethoxy)methyl)-1H-pyrrolo[3,2-c]pyridin-2-yl)-N-(2,2,2-trifluoroethyl)pyridin-2-amine (500 mg, 1.0942 mmol, 27.906% yield) as a yellow solid. ¹H NMR (400 MHz, DMSO-d₆) δ 8.71 (s, 1H), 8.20-8.11 (m, 1H), 7.91-7.84 (m, 1H), 7.34 (t, J=6.6 Hz, 1H), 6.94-6.90 (m, 2H), 6.90-6.85 (m, 1H), 5.60 (s, 2H), 4.33-4.08 (m, 2H), 3.45-3.38 (m, 2H), 0.83-0.69 (m, 2H), -0.14 (s, 9H).

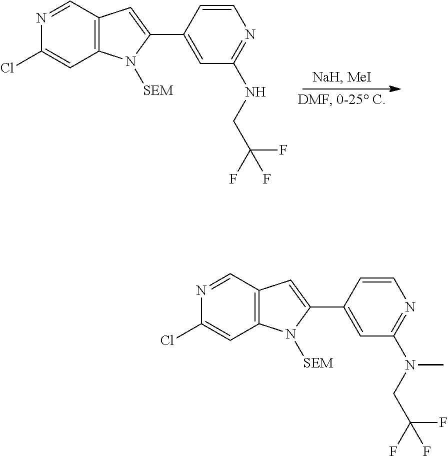 [SMILES: C[Si](C)(C)CCOCn1c(-c2ccnc(NCC(F)(F)F)c2)cc2cnc(Cl)cc21 ; CN(CC(F)(F)F)c1cc(-c2cc3cnc(Cl)cc3n2COCC[Si](C)(C)C)ccn1]

4-(6-Chloro-1-((2-(trimethylsilyl)ethoxy)methyl)-1H-pyrrolo[3,2-c]pyridin-2-yl)-N-methyl-N-(2,2,2-trifluoroethyl)pyridin-2-amine. To a mixture of 4-(6-chloro-1-((2-(trimethylsilyl)ethoxy)methyl)-1H-pyrrolo[3,2-c]pyridin-2-yl)-N-(2,2,2-trifluoroethyl)pyridin-2-amine (500. mg, 1.09 mmol) in DMF (20 mL) was added sodium hydride (87.54 mg, 2.19 mmol) at 0° C., the mixture was stirred at 0° C. for 0.5 h. Then iodomethane (232.96 mg, 1.64 mmol) was added to the mixture at 0° C. The mixture was stirred at 25° C. for 1 h. TLC (Petroleum ether:ethyl acetate=2:1) indicated starting material was consumed and one major new spot with lower polarity. The residue was poured into water (30 mL). The aqueous phase was extracted with ethyl acetate (30 mL×3). The combined organic phase was washed with brine (20 mL×2), dried over anhydrous sodium sulfate, filtered and concentrated under vacuum. The residue was purified by silica gel chromatography (100-200 mesh silica gel, Petroleum ether/ethyl acetate=0/1, 3/1) to give 4-(6-chloro-1-((2-(trimethylsilyl)ethoxy)methyl)-1H-pyrrolo[3,2-c]pyridin-2-yl)-N-methyl-N-(2,2,2-trifluoroethyl)pyridin-2-amine (500 mg, 1.0478 mmol, 95.76% yield) was obtained as a yellow solid. ¹H NMR (400 MHz, DMSO-d₆) δ 8.71 (s, 1H), 8.25 (d, J=5.1 Hz, 1H), 7.91 (s, 1H), 7.08 (s, 1H), 7.05-7.01 (m, 2H), 5.62 (s, 2H), 4.55 (q, J=9.6 Hz, 2H), 3.51-3.44 (m, 2H), 3.15 (s, 3H), 0.81-0.73 (m, 2H), -0.13 (s, 9H). LCMS (ESI): m/z=471.1 [M+1]⁺.

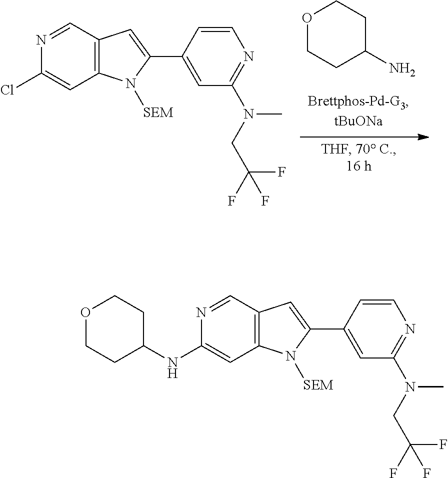 [SMILES: CN(CC(F)(F)F)c1cc(-c2cc3cnc(Cl)cc3n2COCC[Si](C)(C)C)ccn1 ; NC1CCOCC1 ; CN(CC(F)(F)F)c1cc(-c2cc3cnc(NC4CCOCC4)cc3n2COCC[Si](C)(C)C)ccn1]

2-(2-(Methyl(2,2,2-trifluoroethyl)amino)pyridin-4-yl)-N-(tetrahydro-2H-pyran-4-yl)-1-((2-(trimethylsilyl)ethoxy)methyl)-1H-pyrrolo[3,2-c]pyridin-6-amine. To a mixture of 4-(6-chloro-1-((2-(trimethylsilyl)ethoxy)methyl)-1H-pyrrolo[3,2-c]pyridin-2-yl)-N-methyl-N-(2,2,2-trifluoroethyl)pyridin-2-amine (500. mg, 1.06 mmol) and tetrahydropyran-4-amine (322.14 mg, 3.18 mmol) in THE (1 mL) was added methanesulfonato(2-dicyclohexylphosphino-3,6-dimethoxy-2,4,6-tri-i-propyl-1,1-biphenyl)(2-amino-1,1-biphenyl-2-yl)palladium(II) (96.23 mg, 0.1100 mmol) and sodium tert-butoxide (1.06 mL, 2.12 mmol). (2 M in THF), The mixture was stirred at 70° C. for 16 h. LCMS showed 4-(6-chloro-1-((2-(trimethylsilyl)ethoxy)methyl)-1H-pyrrolo[3,2-c]pyridin-2-yl)-N-methyl-N-(2,2,2-trifluoroethyl)pyridin-2-amine was consumed completely and a major peak with desired mass. The residue was purified by silica gel chromatography (100-200 mesh silica gel, Petroleum ether/Ethyl acetate=0/1, 1/0) to give 2-(2-(methyl(2,2,2-trifluoroethyl)amino)pyridin-4-yl)-N-(tetrahydro-2H-pyran-4-yl)-1-((2-(trimethylsilyl)ethoxy)methyl)-1H-pyrrolo[3,2-c]pyridin-6-amine (500 mg, 0.8858 mmol, 83.44% yield) as a yellow solid. ¹H NMR (400 MHz, DMSO-d₆) δ 8.45 (s, 1H), 8.27 (d, J=5.1 Hz, 1H), 7.11 (s, 1H), 7.07 (d, J=5.1 Hz, 1H), 6.86 (s, 1H), 6.64 (s, 1H), 6.16 (d, J=8.1 Hz, 1H), 5.50 (s, 2H), 4.62 (q, J=9.5 Hz, 2H), 4.01-3.93 (m, 3H), 3.63-3.56 (m, 2H), 3.55-3.46 (m, 2H), 3.23 (s, 3H), 2.03-1.93 (m, 2H), 1.57-1.47 (m, 2H), 0.95-0.89 (m, 2H), 0.00 (s, 9H), LCMS (ESI): m/z=536.2 [M+1]⁺.

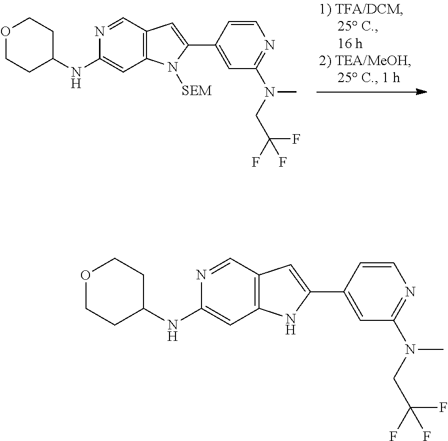 [SMILES: CN(CC(F)(F)F)c1cc(-c2cc3cnc(NC4CCOCC4)cc3n2COCC[Si](C)(C)C)ccn1 ; CN(CC(F)(F)F)c1cc(-c2cc3cnc(NC4CCOCC4)cc3[nH]2)ccn1]

2-(2-(Methyl(2,2,2-trifluoroethyl)amino)pyridin-4-yl)-N-(tetrahydro-2H-pyran-4-yl)-1H-pyrrolo[3,2-c]pyridin-6-amine. To a solution of 2-(2-(methyl(2,2,2-trifluoroethyl)amino)pyridin-4-yl)-N-(tetrahydro-2H-pyran-4-yl)-1-((2-(trimethylsilyl)ethoxy)methyl)-1H-pyrrolo[3,2-c]pyridin-6-amine (500. mg, 0.9300 mmol) in dichloromethane (10 mL) was added trifluoroacetic acid (22.24 mL, 290.45 mmol) and stirred at 25° C. for 16 h. LCMS showed 2-(2-(methyl(2,2,2-trifluoroethyl)amino)pyridin-4-yl)-N-(tetrahydro-2H-pyran-4-yl)-1-((2-(trimethylsilyl)ethoxy)methyl)-1H-pyrrolo[3,2-c]pyridin-6-amine was consumed completely and a major peak with desired mass. The mixture was concentrated under vacuum. The residue was dissolved in methanol (10 mL), the triethylamine (22.24 mL, 159.57 mmol) was added to the solution and stirred at 25° C. for 1 h. LCMS showed 2-[2-[methyl(2,2,2-trifluoroethyl)amino]-4-pyridyl]-N-tetrahydropyran-4-yl-1-(2-trimethylsilylethoxymethyl)pyrrolo[3,2-c]pyridin-6-amine was consumed completely and a major peak with desired mass. The mixture was concentrated under vacuum to give a residue. The residue was purified by prep-HPLC (column: Phenomenex luna C18 150*40 mm*15 um; mobile phase: water(0.225% FA)-ACN];B %: 6%-36%, 11 min), then dried by lyophilization, HNMR showed impurities, the crude product was purified by 2^{nd} prep-HPLC (column: Phenomenex Synergi C18 150*25 mm*10 um; mobile phase: water(0.225% FA)-ACN];B %: 15%-45%, 11 min), then dried by lyophilization to give 2-(2-(methyl(2,2,2-trifluoroethyl)amino)pyridin-4-yl)-N-(tetrahydro-2H-pyran-4-yl)-1H-pyrrolo[3,2-c]pyridin-6-amine (256.64 mg, 0.6324 mmol, 67.752% yield) (formic acid) as a yellow solid. ¹H NMR (400 MHz, DMSO-d₆) δ 13.55-13.13 (m, 1H), 12.43-12.33 (m, 1H), 8.55 (s, 1H), 8.23 (d, J=5.3 Hz, 1H), 7.67 (s, 1H), 7.31 (d, J=1.3 Hz, 1H), 7.23-7.18 (m, 2H), 6.80 (s, 1H), 4.59-4.53 (m, 2H), 3.94-3.88 (m, 2H), 3.83-3.75 (m, 1H), 3.47-3.42 (m, 2H), 3.19 (s, 3H), 1.97-1.93 (m, 2H), 1.58-1.42 (m, 2H). LCMS (ESI): m/z=406.2 [M+1]⁺.

### Example 39: 1-Methyl-N-(tetrahydro-2H-pyran-4-yl)-2-(2-((2,2,2-trifluoroethyl)amino)pyridin-4-yl)-1H-pyrrolo[3,2-c]pyridin-6-amine

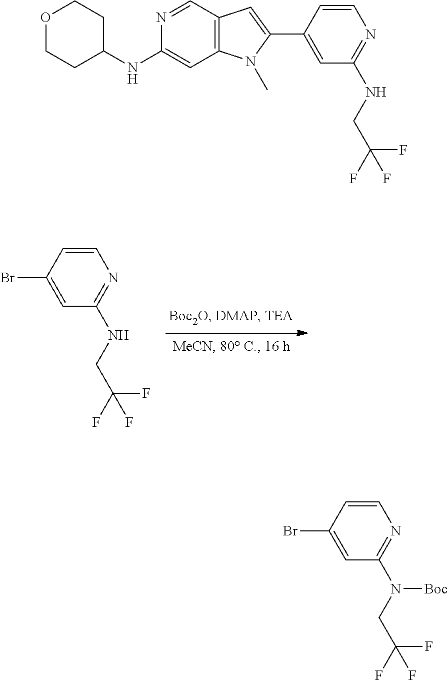 [SMILES: Cn1c(-c2ccnc(NCC(F)(F)F)c2)cc2cnc(NC3CCOCC3)cc21 ; FC(F)(F)CNc1cc(Br)ccn1 ; CC(C)(C)OC(=O)N(CC(F)(F)F)c1cc(Br)ccn1]

tert-Butyl (4-bromopyridin-2-yl)(2,2,2-trifluoroethyl)carbamate. To a solution of 4-bromo-N-(2,2,2-trifluoroethyl)pyridin-2-amine (2.5 g, 9.8 mmol), triethylamine (1983.81 mg, 19.6 mmol) and 4-dimethylaminopyridine (1197.56 mg, 9.8 mmol) in acetonitrile (30 mL) was added di-tert-butyl dicarbonate (3209.06 mg, 14.7 mmol), the mixture was stirred at 80° C. for 16 h. LCMS showed 4-bromo-N-(2,2,2-trifluoroethyl)pyridin-2-amine was consumed completely and a peak (96%) with desired mass. The mixture was concentrated under vacuum. The residue was purified by flash silica gel chromatography (Biotage, 40 g SepaFlash® Silica Flash Column, Eluent of 0˜10% Ethyl acetate/Petroleum ether gradient @ 35 mL/min) to give tert-butyl N-(4-bromo-2-pyridyl)-N-(2,2,2-trifluoroethyl)carbamate (2.1 g, 5.913 mmol, 60.322% yield) as a colorless oil. ¹H NMR (400 MHz, CDCl₃) δ 8.18 (d, J=5.3 Hz, 1H), 7.97 (s, 1H), 7.24 (dd, J₁=5.3, J₂=1.5 Hz, 1H), 4.81 (q, J=8.7 Hz, 2H), 1.54 (s, 9H); LCMS (ESI): m/z 299.9 [M-55]⁺.

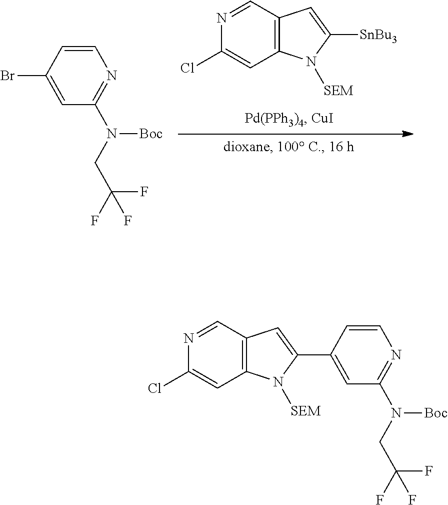

tert-Butyl (4-(6-chloro-1-((2-(trimethylsilyl)ethoxy)methyl)-1H-pyrrolo[3,2-c]pyridin-2-yl)pyridin-2-yl)(2,2,2-trifluoroethyl)carbamate. To a solution of 2-[(6-chloro-2-tributylstannyl-pyrrolo[3,2-c]pyridin-1-yl)methoxy]ethyl-trimethyl-silane (2. g, 3.5 mmol) and tert-butyl N-(4-bromo-2-pyridyl)-N-(2,2,2-trifluoroethyl)carbamate (1.24 g, 3.5 mmol) in 1,4-Dioxane (20 mL) were added copper(l) iodide (66.59 mg, 0.3500 mmol) and tetrakis(triphenylphosphine) palladium(0) (404.13 mg, 0.3500 mmol) under nitrogen, the mixture was stirred at 100° C. for 16 h. LCMS showed 2-[(6-chloro-2-tributylstannyl-pyrrolo[3,2-c]pyridin-1-yl)methoxy]ethyl-trimethyl-silane was consumed completely and a main peak with desired mass. The mixture was concentrated under vacuum. The residue was purified by flash silica gel chromatography (Biotage, 40 g SepaFlash® Silica Flash Column, Eluent of 0˜10% Ethyl acetate/Petroleum ether gradient @ 35 mL/min, TLC (Petroleum ether:Ethyl acetate=10:1, Rf DP=0.5) to give tert-butyl N-[4-[6-chloro-1-(2-trimethylsilylethoxymethyl)pyrrolo[3,2-c]pyridin-2-yl]-2-pyridyl]-N-(2,2,2-trifluoroethyl)carbamate (1 g, 1.7951 mmol, 51.329% yield) as a yellow oil. ¹H NMR (400 MHz, CDCl₃) δ 8.71 (s, 1H), 8.47 (d, J=5.1 Hz, 1H), 7.97 (s, 1H), 7.45 (s, 1H), 7.37 (dd, J₁=5.1, J₂=1.4 Hz, 1H), 6.83 (s, 1H), 5.45 (s, 2H), 4.85 (q, J=8.7 Hz, 2H), 3.58-3.54 (m, 2H), 1.55 (s, 9H), 0.95-0.91 (m, 2H), -0.03 (s, 9H); LCMS (ESI): m/z 557.2 [M+1]⁺.

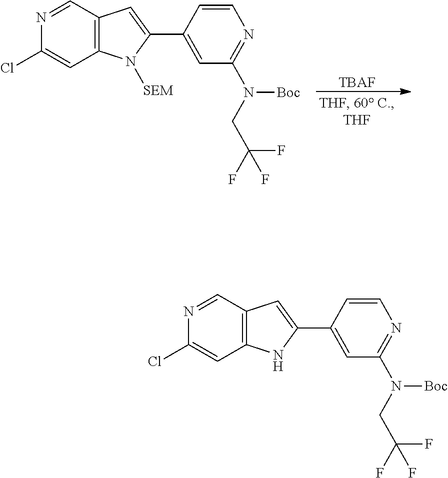 [SMILES: CC(C)(C)OC(=O)N(CC(F)(F)F)c1cc(-c2cc3cnc(Cl)cc3n2COCC[Si](C)(C)C)ccn1 ; "TBAF" ; CC(C)(C)OC(=O)N(CC(F)(F)F)c1cc(-c2cc3cnc(Cl)cc3[nH]2)ccn1]

tert-Butyl (4-(6-chloro-1H-pyrrolo[3,2-c]pyridin-2-yl)pyridin-2-yl)(2,2,2-trifluoroethyl)carbamate. To a solution of tert-butyl N-[4-[6-chloro-1-(2-trimethylsilylethoxymethyl)pyrrolo[3,2-c]pyridin-2-yl]-2-pyridyl]-N-(2,2,2-trifluoroethyl)carbamate (1. g, 1.8 mmol) in THE (10 mL) was added tetra-n-butylammonium fluoride (3.59 mL, 3.59 mmol), the mixture was stirred at 60° C. for 16 h. LCMS showed tert-butyl N-[4-[6-chloro-1-(2-trimethylsilylethoxymethyl)pyrrolo[3,2-c]pyridin-2-yl]-2-pyridyl]-N-(2,2,2-trifluoroethyl)carbamate was consumed completely and a peak (81%) with desired mass. The mixture was concentrated under vacuum. The residue was purified by flash silica gel chromatography (Biotage, 20 g SepaFlash® Silica Flash Column, Eluent of 30˜50% Ethyl acetate/Petroleum ether gradient @ 35 mL/min, TLC (Petroleum ether:Ethyl acetate=3:1, Rf SM=0.4, Rf DP=0.5) to give tert-butyl N-[4-(6-chloro-1H-pyrrolo[3,2-c]pyridin-2-yl)-2-pyridyl]-N-(2,2,2-trifluoroethyl)carbamate (800 mg, 1.6194 mmol, 90.215% yield) as a white solid. ¹H NMR (400 MHz, DMSO-d₆) δ 12.47 (s, 1H), 8.72 (s, 1H), 8.50 (d, J=5.3 Hz, 1H), 8.05 (s, 1H), 7.75 (dd, J₁=5.3, J₂=1.3 Hz, 1H), 7.47 (s, 1H), 7.43 (s, 1H), 4.84 (q, J=9.2 Hz, 2H), 1.48 (s, 9H); LCMS (ESI): m/z 427.0 [M+1]⁺.

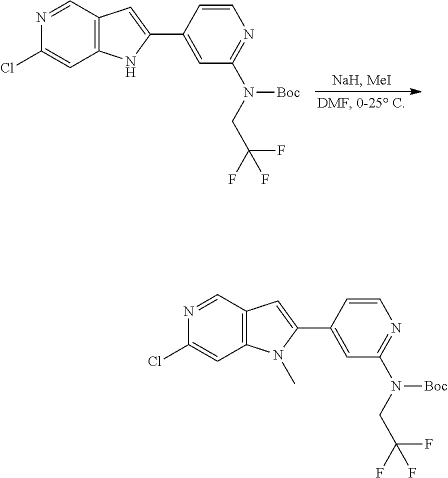

tert-Butyl (4-(6-chloro-1-methyl-1H-pyrrolo[3,2-c]pyridin-2-yl)pyridin-2-yl) (2,2,2-trifluoroethyl)carbamate. To a solution of tert-butyl N-[4-(6-chloro-1H-pyrrolo[3,2-c]pyridin-2-yl)-2-pyridyl]-N-(2,2,2-trifluoroethyl)carbamate (800. mg, 1.87 mmol) in DMF (10 mL) was added sodium hydride (0.06 mL, 3.75 mmol, 60% purity) at 0° C., the mixture was stirred at 25° C. for 0.5 h. Then iodomethane (399.06 mg, 2.81 mmol) was added to the mixture at 0° C., the mixture was stirred at 25° C. for 1 h. TLC (Petroleum ether:Ethyl acetate=3:1, Rf SM=0.3, Rf DP=0.4) indicated starting material was consumed and one major new spot with lower polarity. The mixture was poured into saturated ammonium chloride (100 mL). The aqueous phase was extracted with ethyl acetate (50 mL×2). The combined organic phase was washed with brine (30 mL×3), dried over anhydrous sodium sulfate, filtered and concentrated under vacuum. The residue was purified by flash silica gel chromatography (Biotage, 20 g SepaFlash® Silica Flash Column, Eluent of 30˜60% Ethyl acetate/Petroleum ether gradient @35 mL/min) to give tert-butyl N-[4-(6-chloro-1-methyl-pyrrolo[3,2-c]pyridin-2-yl)-2-pyridyl]-N-(2,2,2-trifluoroethyl)carbamate (300 mg, 0.5784 mmol, 30.861% yield) as a brown solid. ¹H NMR (400 MHz, DMSO-d₆) δ 8.71 (s, 1H), 8.55 (d, J=5.1 Hz, 1H), 7.87 (s, 1H), 7.78 (s, 1H), 7.53 (dd, J₁=5.2, J₂=1.4 Hz, 1H), 6.99 (s, 1H), 4.85 (q, J=9.1 Hz, 2H), 3.82 (s, 3H), 1.48 (s, 9H); LCMS (ESI): m/z 471.1 [M+1]⁺.

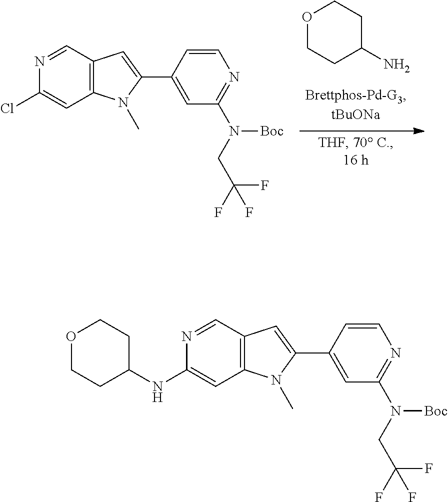 [SMILES: Cn1c(-c2ccnc(N(CC(F)(F)F)C(=O)OC(C)(C)C)c2)cc2cnc(Cl)cc21 ; NC1CCOCC1 ; Cn1c(-c2ccnc(N(CC(F)(F)F)C(=O)OC(C)(C)C)c2)cc2cnc(NC3CCOCC3)cc21]

tert-Butyl (4-(1-methyl-6-((tetrahydro-2H-pyran-4-yl)amino)-1H-pyrrolo[3,2-c]pyridin-2-yl)pyridin-2-yl)(2,2,2-trifluoroethyl)carbamate. To a solution of tert-butyl N-[4-(6-chloro-1-methyl-pyrrolo[3,2-c]pyridin-2-yl)-2-pyridyl]-N-(2,2,2-trifluoroethyl)carbamate (300. mg, 0.6800 mmol) and tetrahydropyran-4-amine (206.5 mg, 2.04 mmol) in THF (3 mL) were added Methanesulfonato(2-dicyclohexylphosphino-3,6-dimethoxy-2,4,6-tri-i-propyl-1,1-biphenyl)(2-amino-1,1-biphenyl-2-yl)palladium(II) (61.69 mg, 0.0700 mmol) and Sodium tert-Butoxide (0.68 mL, 1.36 mmol, 2 M in THF) under nitrogen, the mixture was stirred at 70° C. for 16 h. LCMS showed tert-butyl N-[4-(6-chloro-1-methyl-pyrrolo[3,2-c]pyridin-2-yl)-2-pyridyl]-N-(2,2,2-trifluoroethyl)carbamate was consumed completely and a peak (56%) with desired mass. The mixture was concentrated under vacuum. The residue was purified by flash silica gel chromatography (Biotage, 4 g SepaFlash® Silica Flash Column, Eluent of 30˜60% Ethyl acetate/Petroleum ether gradient @ 20 mL/min) to give tert-butyl N-[4-[1-methyl-6-(tetrahydropyran-4-ylamino)pyrrolo[3,2-c]pyridin-2-yl]-2-pyridyl]-N-(2,2,2-trifluoroethyl)carbamate (230 mg, 0.3553 mmol, 52.216% yield) as a yellow oil. LCMS (ESI): m/z 506.2 [M+1]⁺.

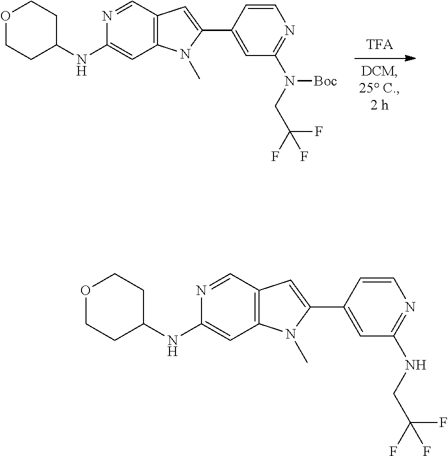 [SMILES: Cn1c(-c2ccnc(N(CC(F)(F)F)C(=O)OC(C)(C)C)c2)cc2cnc(NC3CCOCC3)cc21 ; Cn1c(-c2ccnc(NCC(F)(F)F)c2)cc2cnc(NC3CCOCC3)cc21]

1-Methyl-N-(tetrahydro-2H-pyran-4-yl)-2-(2-((2,2,2-trifluoroethyl)amino)pyridin-4-yl)-1H-pyrrolo[3,2-c]pyridin-6-amine. To a solution of tert-butyl N-[4-[1-methyl-6-(tetrahydropyran-4-ylamino)pyrrolo[3,2-c]pyridin-2-yl]-2-pyridyl]-N-(2,2,2-trifluoroethyl)carbamate (230. mg, 0.4500 mmol) in dichloromethane (10 mL) was added trifluoroacetic acid (2. mL, 0.4500 mmol), the mixture was stirred at 25° C. for 2 h. LCMS showed tert-butyl N-[4-[1-methyl-6-(tetrahydropyran-4-ylamino)pyrrolo[3,2-c]pyridin-2-yl]-2-pyridyl]-N-(2,2,2-trifluoroethyl)carbamate was consumed completely and a peak (88.6%) with desired mass. The mixture was concentrated under vacuum. The residue was purified by prep-HPLC (column: Unisil 3-100 C18 Ultra 150*50 mm*3 um; mobile phase: [water(0.225% FA) -ACN];B %: 13%-33%,10 min), then dried by lyophilization to give 1-methyl-N-tetrahydropyran-4-yl-2-[2-(2,2,2-trifluoroethylamino)-4-pyridyl]pyrrolo[3,2-c]pyridin-6-amine (102.47 mg, 0.2520 mmol, 55.387% yield) as a yellow solid. ¹H NMR (400 MHz, DMSO-d₆) δ 8.46 (s, 1H), 8.14-8.13 (m, 1H), 7.34 (t, J=6.5 Hz, 1H), 6.84 (dd, J₁=5.3, J₂=1.3 Hz, 1H), 6.81 (s, 1H), 6.73 (s, 2H), 4.24-4.20 (m, 2H), 3.92-3.89 (m, 3H), 3.67 (s, 3H), 3.48-3.34 (m, 2H), 1.96-1.92 (m, 2H), 1.52-1.46 (m, 2H); LCMS (ESI): m/z 406.2 [M+1]⁺.

### Example 40: N-Isopropyl-1-methyl-2-[2-(2,2,2-trifluoroethylamino)pyrimidin-4-yl]pyrrolo[3,2-c]pyridin-6-amine

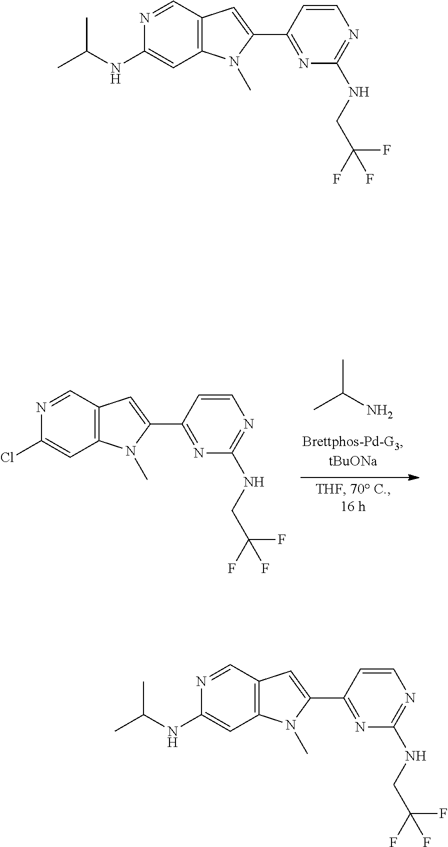

N-Isopropyl-1-methyl-2-[2-(2,2,2-trifluoroethylamino)pyrimidin-4-yl]pyrrolo[3,2-c]pyridin-6-amine. To a mixture of 4-(6-chloro-1-methyl-pyrrolo[3,2-c]pyridin-2-yl)-N-(2,2,2-trifluoroethyl)pyrimidin-2-amine (250 mg, 0.73 mmol) in tetrahydrofuran (4 mL) was added propan-2-amine (129 mg, 2.19 mmol), methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (132 mg, 0.15 mmol) and sodium tert-butoxide (0.73 mL, 1.46 mmol, 2 M in THF). The mixture was stirred at 70° C. for 16 h under nitrogen. LCMS showed a peak (62%) with desired mass. The reaction mixture was filtered and the filtrate was concentrated. The residue obtained was purified by prep-HPLC (column: Phenomenex Luna C18 150*40 mm*15 um; mobile phase: [water (0.225% FA)-ACN]; B %: 12%-42%, 10 min) followed by lyophilization to give N-isopropyl-1-methyl-2-[2-(2,2,2-trifluoroethylamino)pyrimidin-4-yl]pyrrolo[3,2-c]pyridin-6-amine (101.48 mg, 0.28 mmol, 37.84% yield) as a yellow solid. ¹H NMR (400 MHz, DMSO-d₆) δ 8.40 (s, 1H), 8.33 (d, J=5.0 Hz, 1H), 7.81 (t, J=6.2 Hz, 1H), 7.18-7.15 (m, 2H), 6.32 (s, 1H), 5.89 (d, J=8.0 Hz, 1H), 4.24-4.12 (m, 2H), 4.03-3.91 (m, 4H), 1.18 (d, J=6.2 Hz, 6H); MS (ESI): m/z 365.2 [M+1]⁺.

### Example 41: N-tert-Butyl-1-methyl-2-[2-(2,2,2-trifluoroethylamino)pyrimidin-4-yl]pyrrolo[3,2-c]pyridin-6-amine

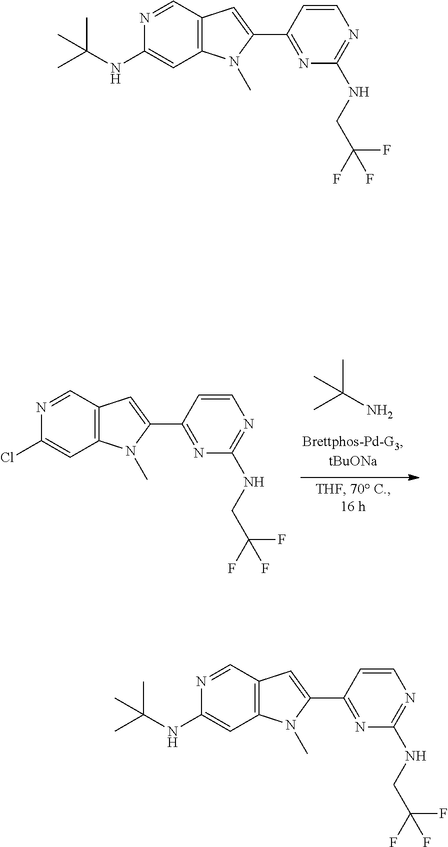

N-tert-Butyl-1-methyl-2-[2-(2,2,2-trifluoroethylamino)pyrimidin-4-yl]pyrrolo[3,2-c]pyridin-6-amine. To a mixture of 4-(6-chloro-1-methyl-pyrrolo[3,2-c]pyridin-2-yl)-N-(2,2,2-trifluoroethyl)pyrimidin-2-amine (250. mg, 0.73 mmol) in tetrahydrofuran (5 mL) was added 2-methylpropan-2-amine (160.53 mg, 2.19 mmol), Methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (132.91 mg, 0.15 mmol) and sodium tert-butoxide (0.73 mL, 1.46 mmol, 2 M in THF). The mixture was stirred at 70° C. for 16 h under nitrogen. LCMS showed a peak (50%) with desired mass. The reaction mixture was filtered and the filtrate was concentrated under vacuum. The residue was purified by prep-HPLC (column: Phenomenex Luna C18 150*40 mm*15 um; mobile phase: [water(0.225% FA)-ACN];B %: 12%-42%,10 min) followed by lyophilization to give N-tert-butyl-1-methyl-2-[2-(2,2,2-trifluoroethylamino)pyrimidin-4-yl]pyrrolo[3,2-c]pyridin-6-amine (95.8 mg, 0.25 mmol, 34.57% yield) as a yellow solid. ¹H NMR (400 MHz, DMSO-d₆) δ 8.41 (d, J=0.6 Hz, 1H), 8.32 (d, J=5.2 Hz, 1H), 8.14 (s, 0.2H), 7.80 (t, J=6.6 Hz, 1H), 7.17 (d, J=5.4 Hz, 1H), 7.15 (s, 1H), 6.42 (s, 1H), 4.21-4.15 (m, 2H), 3.94 (s, 3H), 1.41 (s, 9H); MS (ESI): m/z 379.2 [M+1]⁺.

### Example 42: N-Tetrahydropyran-4-yl-2-[2-(3,3,3-trifluoropropyl)pyrimidin-4-yl]-1H-pyrrolo[3,2-c]pyridin-6-amine

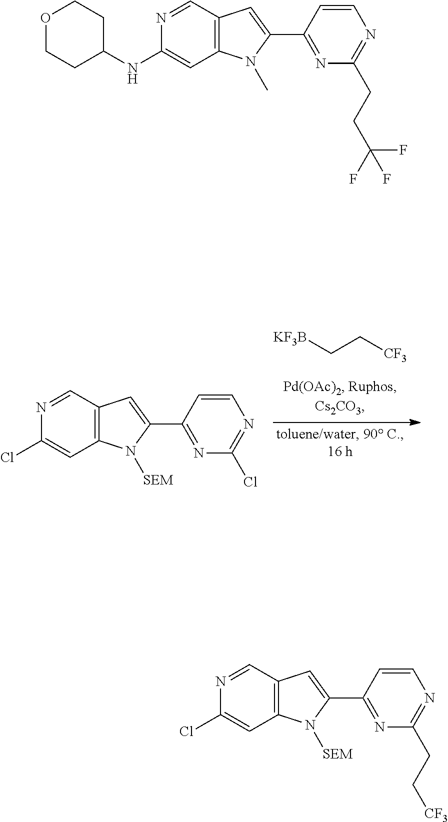

2-[[6-Chloro-2-[2-(3,3,3-trifluoropropyl)pyrimidin-4-yl]pyrrolo[3,2-c]pyridin-1-yl]methoxy]ethyl-trimethyl-silane. To a mixture of 2-[[6-chloro-2-(2-chloropyrimidin-4-yl)pyrrolo[3,2-c]pyridin-1-yl]methoxy]ethyl-trimethyl-silane (1200. mg, 3.04 mmol) in toluene (10 mL) and water (2 mL) was added potassium; trifluoro(3,3,3-trifluoropropyl)boranuide (928.59 mg, 4.55 mmol), cesium carbonate (2966.79 mg, 9.11 mmol) and (2-Dicyclohexylphosphino-2,6-amino-1,1-biphenyl)]palladium(II) methanesulfonate (548.77 mg, 0.61 mmol). The mixture was stirred at 90° C. for 16 h. LCMS showed a peak (7%) with desired mass. The residue was purified by flash silica gel chromatography (20 g SepaFlash® Silica Flash Column, Eluent of 20˜30% Ethyl acetate/Petroleum ether gradient) to give 2-[[6-chloro-2-[2-(3,3,3-trifluoropropyl)pyrimidin-4-yl]pyrrolo[3,2-c]pyridin-1-yl]methoxy]ethyl-trimethyl-silane (300 mg) as a yellow solid. LCMS showed a peak (87%) with desired mass. The residue was purified by prep-HPLC (column: Phenomenex Luna C18 150*25 mm*10 um; mobile phase: [water (0.225% TFA)-ACN]; B %: 67%-97%, 25 min) followed by lyophilization to give 2-[[6-chloro-2-[2-(3,3,3-trifluoropropyl)pyrimidin-4-yl]pyrrolo[3,2-c]pyridin-1-yl]methoxy]ethyl-trimethyl-silane (200 mg, 0.44 mmol, 14.41% yield) as a yellow solid.

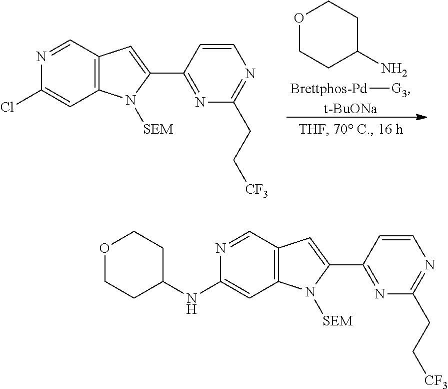

N-Tetrahydropyran-4-yl-2-[2-(3,3,3-trifluoropropyl)pyrimidin-4-yl]-1-(2-trimethylsilylethoxymethyl)pyrrolo[3,2-c]pyridin-6-amine. To a mixture of 2-[[6-chloro-2-[2-(3,3,3-trifluoropropyl)pyrimidin-4-yl]pyrrolo[3,2-c]pyridin-1-yl]methoxy]ethyl-trimethyl-silane (200. mg, 0.44 mmol) in tetrahydrofuran (3 mL) was added tetrahydropyran-4-amine (132.81 mg, 1.31 mmol), Methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (79.51 mg, 0.09 mmol) and sodium tert-butoxide (0.44 mL, 0.88 mmol, 2 M in THF). The mixture was stirred at 70° C. for 16 h under nitrogen. LCMS showed a peak (73%) with desired mass. The reaction mixture was filtered and the filtrate was concentrated. The residue was purified by flash silica gel chromatography (10 g SepaFlash® Silica Flash Column, Eluent of 40˜60% Ethyl acetate/Petroleum ether) to give N-tetrahydropyran-4-yl-2-[2-(3,3,3-trifluoropropyl)pyrimidin-4-yl]-1-(2-trimethylsilylethoxymethyl)pyrrolo[3,2-c]pyridin-6-amine (200 mg, 0.38 mmol, 86.29% yield) as a yellow solid. MS (ESI): m/z 522.2 [M+1]⁺.

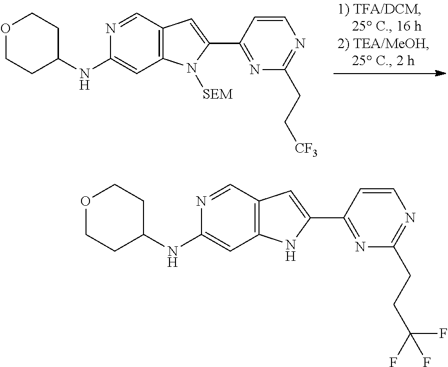

N-Tetrahydropyran-4-yl-2-[2-(3,3,3-trifluoropropyl)pyrimidin-4-yl]-1H-pyrrolo[3,2-c]pyridin-6-amine. To a mixture of N-tetrahydropyran-4-yl-2-[2-(3,3,3-trifluoropropyl)pyrimidin-4-yl]-1-(2-trimethylsilylethoxymethyl)pyrrolo[3,2-c]pyridin-6-amine (200 mg, 0.38 mmol) in dichloromethane (3 mL) was added Trifluoroacetic acid (3. mL, 0.38 mmol). The mixture was stirred at 25° C. for 16 h. LCMS showed a peak (97%) with desired mass. After that, the solvent was removed under vacuum. The residue was dissolved in methanol (5 mL), the triethylamine (0.05 mL, 0.3800 mmol) was added to the solution, stirred at 25° C. for 2 h. LCMS showed starting material was consumed completely and a major peak (97%) with desired mass. The residue was purified by prep-HPLC (column: Phenomenex Luna C18 150*25 mm*10 um; mobile phase: [water (0.225% FA)-ACN]; B %: 10%-43%, 10 min) followed by lyophilization to give N-tetrahydropyran-4-yl-2-[2-(3,3,3-trifluoropropyl)pyrimidin-4-yl]-1H-pyrrolo[3,2-c]pyridin-6-amine (111.76 mg, 0.28 mmol, 73.88% yield) as a yellow solid. ¹H NMR (400 MHz, DMSO-d₆) δ 11.76 (s, 1H), 8.76 (d, J=5.4 Hz, 1H), 8.54 (s, 1H), 8.14 (s, 0.2H), 7.89 (d, J=5.3 Hz, 1H), 7.46 (s, 1H), 6.91 (s, 1H), 6.62 (s, 1H), 3.96-3.87 (m, 2H), 3.81-3.80 (m, 1H), 3.46-3.41 (m, 2H), 3.20-3.16 (m, 2H), 3.01-2.91 (m, 2H), 1.94-1.90 (m, 2H), 1.55-1.42 (m, 2H); MS (ESI): m/z 392.1 [M+1]⁺.

### Example 43: 1-Methyl-N-[rac-(3R)-tetrahydropyran-3-yl]-2-[2-(2,2,2-trifluoroethylamino)pyrimidin-4-yl]pyrrolo[3,2-c]pyridin-6-amine

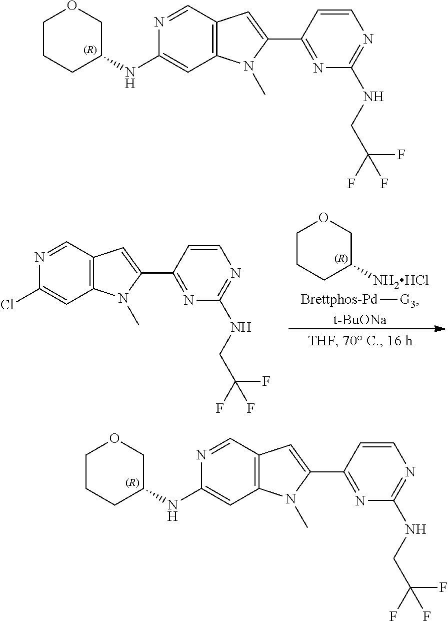

1-Methyl-N-[rac-(3R)-tetrahydropyran-3-yl]-2-[2-(2,2,2-trifluoroethylamino)pyrimidin-4-yl]pyrrolo[3,2-c]pyridin-6-amine. To 4-(6-chloro-1-methyl-pyrrolo[3,2-c]pyridin-2-yl)-N-(2,2,2-trifluoroethyl)pyrimidin-2-amine (180 mg, 0.53 mmol) in tetrahydrofuran (5 mL) was added rac-(3R)-tetrahydropyran-3-amine hydrochloride (217 mg, 1.58 mmol), methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (96 mg, 0.11 mmol) and sodium tert-butoxide (0.53 mL, 1.05 mmol, 2 M in THF). The reaction mixture was stirred at 70° C. for 16 h under nitrogen. The mixture was filtered and concentrated, then purified by prep-HPLC (column: Phenomenex Luna C18 150*40 mm*15 um; mobile phase: [water(0.225% FA)-ACN]; B %: 10%-40%, 10 min) followed by lyophilization to give 1-methyl-N-[rac-(3R)-tetrahydropyran-3-yl]-2-[2-(2,2,2-trifluoroethylamino)pyrimidin-4-yl]pyrrolo[3,2-c]pyridin-6-amine (104 mg, 0.25 mmol, 48.1% yield, formic acid) as a yellow solid. tH NMR (400 MHz, DMSO-d₆) δ 8.39 (d, J=0.6 Hz, 1H), 8.33 (d, J=5.4 Hz, 1H), 8.16 (s, 1H), 7.81 (t, J=6.8 Hz, 1H), 7.18-7.15 (m, 2H), 6.41 (s, 1H), 6.03 (d, J=8.2 Hz, 1H), 4.23-4.14 (m, 2H), 3.96 (s, 3H), 3.93-3.92 (m, 1H), 3.80-3.85 (m, 1H), 3.76-3.72 (m, 1H), 3.334-3.33 (m, 1H), 3.13-3.09 (m, 1H), 2.01-1.96 (m, 1H), 1.76-1.67 (m, 1H), 1.62-1.48 (m, 2H); MS (ESI): m/z 407.1 [M+1]⁺.

### Example 44: 2-(6-(Methyl(2,2,2-trifluoroethyl)amino)pyrimidin-4-yl)-N-(tetrahydro-2H-pyran-4-yl)-1H-pyrrolo[3,2-c]pyridin-6-amine

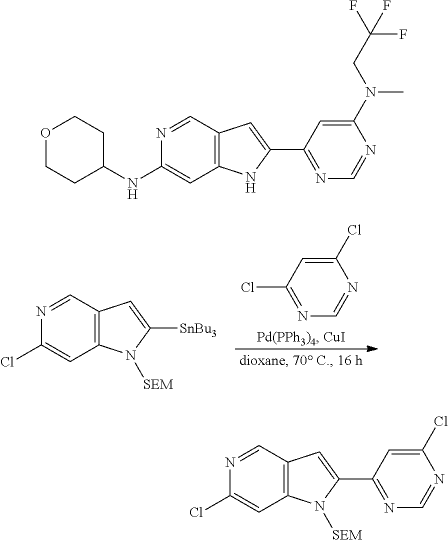

6-Chloro-2-(6-chloropyrimidin-4-yl)-1-((2-(trimethylsilyl)ethoxy)methyl)-1H-pyrrolo[3,2-c]pyridine. To a solution of 2-[(6-chloro-2-tributylstannyl-pyrrolo[3,2-c]pyridin-1-yl)methoxy]ethyl-trimethyl-silane (3. g, 5.25 mmol) and 4,6-dichloropyrimidine (1172.27 mg, 7.87 mmol) in 1,4-Dioxane (3 mL) were added tetrakis[triphenylphosphine]palladium(0) (606.18 mg, 0.5200 mmol) and copper iodide (99.91 mg, 0.5200 mmol) under nitrogen, the mixture was stirred at 70° C. for 16 h. LCMS showed 2-[(6-chloro-2-tributylstannyl-pyrrolo[3,2-c]pyridin-1-yl)methoxy]ethyl-trimethyl-silane was consumed completely and a peak with desired mass. The mixture was concentrated under vacuum. The residue was purified by flash silica gel chromatography (Biotage, 40 g SepaFlash® Silica Flash Column, Eluent of 30-60% Ethyl acetate/Petroleum ether gradient @ 35 mL/min) to give 2-[[6-chloro-2-(6-chloropyrimidin-4-yl)pyrrolo[3,2-c]pyridin-1-yl]methoxy]ethyl-trimethyl-silane (900 mg, 2.27 mmol, 43.3% yield) as a yellow oil. ¹H NMR (400 MHz, DMSO-d₆) δ 9.37 (d, J=1.1 Hz, 1H), 9.04 (d, J=0.6 Hz, 1H), 8.56 (d, J=1.1 Hz, 1H), 8.16 (s, 1H), 7.94 (s, 1H), 6.38 (s, 2H), 3.60 (t, J=7.7 Hz, 2H), 0.92 (t, J=7.8 Hz, 2H), 0.00 (s, 9H); MS (ESI): m/z 395.0 [M+1]⁺.

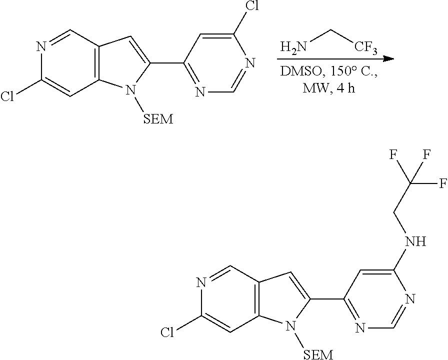 [SMILES: C[Si](C)(C)CCOCn1c(-c2cc(Cl)ncn2)cc2cnc(Cl)cc21 ; NCC(F)(F)F ; C[Si](C)(C)CCOCn1c(-c2cc(NCC(F)(F)F)ncn2)cc2cnc(Cl)cc21]

6-(6-Chloro-1-((2-(trimethylsilyl)ethoxy)methyl)-1H-pyrrolo[3,2-c]pyridin-2-yl)-N-(2,2,2-trifluoroethyl)pyrimidin-4-amine. To a solution of 2-[[6-chloro-2-(6-chloropyrimidin-4-yl)pyrrolo[3,2-c]pyridin-1-yl]methoxy]ethyl-trimethyl-silane (900. mg, 2.28 mmol) in DMSO (4 mL) was added 2,2, 2-trifluoroethanamine (3.57 mL, 45.53 mmol) under nitrogen, the mixture was stirred at 150° C. for 2 h under microwave. LCMS showed 2-[[6-chloro-2-(6-chloropyrimidin-4-yl)pyrrolo[3,2-c]pyridin-1-yl]methoxy]ethyl-trimethyl-silane was consumed completely and a major peak with desired mass. The mixture was poured into water (50 mL). The aqueous phase was extracted with ethyl acetate (40 mL×3). The combined organic phase was washed with brine (40 mL×5), dried over anhydrous sodium sulfate, filtered and concentrated under vacuum. The residue was purified by silica gel chromatography (200-300 mesh silica gel, Petroleum ether/Ethyl acetate=100/1, 3/1, TLC: Petroleum ether/Ethyl acetate=1/1, RF=0.6) to give 6-[6-chloro-1-(2-trimethylsilylethoxymethyl)pyrrolo[3,2-c]pyridin-2-yl]-N-(2,2,2-trifluoroethyl)pyrimidin-4-amine (650 mg, 1.27 mmol, 56.0% yield) was a yellow solid. ¹H NMR (400 MHz, DMSO-d₆) δ 8.75 (d, J=0.8 Hz, 1H), 8.63 (s, 1H), 8.20 (s, 1H), 7.85 (s, 1H), 7.22 (s, 1H), 7.09 (s, 1H), 6.10 (s, 2H), 4.34-4.25 (m, 2H), 3.30 (t, J=7.9 Hz, 2H), 0.66 (t, J=7.9 Hz, 2H), -0.23 (s, 8H); MS (ESI): m/z 458.1 [M+1]⁺.

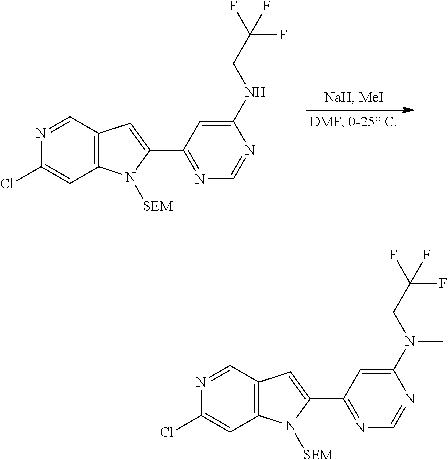 [SMILES: C[Si](C)(C)CCOCn1c(-c2cc(NCC(F)(F)F)ncn2)cc2cnc(Cl)cc21 ; CN(CC(F)(F)F)c1cc(-c2cc3cnc(Cl)cc3n2COCC[Si](C)(C)C)ncn1]

6-(6-Chloro-1-((2-(trimethylsilyl)ethoxy)methyl)-1H-pyrrolo[3,2-c]pyridin-2-yl)-N-methyl-N-(2,2,2-trifluoroethyl)pyrimidin-4-amine. To a solution of 6-[6-chloro-1-(2-trimethylsilylethoxymethyl)pyrrolo[3,2-c]pyridin-2-yl]-N-(2,2,2-trifluoroethyl)pyrimidin-4-amine (600 mg, 1.31 mmol) in DMF (10 mL) was added sodium hydride (104.81 mg, 2.62 mmol, 60% purity) at 0° C., the mixture was stirred at 25° C. for 0.5 h. Then iodomethane (278.95 mg, 1.97 mmol) was added to the mixture at 0° C. and stirred at 25° C. for 1 h. TLC (Petroleum ether:Ethyl acetate=2:1, Rf SM=0.3, Rf DP=0.4) indicated starting material was consumed and one major new spot with lower polarity. The mixture was poured into saturated ammonium chloride aqueous (100 mL). The aqueous phase was extracted with ethyl acetate (50 mL×2). The combined organic phase was washed with brine (30 mL×3), dried over anhydrous sodium sulfate, filtered and concentrated under vacuum. The residue was purified by silica gel chromatography (200-300 mesh silica gel, Petroleum ether/Ethyl acetate=5/1, 1/1) to give 6-[6-chloro-1-(2-trimethylsilylethoxymethyl)pyrrolo[3,2-c]pyridin-2-yl]-N-methyl-N-(2,2,2-trifluoroethyl)pyrimidin-4-amine (450 mg, 0.95 mmol, 72.7% yield) as a yellow oil. ¹H NMR (400 MHz, DMSO-d₆) δ 8.76 (s, 1H), 8.70 (d, J=1.0 Hz, 1H), 7.87 (s, 1H), 7.44 (s, 1H), 7.35 (s, 1H), 6.14 (s, 2H), 4.66-4.57 (m, 2H), 3.34-3.34 (m, 2H), 3.22 (s, 3H), 0.67 (t, J=7.9 Hz, 2H), -0.23 (m, 9H); MS (ESI): m/z 472.1 [M+1]⁺.

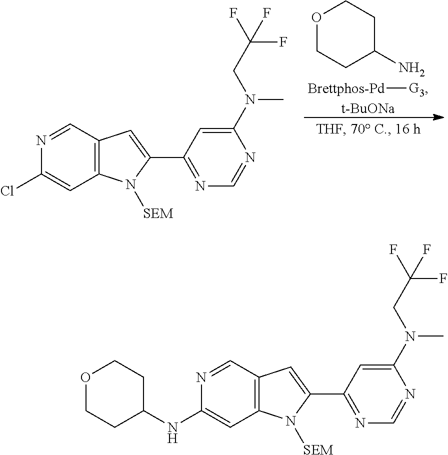

2-(6-(Methyl(2,2,2-trifluoroethyl)amino)pyrimidin-4-yl)-N-(tetrahydro-2H-pyran-4-yl)-1-((2-(trimethylsilyl)ethoxy)methyl)-1H-pyrrolo[3,2-c]pyridin-6-amine. To a solution of 6-[6-chloro-1-(2-trimethylsilylethoxymethyl)pyrrolo[3,2-c]pyridin-2-yl]-N-methyl-N-(2,2,2-trifluoroethyl)pyrimidin-4-amine (450. mg, 0.9500 mmol) and tetrahydropyran-4-amine (289.32 mg, 2.86 mmol) in THF (2 mL) were added Methanesulfonato(2-dicyclohexylphosphino-3, 6-dimethoxy-2,4,6-tri-i-propyl-1,1-biphenyl)(2-amino-1,1-biphenyl-2-yl)palladium(II) (86.43 mg, 0.1000 mmol) and Sodium tert-Butoxide (0.95 mL, 1.91 mmol, 2 M in THF) under nitrogen, the mixture was stirred at 60° C. for 16 h. LCMS showed 6-[6-chloro-1-(2-trimethylsilyl ethoxymethyl)pyrrolo[3,2-c]pyridin-2-yl]-N-methyl-N-(2,2,2-trifluoroethyl)pyrimidin-4-amine was consumed completely. TLC (Petroleum ether:Ethyl acetate=1:1, Rf SM=0.8, Rf DP=0.2) indicated starting material was consumed and one major new spot with larger polarity. The mixture was concentrated under vacuum. The residue was purified by flash silica gel chromatography (Biotage, 12 g SepaFlash® Silica Flash Column, Eluent of 30˜70% Ethyl acetate/Petroleum ether gradient @ 30 mL/min) to give 2-[6-[methyl(2,2,2-trifluoroethyl)amino]pyrimidin-4-yl]-N-tetrahydropyran-4-yl-1-(2-trimethylsilylethoxymethyl)pyrrolo[3,2-c]pyridin-6-amine (430 mg, 0.80 mmol, 83.9% yield) as a yellow oil. ¹H NMR (400 MHz, CDCl₃) δ 8.68 (d, J=1.1 Hz, 1H), 8.47 (d, J=0.8 Hz, 1H), 6.94 (s, 2H), 6.38 (s, 1H), 5.94 (s, 2H), 4.40 (q, J=8.8 Hz, 2H), 4.04-4.01 (m, 2H), 3.61-3.58 (m, 1H), 3.48-3.49 (m, 2H), 3.46-3.44 (m, 2H), 3.21 (s, 3H), 2.11-2.08 (m, 2H), 1.61-1.52 (m, 2H), 0.84-0.80 (m, 2H), -0.12 (s, 9H); MS (ESI): m/z 537.3 [M+1]⁺.

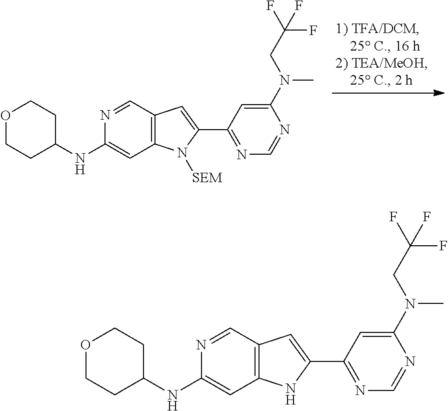 [SMILES: CN(CC(F)(F)F)c1cc(-c2cc3cnc(NC4CCOCC4)cc3n2COCC[Si](C)(C)C)ncn1 ; CN(CC(F)(F)F)c1cc(-c2cc3cnc(NC4CCOCC4)cc3[nH]2)ncn1]

2-(6-(Methyl(2,2,2-trifluoroethyl)amino)pyrimidin-4-yl)-N-(tetrahydro-2H-pyran-4-yl)-1H-pyrrolo[3,2-c]pyridin-6-amine. To a solution of 2-[6-[methyl(2,2,2-trifluoroethyl)amino]pyrimidin-4-yl]-N-tetrahydropyran-4-yl-1-(2-trimethylsilylethoxymethyl)pyrrolo[3,2-c]pyridin-6-amine (300. mg, 0.5600 mmol) in dichloromethane (10 mL) was added trifluoroacetic acid (10. mL, 0.5600 mmol), the mixture was stirred at 25° C. for 16 h. LCMS showed 2-[6-[methyl(2,2,2-trifluoroethyl)amino]pyrimidin-4-yl]-N-tetrahydropyran-4-yl-1-(2-trimethylsilylethoxymethyl)pyrrolo[3,2-c]pyridin-6-amine was consumed completely and a peak (90%) with desired mass of intermediate. The mixture was concentrated under oil pump. The residue in methanol (10 mL) was added triethylamine (10. mL, 0.5600 mmol) was stirred at 25° C. for 2 h. LCMS showed a main peak with desired mass. The mixture was concentrated under vacuum. The residue was purified by prep-HPLC (column: Unisil 3-100 C18 Ultra 150*50 mm*3 um; mobile phase: [water (0.225% FA) -ACN]; B %: 30%-50%, 10 min), then dried by lyophilization to give 2-[6-[methyl(2,2,2-trifluoroethyl)amino]pyrimidin-4-yl]-N-tetrahydropyran-4-yl-1H-pyrrolo[3,2-c]pyridin-6-amine (151.9 mg, 0.37 mmol, 66.0% yield, Formic acid) as a yellow solid. ¹H NMR (400 MHz, DMSO-d₆) δ 11.92 (s, 1H), 8.65 (d, J=1.0 Hz, 1H), 8.53 (s, 1H), 8.14 (s, 1H), 7.42-7.39 (m, 2H), 7.02 (s, 1H), 6.65 (s, 1H), 4.60 (q, J=9.5 Hz, 2H), 3.91-3.89 (m, 2H), 3.80-3.78 (m, 1H), 3.46-3.43 (m, 2H), 3.22 (s, 3H), 1.93-1.90 (m, 2H), 1.49-1.46 (m, 2H); MS (ESI): m/z 407.2 [M+1]⁺.

### Example 45: 2-(2,6-Dimethylpyrimidin-4-yl)-N-(tetrahydro-2h-pyran-4-yl)-1H-pyrrolo[3,2-c]pyridin-6-amine

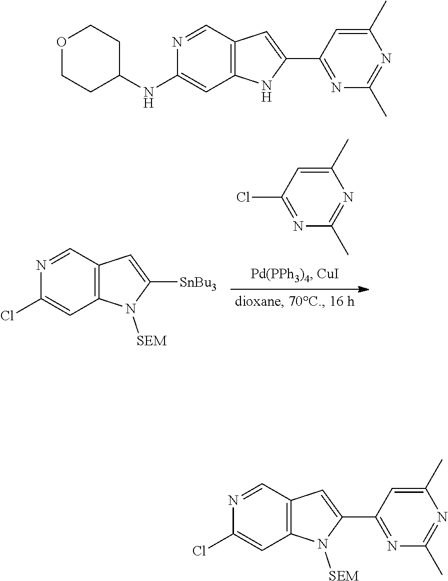

6-Chloro-2-(2,6-dimethylpyrimidin-4-yl)-1-((2-(trimethylsilyl)ethoxy)methyl)-1H-pyrrolo[3,2-c]pyridine. To a solution of 2-[(6-chloro-2-tributylstannyl-pyrrolo[3,2-c]pyridin-1-yl)methoxy]ethyl-trimethyl-silane (5. g, 8.74 mmol) and 4-chloro-2,6-dimethyl-pyrimidine (1.87 g, 13.11 mmol) in 1,4-Dioxane (50 mL) were added tetrakis[triphenylphosphine]palladium(0) (1.01 g, 0.8700 mmol) and copper iodide (166.51 mg, 0.8700 mmol) under nitrogen, the mixture was stirred at 70° C. for 16 h. LCMS showed 4-chloro-2,6-dimethyl-pyrimidine was consumed and a peak (20%) with desired mass. The mixture was concentrated under vacuum. The residue was purified by flash silica gel chromatography (Biotage, 40 g SepaFlash® Silica Flash Column, Eluent of 20˜70% Ethyl acetate/Petroleum ether gradient @ 30 mL/min, TLC (Petroleum ether:Ethyl acetate=3:1, Rf DP=0.5) to give 2-[[6-chloro-2-(2,6-dimethylpyrimidin-4-yl)pyrrolo[3,2-c]pyridin-1-yl]methoxy]ethyl-trimethyl-silane (0.85 g, 2.185 mmol, 24.9% yield) as a yellow solid. ¹H NMR (400 MHz, DMSO-d₆) δ 8.76 (d, J=0.6 Hz, 1H), 7.87 (s, 1H), 7.76 (s, 1H), 7.46 (d, J=0.6 Hz, 1H), 6.19 (s, 2H), 3.39-3.34 (m, 2H), 2.64 (s, 3H), 2.49 (s, 3H), 0.69 (t, J=7.8 Hz, 2H), -0.23 (m, 9H); MS (ESI): m/z 388.9 [M+1]⁺.

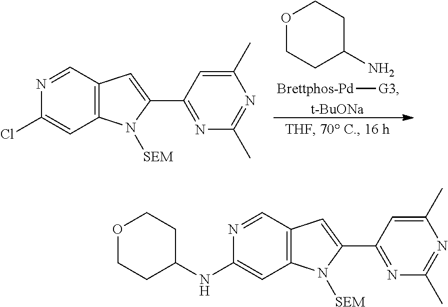

2-(2,6-dimethylpyrimidin-4-yl)-N-(tetrahydro-2H-pyran-4-yl)-1-((2-(trimethyl silyl)ethoxy)methyl)-1H-pyrrolo[3,2-c]pyridin-6-amine. To a solution of 2-[[6-chloro-2-(2,6-dimethylpyrimidin-4-yl)pyrrolo[3,2-c]pyridin-1-yl]methoxy]ethyl-trimethyl-silane (300.0 mg, 0.7700 mmol) and tetrahydropyran-4-amine (234.04 mg, 2.31 mmol) in THF (10 mL) were added Methanesulfonato(2-dicyclohexylphosphino-3,6-dimethoxy-2,4,6-tri-i-propyl-1,1-biphenyl)(2-amino-1,1-biphenyl-2-yl)palladium(II) (69.92 mg, 0.0800 mmol) and Sodium tert-Butoxide (0.77 mL, 1.54 mmol, 2 M in THF), the mixture was stirred at 70° C. for 16 h under nitrogen. LCMS showed 2-[[6-chloro-2-(2,6-dimethyl pyrimidin-4-yl)pyrrolo[3,2-c]pyridin-1-yl]methoxy]ethyl-trimethyl-silane was consumed completely and a peak (58%) with desired mass. The mixture was concentrated under vacuum. The residue was purified by flash silica gel chromatography (Biotage, 12 g SepaFlash® Silica Flash Column, Eluent of 30˜100% Ethyl acetate/Petroleum ether gradient @ 30 mL/min, TLC (Petroleum ether:Ethyl acetate=1:1, Rf=0.3) to give 2-(2,6-dimethylpyrimidin-4-yl)-N-tetrahydropyran-4-yl-1-(2-trimethylsilylethoxymethyl)pyrrolo[3,2-c]pyridin-6-amine (290 mg, 0.5197 mmol, 67.385% yield) as a yellow oil. ¹H NMR (400 MHz, CDCl₃) δ 8.48 (d, J=0.9 Hz, 1H), 7.35 (s, 1H), 7.02 (d, J=0.8 Hz, 1H), 6.40 (s, 1H), 6.07 (s, 2H), 4.05-4.01 (m, 2H), 3.81-3.78 (m, 1H), 3.62-3.56 (m, 2H), 3.52-3.48 (m, 2H), 2.73 (s, 3H), 2.54 (s, 3H), 2.12-2.08 (m, 2H), 1.65-1.50 (m, 2H), 0.85-0.81 (m, 2H), −0.09-0.11 (s, 9H); MS (ESI): m/z 454.1 [M+1]⁺.

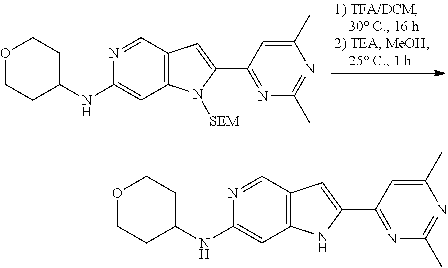 [SMILES: Cc1cc(-c2cc3cnc(NC4CCOCC4)cc3n2COCC[Si](C)(C)C)nc(C)n1 ; Cc1cc(-c2cc3cnc(NC4CCOCC4)cc3[nH]2)nc(C)n1]

2-(2,6-dimethylpyrimidin-4-yl)-N-(tetrahydro-2H-pyran-4-yl)-1H-pyrrolo[3,2-c]pyridin-6-amine. To a solution of 2-(2,6-dimethylpyrimidin-4-yl)-N-tetrahydropyran-4-yl-1-(2-trimethylsilylethoxymethyl)pyrrolo[3,2-c]pyridin-6-amine (290. mg, 0.6400 mmol) in dichloromethane (10 mL) were added trifluoroacetic acid (10. mL, 0.6400 mmol), the mixture was stirred at 30° C. for 16 h. LCMS showed 2-(2,6-dimethylpyrimidin-4-yl)-N-tetrahydropyran-4-yl-1-(2-trimethylsilylethoxymethyl)pyrrolo[3,2-c]pyridin-6-amine was consumed completely and a peak (88%) with desired mass. The mixture was concentrated under oil pump. The residue in methanol (10 mL) was added triethylamine (10. mL, 0.6400 mmol) was stirred at 25° C. for 1 h. LCMS showed a peak (87%) with desired mass. The mixture was concentrated under vacuum. The residue was purified by prep-HPLC (column: Phenomenex luna C18 150*40 mm*15 um; mobile phase: [water (0.225% FA) -ACN]; B %: 1%-30%, 10 min), then dried by lyophilization to give 2-(2,6-dimethylpyrimidin-4-yl)-N-tetrahydropyran-4-yl-1H-pyrrolo[3,2-c]pyridin-6-amine (84.83 mg, 0.25 mmol, 39.8% yield) as a yellow solid. Additional 2.11 mg for delivery. ¹H NMR (400 MHz, MeOD-d₄) δ 8.43 (d, J=0.9 Hz, 1H), 7.66 (s, 1H), 7.37 (d, J=0.7 Hz, 1H), 6.67 (s, 1H), 4.04-3.99 (m, 2H), 3.62-3.59 (m, 1H), 3.59-3.56 (m, 2H), 2.70 (s, 3H), 2.54 (s, 3H), 2.06-2.02 (m, 2H), 1.63-1.58 (m, 2H); MS (ESI): m/z 324.1 [M+1]⁺.

### Example 46: 1-Methyl-N-(tetrahydro-2H-pyran-4-yl)-2-(6-(trifluoromethyl)pyrimidin-4-yl)-1H-pyrrolo[3,2-c]pyridin-6-amine

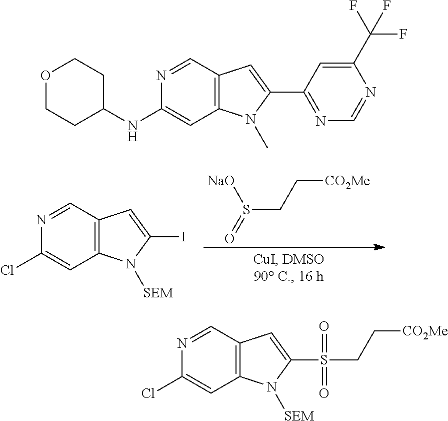

Methyl 3-((6-chloro-1-((2-(trimethylsilyl)ethoxy)methyl)-1H-pyrrolo[3,2-c]pyridin-2-yl)sulfonyl)propanoate. To a mixture of (3-methoxy-3-oxo-propyl)sulfinyloxysodium (0.5 g, 2.87 mmol) in DMSO (5 mL) was added 6-chloro-2-iodo-1-((2-(trimethylsilyl)ethoxy)methyl)-1H-pyrrolo[3,2-c]pyridine (1.53 g, 3.73 mmol) and cuprous iodide (1.64 g, 8.61 mmol). The mixture was stirred at 90° C. for 16 h. LCMS showed 6-chloro-2-iodo-1-((2-(trimethylsilyl)ethoxy)methyl)-1H-pyrrolo[3,2-c]pyridine (40%) completely and a peak (40%) with desired mass. The residue was poured into water (30 mL). The aqueous phase was extracted with ethyl acetate (30 mL×3). The combined organic phase was washed with brine (10 mL×2), dried over anhydrous sodium sulfate, filtered and concentrated under vacuum. The residue was purified by flash silica gel chromatography (Biotage, 20 g SepaFlash® Silica Flash Column, eluent of 30˜60% ethyl acetate/petroleum ether gradient @ 30 ml/min) to give methyl 3-((6-chloro-1-((2-(trimethylsilyl)ethoxy)methyl)-1H-pyrrolo[3,2-c]pyridin-2-yl)sulfonyl)propanoate (0.70 g, 1.61 mmol, 56.2% yield) as a yellow solid. ¹H NMR (400 MHz, CDCl₃) δ 8.84 (s, 1H), 7.49 (s, 1H), 7.43 (s, 1H), 5.76 (s, 2H), 3.75 (t, J=7.5 Hz, 2H), 3.66-3.61 (m, 2H), 3.61 (s, 3H), 2.79 (t, J=7.4 Hz, 2H), 0.99-0.89 (m, 2H), 0.00 (s, 9H). MS (ESI): m/z 433.1 [M+1]⁺.

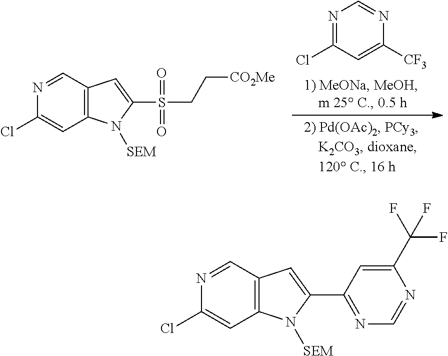 [SMILES: FC(F)(F)c1cc(Cl)ncn1 ; COC(=O)CCS(=O)(=O)c1cc2cnc(Cl)cc2n1COCC[Si](C)(C)C ; C[Si](C)(C)CCOCn1c(-c2cc(C(F)(F)F)ncn2)cc2cnc(Cl)cc21]

6-Chloro-2-(6-(trifluoromethyl)pyrimidin-4-yl)-1-((2-(trimethylsilyl)ethoxy)methyl)-1H-pyrrolo[3,2-c]pyridine. To a solution of methyl methyl 3-((6-chloro-1-((2-(trimethylsilyl)ethoxy)methyl)-1H-pyrrolo[3,2-c]pyridin-2-yl)sulfonyl)propanoate (500. mg, 1.15 mmol) in methanol (0.3333 mL) was added sodium methanolate (62.36 mg, 1.15 mmol), the mixture was stirred at 25° C. for 1 h. LCMS showed methyl 3-((6-chloro-1-((2-(trimethylsilyl)ethoxy)methyl)-1H-pyrrolo[3,2-c]pyridin-2-yl)sulfonyl)propanoate was consumed completely and intermediate formed. The mixture was concentrated under vacuum. The residue in 1, 4-dioxane (0.3333 mL) were added 4-chloro-6-(trifluoromethyl)pyrimidine (316.17 mg, 1.73 mmol), potassium carbonate (239.04 mg, 1.73 mmol), tricyclohexylphosphine (64.77 mg, 0.2300 mmol) and palladium(II) acetate (25.93 mg, 0.1200 mmol) under glove-box, the mixture was stirred at 120° C. for 16 h. LCMS showed intermediate was consumed completely and a peak (52%) with desired mass. The mixture was concentrated under vacuum. The residue was purified by flash silica gel chromatography (Biotage, 20 g SepaFlash® Silica Flash Column, eluent of 30˜60% ethyl acetate/petroleum ether gradient @ 30 mL/min) to give crude 6-chloro-2-(6-(trifluoromethyl)pyrimidin-4-yl)-1-((2-(trimethylsilyl)ethoxy)methyl)-1H-pyrrolo[3,2-c]pyridine (400 mg, 0.93 mmol, 80.7% yield) as a yellow oil. MS (ESI): m/z 429.1 [M+1]⁺.

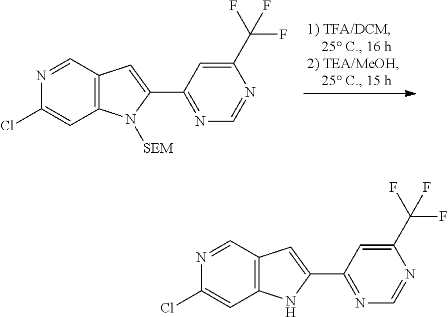 [SMILES: C[Si](C)(C)CCOCn1c(-c2cc(C(F)(F)F)ncn2)cc2cnc(Cl)cc21 ; FC(F)(F)c1cc(-c2cc3cnc(Cl)cc3[nH]2)ncn1]

6-Chloro-2-(6-(trifluoromethyl)pyrimidin-4-yl)-1H-pyrrolo[3,2-c]pyridine. To a solution of 6-chloro-2-(6-(trifluoromethyl)pyrimidin-4-yl)-1-((2-(trimethylsilyl)ethoxy)methyl)-1H-pyrrolo[3,2-c]pyridine (400. mg, 0.9300 mmol) in dichloromethane (2 mL) was added trifluoroacetic acid (7.55 mL, 99.31 mmol), the reaction mixture was stirred at 25° C. for 16 h. LCMS showed 26-chloro-2-(6-(trifluoromethyl)pyrimidin-4-yl)-1-((2-(trimethylsilyl)ethoxy)methyl)-1H-pyrrolo[3,2-c]pyridine was consumed completely. The mixture was concentrated under vacuum to give a residue. Dissolve the residue in methanol (2 mL), then triethylamine (7.55 mL, 54.16 mmol) was added to the solution and stirred at 25° C. for 2 h. LCMS showed a peak 88% desired mass. The mixture was concentrated under vacuum to give a residue. The residue was purified by prep-HPLC (column Phenomenex luna C18 150*25 mm*10 um; mobile phase: [water (0.225% FA)-ACN]; B %: 7%-37%, 10 min) followed by lyophilization to give 6-chloro-2-(6-(trifluoromethyl)pyrimidin-4-yl)-1H-pyrrolo[3,2-c]pyridine (200 mg, 0.66 mmol, 71.8% yield) as a yellow solid. ¹H NMR (400 MHz, DMSO-d₆) δ 12.80-12.36 (m, 1H), 9.47 (d, J=0.8 Hz, 1H), 8.81 (d, J=0.9 Hz, 1H), 8.68 (d, J=1.3 Hz, 1H), 7.92 (d, J=0.9 Hz, 1H), 7.48 (t, J=0.9 Hz, 1H).

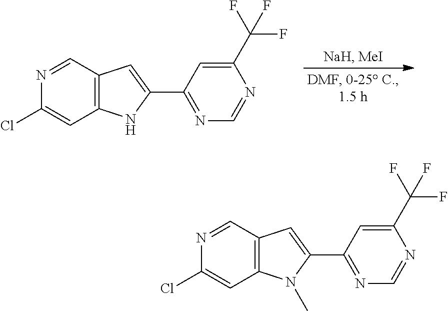

6-Chloro-1-methyl-2-(6-(trifluoromethyl)pyrimidin-4-yl)-1H-pyrrolo[3, 2-c]pyridine. To a solution of 6-chloro-2-(6-(trifluoromethyl)pyrimidin-4-yl)-1H-pyrrolo[3,2-c]pyridine (200. mg, 0.6700 mmol) in DMF (10 mL) was added sodium hydride (53.57 mg, 1.34 mmol) (60% purity) at 0° C., the mixture was stirred at 25° C. for 0.5 h. Then iodomethane (142.58 mg, 1 mmol) was added to the mixture at 0° C. and stirred at 25° C. for 1 h. TLC (petroleum ether:ethyl acetate=3:1, Rf SM=0.3, Rf DP=0.4) indicated starting material was consumed and one major new spot with lower polarity. The mixture was poured into saturated ammonium chloride aqueous (50 mL). The aqueous phase was extracted with ethyl acetate (30 mL×2). The combined organic phase was washed with brine (20 mL×2), dried over anhydrous sodium sulfate, filtered and concentrated under vacuum. The residue was purified by flash silica gel chromatography (Biotage, 12 g SepaFlash® Silica Flash Column, eluent of 30˜70% ethyl acetate/petroleum ether gradient @ 20 mL/min) to give 6-chloro-1-methyl-2-(6-(trifluoromethyl)pyrimidin-4-yl)-1H-pyrrolo[3, 2-c]pyridine (100 mg, 0.32 mmol, 47.7% yield) as a white solid. ¹H NMR (400 MHz, DMSO-d₆) δ 9.51 (s, 1H), 8.81 (d, J=0.8 Hz, 1H), 8.60 (d, J=1.0 Hz, 1H), 7.85 (s, 1H), 7.85 (s, 1H), 4.16 (s, 3H). MS (ESI): m/z 312.8 [M+1]⁺.

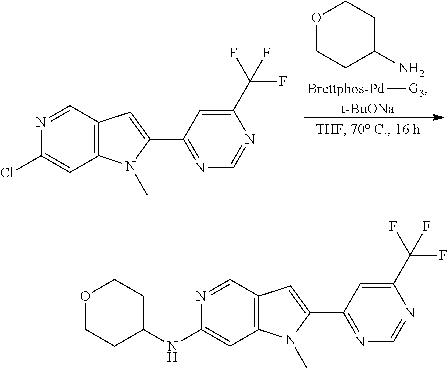

1-Methyl-N-(tetrahydro-2H-pyran-4-yl)-2-(6-(trifluoromethyl)pyrimidin-4-yl)-1H-pyrrolo[3, 2-c]pyridin-6-amine. To a solution of 6-chloro-1-methyl-2-(6-(trifluoromethyl)pyrimidin-4-yl)-1H-pyrrolo[3, 2-c]pyridine (100. mg, 0.3200 mmol) and tetrahydropyran-4-amine (97.05 mg, 0.9600 mmol) in THF (10 mL) were added methanesulfonato(2-dicyclohexylphosphino-3, 6-dimethoxy-2,4,6-tri-i-propyl-1,1-biphenyl)(2-amino-1, 1-biphenyl-2-yl)palladium(II) (28.99 mg, 0.0300 mmol) and sodium tert-butoxide (0.32 mL, 0.6400 mmol) (2 M in THF), the mixture was stirred at 70° C. for 16 h under nitrogen. LCMS showed 6-chloro-1-methyl-2-(6-(trifluoromethyl)pyrimidin-4-yl)-1H-pyrrolo[3, 2-c]pyridine was consumed completely and a peak (47%) with desired mass. The mixture was filtered with a pad of silica gel (100 mesh) and the filtrate was concentrated under vacuum. The residue was purified by prep-HPLC (column: Phenomenex Synergi C18 150*25 mm*10 um; mobile phase: [water(0.225% FA) -ACN];B %: 9%-39%, 10 min), then dried by lyophilization to give pure product and crude product. The crude product was purified by prep-HPLC (column: Unisil 3-100 C18 μLtra 150*50 mm*3 um; mobile phase: [water (0.225% FA) -ACN]; B %: 15%-35%, 10 min) again, then dried by lyophilization together to give 1-methyl-N-(tetrahydro-2H-pyran-4-yl)-2-(6-(trifluoromethyl)pyrimidin-4-yl)-1H-pyrrolo[3, 2-c]pyridin-6-amine (44.63 mg, 0.11 mmol, 35.6% yield, Formic acid) as a yellow solid. ¹H NMR (400 MHz, CD30D) δ 9.29 (d, J=0.7 Hz, 1H), 8.45-8.40 (m, 1.4H), 8.26 (d, J=1.2 Hz, 1H), 7.49 (d, J=0.7 Hz, 1H), 6.49 (s, 1H), 4.58 (s, 2H), 4.09 (s, 3H), 4.09-3.98 (m, 2H), 3.90-3.86 (m, 1H), 3.64-3.55 (m, 2H), 2.07-2.03 (m, 2H), 1.65-1.50 (m, 2H). MS (ESI): m/z 378.1 [M+1]⁺.

### Example 47: 2-(2-Isopropylpyrimidin-4-yl)-1-methyl-N-(tetrahydro-2H-pyran-4-yl)-1H-pyrrolo[3,2-c]pyridin-6-amine

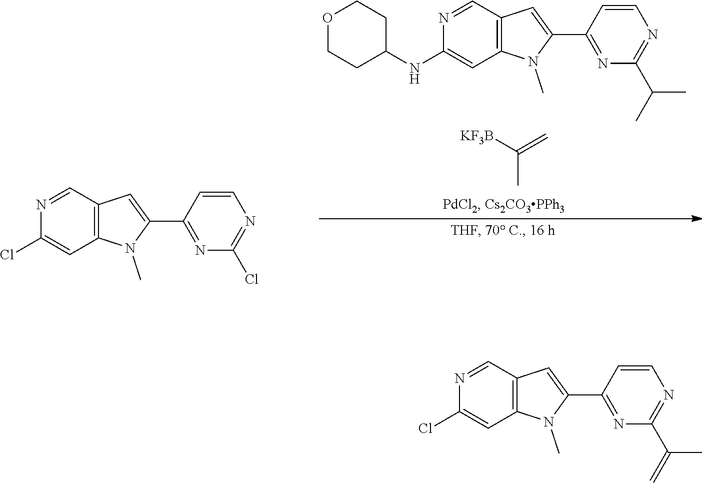

6-Chloro-1-methyl-2-(2-(prop-1-en-2-yl)pyrimidin-4-yl)-1H-pyrrolo[3,2-c]pyridine. To a mixture of 6-chloro-2-(2-chloropyrimidin-4-yl)-1-methyl-1H-pyrrolo[3,2-c]pyridine (400. mg, 1.43 mmol) in THF (2 mL) was added potassium hydride; trifluoro(isopropenyl)boron (256.2 mg, 1.72 mmol), caesium carbonate (933.85 mg, 2.87 mmol), palladium dichloride (25.41 mg, 0.1400 mmol) and triphenylphosphine (75.18 mg, 0.2900 mmol). The mixture was stirred at 70° C. for 16 h. LCMS showed 6-chloro-2-(2-chloropyrimidin-4-yl)-1-methyl-1H-pyrrolo[3,2-c]pyridine was consumed completely and a peak (52%) with desired mass. The mixture was cooled and concentrated in reduced pressure to give a residue. The residue was purified by flash silica gel chromatography (Biotage, 12 g SepaFlash® Silica Flash Column, Eluent of 30-50% Ethyl acetate/Petroleum ether gradient @35 mL/min to give 6-chloro-1-methyl-2-(2-(prop-1-en-2-yl)pyrimidin-4-yl)-1H-pyrrolo[3,2-c]pyridine (250 mg, 0.87 mmol, 61.2% yield) as a yellow solid. ¹H NMR (400 MHz, DMSO-d₆) δ 8.89 (d, J=5.3 Hz, 1H), 8.76 (d, J=0.8 Hz, 1H), 7.94 (d, J=5.3 Hz, 1H), 7.82 (s, 1H), 7.54 (d, J=0.8 Hz, 1H), 6.47 (d, J=1.4 Hz, 1H), 5.66-5.63 (m, 1H), 5.61-5.58 (m, 1H), 4.18 (s, 3H), 2.24 (s, 3H). MS (ESI): m/z 285.1 [M+1]⁺.

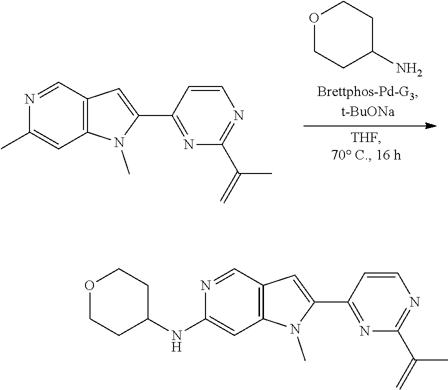 [SMILES: NC1CCOCC1 ; C=C(C)c1nccc(-c2cc3cnc(C)cc3n2C)n1 ; C=C(C)c1nccc(-c2cc3cnc(NC4CCOCC4)cc3n2C)n1]

1-Methyl-2-(2-(prop-1-en-2-yl)pyrimidin-4-yl)-N-(tetrahydro-2H-pyran-4-yl)-1H-pyrrolo[3,2-c]pyridin-6-amine. To a solution of 6-chloro-1-methyl-2-(2-(prop-1-en-2-yl)pyrimidin-4-yl)-1H-pyrrolo[3,2-c]pyridine (266.43 mg, 2.63 mmol) in THF (10 mL) were added methanesulfonato(2-dicyclohexylphosphino-3,6-dimethoxy-2,4,6-tri-i-propyl-1,1-biphenyl)(2-amino-1,1-biphenyl-2-yl)palladium(II) (79.59 mg, 0.0900 mmol) and sodium tert-butoxide (0.88 mL, 1.76 mmol) (2 M in THF), the mixture was stirred at 70° C. for 16 h under nitrogen. LCMS showed 6-chloro-2-(2-isopropenylpyrimidin-4-yl)-1-methyl-pyrrolo[3,2-c]pyridine was consumed completely and a peak (68%) with desired mass. The mixture was concentrated under vacuum. The residue was purified by flash silica gel chromatography (Biotage, 4 g SepaFlash® Silica Flash Column, Eluent of 30˜100% Ethyl acetate/Petroleum ether gradient @ 60 mL/min, TLC (petroleum ether:ethyl acetate=1:1, Rf DP=0.2) to give 1-methyl-2-(2-(prop-1-en-2-yl)pyrimidin-4-yl)-N-(tetrahydro-2H-pyran-4-yl)-1H-pyrrolo[3,2-c]pyridin-6-amine (280 mg, 0.72 mmol, 81.9% yield) as a yellow oil. MS (ESI): m/z 350.2 [M+1]⁺.

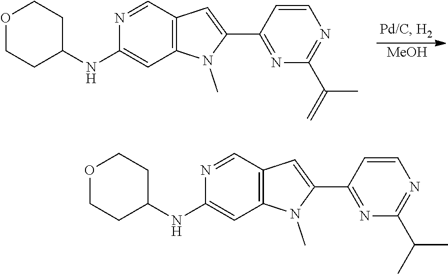

2-(2-isopropylpyrimidin-4-yl)-1-methyl-N-(tetrahydro-2H-pyran-4-yl)-1H-pyrrolo[3,2-c]pyridin-6-amine. To a solution of 1-methyl-2-(2-(prop-1-en-2-yl)pyrimidin-4-yl)-N-(tetrahydro-2H-pyran-4-yl)-1H-pyrrolo[3,2-c]pyridin-6-amine (104.18 mg, 0.3000 mmol) in methanol (10 mL) was added Pd/C (31.73 mg, 0.3000 mmol) under nitrogen, the mixture was stirred at 25° C. for 16 h under molecular hydrogen (0.6 mg, 0.3000 mmol) (15 Psi). LCMS showed 1-methyl-2-(2-(prop-1-en-2-yl)pyrimidin-4-yl)-N-(tetrahydro-2H-pyran-4-yl)-1H-pyrrolo[3,2-c]pyridin-6-amine was consumed completely and a peak with desired mass. The mixture was filtered with a pad of silica gel (100-200 mesh) and the filtrate was concentrated under vacuum. The residue was purified by prep-HPLC (column: Unisil 3-100 C18 μLtra 150*50 mm*3 um; mobile phase: [water(0.225% FA) -ACN]; B %: 18%-38%, 10 min), then dried by lyophilization to give 2-(2-isopropylpyrimidin-4-yl)-1-methyl-N-(tetrahydro-2H-pyran-4-yl)-1H-pyrrolo[3,2-c]pyridin-6-amine (114.86 mg, 0.32 mmol, 100% yield) as a yellow solid. ¹H NMR (400 MHz, methanol-d₄) δ 8.61 (d, J=5.5 Hz, 1H), 8.39 (d, J=0.6 Hz, 1H), 7.69 (d, J=5.5 Hz, 1H), 7.23 (s, 1H), 6.49 (s, 1H), 4.09 (s, 3H), 4.02-3.97 (m, 2H), 3.91-3.81 (m, 1H), 3.63-3.56 (m, 2H), 3.23-3.20 (m, 1H), 2.07-2.02 (m, 2H), 1.63-1.50 (m, 2H), 1.40 (d, J=6.8 Hz, 6H). MS (ESI): m/z 352.2 [M+1]⁺.

### Example 48: N,1-Dimethyl-2-[2-(2,2,2-trifluoroethylamino)pyrimidin-4-yl]pyrrolo[3,2-c]pyridin-6-amine

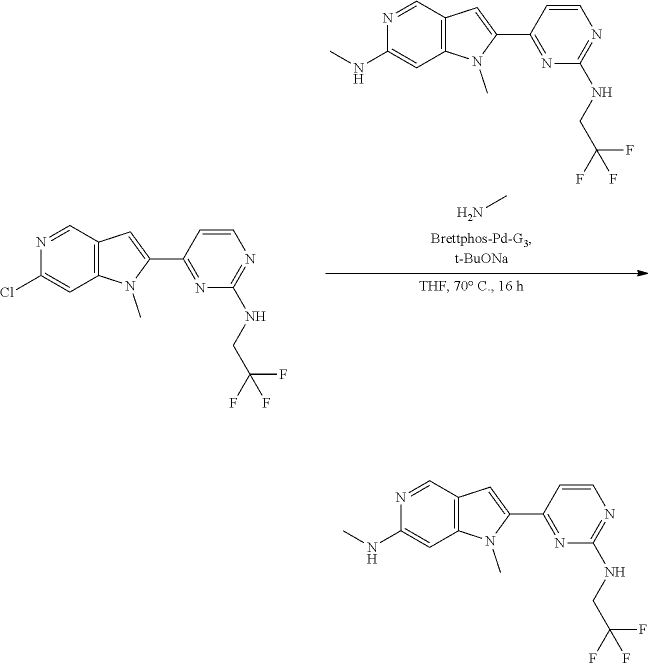

N,1-dimethyl-2-[2-(2,2,2-trifluoroethylamino)pyrimidin-4-yl]pyrrolo[3,2-c]pyridin-6-amine. To a mixture of 4-(6-chloro-1-methyl-pyrrolo[3,2-c]pyridin-2-yl)-N-(2,2,2-trifluoroethyl)pyrimidin-2-amine (300 mg, 0.88 mmol) in THF (5 mL) was added methanamine (1.32 mL, 2.63 mmol), sodium tert-butoxide (0.88 mL, 1.76 mmol, 2 M in THF) and methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II) (159.49 mg, 0.18 mmol). The mixture was stirred at 70° C. for 16 h under nitrogen. LCMS showed a peak (46%) with desired mass. The reaction mixture was filtered and the filtrate was concentrated. The residue obtained was purified by prep-HPLC (column: Phenomenex Luna C18 150*40 mm*15 um; mobile phase: [water (0.225% FA)-ACN]; B %: 12%-42%, 10 min) followed by lyophilization to give N,1-dimethyl-2-[2-(2,2,2-trifluoroethylamino)pyrimidin-4-yl]pyrrolo[3,2-c]pyridin-6-amine (111.5 mg, 0.33 mmol, 37.4% yield, formic acid) as a yellow solid. ¹H NMR (400 MHz, DMSO-d₆) δ 8.40 (d, J=0.6 Hz, 1H), 8.32 (d, J=5.4 Hz, 1H), 8.22 (s, 0.6H), 7.80 (t, J=6.6 Hz, 1H), 7.20-7.15 (m, 2H), 6.27 (s, 1H), 6.14 (d, J=4.4 Hz, 1H), 4.23-4.13 (m, 2H), 3.99 (s, 3H), 2.80 (d, J=3.8 Hz, 3H); MS (ESI): m/z 337.2 [M+1]⁺.

### Example 49: 2-(6-Ethylpyrimidin-4-yl)-1-methyl-N-tetrahydropyran-4-yl-pyrrolo[3,2-c]pyridin-6-amine

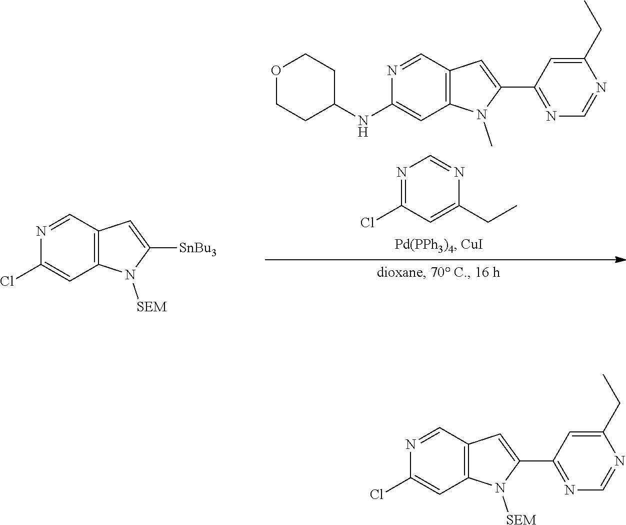

2-[[6-Chloro-2-(6-ethylpyrimidin-4-yl)pyrrolo[3,2-c]pyridin-1-yl]methoxy]ethyl-trimethyl-silane. To 2-[(6-chloro-2-tributylstannyl-pyrrolo[3,2-c]pyridin-1-yl)methoxy]ethyl-trimethyl-silane (2. g, 3.5 mmol) in 1,4-Dioxane (20 mL) was added 4-chloro-6-ethyl-pyrimidine (0.75 g, 5.25 mmol), copper(I)iodide (66.60 mg, 0.35 mmol) and tetrakis[triphenylphosphine]palladium(0) (404.12 mg, 0.35 mmol). The mixture was stirred at 70° C. for 16 h under nitrogen. LCMS showed a peak (53%) with desired mass. The reaction mixture was filtered and the filtrate was concentrated. The residue obtained was purified by flash silica gel chromatography (40 g SepaFlash® Silica Flash Column, Eluent of 20˜30% Ethyl acetate/Petroleum ether gradient@ 35 mL/min) to give 2-[[6-chloro-2-(6-ethylpyrimidin-4-yl)pyrrolo[3,2-c]pyridin-1-yl]methoxy]ethyl-trimethyl-silane (0.90 g, 2.23 mmol, 63.7% yield) as a yellow solid. MS (ESI): m/z 389.1 [M+1]⁺.

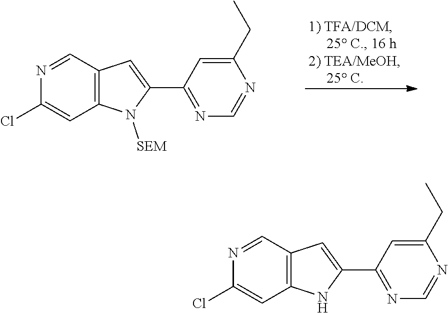 [SMILES: CCc1cc(-c2cc3cnc(Cl)cc3n2COCC[Si](C)(C)C)ncn1 ; CCc1cc(-c2cc3cnc(Cl)cc3[nH]2)ncn1]

6-Chloro-2-(6-ethylpyrimidin-4-yl)-1H-pyrrolo[3,2-c]pyridine. To a mixture of 2-[[6-chloro-2-(6-ethylpyrimidin-4-yl)pyrrolo[3,2-c]pyridin-1-yl]methoxy]ethyl-trimethyl-silane (0.9 g, 2.31 mmol) in dichloromethane (10 mL) was added trifluoroacetic acid (10 mL, 129.8 mmol). The mixture was stirred at 25° C. for 16 h under nitrogen. LCMS showed a peak (94%) with desired mass. The reaction mixture was concentrated under vacuum. The residue was added methanol (10 mL). The mixture was adjusted to pH=9 with triethylamine. The reaction mixture was concentrated under reduced pressure and purified by flash silica gel chromatography (20 g SepaFlash® Silica Flash Column, Eluent of 30˜40% Ethyl acetate/Petroleum ether gradient) to give 6-chloro-2-(6-ethylpyrimidin-4-yl)-1H-pyrrolo[3, 2-c]pyridine (500 mg, 1.82 mmol, 78.8% yield) as a yellow solid. MS (ESI): m/z 259.1 [M+1]⁺.

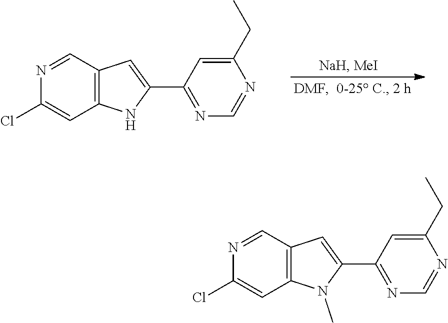 [SMILES: CCc1cc(-c2cc3cnc(Cl)cc3[nH]2)ncn1 ; CCc1cc(-c2cc3cnc(Cl)cc3n2C)ncn1]

6-Chloro-2-(6-ethylpyrimidin-4-yl)-1-methyl-pyrrolo[3,2-c]pyridine. To a mixture of 6-chloro-2-(6-ethylpyrimidin-4-yl)-1H-pyrrolo[3, 2-c]pyridine (0.5 g, 1.93 mmol) in DMF (60 mL) was added sodium hydride (0.12 g, 2.9 mmol, 60% purity) at 0° C. The mixture was stirred at 25° C. for 0.5 h under nitrogen. iodomethane (0.24 mL, 3.87 mmol) was added at 0° C. The mixture was stirred at 25° C. for 1.5 h under nitrogen. LCMS showed a peak (92%) with desired mass. The mixture was poured into cold saturated ammonium chloride (200 mL). The aqueous phase was extracted with ethyl acetate (100 mL×2). The combined organic phase was washed with brine (50 mL×4), dried over anhydrous sodium sulfate, filtered and concentrated under vacuum. The residue was purified by flash silica gel chromatography (20 g SepaFlash® Silica Flash Column, Eluent of 60-80% Ethyl acetate/Petroleum ether gradient) to give 6-chloro-2-(6-ethylpyrimidin-4-yl)-1-methyl-pyrrolo[3, 2-c]pyridine (400 mg, 1.47 mmol, 75.8% yield) as a yellow solid. MS (ESI): m/z 273.1 [M+1]⁺.

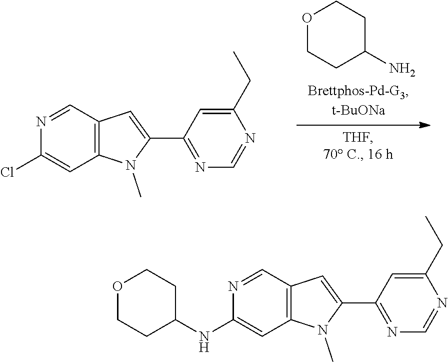 [SMILES: CCc1cc(-c2cc3cnc(Cl)cc3n2C)ncn1 ; NC1CCOCC1 ; CCc1cc(-c2cc3cnc(NC4CCOCC4)cc3n2C)ncn1]

2-(6-Ethylpyrimidin-4-yl)-1-methyl-N-tetrahydropyran-4-yl-pyrrolo[3,2-c]pyridin-6-amine. To a mixture of 6-chloro-2-(6-ethylpyrimidin-4-yl)-1-methyl-pyrrolo[3, 2-c]pyridine (400 mg, 1.47 mmol) and tetrahydropyran-4-amine (445.06 mg, 4.4 mmol) in tetrahydrofuran (5 mL) were added sodium tert-butoxide (1.47 mL, 2.93 mmol, 2 M in THF) and Methanesulfonato(2-dicyclohexylphosphino-3, 6-dimethoxy-2′,4′,6′-tri-i-propyl-1, 1′-biphenyl)(2′-amino-1, 1′-biphenyl-2-yl)palladium(II) (266.45 mg, 0.29 mmol). The mixture was stirred at 70° C. for 16 h under nitrogen. LCMS showed a peak (56%) with desired mass. The reaction mixture was concentrated under vacuum. The residue was purified by prep-HPLC (column: Phenomenex Luna Cis 150*40 mm*15 um; mobile phase: [water (0.225% FA)-ACN]; B %: 10%-40%, 10 min), followed by lyophilization to give 2-(6-ethylpyrimidin-4-yl)-1-methyl-N-tetrahydropyran-4-yl-pyrrolo[3, 2-c]pyridin-6-amine (256.23 mg, 0.76 mmol, 51.7% yield) as a yellow oil. ¹H NMR (400 MHz, DMSO-d₆) δ 9.06 (d, J=1.1 Hz, 1H), 8.42 (s, 1H), 7.81 (s, 1H), 7.25 (s, 1H), 6.38 (s, 1H), 6.12 (d, J=7.9 Hz, 1H), 3.96 (s, 3H), 3.91-3.84 (m, 3H), 3.46-3.39 (m, 2H), 2.76 (q, J=7.6 Hz, 2H), 1.93-1.88 (m, 2H), 1.50-1.39 (m, 2H), 1.27 (J=7.5 Hz, 3H); MS (ESI): m/z 338.3 [M+1]⁺.

### Example 50: 2-(6-Methoxypyrimidin-4-yl)-1-methyl-N-(tetrahydro-2H-pyran-4-yl)-1H-pyrrolo[3,2-c]pyridin-6-amine

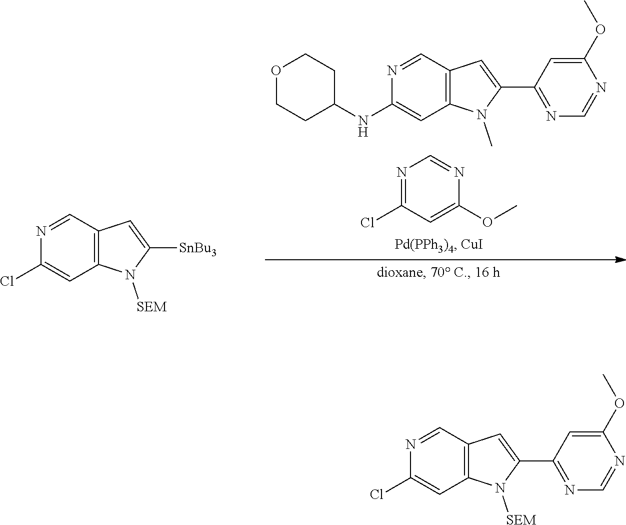

6-Chloro-2-(6-methoxypyrimidin-4-yl)-1-((2-(trimethylsilyl)ethoxy)methyl)-1H-pyrrolo[3,2-c]pyridine. To a solution of 2-[(6-chloro-2-tributylstannyl-pyrrolo[3,2-c]pyridin-1-yl)methoxy]ethyl-trimethyl-silane (1.5 g, 2.62 mmol) and 4-chloro-6-methoxy-pyrimidine (568.75 mg, 3.93 mmol) in 1,4-Dioxane (3 mL) were added tetrakis[triphenylphosphine]palladium(0) (303.09 mg, 0.2600 mmol) and copper iodide (49.95 mg, 0.26 mmol) under nitrogen, the mixture was stirred at 70° C. for 16 h. LCMS showed 2-[(6-chloro-2-tributylstannyl-pyrrolo[3,2-c]pyridin-1-yl)methoxy]ethyl-trimethyl-silane was consumed completely and a peak (30%) with desired mass. The mixture was concentrated under vacuum. The residue was purified by flash silica gel chromatography (Biotage, 20 g SepaFlash® Silica Flash Column, Eluent of 10˜20% Ethyl acetate/Petroleum ether gradient @ 35 mL/min) to give 2-[[6-chloro-2-(6-methoxypyrimidin-4-yl)pyrrolo[3,2-c]pyridin-1-yl]methoxy]ethyl-trimethyl-silane (730 mg, 1.45 mmol, 55.4% yield) as a brown oil. ¹H NMR (400 MHz, DMSO-d₆) δ 8.91 (d, J=1.1 Hz, 1H), 8.76 (d, J=0.6 Hz, 1H), 7.90 (s, 1H), 7.49 (d, J=1.1 Hz, 1H), 7.47 (d, J=0.6 Hz, 1H), 6.12 (s, 2H), 3.99 (s, 3H), 3.34 (t, J=7.8 Hz, 2H), 0.68 (t, J=7.8 Hz, 2H), −0.24 (s, 9H); MS (ESI): m/z 391.2 [M+1]⁺.

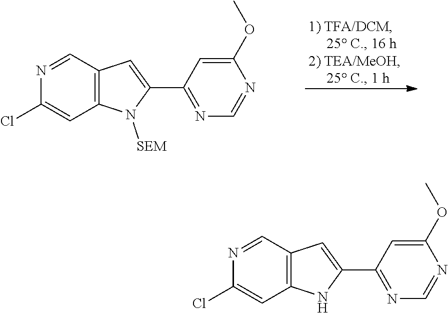 [SMILES: COc1cc(-c2cc3cnc(Cl)cc3n2COCC[Si](C)(C)C)ncn1 ; COc1cc(-c2cc3cnc(Cl)cc3[nH]2)ncn1]

6-Chloro-2-(6-methoxypyrimidin-4-yl)-1H-pyrrolo[3,2-c]pyridine. To a solution of 2-[[6-chloro-2-(6-methoxypyrimidin-4-yl)pyrrolo[3,2-c]pyridin-1-yl]methoxy]ethyl-trimethyl-silane (730 mg, 1.87 mmol) in dichloromethane (10 mL) was added Trifluoroacetic acid (10 mL, 1.87 mmol), the mixture was stirred at 25° C. for 16 h. LCMS showed 2-[[6-chloro-2-(6-methoxypyrimidin-4-yl)pyrrolo[3,2-c]pyridin-1-yl]methoxy]ethyl-trimethyl-silane was consumed completely and a peak (45%) with desired mass. The mixture was concentrated under oil pump. The residue in methanol (10 mL) was added triethylamine (10 mL, 1.87 mmol), the mixture was stirred at 25° C. for 1 h. LCMS showed a main peak with desired mass. The mixture was concentrated under vacuum. The residue was triturated with methanol (10 mL) for 0.5 h. The suspension was filtered and the filtered cake was dried under vacuum to give 6-chloro-2-(6-methoxypyrimidin-4-yl)-1H-pyrrolo[3,2-c]pyridine (330 mg, 1.26 mmol, 67.7% yield) as a grey solid. ¹H NMR (400 MHz, DMSO-d₆) δ 12.39 (s, 1H), 8.88 (s, 1H), 8.75-8.72 (m, 1H), 7.59 (s, 1H), 7.45 (s, 1H), 7.33-7.28 (m, 1H), 3.99 (s, 3H); MS (ESI): m/z 261.1 [M+1]⁺.

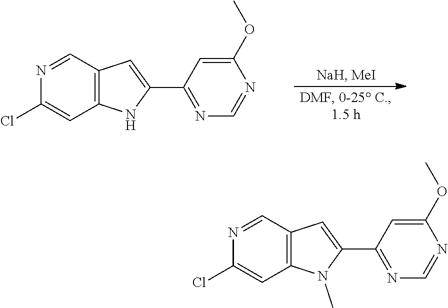 [SMILES: COc1cc(-c2cc3cnc(Cl)cc3[nH]2)ncn1 ; COc1cc(-c2cc3cnc(Cl)cc3n2C)ncn1]

6-Chloro-2-(6-methoxypyrimidin-4-yl)-1-methyl-1H-pyrrolo[3,2-c]pyridine. To a solution of 6-chloro-2-(6-methoxypyrimidin-4-yl)-1H-pyrrolo[3,2-c]pyridine (330. mg, 1.27 mmol) in DMF (10 mL) was added sodium hydride (101.27 mg, 2.53 mmol, 60% purity) at 0° C., the mixture was stirred at 25° C. for 0.5 h. Then iodomethane (269.53 mg, 1.9 mmol) was added to the mixture at 0° C. and stirred at 25° C. for 1 h. TLC (Petroleum ether:Ethyl acetate=3:1, Rf SM=0.5, Rf DP=0.6) indicated starting material remained and one major new spot with lower polarity. LCMS showed 6-chloro-2-(6-methoxypyrimidin-4-yl)-1H-pyrrolo[3,2-c]pyridine remained and a peak (66%) with desired mass. The mixture was poured into saturated ammonium chloride aqueous (100 mL). The aqueous phase was extracted with ethyl acetate (50 mL×2). The combined organic phase was washed with brine (20 mL×3), dried over anhydrous sodium sulfate, filtered and concentrated under vacuum. The residue was purified by flash silica gel chromatography (Biotage, 12 g SepaFlash® Silica Flash Column, Eluent of 30˜60% Ethyl acetate/Petroleum ether gradient @ 30 mL/min) to give 6-chloro-2-(6-methoxypyrimidin-4-yl)-1-methyl-pyrrolo[3,2-c]pyridine (160 mg, 0.58 mmol, 46.0% yield) as a white solid. ¹H NMR (400 MHz, DMSO-d₆) δ 8.92 (d, J=1.0 Hz, 1H), 8.73 (d, J=0.6 Hz, 1H), 7.47 (d, J=1.0 Hz, 1H), 7.39 (d, J=0.8 Hz, 1H), 4.07 (s, 3H), 4.00 (s, 3H); MS (ESI): m/z 275.1 [M+1]⁺.

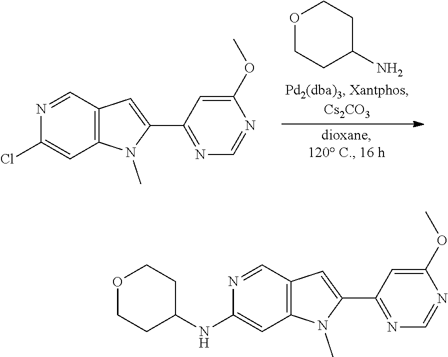 [SMILES: COc1cc(-c2cc3cnc(Cl)cc3n2C)ncn1 ; NC1CCOCC1 ; COc1cc(-c2cc3cnc(NC4CCOCC4)cc3n2C)ncn1]

2-(6-Methoxypyrimidin-4-yl)-1-methyl-N-tetrahydropyran-4-yl-pyrrolo[3,2-c]pyridin-6-amine. To a solution of 6-chloro-2-(6-methoxypyrimidin-4-yl)-1-methyl-pyrrolo[3,2-c]pyridine (150 mg, 0.55 mmol) and tetrahydropyran-4-amine (165 mg, 1.64 mmol) in 1,4-Dioxane (5 mL) were added tris(dibenzylideneacetone)dipalladium(0) (50 mg, 0.050 mmol), (5-diphenylphosphinyl-9,9-dimethylxanthen-4-yl)-diphenylphosphine (31.59 mg, 0.0500 mmol) and cesium carbonate (533.72 mg, 1.64 mmol), the mixture was stirred at 70° C. for 16 h. LCMS showed (5-diphenylphosphinyl-9,9-dimethylxanthen-4-yl)-diphenylphosphine remained but no desired mass. All the reagents was added as above to the mixture and stirred at 100° C. for 6 h. LCMS showed (5-diphenylphosphinyl-9,9-dimethylxanthen-4-yl)-diphenylphosphine remained (37%) and a peak (4%) with desired mass. All the reagents was added to the mixture as above and stirred at 120° C. for 16 h. LCMS showed (5-diphenylphosphinyl-9,9-dimethylxanthen-4-yl)-diphenylphosphine was consumed and a peak (45%) with desired mass was detected. The mixture was filtered and the filtrate was concentrated under vacuum. The residue obtained was purified by flash silica gel chromatography (Biotage, 4 g SepaFlash® Silica Flash Column, Eluent of 30˜100% Ethyl acetate/Petroleum ether gradient @ 20 mL/min, TLC (Petroleum ether:Ethyl acetate=0:1, Rf=0.6) to give a crude product. The crude product was purified by prep-HPLC (column: Unisil 3-100 C18 Ultra 150*50 mm*3 um; mobile phase: [water (0.225% FA)-ACN]; B %: 15%-35%, 10 min), then dried by lyophilization to give 2-(6-methoxypyrimidin-4-yl)-1-methyl-N-tetrahydropyran-4-yl-pyrrolo[3,2-c]pyridin-6-amine (14.17 mg, 0.041 mmol, 7.51% yield, formic acid) as a yellow solid. ¹H NMR (400 MHz, CD3OD) δ 8.82 (d, J=1.0 Hz, 1H), 8.40-8.36 (m, 2H), 7.25 (d, J=1.1 Hz, 1H), 7.12 (d, J=0.7 Hz, 1H), 6.68 (s, 1H), 4.05 (s, 3H), 4.02-3.97 (m, 2H), 3.97 (s, 3H), 3.89-3.82 (m, 1H), 3.63-3.57 (m, 2H), 2.07-2.04 (m, 2H), 1.63-1.56 (m, 2H); MS (ESI): m/z 340.2 [M+1]⁺.

### Example 51: 2-(2-Ethylpyrimidin-4-yl)-1-methyl-N-(tetrahydro-2H-pyran-4-yl)-1H-pyrrolo[3,2-c]pyridin-6-amine

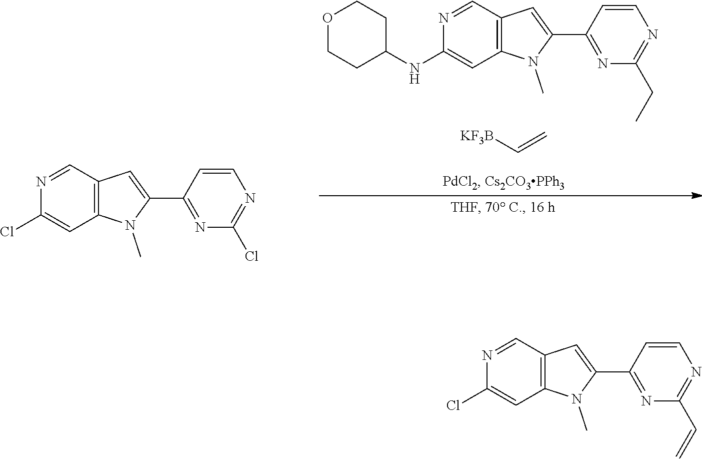

6-Chloro-1-methyl-2-(2-vinylpyrimidin-4-yl)-1H-pyrrolo[3,2-c]pyridine. To 6-chloro-2-(2-chloropyrimidin-4-yl)-1-methyl-1H-pyrrolo[3,2-c]pyridine (300 mg, 1.07 mmol) in THF (2 mL) was added potassium hydride; trifluoro(vinyl)boron (174 mg, 1.29 mmol), caesium carbonate (700 mg, 2.15 mmol), palladium dichloride (19 mg, 0.11 mmol) and triphenylphosphine (56 mg, 0.21 mmol). The mixture was stirred at 70° C. for 16 h. LCMS showed 6-chloro-2-(2-chloropyrimidin-4-yl)-1-methyl-1H-pyrrolo[3,2-c]pyridine remained and a peak with desired mass. The mixture was concentrated in reduced pressure to give a residue, which was purified by flash silica gel chromatography (Biotage, 12 g SepaFlash® Silica Flash Column, Eluent of 30˜60% Ethyl acetate/Petroleum ether gradient @ 35 mL/min) to give a crude product. The crude product was reacted again with the same reagents as before. LCMS showed 6-chloro-2-(2-chloropyrimidin-4-yl)-1-methyl-1H-pyrrolo[3,2-c]pyridine was consumed completely and a peak (52%) with desired mass. The mixture was cooled and concentrated under reduced pressure to give a residue, which was purified by flash silica gel chromatography (Biotage, 12 g SepaFlash® Silica Flash Column, eluent of 30˜60% ethyl acetate/petroleum ether gradient @ 35 mL/min) to give 6-chloro-1-methyl-2-(2-vinylpyrimidin-4-yl)-1H-pyrrolo[3,2-c]pyridine (200 mg, 0.58 mmol, 54.8% yield) as a yellow solid. ¹H NMR (400 MHz, DMSO-d₆) δ 8.87 (d, J=5.3 Hz, 1H), 8.76 (d, J=0.8 Hz, 1H), 7.92 (d, J=5.3 Hz, 1H), 7.82 (s, 1H), 7.52 (d, J=0.8 Hz, 1H), 6.94-6.87 (m, 1H), 6.65-6.60 (m, 1H), 5.89-5.77 (m, 1H), 4.17 (s, 3H); MS (ESI): m/z 270.8 [M+1]⁺.

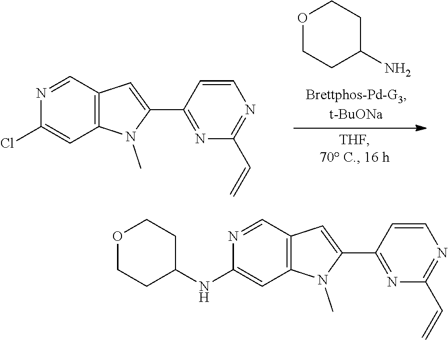

1-Methyl-N-(tetrahydro-2H-pyran-4-yl)-2-(2-vinylpyrimidin-4-yl)-1H-pyrrolo[3,2-c]pyridin-6-amine. To a solution of 6-chloro-1-methyl-2-(2-vinylpyrimidin-4-yl)-1H-pyrrolo[3,2-c]pyridine (200 mg, 0.74 mmol) and tetrahydropyran-4-amine (224.18 mg, 2.22 mmol) in THF (10 mL) were added methanesulfonato(2-dicyclohexylphosphino-3,6-dimethoxy-2,4,6-tri-i-propyl-1,1-biphenyl)(2-amino-1, 1-biphenyl-2-yl)palladium(II) (67 mg, 0.070 mmol) and sodium tert-butoxide (0.74 mL, 1.48 mmol) (2 M in THF). The mixture was stirred at 70° C. for 16 h under nitrogen. LCMS showed 6-chloro-1-methyl-2-(2-vinylpyrimidin-4-yl)-1H-pyrrolo[3,2-c]pyridine was consumed completely and a peak (42%) with desired mass. The mixture was concentrated and purified by flash silica gel chromatography (Biotage, 12 g SepaFlash® Silica Flash Column, eluent of 30˜100% ethyl acetate/petroleum ether gradient @20 mL/min) to give 1-methyl-N-(tetrahydro-2H-pyran-4-yl)-2-(2-vinylpyrimidin-4-yl)-1H-pyrrolo[3,2-c]pyridin-6-amine (100 mg, 0.29 mmol, 40.3% yield) as a white solid. MS (ESI): m/z 336.2 [M+1]⁺.

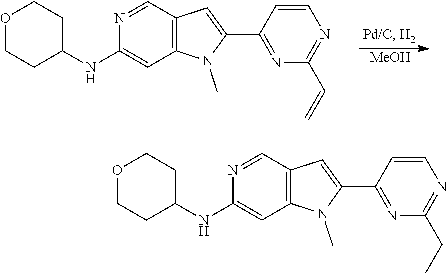

2-(2-Ethylpyrimidin-4-yl)-1-methyl-N-(tetrahydro-2H-pyran-4-yl)-1H-pyrrolo[3,2-c]pyridin-6-amine. To a solution of 1-methyl-N-(tetrahydro-2H-pyran-4-yl)-2-(2-vinylpyrimidin-4-yl)-1H-pyrrolo[3,2-c]pyridin-6-amine (100 mg, 0.30 mmol) in methanol (10 mL) was added Pd/C (31 mg, 0.30 mmol) under nitrogen, the mixture was stirred at 25° C. for 16 h under molecular hydrogen (15 Psi). LCMS showed 1-methyl-N-tetrahydropyran-4-yl-2-(2-vinylpyrimidin-4-yl)pyrrolo[3,2-c]pyridin-6-amine was consumed completely and a peak with desired mass. The mixture was filtered with a pad of silica gel (100-200 mesh), the filtrate was concentrated under vacuum. The residue obtained was purified by prep-HPLC (column: Phenomenex luna Cis 150*25 mm*10 um; mobile phase: [water (0.225% FA)-ACN]; B %: 7%-37%, 10 min), then dried by lyophilization to give 2-(2-ethylpyrimidin-4-yl)-1-methyl-N-(tetrahydro-2H-pyran-4-yl)-1H-pyrrolo[3,2-c]pyridin-6-amine (12.92 mg, 0.038 mmol, 12.8% yield) as a yellow solid. ¹H NMR (400 MHz, METHANOL-d₄) δ 8.64 (d, J=5.5 Hz, 1H), 8.43-8.36 (m, 1.5H), 7.71 (d, J=5.5 Hz, 1H), 7.26 (s, 1H), 6.57 (s, 1H), 4.09 (s, 3H), 4.02-3.98 (m, 2H), 3.93-3.81 (m, 1H), 3.63-3.57 (m, 2H), 3.01 (q, J=7.6 Hz, 2H), 2.07-2.03 (m, 2H), 1.66-1.50 (m, 2H), 1.42 (t, J=7.6 Hz, 3H); MS (ESI): m/z 338.3 [M+1]⁺.

Activity of certain Heterocyclic Compounds in the C. parvum growth inhibition assays is shown in Table 1.

C. parvum GROWTH INHIBITION ASSAYS

C. parvum growth inhibition was performed as described in Hulverson M A, Vinayak S, Choi R, Schaefer D A, Castellanos-Gonzalez A, Vidadala R S R, Brooks C F, Herbert G T, Betzer D P, Whitman G R, Sparks H N, Arnold S L M, Rivas K L, Barrett L K, White A C Jr, Maly D J, Riggs M W, Striepen B, Van Voorhis W C, Ojo K K. Bumped-Kinase Inhibitors for Cryptosporidiosis Therapy. J Infect Dis. 2017 Apr. 15; 215(8):1275-1284.

C. parvum strain UGA1 adenocarcinoma (HCT-8) cells expressing human ileocecal Nluc in (ATCC, Manassas, VA) were prepared as described below. HCT-8 cells were inoculated onto 96-well plates were grown for 48 hours to 90%-100% confluence. C. parvum oocysts were then incubated for 10 minutes in 10% bleach at room temperature and washed with Dulbecco's phosphate-buffered saline. One thousand oocysts per well were applied to plates with Roswell Park Memorial Institute 1640 (RPMI) medium supplemented with 10% horse serum and 1% penicillin/streptomycin. Compounds of Formula (I), Formula (II), and Formula (III) were added to the plates at different concentrations and the plates were incubated for 48 hours. Following the incubation, the monolayers were lysed for 1 hour before a luciferase reagent was added, and relative luminescence units were determined.

For certain compounds of Formula I, Formula II, and Formula III, EC50 values were calculated using curves calculated from Prism after analysis of C. parvum growth inhibition at 6 different concentrations: 10, 3.33, 1.11, 0.37, 0.12, and 0.04 μM. In Table 1 below, certain compounds of Formula I, Formula II, and Formula III had an EC₅₀ greater than 1 M (activity level A), an EC₅₀ between 0.5 μM and 1 μM (activity level B), and an EC₅₀ below 0.5 μM (activity level C). Additionally, for certain compounds of Formula I, Formula II, and Formula III, single point inhibition values were calculated at either 0.1 μM or 10 μM or both 0.1 M and 10 μM. For certain compounds of Formula I, Formula II, and Formula III, single point inhibition values of 0-33% (activity level D), between 33% and 66% (activity level E), and between 67% and 100% (activity level F) are also shown in Table 1 below.

TABLE 1

| | | | | | % inh @ | % inh @ |
|---|---|---|---|---|---|---|
| | | | | EC50 | 1 uM | 10 uM |
| Com. No. | STRUCTURE | Chemical Name | M + 1 | (uM) C. parvum | C. parvum | C. parvum |
| 1 | 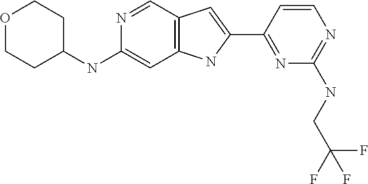 | N-(tetrahydro-2H-pyran-4-yl)-2- (2-(2,2,2- trifluoroethylamino)pyrimidin-4-yl)- 1H-pyrrolo[3,2-c]pyridin-6-amine | 393.3 | C | F | F |
| 2 | 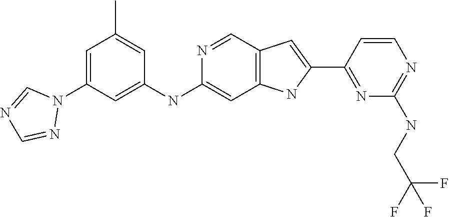 [SMILES: Cc1cc(Nc2cc3[nH]c(-c4ccnc(NCC(F)(F)F)n4)cc3cn2)cc(-n2cncn2)c1] | N-(3-methyl-5-(1H-1,2,4-triazol-1- yl)phenyl)-2-(2-(2,2,2- trifluoroethylamino)pyrimidin-4-yl)- 1H-pyrrolo[3,2-c]pyridin-6-amine | 466.2 | C | E | D |
| 3 | 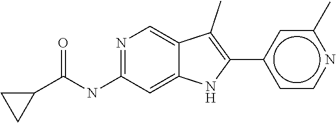 | N-(3-methyl-2-(2-methylpyridin-4- yl)-1H-pyrrolo[3,2-c]pyridin-6- yl)cyclopropanecarboxamide | 307.1 | | | |
| 4 | 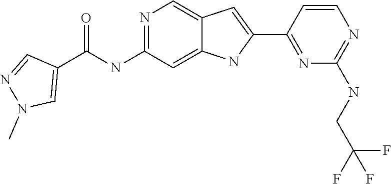 | 1-methyl-N-(2-(2-(2,2,2- trifluoroethylamino)pyrimidin-4-yl)- 1H-pyrrolo[3,2-c]pyridin-6-yl)-1H- pyrazole-4-carboxamide | 417.0 | C | F | F |
| 5 | 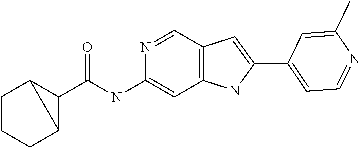 [SMILES: Cc1cc(-c2cc3cnc(NC(=O)C4C5CCCC54)cc3[nH]2)ccn1] | N-(2-(2-methylpyridin-4-yl)-1H- pyrrolo[3,2-c]pyridin-6- yl)bicyclo[3.1.0]hexane-6- carboxamide | 333.0 | C | E | F |
| 6 | 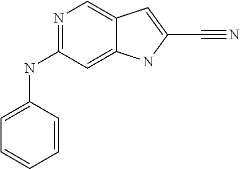 | 6-(phenylamino)-1H-pyrrolo[3,2- c]pyridine-2-carbonitrile | 235.0 | C | F | F |
| 7 | 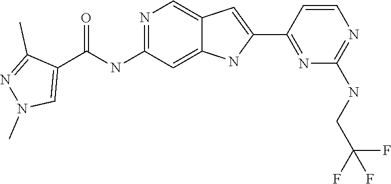 | 1,3-dimethyl-N-(2-(2-(2,2,2- trifluoroethylamino)pyrimidin-4-yl)- 1H-pyrrolo[3,2-c]pyridin-6-yl)-1H- pyrazole-4-carboxamide | 431.3 | C | F | F |
| 8 | 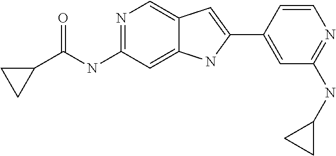 | N-(2-(2-(cyclopropylamino)pyridin- 4-yl)-1H-pyrrolo[3,2-c]pyridin-6- yl)cyclopropanecarboxamide | 334.0 | C | F | F |
| 9 | 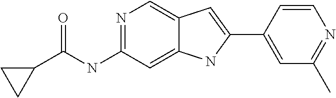 | N-(2-(2-methylpyridin-4-yl)-1H- pyrrolo[3,2-c]pyridin-6- yl)cyclopropanecarboxamide | 293.1 | C | F | F |
| 10 | 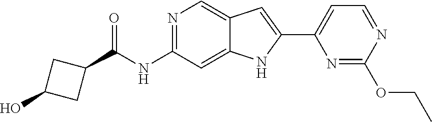 | (1s,3s)-N-(2-(2-ethoxypyrimidin-4- yl)-1H-pyrrolo[3,2-c]pyridin-6-yl)- 3- hydroxycyclobutanecarboxamide | 354.2 | C | F | F |
| 11 | 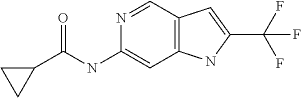 | N-(2-(trifluoromethyl)-1H- pyrrolo[3,2-c]pyridin-6- yl)cyclopropanecarboxamide | 270.2 | C | F | F |
| 12 | 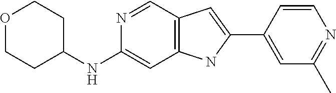 | 2-(2-methylpyridin-4-yl)-N- (tetrahydro-2H-pyran-4-yl)-1H- pyrrolo[3,2-c]pyridin-6-amine | 309.1 | B | F | F |
| 13 | 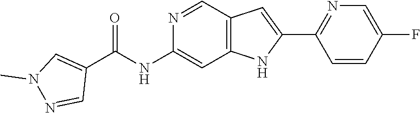 [SMILES: Cn1cc(C(=O)Nc2cc3[nH]c(-c4ccc(F)cn4)cc3cn2)cn1] | (R)-1-methyl-N-(2-(2-(1,1,1- trifluoropropan-2- ylamino)pyrimidin-4-yl)-1H- pyrrolo[3,2-c]pyridin-6-yl)-1H- pyrazole-4-carboxamide | 431.0 | B | E | F |
| 14 | 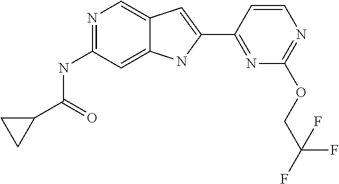 [SMILES: O=C(Nc1cc2[nH]c(-c3ccnc(OCC(F)(F)F)n3)cc2cn1)C1CC1] | N-(2-(2-(2,2,2- trifluoroethoxy)pyrimidin-4-yl)-1H- pyrrolo[3,2-c]pyridin-6- yl)cyclopropanecarboxamide | 378.2 | B | | |
| 15 | 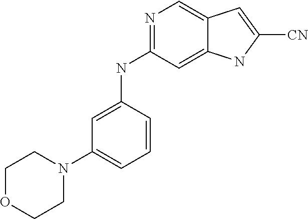 | 6-(3-morpholinophenylamino)-1H- pyrrolo[3,2-c]pyridine-2- carbonitrile | 320.0 | B | F | F |
| 16 | 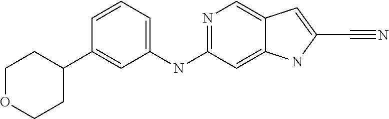 | 6-(3-(tetrahydro-2H-pyran-4- yl)phenylamino)-1H-pyrrolo[3,2- c]pyridine-2-carbonitrile | 319.5 | B | F | F |
| 17 | 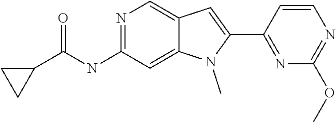 [SMILES: COc1nccc(-c2cc3cnc(NC(=O)C4CC4)cc3n2C)n1] | N-(2-(2-methoxypyrimidin-4-yl)-1- methyl-1H-pyrrolo[3,2-c]pyridin-6- yl)cyclopropanecarboxamide | 324.3 | B | D | F |
| 18 | 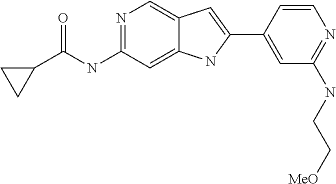 | N-(2-(2-(2- methoxyethylamino)pyridin-4-yl)- 1H-pyrrolo[3,2-c]pyridin-6- yl)cyclopropanecarboxamide | 352.0 | B | F | F |
| 19 | 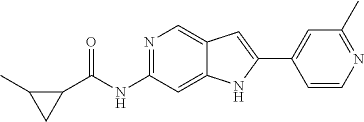 | 2-methyl-N-(2-(2-methylpyridin-4- yl)-1H-pyrrolo[3,2-c]pyridin-6- yl)cyclopropanecarboxamide | | B | F | F |
| 20 | 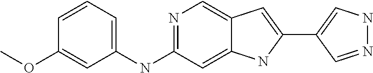 | N-(3-methoxyphenyl)-2-(1H- pyrazol-4-yl)-1H-pyrrolo[3,2- c]pyridin-6-amine | 305.7 | B | F | F |
| 21 | 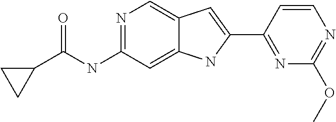 | N-(2-(2-methoxypyrimidin-4-yl)- 1H-pyrrolo[3,2-c]pyridin-6- yl)cyclopropanecarboxamide | 310.3 | B | F | F |
| 22 | 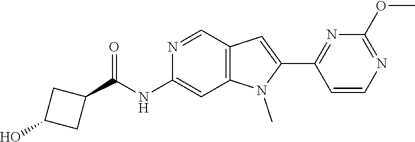 [SMILES: COc1nccc(-c2cc3cnc(NC(=O)[C@H]4C[C@H](O)C4)cc3n2C)n1] | (1r,3r)-3-hydroxy-N-(2-(2- methoxypyrimidin-4-yl)-1-methyl- 1H-pyrrolo[3,2-c]pyridin-6- yl)cyclobutanecarboxamide | | A | E | F |
| 23 | 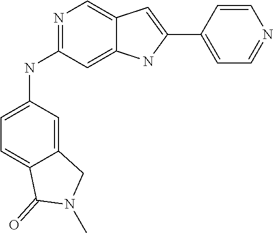 | 2-methyl-5-(2-(pyridin-4-yl)-1H- pyrrolo[3,2-c]pyridin-6- ylamino)isoindolin-1-one | 356.0 | A | F | F |
| 24 | 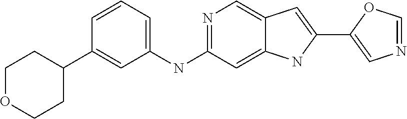 | 2-(oxazol-5-yl)-N-(3-(tetrahydro- 2H-pyran-4-yl)phenyl)-1H- pyrrolo[3,2-c]pyridin-6-amine | 361.5 | A | F | F |
| 25 | 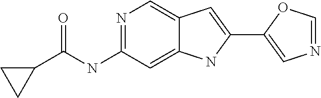 [SMILES: O=C(Nc1cc2[nH]c(-c3cnco3)cc2cn1)C1CC1] | N-(2-(oxazol-5-yl)-1H-pyrrolo[3,2- c]pyridin-6- yl)cyclopropanecarboxamide | 269.3 | A | D | F |
| 26 | 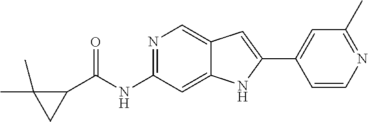 | N-(2-(2,6-difluoropyridin-4-yl)-1H- pyrrolo[3,2-c]pyridin-6- yl)cyclopropanecarboxamide | 315.1 | A | | |
| 27 | 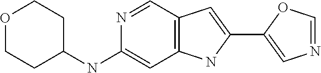 [SMILES: c1ncc(-c2cc3cnc(NC4CCOCC4)cc3[nH]2)o1] | 2-(oxazol-5-yl)-N-(tetrahydro-2H- pyran-4-yl)-1H-pyrrolo[3,2- c]pyridin-6-amine | 285.1 | A | | |
| 28 | 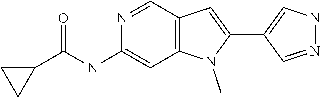 [SMILES: Cn1c(-c2cn[nH]c2)cc2cnc(NC(=O)C3CC3)cc21] | N-(1-methyl-2-(1H-pyrazol-4-yl)- 1H-pyrrolo[3,2-c]pyridin-6- yl)cyclopropanecarboxamide | 282.3 | A | | |
| 29 | 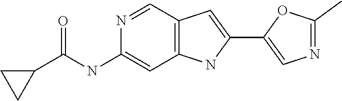 [SMILES: Cc1ncc(-c2cc3cnc(NC(=O)C4CC4)cc3[nH]2)o1] | N-(2-(2-methyloxazol-5-yl)-1H- pyrrolo[3,2-c]pyridin-6- yl)cyclopropanecarboxamide | 283.2 | A | | |
| 30 | 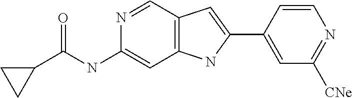 | N-(2-(2-cyanopyridin-4-yl)-1H- pyrrolo[3,2-c]pyridin-6- yl)cyclopropanecarboxamide | 304.2 | A | D | |
| 31 | 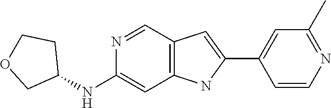 [SMILES: Cc1cc(-c2cc3cnc(N[C@H]4CCOC4)cc3[nH]2)ccn1] | (R)-2-(2-methylpyridin-4-yl)-N- (tetrahydrofuran-3-yl)-1H- pyrrolo[3,2-c]pyridin-6-amine | 295.1 | A | | |
| 32 | 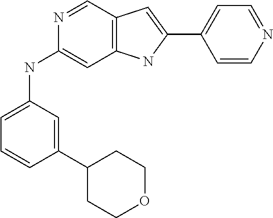 | 2-(pyridin-4-yl)-N-(3-(tetrahydro- 2H-pyran-4-yl)phenyl)-1H- pyrrolo[3,2-c]pyridin-6-amine | 371.0 | A | F | F |
| 33 | 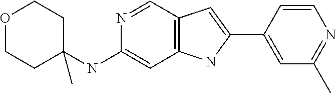 [SMILES: Cc1cc(-c2cc3cnc(NC4(C)CCOCC4)cc3[nH]2)ccn1] | 2-(2-methylpyridin-4-yl)-N-(4- methyltetrahydro-2H-pyran-4-yl)- 1H-pyrrolo[3,2-c]pyridin-6-amine | 323.1 | A | | |
| 34 | 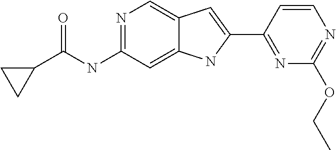 [SMILES: CCOc1nccc(-c2cc3cnc(NC(=O)C4CC4)cc3[nH]2)n1] | N-(2-(2-ethoxypyrimidin-4-yl)-1H- pyrrolo[3,2-c]pyridin-6- yl)cyclopropanecarboxamide | 324.3 | A | E | F |
| 35 | 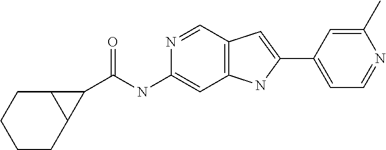 [SMILES: Cc1cc(-c2cc3cnc(NC(=O)C4C5CCCCC54)cc3[nH]2)ccn1] | N-(2-(2-methylpyridin-4-yl)-1H- pyrrolo[3,2-c]pyridin-6- y)bicyclo[4.1.0]heptane-7- carboxamide | | A | D | F |
| 36 | 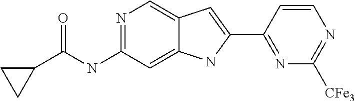 | N-(2-(2-(trifluoromethyl)pyrimidin- 4-yl)-1H-pyrrolo[3,2-c]pyridin-6- yl)cyclopropanecarboxamide | 348.0 | A | E | F |
| 37 | 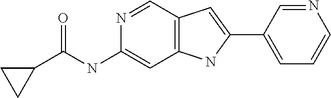 [SMILES: O=C(Nc1cc2[nH]c(-c3cccnc3)cc2cn1)C1CC1] | N-(2-(pyridin-3-yl)-1H-pyrrolo[3,2- c]pyridin-6- yl)cyclopropanecarboxamide | 279.2 | A | D | D |
| 38 | 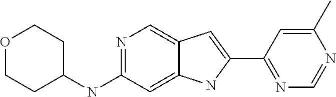 [SMILES: Cc1cc(-c2cc3cnc(NC4CCOCC4)cc3[nH]2)ncn1] | 2-(6-methylpyrimidin-4-yl)-N- (tetrahydro-2H-pyran-4-yl)-1H- pyrrolo[3,2-c]pyridin-6-amine | 310.2 | A | | |
| 39 | 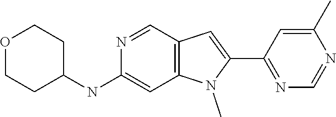 [SMILES: Cc1cc(-c2cc3cnc(NC4CCOCC4)cc3n2C)ncn1] | 1-methyl-2-(6-methylpyrimidin-4- yl)-N-(tetrahydro-2H-pyran-4-yl)- 1H-pyrrolo[3,2-c]pyridin-6-amine | 324.3 | A | | |
| 40 | 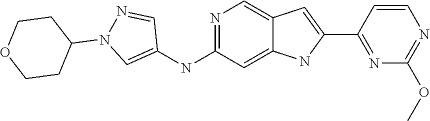 [SMILES: COc1nccc(-c2cc3cnc(Nc4cnn(C5CCOCC5)c4)cc3[nH]2)n1] | 2-(2-methoxypyrimidin-4-yl)-N-(1- (tetrahydro-2H-pyran-4-yl)-1H- pyrazol-4-yl)-1H-pyrrolo[3,2- c]pyridin-6-amine | 392.3 | A | E | F |
| 41 | 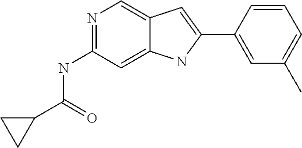 [SMILES: Cc1cccc(-c2cc3cnc(NC(=O)C4CC4)cc3[nH]2)c1] | N-(2-m-tolyl-1H-pyrrolo[3,2- c]pyridin-6- yl)cyclopropanecarboxamide | 292.2 | A | | |
| 42 | 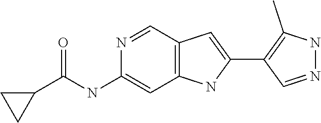 [SMILES: Cc1[nH]ncc1-c1cc2cnc(NC(=O)C3CC3)cc2[nH]1] | N-(2-(5-methyl-1H-pyrazol-4-yl)- 1H-pyrrolo[3,2-c]pyridin-6- yl)cyclopropanecarboxamide | 282.2 | A | | |
| 43 | 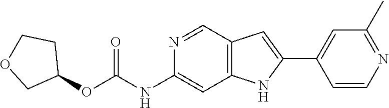 | (S)-tetrahydrofuran-3-yl 2-(2- methylpyridin-4-yl)-1H-pyrrolo[3,2- c]pyridin-6-ylcarbamate | | A | E | F |
| 44 | 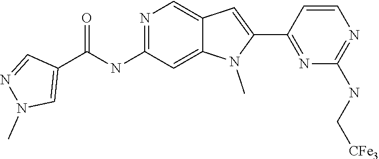 | 1-methyl-N-(1-methyl-2-(2-(2,2,2- trifluoroethylamino)pyrimidin-4-yl)- 1H-pyrrolo[3,2-c]pyridin-6-yl)-1H- pyrazole-4-carboxamide | 431.3 | A | D | F |
| 45 | 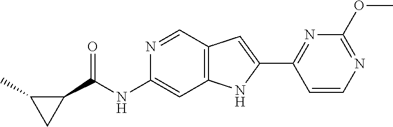 | (1R,2R)-N-(2-(2-methoxypyrimidin- 4-yl)-1H-pyrrolo[3,2-c]pyridin-6- yl)-2- methylcyclopropanecarboxamide | | A | D | F |
| 46 | 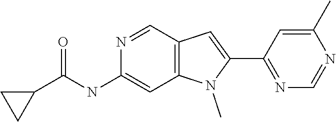 [SMILES: Cc1cc(-c2cc3cnc(NC(=O)C4CC4)cc3n2C)ncn1] | N-(1-methyl-2-(6-methylpyrimidin- 4-yl)-1H-pyrrolo[3,2-c]pyridin-6- yl)cyclopropanecarboxamide | 308.3 | A | | |
| 47 | 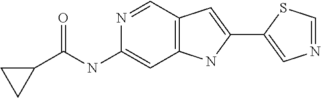 [SMILES: O=C(Nc1cc2[nH]c(-c3cncs3)cc2cn1)C1CC1] | N-(2-(thiazol-5-yl)-1H-pyrrolo[3,2- c]pyridin-6- yl)cyclopropanecarboxamide | 285.1 | A | | |
| 48 | 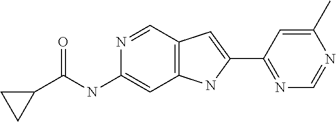 [SMILES: Cc1cc(-c2cc3cnc(NC(=O)C4CC4)cc3[nH]2)ncn1] | N-(2-(6-methylpyrimidin-4-yl)-1H- pyrrolo[3,2-c]pyridin-6- yl)cyclopropanecarboxamide | 294.3 | A | D | F |
| 49 | 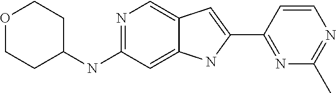 [SMILES: Cc1nccc(-c2cc3cnc(NC4CCOCC4)cc3[nH]2)n1] | 2-(2-methylpyrimidin-4-yl)-N- (tetrahydro-2H-pyran-4-yl)-1H- pyrrolo[3,2-c]pyridin-6-amine | 310.2 | A | | |
| 50 | 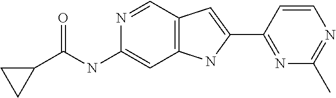 [SMILES: Cc1nccc(-c2cc3cnc(NC(=O)C4CC4)cc3[nH]2)n1] | N-(2-(2-methylpyrimidin-4-yl)-1H- pyrrolo[3,2-c]pyridin-6- yl)cyclopropanecarboxamide | 294.1 | A | D | F |
| 51 | 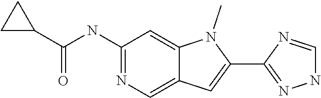 [SMILES: Cn1c(-c2nc[nH]n2)cc2cnc(NC(=O)C3CC3)cc21] | N-(1-methyl-2-(1H-1,2,4-triazol-3- yl)-1H-pyrrolo[3,2-c]pyridin-6- yl)cyclopropanecarboxamide | 283.2 | A | | |
| 52 | 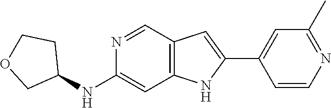 [SMILES: Cc1cc(-c2cc3cnc(N[C@@H]4CCOC4)cc3[nH]2)ccn1] | (S)-2-(2-methylpyridin-4-yl)-N- (tetrahydrofuran-3-yl)-1H- pyrrolo[3,2-c]pyridin-6-amine | 295.1 | A | | |
| 53 | 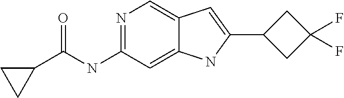 [SMILES: O=C(Nc1cc2[nH]c(C3CC(F)(F)C3)cc2cn1)C1CC1] | N-(2-(3,3-difluorocyclobutyl)-1H- pyrrolo[3,2-c]pyridin-6- yl)cyclopropanecarboxamide | 292.2 | A | | |
| 54 | 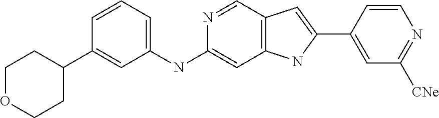 | 4-(6-(3-(tetrahydro-2H-pyran-4- yl)phenylamino)-1H-pyrrolo[3,2- c]pyridin-2-yl)picolinonitrile | 396.3 | A | D | F |
| 55 | 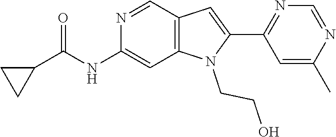 [SMILES: Cc1cc(-c2cc3cnc(NC(=O)C4CC4)cc3n2CCO)ncn1] | N-(1-(2-hydroxyethyl)-2-(6- methylpyrimidin-4-yl)-1H- pyrrolo[3,2-c]pyridin-6- yl)cyclopropanecarboxamide | 338.3 | A | | |
| 56 | 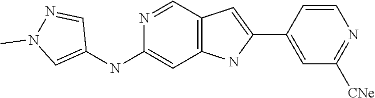 | 4-(6-(1-methyl-1H-pyrazol-4- ylamino)-1H-pyrrolo[3,2-c]pyridin- 2-yl)picolinonitrile | 316.2 | A | D | F |
| 57 | 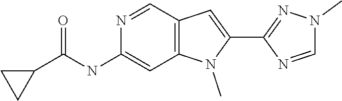 [SMILES: Cn1cnc(-c2cc3cnc(NC(=O)C4CC4)cc3n2C)n1] | N-(1-methyl-2-(1-methyl-1H-1,2,4- triazol-3-yl)-1H-pyrrolo[3,2- c]pyridin-6- yl)cyclopropanecarboxamide | 297.3 | A | | |
| 58 | 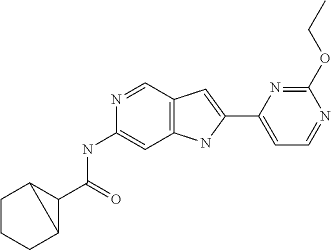 [SMILES: CCOc1nccc(-c2cc3cnc(NC(=O)C4C5CCCC54)cc3[nH]2)n1] | N-(2-(2-ethoxypyrimidin-4-yl)-1H- pyrrolo[3,2-c]pyridin-6- yl)bicyclo[3.1.0]hexane-6- carboxamide | 364.6 | A | D | F |
| 59 | 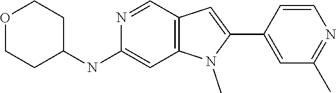 [SMILES: Cc1cc(-c2cc3cnc(NC4CCOCC4)cc3n2C)ccn1] | 1-methyl-2-(2-methylpyridin-4-yl)- N-(tetrahydro-2H-pyran-4-yl)-1H- pyrrolo[3,2-c]pyridin-6-amine | 323.1 | A | | |
| 60 | 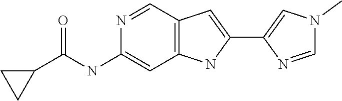 [SMILES: Cn1cnc(-c2cc3cnc(NC(=O)C4CC4)cc3[nH]2)c1] | N-(2-(1-methyl-1H-imidazol-4-yl)- 1H-pyrrolo[3,2-c]pyridin-6- yl)cyclopropanecarboxamide | 282.1 | A | | |
| 61 | 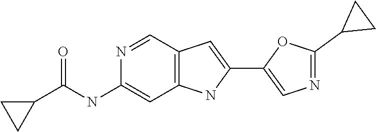 [SMILES: O=C(Nc1cc2[nH]c(-c3cnc(C4CC4)o3)cc2cn1)C1CC1] | N-(2-(2-cyclopropyloxazol-5-yl)- 1H-pyrrolo[3,2-c]pyridin-6- yl)cyclopropanecarboxamide | 309.1 | A | | |
| 62 | 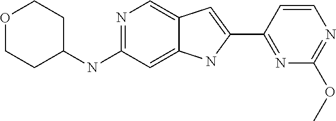 [SMILES: COc1nccc(-c2cc3cnc(NC4CCOCC4)cc3[nH]2)n1] | 2-(2-methoxypyrimidin-4-yl)-N- (tetrahydro-2H-pyran-4-yl)-1H- pyrrolo[3,2-c]pyridin-6-amine | 326.3 | A | | |
| 63 | 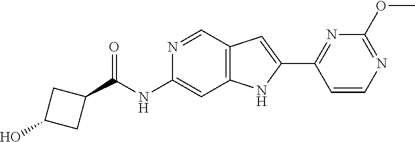 [SMILES: COc1nccc(-c2cc3cnc(NC(=O)[C@H]4C[C@H](O)C4)cc3[nH]2)n1] | (1r,3r)-3-hydroxy-N-(2-(2- methoxypyrimidin-4-yl)-1H- pyrrolo[3,2-c]pyridin-6- yl)cyclobutanecarboxamide | 340.4 | A | D | F |
| 64 | 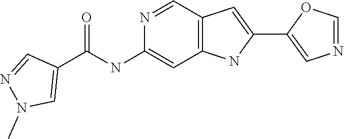 [SMILES: Cn1cc(C(=O)Nc2cc3[nH]c(-c4cnco4)cc3cn2)cn1] | 1-methyl-N-(2-(oxazol-5-yl)-1H- pyrrolo[3,2-c]pyridin-6-yl)-1H- pyrazole-4-carboxamide | 309.1 | A | D | E |
| 65 | 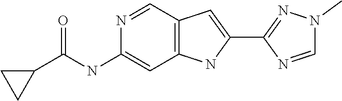 [SMILES: Cn1cnc(-c2cc3cnc(NC(=O)C4CC4)cc3[nH]2)n1] | N-(2-(1-methyl-1H-1,2,4-triazol-3- yl)-1H-pyrrolo[3,2-c]pyridin-6- yl)cyclopropanecarboxamide | 283.2 | A | | |
| 66 | 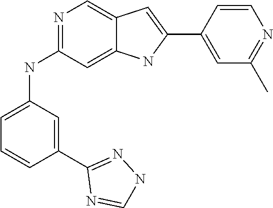 | N-(3-(1H-1,2,4-triazol-3- yl)phenyl)-2-(2-methylpyridin-4- yl)-1H-pyrrolo[3,2-c]pyridin-6- amine | 368.4 | A | F | F |
| 67 | 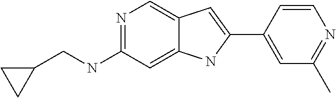 [SMILES: Cc1cc(-c2cc3cnc(NCC4CC4)cc3[nH]2)ccn1] | N-(cyclopropylmethyl)-2-(2- methylpyridin-4-yl)-1H-pyrrolo[3,2- c]pyridin-6-amine | 279.2 | A | | |
| 68 | 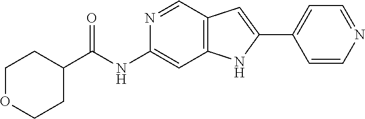 [SMILES: O=C(Nc1cc2[nH]c(-c3ccncc3)cc2cn1)C1CCOCC1] | N-(2-(pyridin-4-yl)-1H-pyrrolo[3,2- c]pyridin-6-yl)tetrahydro-2H- pyran-4-carboxamide | 323.0 | A | D | F |
| 69 | 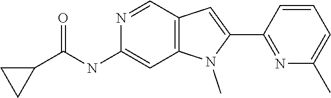 [SMILES: Cc1cccc(-c2cc3cnc(NC(=O)C4CC4)cc3n2C)n1] | N-(1-methyl-2-(6-methylpyridin-2- yl)-1H-pyrrolo[3,2-c]pyridin-6- yl)cyclopropanecarboxamide | 307.2 | A | | |
| 70 | 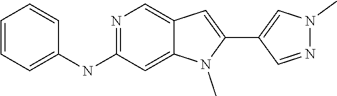 [SMILES: Cn1cc(-c2cc3cnc(Nc4ccccc4)cc3n2C)cn1] | 1-methyl-2-(1-methyl-1H-pyrazol- 4-yl)-N-phenyl-1H-pyrrolo[3,2- c]pyridin-6-amine | | A | E | F |
| 71 | 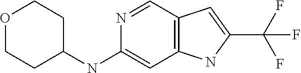 [SMILES: FC(F)(F)c1cc2cnc(NC3CCOCC3)cc2[nH]1] | N-(tetrahydro-2H-pyran-4-yl)-2- (trifluoromethyl)-1H-pyrrolo[3,2- c]pyridin-6-amine | 286.2 | A | | |
| 72 | 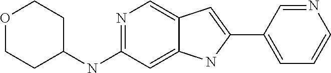 [SMILES: c1cncc(-c2cc3cnc(NC4CCOCC4)cc3[nH]2)c1] | 2-(pyridin-3-yl)-N-(tetrahydro-2H- pyran-4-yl)-1H-pyrrolo[3,2- c]pyridin-6-amine | 295.1 | A | D | D |
| 73 | 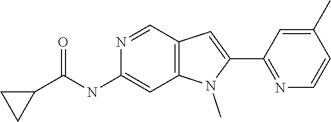 [SMILES: Cc1ccnc(-c2cc3cnc(NC(=O)C4CC4)cc3n2C)c1] | N-(1-methyl-2-(4-methylpyridin-2- yl)-1H-pyrrolo[3,2-c]pyridin-6- yl)cyclopropanecarboxamide | 307.1 | A | | |
| 74 | 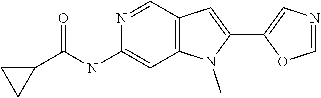 [SMILES: Cn1c(-c2cnco2)cc2cnc(NC(=O)C3CC3)cc21] | N-(1-methyl-2-(oxazol-5-yl)-1H- pyrrolo[3,2-c]pyridin-6- y)cyclopropanecarboxamide | 283.2 | A | | |
| 75 | 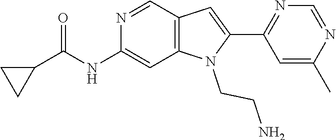 [SMILES: Cc1cc(-c2cc3cnc(NC(=O)C4CC4)cc3n2CCN)ncn1] | N-(1-(2-aminoethyl)-2-(6- methylpyrimidin-4-yl)-1H- pyrrolo[3,2-c]pyridin-6- yl)cyclopropanecarboxamide | 337.1 | A | | |
| 76 | 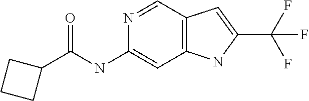 [SMILES: O=C(Nc1cc2[nH]c(C(F)(F)F)cc2cn1)C1CCC1] | N-(2-(trifluoromethyl)-1H- pyrrolo[3,2-c]pyridin-6- yl)cyclobutanecarboxamide | 284.3 | A | | |
| 77 | 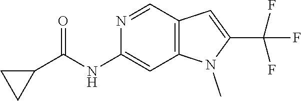 [SMILES: Cn1c(C(F)(F)F)cc2cnc(NC(=O)C3CC3)cc21] | N-(1-methyl-2-(trifluoromethyl)-1H- pyrrolo[3,2-c]pyridin-6- yl)cyclopropanecarboxamide | 284.2 | A | | |
| 78 | 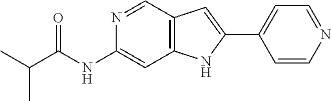 [SMILES: CC(C)C(=O)Nc1cc2[nH]c(-c3ccncc3)cc2cn1] | N-(2-(pyridin-4-yl)-1H-pyrrolo[3,2- c]pyridin-6-yl)isobutyramide | 281.0 | A | E | F |
| 79 | 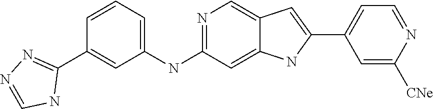 | 4-(6-(3-(4H-1,2,4-triazol-3- yl)phenylamino)-1H-pyrrolo[3,2- c]pyridin-2-yl)picolinonitrile | 379.0 | A | E | F |
| 80 | 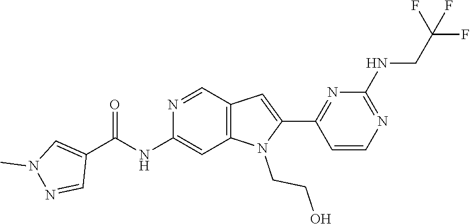 [SMILES: Cn1cc(C(=O)Nc2cc3c(cn2)cc(-c2ccnc(NCC(F)(F)F)n2)n3CCO)cn1] | N-(1-(2-hydroxyethyl)-2-(2-(2,2,2- trifluoroethylamino)pyrimidin-4-yl)- 1H-pyrrolo[3,2-c]pyridin-6-yl)-1- methyl-1H-pyrazole-4- carboxamide | 461.3 | A | D | E |
| 81 | 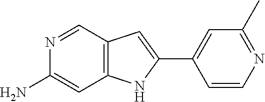 [SMILES: Cc1cc(-c2cc3cnc(N)cc3[nH]2)ccn1] | 2-(2-methylpyridin-4-yl)-1H- pyrrolo[3,2-c]pyridin-6-amine | 225.0 | A | D | E |
| 82 | 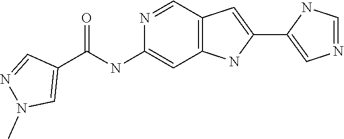 [SMILES: Cn1cc(C(=O)Nc2cc3[nH]c(-c4cnc[nH]4)cc3cn2)cn1] | N-(2-(1H-imidazol-5-yl)-1H- pyrrolo[3,2-c]pyridin-6-yl)-1- methyl-1H-pyrazole-4- carboxamide | 308.1 | A | D | E |
| 83 | 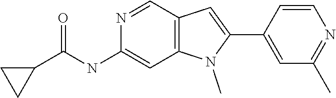 [SMILES: Cc1cc(-c2cc3cnc(NC(=O)C4CC4)cc3n2C)ccn1] | N-(1-methyl-2-(2-methylpyridin-4- yl)-1H-pyrrolo[3,2-c]pyridin-6- yl)cyclopropanecarboxamide | 307.1 | A | D | D |
| 84 | 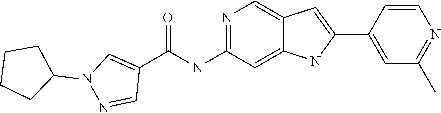 [SMILES: Cc1cc(-c2cc3cnc(NC(=O)c4cnn(C5CCCC5)c4)cc3[nH]2)ccn1] | 1-cyclopentyl-N-(2-(2- methylpyridin-4-yl)-1H-pyrrolo[3,2- c]pyridin-6-yl)-1H-pyrazole-4- carboxamide | 387.0 | A | F | D |
| 85 | 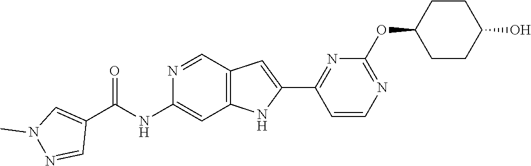 [SMILES: Cn1cc(C(=O)Nc2cc3[nH]c(-c4ccnc(O[C@H]5CC[C@H](O)CC5)n4)cc3cn2)cn1] | N-(2-(2-((1r,4r)-4- hydroxycyclohexyloxy)pyrimidin- 4-yl)-1H-pyrrolo[3,2-c]pyridin-6- yl)-1-methyl-1H-pyrazole-4- carboxamide | 434.0 | A | F | D |
| 86 | 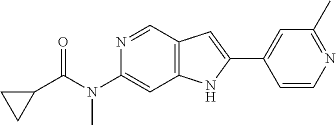 [SMILES: Cc1cc(-c2cc3cnc(N(C)C(=O)C4CC4)cc3[nH]2)ccn1] | N-methyl-N-(2-(2-methylpyridin-4- yl)-1H-pyrrolo[3,2-c]pyridin-6- yl)cyclopropanecarboxamide | 307.2 | A | E | D |
| 87 | 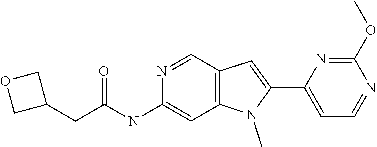 [SMILES: COc1nccc(-c2cc3cnc(NC(=O)CC4COC4)cc3n2C)n1] | N-(2-(2-methoxypyrimidin-4-yl)-1- methyl-1H-pyrrolo[3,2-c]pyridin-6- yl)-2-(oxetan-3-yl)acetamide | | A | E | D |
| 88 | 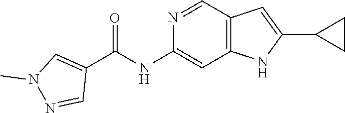 [SMILES: Cn1cc(C(=O)Nc2cc3[nH]c(C4CC4)cc3cn2)cn1] | N-(2-cyclopropyl-1H-pyrrolo[3,2- c]pyridin-6-yl)-1-methyl-1H- pyrazole-4-carboxamide | 282.0 | A | E | D |
| 89 | 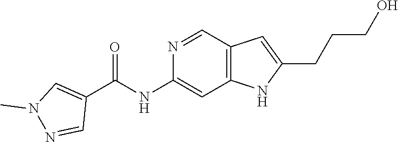 [SMILES: Cn1cc(C(=O)Nc2cc3[nH]c(CCCO)cc3cn2)cn1] | N-(2-(3-hydroxypropyl)-1H- pyrrolo[3,2-c]pyridin-6-yl)-1- methyl-1H-pyrazole-4- carboxamide | 300.0 | A | D | D |
| 90 | 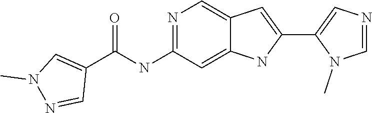 [SMILES: Cn1cc(C(=O)Nc2cc3[nH]c(-c4cncn4C)cc3cn2)cn1] | 1-methyl-N-(2-(1-methyl-1H- imidazol-5-yl)-1H-pyrrolo[3,2- c]pyridin-6-yl)-1H-pyrazole-4- carboxamide | 322.0 | A | E | D |
| 91 | 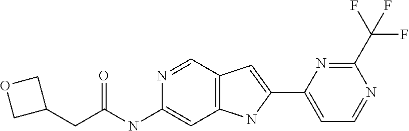 [SMILES: O=C(CC1COC1)Nc1cc2[nH]c(-c3ccnc(C(F)(F)F)n3)cc2cn1] | 2-(oxetan-3-yl)-N-(2-(2- (trifluoromethyl)pyrimidin-4-yl)-1H- pyrrolo[3,2-c]pyridin-6- yl)acetamide | | A | D | D |
| 92 | 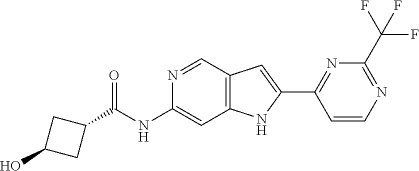 [SMILES: O=C(Nc1cc2[nH]c(-c3ccnc(C(F)(F)F)n3)cc2cn1)[C@H]1C[C@H](O)C1] | (1r,3r)-3-hydroxy-N-(2-(2- (trifluoromethyl)pyrimidin-4-yl)-1H- pyrrolo[3,2-c]pyridin-6- yl)cyclobutanecarboxamide | | A | E | D |
| 93 | 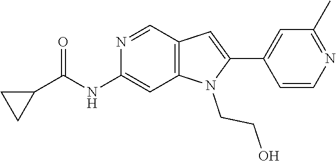 [SMILES: Cc1cc(-c2cc3cnc(NC(=O)C4CC4)cc3n2CCO)ccn1] | N-(1-(2-hydroxyethyl)-2-(2- methylpyridin-4-yl)-1H-pyrrolo[3,2- c]pyridin-6- yl)cyclopropanecarboxamide | 337.1 | A | D | D |
| 94 | 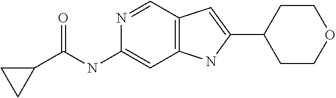 [SMILES: O=C(Nc1cc2[nH]c(C3CCOCC3)cc2cn1)C1CC1] | N-(2-(tetrahydro-2H-pyran-4-yl)- 1H-pyrrolo[3,2-c]pyridin-6- yl)cyclopropanecarboxamide | 286.2 | A | D | D |
| 95 | 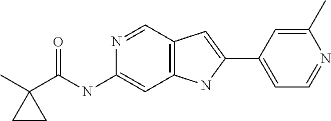 | 1-methyl-N-(2-(2-methylpyridin-4- yl)-1H-pyrrolo[3,2-c]pyridin-6- yl)cyclopropanecarboxamide | | A | | |
| 96 | 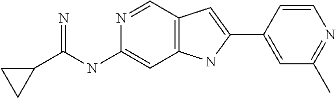 [SMILES: Cc1cc(-c2cc3cnc(NC(=N)C4CC4)cc3[nH]2)ccn1] | N-(2-(2-methylpyridin-4-yl)-1H- pyrrolo[3,2-c]pyridin-6- yl)cyclopropanecarboximidamide | 292.2 | A | | |
| 97 | 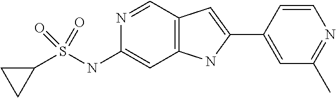 [SMILES: Cc1cc(-c2cc3cnc(NS(=O)(=O)C4CC4)cc3[nH]2)ccn1] | N-(2-(2-methylpyridin-4-yl)-1H- pyrrolo[3,2-c]pyridin-6- yl)cyclopropanesulfonamide | 329.2 | A | | |
| 98 | 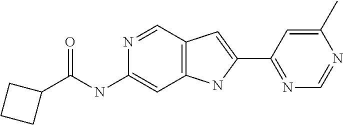 [SMILES: Cc1cc(-c2cc3cnc(NC(=O)C4CCC4)cc3[nH]2)ncn1] | N-(2-(6-methylpyrimidin-4-yl)-1H- pyrrolo[3,2-c]pyridin-6- yl)cyclobutanecarboxamide | 308.3 | A | | |
| 99 | 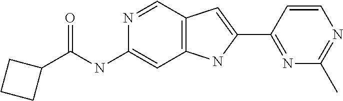 [SMILES: Cc1nccc(-c2cc3cnc(NC(=O)C4CCC4)cc3[nH]2)n1] | N-(2-(2-methylpyrimidin-4-yl)-1H- pyrrolo[3,2-c]pyridin-6- yl)cyclobutanecarboxamide | 308.3 | A | | |
| 100 | 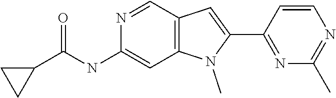 [SMILES: Cc1nccc(-c2cc3cnc(NC(=O)C4CC4)cc3n2C)n1] | N-(1-methyl-2-(2-methylpyrimidin- 4-yl)-1H-pyrrolo[3,2-c]pyridin-6- yl)cyclopropanecarboxamide | 308.3 | A | | |
| 101 | 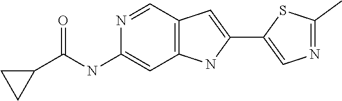 [SMILES: Cc1ncc(-c2cc3cnc(NC(=O)C4CC4)cc3[nH]2)s1] | N-(2-(2-methylthiazol-5-yl)-1H- pyrrolo[3,2-c]pyridin-6- yl)cyclopropanecarboxamide | 299.1 | A | | |
| 102 | 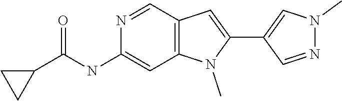 [SMILES: Cn1cc(-c2cc3cnc(NC(=O)C4CC4)cc3n2C)cn1] | N-(1-methyl-2-(1-methyl-1H- pyrazol-4-yl)-1H-pyrrolo[3,2- c]pyridin-6- yl)cyclopropanecarboxamide | 296.3 | A | | |
| 103 | 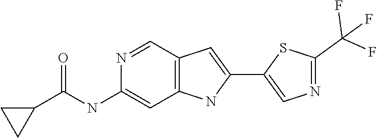 [SMILES: O=C(Nc1cc2[nH]c(-c3cnc(C(F)(F)F)s3)cc2cn1)C1CC1] | N-(2-(2-(trifluoromethyl)thiazol-5- yl)-1H-pyrrolo[3,2-c]pyridin-6- yl)cyclopropanecarboxamide | 353.1 | A | | |
| 104 | 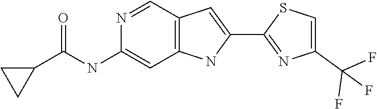 [SMILES: O=C(Nc1cc2[nH]c(-c3nc(C(F)(F)F)cs3)cc2cn1)C1CC1] | N-(2-(4-(trifluoromethyl)thiazol-2- yl)-1H-pyrrolo[3,2-c]pyridin-6- yl)cyclopropanecarboxamide | 353.1 | A | | |
| 105 | 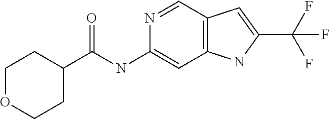 [SMILES: O=C(Nc1cc2[nH]c(C(F)(F)F)cc2cn1)C1CCOCC1] | N-(2-(trifluoromethyl)-1H- pyrrolo[3,2-c]pyridin-6- yl)tetrahydro-2H-pyran-4- carboxamide | 314.3 | A | | |
| 106 | 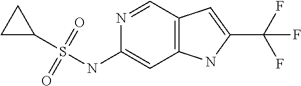 [SMILES: O=S(=O)(Nc1cc2[nH]c(C(F)(F)F)cc2cn1)C1CC1] | N-(2-(trifluoromethyl)-1H- pyrrolo[3,2-c]pyridin-6- yl)cyclopropanesulfonamide | 306.1 | A | | |
| 107 | 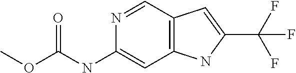 [SMILES: COC(=O)Nc1cc2[nH]c(C(F)(F)F)cc2cn1] | methyl 2-(trifluoromethyl)-1H- pyrrolo[3,2-c]pyridin-6- ylcarbamate | 260.1 | A | | |
| 108 | 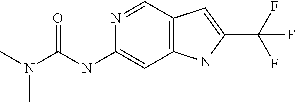 [SMILES: CN(C)C(=O)Nc1cc2[nH]c(C(F)(F)F)cc2cn1] | 1,1-dimethyl-3-(2-(trifluoromethyl)- 1H-pyrrolo[3,2-c]pyridin-6-yl)urea | 273.2 | A | | |
| 109 | 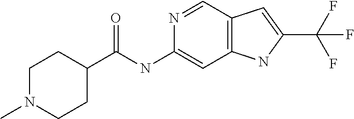 [SMILES: CN1CCC(C(=O)Nc2cc3[nH]c(C(F)(F)F)cc3cn2)CC1] | 1-methyl-N-(2-(trifluoromethyl)-1H- pyrrolo[3,2-c]pyridin-6- yl)piperidine-4-carboxamide | 327.1 | A | | |
| 110 | 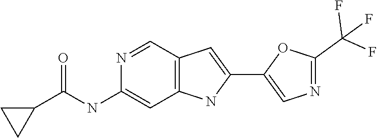 [SMILES: O=C(Nc1cc2[nH]c(-c3cnc(C(F)(F)F)o3)cc2cn1)C1CC1] | N-(2-(2-(trifluoromethyl)oxazol-5- yl)-1H-pyrrolo[3,2-c]pyridin-6- yl)cyclopropanecarboxamide | 337.2 | A | | |
| 111 | 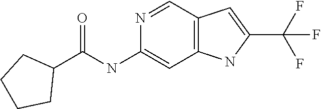 [SMILES: O=C(Nc1cc2[nH]c(C(F)(F)F)cc2cn1)C1CCCC1] | N-(2-(trifluoromethyl)-1H- pyrrolo[3,2-c]pyridin-6- yl)cyclopentanecarboxamide | 298.2 | A | | |
| 112 | 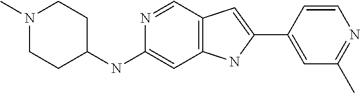 | N-(1-methylpiperidin-4-yl)-2-(2- methylpyridin-4-yl)-1H-pyrrolo[3,2- c]pyridin-6-amine | 322.2 | A | | |
| 113 | 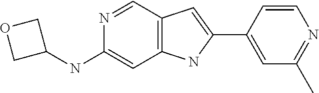 [SMILES: Cc1cc(-c2cc3cnc(NC4COC4)cc3[nH]2)ccn1] | 2-(2-methylpyridin-4-yl)-N- (oxetan-3-yl)-1H-pyrrolo[3,2- c]pyridin-6-amine | 281.1 | A | | |
| 114 | 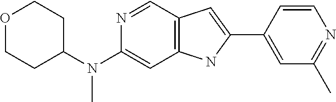 [SMILES: Cc1cc(-c2cc3cnc(N(C)C4CCOCC4)cc3[nH]2)ccn1] | N-methyl-2-(2-methylpyridin-4-yl)- N-(tetrahydro-2H-pyran-4-yl)-1H- pyrrolo[3,2-c]pyridin-6-amine | 323.1 | A | | |
| 115 | 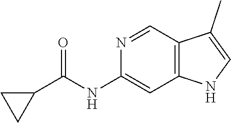 [SMILES: Cc1c[nH]c2cc(NC(=O)C3CC3)ncc12] | N-(3-methyl-1H-pyrrolo[3,2- c]pyridin-6- yl)cyclopropanecarboxamide | 216.2 | A | | |
| 116 | 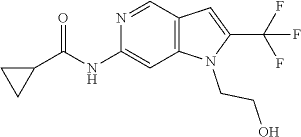 [SMILES: O=C(Nc1cc2c(cn1)cc(C(F)(F)F)n2CCO)C1CC1] | N-(1-(2-hydroxyethyl)-2- (trifluoromethyl)-1H-pyrrolo[3,2- c]pyridin-6- yl)cyclopropanecarobxamide | 314.2 | A | | |
| 117 | 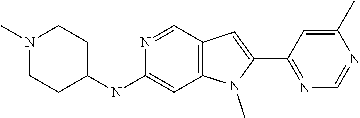 | 1-methyl-N-(1-methylpiperidin-4- yl)-2-(6-methylpyrimidin-4-yl)-1H- pyrrolo[3,2-c]pyridin-6-amine | 337.2 | A | | |
| 118 | 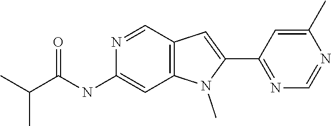 [SMILES: Cc1cc(-c2cc3cnc(NC(=O)C(C)C)cc3n2C)ncn1] | N-(1-methyl-2-(6-methylpyrimidin- 4-yl)-1H-pyrrolo[3,2-c]pyridin-6- yl)isobutyramide | 310.2 | A | | |
| 119 | 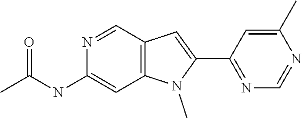 [SMILES: CC(=O)Nc1cc2c(cn1)cc(-c1cc(C)ncn1)n2C] | N-(1-methyl-2-(6-methylpyrimidin- 4-yl)-1H-pyrrolo[3,2-c]pyridin-6- yl)acetamide | 282.1 | A | | |
| 120 | 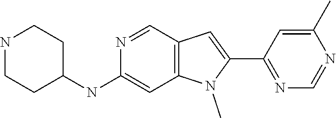 [SMILES: Cc1cc(-c2cc3cnc(NC4CCNCC4)cc3n2C)ncn1] | 1-methyl-2-(6-methylpyrimidin-4- yl)-N-(piperidin-4-yl)-1H- pyrrolo[3,2-c]pyridin-6-amine | 323.2 | A | | |
| 121 | 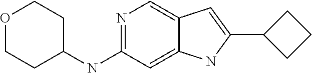 [SMILES: c1nc(NC2CCOCC2)cc2[nH]c(C3CCC3)cc12] | 2-cyclobutyl-N-(tetrahydro-2H- pyran-4-yl)-1H-pyrrolo[3,2- c]pyridin-6-amine | 272.3 | A | | |
| 122 | 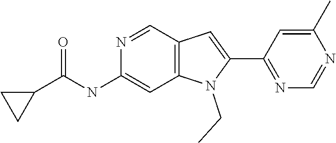 [SMILES: CCn1c(-c2cc(C)ncn2)cc2cnc(NC(=O)C3CC3)cc21] | N-(1-ethyl-2-(6-methylpyrimidin-4- yl)-1H-pyrrolo[3,2-c]pyridin-6- yl)cyclopropanecarboxamide | 322.3 | A | | |
| 123 | 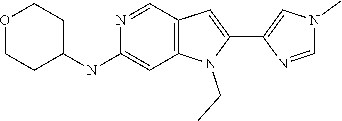 [SMILES: CCn1c(-c2cn(C)cn2)cc2cnc(NC3CCOCC3)cc21] | 2-(1-methyl-1H-imidazol-4-yl)-N- (tetrahydro-2H-pyran-4-yl)-1H- pyrrolo[3,2-c]pyridin-6-amine | 298.1 | A | | |
| 124 | 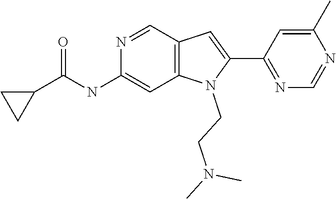 [SMILES: Cc1cc(-c2cc3cnc(NC(=O)C4CC4)cc3n2CCN(C)C)ncn1] | N-(1-(2-(dimethylamino)ethyl)-2- (6-methylpyrimidin-4-yl)-1H- pyrrolo[3,2-c]pyridin-6- yl)cyclopropanecarboxamide | 365.3 | A | | |
| 125 | 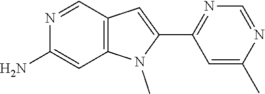 [SMILES: Cc1cc(-c2cc3cnc(N)cc3n2C)ncn1] | 1-methyl-2-(6-methylpyrimidin-4- y)-1H-pyrrolo[3,2-c]pyridin-6- amine | 240.1 | A | | |
| 126 | 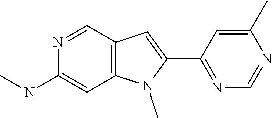 | N,1-dimethyl-2-(6-methylpyrimidin- 4-yl)-1H-pyrrolo[3,2-c]pyridin-6- amine | 254.3 | A | | |
| 127 | 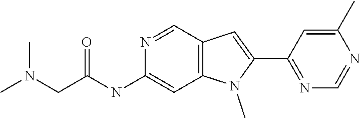 | 2-(dimethylamino)-N-(1-methyl-2- (6-methylpyrimidin-4-yl)-1H- pyrrolo[3,2-c]pyridin-6- yl)acetamide | 325.1 | A | | |
| 128 | 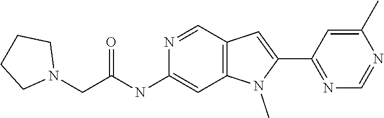 [SMILES: Cc1cc(-c2cc3cnc(NC(=O)CN4CCCC4)cc3n2C)ncn1] | N-(1-methyl-2-(6-methylpyrimidin- 4-yl)-1H-pyrrolo[3,2-c]pyridin-6- yl)-2-(pyrrolidin-1-yl)acetamide | 351.3 | A | | |
| 129 | 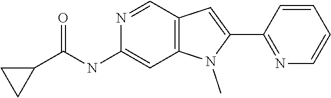 [SMILES: Cn1c(-c2ccccn2)cc2cnc(NC(=O)C3CC3)cc21] | N-(1-methyl-2-(pyridin-2-yl)-1H- pyrrolo[3,2-c]pyridin-6- yl)cyclopropanecarboxamide | 293.3 | A | | |
| 130 | 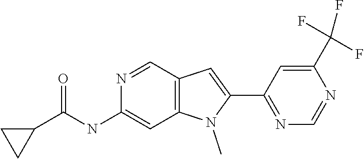 [SMILES: Cn1c(-c2cc(C(F)(F)F)ncn2)cc2cnc(NC(=O)C3CC3)cc21] | N-(1-methyl-2-(6- (trifluoromethyl)pyrimidin-4-yl)-1H- pyrrolo[3,2-c]pyridin-6- yl)cyclopropanecarboxamide | 362.3 | A | | |
| 131 | 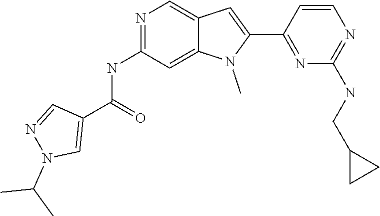 [SMILES: CC(C)n1cc(C(=O)Nc2cc3c(cn2)cc(-c2ccnc(NCC4CC4)n2)n3C)cn1] | N-(2-(2- (cyclopropylmethylamino)pyrimidin- 4-yl)-1-methyl-1H-pyrrolo[3,2- c]pyridin-6-yl)-1-isopropyl-1H- pyrazole-4-carboxmaide | 431.2 | | D | F |
| 132 | 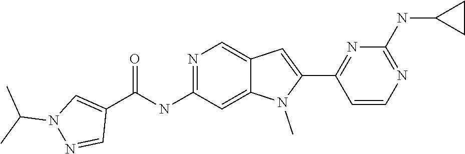 [SMILES: CC(C)n1cc(C(=O)Nc2cc3c(cn2)cc(-c2ccnc(NC4CC4)n2)n3C)cn1] | N-(2-(2- (cyclopropylamino)pyrimidin-4-yl)- 1-methyl-1H-pyrrolo[3,2-c]pyridin- 6-yl)-1-isopropyl-1H-pyrazole-4- carboxamide | 417.2 | | D | F |
| 133 | 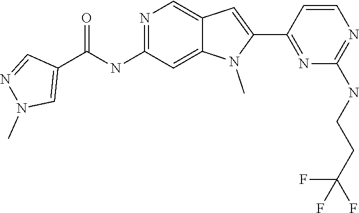 [SMILES: Cn1cc(C(=O)Nc2cc3c(cn2)cc(-c2ccnc(NCCC(F)(F)F)n2)n3C)cn1] | 1-methyl-N-(1-methyl-2-(2-(3,3,3- trifluoropropylamino)pyrimidin-4- yl)-1H-pyrrolo[3,2-c]pyridin-6-yl)- 1H-pyrazole-4-carboxamide | 445.2 | | D | F |
| 134 | 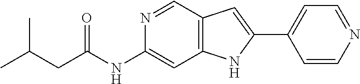 [SMILES: CC(C)CC(=O)Nc1cc2[nH]c(-c3ccncc3)cc2cn1] | 3-methyl-N-(2-(pyridin-4-yl)-1H- pyrrolo[3,2-c]pyridin-6- yl)butanamide | 295.0 | | D | F |
| 135 | 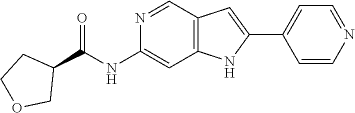 [SMILES: O=C(Nc1cc2[nH]c(-c3ccncc3)cc2cn1)[C@@H]1CCOC1] | (R)-N-(2-(pyridin-4-yl)-1H- pyrrolo[3,2-c]pyridin-6- yl)tetrahydrofuran-3- carboxamide | 309.0 | | D | F |
| 136 | 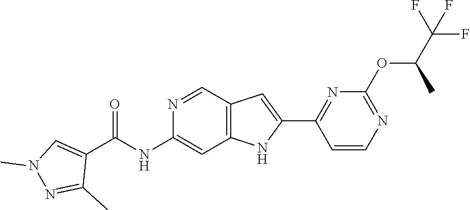 [SMILES: Cc1nn(C)cc1C(=O)Nc1cc2[nH]c(-c3ccnc(O[C@H](C)C(F)(F)F)n3)cc2cn1] | (S)-1,3-dimethyl-N-(2-(2-(1,1,1- trifluoropropan-2-yloxy)pyrimidin- 4-yl)-1H-pyrrolo[3,2-c]pyridin-6- yl)-1H-pyraozle-4-carboxamide | 446.1 | | D | F |
| 137 | 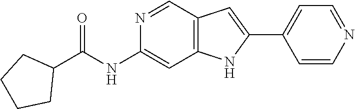 [SMILES: O=C(Nc1cc2[nH]c(-c3ccncc3)cc2cn1)C1CCCC1] | N-(2-(pyridin-4-yl)-1H-pyrrolo[3,2- c]pyridin-6- yl)cyclopentanecarboxamide | 307.0 | | D | F |
| 138 | 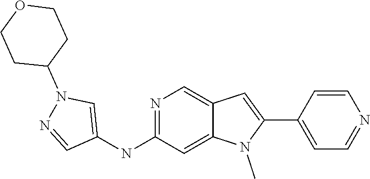 [SMILES: Cn1c(-c2ccncc2)cc2cnc(Nc3cnn(C4CCOCC4)c3)cc21] | 1-methyl-2-(pyridin-4-yl)-N-(1- (tetrahydro-2H-pyran-4-yl)-1H- pyrazol-4-yl)-1H-pyrrolo[3,2- c]pyridin-6-amine | 375.0 | | D | F |
| 139 | 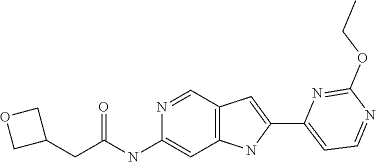 [SMILES: CCOc1nccc(-c2cc3cnc(NC(=O)CC4COC4)cc3[nH]2)n1] | N-(2-(2-ethoxypyrimidin-4-yl)-1H- pyrrolo[3,2-c]pyridin-6-yl)-2- (oxetan-3-yl)acetamide | 354.2 | | D | F |
| 140 | 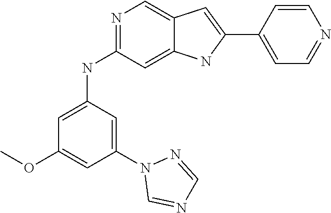 [SMILES: COc1cc(Nc2cc3[nH]c(-c4ccncc4)cc3cn2)cc(-n2cncn2)c1] | N-(3-methoxy-5-(1H-1,2,4-triazol- 1-yl)phenyl)-2-(pyridin-4-yl)-1H- pyrrolo[3,2-c]pyridin-6-amine | 384.0 | | D | F |
| 141 | 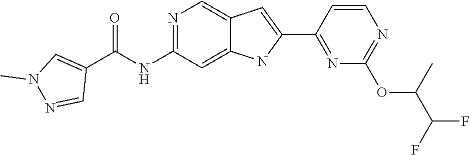 [SMILES: CC(Oc1nccc(-c2cc3cnc(NC(=O)c4cnn(C)c4)cc3[nH]2)n1)C(F)F] | N-(2-(2-(1,1-difluoropropan-2- yloxy)pyrimidin-4-yl)-1H- pyrrolo[3,2-c]pyridin-6-yl)-1- methyl-1H-pyrazole-4- carboxamide | 414.1 | | D | F |
| 142 | 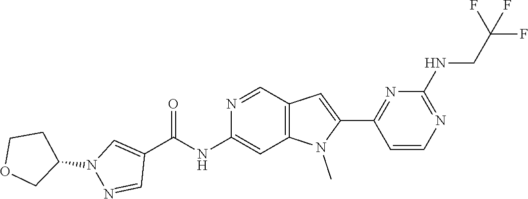 | (R)-N-(1-methyl-2-(2-(2,2,2- trifluoroethylamino)pyrimidin-4-yl)- 1H-pyrrolo[3,2-c]pyridin-6-yl)-1- (tetrahydrofuran-3-yl)-1H- pyrazole-4-carboxamide | 487.3 | | F | F |
| 143 | 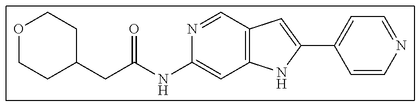 [SMILES: O=C(CC1CCOCC1)Nc1cc2[nH]c(-c3ccncc3)cc2cn1] | N-(2-(pyridin-4-yl)-1H-pyrrolo[3,2- c]pyridin-6-yl)-2-(tetrahydro-2H- pyran-4-yl)acetamide | 337.0 | | D | F |
| 144 | 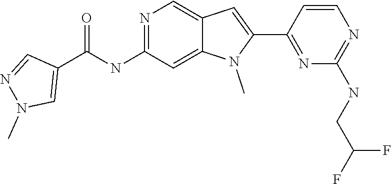 [SMILES: Cn1cc(C(=O)Nc2cc3c(cn2)cc(-c2ccnc(NCC(F)F)n2)n3C)cn1] | N-(2-(2-(2,2- difluroethylamino)pyrimidin-4-yl)- 1-methyl-1H-pyrrolo[3,2-c]pyridin- 6-yl)-1-methyl-1H-pyrazole-4- carboxmaide | 413.3 | | D | F |
| 145 | 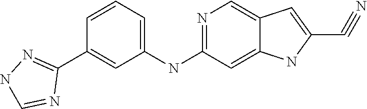 [SMILES: N#Cc1cc2cnc(Nc3cccc(-c4nc[nH]n4)c3)cc2[nH]1] | 6-(3-(1H-1,2,4-triazol-3- yl)phenylamino)-1H-pyrrolo[3,2- c]pyridine-2-carbonitrile | 302.3 | | D | F |
| 146 | 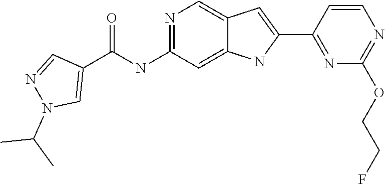 [SMILES: CC(C)n1cc(C(=O)Nc2cc3[nH]c(-c4ccnc(OCCF)n4)cc3cn2)cn1] | N-(2-(2-(2-fluoroethoxy)pyrimidin- 4-yl)-1H-pyrrolo[3,2-c]pyridin-6- yl)-1-isopropyl-1H-pyrazole-4- carboxamide | 410.1 | | D | F |
| 147 | 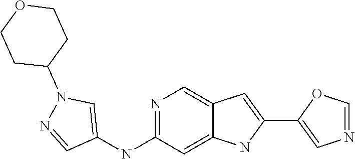 [SMILES: c1ncc(-c2cc3cnc(Nc4cnn(C5CCOCC5)c4)cc3[nH]2)o1] | 2-(oxazol-5-yl)-N-(1-(tetrahydro- 2H-pyran-4-yl)-1H-pyrazol-4-yl)- 1H-pyrrolo[3,2-c]pyridin-6-amine | 351.2 | | D | F |
| 148 | 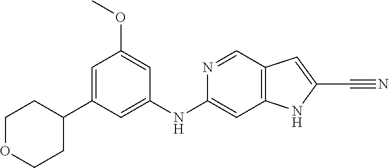 [SMILES: COc1cc(Nc2cc3[nH]c(C#N)cc3cn2)cc(C2CCOCC2)c1] | 6-(3-methoxy-5-(tetrahydro-2H- pyran-4-yl)phenylamino)-1H- pyrrolo[3,2-c]pyridine-2- carbonitrile | 349.0 | | D | F |
| 149 | 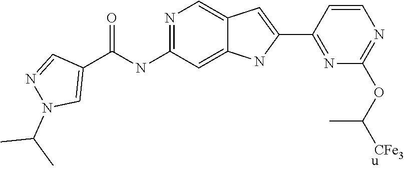 | 1-isopropyl-N-(2-(2-(1,1,1- trifluoropropan-2-yloxy)pyrimidin- 4-yl)-1H-pyrrolo[3,2-c]pyridin-6- yl)-1H-pyrazole-4-carboxamide | 460.0 | | D | F |
| 150 | 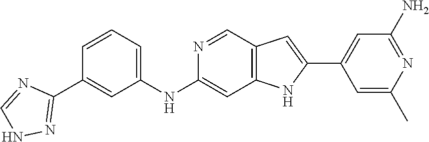 [SMILES: Cc1cc(-c2cc3cnc(Nc4cccc(-c5nc[nH]n5)c4)cc3[nH]2)cc(N)n1] | N-(3-(1H-1,2,4-triazol-3- yl)phenyl)-2-(2-amino-6- methylpyridin-4-yl)-1H-pyrrolo[3,2- c]pyridin-6-amine | 383.0 | | D | F |
| 151 | 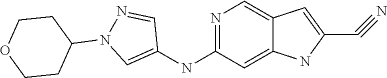 [SMILES: N#Cc1cc2cnc(Nc3cnn(C4CCOCC4)c3)cc2[nH]1] | 6-(1-(tetrahydro-2H-pyran-4-yl)- 1H-pyrazol-4-ylamino)-1H- pyrrolo[3,2-c]pyridine-2- carbonitrile | 308.9 | | D | F |
| 152 | 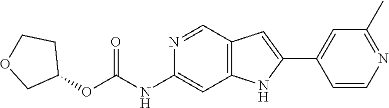 [SMILES: Cc1cc(-c2cc3cnc(NC(=O)O[C@H]4CCOC4)cc3[nH]2)ccn1] | (R)-tetrahydrofuran-3-yl 2-(2- methylpyridin-4-yl)-1H-pyrrolo[3,2- c]pyridin-6-ylcarbamate | | | D | F |
| 153 | 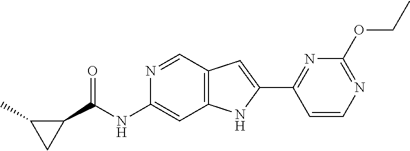 [SMILES: CCOc1nccc(-c2cc3cnc(NC(=O)[C@H]4C[C@@H]4C)cc3[nH]2)n1] | (1R,2R)-N-(2-(2-ethoxypyrimidin- 4-yl)-1H-pyrrolo[3,2-c]pyridin-6- yl)-2- methylcyclopropanecarboxamide | | | D | F |
| 154 | 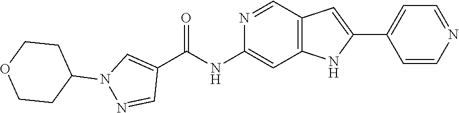 [SMILES: O=C(Nc1cc2[nH]c(-c3ccncc3)cc2cn1)c1cnn(C2CCOCC2)c1] | N-(2-(pyridin-4-yl)-1H-pyrrolo[3,2- c]pyridin-6-yl)-1-(tetrahydro-2H- pyran-4-yl)-1H-pyrazole-4- carboxamide | 389.0 | | D | F |
| 155 | 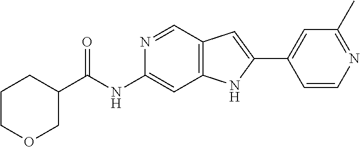 [SMILES: Cc1cc(-c2cc3cnc(NC(=O)C4CCCOC4)cc3[nH]2)ccn1] | N-(2-(2-methylpyridin-4-yl)-1H- pyrrolo[3,2-c]pyridin-6- yl)tetrahydro-2H-pyran-3- carboxamide | 337.0 | | D | F |
| 156 | 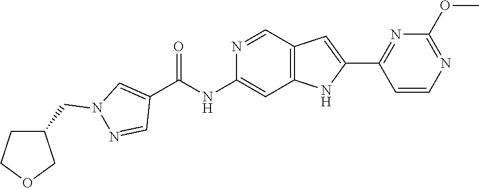 [SMILES: COc1nccc(-c2cc3cnc(NC(=O)c4cnn(C[C@H]5CCOC5)c4)cc3[nH]2)n1] | (S)-N-(2-(2-methoxypyrimidin-4- yl)-1H-pyrrolo[3,2-c]pyridin-6-yl)- 1-((tetrahydrofuran-3-yl)methyl)- 1H-pyrazole-4-carboxamide | 420.0 | | D | F |
| 157 | 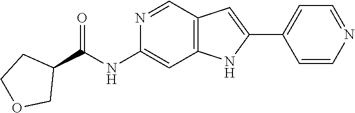 [SMILES: O=C(Nc1cc2[nH]c(-c3ccncc3)cc2cn1)[C@@H]1CCOC1] | (S)-N-(2-(pyridin-4-yl)-1H- pyrrolo[3,2-c]pyridin-6- yl)tetrahydrofuran-3- carboxamide | 309.0 | | D | F |
| 158 | 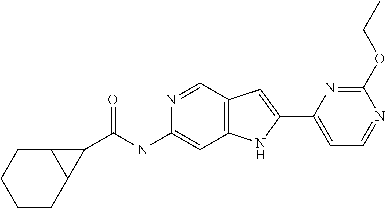 [SMILES: CCOc1nccc(-c2cc3cnc(NC(=O)C4C5CCCCC54)cc3[nH]2)n1] | N-(2-(2-ethoxypyrimidin-4-yl)-1H- pyrrolo[3,2-c]pyridin-6- yl)bicyclo[4.1.0]heptane-7- carboxamide | 378.2 | | D | F |
| 159 | 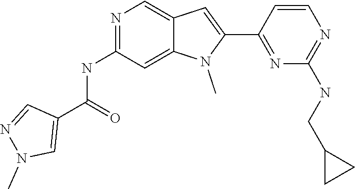 [SMILES: Cn1cc(C(=O)Nc2cc3c(cn2)cc(-c2ccnc(NCC4CC4)n2)n3C)cn1] | N-(2-(2- (cyclopropylmethylamino)pyrimidin- 4-yl)-1-methyl-1H-pyrrolo[3,2- c]pyridin-6-yl)-1-methyl-1H- pyrazole-4-carboxamide | 403.2 | | D | F |
| 160 | 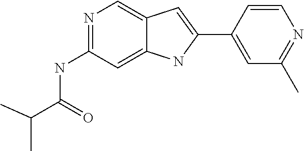 [SMILES: Cc1cc(-c2cc3cnc(NC(=O)C(C)C)cc3[nH]2)ccn1] | N-(2-(2-methylpyridin-4-yl)-1H- pyrrolo[3,2-c]pyridin-6- yl)isobutyramide | 295.0 | | D | F |
| 161 | 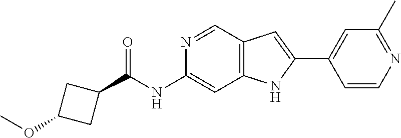 | (1r,3r)-3-methoxy-N-(2-(2- methylpyridin-4-yl)-1H-pyrrolo[3,2- c]pyridin-6- yl)cyclobutanecarboxamide | 337.0 | | D | F |
| 162 | 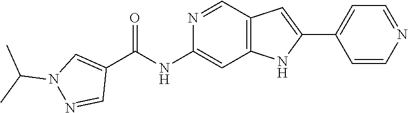 [SMILES: CC(C)n1cc(C(=O)Nc2cc3[nH]c(-c4ccncc4)cc3cn2)cn1] | 1-isopropyl-N-(2-(pyridin-4-yl)-1H- pyrrolo[3,2-c]pyridin-6-yl)-1H- pyrazole-4-carboxamide | 347.0 | | D | F |
| 163 | 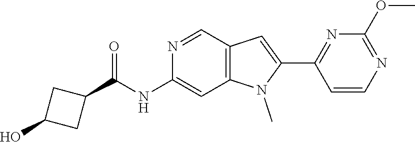 [SMILES: COc1nccc(-c2cc3cnc(NC(=O)[C@H]4C[C@@H](O)C4)cc3n2C)n1] | (1s,3s)-3-hydroxy-N-(2-(2- methoxypyrimidin-4-yl)-1-methyl- 1H-pyrrolo[3,2-c]pyridin-6- yl)cyclobutanecarboxamide | | | D | F |
| 164 | 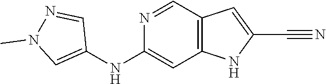 [SMILES: Cn1cc(Nc2cc3[nH]c(C#N)cc3cn2)cn1] | 6-(1-methyl-1H-pyrazol-4- ylamino)-1H-pyrrolo[3,2- c]pyridine-2-carbonitrile | 239.0 | | D | F |
| 165 | 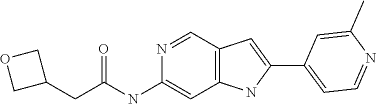 [SMILES: Cc1cc(-c2cc3cnc(NC(=O)CC4COC4)cc3[nH]2)ccn1] | N-(2-(2-methylpyridin-4-yl)-1H- pyrrolo[3,2-c]pyridin-6-yl)-2- (oxetan-3-yl)acetamide | 323.2 | | D | F |
| 166 | 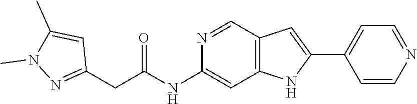 [SMILES: Cc1cc(CC(=O)Nc2cc3[nH]c(-c4ccncc4)cc3cn2)nn1C] | 1,5-dimethyl-N-(2-(pyridin-4-yl)- 1H-pyrrolo[3,2-c]pyridin-6-yl)-1H- pyrazole-4-carboxamide | 333.0 | | D | F |
| 167 | 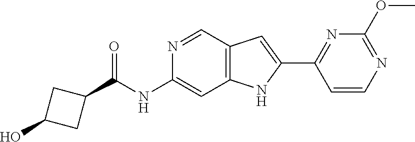 [SMILES: COc1nccc(-c2cc3cnc(NC(=O)[C@H]4C[C@@H](O)C4)cc3[nH]2)n1] | (1s,3s)-3-hydroxy-N-(2-(2- methoxypyrimidin-4-yl)-1H- pyrrolo[3,2-c]pyridin-6- yl)cyclobutanecarboxamide | 340.0 | | E | F |
| 168 | 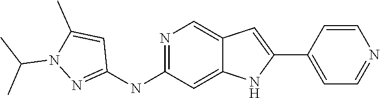 [SMILES: Cc1cc(Nc2cc3[nH]c(-c4ccncc4)cc3cn2)nn1C(C)C] | N-(1-isopropyl-5-methyl-1H- pyrazol-3-yl)-2-(pyridin-4-yl)-1H- pyrrolo[3,2-c]pyridin-6-amine | 333.5 | | E | F |
| 169 | 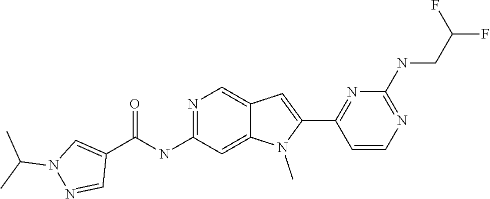 [SMILES: CC(C)n1cc(C(=O)Nc2cc3c(cn2)cc(-c2ccnc(NCC(F)F)n2)n3C)cn1] | N-(2-(2-(2,2- difluoroethylamino)pyrimidin-4-yl)- 1-methyl-1H-pyrrolo[3,2-c]pyridin- 6-yl)-1-isopropyl-1H-pyrazole-4- carboxamide | 441.2 | | D | F |
| 170 | 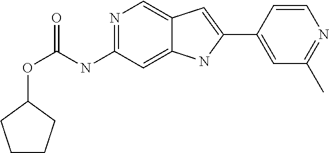 [SMILES: Cc1cc(-c2cc3cnc(NC(=O)OC4CCCC4)cc3[nH]2)ccn1] | cyclopentyl 2-(2-methylpyridin-4- yl)-1H-pyrrolo[3,2-c]pyridin-6- ylcarbamate | 337.0 | | D | F |
| 171 | 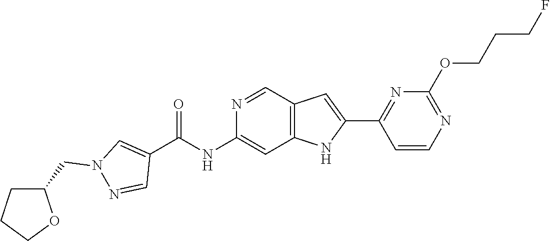 [SMILES: O=C(Nc1cc2[nH]c(-c3ccnc(OCCCF)n3)cc2cn1)c1cnn(C[C@H]2CCCO2)c1] | (S)-N-(2-(2-(3- fluoropropoxy)pyrimidin-4-yl)-1H- pyrrolo[3,2-c]pyridin-6-yl)-1- ((tetrahydrofuran-2-yl)methyl)-1H- pyrazole-4-carboxamide | 466.0 | | D | F |
| 172 | 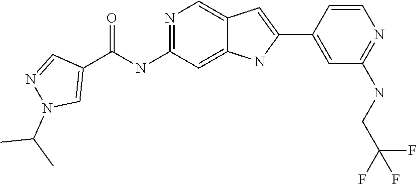 | 1-isopropyl-N-(2-(2-(2,2,2- trifluoroethylamino)pyrimidin-4-yl)- 1H-pyrrolo[3,2-c]pyridin-6-yl)-1H- pyrazole-4-carboxamide | 445.0 | | F | F |
| 173 | 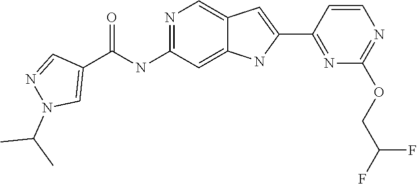 [SMILES: CC(C)n1cc(C(=O)Nc2cc3[nH]c(-c4ccnc(OCC(F)F)n4)cc3cn2)cn1] | N-(2-(2-(2,2- difluoroethoxy)pyrimidin-4-yl)-1H- pyrrolo[3,2-c]pyridin-6-yl)-1- isopropyl-1H-pyrazole-4- carboxamide | 428.1 | | D | F |
| 174 | 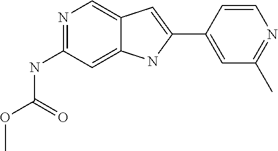 [SMILES: COC(=O)Nc1cc2[nH]c(-c3ccnc(C)c3)cc2cn1] | methyl 2-(2-methylpyridin-4-yl)- 1H-pyrrolo[3,2-c]pyridin-6- ylcarbamate | 283.0 | | D | F |
| 175 | 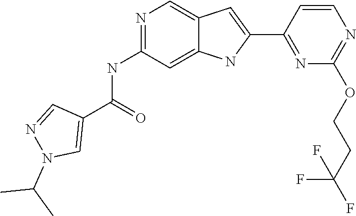 [SMILES: CC(C)n1cc(C(=O)Nc2cc3[nH]c(-c4ccnc(OCCC(F)(F)F)n4)cc3cn2)cn1] | 1-isopropyl-N-(2-(2-(3,3,3- trifluoropropoxy)pyrimidin-4-yl)- 1H-pyrrolo[3,2-c]pyridin-6-yl)-1H- pyrazole-4-carboxamide | 460.0 | | D | F |
| 176 | 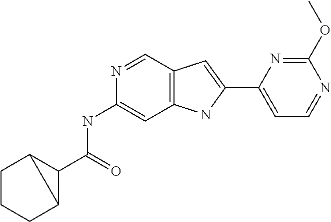 [SMILES: COc1nccc(-c2cc3cnc(NC(=O)C4C5CCCC54)cc3[nH]2)n1] | N-(2-(2-methoxypyrimidin-4-yl)- 1H-pyrrolo[3,2-c]pyridin-6- yl)bicyclo[3.1.0]hexane-6- carboxamide | 350.2 | | D | F |
| 177 | 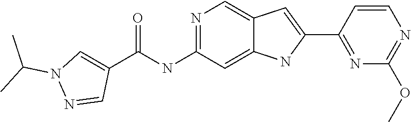 [SMILES: COc1nccc(-c2cc3cnc(NC(=O)c4cnn(C(C)C)c4)cc3[nH]2)n1] | 1-isopropyl-N-(2-(2- methoxypyrimidin-4-yl)-1H- pyrrolo[3,2-c]pyridin-6-yl)-1H- pyrazole-4-carboxmaide | 378.0 | | D | F |
| 178 | 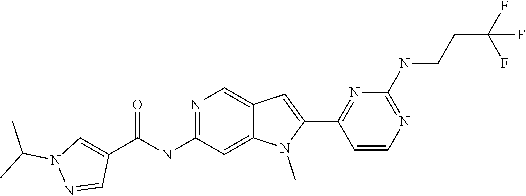 [SMILES: CC(C)n1cc(C(=O)Nc2cc3c(cn2)cc(-c2ccnc(NCCC(F)(F)F)n2)n3C)cn1] | 1-isopropyl-N-(1-methyl-2-(2- (3,3,3- trifluoropropylamino)pyrimidin-4- yl)-1H-pyrrolo[3,2-c]pyridin-6-yl)- 1H-pyrazole-4-carboxamide | 473.1 | | D | F |
| 179 | 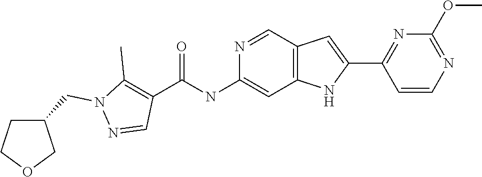 [SMILES: COc1nccc(-c2cc3cnc(NC(=O)c4cnn(C[C@H]5CCOC5)c4C)cc3[nH]2)n1] | (S)-N-(2-(2-methoxypyrimidin-4- yl)-1H-pyrrolo[3,2-c]pyridin-6-yl)- 5-methyl-1-((tetrahydrofuran-3- yl)methyl)-1H-pyrazole-4- carboxamide | 434.0 | | D | F |
| 180 | 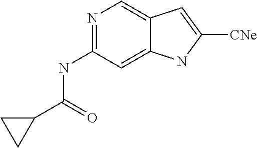 | N-(2-cyano-1H-pyrrolo[3,2- c]pyridin-6- yl)cyclopropanecarboxamide | 227.2 | | D | F |
| 181 | 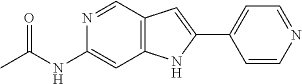 [SMILES: CC(=O)Nc1cc2[nH]c(-c3ccncc3)cc2cn1] | N-(2-(pyridin-4-yl)-1H-pyrrolo[3,2- c]pyridin-6-yl)acetamide | 253.0 | | D | F |
| 182 | 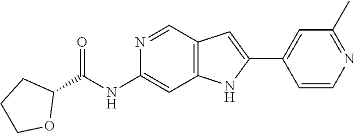 [SMILES: Cc1cc(-c2cc3cnc(NC(=O)[C@H]4CCCO4)cc3[nH]2)ccn1] | (S)-N-(2-(2-methylpyridin-4-yl)- 1H-pyrrolo[3,2-c]pyridin-6- yl)tetrahydrofuran-2- carboxamide | | | D | F |
| 183 | 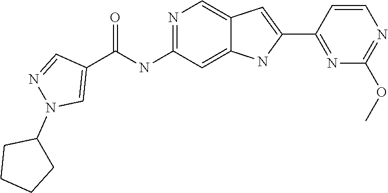 [SMILES: COc1nccc(-c2cc3cnc(NC(=O)c4cnn(C5CCCC5)c4)cc3[nH]2)n1] | 1-cyclopentyl-N-(2-(2- methoxypyrimidin-4-yl)-1H- pyrrolo[3,2-c]pyridin-6-yl)-1H- pyrazole-4-carboxamide | 404.0 | | D | F |
| 184 | 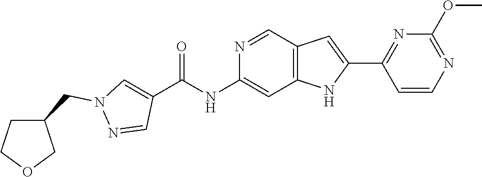 [SMILES: COc1nccc(-c2cc3cnc(NC(=O)c4cnn(C[C@@H]5CCOC5)c4)cc3[nH]2)n1] | (R)-N-(2-(2-methoxypyrimidin-4- yl)-1H-pyrrolo[3,2-c]pyridin-6-yl)- 1-((tetrahydrofuran-3-yl)methyl)- 1H-pyrazole-4-carboxamide | 420.0 | | D | F |
| 185 | 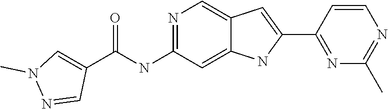 | 1-methyl-N-(2-(2-methylpyrimidin- 4-yl)-1H-pyrrolo[3,2-c]pyridin-6- yl)-1H-pyrazole-4-carboxamide | 333.9 | | D | F |
| 186 | 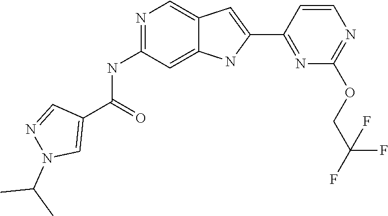 [SMILES: CC(C)n1cc(C(=O)Nc2cc3[nH]c(-c4ccnc(OCC(F)(F)F)n4)cc3cn2)cn1] | 1-isopropyl-N-(2-(2-(2,2,2- trifluoroethoxy)pyrimidin-4-yl)-1H- pyrrolo[3,2-c]pyridin-6-yl)-1H- pyrazole-4-carboxamide | 446.1 | | D | F |
| 187 | 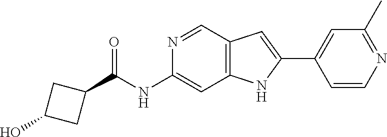 [SMILES: Cc1cc(-c2cc3cnc(NC(=O)[C@H]4C[C@H](O)C4)cc3[nH]2)ccn1] | (1r,3r)-3-hydroxy-N-(2-(2- methylpyridin-4-yl)-1H-pyrrolo[3,2- c]pyridin-6- yl)cyclobutanecarboxamide | | | E | F |
| 188 | 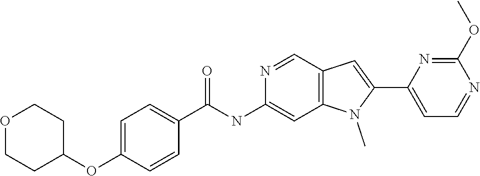 [SMILES: COc1nccc(-c2cc3cnc(NC(=O)c4ccc(OC5CCOCC5)cc4)cc3n2C)n1] | N-(2-(2-methoxypyrimidin-4-yl)-1- methyl-1H-pyrrolo[3,2-c]pyridin-6- yl)-4-(tetrahydro-2H-pyran-4- yloxy)benzamide | 460.2 | | D | F |
| 189 | 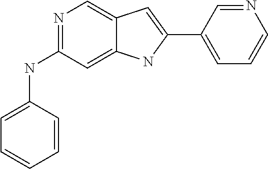 [SMILES: c1ccc(Nc2cc3[nH]c(-c4cccnc4)cc3cn2)cc1] | N-phenyl-2-(pyridin-3-yl)-1H- pyrrolo[3,2-c]pyridin-6-amine | 287.0 | | D | F |
| 190 | 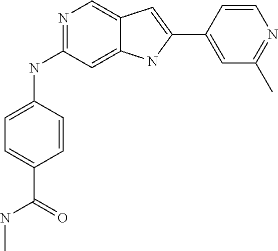 [SMILES: CNC(=O)c1ccc(Nc2cc3[nH]c(-c4ccnc(C)c4)cc3cn2)cc1] | N-methyl-4-(2-(2-methylpyridin-4- yl)-1H-pyrrolo[3,2-c]pyridin-6- ylamino)benzamide | 358.0 | | D | F |
| 191 | 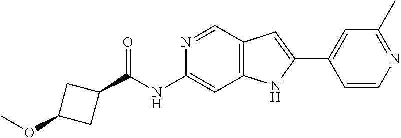 | (1s,3s)-3-methoxy-N-(2-(2- methylpyridin-4-yl)-1H-pyrrolo[3,2- c]pyridin-6- yl)cyclobutanecarboxamide | 337.0 | | D | F |
| 192 | 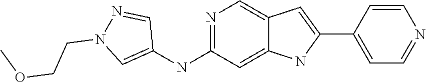 [SMILES: COCCn1cc(Nc2cc3[nH]c(-c4ccncc4)cc3cn2)cn1] | N-(1-(2-methoxyethyl)-1H- pyrazol-4-yl)-2-(pyridin-4-yl)-1H- pyrrolo[3,2-c]pyridin-6-amine | 335.5 | | E | F |
| 193 | 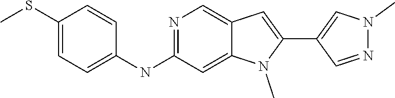 | 1-methyl-2-(1-methyl-1H-pyrazol- 4-yl)-N-(4-(methylthio)phenyl)-1H- pyrrolo[3,2-c]pyridin-6-amine | | | D | F |
| 194 | 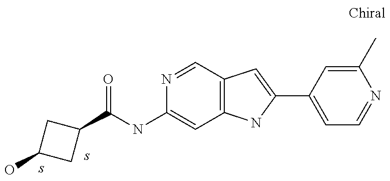 [SMILES: Cc1cc(-c2cc3cnc(NC(=O)[C@H]4C[C@@H](O)C4)cc3[nH]2)ccn1] | (1s,3s)-3-hydroxy-N-(2-(2- methypyridin-4-yl)-1H-pyrrolo[3,2- c]pyridin-6- yl)cyclobutanecarboxamide | 323.4 | | D | F |
| 195 | 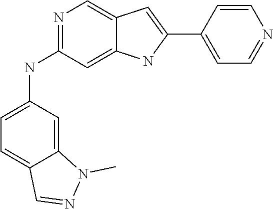 | 1-methyl-N-(2-(pyridin-4-yl)-1H- pyrrolo[3,2-c]pyridin-6-yl)-1H- indazol-6-amine | 341.2 | | F | F |
| 196 | 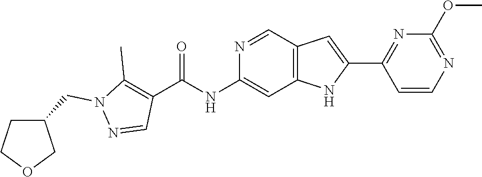 [SMILES: COc1nccc(-c2cc3cnc(NC(=O)c4cnn(C[C@H]5CCOC5)c4C)cc3[nH]2)n1] | (S)-N-(2-(2-methoxypyrimidin-4- yl)-1H-pyrrolo[3,2-c]pyridin-6-yl)- 5-methyl-1-((tetrahydrofuran-2- yl)methyl)-1H-pyrazole-4- carboxamide | 434.0 | | D | F |
| 197 | 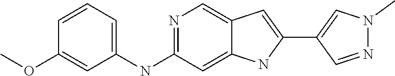 | N-(3-methoxyphenyl)-2-(1-methyl- 1H-pyrazol-4-yl)-1H-pyrrolo[3,2- c]pyridin-6-amine | 320.1 | | D | F |
| 198 | 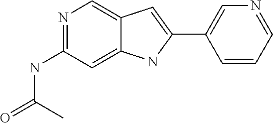 [SMILES: CC(=O)Nc1cc2[nH]c(-c3cccnc3)cc2cn1] | N-(2-(pyridin-3-yl)-1H-pyrrolo[3,2- c]pyridin-6-yl)acetamide | 253.0 | | D | F |
| 199 | 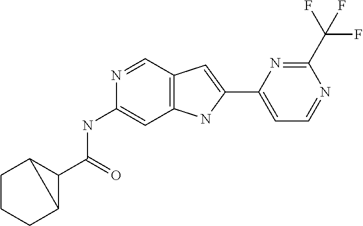 [SMILES: O=C(Nc1cc2[nH]c(-c3ccnc(C(F)(F)F)n3)cc2cn1)C1C2CCCC21] | N-(2-(2-(trifluoromethyl)pyrimidin- 4-yl)-1H-pyrrolo[3,2-c]pyridin-6- yl)bicyclo[3.1.0]hexane-6- carboxamide | 388.2 | | D | F |
| 200 | 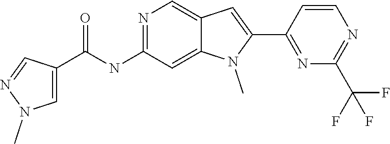 [SMILES: Cn1cc(C(=O)Nc2cc3c(cn2)cc(-c2ccnc(C(F)(F)F)n2)n3C)cn1] | 1-methyl-N-(1-methyl-2-(2- (trifluoromethyl)pyrimidin-4-yl)-1H- pyrrolo[3,2-c]pyridin-6-yl)-1H- pyrazole-4-carboxamide | 402.0 | | E | F |
| 201 | 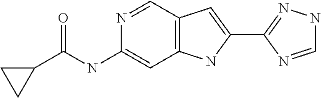 [SMILES: O=C(Nc1cc2[nH]c(-c3nc[nH]n3)cc2cn1)C1CC1] | N-(2-(1H-1,2,4-triazol-3-yl)-1H- pyrrolo[3,2-c]pyridin-6- yl)cyclopropanecarboxamide | 269.3 | | D | F |
| 202 | 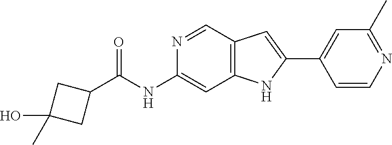 [SMILES: Cc1cc(-c2cc3cnc(NC(=O)C4CC(C)(O)C4)cc3[nH]2)ccn1] | 3-hydroxy-3-methyl-N-(2-(2- methylpyridin-4-yl)-1H-pyrrolo[3,2- c]pyridin-6- yl)cyclobutanecarboxamide | 337.0 | | D | F |
| 203 | 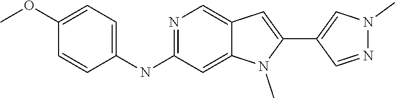 | N-(4-methoxyphenyl)-1-methyl-2- (1-methyl-1H-pyrazol-4-yl)-1H- pyrrolo[3,2-c]pyridin-6-amine | | | D | F |
| 204 | 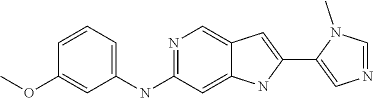 [SMILES: COc1cccc(Nc2cc3[nH]c(-c4cncn4C)cc3cn2)c1] | N-(3-methoxyphenyl)-2-(1-methyl- 1H-imidazol-5-yl)-1H-pyrrolo[3,2- c]pyridin-6-amine | | | E | F |
| 205 | 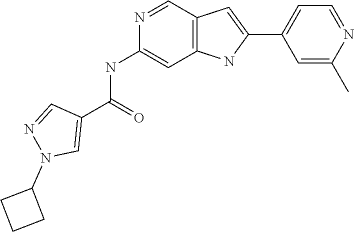 [SMILES: Cc1cc(-c2cc3cnc(NC(=O)c4cnn(C5CCC5)c4)cc3[nH]2)ccn1] | 1-cyclobutyl-N-(2-(2- methylpyridin-4-yl)-1H-pyrrolo[3,2- c]pyridin-6-yl)-1H-pyrazole-4- carboxamide | 373.0 | | D | F |
| 206 | 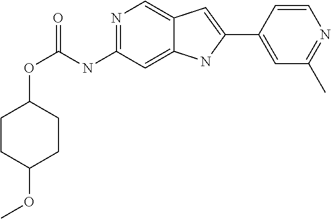 [SMILES: COC1CCC(OC(=O)Nc2cc3[nH]c(-c4ccnc(C)c4)cc3cn2)CC1] | 4-methoxycyclohexyl 2-(2- methylpyridin-4-yl)-1H-pyrrolo[3,2- c]pyridin-6-ylcarbamate | 381.3 | | D | F |
| 207 | 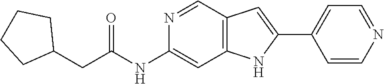 [SMILES: O=C(CC1CCCC1)Nc1cc2[nH]c(-c3ccncc3)cc2cn1] | 2-cyclopentyl-N-(2-(pyridin-4-yl)- 1H-pyrrolo[3,2-c]pyridin-6- yl)acetamide | 321.0 | | D | F |
| 208 | 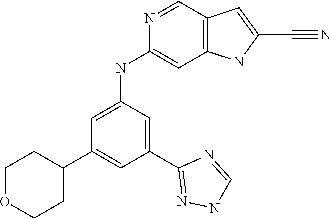 [SMILES: N#Cc1cc2cnc(Nc3cc(-c4nc[nH]n4)cc(C4CCOCC4)c3)cc2[nH]1] | 6-(3-(tetrahydro-2H-pyran-4-yl)-5- (1H-1,2,4-triazol-3- yl)phenylamino)-1H-pyrrolo[3,2- c]pyridine-2-carbonitrile | 386.0 | | D | F |
| 209 | 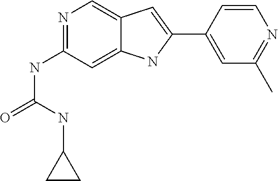 [SMILES: Cc1cc(-c2cc3cnc(NC(=O)NC4CC4)cc3[nH]2)ccn1] | 1-cyclopropyl-3-(2-(2- methylpyridin-4-yl)-1H-pyrrolo[3,2- c]pyridin-6-yl)urea | 308.0 | | D | F |
| 210 | 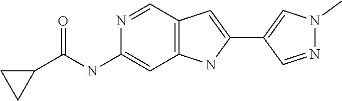 [SMILES: Cn1cc(-c2cc3cnc(NC(=O)C4CC4)cc3[nH]2)cn1] | N-(2-(1-methyl-1H-pyrazol-4-yl)- 1H-pyrrolo[3,2-c]pyridin-6- yl)cyclopropanecarboxamide | 282.2 | | D | F |
| 211 | 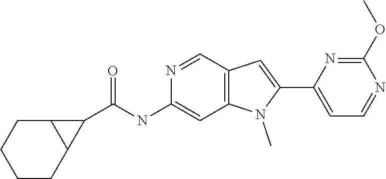 [SMILES: COc1nccc(-c2cc3cnc(NC(=O)C4C5CCCCC54)cc3n2C)n1] | N-(2-(2-methoxypyrimidin-4-yl)-1- methyl-1H-pyrrolo[3,2-c]pyridin-6- yl)bicyclo[4.1.0]heptane-7- carboxamide | 378.2 | | D | F |
| 212 | 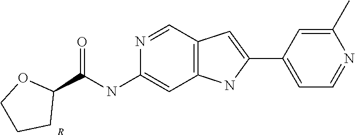 | (R)-N-(2-(2-methylpyridin-4-yl)- 1H-pyrrolo[3,2-c]pyridin-6- yl)tetrahyrdofuran-2- carboxamide | | | D | F |
| 213 | 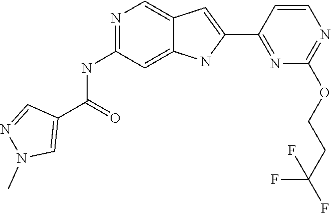 [SMILES: Cn1cc(C(=O)Nc2cc3[nH]c(-c4ccnc(OCCC(F)(F)F)n4)cc3cn2)cn1] | 1-methyl-N-(2-(2-(3,3,3- trifluoropropoxy)pyrimidin-4-yl)- 1H-pyrrolo[3,2-c]pyridin-6-yl)-1H- pyrazole-4-carboxamide | 432.1 | | D | F |
| 214 | 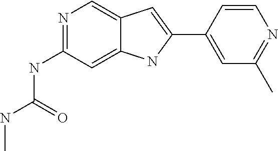 [SMILES: CNC(=O)Nc1cc2[nH]c(-c3ccnc(C)c3)cc2cn1] | 1-methyl-3-(2-(2-methylpyridin-4- yl)-1H-pyrrolo[3,2-c]pyridin-6- yl)urea | 282.0 | | D | F |
| 215 | 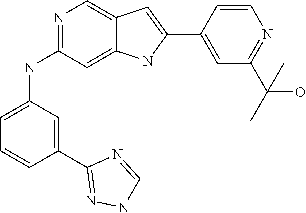 [SMILES: CC(C)(O)c1cc(-c2cc3cnc(Nc4cccc(-c5nc[nH]n5)c4)cc3[nH]2)ccn1] | 2-(4-(6-(3-(1H-1,2,4-triazol-3- yl)phenylamino)-1H-pyrrolo[3,2- c]pyridin-2-yl)pyridin-2-yl)propan- 2-ol | 412.3 | | E | F |
| 216 | 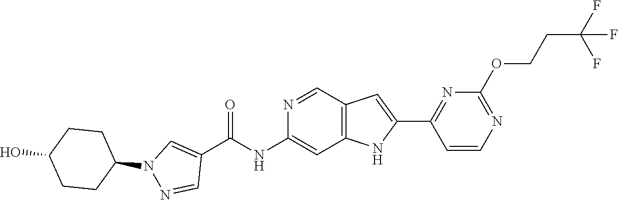 [SMILES: O=C(Nc1cc2[nH]c(-c3ccnc(OCCC(F)(F)F)n3)cc2cn1)c1cnn([C@H]2CC[C@H](O)CC2)c1] | 1-((1r,4r)-4-hydroxycyclohexyl)- N-(2-(2-(3,3,3- trifluoropropoxy)pyrimidin-4-yl)- 1H-pyrrolo[3,2-c]pyridin-6-yl)-1H- pyrazole-4-carboxamide | 516.3 | | D | F |
| 217 | 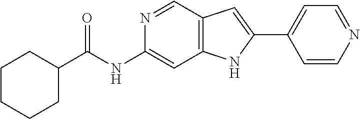 [SMILES: O=C(Nc1cc2[nH]c(-c3ccncc3)cc2cn1)C1CCCCC1] | N-(2-(pyridin-4-yl)-1H-pyrrolo[3,2- c]pyridin-6- yl)cyclohexanecarboxamide | 321.0 | | D | E |
| 218 | 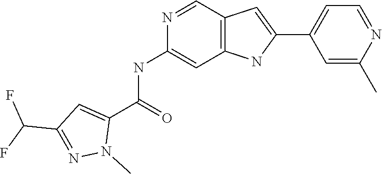 [SMILES: Cc1cc(-c2cc3cnc(NC(=O)c4cc(C(F)F)nn4C)cc3[nH]2)ccn1] | 3-(difluoromethyl)-1-methyl-N-(2- (2-methylpyridin-4-yl)-1H- pyrrolo[3,2-c]pyridin-6-yl)-1H- pyrazole-5-carboxamide | 383.0 | | D | E |
| 219 | 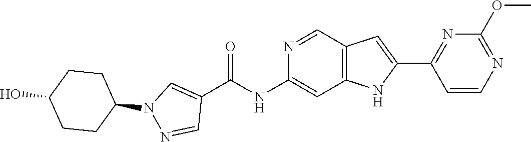 [SMILES: COc1nccc(-c2cc3cnc(NC(=O)c4cnn([C@H]5CC[C@H](O)CC5)c4)cc3[nH]2)n1] | 1-((1r,4r)-4-hydroxycyclohexyl)- N-(2-(2-methoxypyrimidin-4-yl)- 1H-pyrrolo[3,2-c]pyridin-6-yl)-1H- pyrazole-4-carboxamide | 434.0 | | D | E |
| 220 | 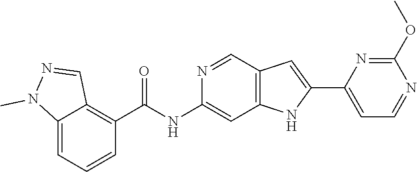 [SMILES: COc1nccc(-c2cc3cnc(NC(=O)c4cccc5c4cnn5C)cc3[nH]2)n1] | N-(2-(2-methoxypyrimidin-4-yl)- 1H-pyrrolo[3,2-c]pyridin-6-yl)-1- methyl-1H-indazole-4- carboxamide | 400.0 | | D | E |
| 221 | 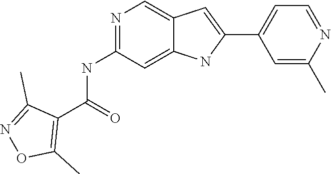 [SMILES: Cc1cc(-c2cc3cnc(NC(=O)c4c(C)noc4C)cc3[nH]2)ccn1] | 3,5-dimethyl-N-(2-(2- methylpyridin-4-yl)-1H-pyrrolo[3,2- c]pyridin-6-yl)isoxazole-4- carboxamide | | | D | E |
| 222 | 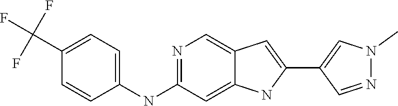 [SMILES: Cn1cc(-c2cc3cnc(Nc4ccc(C(F)(F)F)cc4)cc3[nH]2)cn1] | 2-(1-methyl-1H-pyrazol-4-yl)-N-(4- (trifluoromethyl)phenyl)-1H- pyrrolo[3,2-c]pyridin-5-amine | | | | E |
| 223 | 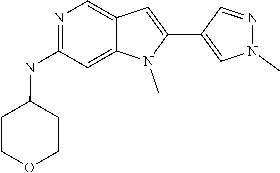 [SMILES: Cn1cc(-c2cc3cnc(NC4CCOCC4)cc3n2C)cn1] | 1-methyl-2-(1-methyl-1H-pyrazol- 4-yl)-N-(tetrahydro-2H-pyran-4- yl)-1H-pyrrolo[3,2-c]pyridin-5- amine | 312.1 | | D | E |
| 224 | 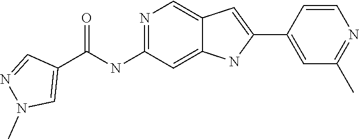 [SMILES: Cc1cc(-c2cc3cnc(NC(=O)c4cnn(C)c4)cc3[nH]2)ccn1] | 1-methyl-N-(2-(2-methylpyridin-4- yl)-1H-pyrrolo[3,2-c]pyridin-6-yl)- 1H-pyrazole-4-carboxamide | 333.0 | | D | E |
| 225 | 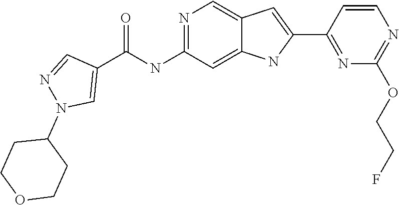 [SMILES: O=C(Nc1cc2[nH]c(-c3ccnc(OCCF)n3)cc2cn1)c1cnn(C2CCOCC2)c1] | N-(2-(2-(2-fluoroethoxy)pyrimidin- 4-yl)-1H-pyrrolo[3,2-c]pyridin-6- yl)-1-(tetrahydro-2H-pyran-4-yl)- 1H-pyrazole-4-carboxamide | 452.1 | | D | E |
| 226 | 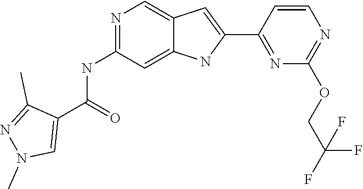 [SMILES: Cc1nn(C)cc1C(=O)Nc1cc2[nH]c(-c3ccnc(OCC(F)(F)F)n3)cc2cn1] | 1,3-dimethyl-N-(2-(2-(2,2,2- trifluoroethoxy)pyrimidin-4-yl)-1H- pyrrolo[3,2-c]pyridin-6-yl)-1H- pyrazole-4-carboxamide | 432.2 | | D | E |
| 227 | 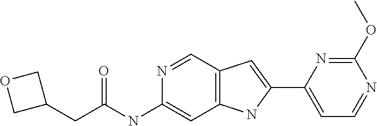 [SMILES: COc1nccc(-c2cc3cnc(NC(=O)CC4COC4)cc3[nH]2)n1] | N-(2-(2-methoxypyrimidin-4-yl)- 1H-pyrrolo[3,2-c]pyridin-6-yl)-2- (oxetan-3-yl)acetamide | 340.2 | | D | E |
| 228 | 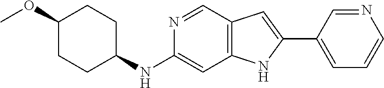 [SMILES: CO[C@H]1CC[C@@H](Nc2cc3[nH]c(-c4cccnc4)cc3cn2)CC1] | N-((1s,4s)-4-methoxycyclohexyl)- 2-(pyridin-3-yl)-1H-pyrrolo[3,2- c]pyridin-6-amine | 323.1 | | E | E |
| 229 | 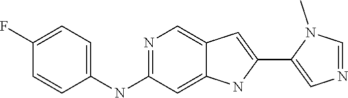 [SMILES: Cn1cncc1-c1cc2cnc(Nc3ccc(F)cc3)cc2[nH]1] | N-(4-fluorophenyl)-2-(1-methyl- 1H-imidazol-5-yl)-1H-pyrrolo[3,2- c]pyridin-6-amine | | | E | E |
| 230 | 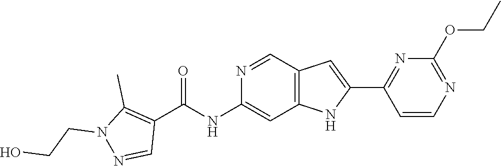 [SMILES: CCOc1nccc(-c2cc3cnc(NC(=O)c4cnn(CCO)c4C)cc3[nH]2)n1] | N-(2-(2-ethoxypyrimidin-4-yl)-1H- pyrrolo[3,2-c]pyridin-6-yl)-1-(2- hydroxyethyl)-5-methyl-1H_ pyrazole-4-carboxamide | 408.0 | | D | E |
| 231 | 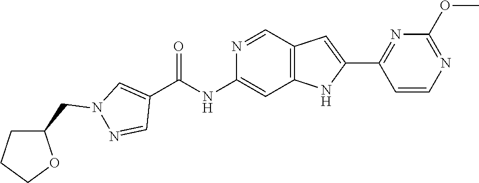 [SMILES: COc1nccc(-c2cc3cnc(NC(=O)c4cnn(C[C@@H]5CCCO5)c4)cc3[nH]2)n1] | (R)-N-(2-(2-methoxypyrimidin-4- yl)-1H-pyrrolo[3,2-c]pyridin-6-yl)- 1-((tetrahydrofuran-2-yl)methyl)- 1H-pyrazole-4-carboxamide | 420.0 | | E | E |
| 232 | 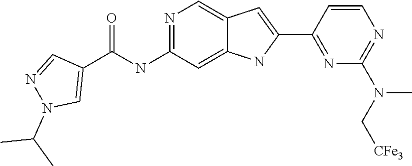 | 1-isopropyl-N-(2-(2-(methyl(2,2,2- trifluoroethyl)amino)pyrimidin-4- yl)-1H-pyrrolo[3,2-c]pyridin-6-yl)- 1H-pyrazole-4-carboxamide | 459.2 | | D | E |
| 233 | 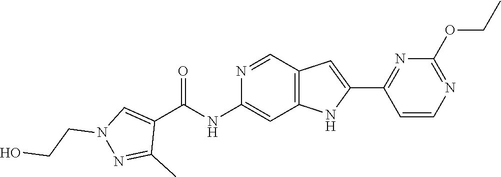 [SMILES: CCOc1nccc(-c2cc3cnc(NC(=O)c4cn(CCO)nc4C)cc3[nH]2)n1] | N-(2-(2-ethoxypyrimidin-4-yl)-1H- pyrrolo[3,2-c]pyridin-6-yl)-1-(2- hydroxyethyl)-3-methyl-1H- pyrazole-4-carboxamide | 408.0 | | D | E |
| 234 | 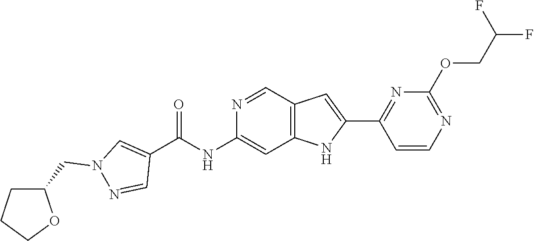 [SMILES: O=C(Nc1cc2[nH]c(-c3ccnc(OCC(F)F)n3)cc2cn1)c1cnn(C[C@H]2CCCO2)c1] | (S)-N-(2-(2-(2,2- difluoroethoxy)pyrimidin-4-yl)-1H- pyrrolo[3,2-c]pyridin-6-yl)-1- ((tetrahydrofuran-2-yl)methyl)-1H- pyrazole-4-carboxamdie | 470.1 | | E | E |
| 235 | 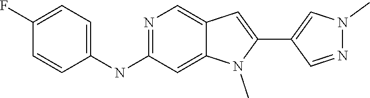 [SMILES: Cn1cc(-c2cc3cnc(Nc4ccc(F)cc4)cc3n2C)cn1] | N-(4-fluorophenyl)-1-methyl-2-(1- methyl-1H-pyrazol-4-yl)-1H- pyrrolo[3,2-c]pyridin-6-amine | | | D | E |
| 236 | 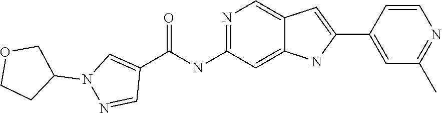 [SMILES: Cc1cc(-c2cc3cnc(NC(=O)c4cnn(C5CCOC5)c4)cc3[nH]2)ccn1] | N-(2-(2-methylpyridin-4-yl)-1H- pyrrolo[3,2-c]pyridin-6-yl)-1- (tetrahydrofuran-3-yl)-1H- pyrazole-4-carboxamide | 389.0 | | E | E |
| 237 | 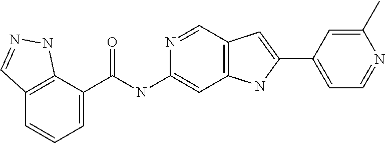 [SMILES: Cc1cc(-c2cc3cnc(NC(=O)c4cccc5cn[nH]c45)cc3[nH]2)ccn1] | N-(2-(2-methylpyridin-4-yl)-1H- pyrrolo[3,2-c]pyridin-6-yl)-1H- indazole-7-carboxamide | | | D | E |
| 238 | 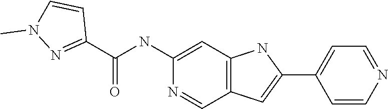 [SMILES: Cn1ccc(C(=O)Nc2cc3[nH]c(-c4ccncc4)cc3cn2)n1] | 1-methyl-N-(2-(pyridin-4-yl)-1H- pyrrolo[3,2-c]pyridin-6-yl)-1H- pyrazole-3-carboxamide | 318.1 | | D | D |
| 239 | 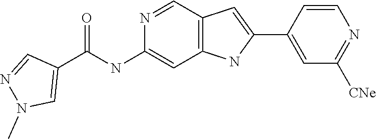 | N-(2-(2-cyanopyridin-4-yl)-1H- pyrrolo[3,2-c]pyridin-6-yl)-1- methyl-1H-pyrazole-4- carboxamide | 344.3 | | D | E |
| 240 | 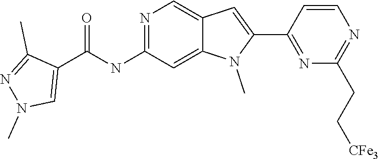 | 1,3-dimethyl-N-(1-methyl-2-(2- (2,2,2- trifluoroethylamino)pyrimidin-4-yl)- 1H-pyrrolo[3,2-c]pyridin-6-yl)-1H- pyrazole-4-carboxamide | 445.0 | | D | D |
| 241 | 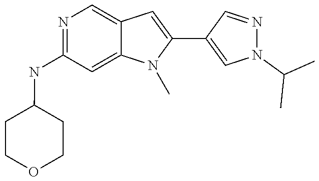 [SMILES: CC(C)n1cc(-c2cc3cnc(NC4CCOCC4)cc3n2C)cn1] | 2-(1-isopropyl-1H-pyrazol-4-yl)-1- methyl-N-(tetrahydro-2H-pyran-4- yl)-1H-pyrrolo[3,2-c]pyridin-6- amine | 340.2 | | E | D |
| 242 | 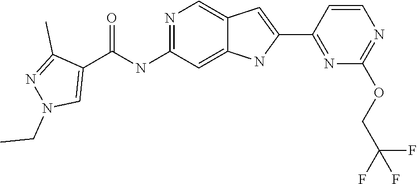 [SMILES: CCn1cc(C(=O)Nc2cc3[nH]c(-c4ccnc(OCC(F)(F)F)n4)cc3cn2)c(C)n1] | 1-ethyl-3-methyl-N-(2-(2-(2,2,2- trifluoroethoxy)pyrimidin-4-yl)-1H- pyrrolo[3,2-c]pyridin-6-yl)-1H- pyrazole-4-carboxamide | 446.0 | | D | D |
| 243 | 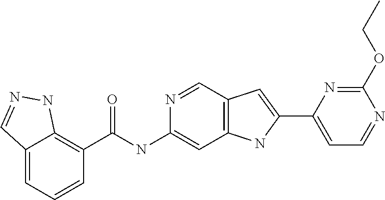 [SMILES: CCOc1nccc(-c2cc3cnc(NC(=O)c4cccc5cn[nH]c45)cc3[nH]2)n1] | N-(2-(2-ethoxypyrimidin-4-yl)-1H- pyrrolo[3,2-c]pyridin-6-yl)-1H- indazole-7-carboxamide | 400.0 | | D | D |
| 244 | 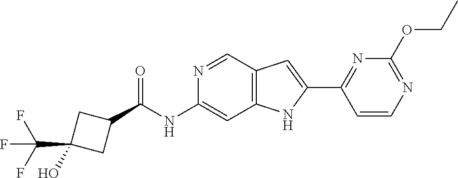 | (1r,3r)-N-(2-(2-ethoxypyrimidin-4- yl)-1H-pyrrolo[3,2-c]pyridin-6-yl)- 3-hydroxy-3- (trifluoromethyl)cyclobutanecarbox- amide | | | | |
| 245 | 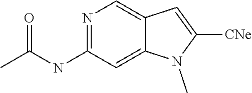 | N-(2-cyano-1-methyl-1H- pyrrolo[3,2-c]pyridin-6- yl)acetamide | 215.0 | | D | D |
| 246 | 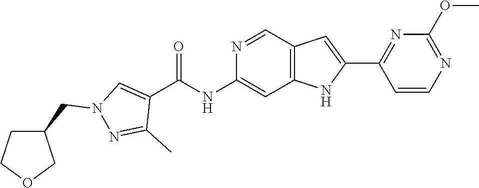 [SMILES: COc1nccc(-c2cc3cnc(NC(=O)c4cn(C[C@@H]5CCOC5)nc4C)cc3[nH]2)n1] | (R)-N-(2-(2-methoxypyrimidin-4- yl)-1H-pyrrolo[3,2-c]pyridin-6-yl)- 3-methyl-1-((tetrahydrofuran-3- yl)methyl)-1H-pyrazole-4- carboxamide | 434.0 | | D | D |
| 247 | 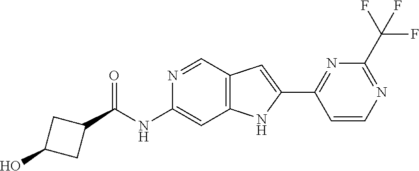 | (1s,3s)-3-hydroxy-N-(2-(2- (trifluoromethyl)pyrimidin-4-yl)-1H- pyrrolo[3,2-c]pyridin-6- yl)cyclobutanecarboxamide | | | E | D |
| 248 | 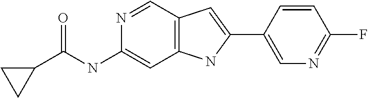 [SMILES: O=C(Nc1cc2[nH]c(-c3ccc(F)nc3)cc2cn1)C1CC1] | N-(2-(6-fluoropyridin-3-yl)-1H- pyrrolo[3,2-c]pyridin-6- yl)cyclopropanecarboxamide | 297.3 | | D | D |
| 249 | 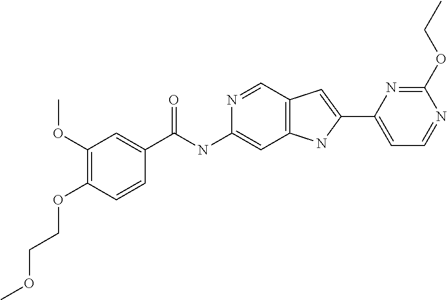 [SMILES: CCOc1nccc(-c2cc3cnc(NC(=O)c4ccc(OCCOC)c(OC)c4)cc3[nH]2)n1] | N-(2-(2-ethoxypyrimidin-4-yl)-1H- pyrrolo[3,2-c]pyridin-6-yl)-3- methoxy-4-(2- methoxyethoxy)benzamide | 464.2 | | D | D |
| 250 | 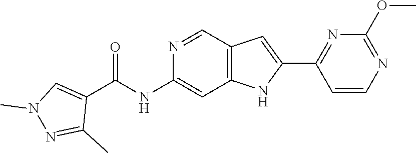 | N-(2-(2-methoxypyrimidin-4-yl)- 1H-pyrrolo[3,2-c]pyridin-6-yl)-1,3- dimethyl-1H-pyrazole-4- carboxamide | 363.0 | | D | D |
| 251 | 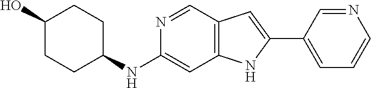 [SMILES: O[C@H]1CC[C@@H](Nc2cc3[nH]c(-c4cccnc4)cc3cn2)CC1] | (1s,4s)-4-(2-(pyridin-3-yl)-1H- pyrrolo[3,2-c]pyridin-6- ylamino)cyclohexanol | 309.1 | | D | D |
| 252 | 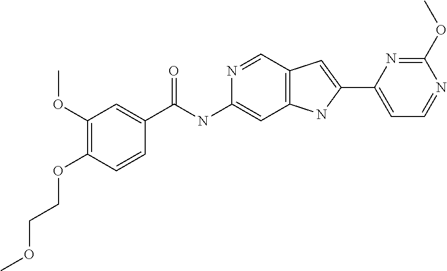 [SMILES: COCCOc1ccc(C(=O)Nc2cc3[nH]c(-c4ccnc(OC)n4)cc3cn2)cc1OC] | 3-methoxy-4-(2-methoxyethoxy)- N-(2-(2-methoxypyrimidin-4-yl)- 1H-pyrrolo[3,2-c]pyridin-6- yl)benzamide | 450.0 | | D | D |
| 253 | 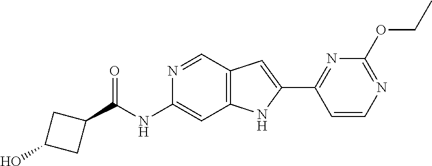 [SMILES: CCOc1nccc(-c2cc3cnc(NC(=O)[C@H]4C[C@H](O)C4)cc3[nH]2)n1] | (1r,3r)-N-(2-(2-ethoxypyrimidin-4- yl)-1H-pyrrolo[3,2-c]pyridin-6-yl)- 3- hydroxycyclobutanecarboxamide | 354.2 | | D | D |
| 254 | 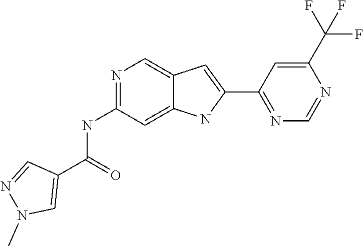 [SMILES: Cn1cc(C(=O)Nc2cc3[nH]c(-c4cc(C(F)(F)F)ncn4)cc3cn2)cn1] | 1-methyl-N-(2-(6- (trifluoromethyl)pyrimidin-4-yl)-1H- pyrrolo[3,2-c]pyridin-6-yl)-1H- pyrazole-4-carboxamide | 388.0 | | D | D |
| 255 | 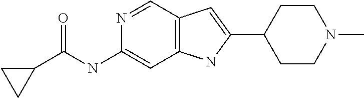 [SMILES: CN1CCC(c2cc3cnc(NC(=O)C4CC4)cc3[nH]2)CC1] | N-(2-(1-methylpiperidin-4-yl)-1H- pyrrolo[3,2-c]pyridin-6- yl)cyclopropanecarboxamide | 299.2 | | D | D |
| 256 | 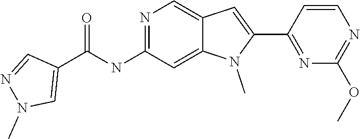 [SMILES: COc1nccc(-c2cc3cnc(NC(=O)c4cnn(C)c4)cc3n2C)n1] | N-(2-(2-methoxypyrimidin-4-yl)-1- methyl-1H-pyrrolo[3,2-c]pyridin-6- yl)-1-methyl-1H-pyrazole-4- carboxamide | 364.0 | | D | D |
| 257 | 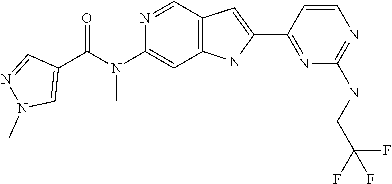 [SMILES: CN(C(=O)c1cnn(C)c1)c1cc2[nH]c(-c3ccnc(NCC(F)(F)F)n3)cc2cn1] | N,1-dimethyl-N-(2-(2-(2,2,2- trifluoroethylamino)pyrimidin-4-yl)- 1H-pyrrolo[3,2-c]pyridin-6-yl)-1H- pyrazole-4-carboxamide | 431.3 | | D | D |
| 258 | 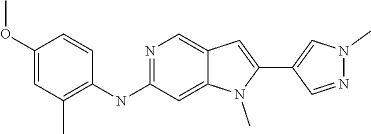 [SMILES: COc1ccc(Nc2cc3c(cn2)cc(-c2cnn(C)c2)n3C)c(C)c1] | N-(4-methoxy-2-methylphenyl)-1- methyl-2-(1-methyl-1H-pyrazol-4- yl)-1H-pyrrolo[3,2-c]pyridin-6- amine | | | D | D |
| 259 | 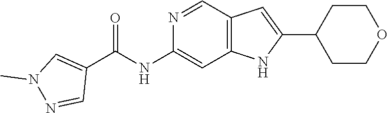 [SMILES: Cn1cc(C(=O)Nc2cc3[nH]c(C4CCOCC4)cc3cn2)cn1] | 1-methyl-N-(2-(tetrahydro-2H- pyran-4-yl)-1H-pyrrolo[3,2- c]pyridin-6-yl)-1H-pyrazole-4- carboxamide | 326.0 | | D | D |
| 260 | 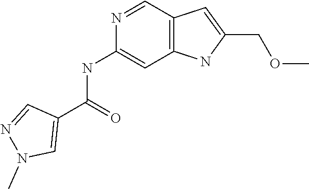 [SMILES: COCc1cc2cnc(NC(=O)c3cnn(C)c3)cc2[nH]1] | N-(2-(methoxymethyl)-1H- pyrrolo[3,2-c]pyridin-6-yl)-1- methyl-1H-pyrazole-4- carboxamide | 286.0 | | D | D |
| 261 | 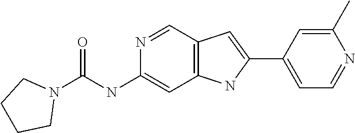 [SMILES: Cc1cc(-c2cc3cnc(NC(=O)N4CCCC4)cc3[nH]2)ccn1] | N-(2-(2-methylpyridin-4-yl)-1H- pyrrolo[3,2-c]pyridin-6- yl)pyrrolidine-1-carboxamide | | | D | D |
| 262 | 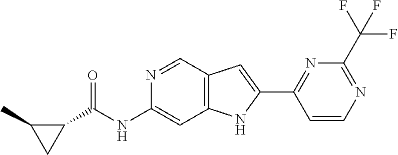 [SMILES: C[C@@H]1C[C@H]1C(=O)Nc1cc2[nH]c(-c3ccnc(C(F)(F)F)n3)cc2cn1] | (1S,2S)-2-methyl-N-(2-(2- (trifluoromethyl)pyrimidin-4-yl)-1H- pyrrolo[3,2-c]pyridin-6- yl)cyclopropanecarboxamide | | | D | D |
| 263 | 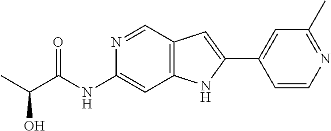 [SMILES: Cc1cc(-c2cc3cnc(NC(=O)[C@H](C)O)cc3[nH]2)ccn1] | (R)-2-hydroxy-N-(2-(2- methylpyridin-4-yl)-1H-pyrrolo[3,2- c]pyridin-6-yl)propanamide | | | D | D |
| 264 | 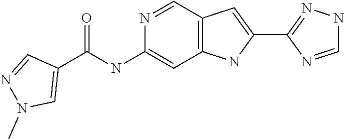 [SMILES: Cn1cc(C(=O)Nc2cc3[nH]c(-c4nc[nH]n4)cc3cn2)cn1] | N-(2-(1H-1,2,4-triazol-3-yl)-1H- pyrrolo[3,2-c]pyridin-6-yl)-1- methyl-1H-pyrazole-4- carboxamide | 309.2 | | D | D |
| 265 | 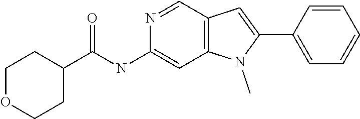 [SMILES: Cn1c(-c2ccccc2)cc2cnc(NC(=O)C3CCOCC3)cc21] | N-(1-methyl-2-phenyl-1H- pyrrolo[3,2-c]pyridin-6- yl)tetrahydro-2H-pyran-4- carboxamide | 336.1 | | D | D |
| 266 | 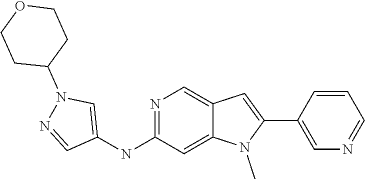 [SMILES: Cn1c(-c2cccnc2)cc2cnc(Nc3cnn(C4CCOCC4)c3)cc21] | 1-methyl-2-(pyridin-3-yl)-N-(1- (tetrahydro-2H-pyran-4-yl)-1H- pyrazol-4-yl)-1H-pyrrolo[3,2- c]pyridin-6-amine | 375.2 | | D | D |
| 267 | 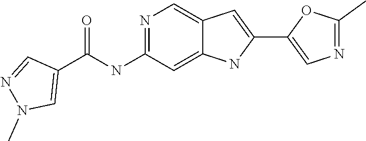 [SMILES: Cc1ncc(-c2cc3cnc(NC(=O)c4cnn(C)c4)cc3[nH]2)o1] | 1-methyl-N-(2-(2-methyloxazol-5- yl)-1H-pyrrolo[3,2-c]pyridin-6-yl)- 1H-pyrazole-4-carboxamide | 323.3 | | D | D |
| 268 | 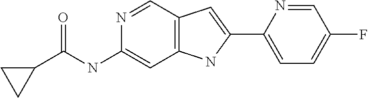 [SMILES: O=C(Nc1cc2[nH]c(-c3ccc(F)cn3)cc2cn1)C1CC1] | N-(2-(5-fluoropyridin-2-yl)-1H- pyrrolo[3,2-c]pyridin-6- yl)cyclopropanecarboxamide | 297.1 | | D | D |
| 269 | 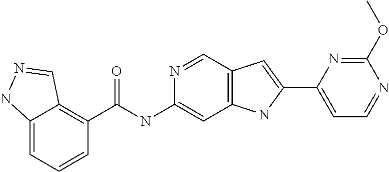 [SMILES: COc1nccc(-c2cc3cnc(NC(=O)c4cccc5[nH]ncc45)cc3[nH]2)n1] | N-(2-(2-methoxypyrimidin-4-yl)- 1H-pyrrolo[3,2-c]pyridin-6-yl)-1H- indazole-4-carboxamide | | | D | D |
| 270 | 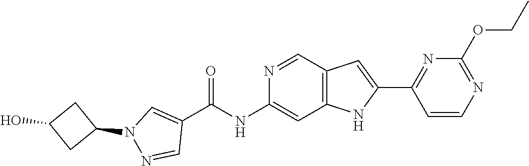 [SMILES: CCOc1nccc(-c2cc3cnc(NC(=O)c4cnn([C@H]5C[C@H](O)C5)c4)cc3[nH]2)n1] | N-(2-(2-ethoxypyrimidin-4-yl)-1H- pyrrolo[3,2-c]pyridin-6-yl)-1- ((1r,3r)-3-hydroxycyclobutyl)-1H- pyrazole-4-carboxamide | 420.0 | | D | D |
| 271 | 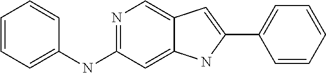 [SMILES: c1ccc(Nc2cc3[nH]c(-c4ccccc4)cc3cn2)cc1] | N,2-diphenyl-1H-pyrrolo[3,2- c]pyridin-6-amine | | | D | D |
| 272 | 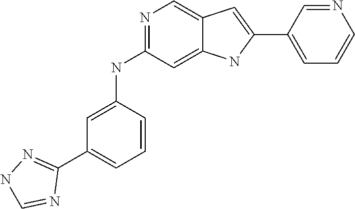 [SMILES: c1cc(Nc2cc3[nH]c(-c4cccnc4)cc3cn2)cc(-c2nc[nH]n2)c1] | N-(3-(1H-1,2,4-triazol-3- yl)phenyl)-2-(pyridin-3-yl)-1H- pyrrolo[3,2-c]pyridin-6-amine | 354.0 | | D | D |
| 273 | 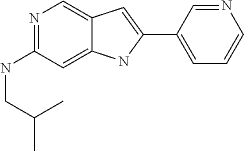 [SMILES: CC(C)CNc1cc2[nH]c(-c3cccnc3)cc2cn1] | N-isobutyl-2-(pyridin-3-yl)-1H- pyrrolo[3,2-c]pyridin-6-amine | 267.0 | | D | D |
| 274 | 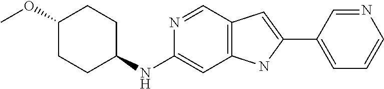 [SMILES: CO[C@H]1CC[C@H](Nc2cc3[nH]c(-c4cccnc4)cc3cn2)CC1] | N-((1r,4r)-4-methoxycyclohexyl)- 2-(pyridin-3-yl)-1H-pyrrolo[3,2- c]pyridin-6-amine | 323.4 | | D | D |
| 275 | 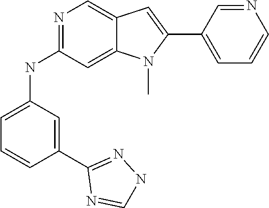 [SMILES: Cn1c(-c2cccnc2)cc2cnc(Nc3cccc(-c4nc[nH]n4)c3)cc21] | N-(3-(1H-1,2,4-triazol-3- yl)phenyl)-1-methyl-2-(pyridin-3- yl)-1H-pyrrolo[3,2-c]pyridin-6- amine | 368.1 | | D | D |
| 276 | 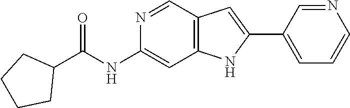 [SMILES: O=C(Nc1cc2[nH]c(-c3cccnc3)cc2cn1)C1CCCC1] | N-(2-(pyridin-3-yl)-1H-pyrrolo[3,2- c]pyridin-6- yl)cyclopentanecarboxamide | 307.0 | | D | D |
| 277 | 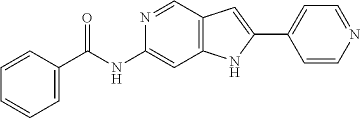 [SMILES: O=C(Nc1cc2[nH]c(-c3ccncc3)cc2cn1)c1ccccc1] | N-(2-(pyridin-4-yl)-1H-pyrrolo[3,2- c]pyridin-6-yl)benzamide | 315.0 | | D | D |
| 278 | 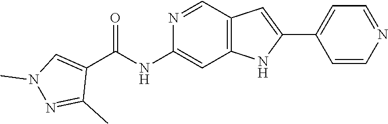 [SMILES: Cc1nn(C)cc1C(=O)Nc1cc2[nH]c(-c3ccncc3)cc2cn1] | 1,3-dimethyl-N-(2-(pyridin-4-yl)- 1H-pyrrolo[3,2-c]pyridin-6-yl)-1H- pyrazole-4-carboxamide | 333.0 | | D | D |
| 279 | 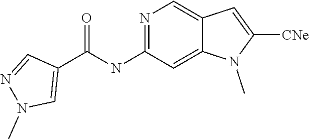 | N-(2-cyano-1-methyl-1H- pyrrolo[3,2-c]pyridin-6-yl)-1- methyl-1H-pyrazole-4- carboxamide | 280.9 | | D | D |
| 280 | 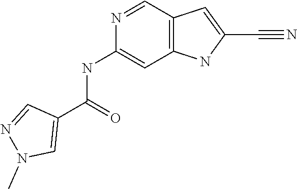 [SMILES: Cn1cc(C(=O)Nc2cc3[nH]c(C#N)cc3cn2)cn1] | N-(2-cyano-1H-pyrrolo[3,2- c]pyridin-6-yl)-1-methyl-1H- pyrazole-4-carboxamide | 267.0 | | D | D |
| 281 | 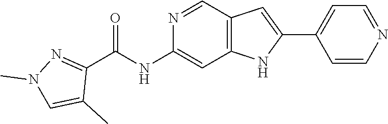 [SMILES: Cc1cn(C)nc1C(=O)Nc1cc2[nH]c(-c3ccncc3)cc2cn1] | 1,4-dimethyl-N-(2-(pyridin-4-yl)- 1H-pyrrolo[3,2-c]pyridin-6-yl)-1H- pyrazole-3-carboxamide | 333.0 | | D | D |
| 282 | 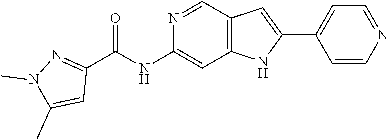 [SMILES: Cc1cc(C(=O)Nc2cc3[nH]c(-c4ccncc4)cc3cn2)nn1C] | 1,5-dimethyl-N-(2-(pyridin-4-yl)- 1H-pyrrolo[3,2-c]pyridin-6-yl)-1H- pyrazole-3-carboxamide | 333.0 | | D | D |
| 283 | 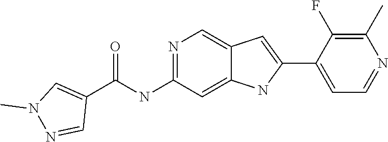 | N-(2-(3-fluoro-2-methylpyridin-4- yl)-1H-pyrrolo[3,2-c]pyridin-6-yl)- 1-methyl-1H-pyrazole-4- carboxamide | 351.0 | | D | D |
| 284 | 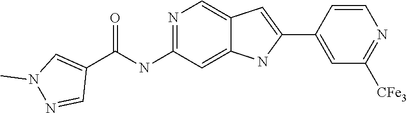 | 1-methyl-N-(2-(2- (trifluoromethyl)pyridin-4-yl)-1H- pyrrolo[3,2-c]pyridin-6-yl)-1H- pyrazole-4-carboxamide | 386.7 | | D | D |
| 285 | 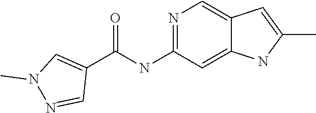 | 1-methyl-N-(2-methyl-1H- pyrrolo[3,2-c]pyridin-6-yl)-1H- pyrazole-4-carboxamide | 256.0 | | D | D |
| 286 | 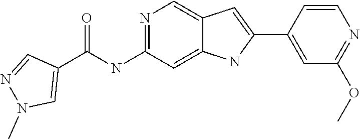 [SMILES: COc1cc(-c2cc3cnc(NC(=O)c4cnn(C)c4)cc3[nH]2)ccn1] | N-(2-(2-methoxypyridin-4-yl)-1H- pyrrolo[3,2-c]pyridin-6-yl)-1- methyl-1H-pyrazole-4- carboxamide | 348.9 | | D | D |
| 287 | 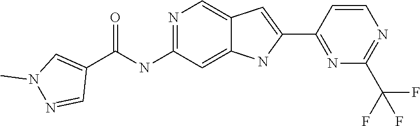 [SMILES: Cn1cc(C(=O)Nc2cc3[nH]c(-c4ccnc(C(F)(F)F)n4)cc3cn2)cn1] | 1-methyl-N-(2-(2- (trifluoromethyl)pyrimidin-4-yl)-1H- pyrrolo[3,2-c]pyridin-6-yl)-1H- pyrazole-4-carboxamide | 388.0 | | D | D |
| 288 | 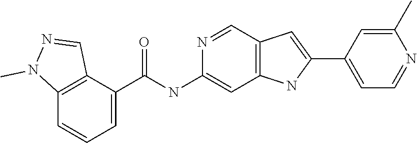 | 1-methyl-N-(2-(2-methylpyridin-4- yl)-1H-pyrrolo[3,2-c]pyridin-6-yl)- 1H-indazole-4-carboxamide | 383.3 | | D | D |
| 289 | 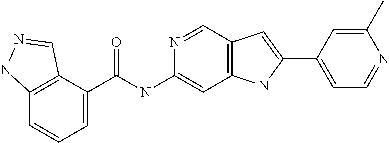 [SMILES: Cc1cc(-c2cc3cnc(NC(=O)c4cccc5[nH]ncc45)cc3[nH]2)ccn1] | N-(2-(2-methylpyridin-4-yl)-1H- pyrrolo[3,2-c]pyridin-6-yl)-1H- indazole-4-carboxamide | | | D | D |
| 290 | 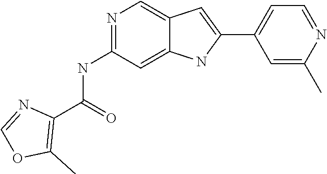 [SMILES: Cc1cc(-c2cc3cnc(NC(=O)c4ncoc4C)cc3[nH]2)ccn1] | 5-methyl-N-(2-(2-methylpyridin-4- yl)-1H-pyrrolo[3,2-c]pyridin-6- yl)oxazole-4-carboxamide | 334.3 | | D | D |
| 291 | 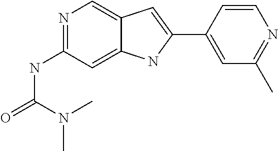 [SMILES: Cc1cc(-c2cc3cnc(NC(=O)N(C)C)cc3[nH]2)ccn1] | 1,1-dimethyl-3-(2-(2-methylpyridin- 4-yl)-1H-pyrrolo[3,2-c]pyridin-6- yl)urea | 296.0 | | D | D |
| 292 | 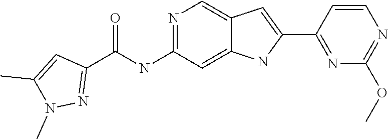 [SMILES: COc1nccc(-c2cc3cnc(NC(=O)c4cc(C)n(C)n4)cc3[nH]2)n1] | N-(2-(2-methoxypyrimidin-4-yl)- 1H-pyrrolo[3,2-c]pyridin-6-yl)-1,5- dimethyl-1H-pyrazole-3- carboxamide | 364.0 | | D | D |
| 293 | 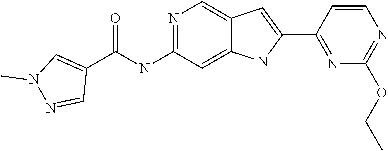 [SMILES: CCOc1nccc(-c2cc3cnc(NC(=O)c4cnn(C)c4)cc3[nH]2)n1] | N-(2-(2-ethoxypyrimidin-4-yl)-1H- pyrrolo[3,2-c]pyridin-6-yl)-1- methyl-1H-pyrazole-4- carboxamide | 364.1 | | D | D |
| 294 | 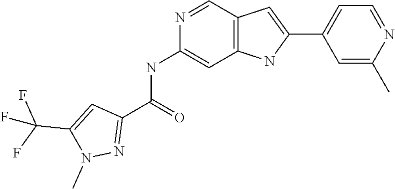 [SMILES: Cc1cc(-c2cc3cnc(NC(=O)c4cc(C(F)(F)F)n(C)n4)cc3[nH]2)ccn1] | 1-methyl-N-(2-(2-methylpyridin-4- yl)-1H-pyrrolo[3,2-c]pyridin-6-yl)- 5-(trifluoromethyl)-1H-pyrazole-3- carboxamide | 401.0 | | D | D |
| 295 | 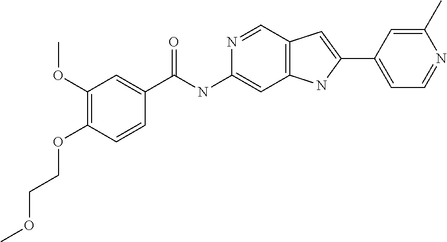 | 3-methoxy-4-(2-methoxyethoxy)- N-(2-(2-methylpyridin-4-yl)-1H- pyrrolo[3,2-c]pyridin-6- yl)benzamide | | | D | D |
| 296 | 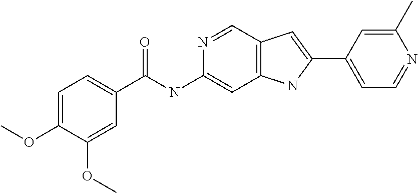 | 3,4-dimethoxy-N-(2-(2- methylpyridin-4-yl)-1H-pyrrolo[3,2- c]pyridin-6-yl)benzamide | 389.1 | | D | D |
| 297 | 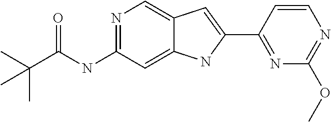 [SMILES: COc1nccc(-c2cc3cnc(NC(=O)C(C)(C)C)cc3[nH]2)n1] | N-(2-(2-methoxypyrimidin-4-yl)- 1H-pyrrolo[3,2-c]pyridin-6- yl)pivalamide | 326.3 | | D | D |
| 298 | 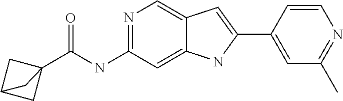 [SMILES: Cc1cc(-c2cc3cnc(NC(=O)C45CC(C4)C5)cc3[nH]2)ccn1] | N-(2-(2-methylpyridin-4-yl)-1H- pyrrolo[3,2-c]pyridin-6- yl)bicyclo[1.1.1]pentane-1- carboxamide | 319.0 | | D | D |
| 299 | 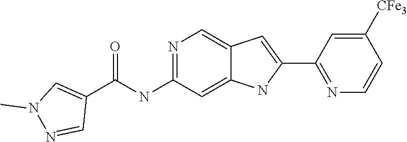 | 1-methyl-N-(2-(4- (trifluoromethyl)pyridin-2-yl)-1H- pyrrolo[3,2-c]pyridin-6-yl)-1H- pyrazole-4-carboxamide | 387.0 | | D | D |
| 300 | 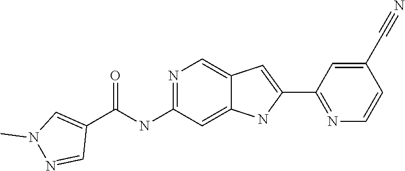 [SMILES: Cn1cc(C(=O)Nc2cc3[nH]c(-c4cc(C#N)ccn4)cc3cn2)cn1] | N-(2-(4-cyanopyridin-2-yl)-1H- pyrrolo[3,2-c]pyridin-6-yl)-1- methyl-1H-pyrazole-4- carboxamide | 344.1 | | D | D |
| 301 | 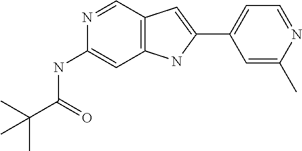 [SMILES: Cc1cc(-c2cc3cnc(NC(=O)C(C)(C)C)cc3[nH]2)ccn1] | N-(2-(2-methylpyridin-4-yl)-1H- pyrrolo[3,2-c]pyridin-6- yl)pivalamide | 309.0 | | D | D |
| 302 | 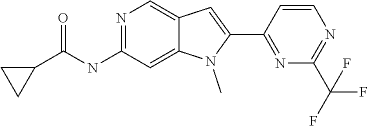 [SMILES: Cn1c(-c2ccnc(C(F)(F)F)n2)cc2cnc(NC(=O)C3CC3)cc21] | N-(1-methyl-2-(2- (trifluoromethyl)pyrimidin-4-yl)-1H- pyrrolo[3,2-c]pyridin-6- yl)cyclopropanecarboxamide | 362.2 | | D | D |
| 303 | 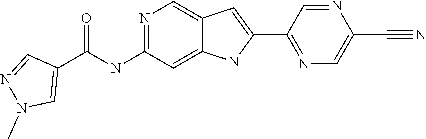 [SMILES: Cn1cc(C(=O)Nc2cc3[nH]c(-c4cnc(C#N)cn4)cc3cn2)cn1] | N-(2-(5-cyanopyrazin-2-yl)-1H- pyrrolo[3,2-c]pyridin-6-yl)-1- methyl-1H-pyrazole-4- carboxamide | 345.0 | | D | D |
| 304 | 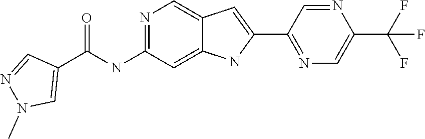 [SMILES: Cn1cc(C(=O)Nc2cc3[nH]c(-c4cnc(C(F)(F)F)cn4)cc3cn2)cn1] | 1-methyl-N-(2-(5- (trifluoromethyl)pyrazin-2-yl)-1H- pyrrolo[3,2-c]pyridin-6-yl)-1H- pyrazole-4-carboxamide | 388.0 | | E | D |
| 305 | 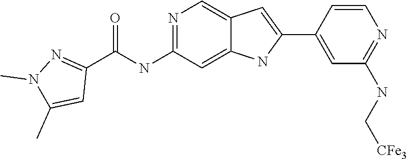 | 1,5-dimethyl-N-(2-(2-(2,2,2- trifluoroethylamino)pyridin-4-yl)- 1H-pyrrolo[3,2-c]pyridin-6-yl)-1H- pyrazole-3-carboxamide | 430.1 | | D | D |
| 306 | 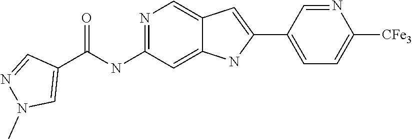 | 1-methyl-N-(2-(6- (trifluoromethyl)pyridin-3-yl)-1H- pyrrolo[3,2-c]pyridin-6-yl)-1H- pyrazole-4-carboxamide | 387.2 | | D | D |
| 307 | 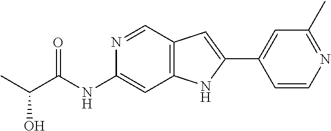 [SMILES: Cc1cc(-c2cc3cnc(NC(=O)[C@@H](C)O)cc3[nH]2)ccn1] | (S)-2-hydroxy-N-(2-(2- methylpyridin-4-yl)-1H-pyrrolo[3,2- c]pyridin-6-yl)propanamide | | | D | D |
| 308 | 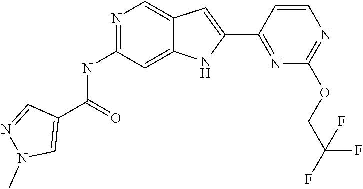 [SMILES: Cn1cc(C(=O)Nc2cc3[nH]c(-c4ccnc(OCC(F)(F)F)n4)cc3cn2)cn1] | 1-methyl-N-(2-(2-(2,2,2- trifluoroethoxy)pyrimidin-4-yl)-1H- pyrrolo[3,2-c]pyridin-6-yl)-1H- pyrazole-4-carboxamide | 418.1 | | D | D |
| 309 | 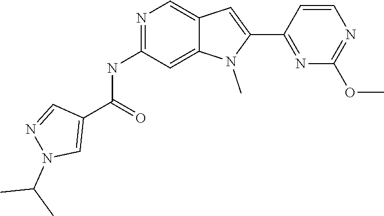 [SMILES: COc1nccc(-c2cc3cnc(NC(=O)c4cnn(C(C)C)c4)cc3n2C)n1] | 1-isopropyl-N-(2-(2- methoxypyrimidin-4-yl)-1-methyl- 1H-pyrrolo[3,2-c]pyridin-6-yl)-1H- pyrazole-4-carboxamide | 392.1 | | D | D |
| 310 | 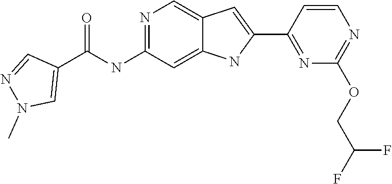 [SMILES: Cn1cc(C(=O)Nc2cc3[nH]c(-c4ccnc(OCC(F)F)n4)cc3cn2)cn1] | N-(2-(2-(2,2- difluoroethoxy)pyrimidin-4-yl)-1H- pyrrolo[3,2-c]pyridin-6-yl)-1- methyl-1H-pyrazole-4- carboxamide | 400.1 | | D | D |
| 311 | 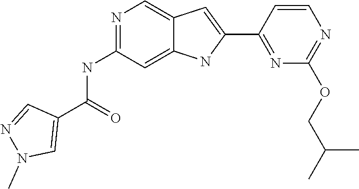 [SMILES: CC(C)COc1nccc(-c2cc3cnc(NC(=O)c4cnn(C)c4)cc3[nH]2)n1] | N-(2-(2-isobutoxypyrimidin-4-yl)- 1H-pyrrolo[3,2-c]pyridin-6-yl)-1- methyl-1H-pyrazole-4- carboxamide | 392.1 | | D | D |
| 312 | 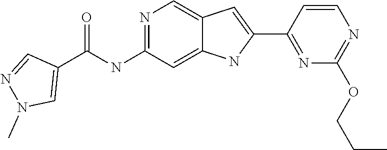 [SMILES: CCCOc1nccc(-c2cc3cnc(NC(=O)c4cnn(C)c4)cc3[nH]2)n1] | 1-methyl-N-(2-(2- propoxypyrimidin-4-yl)-1H- pyrrolo[3,2-c]pyridin-6-yl)-1H- pyrazole-4-carboxamide | 378.2 | | D | D |
| 313 | 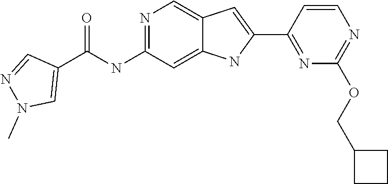 [SMILES: Cn1cc(C(=O)Nc2cc3[nH]c(-c4ccnc(OCC5CCC5)n4)cc3cn2)cn1] | N-(2-(2- (cyclobutylmethoxy)pyrimidin-4- yl)-1H-pyrrolo[3,2-c]pyridin-6-yl)- 1-methyl-1H-pyrazole-4- carboxamide | 404.1 | | D | D |
| 314 | 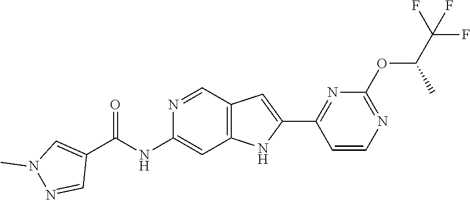 [SMILES: C[C@H](Oc1nccc(-c2cc3cnc(NC(=O)c4cnn(C)c4)cc3[nH]2)n1)C(F)(F)F] | (R)-1-methyl-N-(2-(2-(1,1,1- trifluoropropan-2-yloxy)pyrimidin- 4-yl)-1H-pyrrolo[3,2-c]pyridin-6- yl)-1H-pyrazole-4-carboxamide | 432.1 | | D | D |
| 315 | 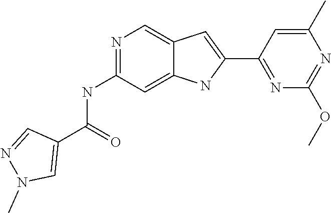 [SMILES: COc1nc(C)cc(-c2cc3cnc(NC(=O)c4cnn(C)c4)cc3[nH]2)n1] | N-(2-(2-methoxy-6- methylpyrimidin-4-yl)-1H- pyrrolo[3,2-c]pyridin-6-yl)-1- methyl-1H-pyrazole-4- carboxamide | 364.2 | | D | D |
| 316 | 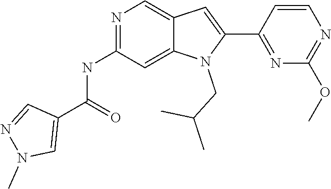 [SMILES: COc1nccc(-c2cc3cnc(NC(=O)c4cnn(C)c4)cc3n2CC(C)C)n1] | N-(2-(2-ethoxypyrimidin-4-yl)-1- isobutyl-1H-pyrrolo[3,2-c]pyridin- 6-yl)-1-methyl-1H-pyrazole-4- carboxamide | 420.1 | | D | D |
| 317 | 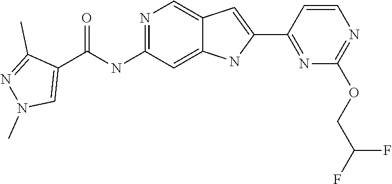 [SMILES: Cc1nn(C)cc1C(=O)Nc1cc2[nH]c(-c3ccnc(OCC(F)F)n3)cc2cn1] | N-(2-(2-(2,2- difluoroethoxy)pyrimidin-4-yl)-1H- pyrrolo[3,2-c]pyridin-6-yl)-1,3- dimethyl-1H-pyrazole-4- carboxamide | 414.2 | | D | D |
| 318 | 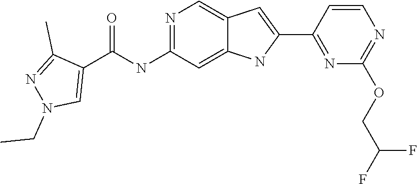 [SMILES: CCn1cc(C(=O)Nc2cc3[nH]c(-c4ccnc(OCC(F)F)n4)cc3cn2)c(C)n1] | N-(2-(2-(2,2- difluoroethoxy)pyrimidin-4-yl)-1H- pyrrolo[3,2-c]pyridin-6-yl)-1-methyl- 3-methyl-1H-pyrazole-4- carboxamide | 428.1 | | D | D |
| 319 | 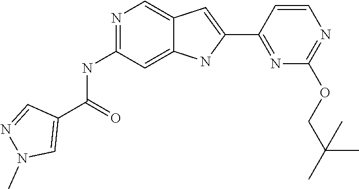 [SMILES: Cn1cc(C(=O)Nc2cc3[nH]c(-c4ccnc(OCC(C)(C)C)n4)cc3cn2)cn1] | 1-methyl-N-(2-(2- (neopentyloxy)pyrimidin-4-yl)-1H- pyrrolo[3,2-c]pyridin-6-yl)-1H- pyrazole-4-carboxamide | 406.2 | | D | D |
| 320 | 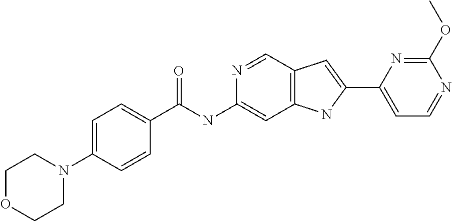 [SMILES: COc1nccc(-c2cc3cnc(NC(=O)c4ccc(N5CCOCC5)cc4)cc3[nH]2)n1] | N-(2-(2-methoxypyrimidin-4-yl)- 1H-pyrrolo[3,2-c]pyridin-6-yl)-4- morpholinobenzamide | 431.2 | | D | D |
| 321 | 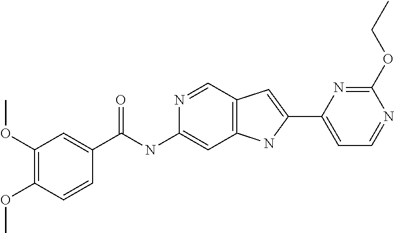 [SMILES: CCOc1nccc(-c2cc3cnc(NC(=O)c4ccc(OC)c(OC)c4)cc3[nH]2)n1] | N-(2-(2-ethoxypyrimidin-4-yl)-1H- pyrrolo[3,2-c]pyridin-6-yl)-3,4- dimethoxybenzamide | 420.2 | | D | D |
| 322 | 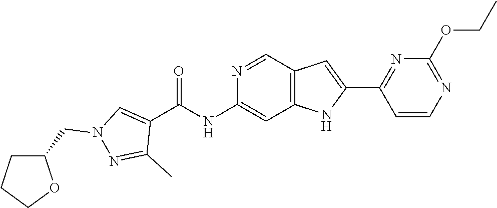 [SMILES: CCOc1nccc(-c2cc3cnc(NC(=O)c4cn(C[C@H]5CCCO5)nc4C)cc3[nH]2)n1] | (S)-N-(2-(2-ethoxypyrimidin-4-yl)- 1H-pyrrolo[3,2-c]pyridin-6-yl)-3- methyl-1-((tetrahydrofuran-2- yl)methyl)-1H-pyrazole-4- carboxamide | 448.0 | | D | D |
| 323 | 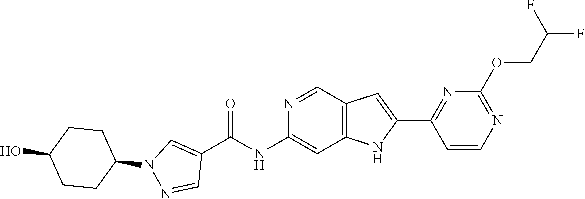 [SMILES: O=C(Nc1cc2[nH]c(-c3ccnc(OCC(F)F)n3)cc2cn1)c1cnn([C@H]2CC[C@@H](O)CC2)c1] | N-(2-(2-(2,2- difluoroethoxy)pyrimidin-4-yl)-1H- pyrrolo[3,2-c]pyridin-6-yl)-1- ((1s,4s)-4-hydroxycyclohexyl)- 1H-pyrazole-4-carboxamide | 484.1 | | D | D |
| 324 | 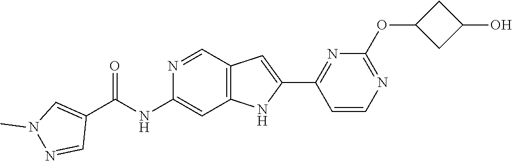 [SMILES: Cn1cc(C(=O)Nc2cc3[nH]c(-c4ccnc(OC5CC(O)C5)n4)cc3cn2)cn1] | N-(2-(2-(3- hydroxycyclobutoxy)pyrimidin-4- yl)-1H-pyrrolo[3,2-c]pyridin-6-yl)- 1-methyl-1H-pyrazole-4- carboxamide | 406.0 | | D | D |
| 325 | 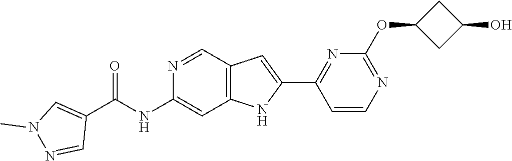 [SMILES: Cn1cc(C(=O)Nc2cc3[nH]c(-c4ccnc(O[C@H]5C[C@@H](O)C5)n4)cc3cn2)cn1] | N-(2-(2-((1s,4s)-4- hydroxycyclohexyloxy)pyrimidin- 4-yl)-1H-pyrrolo[3,2-c]pyridin-6- yl)-1-methyl-1H-pyrazole-4- carboxamide | 434.0 | | D | D |
| 326 | 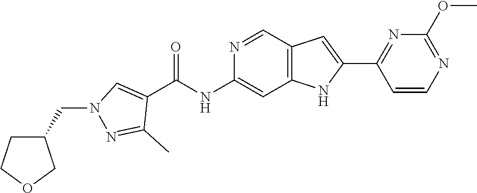 [SMILES: COc1nccc(-c2cc3cnc(NC(=O)c4cn(C[C@H]5CCOC5)nc4C)cc3[nH]2)n1] | (S)-N-(2-(2-methoxypyrimidin-4- yl)-1H-pyrrolo[3,2-c]pyridin-6-yl)- 3-methyl-1-((tetrahydrofuran-3- yl)methyl)-1H-pyrazole-4- carboxamide | 434.0 | | D | D |
| 327 | 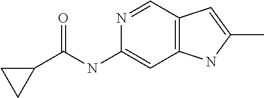 [SMILES: Cc1cc2cnc(NC(=O)C3CC3)cc2[nH]1] | N-(2-methyl-1H-pyrrolo[3,2- c]pyridin-6- yl)cyclopropanecarboxamide | 216.3 | | D | D |
| 328 | 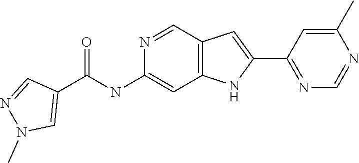 [SMILES: Cc1cc(-c2cc3cnc(NC(=O)c4cnn(C)c4)cc3[nH]2)ncn1] | 1-methyl-N-(2-(6-methylpyrimidin- 4-yl)-1H-pyrrolo[3,2-c]pyridin-6- yl)-1H-pyrazole-4-carboxamide | 334.0 | | D | D |
| 329 | 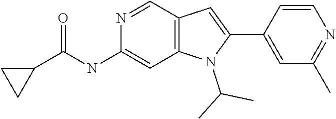 [SMILES: Cc1cc(-c2cc3cnc(NC(=O)C4CC4)cc3n2C(C)C)ccn1] | N-(1-isopropyl-2-(2-methylpyridin- 4-yl)-1H-pyrrolo[3,2-c]pyridin-6- yl)cyclopropanecarboxamide | 335.3 | | D | D |
| 330 | 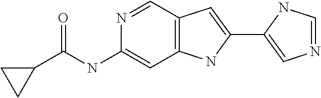 [SMILES: O=C(Nc1cc2[nH]c(-c3cnc[nH]3)cc2cn1)C1CC1] | N-(2-(1H-imidazol-5-yl)-1H- pyrrolo[3,2-c]pyridin-6- yl)cyclopropanecarboxamide | 268.1 | | D | D |
| 331 | 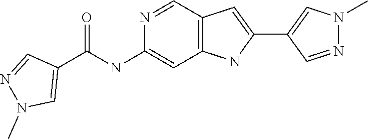 [SMILES: Cn1cc(C(=O)Nc2cc3[nH]c(-c4cnn(C)c4)cc3cn2)cn1] | 1-methyl-N-(2-(1-methyl-1H- pyrazol-4-yl)-1H-pyrrolo[3,2- c]pyridin-6-yl)-1H-pyrazole-4- carboxamide | 322.2 | | D | D |
| 332 | 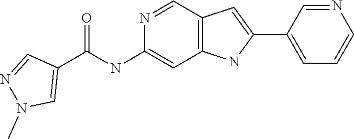 [SMILES: Cn1cc(C(=O)Nc2cc3[nH]c(-c4cccnc4)cc3cn2)cn1] | 1-methyl-N-(2-(pyridin-3-yl)-1H- pyrrolo[3,2-c]pyridin-6-yl)-1H- pyrazole-4-carboxamide | 319.2 | | D | D |
| 333 | 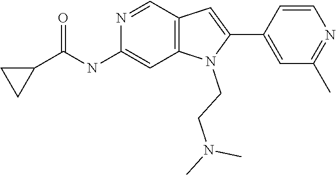 [SMILES: Cc1cc(-c2cc3cnc(NC(=O)C4CC4)cc3n2CCN(C)C)ccn1] | N-(1-(2-(dimethylamino)ethyl)-2- (2-methylpyridin-4-yl)-1H- pyrrolo[3,2-c]pyridin-6- yl)cyclopropanecarboxamide | 364.2 | | D | D |
| 334 | 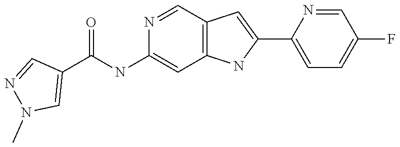 [SMILES: Cn1cc(C(=O)Nc2cc3[nH]c(-c4ccc(F)cn4)cc3cn2)cn1] | N-(2-(5-fluoropyridin-2-yl)-1H- pyrrolo[3,2-c]pyridin-6-yl)-1- methyl-1H-pyrazole-4- carboxamide | 337.2 | | D | D |
| 335 | 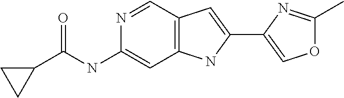 [SMILES: Cc1nc(-c2cc3cnc(NC(=O)C4CC4)cc3[nH]2)co1] | N-(2-(2-methyloxazol-4-yl)-1H- pyrrolo[3,2-c]pyridin-6- yl)cyclopropanecarboxamide | 283.2 | | D | D |
| 336 | 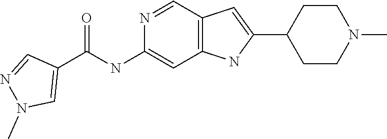 [SMILES: CN1CCC(c2cc3cnc(NC(=O)c4cnn(C)c4)cc3[nH]2)CC1] | 1-methyl-N-(2-(1-methylpiperidin- 4-yl)-1H-pyrrolo[3,2-c]pyridin-6- yl)-1H-pyrazole-4-carboxamide | 339.2 | | D | D |
| 337 | 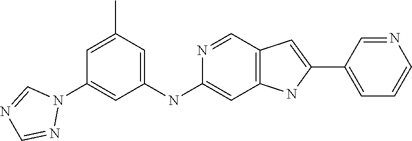 [SMILES: Cc1cc(Nc2cc3[nH]c(-c4cccnc4)cc3cn2)cc(-n2cncn2)c1] | N-(3-methyl-5-(1H-1,2,4-triazol-1- yl)phenyl)-2-(pyridin-3-yl)-1H- pyrrolo[3,2-c]pyridin-6-amine | 368.3 | | D | D |
| 338 | 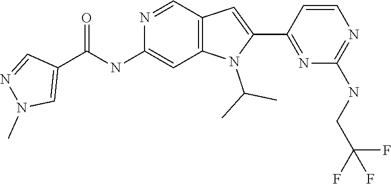 [SMILES: CC(C)n1c(-c2ccnc(NCC(F)(F)F)n2)cc2cnc(NC(=O)c3cnn(C)c3)cc21] | N-(1-isopropyl-2-(2-(2,2,2- trifluoroethylamino)pyrimidin-4-yl)- 1H-pyrrolo[3,2-c]pyridin-6-yl)-1- methyl-1H-pyrazole-4- carboxamide | 459.2 | | D | D |
| 339 | 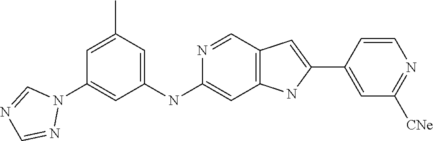 | 4-(6-(3-methyl-5-(1H-1,2,4-triazol- 1-yl)phenylamino)-1H-pyrrolo[3,2- c]pyridin-2-yl)picolinonitrile | 393.3 | | D | D |
| 340 | 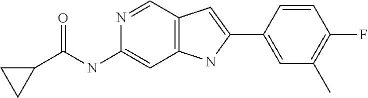 [SMILES: Cc1cc(-c2cc3cnc(NC(=O)C4CC4)cc3[nH]2)ccc1F] | N-(2-(4-fluoro-3-methylphenyl)- 1H-pyrrolo[3,2-c]pyridin-6- yl)cyclopropanecarboxamide | 310.2 | | D | D |
| 341 | 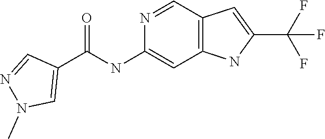 [SMILES: Cn1cc(C(=O)Nc2cc3[nH]c(C(F)(F)F)cc3cn2)cn1] | 1-methyl-N-(2-(tirfluoromethyl)-1H- pyrrolo[3,2-c]pyridin-6-yl)-1H- pyrazole-4-carboxamide | 310.2 | | D | D |
| 342 | 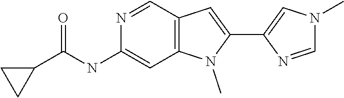 [SMILES: Cn1cnc(-c2cc3cnc(NC(=O)C4CC4)cc3n2C)c1] | N-(1-methyl-2-(1-methyl-1H- imidazol-4-yl)-1H-pyrrolo[3,2- c]pyridin-6- yl)cyclopropanecarboxamide | 296.1 | A | | |
| 343 | 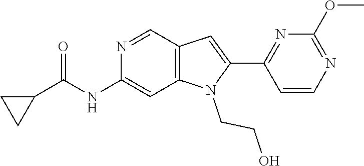 [SMILES: COc1nccc(-c2cc3cnc(NC(=O)C4CC4)cc3n2CCO)n1] | N-(1-(2-hydroxyethyl)-2-(2- methoxypyrimidin-4-yl)-1H- pyrrolo[3,2-c]pyridin-6- yl)cyclopropanecarboxamide | 354.3 | A | | |
| 344 | 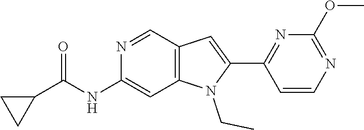 [SMILES: CCn1c(-c2ccnc(OC)n2)cc2cnc(NC(=O)C3CC3)cc21] | N-(1-ethyl-2-(2-methoxypyrimidin- 4-yl)-1H-pyrrolo[3,2-c]pyridin-6- yl)cyclopropanecarboxamide | 338.3 | B | | |
| 345 | 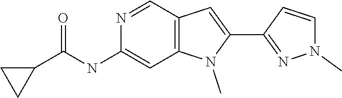 [SMILES: Cn1ccc(-c2cc3cnc(NC(=O)C4CC4)cc3n2C)n1] | N-(1-methyl-2-(1-methyl-1H- pyrazol-3-yl)-1H-pyrrolo[3,2- c]pyridin-6- yl)cycloproapnecarboxamide | 296.3 | A | | |
| 346 | 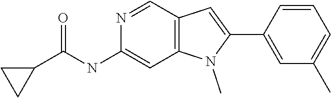 [SMILES: Cc1cccc(-c2cc3cnc(NC(=O)C4CC4)cc3n2C)c1] | N-(1-methyl-2-m-tolyl-1H- pyrrolo[3,2-c]pyridin-6- yl)cyclopropanecarboxamide | 306.3 | A | | |
| 347 | 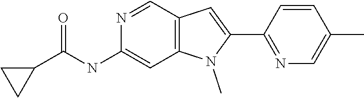 [SMILES: Cc1ccc(-c2cc3cnc(NC(=O)C4CC4)cc3n2C)nc1] | N-(1-methyl-2-(5-methylpyridin-2- yl)-1H-pyrrolo[3,2-c]pyridin-6- yl)cyclopropanecarboxamide | 307.2 | A | | |
| 348 | 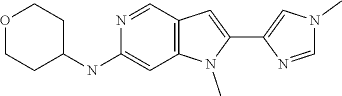 [SMILES: Cn1cnc(-c2cc3cnc(NC4CCOCC4)cc3n2C)c1] | 1-methyl-2-(1-methyl-1H-imidazol- 4-yl)-N-(tetrahydro-2H-pyran-4- yl)-1H-pyrrolo[3,2-c]pyridin-6- amine | 312.2 | A | | |
| 349 | 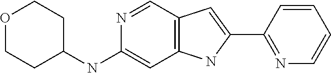 [SMILES: c1ccc(-c2cc3cnc(NC4CCOCC4)cc3[nH]2)nc1] | 2-(pyridin-2-yl)-N-(tetrahydro-2H- pyran-4-yl)-1H-pyrrolo[3,2- c]pyridin-6-amine | 295.2 | A | | |
| 350 | 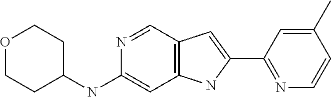 [SMILES: Cc1ccnc(-c2cc3cnc(NC4CCOCC4)cc3[nH]2)c1] | 2-(4-methylpyridin-2-yl)-N- (tetrahydro-2H-pyran-4-yl)-1H- pyrrolo[3,2-c]pyridin-6-amine | 309.3 | A | | |
| 351 | 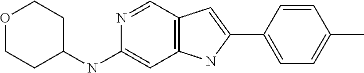 [SMILES: Cc1ccc(-c2cc3cnc(NC4CCOCC4)cc3[nH]2)cc1] | N-(tetrahydro-2H-pyran-4-yl)-2-p- tolyl-1H-pyrrolo[3,2-c]pyridin-6- amine | 308.3 | A | | |
| 352 | 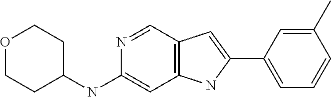 [SMILES: Cc1cccc(-c2cc3cnc(NC4CCOCC4)cc3[nH]2)c1] | N-(tetrahydro-2H-pyran-4-yl)-2-m- tolyl-1H-pyrrolo[3,2-c]pyridin-6- amine | 308.3 | A | | |
| 353 | 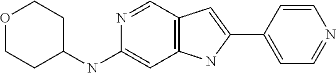 [SMILES: c1cc(-c2cc3cnc(NC4CCOCC4)cc3[nH]2)ccn1] | 2-(pyridin-4-yl)-N-(tetrahydro-2H- pyran-4-yl)-1H-pyrrolo[3,2- c]pyridin-6-amine | 295.1 | C | | |
| 354 | 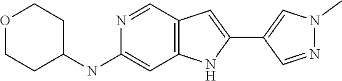 [SMILES: Cn1cc(-c2cc3cnc(NC4CCOCC4)cc3[nH]2)cn1] | 2-(1-methyl-1H-pyrazol-4-yl)-N- (tetrahydro-2H-pyran-4-yl)-1H- pyrrolo[3,2-c]pyridin-6-amine | 298.2 | A | | |
| 355 | 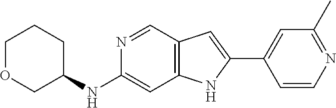 [SMILES: Cc1cc(-c2cc3cnc(N[C@@H]4CCCOC4)cc3[nH]2)ccn1] | (S)-2-(2-methylpyridin-4-yl)-N- (tetrahydro-2H-pyran-3-yl)-1H- pyrrolo[3,2-c]pyridin-6-amine | 309.1 | A | | |
| 356 | 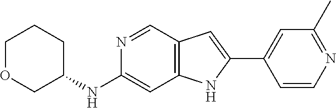 [SMILES: Cc1cc(-c2cc3cnc(N[C@H]4CCCOC4)cc3[nH]2)ccn1] | (R)-2-(2-methylpyridin-4-yl)-N- (tetrahydro-2H-pyran-3-yl)-1H- pyrrolo[3,2-c]pyridin-6-amine | 309.1 | C | | |
| 357 | 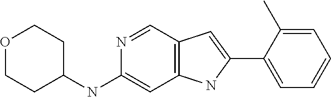 [SMILES: Cc1ccccc1-c1cc2cnc(NC3CCOCC3)cc2[nH]1] | N-(tetrahydro-2H-pyran-4-yl)-2-o- tolyl-1H-pyrrolo[3,2-c]pyridin-6- amine | 308.3 | A | | |
| 358 | 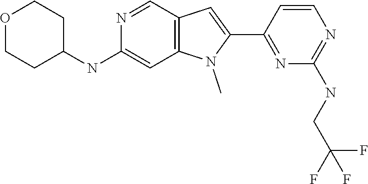 [SMILES: Cn1c(-c2ccnc(NCC(F)(F)F)n2)cc2cnc(NC3CCOCC3)cc21] | 1-methyl-N-(tetrahydro-2H-pyran- 4-yl)-2-(2-(2,2,2- trifluoroethylamino)pyrimidin-4-yl)- 1H-pyrrolo[3,2-c]pyridin-6-amine | 407.1 | C | | |
| 359 | 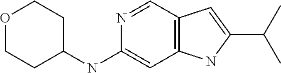 [SMILES: CC(C)c1cc2cnc(NC3CCOCC3)cc2[nH]1] | 2-isopropyl-N-(tetrahydro-2H- pyran-4-yl)-1H-pyrrolo[3,2- c]pyridin-6-amine | 260.3 | A | | |
| 360 | 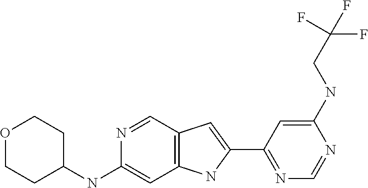 [SMILES: FC(F)(F)CNc1cc(-c2cc3cnc(NC4CCOCC4)cc3[nH]2)ncn1] | N -(tetrahydro-2H-pyran-4-yl)-2- (6-(2,2,2- trifluoroethylamino)pyrimidin-4-yl)- 1H-pyrrolo[3,2-c]pyridin-6-amine | 393.3 | C | | |
| 361 | 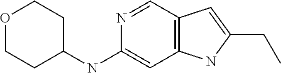 [SMILES: CCc1cc2cnc(NC3CCOCC3)cc2[nH]1] | 2-ethyl-N-(tetrahydro-2H-pyran-4- yl)-1H-pyrrolo[3,2-c]pyridin-6- amine | 246.3 | A | | |
| 362 | 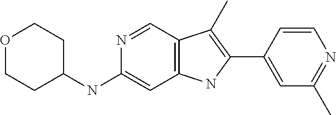 [SMILES: Cc1cc(-c2[nH]c3cc(NC4CCOCC4)ncc3c2C)ccn1] | 3-methyl-2-(2-methylpyridin-4-yl)- N-(tetrahydro-2H-pyran-4-yl)-1H- pyrrolo[3,2-c]pyridin-6-amine | 323.2 | A | | |
| 363 | 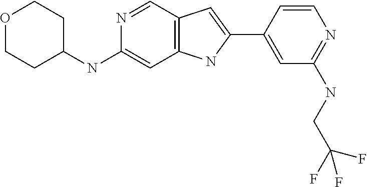 [SMILES: FC(F)(F)CNc1cc(-c2cc3cnc(NC4CCOCC4)cc3[nH]2)ccn1] | N-(tetrahydro-2H-pyran-4-yl)-2- (2-(2,2,2- trifluoroethylamino)pyridin-4-yl)- 1H-pyrrolo[3,2-c]pyridin-6-amine | 392.3 | C | | |
| 364 | 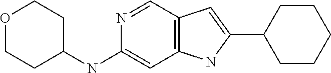 [SMILES: c1nc(NC2CCOCC2)cc2[nH]c(C3CCCCC3)cc12] | 2-cyclohexyl-N-(tetrahydro-2H- pyran-4-yl)-1H-pyrrolo[3,2- c]pyridin-6-amine | 300.3 | | | |
| 365 | 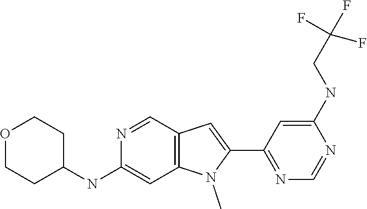 [SMILES: Cn1c(-c2cc(NCC(F)(F)F)ncn2)cc2cnc(NC3CCOCC3)cc21] | 1-methyl-N-(tetrahydro-2H-pyran- 4-yl)-2-(6-(2,2,2- trifluoroethyl)amino)pyrimidin-4-yl)- 1H-pyrrolo[3,2-c]pyridin-6-amine | 407.1 | A | | |
| 366 | 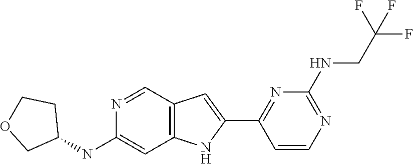 [SMILES: FC(F)(F)CNc1nccc(-c2cc3cnc(N[C@H]4CCOC4)cc3[nH]2)n1] | (R)-N-(tetrahydrofuran-3-yl)-2-(2- (2,2,2- trifluoroethylamino)pyrimidin-4-yl)- 1H-pyrrolo[3,2-c]pyridin-6-amine | 379.2 | C | | |
| 367 | 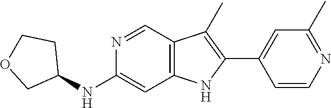 [SMILES: Cc1cc(-c2[nH]c3cc(N[C@@H]4CCOC4)ncc3c2C)ccn1] | (S)-3-methyl-2-(2-methylpyridin-4- yl)-N-(tetrahydrofuran-3-yl)-1H- pyrrolo[3,2-c]pyridin-6-amine | 309.1 | A | | |
| 368 | 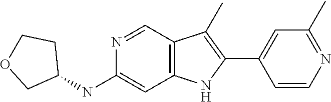 [SMILES: Cc1cc(-c2[nH]c3cc(N[C@H]4CCOC4)ncc3c2C)ccn1] | (R)-3-methyl-2-(2-methylpyridin-4- yl)-N-(tetrahydrofuran-3-yl)-1H- pyrrolo[3,2-c]pyridin-6-amine | 309.1 | A | | |
| 369 | 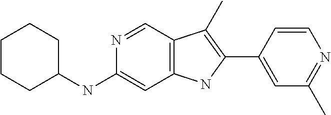 [SMILES: Cc1cc(-c2[nH]c3cc(NC4CCCCC4)ncc3c2C)ccn1] | N-cyclohexyl-3-methyl-2-(2- methylpyridin-4-yl)-1H-pyrrolo[3,2- c]pyridin-6-amine | 321.3 | A | | |
| 370 | 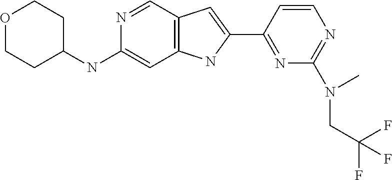 [SMILES: CN(CC(F)(F)F)c1nccc(-c2cc3cnc(NC4CCOCC4)cc3[nH]2)n1] | 2-(2-(methyl(2,2,2- trifluoroethyl)amino)pyrimidin-4- yl)-N-(tetrahydro-2H-pyran-4-yl)- 1H-pyrrolo[3,2-c]pyridin-6-amine | 407.2 | B | | |
| 371 | 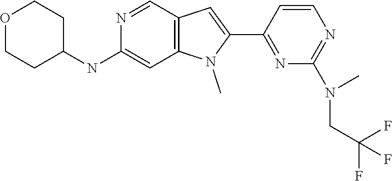 [SMILES: CN(CC(F)(F)F)c1nccc(-c2cc3cnc(NC4CCOCC4)cc3n2C)n1] | 1-methyl-2-(2-(methyl(2,2,2- trifluoroethyl)amino)pyrimidin-4- yl)-N-(tetrahydro-2H-pyran-4-yl)- 1H-pyrrolo[3,2-c]pyridin-6-amine | 421.2 | A | | |
| 372 | 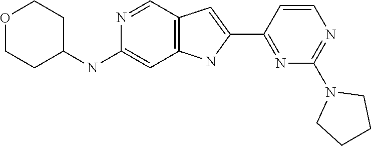 [SMILES: c1cc(-c2cc3cnc(NC4CCOCC4)cc3[nH]2)nc(N2CCCC2)n1] | 2-(2-(pyrrolidin-1-yl)pyrimidin-4- yl)-N-(tetrahydro-2H-pyran-4-yl)- 1H-pyrrolo[3,2-c]pyridin-6-amine | 365.3 | C | | |
| 373 | 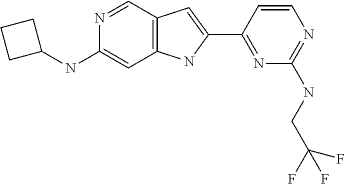 [SMILES: FC(F)(F)CNc1nccc(-c2cc3cnc(NC4CCC4)cc3[nH]2)n1] | N-cyclobutyl-2-(2-(2,2,2- trifluoroethylamino)pyrimidin-4-yl)- 1H-pyrrolo[3,2-c]pyridin-6-amine | 363.3 | B | | |
| 374 | 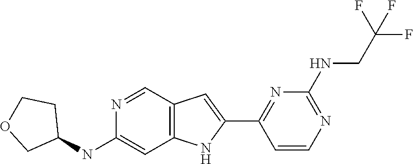 [SMILES: FC(F)(F)CNc1nccc(-c2cc3cnc(N[C@@H]4CCOC4)cc3[nH]2)n1] | (S)-N-(tetrahydrofuran-3-yl)-2-(2- (2,2,2- trifluoroethylamino)pyrimidin-4-yl)- 1H-pyrrolo[3,2-c]pyridin-6-amine | 379.2 | B | | |
| 375 | 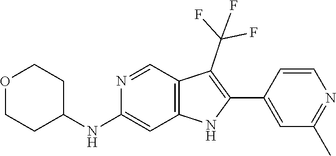 [SMILES: Cc1cc(-c2[nH]c3cc(NC4CCOCC4)ncc3c2C(F)(F)F)ccn1] | 2-(2-methylpyridin-4-yl)-N- (tetrahydro-2H-pyran-4-yl)-3- (trifluoromethyl)-1H-pyrrolo[3,2- c]pyridin-6-amine | 377.3 | A | | |
| 376 | 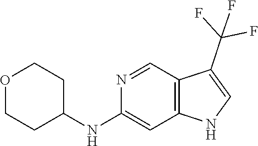 [SMILES: FC(F)(F)c1c[nH]c2cc(NC3CCOCC3)ncc12] | N-(tetrahydro-2H-pyran-4-yl)-3- (trifluoromethyl)-1H-pyrrolo[3,2- c]pyridin-6-amine | 286.2 | A | | |
| 377 | 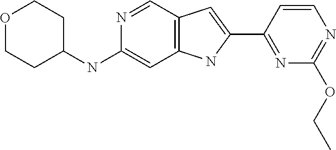 [SMILES: CCOc1nccc(-c2cc3cnc(NC4CCOCC4)cc3[nH]2)n1] | 2-(2-ethoxypyrimidin-4-yl)-N- (tetrahydro-2H-pyran-4-yl)-1H- pyrrolo[3,2-c]pyridin-6-amine | 340.2 | A | | |
| 378 | 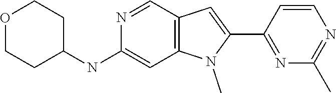 [SMILES: Cc1nccc(-c2cc3cnc(NC4CCOCC4)cc3n2C)n1] | 1-methyl-2-(2-methylpyrimidin-4- yl)-N-(tetrahydro-2H-pyran-4-yl)- 1H-pyrrolo[3,2-c]pyridin-6-amine | 324.2 | A | | |
| 379 | 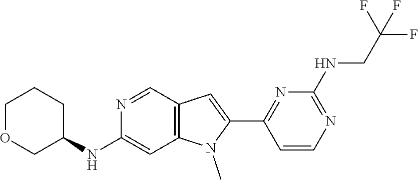 [SMILES: Cn1c(-c2ccnc(NCC(F)(F)F)n2)cc2cnc(N[C@@H]3CCCOC3)cc21] | (S)-1-methyl-N-(tetrahydro-2H- pyran-3-yl)-2-(2-(2,2,2- trifluoroethylamino)pyrimidin-4-yl)- 1H-pyrrolo[3,2-c]pyridin-6-amine | 407.1 | A | | |
| 380 | 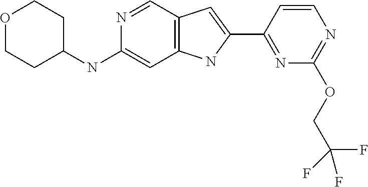 [SMILES: FC(F)(F)COc1nccc(-c2cc3cnc(NC4CCOCC4)cc3[nH]2)n1] | N-(tetrahydro-2H-pyran-4-yl)-2- (2-(2,2,2-trifluoroethoxy)pyrimidin- 4-yl)-1H-pyrrolo[3,2-c]pyridin-6- amine | 394.2 | A | | |
| 381 | 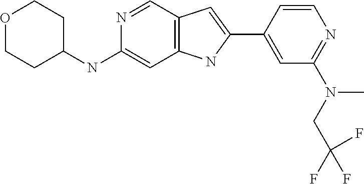 [SMILES: CN(CC(F)(F)F)c1cc(-c2cc3cnc(NC4CCOCC4)cc3[nH]2)ccn1] | 2-(2-(methyl(2,2,2- trifluoroethyl)amino)pyridin-4-yl)- N-(tetrahydro-2H-pyran-4-yl)-1H- pyrrolo[3,2-c]pyridin-6-amine | 406.2 | A | | |
| 382 | 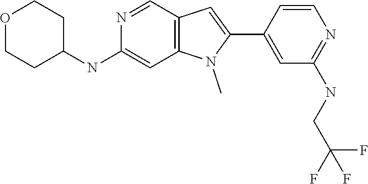 [SMILES: Cn1c(-c2ccnc(NCC(F)(F)F)c2)cc2cnc(NC3CCOCC3)cc21] | 1-methyl-N-(tetrahydro-2H-pyran- 4-yl)-2-(2-(2,2,2- trifluoroethylamino)pyridin-4-yl)- 1H-pyrrolo[3,2-c]pyridin-6-amine | 406.2 | A | | |
| 383 | 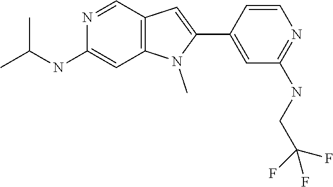 [SMILES: CC(C)Nc1cc2c(cn1)cc(-c1ccnc(NCC(F)(F)F)c1)n2C] | N-isopropyl-1-methyl-2-(2-(2,2,2- trifluoroethylamino)pyrimidin-4-yl)- 1H-pyrrolo[3,2-c]pyridin-6-amine | 365.2 | A | | |
| 384 | 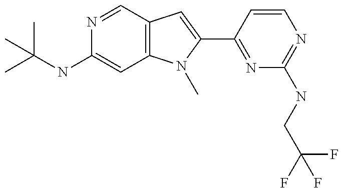 [SMILES: Cn1c(-c2ccnc(NCC(F)(F)F)n2)cc2cnc(NC(C)(C)C)cc21] | N-tert-butyl-1-methyl-2-(2-(2,2,2- trifluoroethylamino)pyrimidin-4-yl)- 1H-pyrrolo[3,2-c]pyridin-6-amine | 379.2 | A | | |
| 385 | 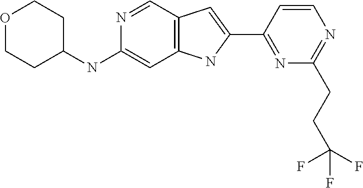 [SMILES: FC(F)(F)CCc1nccc(-c2cc3cnc(NC4CCOCC4)cc3[nH]2)n1] | N-(tetrahydro-2H-pyran-4-yl)-2- (2-(3,3,3-trifluoropropyl)pyrimidin- 4-yl)-1H-pyrrolo[3,2-c]pyridin-6- amine | 392.1 | A | | |
| 386 | 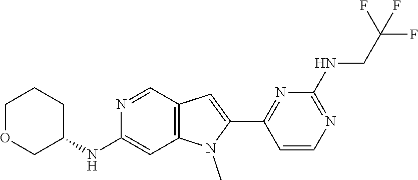 [SMILES: Cn1c(-c2ccnc(NCC(F)(F)F)n2)cc2cnc(N[C@H]3CCCOC3)cc21] | (R)-1-methyl-N-(tetrahydro-2H- pyran-3-yl)-2-(2-(2,2,2- trifluoroethyl)amino)pyrimidin-4-yl)- 1H-pyrrolo[3,2-c]pyridin-6-amine | 407.1 | A | | |
| 387 | 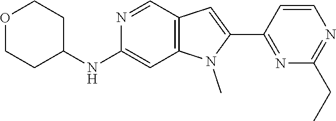 [SMILES: CCc1nccc(-c2cc3cnc(NC4CCOCC4)cc3n2C)n1] | 2-(2-ethylpyrimidin-4-yl)-1-methyl- N-(tetrahydro-2H-pyran-4-yl)-1H- pyrrolo[3,2-c]pyridin-6-amine | 338.3 | A | | |
| 388 | 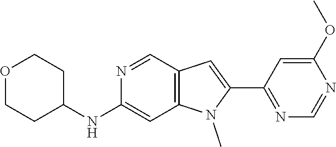 [SMILES: COc1cc(-c2cc3cnc(NC4CCOCC4)cc3n2C)ncn1] | 2-(6-methoxypyrimidin-4-yl)-1- methyl-N-(tetrahydro-2H-pyran-4- yl)-1H-pyrrolo[3,2-c]pyridin-6- amine | 340.2 | A | | |
| 389 | 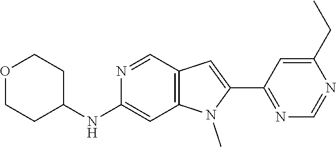 [SMILES: CCc1cc(-c2cc3cnc(NC4CCOCC4)cc3n2C)ncn1] | 2-(6-ethylpyrimidin-4-yl)-1-methyl- N-(tetrahydro-2H-pyran-4-yl)-1H- pyrrolo[3,2-c]pyridin-6-amine | 338.3 | A | | |
| 390 | 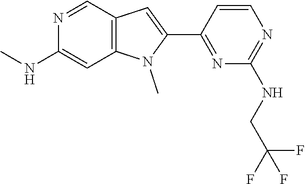 [SMILES: CNc1cc2c(cn1)cc(-c1ccnc(NCC(F)(F)F)n1)n2C] | N,1-dimethyl-2-(2-(2,2,2- trifluoroethylamino)pyrimidin-4-yl)- 1H-pyrrolo[3,2-c]pyridin-6-amine | 337.2 | A | | |
| 391 | 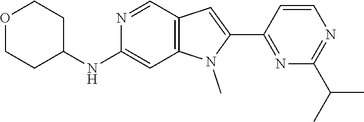 [SMILES: CC(C)c1nccc(-c2cc3cnc(NC4CCOCC4)cc3n2C)n1] | 2-(2-isopropylpyrimidin-4-yl)-1- methyl-N-(tetrahydro-2H-pyran-4- yl)-1H-pyrrolo[3,2-c]pyridin-6- amine | 352.2 | A | | |
| 392 | 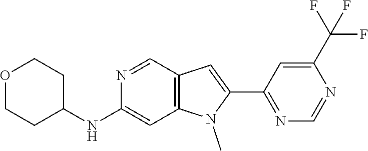 [SMILES: Cn1c(-c2cc(C(F)(F)F)ncn2)cc2cnc(NC3CCOCC3)cc21] | 1-methyl-N-(tetrahydro-2H-pyran- 4-yl)-2-(6- (trifluoromethyl)pyrimidin-4-yl)-1H- pyrrolo[3,2-c]pyridin-6-amine | 378.1 | A | | |
| 393 | 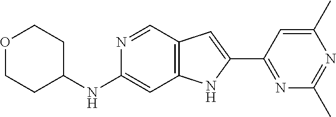 [SMILES: Cc1cc(-c2cc3cnc(NC4CCOCC4)cc3[nH]2)nc(C)n1] | 2-(2,6-dimethylpyrimidin-4-yl)-N- (tetrahydro-2H-pyran-4-yl)-1H- pyrrolo[3,2-c]pyridin-6-amine | 324.1 | A | | |
| 394 | 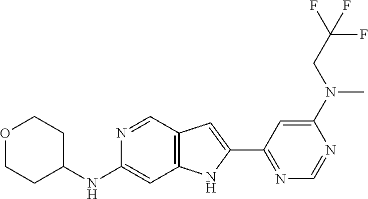 [SMILES: CN(CC(F)(F)F)c1cc(-c2cc3cnc(NC4CCOCC4)cc3[nH]2)ncn1] | 2-(6-(methyl(2,2,2- trifluoroethyl)amino)pyrimidin-4- yl)-N-(tetrahydro-2H-pyran-4-yl)- 1H-pyrrolo[3,2-c]pyridin-6-amine | 407.2 | A | | |
| 395 | 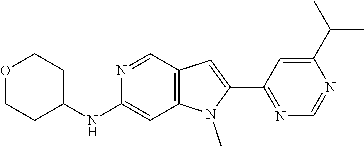 [SMILES: CC(C)c1cc(-c2cc3cnc(NC4CCOCC4)cc3n2C)ncn1] | 2-(6-isopropylpyrimidin-4-yl)-1- methyl-N-(tetrahydro-2H-pyran-4- yl)-1H-pyrrolo[3,2-c]pyridin-6- amine | 352.2 | A | | |
| 396 | 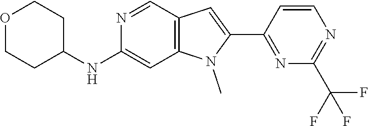 [SMILES: Cn1c(-c2ccnc(C(F)(F)F)n2)cc2cnc(NC3CCOCC3)cc21] | 1-methyl-N-(tetrahydro-2H-pyran- 4-yl)-2-(2- (trifluoromethyl)pyrimidin-4-yl)-1H- pyrrolo[3,2-c]pyridin-6-amine | 378.1 | A | | |
| 397 | 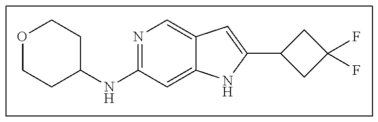 [SMILES: FC1(F)CC(c2cc3cnc(NC4CCOCC4)cc3[nH]2)C1] | 2-(3,3-difluorocyclobutyl)-N- (tetrahydro-2H-pyran-4-yl)-1H- pyrrolo[3,2-c]pyridin-6-amine | | | E | E |

A number of references have been cited, the disclosures of each of which are incorporated herein by reference in their entirety.

What is claimed is:

1. A compound according to Formula (I) 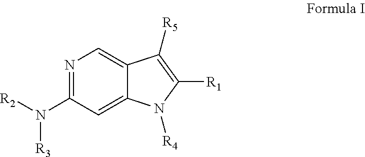
and pharmaceutically acceptable salts, tautomers, isotopologues, or stereoisomers thereof, wherein
R₁ is chosen from H, substituted or unsubstituted C₁-C₄ alkyl, substituted or unsubstituted 3-6 membered cycloalkyl, substituted or unsubstituted 6-membered heterocyclyl, substituted or unsubstituted 6-membered aryl, substituted or unsubstituted 5-6 membered heteroaryl, and —CN;
R₂ is chosen from H and —CH₃;
R₃ is chosen from H, substituted or unsubstituted C₁-C₄ alkyl, substituted or unsubstituted 4-6 membered cycloalkyl, substituted or unsubstituted 4-9 membered heterocyclyl, substituted or unsubstituted 6-membered aryl, substituted or unsubstituted 5-membered heteroaryl, —CH₂-cyclopropyl, —C(═O)—R₆, —C(═O)CH₂-R₇, —C(═O)—O—R₈, —C(═O)NR₉-R₁₀, —C(═N)R₁₁, and —S(═O)₂-R₁₂;
R₄ is chosen from H and substituted or unsubstituted C₁-C₄ alkyl;
R₅ group chosen from H and substituted or unsubstituted C₁-C₄ alkyl;
R₆ is chosen from substituted or unsubstituted C₁-C₄ alkyl, substituted or unsubstituted 3-7 membered cycloalkyl, substituted or unsubstituted 5-6 membered heterocyclyl, substituted or unsubstituted 6 membered aryl, and substituted or unsubstituted 5-9 membered heteroaryl;
R₇ is chosen from substituted or unsubstituted 5 membered cycloalkyl, substituted or unsubstituted 4-6 membered heterocyclyl, and —N(CH₃)₂;
R₈ is chosen from substituted or unsubstituted C₁ alkyl, substituted or unsubstituted 5-6 membered cycloalkyl, and substituted or unsubstituted 5 membered heterocyclyl;
R₉ and R₁₀ are independently chosen from H, substituted or unsubstituted C₁ alkyl, substituted or unsubstituted 5-6 membered cycloalkyl, or R₉ and R₁₀ are taken together with the N to which they are attached and form a substituted or unsubstituted 5 membered heterocyclyl;
R₁₁ is a substituted or unsubstituted 3 membered cycloalkyl;
R₁₂ is a substituted or unsubstituted 3 membered cycloalkyl; and
wherein when R₁ is not a substituted or unsubstituted 5-6 membered heteroaryl, then R₃ is chosen from substituted or unsubstituted 4-9 membered heterocyclyl, substituted or unsubstituted 6-membered aryl, substituted or unsubstituted 5-membered heteroaryl, —C(═O)—R₆, —C(═O)—O—R₈, —C(═O)NR₉-R₁₀,—S(═O)₂-R₁₂.

2. The compound according to claim 1, wherein R₁ is chosen from substituted or unsubstituted C₁-C₄ cycloalkyl, substituted or unsubstituted 6 membered aryl, substituted or unsubstituted 5-6 membered heteroaryl, —CF₃, and —CN.

3. The compound according to any of claims 1-2, wherein R₁ is chosen from a substituted or unsubstituted 4-6 membered cycloalkyl, substituted or unsubstituted 6 membered aryl, and substituted or unsubstituted 5 membered heteroaryl.

4. The compound according to any of claims 1-3, wherein R₃ is chosen from substituted or unsubstituted C₁-C₄ alkyl, substituted or unsubstituted 4-6 membered cycloalkyl, substituted or unsubstituted 4-9 membered heterocyclyl, substituted or unsubstituted 6 membered aryl, substituted or unsubstituted 5 membered heteroaryl, —CH₂-cyclopropyl, and —C(═O)—R₆; and wherein R₆ is chosen from substituted or unsubstituted 3-7 membered cycloalkyl, and substituted or unsubstituted 5-9 membered heteroaryl.

5. The compound according to any of claims 1-4, wherein R₃ is chosen from substituted or unsubstituted C₁-C₄ alkyl, substituted or unsubstituted 4 membered cycloalkyl, substituted or unsubstituted 5-6 membered heterocyclyl, substituted or unsubstituted 6 membered aryl, substituted or unsubstituted 5 membered heteroaryl, and —CH₂-cyclopropyl.

6. The compound according to any of claims 1-4, wherein R₃ is =C(═O)—R₆; and wherein R₆ is chosen from substituted or unsubstituted C₁-C₄ alkyl, substituted or unsubstituted 3-7 membered cycloalkyl, substituted or unsubstituted 5-6 membered heterocyclyl, substituted or unsubstituted 6 membered aryl, and substituted or unsubstituted 5-9 membered heteroaryl.

7. The compound according to any of claims 1-6, wherein R₂ is H.

8. A compound according to Formula (II) 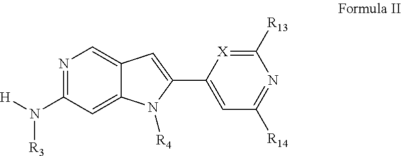
and pharmaceutically acceptable salts, tautomers, isotopologues, or stereoisomers thereof, wherein
X is chosen from N or CH;
R₃ is chosen from substituted or unsubstituted C₁-C₄ alkyl, substituted or unsubstituted 4 membered cycloalkyl, substituted or unsubstituted 5-6 membered heterocyclyl, substituted or unsubstituted 6 membered aryl, substituted or unsubstituted 5 membered heteroaryl, —CH₂-cyclopropyl, and —C(═O)—R₆;
R₄ is chosen from H and —CH₃;
R₆ is chosen from substituted or unsubstituted 3 membered cycloalkyl and substituted or unsubstituted 5 membered heteroaryl;
R₁₃ and R₁₄ are each independently chosen from H, substituted or unsubstituted C₁-C₄ alkyl, substituted or unsubstituted C₁-C₂ alkoxyl, substituted or unsubstituted amino, substituted or unsubstituted 5 membered heterocyclyl, and —CN.

9. The compound according to claim 8, wherein R₃ is chosen from substituted or unsubstituted 6 membered heterocyclyl and —C(═O)—R₆.

10. The compound according to claims 8-9, wherein R₁₃ and R₁₄ are independently chosen from H, substituted or unsubstituted C1 alkyl, and substituted or unsubstituted amino.

11. A compound according to Formula III 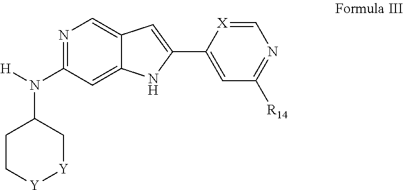
and pharmaceutically acceptable salts, tautomers, isotopologues, or stereoisomers thereof, wherein
X is chosen from N or CH;
R₁₄ is chosen from H, substituted or unsubstituted C₁-C₂ alkyl, substituted or unsubstituted C₁-C₂ alkoxyl, and substituted or unsubstituted amino; and
each Y is independently chosen from CH₂ and O.

12. The compound according to claim 11, wherein R₁₄ is chosen from substituted or unsubstituted C₁ alkyl and substituted or unsubstituted amino.

13. The compound according to claims 11-12, wherein R₁₄ is —NH(CH₂)—CF₃.

14. A compound chosen from:
N-(tetrahydro-2H-pyran-4-yl)-2-(2-(2,2,2-trifluoroethylamino)pyrimidin-4-yl)-1H-pyrrolo[3,2-c]pyridin-6-amine;
N-(3-methyl-5-(1H-1,2,4-triazol-1-yl)phenyl)-2-(2-(2,2,2-trifluoroethylamino)pyrimidin-4-yl)-1H-pyrrolo[3,2-c]pyridin-6-amine;
N-(3-methyl-2-(2-methylpyridin-4-yl)-1H-pyrrolo[3,2-c]pyridin-6-yl)cyclopropanecarboxamide;
1-methyl-N-(2-(2-(2,2,2-trifluoroethylamino)pyrimidin-4-yl)-1H-pyrrolo[3,2-c]pyridin-6-yl)-1H-pyrazole-4-carboxamide;
N-(2-(2-methylpyridin-4-yl)-1H-pyrrolo[3,2-c]pyridin-6-yl)bicyclo[3.1.0]hexane-6-carboxamide;
6-(phenylamino)-1H-pyrrolo[3,2-c]pyridine-2-carbonitrile;
1,3-dimethyl-N-(2-(2-(2,2,2-trifluoroethylamino)pyrimidin-4-yl)-1H-pyrrolo[3,2-c]pyridin-6-yl)-1H-pyrazole-4-carboxamide;
N-(2-(2-(cyclopropylamino)pyridin-4-yl)-1H-pyrrolo[3,2-c]pyridin-6-yl)cyclopropanecarboxamide;
N-(2-(2-methylpyridin-4-yl)-1H-pyrrolo[3,2-c]pyridin-6-yl)cyclopropanecarboxamide;
(1s,3s)-N-(2-(2-ethoxypyrimidin-4-yl)-1H-pyrrolo[3,2-c]pyridin-6-yl)-3-hydroxycyclobutanecarboxamide;
N-(2-(trifluoromethyl)-1H-pyrrolo[3,2-c]pyridin-6-yl)cyclopropanecarboxamide;
2-(2-methylpyridin-4-yl)-N-(tetrahydro-2H-pyran-4-yl)-1H-pyrrolo[3,2-c]pyridin-6-amine;
(R)-1-methyl-N-(2-(2-(1,1,1-trifluoropropan-2-ylamino)pyrimidin-4-yl)-1H-pyrrolo[3,2-c]pyridin-6-yl)-1H-pyrazole-4-carboxamide;
N-(2-(2-(2,2,2-trifluoroethoxy)pyrimidin-4-yl)-1H-pyrrolo[3,2-c]pyridin-6-yl)cyclopropanecarboxamide;
6-(3-morpholinophenylamino)-1H-pyrrolo[3,2-c]pyridine-2-carbonitrile;
6-(3-(tetrahydro-2H-pyran-4-yl)phenylamino)-1H-pyrrolo[3,2-c]pyridine-2-carbonitrile;
N-(2-(2-methoxypyrimidin-4-yl)-1-methyl-1H-pyrrolo[3,2-c]pyridin-6-yl)cyclopropanecarboxamide;
N-(2-(2-(2-methoxyethylamino)pyridin-4-yl)-1H-pyrrolo[3,2-c]pyridin-6-yl)cyclopropanecarboxamide;
2-methyl-N-(2-(2-methylpyridin-4-yl)-1H-pyrrolo[3,2-c]pyridin-6-yl)cyclopropanecarboxamide;
N-(3-methoxyphenyl)-2-(1H-pyrazol-4-yl)-1H-pyrrolo[3,2-c]pyridin-6-amine;
N-(2-(2-methoxypyrimidin-4-yl)-1H-pyrrolo[3,2-c]pyridin-6-yl)cyclopropanecarboxamide;
(1r,3r)-3-hydroxy-N-(2-(2-methoxypyrimidin-4-yl)-1-methyl-1H-pyrrolo[3,2-c]pyridin-6-yl)cyclobutanecarboxamide;
2-methyl-5-(2-(pyridin-4-yl)-1H-pyrrolo[3,2-c]pyridin-6-ylamino)isoindolin-1-one;
2-(oxazol-5-yl)-N-(3-(tetrahydro-2H-pyran-4-yl)phenyl)-1H-pyrrolo[3,2-c]pyridin-6-amine;
N-(2-(oxazol-5-yl)-1H-pyrrolo[3,2-c]pyridin-6-yl)cyclopropanecarboxamide;
N-(2-(2,6-difluoropyridin-4-yl)-1H-pyrrolo[3,2-c]pyridin-6-yl)cyclopropanecarboxamide;
2-(oxazol-5-yl)-N-(tetrahydro-2H-pyran-4-yl)-1H-pyrrolo[3,2-c]pyridin-6-amine;
N-(1-methyl-2-(1H-pyrazol-4-yl)-1H-pyrrolo[3,2-c]pyridin-6-yl)cyclopropanecarboxamide;
N-(2-(2-methyloxazol-5-yl)-1H-pyrrolo[3,2-c]pyridin-6-yl)cyclopropanecarboxamide;
N-(2-(2-cyanopyridin-4-yl)-1H-pyrrolo[3,2-c]pyridin-6-yl)cyclopropanecarboxamide;
(R)-2-(2-methylpyridin-4-yl)-N-(tetrahydrofuran-3-yl)-1H-pyrrolo[3,2-c]pyridin-6-amine;
2-(pyridin-4-yl)-N-(3-(tetrahydro-2H-pyran-4-yl)phenyl)-1H-pyrrolo[3,2-c]pyridin-6-amine;
2-(2-methylpyridin-4-yl)-N-(4-methyltetrahydro-2H-pyran-4-yl)-1H-pyrrolo[3,2-c]pyridin-6-amine;
N-(2-(2-ethoxypyrimidin-4-yl)-1H-pyrrolo[3,2-c]pyridin-6-yl)cyclopropanecarboxamide;
N-(2-(2-methylpyridin-4-yl)-1H-pyrrolo[3,2-c]pyridin-6-yl)bicyclo[4.1.0]heptane-7-carboxamide;
N-(2-(2-(trifluoromethyl)pyrimidin-4-yl)-1H-pyrrolo[3,2-c]pyridin-6-yl)cyclopropanecarboxamide;
N-(2-(pyridin-3-yl)-1H-pyrrolo[3,2-c]pyridin-6-yl)cyclopropanecarboxamide;
2-(6-methylpyrimidin-4-yl)-N-(tetrahydro-2H-pyran-4-yl)-1H-pyrrolo[3,2-c]pyridin-6-amine;
1-methyl-2-(6-methylpyrimidin-4-yl)-N-(tetrahydro-2H-pyran-4-yl)-1H-pyrrolo[3,2-c]pyridin-6-amine;
2-(2-methoxypyrimidin-4-yl)-N-(1-(tetrahydro-2H-pyran-4-yl)-1H-pyrazol-4-yl)-1H-pyrrolo[3,2-c]pyridin-6-amine;
N-(2-m-tolyl-1H-pyrrolo[3,2-c]pyridin-6-yl)cyclopropanecarboxamide;
N-(2-(5-methyl-1H-pyrazol-4-yl)-1H-pyrrolo[3,2-c]pyridin-6-yl)cyclopropanecarboxamide;
(S)-tetrahydrofuran-3-yl 2-(2-methylpyridin-4-yl)-1H-pyrrolo[3,2-c]pyridin-6-ylcarbamate;
1-methyl-N-(1-methyl-2-(2-(2,2,2-trifluoroethylamino)pyrimidin-4-yl)-1H-pyrrolo[3,2-c]pyridin-6-yl)-1H-pyrazole-4-carboxamide;
(1R,2R)—N-(2-(2-methoxypyrimidin-4-yl)-1H-pyrrolo[3,2-c]pyridin-6-yl)-2-methylcyclopropanecarboxamide;
N-(1-methyl-2-(6-methylpyrimidin-4-yl)-1H-pyrrolo[3,2-c]pyridin-6-yl)cyclopropanecarboxamide;
N-(2-(thiazol-5-yl)-1H-pyrrolo[3,2-c]pyridin-6-yl)cyclopropanecarboxamide;
N-(2-(6-methylpyrimidin-4-yl)-1H-pyrrolo[3,2-c]pyridin-6-yl)cyclopropanecarboxamide;
2-(2-methylpyrimidin-4-yl)-N-(tetrahydro-2H-pyran-4-yl)-1H-pyrrolo[3,2-c]pyridin-6-amine;
N-(2-(2-methylpyrimidin-4-yl)-1H-pyrrolo[3,2-c]pyridin-6-yl)cyclopropanecarboxamide;
N-(1-methyl-2-(1H-1,2,4-triazol-3-yl)-1H-pyrrolo[3,2-c]pyridin-6-yl)cyclopropanecarboxamide;
(S)-2-(2-methylpyridin-4-yl)-N-(tetrahydrofuran-3-yl)-1H-pyrrolo[3,2-c]pyridin-6-amine;
N-(2-(3,3-difluorocyclobutyl)-1H-pyrrolo[3,2-c]pyridin-6-yl)cyclopropanecarboxamide;
4-(6-(3-(tetrahydro-2H-pyran-4-yl)phenylamino)-1H-pyrrolo[3,2-c]pyridin-2-yl)picolinonitrile;
N-(1-(2-hydroxyethyl)-2-(6-methylpyrimidin-4-yl)-1H-pyrrolo[3,2-c]pyridin-6-yl)cyclopropanecarboxamide;
4-(6-(1-methyl-1H-pyrazol-4-ylamino)-1H-pyrrolo[3,2-c]pyridin-2-yl)picolinonitrile;
N-(1-methyl-2-(1-methyl-1H-1,2,4-triazol-3-yl)-1H-pyrrolo[3,2-c]pyridin-6-yl)cyclopropanecarboxamide;
N-(2-(2-ethoxypyrimidin-4-yl)-1H-pyrrolo[3,2-c]pyridin-6-yl)bicyclo[3.1.0]hexane-6-carboxamide;
1-methyl-2-(2-methylpyridin-4-yl)-N-(tetrahydro-2H-pyran-4-yl)-1H-pyrrolo[3,2-c]pyridin-6-amine;
N-(2-(1-methyl-1H-imidazol-4-yl)-1H-pyrrolo[3,2-c]pyridin-6-yl)cyclopropanecarboxamide;
N-(2-(2-cyclopropyloxazol-5-yl)-1H-pyrrolo[3,2-c]pyridin-6-yl)cyclopropanecarboxamide;
2-(2-methoxypyrimidin-4-yl)-N-(tetrahydro-2H-pyran-4-yl)-1H-pyrrolo[3,2-c]pyridin-6-amine;
(1r,3r)-3-hydroxy-N-(2-(2-methoxypyrimidin-4-yl)-1H-pyrrolo[3,2-c]pyridin-6-yl)cyclobutanecarboxamide;
1-methyl-N-(2-(oxazol-5-yl)-1H-pyrrolo[3,2-c]pyridin-6-yl)-1H-pyrazole-4-carboxamide;
N-(2-(1-methyl-1H-1,2,4-triazol-3-yl)-1H-pyrrolo[3,2-c]pyridin-6-yl)cyclopropanecarboxamide;
N-(3-(1H-1,2,4-triazol-3-yl)phenyl)-2-(2-methylpyridin-4-yl)-1H-pyrrolo[3,2-c]pyridin-6-amine;
N-(cyclopropylmethyl)-2-(2-methylpyridin-4-yl)-1H-pyrrolo[3,2-c]pyridin-6-amine;
N-(2-(pyridin-4-yl)-1H-pyrrolo[3,2-c]pyridin-6-yl)tetrahydro-2H-pyran-4-carboxamide;
N-(1-methyl-2-(6-methylpyridin-2-yl)-1H-pyrrolo[3,2-c]pyridin-6-yl)cyclopropanecarboxamide;
1-methyl-2-(1-methyl-1H-pyrazol-4-yl)-N-phenyl-1H-pyrrolo[3,2-c]pyridin-6-amine;
N-(tetrahydro-2H-pyran-4-yl)-2-(trifluoromethyl)-1H-pyrrolo[3,2-c]pyridin-6-amine; 2-(pyridin-3-yl)-N-(tetrahydro-2H-pyran-4-yl)-1H-pyrrolo[3,2-c]pyridin-6-amine;
N-(1-methyl-2-(4-methylpyridin-2-yl)-1H-pyrrolo[3,2-c]pyridin-6-yl)cyclopropanecarboxamide;
N-(1-methyl-2-(oxazol-5-yl)-1H-pyrrolo[3,2-c]pyridin-6-yl)cyclopropanecarboxamide;
N-(1-(2-aminoethyl)-2-(6-methylpyrimidin-4-yl)-1H-pyrrolo[3,2-c]pyridin-6-yl)cyclopropanecarboxamide;
N-(2-(trifluoromethyl)-1H-pyrrolo[3,2-c]pyridin-6-yl)cyclobutanecarboxamide;
N-(1-methyl-2-(trifluoromethyl)-1H-pyrrolo[3,2-c]pyridin-6-yl)cyclopropanecarboxamide;
N-(2-(pyridin-4-yl)-1H-pyrrolo[3,2-c]pyridin-6-yl)isobutyramide;
4-(6-(3-(4H-1,2,4-triazol-3-yl)phenylamino)-1H-pyrrolo[3,2-c]pyridin-2-yl)picolinonitrile;
N-(1-(2-hydroxyethyl)-2-(2-(2,2,2-trifluoroethylamino)pyrimidin-4-yl)-1H-pyrrolo[3,2-c]pyridin-6-yl)-1-methyl-1H-pyrazole-4-carboxamide;
2-(2-methylpyridin-4-yl)-1H-pyrrolo[3,2-c]pyridin-6-amine;
N-(2-(1H-imidazol-5-yl)-1H-pyrrolo[3,2-c]pyridin-6-yl)-1-methyl-1H-pyrazole-4-carboxamide;
N-(1-methyl-2-(2-methylpyridin-4-yl)-1H-pyrrolo[3,2-c]pyridin-6-yl)cyclopropanecarboxamide;
1-cyclopentyl-N-(2-(2-methylpyridin-4-yl)-1H-pyrrolo[3,2-c]pyridin-6-yl)-1H-pyrazole-4-carboxamide;
N-(2-(2-((1r,4r)-4-hydroxycyclohexyloxy)pyrimidin-4-yl)-1H-pyrrolo[3,2-c]pyridin-6-yl)-1-methyl-1H-pyrazole-4-carboxamide;
N-methyl-N-(2-(2-methylpyridin-4-yl)-1H-pyrrolo[3,2-c]pyridin-6-yl)cyclopropanecarboxamide;
N-(2-(2-methoxypyrimidin-4-yl)-1-methyl-1H-pyrrolo[3,2-c]pyridin-6-yl)-2-(oxetan-3-yl)acetamide;
N-(2-cyclopropyl-1H-pyrrolo[3,2-c]pyridin-6-yl)-1-methyl-1H-pyrazole-4-carboxamide;
N-(2-(3-hydroxypropyl)-1H-pyrrolo[3,2-c]pyridin-6-yl)-1-methyl-1H-pyrazole-4-carboxamide;
1-methyl-N-(2-(1-methyl-1H-imidazol-5-yl)-1H-pyrrolo[3,2-c]pyridin-6-yl)-1H-pyrazole-4-carboxamide;
2-(oxetan-3-yl)-N-(2-(2-(trifluoromethyl)pyrimidin-4-yl)-1H-pyrrolo[3,2-c]pyridin-6-yl)acetamide;
(1r,3r)-3-hydroxy-N-(2-(2-(trifluoromethyl)pyrimidin-4-yl)-1H-pyrrolo[3,2-c]pyridin-6-yl)cyclobutanecarboxamide;
N-(1-(2-hydroxyethyl)-2-(2-methylpyridin-4-yl)-1H-pyrrolo[3,2-c]pyridin-6-yl)cyclopropanecarboxamide;
N-(2-(tetrahydro-2H-pyran-4-yl)-1H-pyrrolo[3,2-c]pyridin-6-yl)cyclopropanecarboxamide;
1-methyl-N-(2-(2-methylpyridin-4-yl)-1H-pyrrolo[3,2-c]pyridin-6-yl)cyclopropanecarboxamide;
N-(2-(2-methylpyridin-4-yl)-1H-pyrrolo[3,2-c]pyridin-6-yl)cyclopropanecarboximidamide;
N-(2-(2-methylpyridin-4-yl)-1H-pyrrolo[3,2-c]pyridin-6-yl)cyclopropanesulfonamide;
N-(2-(6-methylpyrimidin-4-yl)-1H-pyrrolo[3,2-c]pyridin-6-yl)cyclobutanecarboxamide;
N-(2-(2-methylpyrimidin-4-yl)-1H-pyrrolo[3,2-c]pyridin-6-yl)cyclobutanecarboxamide;
N-(1-methyl-2-(2-methylpyrimidin-4-yl)-1H-pyrrolo[3,2-c]pyridin-6-yl)cyclopropanecarboxamide;
N-(2-(2-methylthiazol-5-yl)-1H-pyrrolo[3,2-c]pyridin-6-yl)cyclopropanecarboxamide;
N-(1-methyl-2-(1-methyl-1H-pyrazol-4-yl)-1H-pyrrolo[3,2-c]pyridin-6-yl)cyclopropanecarboxamide;
N-(2-(2-(trifluoromethyl)thiazol-5-yl)-1H-pyrrolo[3,2-c]pyridin-6-yl)cyclopropanecarboxamide;
N-(2-(4-(trifluoromethyl)thiazol-2-yl)-1H-pyrrolo[3,2-c]pyridin-6-yl)cyclopropanecarboxamide;
N-(2-(trifluoromethyl)-1H-pyrrolo[3,2-c]pyridin-6-yl)tetrahydro-2H-pyran-4-carboxamide;
N-(2-(trifluoromethyl)-1H-pyrrolo[3,2-c]pyridin-6-yl)cyclopropanesulfonamide;
methyl 2-(trifluoromethyl)-1H-pyrrolo[3,2-c]pyridin-6-ylcarbamate;
1,1-dimethyl-3-(2-(trifluoromethyl)-1H-pyrrolo[3,2-c]pyridin-6-yl)urea;
1-methyl-N-(2-(trifluoromethyl)-1H-pyrrolo[3,2-c]pyridin-6-yl)piperidine-4-carboxamide;
N-(2-(2-(trifluoromethyl)oxazol-5-yl)-1H-pyrrolo[3,2-c]pyridin-6-yl)cyclopropanecarboxamide;
N-(2-(trifluoromethyl)-1H-pyrrolo[3,2-c]pyridin-6-yl)cyclopentanecarboxamide;
N-(1-methylpiperidin-4-yl)-2-(2-methylpyridin-4-yl)-1H-pyrrolo[3,2-c]pyridin-6-amine;
2-(2-methylpyridin-4-yl)-N-(oxetan-3-yl)-1H-pyrrolo[3,2-c]pyridin-6-amine;
N-methyl-2-(2-methylpyridin-4-yl)-N-(tetrahydro-2H-pyran-4-yl)-1H-pyrrolo[3,2-c]pyridin-6-amine;
N-(3-methyl-1H-pyrrolo[3,2-c]pyridin-6-yl)cyclopropanecarboxamide;
N-(1-(2-hydroxyethyl)-2-(trifluoromethyl)-1H-pyrrolo[3,2-c]pyridin-6-yl)cyclopropanecarboxamide;
1-methyl-N-(1-methylpiperidin-4-yl)-2-(6-methylpyrimidin-4-yl)-1H-pyrrolo[3,2-c]pyridin-6-amine;
N-(1-methyl-2-(6-methylpyrimidin-4-yl)-1H-pyrrolo[3,2-c]pyridin-6-yl)isobutyramide;
N-(1-methyl-2-(6-methylpyrimidin-4-yl)-1H-pyrrolo[3,2-c]pyridin-6-yl)acetamide;
1-methyl-2-(6-methylpyrimidin-4-yl)-N-(piperidin-4-yl)-1H-pyrrolo[3,2-c]pyridin-6-amine;
2-cyclobutyl-N-(tetrahydro-2H-pyran-4-yl)-1H-pyrrolo[3,2-c]pyridin-6-amine;
N-(1-ethyl-2-(6-methylpyrimidin-4-yl)-1H-pyrrolo[3,2-c]pyridin-6-yl)cyclopropanecarboxamide;
2-(1-methyl-1H-imidazol-4-yl)-N-(tetrahydro-2H-pyran-4-yl)-1H-pyrrolo[3,2-c]pyridin-6-amine;
N-(1-(2-(dimethylamino)ethyl)-2-(6-methylpyrimidin-4-yl)-1H-pyrrolo[3,2-c]pyridin-6-yl)cyclopropanecarboxamide;
1-methyl-2-(6-methylpyrimidin-4-yl)-1H-pyrrolo[3,2-c]pyridin-6-amine;
N,1-dimethyl-2-(6-methylpyrimidin-4-yl)-1H-pyrrolo[3,2-c]pyridin-6-amine;
2-(dimethylamino)-N-(1-methyl-2-(6-methylpyrimidin-4-yl)-1H-pyrrolo[3,2-c]pyridin-6-yl)acetamide;
N-(1-methyl-2-(6-methylpyrimidin-4-yl)-1H-pyrrolo[3,2-c]pyridin-6-yl)-2-(pyrrolidin-1-yl)acetamide;
N-(1-methyl-2-(pyridin-2-yl)-1H-pyrrolo[3,2-c]pyridin-6-yl)cyclopropanecarboxamide;
N-(1-methyl-2-(6-(trifluoromethyl)pyrimidin-4-yl)-1H-pyrrolo[3,2-c]pyridin-6-yl)cyclopropanecarboxamide;
N-(2-(2-(cyclopropylmethylamino)pyrimidin-4-yl)-1-methyl-1H-pyrrolo[3,2-c]pyridin-6-yl)-1-isopropyl-1H-pyrazole-4-carboxamide;
N-(2-(2-(cyclopropylamino)pyrimidin-4-yl)-1-methyl-1H-pyrrolo[3,2-c]pyridin-6-yl)-1-isopropyl-1H-pyrazole-4-carboxamide;
1-methyl-N-(1-methyl-2-(2-(3,3,3-trifluoropropylamino)pyrimidin-4-yl)-1H-pyrrolo[3,2-c]pyridin-6-yl)-1H-pyrazole-4-carboxamide;
3-methyl-N-(2-(pyridin-4-yl)-1H-pyrrolo[3,2-c]pyridin-6-yl)butanamide;
(R)—N-(2-(pyridin-4-yl)-1H-pyrrolo[3,2-c]pyridin-6-yl)tetrahydrofuran-3-carboxamide;
(S)-1,3-dimethyl-N-(2-(2-(1,1,1-trifluoropropan-2-yloxy)pyrimidin-4-yl)-1H-pyrrolo[3,2-c]pyridin-6-yl)-1H-pyrazole-4-carboxamide;
N-(2-(pyridin-4-yl)-1H-pyrrolo[3,2-c]pyridin-6-yl)cyclopentanecarboxamide;
1-methyl-2-(pyridin-4-yl)-N-(1-(tetrahydro-2H-pyran-4-yl)-1H-pyrazol-4-yl)-1H-pyrrolo[3,2-c]pyridin-6-amine;
N-(2-(2-ethoxypyrimidin-4-yl)-1H-pyrrolo[3,2-c]pyridin-6-yl)-2-(oxetan-3-yl)acetamide;
N-(3-methoxy-5-(1H-1,2,4-triazol-1-yl)phenyl)-2-(pyridin-4-yl)-1H-pyrrolo[3,2-c]pyridin-6-amine;
N-(2-(2-(1,1-difluoropropan-2-yloxy)pyrimidin-4-yl)-1H-pyrrolo[3,2-c]pyridin-6-yl)-1-methyl-1H-pyrazole-4-carboxamide;
(R)—N-(1-methyl-2-(2-(2,2,2-trifluoroethylamino)pyrimidin-4-yl)-1H-pyrrolo[3,2-c]pyridin-6-yl)-1-(tetrahydrofuran-3-yl)-1H-pyrazole-4-carboxamide;
N-(2-(pyridin-4-yl)-1H-pyrrolo[3,2-c]pyridin-6-yl)-2-(tetrahydro-2H-pyran-4-yl)acetamide;
N-(2-(2-(2,2-difluoroethylamino)pyrimidin-4-yl)-1-methyl-1H-pyrrolo[3,2-c]pyridin-6-yl)-1-methyl-1H-pyrazole-4-carboxamide;
6-(3-(1H-1,2,4-triazol-3-yl)phenylamino)-1H-pyrrolo[3,2-c]pyridine-2-carbonitrile;
N-(2-(2-(2-fluoroethoxy)pyrimidin-4-yl)-1H-pyrrolo[3,2-c]pyridin-6-yl)-1-isopropyl-1H-pyrazole-4-carboxamide;
2-(oxazol-5-yl)-N-(1-(tetrahydro-2H-pyran-4-yl)-1H-pyrazol-4-yl)-1H-pyrrolo[3,2-c]pyridin-6-amine;
6-(3-methoxy-5-(tetrahydro-2H-pyran-4-yl)phenylamino)-1H-pyrrolo[3,2-c]pyridine-2-carbonitrile;
1-isopropyl-N-(2-(2-(1,1,1-trifluoropropan-2-yloxy)pyrimidin-4-yl)-1H-pyrrolo[3,2-c]pyridin-6-yl)-1H-pyrazole-4-carboxamide;
N-(3-(1H-1,2,4-triazol-3-yl)phenyl)-2-(2-amino-6-methylpyridin-4-yl)-1H-pyrrolo[3,2-c]pyridin-6-amine;
6-(1-(tetrahydro-2H-pyran-4-yl)-1H-pyrazol-4-ylamino)-1H-pyrrolo[3,2-c]pyridine-2-carbonitrile;
(R)-tetrahydrofuran-3-yl 2-(2-methylpyridin-4-yl)-1H-pyrrolo[3,2-c]pyridin-6-ylcarbamate;
(1R,2R)—N-(2-(2-ethoxypyrimidin-4-yl)-1H-pyrrolo[3,2-c]pyridin-6-yl)-2-methylcyclopropanecarboxamide;
N-(2-(pyridin-4-yl)-1H-pyrrolo[3,2-c]pyridin-6-yl)-1-(tetrahydro-2H-pyran-4-yl)-1H-pyrazole-4-carboxamide;
N-(2-(2-methylpyridin-4-yl)-1H-pyrrolo[3,2-c]pyridin-6-yl)tetrahydro-2H-pyran-3-carboxamide;
(S)—N-(2-(2-methoxypyrimidin-4-yl)-1H-pyrrolo[3,2-c]pyridin-6-yl)-1-((tetrahydrofuran-3-yl)methyl)-1H-pyrazole-4-carboxamide;
(S)—N-(2-(pyridin-4-yl)-1H-pyrrolo[3,2-c]pyridin-6-yl)tetrahydrofuran-3-carboxamide;
N-(2-(2-ethoxypyrimidin-4-yl)-1H-pyrrolo[3,2-c]pyridin-6-yl)bicyclo[4.1.0]heptane-7-carboxamide;
N-(2-(2-(cyclopropylmethylamino)pyrimidin-4-yl)-1-methyl-1H-pyrrolo[3,2-c]pyridin-6-yl)-1-methyl-1H-pyrazole-4-carboxamide;
N-(2-(2-methylpyridin-4-yl)-1H-pyrrolo[3,2-c]pyridin-6-yl)isobutyramide;
(1r,3r)-3-methoxy-N-(2-(2-methylpyridin-4-yl)-1H-pyrrolo[3,2-c]pyridin-6-yl)cyclobutanecarboxamide;
1-isopropyl-N-(2-(pyridin-4-yl)-1H-pyrrolo[3,2-c]pyridin-6-yl)-1H-pyrazole-4-carboxamide;
(1s,3s)-3-hydroxy-N-(2-(2-methoxypyrimidin-4-yl)-1-methyl-1H-pyrrolo[3,2-c]pyridin-6-yl)cyclobutanecarboxamide;
6-(1-methyl-1H-pyrazol-4-ylamino)-1H-pyrrolo[3,2-c]pyridine-2-carbonitrile;
N-(2-(2-methylpyridin-4-yl)-1H-pyrrolo[3,2-c]pyridin-6-yl)-2-(oxetan-3-yl)acetamide;
1,5-dimethyl-N-(2-(pyridin-4-yl)-1H-pyrrolo[3,2-c]pyridin-6-yl)-1H-pyrazole-4-carboxamide;
(1s,3s)-3-hydroxy-N-(2-(2-methoxypyrimidin-4-yl)-1H-pyrrolo[3,2-c]pyridin-6-yl)cyclobutanecarboxamide;
N-(1-isopropyl-5-methyl-1H-pyrazol-3-yl)-2-(pyridin-4-yl)-1H-pyrrolo[3,2-c]pyridin-6-amine;
N-(2-(2-(2,2-difluoroethylamino)pyrimidin-4-yl)-1-methyl-1H-pyrrolo[3,2-c]pyridin-6-yl)-1-isopropyl-1H-pyrazole-4-carboxamide;
cyclopentyl 2-(2-methylpyridin-4-yl)-1H-pyrrolo[3,2-c]pyridin-6-ylcarbamate;
(S)—N-(2-(2-(3-fluoropropoxy)pyrimidin-4-yl)-1H-pyrrolo[3,2-c]pyridin-6-yl)-1-((tetrahydrofuran-2-yl)methyl)-1H-pyrazole-4-carboxamide;
1-isopropyl-N-(2-(2-(2,2,2-trifluoroethylamino)pyrimidin-4-yl)-1H-pyrrolo[3,2-c]pyridin-6-yl)-1H-pyrazole-4-carboxamide;
N-(2-(2-(2,2-difluoroethoxy)pyrimidin-4-yl)-1H-pyrrolo[3,2-c]pyridin-6-yl)-1-isopropyl-1H-pyrazole-4-carboxamide;
methyl 2-(2-methylpyridin-4-yl)-1H-pyrrolo[3,2-c]pyridin-6-ylcarbamate;
1-isopropyl-N-(2-(2-(3,3,3-trifluoropropoxy)pyrimidin-4-yl)-1H-pyrrolo[3,2-c]pyridin-6-yl)-1H-pyrazole-4-carboxamide;
N-(2-(2-methoxypyrimidin-4-yl)-1H-pyrrolo[3,2-c]pyridin-6-yl)bicyclo[3.1.0]hexane-6-carboxamide;
1-isopropyl-N-(2-(2-methoxypyrimidin-4-yl)-1H-pyrrolo[3,2-c]pyridin-6-yl)-1H-pyrazole-4-carboxamide;
1-isopropyl-N-(1-methyl-2-(2-(3,3,3-trifluoropropylamino)pyrimidin-4-yl)-1H-pyrrolo[3,2-c]pyridin-6-yl)-1H-pyrazole-4-carboxamide;
(S)—N-(2-(2-methoxypyrimidin-4-yl)-1H-pyrrolo[3,2-c]pyridin-6-yl)-5-methyl-1-((tetrahydrofuran-3-yl)methyl)-1H-pyrazole-4-carboxamide;
N-(2-cyano-1H-pyrrolo[3,2-c]pyridin-6-yl)cyclopropanecarboxamide;
N-(2-(pyridin-4-yl)-1H-pyrrolo[3,2-c]pyridin-6-yl)acetamide;
(S)—N-(2-(2-methylpyridin-4-yl)-1H-pyrrolo[3,2-c]pyridin-6-yl)tetrahydrofuran-2-carboxamide;
1-cyclopentyl-N-(2-(2-methoxypyrimidin-4-yl)-1H-pyrrolo[3,2-c]pyridin-6-yl)-1H-pyrazole-4-carboxamide;
(R)—N-(2-(2-methoxypyrimidin-4-yl)-1H-pyrrolo[3,2-c]pyridin-6-yl)-1-((tetrahydrofuran-3-yl)methyl)-1H-pyrazole-4-carboxamide;
1-methyl-N-(2-(2-methylpyrimidin-4-yl)-1H-pyrrolo[3,2-c]pyridin-6-yl)-1H-pyrazole-4-carboxamide;
1-isopropyl-N-(2-(2-(2,2,2-trifluoroethoxy)pyrimidin-4-yl)-1H-pyrrolo[3,2-c]pyridin-6-yl)-1H-pyrazole-4-carboxamide;
(1r,3r)-3-hydroxy-N-(2-(2-methylpyridin-4-yl)-1H-pyrrolo[3,2-c]pyridin-6-yl)cyclobutanecarboxamide;
N-(2-(2-methoxypyrimidin-4-yl)-1-methyl-1H-pyrrolo[3,2-c]pyridin-6-yl)-4-(tetrahydro-2H-pyran-4-yloxy)benzamide;
N-phenyl-2-(pyridin-3-yl)-1H-pyrrolo[3,2-c]pyridin-6-amine;
N-methyl-4-(2-(2-methylpyridin-4-yl)-1H-pyrrolo[3,2-c]pyridin-6-ylamino)benzamide;
(1s,3s)-3-methoxy-N-(2-(2-methylpyridin-4-yl)-1H-pyrrolo[3,2-c]pyridin-6-yl)cyclobutanecarboxamide;
N-(1-(2-methoxyethyl)-1H-pyrazol-4-yl)-2-(pyridin-4-yl)-1H-pyrrolo[3,2-c]pyridin-6-amine;
1-methyl-2-(1-methyl-1H-pyrazol-4-yl)-N-(4-(methylthio)phenyl)-1H-pyrrolo[3,2-c]pyridin-6-amine;
(1s,3s)-3-hydroxy-N-(2-(2-methylpyridin-4-yl)-1H-pyrrolo[3,2-c]pyridin-6-yl)cyclobutanecarboxamide;
1-methyl-N-(2-(pyridin-4-yl)-1H-pyrrolo[3,2-c]pyridin-6-yl)-1H-indazol-6-amine;
(S)—N-(2-(2-methoxypyrimidin-4-yl)-1H-pyrrolo[3,2-c]pyridin-6-yl)-5-methyl-1-((tetrahydrofuran-2-yl)methyl)-1H-pyrazole-4-carboxamide;
N-(3-methoxyphenyl)-2-(1-methyl-1H-pyrazol-4-yl)-1H-pyrrolo[3,2-c]pyridin-6-amine;
N-(2-(pyridin-3-yl)-1H-pyrrolo[3,2-c]pyridin-6-yl)acetamide;
N-(2-(2-(trifluoromethyl)pyrimidin-4-yl)-1H-pyrrolo[3,2-c]pyridin-6-yl)bicyclo[3.1.0]hexane-6-carboxamide;
1-methyl-N-(1-methyl-2-(2-(trifluoromethyl)pyrimidin-4-yl)-1H-pyrrolo[3,2-c]pyridin-6-yl)-1H-pyrazole-4-carboxamide;
N-(2-(1H-1,2,4-triazol-3-yl)-1H-pyrrolo[3,2-c]pyridin-6-yl)cyclopropanecarboxamide;
3-hydroxy-3-methyl-N-(2-(2-methylpyridin-4-yl)-1H-pyrrolo[3,2-c]pyridin-6-yl)cyclobutanecarboxamide;
N-(4-methoxyphenyl)-1-methyl-2-(1-methyl-1H-pyrazol-4-yl)-1H-pyrrolo[3,2-c]pyridin-6-amine;
N-(3-methoxyphenyl)-2-(1-methyl-1H-imidazol-5-yl)-1H-pyrrolo[3,2-c]pyridin-6-amine;
1-cyclobutyl-N-(2-(2-methylpyridin-4-yl)-1H-pyrrolo[3,2-c]pyridin-6-yl)-1H-pyrazole-4-carboxamide;
4-methoxycyclohexyl 2-(2-methylpyridin-4-yl)-1H-pyrrolo[3,2-c]pyridin-6-ylcarbamate;
2-cyclopentyl-N-(2-(pyridin-4-yl)-1H-pyrrolo[3,2-c]pyridin-6-yl)acetamide;
6-(3-(tetrahydro-2H-pyran-4-yl)-5-(1H-1,2,4-triazol-3-yl)phenylamino)-1H-pyrrolo[3,2-c]pyridine-2-carbonitrile;
1-cyclopropyl-3-(2-(2-methylpyridin-4-yl)-1H-pyrrolo[3,2-c]pyridin-6-yl)urea;
N-(2-(1-methyl-1H-pyrazol-4-yl)-1H-pyrrolo[3,2-c]pyridin-6-yl)cyclopropanecarboxamide;
N-(2-(2-methoxypyrimidin-4-yl)-1-methyl-1H-pyrrolo[3,2-c]pyridin-6-yl)bicyclo[4.1.0]heptane-7-carboxamide;
(R)—N-(2-(2-methylpyridin-4-yl)-1H-pyrrolo[3,2-c]pyridin-6-yl)tetrahydrofuran-2-carboxamide;
1-methyl-N-(2-(2-(3,3,3-trifluoropropoxy)pyrimidin-4-yl)-1H-pyrrolo[3,2-c]pyridin-6-yl)-1H-pyrazole-4-carboxamide;
1-methyl-3-(2-(2-methylpyridin-4-yl)-1H-pyrrolo[3,2-c]pyridin-6-yl)urea;
2-(4-(6-(3-(1H-1,2,4-triazol-3-yl)phenylamino)-1H-pyrrolo[3,2-c]pyridin-2-yl)pyridin-2-yl)propan-2-ol;
1-((1r,4r)-4-hydroxycyclohexyl)-N-(2-(2-(3,3,3-trifluoropropoxy)pyrimidin-4-yl)-1H-pyrrolo[3,2-c]pyridin-6-yl)-1H-pyrazole-4-carboxamide;
N-(2-(pyridin-4-yl)-1H-pyrrolo[3,2-c]pyridin-6-yl)cyclohexanecarboxamide;
3-(difluoromethyl)-1-methyl-N-(2-(2-methylpyridin-4-yl)-1H-pyrrolo[3,2-c]pyridin-6-yl)-1H-pyrazole-5-carboxamide;
1-((1r,4r)-4-hydroxycyclohexyl)-N-(2-(2-methoxypyrimidin-4-yl)-1H-pyrrolo[3,2-c]pyridin-6-yl)-1H-pyrazole-4-carboxamide;
N-(2-(2-methoxypyrimidin-4-yl)-1H-pyrrolo[3,2-c]pyridin-6-yl)-1-methyl-1H-indazole-4-carboxamide;
3,5-dimethyl-N-(2-(2-methylpyridin-4-yl)-1H-pyrrolo[3,2-c]pyridin-6-yl)isoxazole-4-carboxamide;
2-(1-methyl-1H-pyrazol-4-yl)-N-(4-(trifluoromethyl)phenyl)-1H-pyrrolo[3,2-c]pyridin-6-amine;
1-methyl-2-(1-methyl-1H-pyrazol-4-yl)-N-(tetrahydro-2H-pyran-4-yl)-1H-pyrrolo[3,2-c]pyridin-6-amine;
1-methyl-N-(2-(2-methylpyridin-4-yl)-1H-pyrrolo[3,2-c]pyridin-6-yl)-1H-pyrazole-4-carboxamide;
N-(2-(2-(2-fluoroethoxy)pyrimidin-4-yl)-1H-pyrrolo[3,2-c]pyridin-6-yl)-1-(tetrahydro-2H-pyran-4-yl)-1H-pyrazole-4-carboxamide;
1,3-dimethyl-N-(2-(2-(2,2,2-trifluoroethoxy)pyrimidin-4-yl)-1H-pyrrolo[3,2-c]pyridin-6-yl)-1H-pyrazole-4-carboxamide;
N-(2-(2-methoxypyrimidin-4-yl)-1H-pyrrolo[3,2-c]pyridin-6-yl)-2-(oxetan-3-yl)acetamide;
N-((1s,4s)-4-methoxycyclohexyl)-2-(pyridin-3-yl)-1H-pyrrolo[3,2-c]pyridin-6-amine;
N-(4-fluorophenyl)-2-(1-methyl-1H-imidazol-5-yl)-1H-pyrrolo[3,2-c]pyridin-6-amine;
N-(2-(2-ethoxypyrimidin-4-yl)-1H-pyrrolo[3,2-c]pyridin-6-yl)-1-(2-hydroxyethyl)-5-methyl-1H-pyrazole-4-carboxamide;
(R)—N-(2-(2-methoxypyrimidin-4-yl)-1H-pyrrolo[3,2-c]pyridin-6-yl)-1-((tetrahydrofuran-2-yl)methyl)-1H-pyrazole-4-carboxamide;
1-isopropyl-N-(2-(2-(methyl(2,2,2-trifluoroethyl)amino)pyrimidin-4-yl)-1H-pyrrolo[3,2-c]pyridin-6-yl)-1H-pyrazole-4-carboxamide;
N-(2-(2-ethoxypyrimidin-4-yl)-1H-pyrrolo[3,2-c]pyridin-6-yl)-1-(2-hydroxyethyl)-3-methyl-1H-pyrazole-4-carboxamide;
(S)—N-(2-(2-(2,2-difluoroethoxy)pyrimidin-4-yl)-1H-pyrrolo[3,2-c]pyridin-6-yl)-1-((tetrahydrofuran-2-yl)methyl)-1H-pyrazole-4-carboxamide;
N-(4-fluorophenyl)-1-methyl-2-(1-methyl-1H-pyrazol-4-yl)-1H-pyrrolo[3,2-c]pyridin-6-amine;
N-(2-(2-methylpyridin-4-yl)-1H-pyrrolo[3,2-c]pyridin-6-yl)-1-(tetrahydrofuran-3-yl)-1H-pyrazole-4-carboxamide;
N-(2-(2-methylpyridin-4-yl)-1H-pyrrolo[3,2-c]pyridin-6-yl)-1H-indazole-7-carboxamide;
1-methyl-N-(2-(pyridin-4-yl)-1H-pyrrolo[3,2-c]pyridin-6-yl)-1H-pyrazole-3-carboxamide;
N-(2-(2-cyanopyridin-4-yl)-1H-pyrrolo[3,2-c]pyridin-6-yl)-1-methyl-1H-pyrazole-4-carboxamide;
1,3-dimethyl-N-(1-methyl-2-(2-(2,2,2-trifluoroethylamino)pyrimidin-4-yl)-1H-pyrrolo[3,2-c]pyridin-6-yl)-1H-pyrazole-4-carboxamide;
2-(1-isopropyl-1H-pyrazol-4-yl)-1-methyl-N-(tetrahydro-2H-pyran-4-yl)-1H-pyrrolo[3,2-c]pyridin-6-amine;
1-ethyl-3-methyl-N-(2-(2-(2,2,2-trifluoroethoxy)pyrimidin-4-yl)-1H-pyrrolo[3,2-c]pyridin-6-yl)-1H-pyrazole-4-carboxamide;
N-(2-(2-ethoxypyrimidin-4-yl)-1H-pyrrolo[3,2-c]pyridin-6-yl)-1H-indazole-7-carboxamide;
(1r,3r)-N-(2-(2-ethoxypyrimidin-4-yl)-1H-pyrrolo[3,2-c]pyridin-6-yl)-3-hydroxy-3-(trifluoromethyl)cyclobutanecarboxamide;
N-(2-cyano-1-methyl-1H-pyrrolo[3,2-c]pyridin-6-yl)acetamide;
(R)—N-(2-(2-methoxypyrimidin-4-yl)-1H-pyrrolo[3,2-c]pyridin-6-yl)-3-methyl-1-((tetrahydrofuran-3-yl)methyl)-1H-pyrazole-4-carboxamide;
(1s,3s)-3-hydroxy-N-(2-(2-(trifluoromethyl)pyrimidin-4-yl)-1H-pyrrolo[3,2-c]pyridin-6-yl)cyclobutanecarboxamide;
N-(2-(6-fluoropyridin-3-yl)-1H-pyrrolo[3,2-c]pyridin-6-yl)cyclopropanecarboxamide;
N-(2-(2-ethoxypyrimidin-4-yl)-1H-pyrrolo[3,2-c]pyridin-6-yl)-3-methoxy-4-(2-methoxyethoxy)benzamide;
N-(2-(2-methoxypyrimidin-4-yl)-1H-pyrrolo[3,2-c]pyridin-6-yl)-1,3-dimethyl-1H-pyrazole-4-carboxamide;
(1s,4s)-4-(2-(pyridin-3-yl)-1H-pyrrolo[3,2-c]pyridin-6-ylamino)cyclohexanol;
3-methoxy-4-(2-methoxyethoxy)-N-(2-(2-methoxypyrimidin-4-yl)-1H-pyrrolo[3,2-c]pyridin-6-yl)benzamide;
(1r,3r)-N-(2-(2-ethoxypyrimidin-4-yl)-1H-pyrrolo[3,2-c]pyridin-6-yl)-3-hydroxycyclobutanecarboxamide;
1-methyl-N-(2-(6-(trifluoromethyl)pyrimidin-4-yl)-1H-pyrrolo[3,2-c]pyridin-6-yl)-1H-pyrazole-4-carboxamide;
N-(2-(1-methylpiperidin-4-yl)-1H-pyrrolo[3,2-c]pyridin-6-yl)cyclopropanecarboxamide;
N-(2-(2-methoxypyrimidin-4-yl)-1-methyl-1H-pyrrolo[3,2-c]pyridin-6-yl)-1-methyl-1H-pyrazole-4-carboxamide;
N,1-dimethyl-N-(2-(2-(2,2,2-trifluoroethylamino)pyrimidin-4-yl)-1H-pyrrolo[3,2-c]pyridin-6-yl)-1H-pyrazole-4-carboxamide;
N-(4-methoxy-2-methylphenyl)-1-methyl-2-(1-methyl-1H-pyrazol-4-yl)-1H-pyrrolo[3,2-c]pyridin-6-amine;
1-methyl-N-(2-(tetrahydro-2H-pyran-4-yl)-1H-pyrrolo[3,2-c]pyridin-6-yl)-1H-pyrazole-4-carboxamide;
N-(2-(methoxymethyl)-1H-pyrrolo[3,2-c]pyridin-6-yl)-1-methyl-1H-pyrazole-4-carboxamide;
N-(2-(2-methylpyridin-4-yl)-1H-pyrrolo[3,2-c]pyridin-6-yl)pyrrolidine-1-carboxamide;
(1S,2S)-2-methyl-N-(2-(2-(trifluoromethyl)pyrimidin-4-yl)-1H-pyrrolo[3,2-c]pyridin-6-yl)cyclopropanecarboxamide;
(R)-2-hydroxy-N-(2-(2-methylpyridin-4-yl)-1H-pyrrolo[3,2-c]pyridin-6-yl)propanamide;
N-(2-(1H-1,2,4-triazol-3-yl)-1H-pyrrolo[3,2-c]pyridin-6-yl)-1-methyl-1H-pyrazole-4-carboxamide;
N-(1-methyl-2-phenyl-1H-pyrrolo[3,2-c]pyridin-6-yl)tetrahydro-2H-pyran-4-carboxamide;
1-methyl-2-(pyridin-3-yl)-N-(1-(tetrahydro-2H-pyran-4-yl)-1H-pyrazol-4-yl)-1H-pyrrolo[3,2-c]pyridin-6-amine;
1-methyl-N-(2-(2-methyloxazol-5-yl)-1H-pyrrolo[3,2-c]pyridin-6-yl)-1H-pyrazole-4-carboxamide;
N-(2-(5-fluoropyridin-2-yl)-1H-pyrrolo[3,2-c]pyridin-6-yl)cyclopropanecarboxamide;
N-(2-(2-methoxypyrimidin-4-yl)-1H-pyrrolo[3,2-c]pyridin-6-yl)-1H-indazole-4-carboxamide;
N-(2-(2-ethoxypyrimidin-4-yl)-1H-pyrrolo[3,2-c]pyridin-6-yl)-1-((1r,3r)-3-hydroxycyclobutyl)-1H-pyrazole-4-carboxamide;
N,2-diphenyl-1H-pyrrolo[3,2-c]pyridin-6-amine;
N-(3-(1H-1,2,4-triazol-3-yl)phenyl)-2-(pyridin-3-yl)-1H-pyrrolo[3,2-c]pyridin-6-amine;
N-isobutyl-2-(pyridin-3-yl)-1H-pyrrolo[3,2-c]pyridin-6-amine;
N-((1r,4r)-4-methoxycyclohexyl)-2-(pyridin-3-yl)-1H-pyrrolo[3,2-c]pyridin-6-amine;
N-(3-(1H-1,2,4-triazol-3-yl)phenyl)-1-methyl-2-(pyridin-3-yl)-1H-pyrrolo[3,2-c]pyridin-6-amine;
N-(2-(pyridin-3-yl)-1H-pyrrolo[3,2-c]pyridin-6-yl)cyclopentanecarboxamide;
N-(2-(pyridin-4-yl)-1H-pyrrolo[3,2-c]pyridin-6-yl)benzamide;
1,3-dimethyl-N-(2-(pyridin-4-yl)-1H-pyrrolo[3,2-c]pyridin-6-yl)-1H-pyrazole-4-carboxamide;
N-(2-cyano-1-methyl-1H-pyrrolo[3,2-c]pyridin-6-yl)-1-methyl-1H-pyrazole-4-carboxamide;
N-(2-cyano-1H-pyrrolo[3,2-c]pyridin-6-yl)-1-methyl-1H-pyrazole-4-carboxamide;
1,4-dimethyl-N-(2-(pyridin-4-yl)-1H-pyrrolo[3,2-c]pyridin-6-yl)-1H-pyrazole-3-carboxamide;
1,5-dimethyl-N-(2-(pyridin-4-yl)-1H-pyrrolo[3,2-c]pyridin-6-yl)-1H-pyrazole-3-carboxamide;
N-(2-(3-fluoro-2-methylpyridin-4-yl)-1H-pyrrolo[3,2-c]pyridin-6-yl)-1-methyl-1H-pyrazole-4-carboxamide;
1-methyl-N-(2-(2-(trifluoromethyl)pyridin-4-yl)-1H-pyrrolo[3,2-c]pyridin-6-yl)-1H-pyrazole-4-carboxamide;
1-methyl-N-(2-methyl-1H-pyrrolo[3,2-c]pyridin-6-yl)-1H-pyrazole-4-carboxamide;
N-(2-(2-methoxypyridin-4-yl)-1H-pyrrolo[3,2-c]pyridin-6-yl)-1-methyl-1H-pyrazole-4-carboxamide;
1-methyl-N-(2-(2-(trifluoromethyl)pyrimidin-4-yl)-1H-pyrrolo[3,2-c]pyridin-6-yl)-1H-pyrazole-4-carboxamide;
1-methyl-N-(2-(2-methylpyridin-4-yl)-1H-pyrrolo[3,2-c]pyridin-6-yl)-1H-indazole-4-carboxamide;
N-(2-(2-methylpyridin-4-yl)-1H-pyrrolo[3,2-c]pyridin-6-yl)-1H-indazole-4-carboxamide;
5-methyl-N-(2-(2-methylpyridin-4-yl)-1H-pyrrolo[3,2-c]pyridin-6-yl)oxazole-4-carboxamide;
1,1-dimethyl-3-(2-(2-methylpyridin-4-yl)-1H-pyrrolo[3,2-c]pyridin-6-yl)urea;
N-(2-(2-methoxypyrimidin-4-yl)-1H-pyrrolo[3,2-c]pyridin-6-yl)-1,5-dimethyl-1H-pyrazole-3-carboxamide;
N-(2-(2-ethoxypyrimidin-4-yl)-1H-pyrrolo[3,2-c]pyridin-6-yl)-1-methyl-1H-pyrazole-4-carboxamide;
1-methyl-N-(2-(2-methylpyridin-4-yl)-1H-pyrrolo[3,2-c]pyridin-6-yl)-5-(trifluoromethyl)-1H-pyrazole-3-carboxamide;
3-methoxy-4-(2-methoxyethoxy)-N-(2-(2-methylpyridin-4-yl)-1H-pyrrolo[3,2-c]pyridin-6-yl)benzamide;
3,4-dimethoxy-N-(2-(2-methylpyridin-4-yl)-1H-pyrrolo[3,2-c]pyridin-6-yl)benzamide;
N-(2-(2-methoxypyrimidin-4-yl)-1H-pyrrolo[3,2-c]pyridin-6-yl)pivalamide;
N-(2-(2-methylpyridin-4-yl)-1H-pyrrolo[3,2-c]pyridin-6-yl)bicyclo[1.1.1]pentane-1-carboxamide;
1-methyl-N-(2-(4-(trifluoromethyl)pyridin-2-yl)-1H-pyrrolo[3,2-c]pyridin-6-yl)-1H-pyrazole-4-carboxamide;
N-(2-(4-cyanopyridin-2-yl)-1H-pyrrolo[3,2-c]pyridin-6-yl)-1-methyl-1H-pyrazole-4-carboxamide;
N-(2-(2-methylpyridin-4-yl)-1H-pyrrolo[3,2-c]pyridin-6-yl)pivalamide;
N-(1-methyl-2-(2-(trifluoromethyl)pyrimidin-4-yl)-1H-pyrrolo[3,2-c]pyridin-6-yl)cyclopropanecarboxamide;
N-(2-(5-cyanopyrazin-2-yl)-1H-pyrrolo[3,2-c]pyridin-6-yl)-1-methyl-1H-pyrazole-4-carboxamide;
1-methyl-N-(2-(5-(trifluoromethyl)pyrazin-2-yl)-1H-pyrrolo[3,2-c]pyridin-6-yl)-1H-pyrazole-4-carboxamide;
1,5-dimethyl-N-(2-(2-(2,2,2-trifluoroethylamino)pyridin-4-yl)-1H-pyrrolo[3,2-c]pyridin-6-yl)-1H-pyrazole-3-carboxamide;
1-methyl-N-(2-(6-(trifluoromethyl)pyridin-3-yl)-1H-pyrrolo[3,2-c]pyridin-6-yl)-1H-pyrazole-4-carboxamide;
(S)-2-hydroxy-N-(2-(2-methylpyridin-4-yl)-1H-pyrrolo[3,2-c]pyridin-6-yl)propanamide;
1-methyl-N-(2-(2-(2,2,2-trifluoroethoxy)pyrimidin-4-yl)-1H-pyrrolo[3,2-c]pyridin-6-yl)-1H-pyrazole-4-carboxamide;
1-isopropyl-N-(2-(2-methoxypyrimidin-4-yl)-1-methyl-1H-pyrrolo[3,2-c]pyridin-6-yl)-1H-pyrazole-4-carboxamide;
N-(2-(2-(2,2-difluoroethoxy)pyrimidin-4-yl)-1H-pyrrolo[3,2-c]pyridin-6-yl)-1-methyl-1H-pyrazole-4-carboxamide;
N-(2-(2-isobutoxypyrimidin-4-yl)-1H-pyrrolo[3,2-c]pyridin-6-yl)-1-methyl-1H-pyrazole-4-carboxamide;
1-methyl-N-(2-(2-propoxypyrimidin-4-yl)-1H-pyrrolo[3,2-c]pyridin-6-yl)-1H-pyrazole-4-carboxamide;
N-(2-(2-(cyclobutylmethoxy)pyrimidin-4-yl)-1H-pyrrolo[3,2-c]pyridin-6-yl)-1-methyl-1H-pyrazole-4-carboxamide;
(R)-1-methyl-N-(2-(2-(1,1,1-trifluoropropan-2-yloxy)pyrimidin-4-yl)-1H-pyrrolo[3,2-c]pyridin-6-yl)-1H-pyrazole-4-carboxamide;
N-(2-(2-methoxy-6-methylpyrimidin-4-yl)-1H-pyrrolo[3,2-c]pyridin-6-yl)-1-methyl-1H-pyrazole-4-carboxamide;
N-(2-(2-ethoxypyrimidin-4-yl)-1-isobutyl-1H-pyrrolo[3,2-c]pyridin-6-yl)-1-methyl-1H-pyrazole-4-carboxamide;
N-(2-(2-(2,2-difluoroethoxy)pyrimidin-4-yl)-1H-pyrrolo[3,2-c]pyridin-6-yl)-1,3-dimethyl-1H-pyrazole-4-carboxamide;
N-(2-(2-(2,2-difluoroethoxy)pyrimidin-4-yl)-1H-pyrrolo[3,2-c]pyridin-6-yl)-1-ethyl-3-methyl-1H-pyrazole-4-carboxamide;
1-methyl-N-(2-(2-(neopentyloxy)pyrimidin-4-yl)-1H-pyrrolo[3,2-c]pyridin-6-yl)-1H-pyrazole-4-carboxamide;
N-(2-(2-methoxypyrimidin-4-yl)-1H-pyrrolo[3,2-c]pyridin-6-yl)-4-morpholinobenzamide;
N-(2-(2-ethoxypyrimidin-4-yl)-1H-pyrrolo[3,2-c]pyridin-6-yl)-3,4-dimethoxybenzamide;
(S)—N-(2-(2-ethoxypyrimidin-4-yl)-1H-pyrrolo[3,2-c]pyridin-6-yl)-3-methyl-1-((tetrahydrofuran-2-yl)methyl)-1H-pyrazole-4-carboxamide;
N-(2-(2-(2,2-difluoroethoxy)pyrimidin-4-yl)-1H-pyrrolo[3,2-c]pyridin-6-yl)-1-((1s,4s)-4-hydroxycyclohexyl)-1H-pyrazole-4-carboxamide;
N-(2-(2-(3-hydroxycyclobutoxy)pyrimidin-4-yl)-1H-pyrrolo[3,2-c]pyridin-6-yl)-1-methyl-1H-pyrazole-4-carboxamide;
N-(2-(2-((1s,4s)-4-hydroxycyclohexyloxy)pyrimidin-4-yl)-1H-pyrrolo[3,2-c]pyridin-6-yl)-1-methyl-1H-pyrazole-4-carboxamide;
(S)—N-(2-(2-methoxypyrimidin-4-yl)-1H-pyrrolo[3,2-c]pyridin-6-yl)-3-methyl-1-((tetrahydrofuran-3-yl)methyl)-1H-pyrazole-4-carboxamide;
N-(2-methyl-1H-pyrrolo[3,2-c]pyridin-6-yl)cyclopropanecarboxamide;
1-methyl-N-(2-(6-methylpyrimidin-4-yl)-1H-pyrrolo[3,2-c]pyridin-6-yl)-1H-pyrazole-4-carboxamide;
N-(1-isopropyl-2-(2-methylpyridin-4-yl)-1H-pyrrolo[3,2-c]pyridin-6-yl)cyclopropanecarboxamide;
N-(2-(1H-imidazol-5-yl)-1H-pyrrolo[3,2-c]pyridin-6-yl)cyclopropanecarboxamide;
1-methyl-N-(2-(1-methyl-1H-pyrazol-4-yl)-1H-pyrrolo[3,2-c]pyridin-6-yl)-1H-pyrazole-4-carboxamide;
1-methyl-N-(2-(pyridin-3-yl)-1H-pyrrolo[3,2-c]pyridin-6-yl)-1H-pyrazole-4-carboxamide;
N-(1-(2-(dimethylamino)ethyl)-2-(2-methylpyridin-4-yl)-1H-pyrrolo[3,2-c]pyridin-6-yl)cyclopropanecarboxamide;
N-(2-(5-fluoropyridin-2-yl)-1H-pyrrolo[3,2-c]pyridin-6-yl)-1-methyl-1H-pyrazole-4-carboxamide;
N-(2-(2-methyloxazol-4-yl)-1H-pyrrolo[3,2-c]pyridin-6-yl)cyclopropanecarboxamide;
1-methyl-N-(2-(1-methylpiperidin-4-yl)-1H-pyrrolo[3,2-c]pyridin-6-yl)-1H-pyrazole-4-carboxamide;
N-(3-methyl-5-(1H-1,2,4-triazol-1-yl)phenyl)-2-(pyridin-3-yl)-1H-pyrrolo[3,2-c]pyridin-6-amine;
N-(1-isopropyl-2-(2-(2,2,2-trifluoroethylamino)pyrimidin-4-yl)-1H-pyrrolo[3,2-c]pyridin-6-yl)-1-methyl-1H-pyrazole-4-carboxamide;
4-(6-(3-methyl-5-(1H-1,2,4-triazol-1-yl)phenylamino)-1H-pyrrolo[3,2-c]pyridin-2-yl)picolinonitrile;
N-(2-(4-fluoro-3-methylphenyl)-1H-pyrrolo[3,2-c]pyridin-6-yl)cyclopropanecarboxamide;
1-methyl-N-(2-(trifluoromethyl)-1H-pyrrolo[3,2-c]pyridin-6-yl)-1H-pyrazole-4-carboxamide;
N-(1-methyl-2-(1-methyl-1H-imidazol-4-yl)-1H-pyrrolo[3,2-c]pyridin-6-yl)cyclopropanecarboxamide;
N-(1-(2-hydroxyethyl)-2-(2-methoxypyrimidin-4-yl)-1H-pyrrolo[3,2-c]pyridin-6-yl)cyclopropanecarboxamide;
N-(1-ethyl-2-(2-methoxypyrimidin-4-yl)-1H-pyrrolo[3,2-c]pyridin-6-yl)cyclopropanecarboxamide;
N-(1-methyl-2-(1-methyl-1H-pyrazol-3-yl)-1H-pyrrolo[3,2-c]pyridin-6-yl)cyclopropanecarboxamide;
N-(1-methyl-2-m-tolyl-1H-pyrrolo[3,2-c]pyridin-6-yl)cyclopropanecarboxamide;
N-(1-methyl-2-(5-methylpyridin-2-yl)-1H-pyrrolo[3,2-c]pyridin-6-yl)cyclopropanecarboxamide;
1-methyl-2-(1-methyl-1H-imidazol-4-yl)-N-(tetrahydro-2H-pyran-4-yl)-1H-pyrrolo[3,2-c]pyridin-6-amine;
2-(pyridin-2-yl)-N-(tetrahydro-2H-pyran-4-yl)-1H-pyrrolo[3,2-c]pyridin-6-amine;
2-(4-methylpyridin-2-yl)-N-(tetrahydro-2H-pyran-4-yl)-1H-pyrrolo[3,2-c]pyridin-6-amine;
N-(tetrahydro-2H-pyran-4-yl)-2-p-tolyl-1H-pyrrolo[3,2-c]pyridin-6-amine;
N-(tetrahydro-2H-pyran-4-yl)-2-m-tolyl-1H-pyrrolo[3,2-c]pyridin-6-amine;
2-(pyridin-4-yl)-N-(tetrahydro-2H-pyran-4-yl)-1H-pyrrolo[3,2-c]pyridin-6-amine;
2-(1-methyl-1H-pyrazol-4-yl)-N-(tetrahydro-2H-pyran-4-yl)-1H-pyrrolo[3,2-c]pyridin-6-amine;
(S)-2-(2-methylpyridin-4-yl)-N-(tetrahydro-2H-pyran-3-yl)-1H-pyrrolo[3,2-c]pyridin-6-amine;
(R)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-o-tolyl-1H-pyrrolo[3,2-c]pyridin-6-amine;
1-methyl-N-(tetrahydro-2H-pyran-4-yl)-2-(2-(2,2,2-trifluoroethylamino)pyrimidin-4-yl)-1H-pyrrolo[3,2-c]pyridin-6-amine;
2-isopropyl-N-(tetrahydro-2H-pyran-4-yl)-1H-pyrrolo[3,2-c]pyridin-6-amine;
N-(tetrahydro-2H-pyran-4-yl)-2-(6-(2,2,2-trifluoroethylamino)pyrimidin-4-yl)-1H-pyrrolo[3,2-c]pyridin-6-amine;
2-ethyl-N-(tetrahydro-2H-pyran-4-yl)-1H-pyrrolo[3,2-c]pyridin-6-amine;
3-methyl-2-(2-methylpyridin-4-yl)-N-(tetrahydro-2H-pyran-4-yl)-1H-pyrrolo[3,2-c]pyridin-6-amine;
N-(tetrahydro-2H-pyran-4-yl)-2-(2-(2,2,2-trifluoroethylamino)pyridin-4-yl)-1H-pyrrolo[3,2-c]pyridin-6-amine;
2-cyclohexyl-N-(tetrahydro-2H-pyran-4-yl)-1H-pyrrolo[3,2-c]pyridin-6-amine;
1-methyl-N-(tetrahydro-2H-pyran-4-yl)-2-(6-(2,2,2-trifluoroethylamino)pyrimidin-4-yl)-1H-pyrrolo[3,2-c]pyridin-6-amine;
(R)—N-(tetrahydrofuran-3-yl)-2-(2-(2,2,2-trifluoroethylamino)pyrimidin-4-yl)-1H-pyrrolo[3,2-c]pyridin-6-amine;
(S)-3-methyl-2-(2-methylpyridin-4-yl)-N-(tetrahydrofuran-3-yl)-1H-pyrrolo[3,2-c]pyridin-6-amine;
(R)-3-methyl-2-(2-methylpyridin-4-yl)-N-(tetrahydrofuran-3-yl)-1H-pyrrolo[3,2-c]pyridin-6-amine;
N-cyclohexyl-3-methyl-2-(2-methylpyridin-4-yl)-1H-pyrrolo[3,2-c]pyridin-6-amine;
2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
1-methyl-2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2-(pyrrolidin-1-yl)pyrimidin-4-yl)-N-(tetrahydro-2H-pyran-4-yl)-1H-pyrrolo[3,2-c]pyridin-6-amine;
N-cyclobutyl-2-(2-(2,2,2-trifluoroethylamino)pyrimidin-4-yl)-1H-pyrrolo[3,2-c]pyridin-6-amine;
(S)—N-(tetrahydrofuran-3-yl)-2-(2-(2,2,2-trifluoroethylamino)pyrimidin-4-yl)-1H-pyrrolo[3,2-c]pyridin-6-amine;
2-(2-methylpyridin-4-yl)-N-(tetrahydro-2H-pyran-4-yl)-3-(trifluoromethyl)-1H-pyrrolo[3,2-c]pyridin-6-amine;
N-(tetrahydro-2H-pyran-4-yl)-3-(trifluoromethyl)-1H-pyrrolo[3,2-c]pyridin-6-amine;
2-(2-ethoxypyrimidin-4-yl)-N-(tetrahydro-2H-pyran-4-yl)-1H-pyrrolo[3,2-c]pyridin-6-amine;
1-methyl-2-(2-methylpyrimidin-4-yl)-N-(tetrahydro-2H-pyran-4-yl)-1H-pyrrolo[3,2-c]pyridin-6-amine;
(S)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(2,2,2-trifluoroethoxy)pyrimidin-4-yl)-1H-pyrrolo[3,2-c]pyridin-6-amine;
2-(2-(methyl(2,2,2-trifluoroethyl)amino)pyridin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(2-(2,2,2-trifluoroethylamino)pyridin-4-yl)-1H-pyrrolo[3,2-c]pyridin-6-amine;
N-isopropyl-1-methyl-2-(2-(2,2,2-trifluoroethylamino)pyrimidin-4-yl)-1H-pyrrolo[3,2-c]pyridin-6-amine;
N-tert-butyl-1-methyl-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(3,3,3-trifluoropropyl)pyrimidin-4-yl)-1H-pyrrolo[3,2-c]pyridin-6-amine;
(R)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
2-(2-ethylpyrimidin-4-yl)-1-methyl-N-(tetrahydro-2H-pyran-4-yl)-1H-pyrrolo[3,2-c]pyridin-6-amine;
2-(6-methoxypyrimidin-4-yl)-1-methyl-N-(tetrahydro-2H-pyran-4-yl)-1H-pyrrolo[3,2-c]pyridin-6-amine;
2-(6-ethylpyrimidin-4-yl)-1-methyl-N-(tetrahydro-2H-pyran-4-yl)-1H-pyrrolo[3,2-c]pyridin-6-amine;
N,1-dimethyl-2-(2-(2,2,2-trifluoroethylamino)pyrimidin-4-yl)-1H-pyrrolo[3,2-c]pyridin-6-amine;
2-(2-isopropylpyrimidin-4-yl)-1-methyl-N-(tetrahydro-2H-pyran-4-yl)-1H-pyrrolo[3,2-c]pyridin-6-amine;
1-methyl-N-(tetrahydro-2H-pyran-4-yl)-2-(6-(trifluoromethyl)pyrimidin-4-yl)-1H-pyrrolo[3,2-c]pyridin-6-amine;
2-(2,6-dimethylpyrimidin-4-yl)-N-(tetrahydro-2H-pyran-4-yl)-1H-pyrrolo[3,2-c]pyridin-6-amine;
2-(6-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(6-isopropylpyrimidin-4-yl)-1-methyl-N-(tetrahydro-2H-pyran-4-yl)-1H-pyrrolo[3,2-c]pyridin-6-amine;
1-methyl-N-(tetrahydro-2H-pyran-4-yl)-2-(2-(trifluoromethyl)pyrimidin-4-yl)-1H-pyrrolo[3,2-c]pyridin-6-amine;
and pharmaceutically acceptable salts, tautomers, isotopologues, or stereoisomers thereof.

15. A compound chosen from:
2-(2-methylpyridin-4-yl)-N-(tetrahydro-2H-pyran-4-yl)-1H-pyrrolo[3,2-c]pyridin-6-amine;
2-(6-methylpyrimidin-4-yl)-N-(tetrahydro-2H-pyran-4-yl)-1H-pyrrolo[3,2-c]pyridin-6-amine;
1-methyl-2-(6-methylpyrimidin-4-yl)-N-(tetrahydro-2H-pyran-4-yl)-1H-pyrrolo[3,2-c]pyridin-6-amine;
(R)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-(2-(2,2,2-trifluoroethylamino)pyridin-4-yl)-1H-pyrrolo[3,2-c]pyridin-6-amine;
N-(2-(2-methylpyridin-4-yl)-1H-pyrrolo[3,2-c]pyridin-6-yl)cyclopropanecarboxamide;
(R)-1-methyl-N-(2-(2-(1,1,1-trifluoropropan-2-ylamino)pyrimidin-4-yl)-1H-pyrrolo[3,2-c]pyridin-6-yl)-1H-pyrazole-4-carboxamide;
N-(cyclopropylmethyl)-2-(2-methylpyridin-4-yl)-1H-pyrrolo[3,2-c]pyridin-6-amine;
4-(6-(1-methyl-1H-pyrazol-4-ylamino)-1H-pyrrolo[3,2-c]pyridin-2-yl)picolinonitrile
N-(tetrahydro-2H-pyran-4-yl)-2-(2-(2,2,2-trifluoroethylamino)pyrimidin-4-yl)-1H-pyrrolo[3,2-c]pyridin-6-amine;
4-(6-(3-(tetrahydro-2H-pyran-4-yl)phenylamino)-1H-pyrrolo[3,2-c]pyridin-2-yl)picolinonitrile;
N-(3-methyl-5-(1H-1,2,4-triazol-1-yl)phenyl)-2-(2-(2,2,2-trifluoroethylamino)pyrimidin-4-yl)-1H-pyrrolo[3,2-c]pyridin-6-amine;
(R)-2-(2-methylpyridin-4-yl)-N-(tetrahydrofuran-3-yl)-1H-pyrrolo[3,2-c]pyridin-6-amine;
2-(2-methoxypyrimidin-4-yl)-N-(tetrahydro-2H-pyran-4-yl)-1H-pyrrolo[3,2-c]pyridin-6-amine;
1-methyl-2-(2-methylpyridin-4-yl)-N-(tetrahydro-2H-pyran-4-yl)-1H-pyrrolo[3,2-c]pyridin-6-amine;
2-(2-methylpyrimidin-4-yl)-N-(tetrahydro-2H-pyran-4-yl)-1H-pyrrolo[3,2-c]pyridin-6-amine;
2-(2-methylpyridin-4-yl)-N-(4-methyltetrahydro-2H-pyran-4-yl)-1H-pyrrolo[3,2-c]pyridin-6-amine;
(S)-2-(2-methylpyridin-4-yl)-N-(tetrahydrofuran-3-yl)-1H-pyrrolo[3,2-c]pyridin-6-amine;
(S)-2-(2-methylpyridin-4-yl)-N-(tetrahydrofuran-3-yl)-1H-pyrrolo[3,2-c]pyridin-6-amine;
2-(pyridin-4-yl)-N-(tetrahydro-2H-pyran-4-yl)-1H-pyrrolo[3,2-c]pyridin-6-amine;
(S)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-o-tolyl-1H-pyrrolo[3,2-c]pyridin-6-amine;
1-methyl-N-(tetrahydro-2H-pyran-4-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(6-(2,2,2-trifluoroethylamino)pyrimidin-4-yl)-1H-pyrrolo[3,2-c]pyridin-6-amine;
2-cyclohexyl-N-(tetrahydro-2H-pyran-4-yl)-1H-pyrrolo[3,2-c]pyridin-6-amine;
1-methyl-N-(tetrahydro-2H-pyran-4-yl)-2-(6-(2,2,2-trifluoroethylamino)pyrimidin-4-yl)-1H-pyrrolo[3,2-c]pyridin-6-amine;
(R)—N-(tetrahydrofuran-3-yl)-2-(2-(2,2,2-trifluoroethylamino)pyrimidin-4-yl)-1H-pyrrolo[3,2-c]pyridin-6-amine;
2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
1-methyl-2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2-(pyrrolidin-1-yl)pyrimidin-4-yl)-N-(tetrahydro-2H-pyran-4-yl)-1H-pyrrolo[3,2-c]pyridin-6-amine;
N-cyclobutyl-2-(2-(2,2,2-trifluoroethylamino)pyrimidin-4-yl)-1H-pyrrolo[3,2-c]pyridin-6-amine;
(S)—N-(tetrahydrofuran-3-yl)-2-(2-(2,2,2-trifluoroethylamino)pyrimidin-4-yl)-1H-pyrrolo[3,2-c]pyridin-6-amine;
2-(2-ethoxypyrimidin-4-yl)-N-(tetrahydro-2H-pyran-4-yl)-1H-pyrrolo[3,2-c]pyridin-6-amine;
1-methyl-2-(2-methylpyrimidin-4-yl)-N-(tetrahydro-2H-pyran-4-yl)-1H-pyrrolo[3,2-c]pyridin-6-amine;
(S)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(2,2,2-trifluoroethoxy)pyrimidin-4-yl)-1H-pyrrolo[3,2-c]pyridin-6-amine;
2-(2-(methyl(2,2,2-trifluoroethyl)amino)pyridin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(2-(2,2,2-trifluoroethylamino)pyridin-4-yl)-1H-pyrrolo[3,2-c]pyridin-6-amine;
N-isopropyl-1-methyl-2-(2-(2,2,2-trifluoroethylamino)pyrimidin-4-yl)-1H-pyrrolo[3,2-c]pyridin-6-amine;
N-tert-butyl-1-methyl-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(3,3,3-trifluoropropyl)pyrimidin-4-yl)-1H-pyrrolo[3,2-c]pyridin-6-amine;
(R)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
2-(6-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2,6-dimethylpyrimidin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(6-(trifluoromethyl)pyrimidin-4-yl)-1H-pyrrolo[3,2-c]pyridin-6-amine;
2-(2-isopropylpyrimidin-4-yl)-1-methyl-N-(tetrahydro-2H-pyran-4-yl)-1H-pyrrolo[3,2-c]pyridin-6-amine;
N,1-dimethyl-2-(2-(2,2,2-trifluoroethylamino)pyrimidin-4-yl)-1H-pyrrolo[3,2-c]pyridin-6-amine;
2-(6-ethylpyrimidin-4-yl)-1-methyl-N-(tetrahydro-2H-pyran-4-yl)-1H-pyrrolo[3,2-c]pyridin-6-amine;
2-(6-methoxypyrimidin-4-yl)-1-methyl-N-(tetrahydro-2H-pyran-4-yl)-1H-pyrrolo[3,2-c]pyridin-6-amine;
2-(2-ethylpyrimidin-4-yl)-1-methyl-N-(tetrahydro-2H-pyran-4-yl)-1H-pyrrolo[3,2-c]pyridin-6-amine;
and pharmaceutically acceptable salts, tautomers, isotopologues, or stereoisomers thereof.

16. A compound chosen from:
2-(2-methylpyridin-4-yl)-N-(tetrahydro-2H-pyran-4-yl)-1H-pyrrolo[3,2-c]pyridin-6-amine;
2-(6-methylpyrimidin-4-yl)-N-(tetrahydro-2H-pyran-4-yl)-1H-pyrrolo[3,2-c]pyridin-6-amine;
1-methyl-2-(6-methylpyrimidin-4-yl)-N-(tetrahydro-2H-pyran-4-yl)-1H-pyrrolo[3,2-c]pyridin-6-amine;
(R)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-(2-(2,2,2-trifluoroethylamino)pyridin-4-yl)-1H-pyrrolo[3,2-c]pyridin-6-amine;
N-(2-(2-methylpyridin-4-yl)-1H-pyrrolo[3,2-c]pyridin-6-yl)cyclopropanecarboxamide;
(R)-1-methyl-N-(2-(2-(1,1,1-trifluoropropan-2-ylamino)pyrimidin-4-yl)-1H-pyrrolo[3,2-c]pyridin-6-yl)-1H-pyrazole-4-carboxamide;
and pharmaceutically acceptable salts, tautomers, isotopologues, or stereoisomers thereof.

17. A compound chosen from:
N-(cyclopropylmethyl)-2-(2-methylpyridin-4-yl)-1H-pyrrolo[3,2-c]pyridin-6-amine;
4-(6-(1-methyl-1H-pyrazol-4-ylamino)-1H-pyrrolo[3,2-c]pyridin-2-yl)picolinonitrile;
N-(tetrahydro-2H-pyran-4-yl)-2-(2-(2,2,2-trifluoroethylamino)pyrimidin-4-yl)-1H-pyrrolo[3,2-c]pyridin-6-amine;
4-(6-(3-(tetrahydro-2H-pyran-4-yl)phenylamino)-1H-pyrrolo[3,2-c]pyridin-2-yl)picolinonitrile;
N-(3-methyl-5-(1H-1,2,4-triazol-1-yl)phenyl)-2-(2-(2,2,2-trifluoroethylamino)pyrimidin-4-yl)-1H-pyrrolo[3,2-c]pyridin-6-amine;
(R)-2-(2-methylpyridin-4-yl)-N-(tetrahydrofuran-3-yl)-1H-pyrrolo[3,2-c]pyridin-6-amine;
2-(2-methoxypyrimidin-4-yl)-N-(tetrahydro-2H-pyran-4-yl)-1H-pyrrolo[3,2-c]pyridin-6-amine;
1-methyl-2-(2-methylpyridin-4-yl)-N-(tetrahydro-2H-pyran-4-yl)-1H-pyrrolo[3,2-c]pyridin-6-amine;
2-(2-methylpyrimidin-4-yl)-N-(tetrahydro-2H-pyran-4-yl)-1H-pyrrolo[3,2-c]pyridin-6-amine;
2-(2-methylpyridin-4-yl)-N-(4-methyltetrahydro-2H-pyran-4-yl)-1H-pyrrolo[3,2-c]pyridin-6-amine;
(S)-2-(2-methylpyridin-4-yl)-N-(tetrahydrofuran-3-yl)-1H-pyrrolo[3,2-c]pyridin-6-amine;
(S)-2-(2-methylpyridin-4-yl)-N-(tetrahydrofuran-3-yl)-1H-pyrrolo[3,2-c]pyridin-6-amine;
2-(pyridin-4-yl)-N-(tetrahydro-2H-pyran-4-yl)-1H-pyrrolo[3,2-c]pyridin-6-amine;
(S)-2-(2-methylpyridin-4-yl)-N-(tetrahydro-2H-pyran-3-yl)-1H-pyrrolo[3,2-c]pyridin-6-amine;
N-(tetrahydro-2H-pyran-4-yl)-2-o-tolyl-1H-pyrrolo[3,2-c]pyridin-6-amine;
1-methyl-N-(tetrahydro-2H-pyran-4-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(6-(2,2,2-trifluoroethylamino)pyrimidin-4-yl)-1H-pyrrolo[3,2-c]pyridin-6-amine;
2-cyclohexyl-N-(tetrahydro-2H-pyran-4-yl)-1H-pyrrolo[3,2-c]pyridin-6-amine;
1-methyl-N-(tetrahydro-2H-pyran-4-yl)-2-(6-(2,2,2-trifluoroethylamino)pyrimidin-4-yl)-1H-pyrrolo[3,2-c]pyridin-6-amine;
(R)—N-(tetrahydrofuran-3-yl)-2-(2-(2,2,2-trifluoroethylamino)pyrimidin-4-yl)-1H-pyrrolo[3,2-c]pyridin-6-amine;
2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
1-methyl-2-(2-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2-(pyrrolidin-1-yl)pyrimidin-4-yl)-N-(tetrahydro-2H-pyran-4-yl)-1H-pyrrolo[3,2-c]pyridin-6-amine;
N-cyclobutyl-2-(2-(2,2,2-trifluoroethylamino)pyrimidin-4-yl)-1H-pyrrolo[3,2-c]pyridin-6-amine;
(S)—N-(tetrahydrofuran-3-yl)-2-(2-(2,2,2-trifluoroethylamino)pyrimidin-4-yl)-1H-pyrrolo[3,2-c]pyridin-6-amine;
2-(2-ethoxypyrimidin-4-yl)-N-(tetrahydro-2H-pyran-4-yl)-1H-pyrrolo[3,2-c]pyridin-6-amine;
1-methyl-2-(2-methylpyrimidin-4-yl)-N-(tetrahydro-2H-pyran-4-yl)-1H-pyrrolo[3,2-c]pyridin-6-amine;
(S)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(2,2,2-trifluoroethoxy)pyrimidin-4-yl)-1H-pyrrolo[3,2-c]pyridin-6-amine;
2-(2-(methyl(2,2,2-trifluoroethyl)amino)pyridin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(2-(2,2,2-trifluoroethylamino)pyridin-4-yl)-1H-pyrrolo[3,2-c]pyridin-6-amine;
N-isopropyl-1-methyl-2-(2-(2,2,2-trifluoroethylamino)pyrimidin-4-yl)-1H-pyrrolo[3,2-c]pyridin-6-amine;
N-tert-butyl-1-methyl-2-(2-(2,2,2-trifluoroethylamino)pyrimidin-4-yl)-1H-pyrrolo[3,2-c]pyridin-6-amine;
N-(tetrahydro-2H-pyran-4-yl)-2-(2-(3,3,3-trifluoropropyl)pyrimidin-4-yl)-1H-pyrrolo[3,2-c]pyridin-6-amine;
(R)-1-methyl-N-(tetrahydro-2H-pyran-3-yl)-2-(2-(2,2,2-trifluoroethylamino)pyrimidin-4-yl)-1H-pyrrolo[3,2-c]pyridin-6-amine;
2-(6-(methyl(2,2,2-trifluoroethyl)amino)pyrimidin-4-yl)-N-(tetrahydro-2H-pyran-4-yl)-1H-pyrrolo[3,2-c]pyridin-6-amine;
2-(2,6-dimethylpyrimidin-4-yl)-N-(tetrahydro-2H-pyran-4-yl)-1H-pyrrolo[3,2-c]pyridin-6-amine;
1-methyl-N-(tetrahydro-2H-pyran-4-yl)-2-(6-(trifluoromethyl)pyrimidin-4-yl)-1H-pyrrolo[3,2-c]pyridin-6-amine;
2-(2-isopropylpyrimidin-4-yl)-1-methyl-N-(tetrahydro-2H-pyran-4-yl)-1H-pyrrolo[3,2-c]pyridin-6-amine;
N,1-dimethyl-2-(2-(2,2,2-trifluoroethylamino)pyrimidin-4-yl)-1H-pyrrolo[3,2-c]pyridin-6-amine;
2-(6-ethylpyrimidin-4-yl)-1-methyl-N-(tetrahydro-2H-pyran-4-yl)-1H-pyrrolo[3,2-c]pyridin-6-amine;
2-(6-methoxypyrimidin-4-yl)-1-methyl-N-(tetrahydro-2H-pyran-4-yl)-1H-pyrrolo[3,2-c]pyridin-6-amine;
2-(2-ethylpyrimidin-4-yl)-1-methyl-N-(tetrahydro-2H-pyran-4-yl)-1H-pyrrolo[3,2-c]pyridin-6-amine;
and pharmaceutically acceptable salts, tautomers, isotopologues, or stereoisomers thereof.

18. The compound according to claim 16, wherein the compound is N-(tetrahydro-2H-pyran-4-yl)-2-(2-(2,2,2-trifluoroethylamino)pyridin-4-yl)-1H-pyrrolo[3,2-c]pyridin-6-amine.

19. The compound according to claim 16, wherein the compound is (R)-2-(2-methylpyridin-4-yl)-N-(tetrahydro-2H-pyran-3-yl)-1H-pyrrolo[3,2-c]pyridin-6-amine.

20. The compound according to claim 16, wherein the compound is 2-(6-methylpyrimidin-4-yl)-N-(tetrahydro-2H-pyran-4-yl)-1H-pyrrolo[3,2-c]pyridin-6-amine.

21. The compound according to claim 16, wherein the compound is 2-(2-methylpyridin-4-yl)-N-(tetrahydro-2H-pyran-4-yl)-1H-pyrrolo[3,2-c]pyridin-6-amine.

22. A pharmaceutical composition comprising an effective amount of a compound of any of claims 1 to 17 or pharmaceutically acceptable salt, tautomer, isotopologue, or stereoisomer thereof, and at least one pharmaceutically acceptable carrier, excipient or vehicle.

23. A method of treating cryptosporidiosis, comprising administering to a subject in need thereof an effective amount of a compound of any of claims 1 to 21 or pharmaceutically acceptable salt, tautomer, isotopologue, or stereoisomer thereof.

24. A method of inhibiting a parasite or parasitic activity in a subject, comprising administering to a subject in need thereof an effective amount of a compound of any of claims 1 to 17 or pharmaceutically acceptable salt, tautomer, isotopologue, or stereoisomer thereof.

25. The method of claim 24, wherein the parasite is Cryptosporidium parvum.

26. The method of claim 24, wherein the parasite is Cryptosporidium hominis.

27. The use of a compound of any of claims 1 to 21 in the manufacture if a medicament for the treatment of cryptosporidiosis.

28. The use of a compound of any of claims 1 to 21 in the manufacture if a medicament for the inhibition of a parasite or parasitic activity.

29. The use according to claim 28, wherein the parasite is Cryptosporidium parvum.

30. The use according to claim 28, wherein the parasite is Cryptosporidium hominis.
